(19) United States
(12) Patent Application Publication
(10) Pub. No.: US 2026/0007787 A1
(43) Pub. Date: Jan. 8, 2026

(54) RADIOPHARMACEUTICAL SMALL MOLECULE INHIBITORS OF FIBROBLAST ACTIVATION PROTEIN FOR TARGETED THERAPY AND DIAGNOSTIC IMAGING OF DESMOPLASTIC TUMORS

(71) Applicant: Trustees of Tufts College, Medford, MA (US)

(72) Inventors: William W. Bachovchin, Cambridge, MA (US); Hung-sen Lai, Andover, MA (US); Wengen Wu, Winchester, MA (US)

(21) Appl. No.: 19/259,142

(22) Filed: Jul. 3, 2025

Related U.S. Application Data
(60) Provisional application No. 63/667,439, filed on Jul. 3, 2024.
(60) Provisional application No. 63/758,785, filed on Feb. 14, 2025.
(60) Provisional application No. 63/811,130, filed on May 23, 2025.

(51) Int. Cl. A61K 51/08; A61K 45/06

(52) U.S. Cl. CPC A61K 51/088; A61K 45/06

(57) ABSTRACT

Disclosed are compounds of the formula:

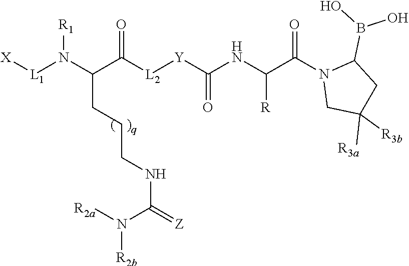

wherein X, L₁, R, R₁, Z, R₂ₐ, R_{2b}, L₂, Y, R₃ₐ, R_{3b}, and q are as described herein, pharmaceutically acceptable salts thereof, and methods of using these compounds and pharmaceutically acceptable salts thereof for treating patients for cancer.

### RELATED APPLICATIONS

This application claims the benefit of priority to U.S. Provisional Application No. 63/811,130, filed May 23, 2025; U.S. Provisional Application No. 63/758,785, filed Feb. 14, 2025; and U.S. Provisional Application No. 63/667,439, filed Jul. 3, 2024.

### BACKGROUND OF THE INVENTION

Fibroblast Activation Protein (FAP) is a type II transmembrane serine protease from the dipeptidyl peptidase 4 family, known for its dual enzymatic roles in tissue remodeling and disease progression. While FAP is typically absent in healthy adult tissues, its expression significantly increases in the stroma of over 90% of epithelial carcinomas, particularly within cancer-associated fibroblasts (CAFs) in the tumor microenvironment. This pattern of expression, linked to poor patient outcomes, underscores the therapeutic potential of FAP, with Fibroblast Activation Protein Inhibitors (FAPi) emerging as a crucial development in oncological therapies.

The tumor microenvironment, especially the stromal component which can constitute more than 90% of the tumor mass, provides a foundational role for cancer proliferation and metastasis. CAFs enhance tumor growth, cellular migration, and disease progression through their interactions with cancer cells and the extracellular matrix. The use of FAP targeting in diagnostic imaging and endoradiotherapeutic strategies aims to disrupt this supportive environment and inhibit tumor growth.

Advancements have led to the development of FAP inhibitors designed for high specificity. These agents facilitate the precise delivery of diagnostic and therapeutic radionuclides directly to the tumor site, improving imaging accuracy and the effectiveness of radioligand therapies (RLTs) while aiming to minimize the impact on healthy tissues. Despite the potential of FAP inhibitor based RLTs, challenges remain. Ongoing research efforts are focused on addressing these challenges, including the development of albumin binder-conjugated FAP inhibitors and multivalent FAP inhibitor derivatives to improve systemic circulation and tumor uptake. Exploring radionuclides with shorter half-lives is being considered to optimize therapeutic efficacy while reducing toxicity.

Recent advances in FAP-targeted radiopharmaceuticals have demonstrated significant clinical progress, particularly with the introduction of 18F-labeled FAP inhibitors that can potentially offer superior imaging capabilities compared to traditional tracers in various cancers. However, transitioning from diagnostic applications to effective RLTs requires further refinement in FAP inhibitor design. This involves addressing resistance mechanisms, optimizing inhibitors to support an integrated immune response without eliciting adverse reactions, and improving the pharmacokinetic profile for sustained tumor targeting and minimized exposure of healthy tissues.

The prevalent role of FAP in cancer-associated fibroblasts across many epithelial tumors, along with its association with poor prognoses, emphasizes the importance of targeting FAP. Such targeting not only enhances the specificity and efficacy of therapies, reducing the impact on healthy cells but also supports the accurate diagnosis of malignancies. Accordingly, there remains a vital need for therapeutic agents that can effectively disrupt FAP's role in tumor support and progression.

### SUMMARY OF THE INVENTION

The novel compounds described herein utilize a unique targeting mechanism to enhance both diagnostic and therapeutic capabilities. These agents are specifically designed to bind to FAP expressed in tumor-associated stromal cells, facilitating targeted delivery and action of radiopharmaceuticals, thereby improving the specificity and effectiveness of cancer treatment protocols.

The present invention introduces an innovative approach to cancer diagnosis and therapy through the development of novel radiopharmaceuticals and imaging agents specifically designed to target Fibroblast Activation Protein (FAP). FAP, a marker highly expressed in cancer-associated fibroblasts (CAFs) found within the tumor stroma of over 90% of epithelial carcinomas, including but not limited to pancreatic, colon, and breast cancers, is virtually absent in healthy adult tissues. The significant presence of CAFs and their expression of FAP in the tumor microenvironment, coupled with the correlation between FAP expression and poor patient prognosis, underscores the potential of FAP as a strategic target for therapeutic intervention.

The present invention provides small-molecule radiopharmaceutical and imaging agents based on a FAP-specific inhibitor.

Compounds of Formula I are provided herein:

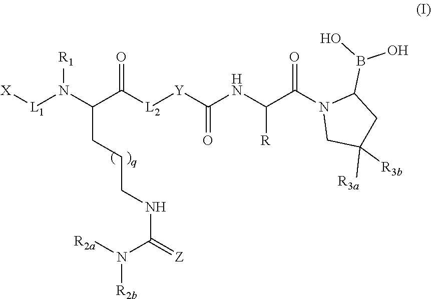

wherein:
X is a chelator suitable for radiolabeling, or a chelator bound to a radioactive atom or complex;
L₁ is a bond or a group of the formula:

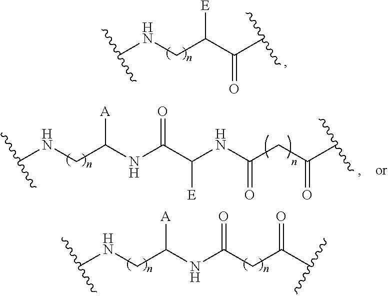

wherein each n is independently 0, 1, 2, 3, 4, or 5;
q is 0, 1, or 2;
Z is ═O or ═NR_{2c};
A is —H or —COOH;
E is —H,

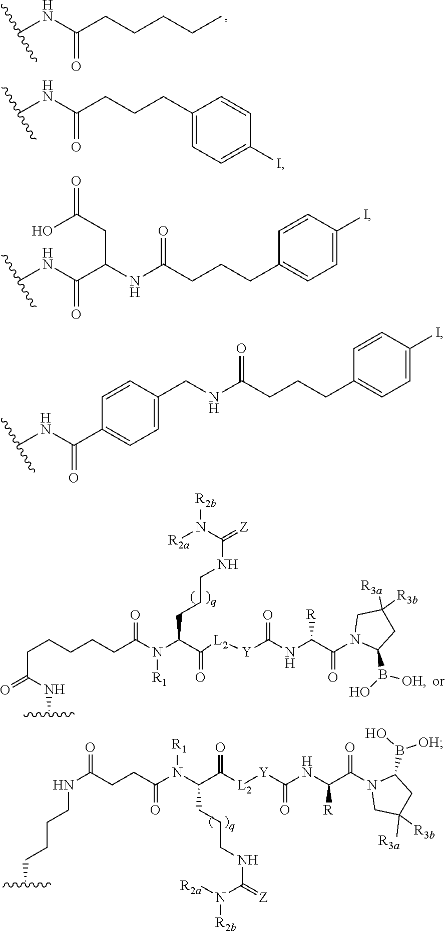

wherein each R is independently —H, methyl or —CH₂OH;
R₁ is —H or —C₁₋₃ alkyl;
R₂ₐ, R_{2b} and R_{2c} are each independently —H or —C₁₋₃ alkyl;
R₃ₐ and R_{3b} are each independently —H, —OH, or halogen; and
L₂ is a group of the formula:

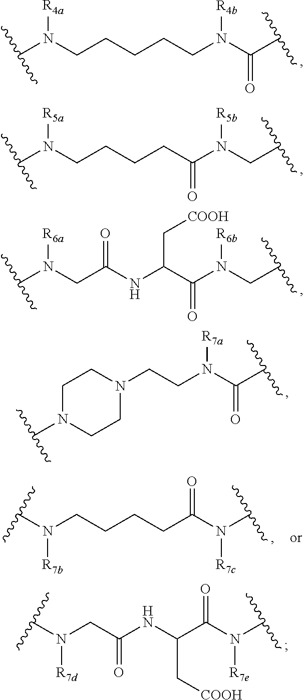

wherein R₄ₐ and R_{4b} are each independently —H or —C₁₋₃ alkyl;
R₅ₐ and R_{5b} are each independently —H or —C₁₋₃ alkyl
R₆ₐ and R_{6b} are each independently —H or —C₁₋₃ alkyl;
R₇ₐ is —H or —C₁₋₃ alkyl;
R_{7b} and R_{7c} are each independently —H or —C₁₋₃ alkyl; and
R_{7d} and R₇ₑ are each independently —H or —C₁₋₃ alkyl;
Y is selected from the group consisting of:

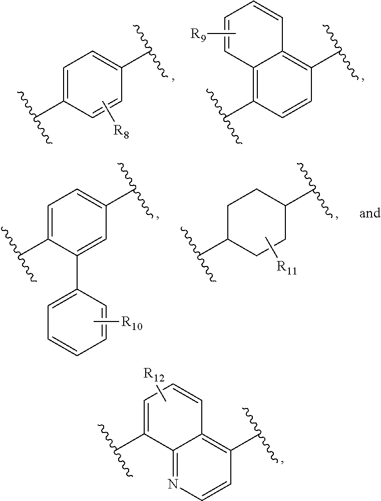

wherein R₈ is —H, —OH, halogen, —OR₈ₐ, —NR_{8b}R_{8c}, —COOH, or a 5 to 6 membered heteroaryl;
R₉ is —H, —OH, halogen, —OR₉ₐ, —NR_{9b}R_{9c}, or —COOH;
R₁₀ is —H, —OH, halogen, —OR₁₀ₐ, —NR_{10b}R_{10c}, or —COOH; and
R₁₁ is —H, —OH, halogen, —OR₁₁ₐ, —NR_{11b}R_{11c}, or —COOH;
wherein R₈ₐ, R_{8b}, and R_{8c} are each independently —H or —C₁₋₃ alkyl;
R₉ₐ, R_{9b}, and R_{9c} are each independently —H or —C₁₋₃ alkyl;
R₁₀ₐ, R_{10b}, and R_{10c} are each independently —H or —C₁₋₃ alkyl;
R₁₁ₐ, R_{11b}, and R_{11c} are each independently —H or —C₁₋₃ alkyl; and
R₁₂ is —H, —OH, halogen, —OR₉ₐ, —NR_{9b}R_{9c}, or —COOH;
or a pharmaceutically acceptable salt thereof.

Also provided herein are methods of using the compounds of Formula I, pharmaceutically acceptable salts thereof, and pharmaceutical compositions thereof, to treat cancer, in particular for the treatment of breast cancer, colorectal cancer, pancreatic cancer, non-small cell lung cancer, small cell lung cancer, prostate cancer, ovarian cancer, melanoma, liver cancer, gastric cancer, esophageal cancer, head and neck cancer, bladder cancer, renal cell carcinoma, glioblastoma, thyroid carcinoma, sarcoma, endometrial cancer, cervical cancer, testicular cancer, mesothelioma, neuroendocrine cancer, bone cancer, adenocarcinoma, non-melanoma skin cancer, leukemia, lymphoma, multiple myeloma, adrenal cancer, cholangiocarcinoma, squamous cell carcinoma, diffuse large B cell lymphoma, squamous cell carcinoma, basal cell carcinoma, transitional cell carcinoma, salivary gland tumors, thymoma, pituitary adenoma, Merkel cell carcinoma, peritoneal carcinoma, nasopharyngeal carcinoma, acute lymphoblastic leukemia, acute myeloid leukemia, chronic lymphocytic leukemia, chronic myeloid leukemia, Hodgkin's lymphoma, non-Hodgkin's lymphoma, uveal melanoma, cutaneous melanoma, mucosal melanoma, gastrointestinal stromal cancer, vulvar cancer, and/or fallopian tube carcinoma.

Further provided herein, are methods of using the compounds of Formula I, pharmaceutically acceptable salts thereof, and pharmaceutical compositions thereof, to treat cancer, in particular for the treatment of lung cancer, pancreatic cancer, cervical cancer, esophageal cancer, endometrial cancer, ovarian cancer, cholangiocarcinoma, and/or colorectal cancer.

Further provided herein, are methods of using the compounds of Formula I, pharmaceutically acceptable salts thereof, and pharmaceutical compositions thereof, to treat cancer, in particular for the treatment of breast cancer, colorectal cancer, pancreatic cancer, non-small cell lung cancer, small cell lung cancer, ovarian cancer, liver cancer, gastric cancer, esophageal cancer, head and neck cancer, bladder cancer, thyroid carcinoma, sarcoma, cervical cancer, bone cancer, cholangiocarcinoma, salivary gland tumors, gastrointestinal stromal cancer, vulvar cancer, and/or fallopian tube carcinoma.

Further provided herein, are methods of using the compounds of Formula I, pharmaceutically acceptable salts thereof, and pharmaceutical compositions thereof, to treat cancer, in particular for the treatment of breast cancer, lung cancer, pancreatic cancer, cervical cancer, esophageal cancer, endometrial cancer, ovarian cancer, cholangiocarcinoma, and/or colorectal cancer.

The methods include administering a therapeutically effective amount of a compound of Formula I, or a pharmaceutically acceptable salt thereof, and pharmaceutical compositions thereof, to a patient in need thereof.

Further provided herein, are compounds of Formula I, and pharmaceutically acceptable salts thereof, and for use in therapy. Additionally provided herein, are the compounds of Formula I, and pharmaceutically acceptable salts thereof, and pharmaceutical compositions thereof, for use in the treatment of cancer, in particular for the treatment of breast cancer, colorectal cancer, pancreatic cancer, non-small cell lung cancer, small cell lung cancer, prostate cancer, ovarian cancer, melanoma, liver cancer, gastric cancer, esophageal cancer, head and neck cancer, bladder cancer, renal cell carcinoma, glioblastoma, thyroid carcinoma, sarcoma, endometrial cancer, cervical cancer, testicular cancer, mesothelioma, neuroendocrine cancer, bone cancer, adenocarcinoma, non-melanoma skin cancer, leukemia, lymphoma, multiple myeloma, adrenal cancer, cholangiocarcinoma, squamous cell carcinoma, diffuse large B cell lymphoma, squamous cell carcinoma, basal cell carcinoma, transitional cell carcinoma, salivary gland tumors, thymoma, pituitary adenoma, Merkel cell carcinoma, peritoneal carcinoma, nasopharyngeal carcinoma, acute lymphoblastic leukemia, acute myeloid leukemia, chronic lymphocytic leukemia, chronic myeloid leukemia, Hodgkin's lymphoma, non-Hodgkin's lymphoma, uveal melanoma, cutaneous melanoma, mucosal melanoma, gastrointestinal stromal cancer, vulvar cancer, and/or fallopian tube carcinoma. Additionally provided herein, are the compounds of Formula I, and pharmaceutically acceptable salts thereof, and pharmaceutical compositions thereof, for use in the treatment of cancer, in particular for the treatment of lung cancer, pancreatic cancer, cervical cancer, esophageal cancer, endometrial cancer, ovarian cancer, cholangiocarcinoma, and/or colorectal cancer. Additionally provided herein, are the compounds of Formula I, and pharmaceutically acceptable salts thereof, and pharmaceutical compositions thereof, for use in the treatment of cancer, in particular for the treatment of breast cancer, colorectal cancer, pancreatic cancer, non-small cell lung cancer, small cell lung cancer, ovarian cancer, liver cancer, gastric cancer, esophageal cancer, head and neck cancer, bladder cancer, thyroid carcinoma, sarcoma, cervical cancer, bone cancer, cholangiocarcinoma, salivary gland tumors, gastrointestinal stromal cancer, vulvar cancer, or fallopian tube carcinoma. Additionally provided herein, are the compounds of Formula I, and pharmaceutically acceptable salts thereof, and pharmaceutical compositions thereof, for use in the treatment of cancer, in particular for the treatment of breast cancer, lung cancer, pancreatic cancer, cervical cancer, esophageal cancer, endometrial cancer, ovarian cancer, cholangiocarcinoma, and/or colorectal cancer.

Also additionally provided herein is the use of compounds of Formula I, or pharmaceutically acceptable salts thereof, and pharmaceutical compositions thereof, in the manufacture of a medicament for treating cancer, in particular for the treatment of breast cancer, colorectal cancer, pancreatic cancer, non-small cell lung cancer, small cell lung cancer, prostate cancer, ovarian cancer, melanoma, liver cancer, gastric cancer, esophageal cancer, head and neck cancer, bladder cancer, renal cell carcinoma, glioblastoma, thyroid carcinoma, sarcoma, endometrial cancer, cervical cancer, testicular cancer, mesothelioma, neuroendocrine cancer, bone cancer, adenocarcinoma, non-melanoma skin cancer, leukemia, lymphoma, multiple myeloma, adrenal cancer, cholangiocarcinoma, squamous cell carcinoma, diffuse large B cell lymphoma, squamous cell carcinoma, basal cell carcinoma, transitional cell carcinoma, salivary gland tumors, thymoma, pituitary adenoma, Merkel cell carcinoma, peritoneal carcinoma, nasopharyngeal carcinoma, acute lymphoblastic leukemia, acute myeloid leukemia, chronic lymphocytic leukemia, chronic myeloid leukemia, Hodgkin's lymphoma, non-Hodgkin's lymphoma, uveal melanoma, cutaneous melanoma, mucosal melanoma, gastrointestinal stromal cancer, vulvar cancer, and/or fallopian tube carcinoma.

Also additionally provided herein is the use of compounds of Formula I, or pharmaceutically acceptable salts thereof, and pharmaceutical compositions thereof, in the manufacture of a medicament for treating cancer, in particular for the treatment of lung cancer, pancreatic cancer, cervical cancer, esophageal cancer, endometrial cancer, ovarian cancer, cholangiocarcinoma, and/or colorectal cancer.

Also additionally provided herein is the use of compounds of Formula I, or pharmaceutically acceptable salts thereof, and pharmaceutical compositions thereof, in the manufacture of a medicament for treating cancer, in particular for the treatment of breast cancer, colorectal cancer, pancreatic cancer, non-small cell lung cancer, small cell lung cancer, ovarian cancer, liver cancer, gastric cancer, esophageal cancer, head and neck cancer, bladder cancer, thyroid carcinoma, sarcoma, cervical cancer, bone cancer, cholangiocarcinoma, salivary gland tumors, gastrointestinal stromal cancer, vulvar cancer, and/or fallopian tube carcinoma.

Also additionally provided herein is the use of compounds of Formula I, or pharmaceutically acceptable salts thereof, and pharmaceutical compositions thereof, in the manufacture of a medicament for treating cancer, in particular for the treatment of breast cancer, lung cancer, pancreatic cancer, cervical cancer, esophageal cancer, endometrial cancer, ovarian cancer, cholangiocarcinoma, and/or colorectal cancer.

### DETAILED DESCRIPTION OF THE INVENTION

Novel inhibitors of Fibroblast Activation Protein (FAP) are described herein. These new compounds could address the needs noted above for inhibitors of Fibroblast Activation Protein (FAP) in the treatment of certain cancers.

The present invention provides a compound of Formula I:

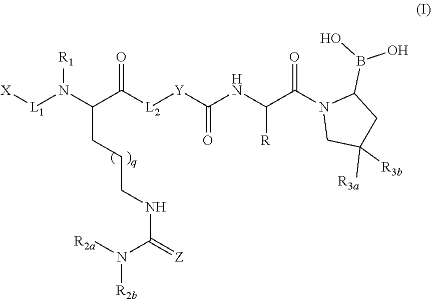

wherein X, L₁, R, R₁, R₂ₐ, R_{2b}, Z, L₂, Y, R₃ₐ, R_{3b}, and q are as described above, or a pharmaceutically acceptable salt thereof.

In certain embodiments, the present invention provides a compound of Formula Ia:

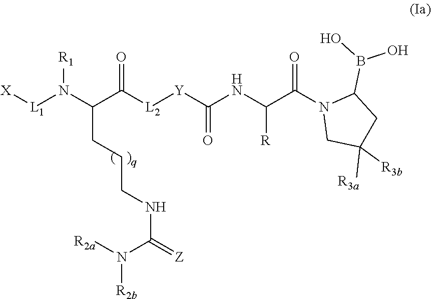

wherein:
X is a chelator suitable for radiolabeling, or a chelator bound to a radioactive atom or complex;
L₁ is a bond, or a group of the formula:

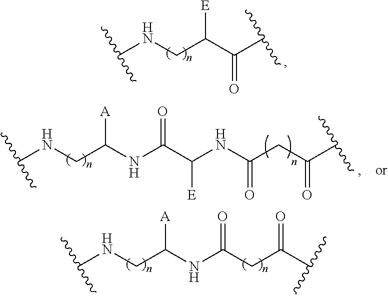

wherein each n is independently 0, 1, 2, 3, 4, or 5;
q is 0, 1, or 2;
Z is ═O or ═NR_{2c};
A is —H or —COOH;
E is —H,

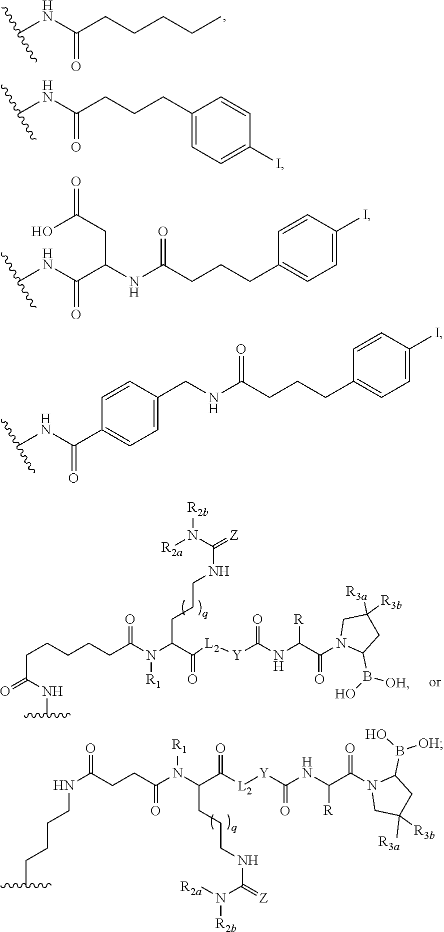

wherein
each R is independently —H, —CH₃, or —CH₂OH;
R₁ is —H or —C₁₋₃ alkyl;
R₂ₐ, R_{2b} and R_{2c} are each independently —H or —C₁₋₃ alkyl;
R₃ₐ and R_{3b} are each independently —H, —OH, or halogen; and
L₂ is a group of the formula:

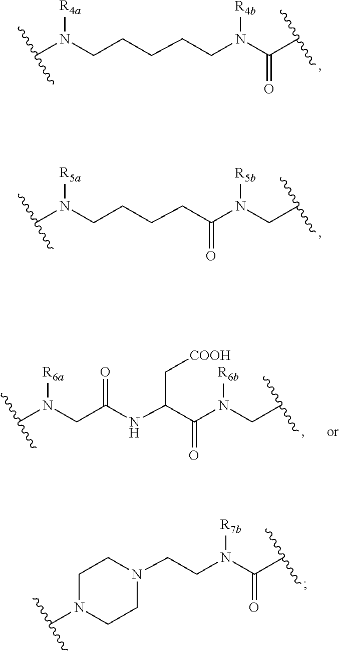

wherein R₄ₐ and R_{4b} are each independently —H or —C₁₋₃ alkyl;
R₅ₐ and R_{5b} are each independently —H or —C₁₋₃ alkyl;
R₆ₐ and R_{6b} are each independently —H or —C₁₋₃ alkyl; and
R₇ₐ is —FH or —C₁₋₃ alkyl;
Y is selected from the group consisting of

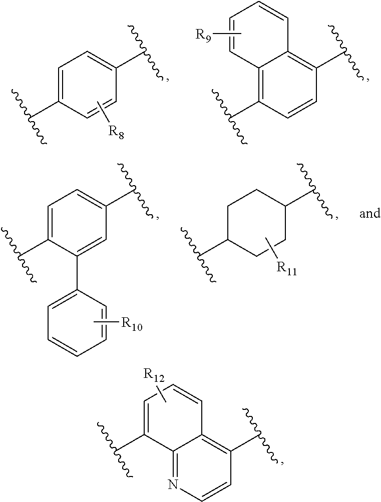

wherein R₈ is —H, —OH, halogen, —OR₈ₐ, —NR_{8b}R_{8c}, —COOH, or a 5 to 6 membered heteroaryl;
R₉ is —H, —OH, halogen, —OR₉ₐ, —NR_{9b}R_{9c}, or —COOH;
R₁₀ is —H, —OH, halogen, —OR₁₀ₐ, —NR_{10b}R_{10c}, or —COOH; and
R₁₁ is —H, —OH, halogen, —OR₁₁ₐ, —NR_{11b}R_{11c}, or —COOH;
wherein R₈ₐ, R_{8b}, and R_{8c} are each independently —H or —C₁₋₃ alkyl;
R₉ₐ, R_{9b}, and R_{9c} are each independently —H or —C₁₋₃ alkyl;
R₁₀ₐ, R_{10b}, and R_{10c} are each independently —H or —C₁₋₃ alkyl;
R₁₁ₐ, R_{11b}, and R_{11c} are each independently —H or —C₁₋₃ alkyl; and
R₁₂ is —H, —OH, halogen, —OR₉ₐ, —NR_{9b}R_{9c}, or —COOH;
or a pharmaceutically acceptable salt thereof.

As used herein, the term “halogen” means fluoro (F), chloro (Cl), bromo (Br), or iodo (I).

As used herein, the term “C₁₋₃ alkyl” means saturated linear or branched-chain monovalent hydrocarbon radicals of one to three carbon atoms. Examples of C₁₋₃ alkyls include methyl, ethyl, propyl, 1-propyl, and isopropyl.

As used herein, the term “megabecquerel” (MBq) refers to a unit of radioactivity measurement in the International System of Units (SI), where 1 MBq=1,000,000 becquerels (Bq). A becquerel (Bq) is defined as one radioactive decay event per second. Doses of radiopharmaceuticals are commonly expressed in MBq to quantify the level of radioactivity administered to a patient.

The term “heteroatom” refers to nitrogen, oxygen, or sulfur, and includes any oxidized form of nitrogen or sulfur, and any quaternary form of a basic nitrogen.

As used herein, the term “5 to 6 membered heteroaryl” refers to groups having 5 to 6 ring atoms, and having, in addition to carbon atoms, from one to five heteroatoms. Heteroaryl groups include, without limitation, thienyl, furanyl, pyrrolyl, imidazolyl pyrazolyl, triazolyl, tetrazolyl, oxazolyl, isoxazolyl, oxadiazolyl, thiazolyl, isothiazolyl, thiadiazolyl pyridyl, pyridazinyl, pyrimidinyl, and pyrazinyl.

In an embodiment of a compound of Formula I or a pharmaceutically acceptable salt thereof, of formula (IIa):

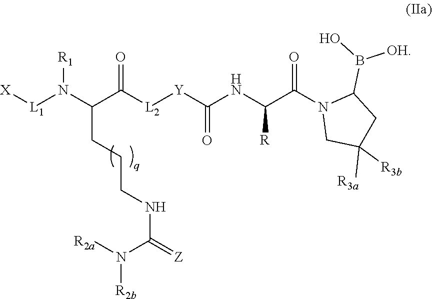

In an embodiment of a compound of Formula I or a pharmaceutically acceptable salt thereof, of formula (IIb):

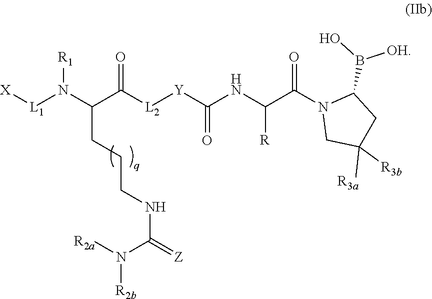

In an embodiment of a compound of Formula I or a pharmaceutically acceptable salt thereof, of formula (IIc):

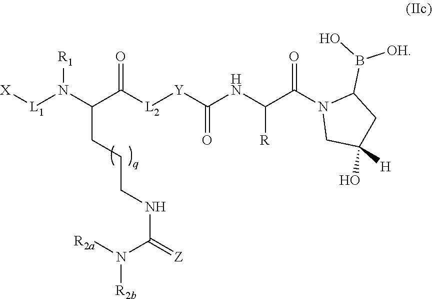

In an embodiment of a compound of Formula I or a pharmaceutically acceptable salt thereof, of formula (IId):

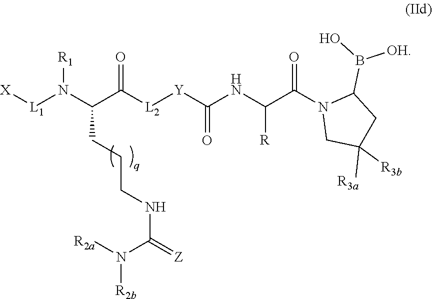

In an embodiment of a compound of Formula I or a pharmaceutically acceptable salt thereof, of formula (IIe):

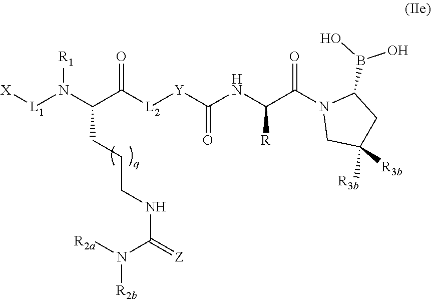

In an embodiment of Formula I or a pharmaceutically acceptable salt thereof, or an embodiment of any one of Formulae IIa-IIe or a pharmaceutically acceptable salt thereof, each R is H.

In an embodiment of Formula I or a pharmaceutically acceptable salt thereof, or an embodiment of any one of Formulae IIa-IIe or a pharmaceutically acceptable salt thereof, each R is methyl.

In an embodiment of Formula I or a pharmaceutically acceptable salt thereof, or an embodiment of any one of Formulae IIa-IIe or a pharmaceutically acceptable salt thereof, each R is —CH₂OH.

In an embodiment of a compound of Formula I or a pharmaceutically acceptable salt thereof, of formula (IIIa):

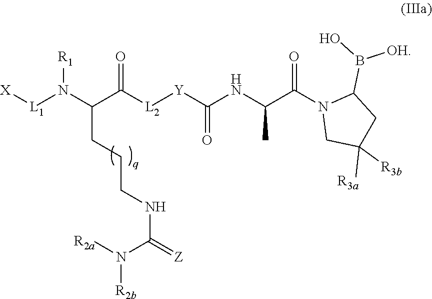

In an embodiment of a compound of Formula I or a pharmaceutically acceptable salt thereof, of formula (IIIb):

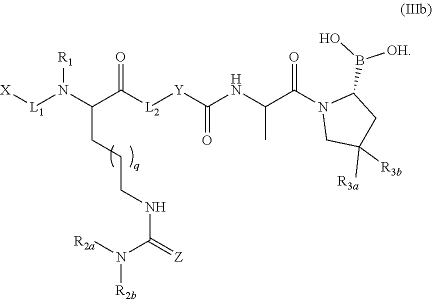

In an embodiment of a compound of Formula I or a pharmaceutically acceptable salt thereof, of formula (IIIc):

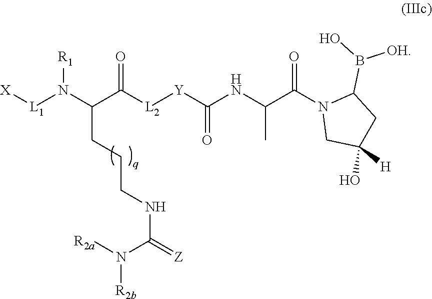

In an embodiment of a compound of Formula I or a pharmaceutically acceptable salt thereof, of formula (IIId):

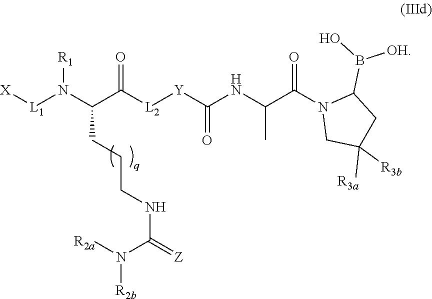

In an embodiment of a compound of Formula I or a pharmaceutically acceptable salt thereof, of formula (Ille):

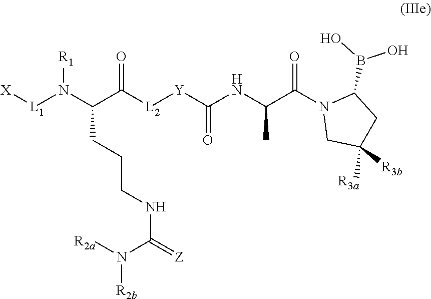

As used herein, a compound of formula I or a pharmaceutically acceptable salt thereof, includes a compound of formula IIa, IIb, IIc, IId, IIe, IIIa, IIIb, IIIc, IIId, or IIIe, or a pharmaceutically acceptable salt thereof.

In an embodiment of a compound of Formula I or a pharmaceutically acceptable salt thereof, L₁ is selected from the group consisting of a bond, or a group of the formula:

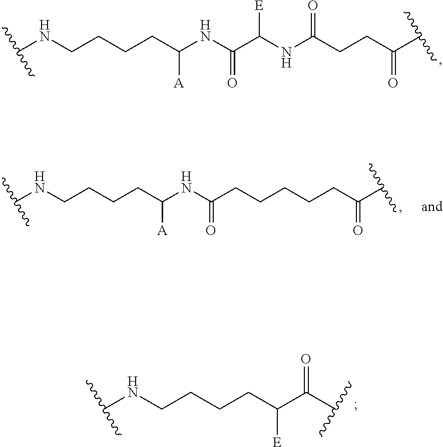

wherein A is —H or —COOH.

In an embodiment of a compound of Formula I or a pharmaceutically acceptable salt thereof, L₁ is selected from the group consisting of a bond, or a group of the formula:

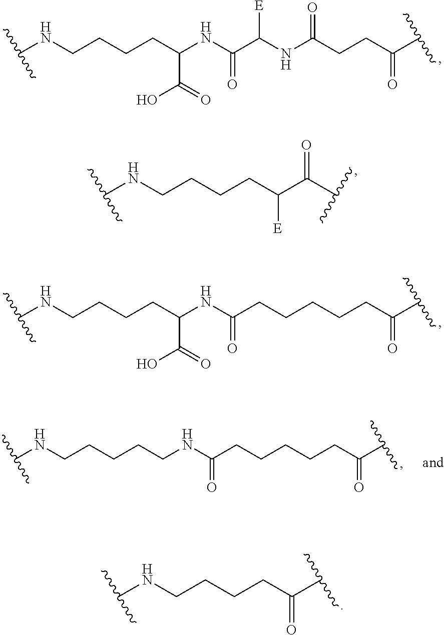

In an embodiment of a compound of Formula I or a pharmaceutically acceptable salt thereof, E is selected from the group consisting of:

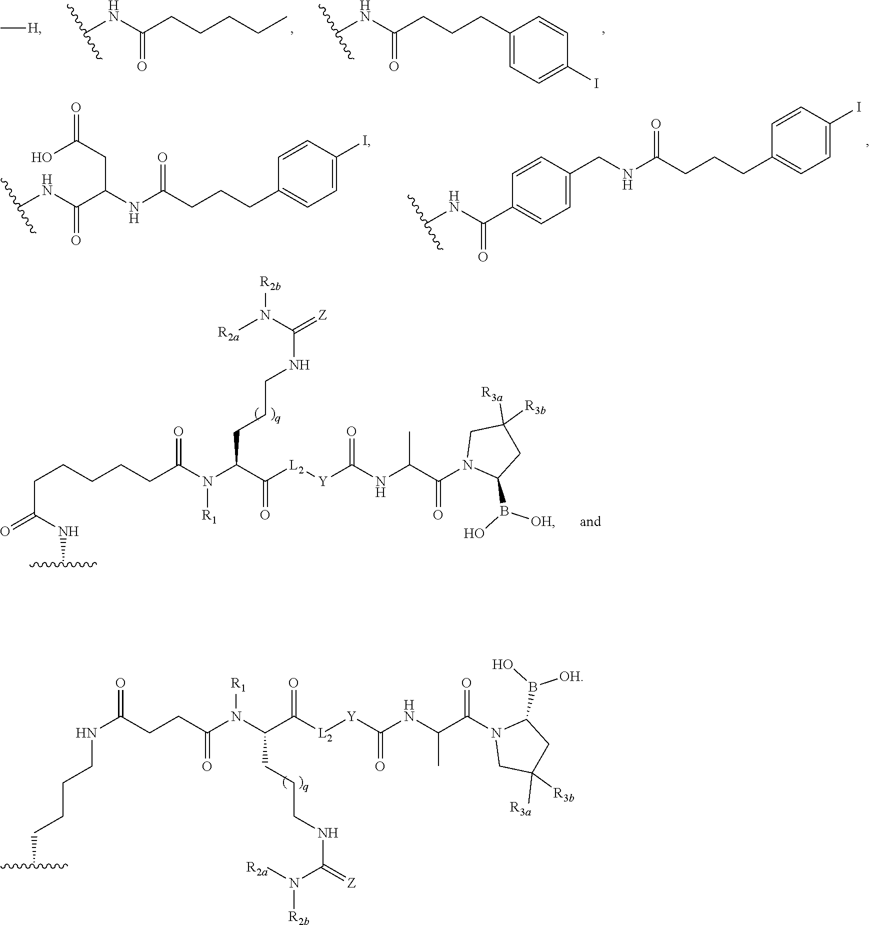

In an embodiment of a compound of Formula I or a pharmaceutically acceptable salt thereof, E is selected from the group consisting of:

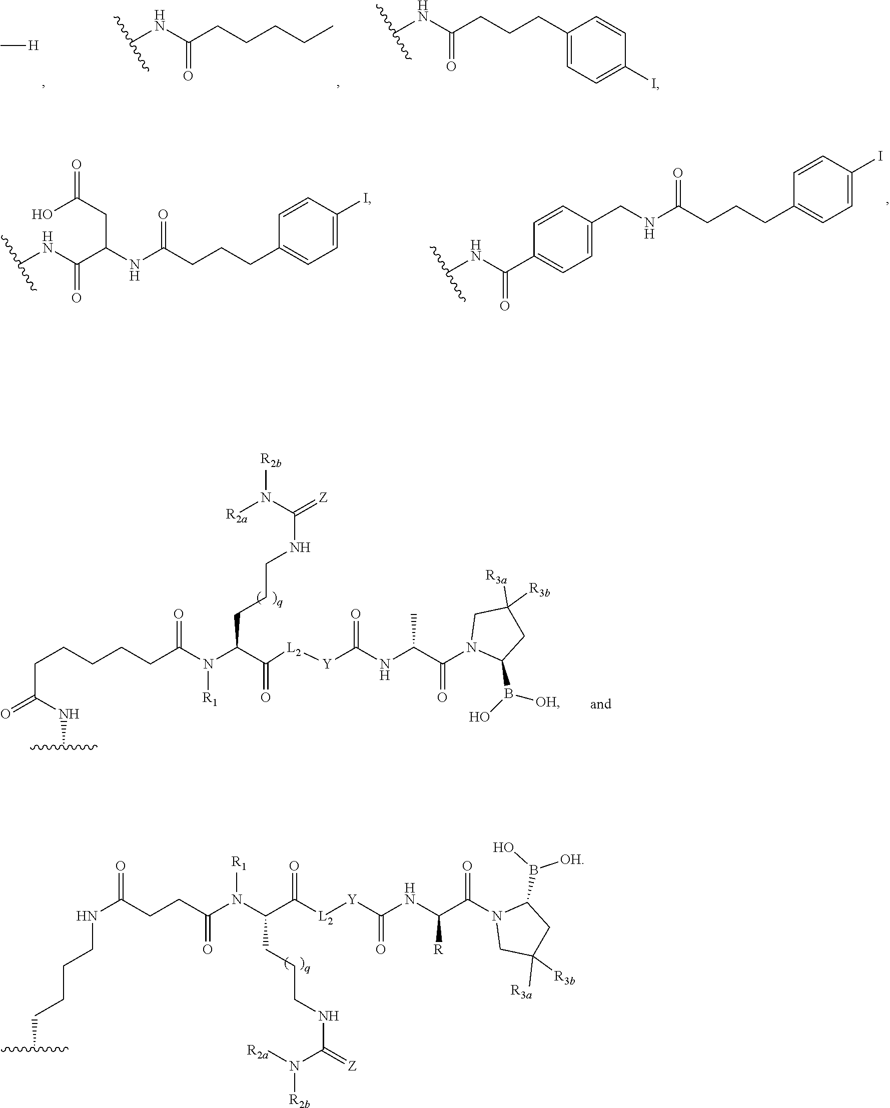

In an embodiment of a compound of Formula I or a pharmaceutically acceptable salt thereof, E is selected from the group consisting of:

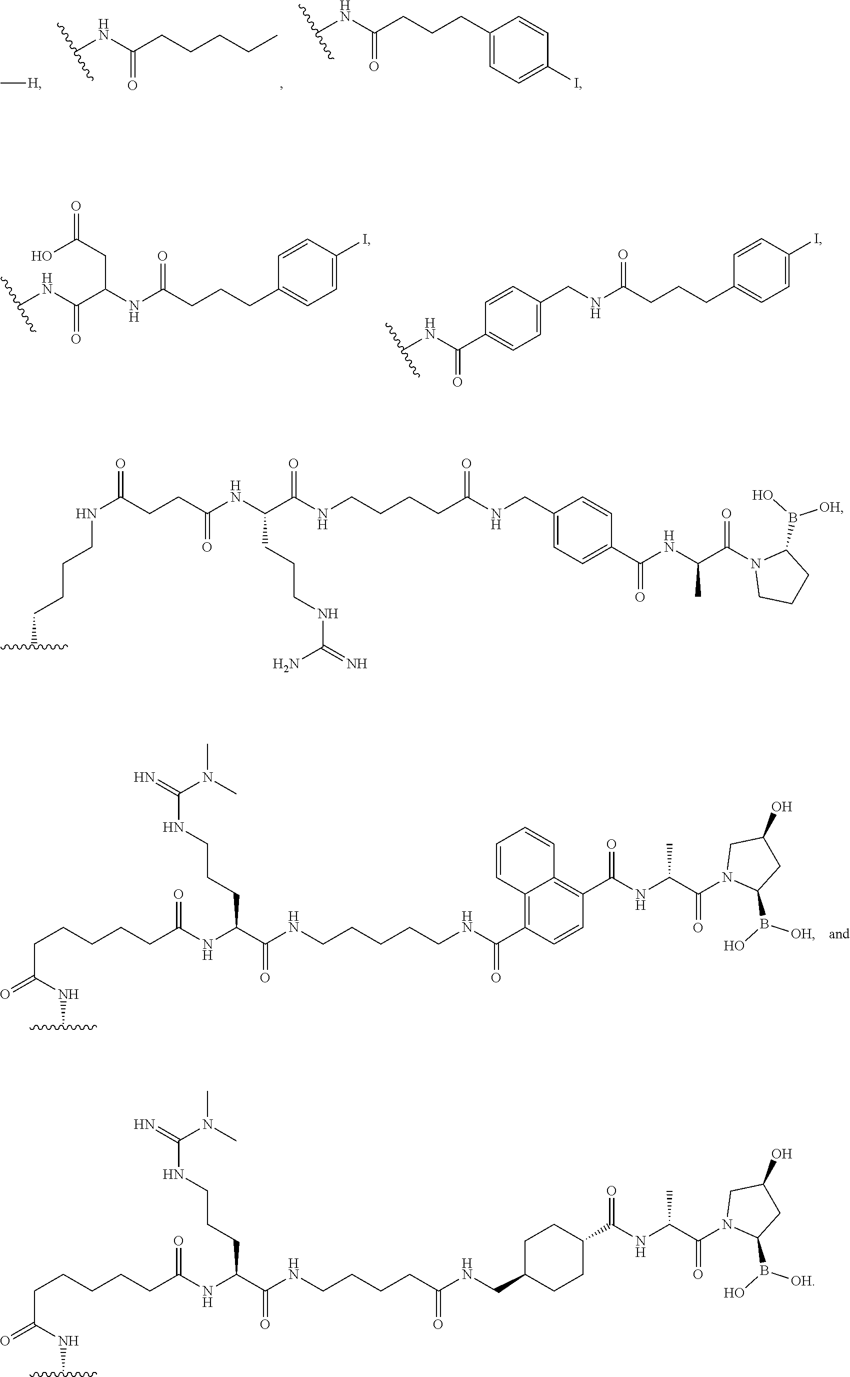

In an embodiment of a compound of Formula I or a pharmaceutically acceptable salt thereof, L₂ is selected from the group consisting of:

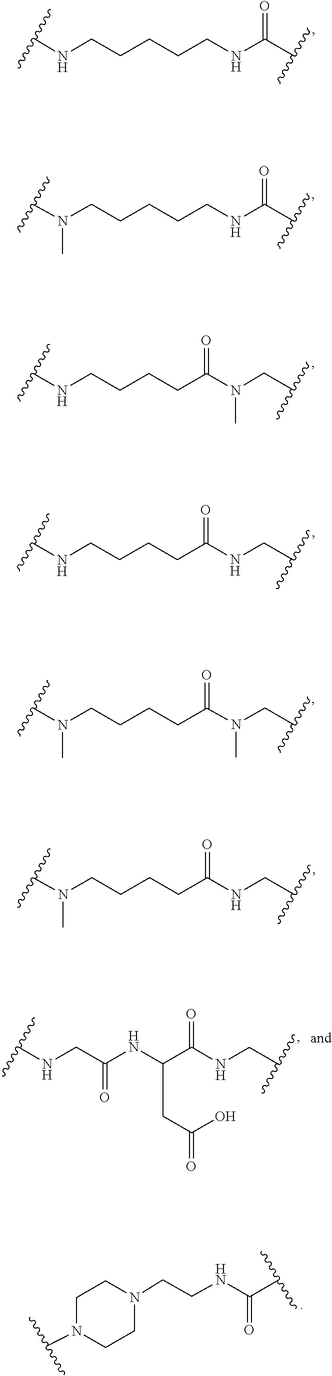

In a further embodiment of a compound of Formula I or a pharmaceutically acceptable salt thereof, L₂ is selected from the group consisting of:

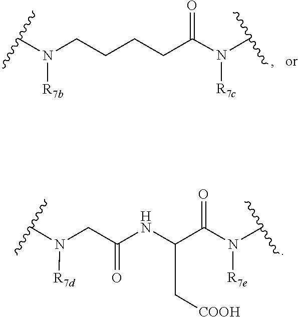

wherein, in these embodiments, R_{7b} and R_{7c} are each independently selected from —H or —C₁₋₃ alkyl; and
R_{7d} and R₇ₑ are each independently selected from —H or —C₁₋₃ alkyl.

In a further embodiment of a compound of Formula I or a pharmaceutically acceptable salt thereof, L₂ is selected from the group consisting of:

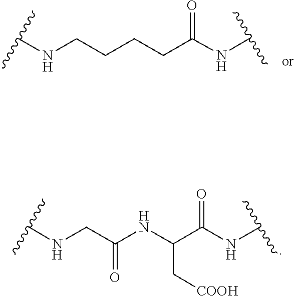 [SMILES: *NCCCCC(=O)N* ; *NCC(=O)NC(CC(=O)O)C(=O)N*]

In an embodiment of a compound of Formula I or a pharmaceutically acceptable salt thereof, R₈ is —H, —F, —OCH₃, —COOH, or

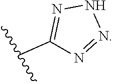 [SMILES: *c1nn[nH]n1]

In an embodiment of a compound of Formula I or a pharmaceutically acceptable salt thereof, R₉ is —H, —OCH₃, or —N(CH₃)₂.

In an embodiment of a compound of Formula I or a pharmaceutically acceptable salt thereof, R₁₀ is —H, —Cl, —OH, or —N(CH₃)₂.

In an embodiment of a compound of Formula I or a pharmaceutically acceptable salt thereof, Z is ═O or ═NH, preferably Z is ═NH.

In an embodiment of a compound of Formula I or a pharmaceutically acceptable salt thereof, Y is selected from the group consisting of:

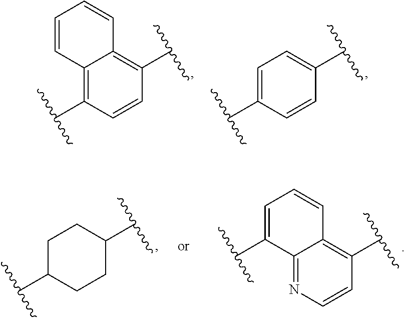 [SMILES: *c1ccc(*)c2ccccc12 ; *c1ccc(*)cc1 ; *C1CCC(*)CC1 ; *c1ccnc2c(*)cccc12]

In an embodiment of a compound of Formula I or a pharmaceutically acceptable salt thereof, X is a chelator suitable for radiolabeling selected from the group consisting of:

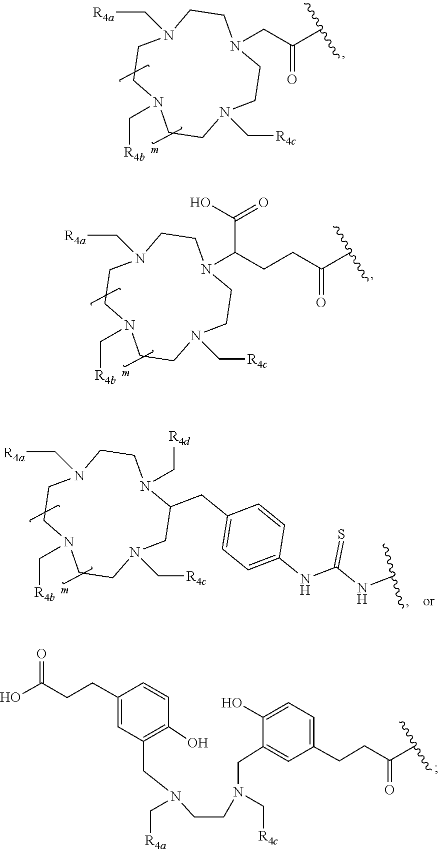

wherein m is 0, 1, or 2;
R₄ₐ is independently —H, —COOH, or —CONH₂;
R_{4b} is independently —H, —COOH, or —CONH₂; and
R_{4c} is independently —H, —COOH, or —CONH₂.

In an embodiment of a compound of Formula I or a pharmaceutically acceptable salt thereof, X is a chelator suitable for radiolabeling selected from the group consisting of:

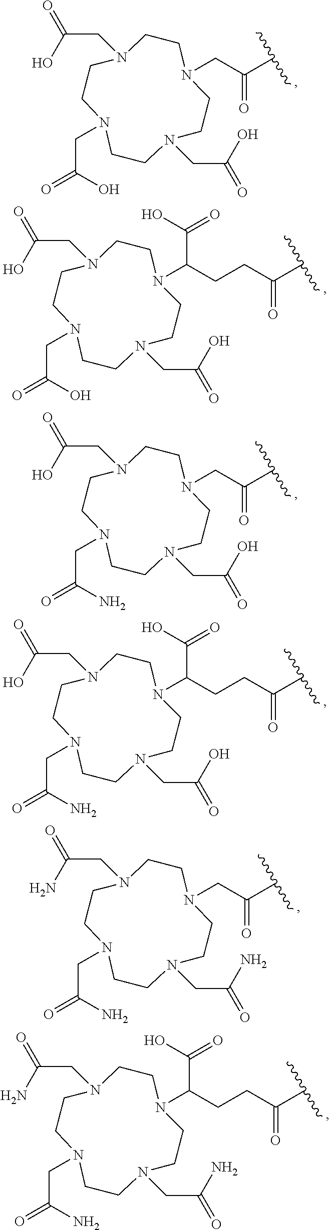

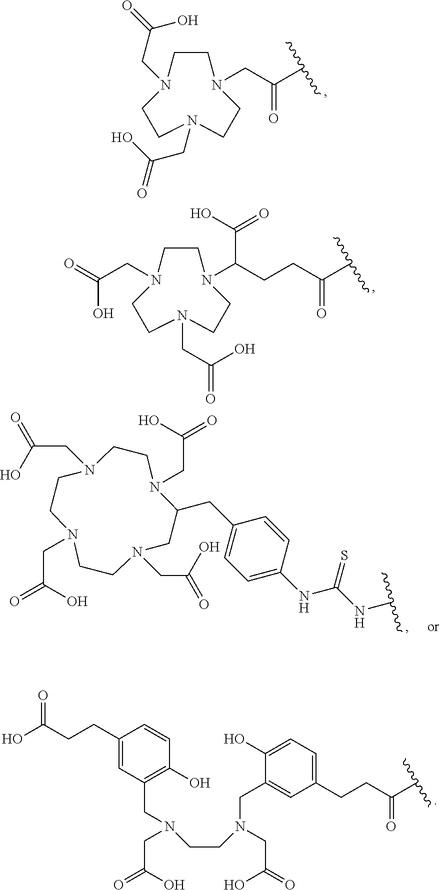

In an embodiment of a compound of Formula I or a pharmaceutically acceptable salt thereof, X is a chelator suitable for radiolabeling of the formula:

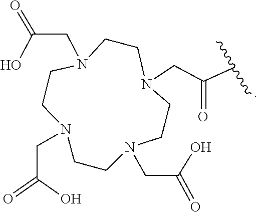 [SMILES: *C(=O)CN1CCN(CC(=O)O)CCN(CC(=O)O)CCN(CC(=O)O)CC1]

In an embodiment of a compound of Formula I or a pharmaceutically acceptable salt thereof, X is a chelator suitable for radiolabeling of the formula:

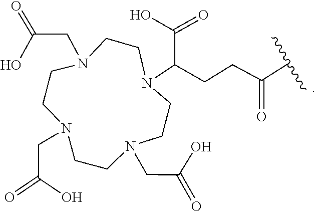 [SMILES: *C(=O)CCC(C(=O)O)N1CCN(CC(=O)O)CCN(CC(=O)O)CCN(CC(=O)O)CC1]

In an embodiment of a compound of Formula I or a pharmaceutically acceptable salt thereof, X is a chelator suitable for radiolabeling of the formula:

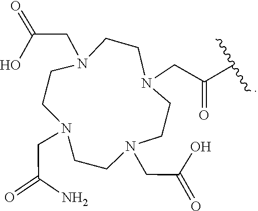 [SMILES: *C(=O)CN1CCN(CC(=O)O)CCN(CC(N)=O)CCN(CC(=O)O)CC1]

In an embodiment of a compound of Formula I or a pharmaceutically acceptable salt thereof, wherein X is a chelator bound to a radioactive atom and the radioactive atom is selected from the group consisting of: ²²³Ra, ⁸⁹Sr, ^{94m}Tc, ^{99m}Tc, ¹⁸⁶Re, ¹⁸⁸Re, ²⁰³Pb, ²¹²Pb, ⁶⁷Ga, ⁶⁸Ga, ⁴⁷Sc, ¹¹¹In, ⁹⁷Ru, ⁶²Cu, ⁶⁴Cu, ⁸⁶Y, ⁸⁸Y, ⁸⁹Zr, ⁹⁰Y, ¹²¹Sn, ¹⁶¹Tb, ¹⁵³Sm, ¹⁶⁶Ho, ¹⁰⁵Rh, ¹⁷⁷Lu, ¹²³I, ¹²⁴I, ¹²⁵I, ¹³¹I, ¹⁸F, ²¹¹At, ²²⁵Ac, ⁸⁹Sr, ^{117m}Sn, ¹⁶⁹Er, or [¹⁸F]AlF.

In an embodiment of a compound of Formula I or a pharmaceutically acceptable salt thereof, wherein X is a chelator bound to a radioactive atom and the radioactive atom is selected from the group consisting of: ²²⁵Ac, ²¹¹At, ⁶⁴Cu, ⁶⁷Cu, ¹⁸F, [¹⁸F]AlF, ⁶⁷Ga, ⁶⁸Ga, ¹¹¹In, ¹⁷⁷Lu, ²⁰³Pb, ²¹²Pb, ¹⁶¹Tb, ⁸⁶Y, ⁹⁰Y, or ⁸⁹Zr.

In an embodiment of a compound of Formula I or a pharmaceutically acceptable salt thereof, the radioactive atom is ¹⁷⁷Lu.

In an embodiment of a compound of Formula I or a pharmaceutically acceptable salt thereof, the radioactive atom is ²¹²Pb.

In an embodiment of a compound of Formula I or a pharmaceutically acceptable salt thereof, the radioactive atom is [¹⁸F]AlF.

In an embodiment of a compound of Formula I or a pharmaceutically acceptable salt thereof, the radioactive atom is ²²⁵Ac.

In an embodiment of a compound of Formula I or a pharmaceutically acceptable salt thereof, the radioactive atom is ¹⁶¹Tb.

In an embodiment of a compound of Formula I or a pharmaceutically acceptable salt thereof, wherein X is a chelator bound to a radioactive atom selected from the group consisting of:

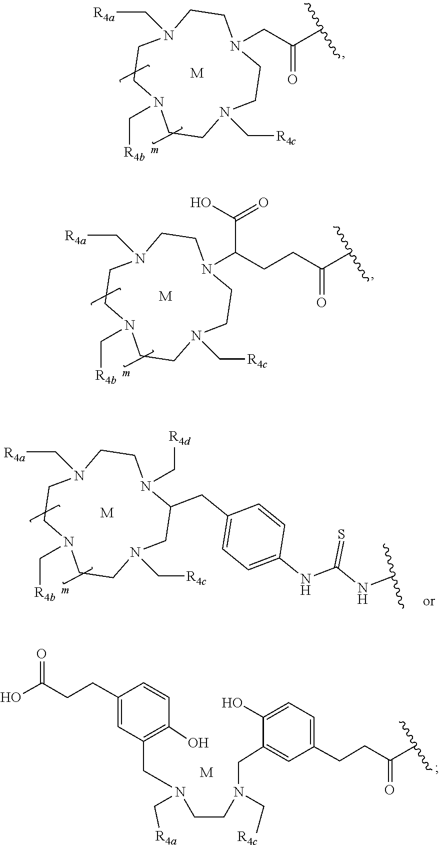

wherein M is the radioactive atom,
m is 0, 1, or 2;
R₄ₐ is independently —H, —COOH, or —CONH₂;
R_{4b} is independently —H, —COOH, or —CONH₂;
R_{4c} is independently —H, —COOH, or —CONH₂; and
R_{4d} is independently —H, —COOH, or —CONH₂.

In an embodiment of a compound of Formula I or a pharmaceutically acceptable salt thereof, wherein X is a chelator bound to a radioactive atom selected from the group consisting of:

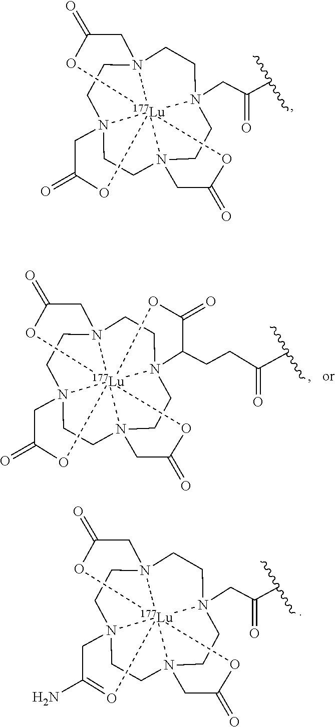

In an embodiment of a compound of Formula I or a pharmaceutically acceptable salt thereof, wherein X is a chelator bound to a radioactive atom selected from the group consisting of:

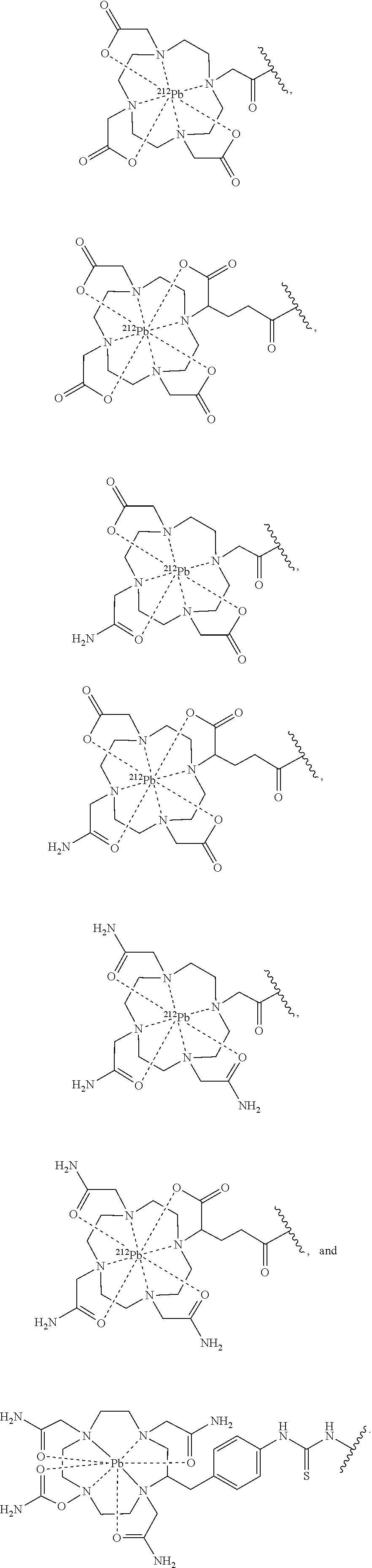

In an embodiment of a compound of Formula I or a pharmaceutically acceptable salt thereof, wherein X is a chelator bound to a radioactive atom selected from the group consisting of:

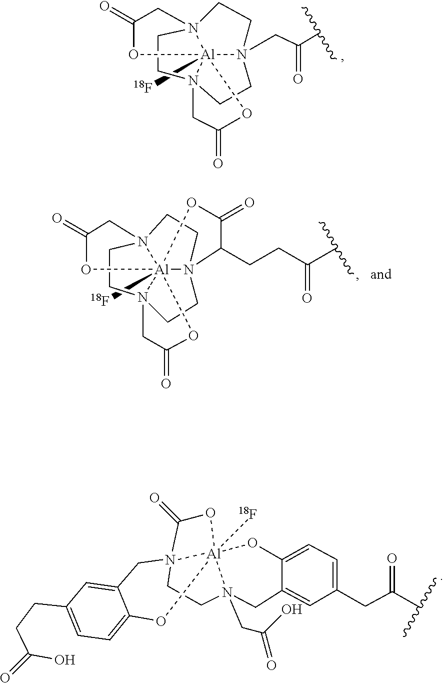

In an embodiment, a compound of Formula I or a pharmaceutically acceptable salt thereof, is selected from the group consisting of:

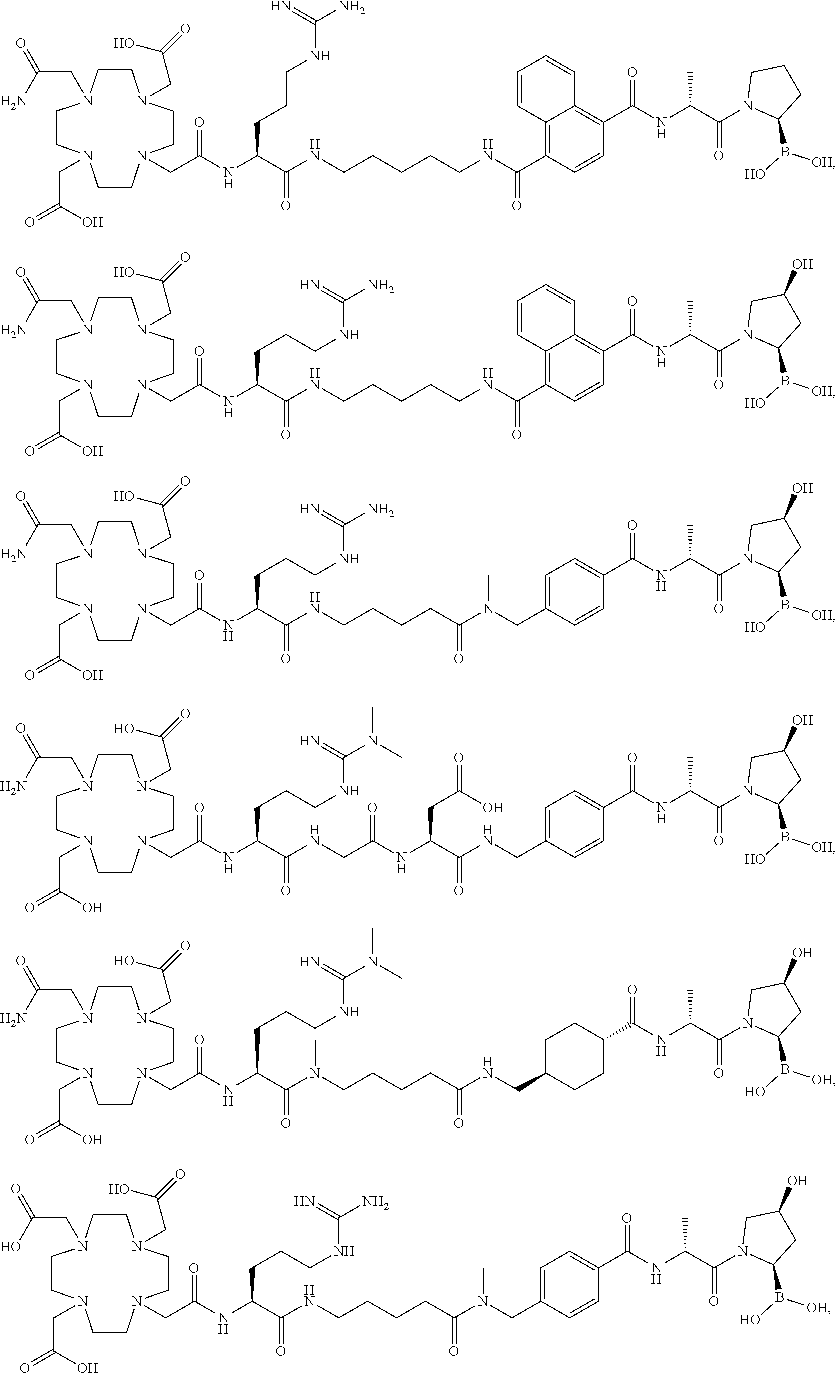

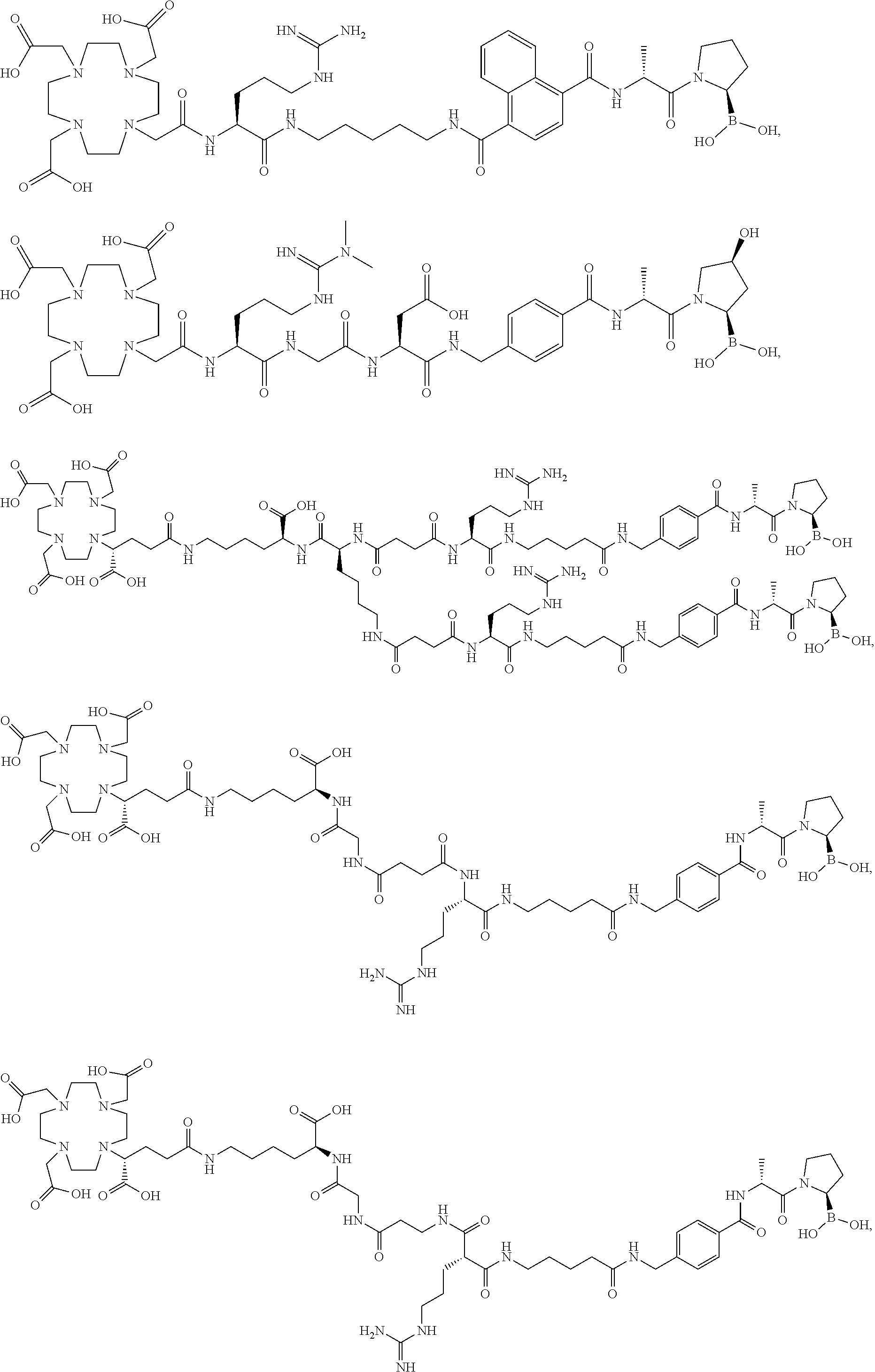

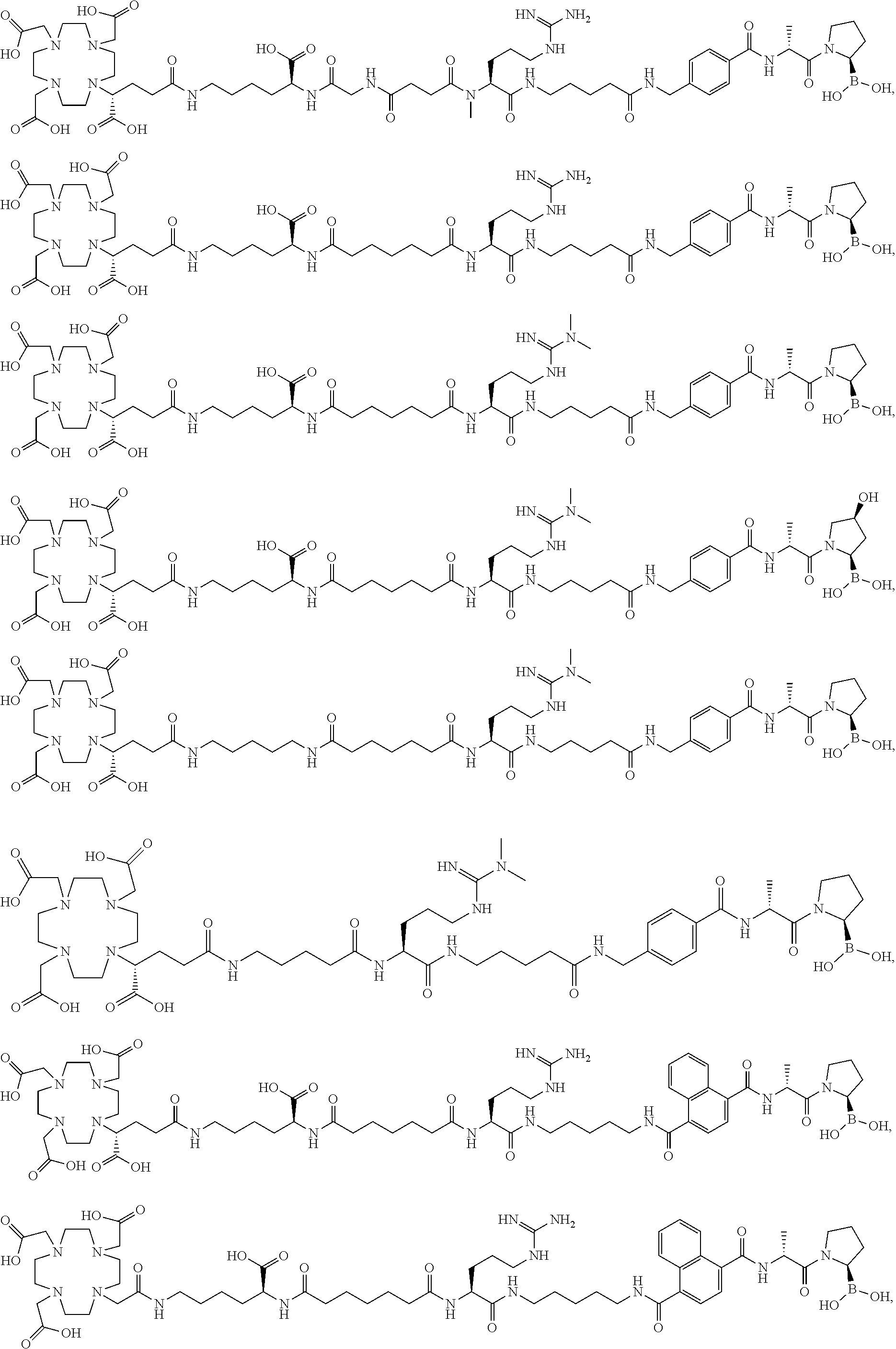

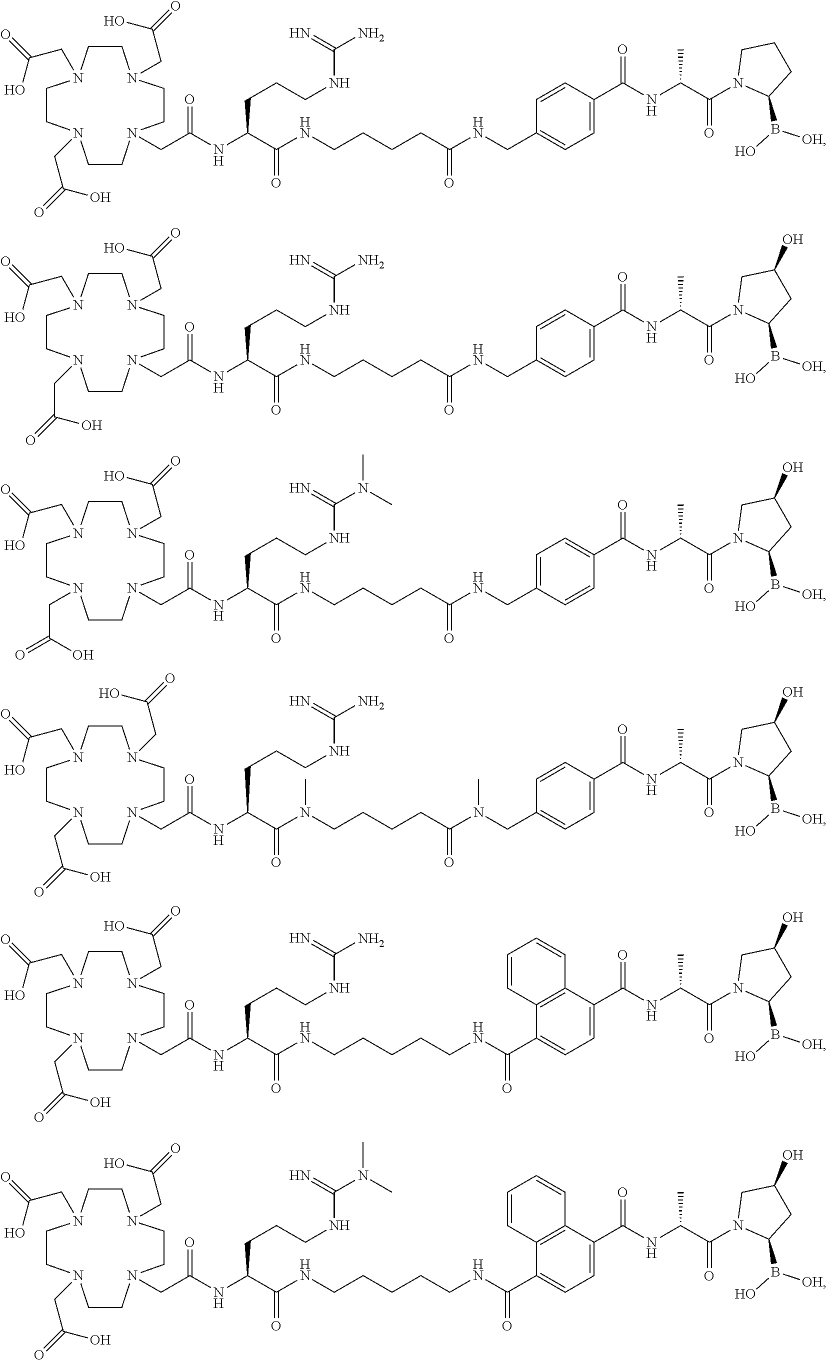

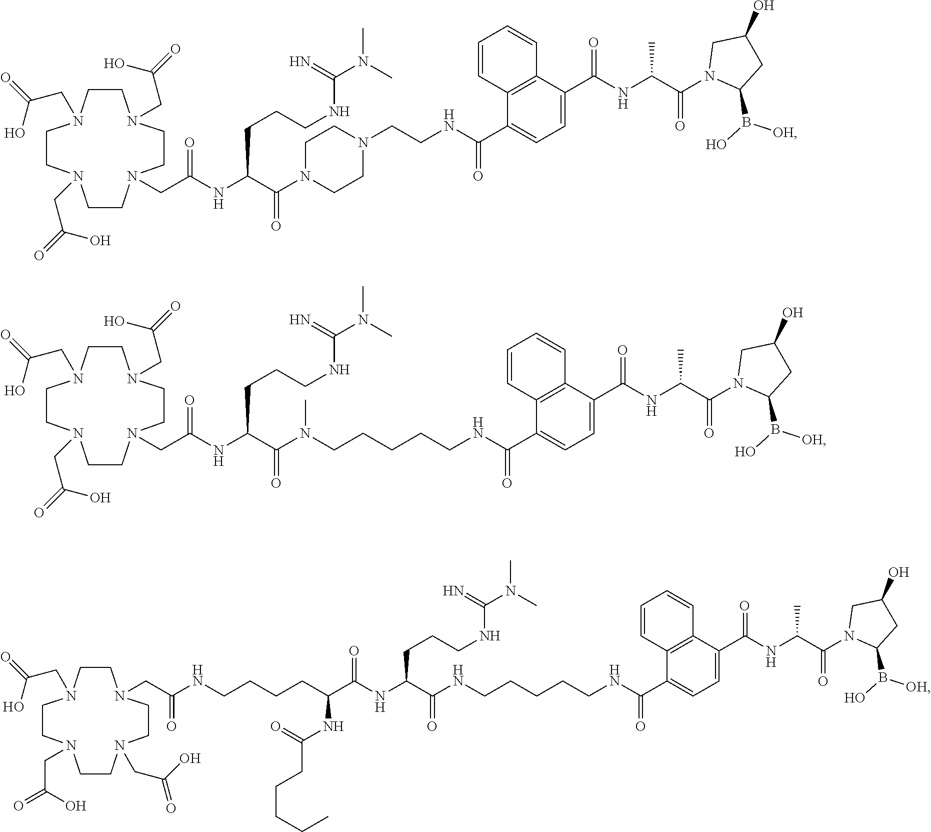 [SMILES: C[C@@H](NC(=O)c1ccc(C(=O)NCCN2CCN(C(=O)[C@H](CCCNC(=N)N(C)C)NC(=O)CN3CCN(CC(=O)O)CCN(CC(=O)O)CCN(CC(=O)O)CC3)CC2)c2ccccc12)C(=O)N1C[C@@H](O)C[C@H]1B(O)O ; C[C@@H](NC(=O)c1ccc(C(=O)NCCCCCN(C)C(=O)[C@H](CCCNC(=N)N(C)C)NC(=O)CN2CCN(CC(=O)O)CCN(CC(=O)O)CCN(CC(=O)O)CC2)c2ccccc12)C(=O)N1C[C@@H](O)C[C@H]1B(O)O ; CCCCCC(=O)N[C@@H](CCCCNC(=O)CN1CCN(CC(=O)O)CCN(CC(=O)O)CCN(CC(=O)O)CC1)C(=O)N[C@@H](CCCNC(=N)N(C)C)C(=O)NCCCCCNC(=O)c1ccc(C(=O)N[C@H](C)C(=O)N2C[C@@H](O)C[C@H]2B(O)O)c2ccccc12]

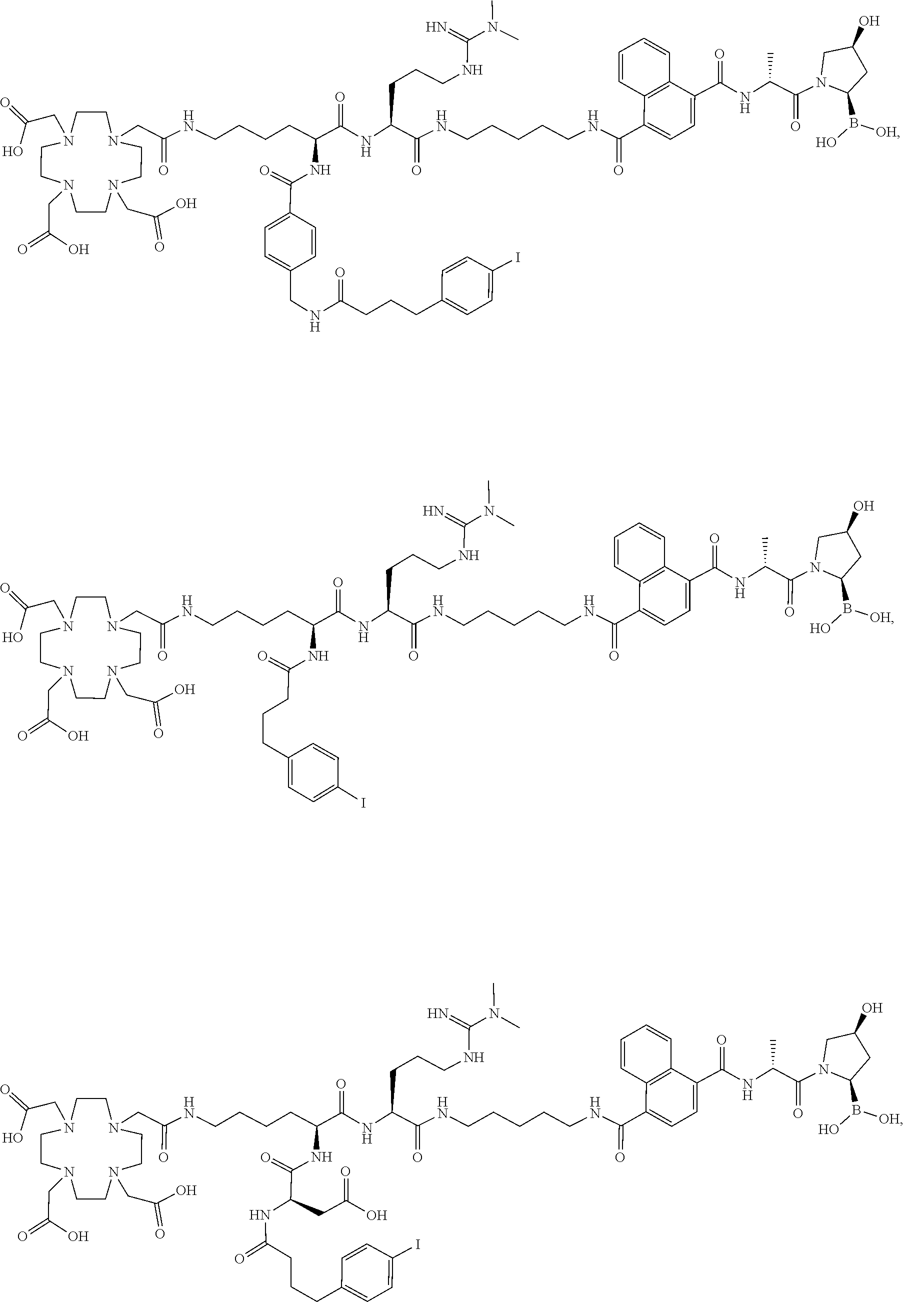

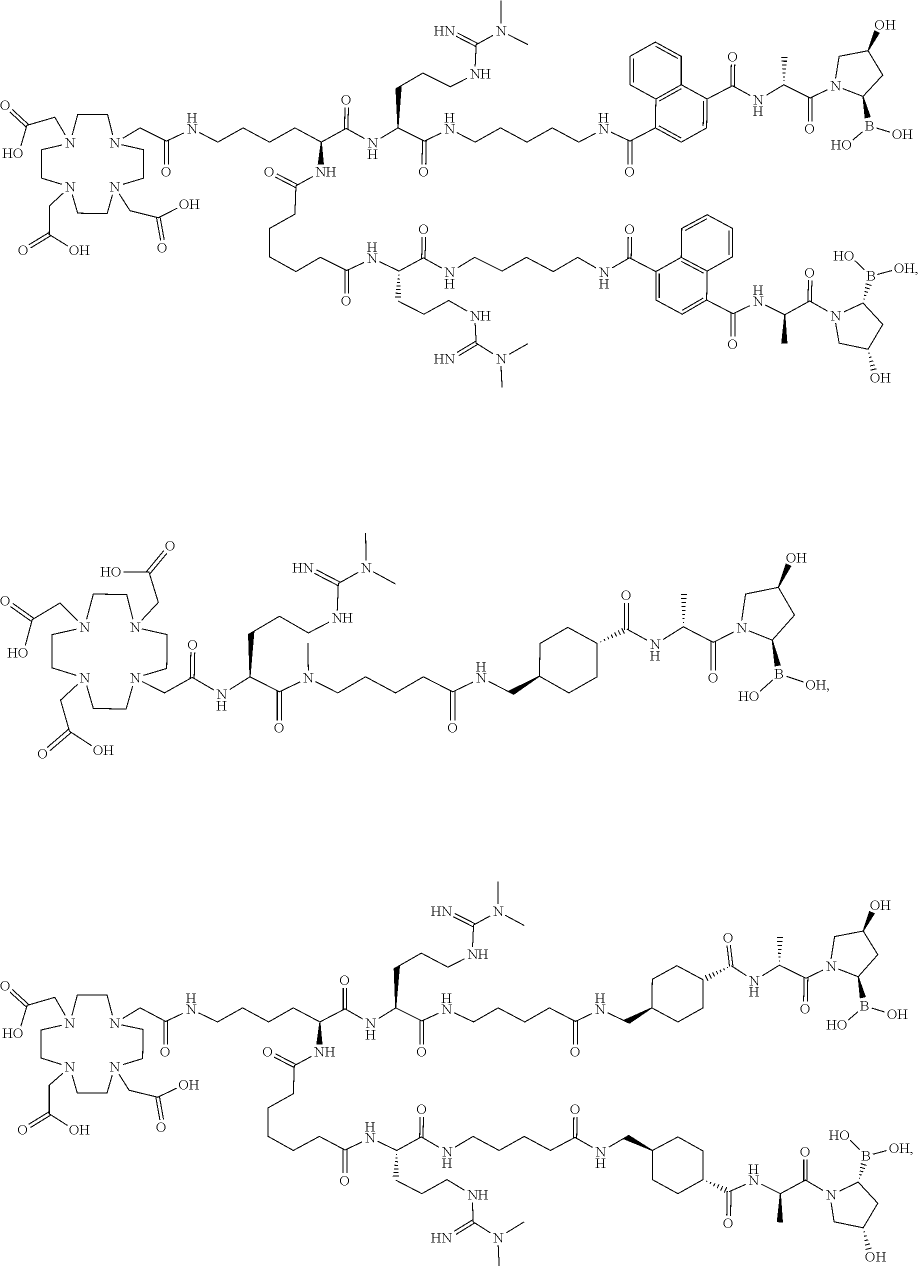

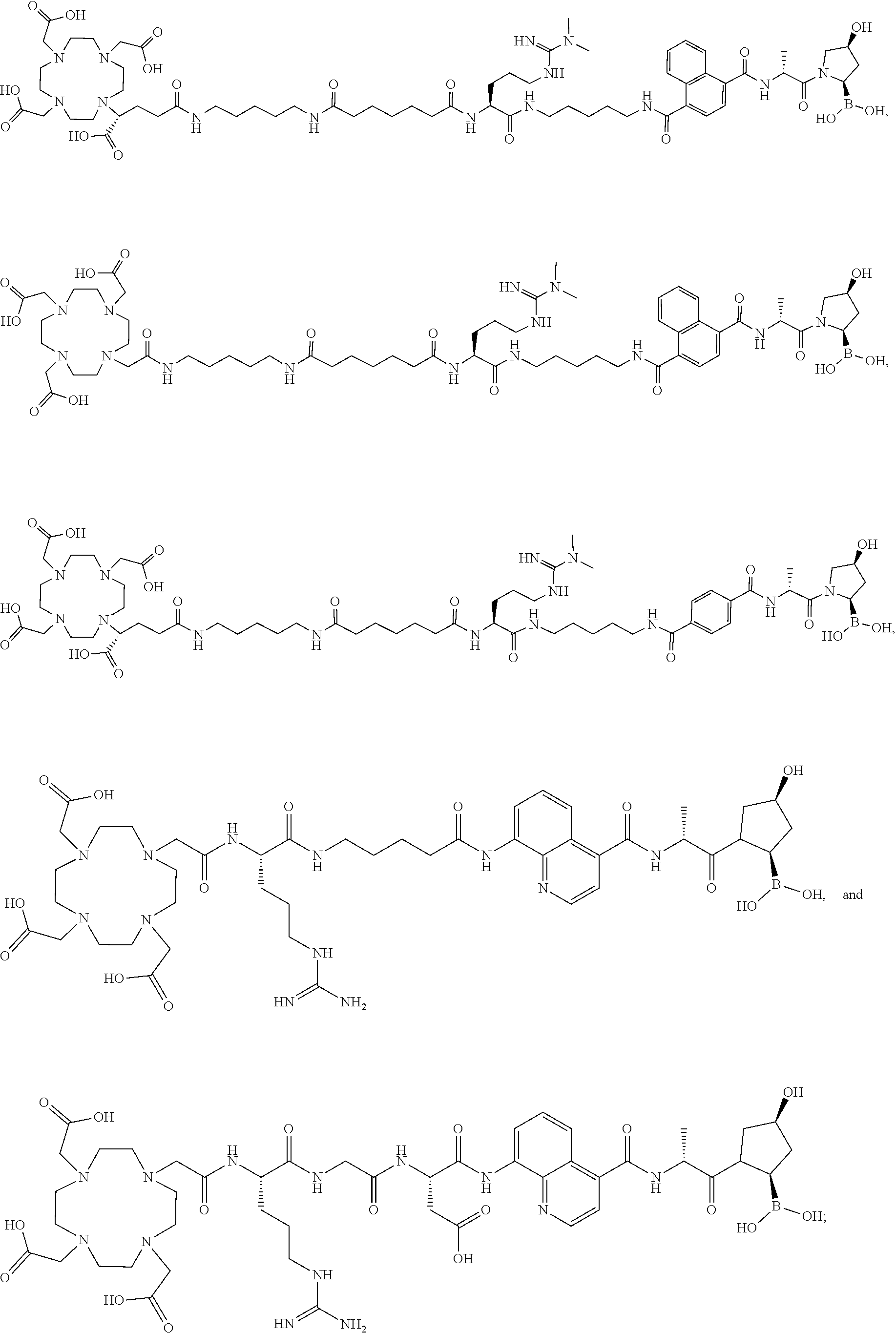

or a pharmaceutically acceptable salt thereof.

In further embodiments, a compound of Formula I or a pharmaceutically acceptable salt thereof, is selected from the group consisting of:

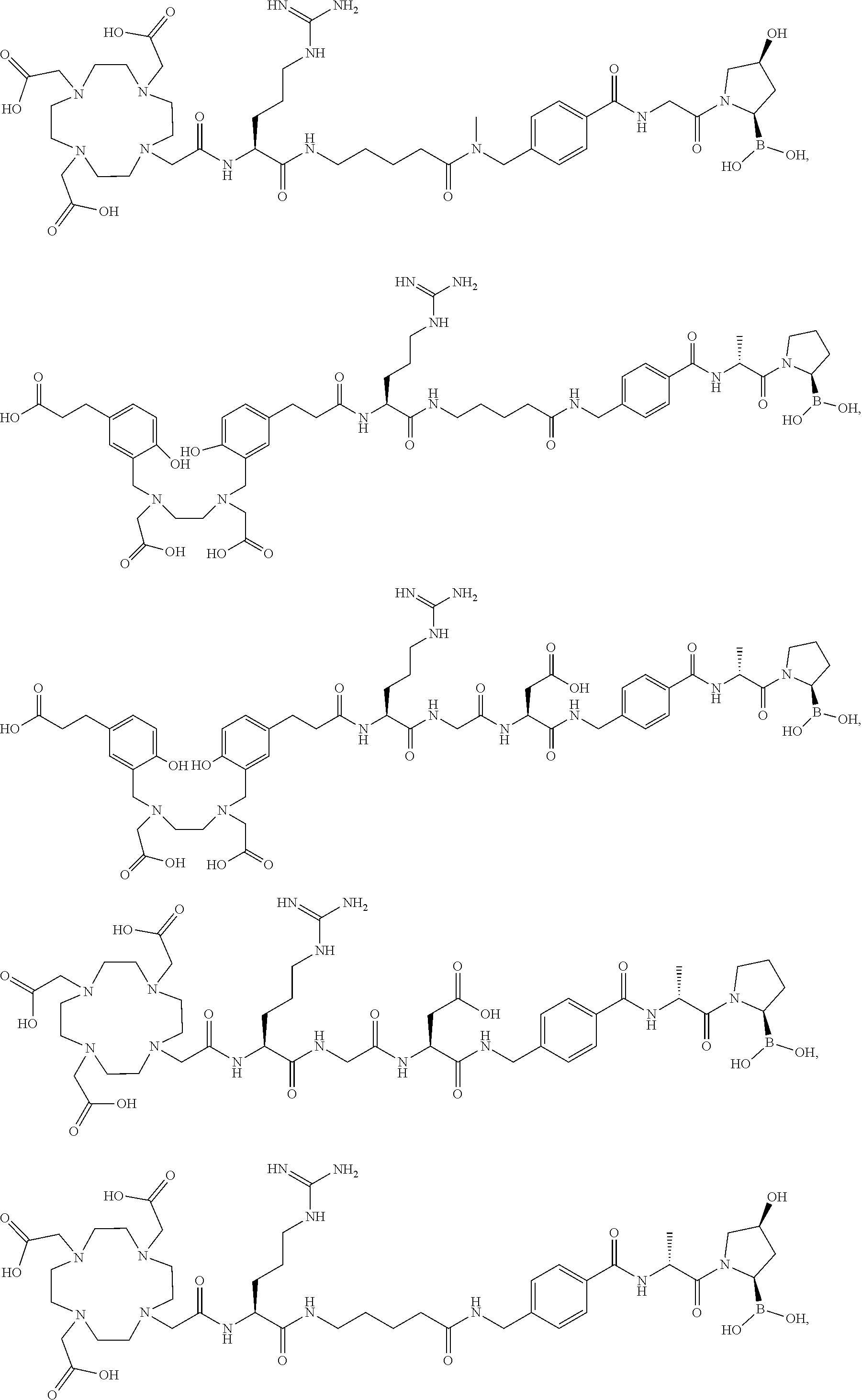

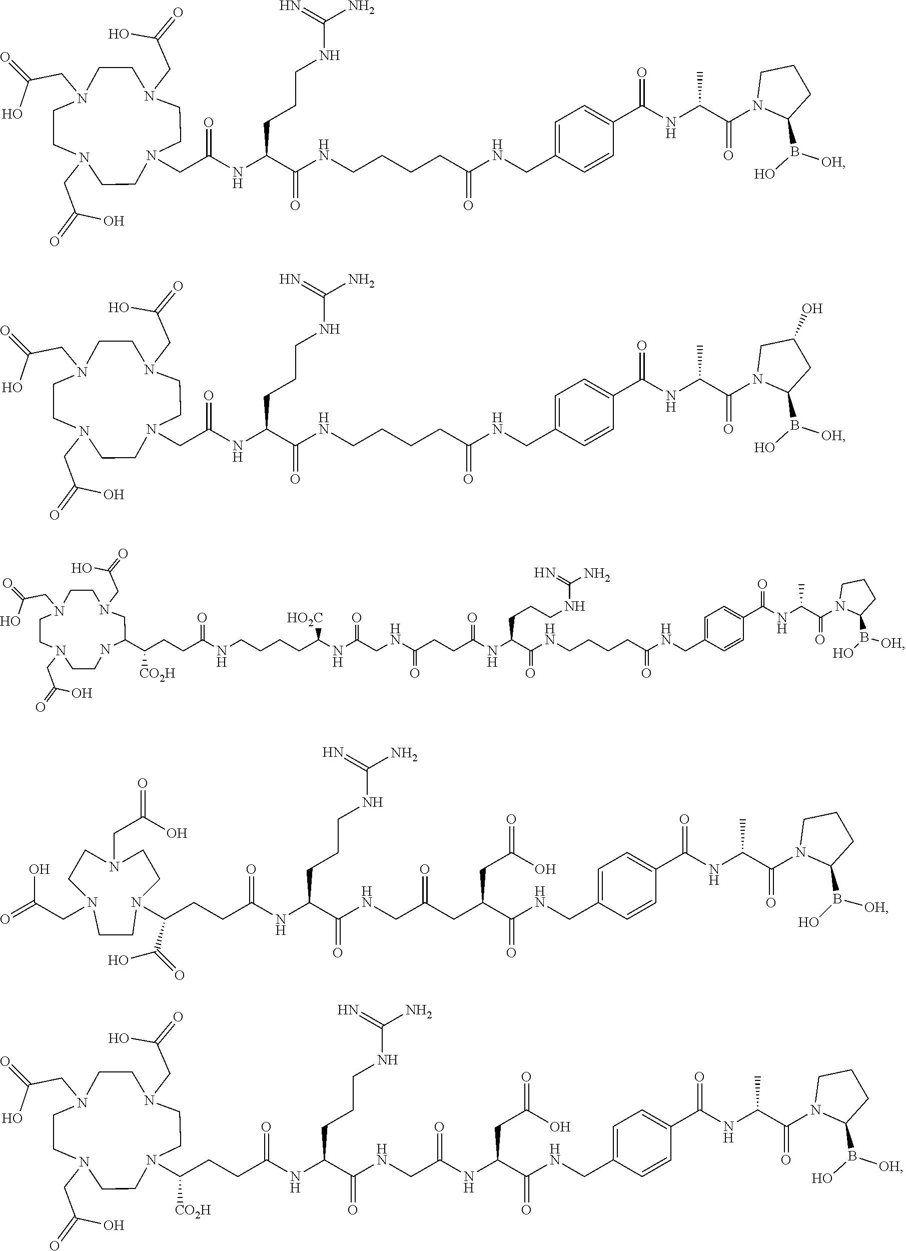

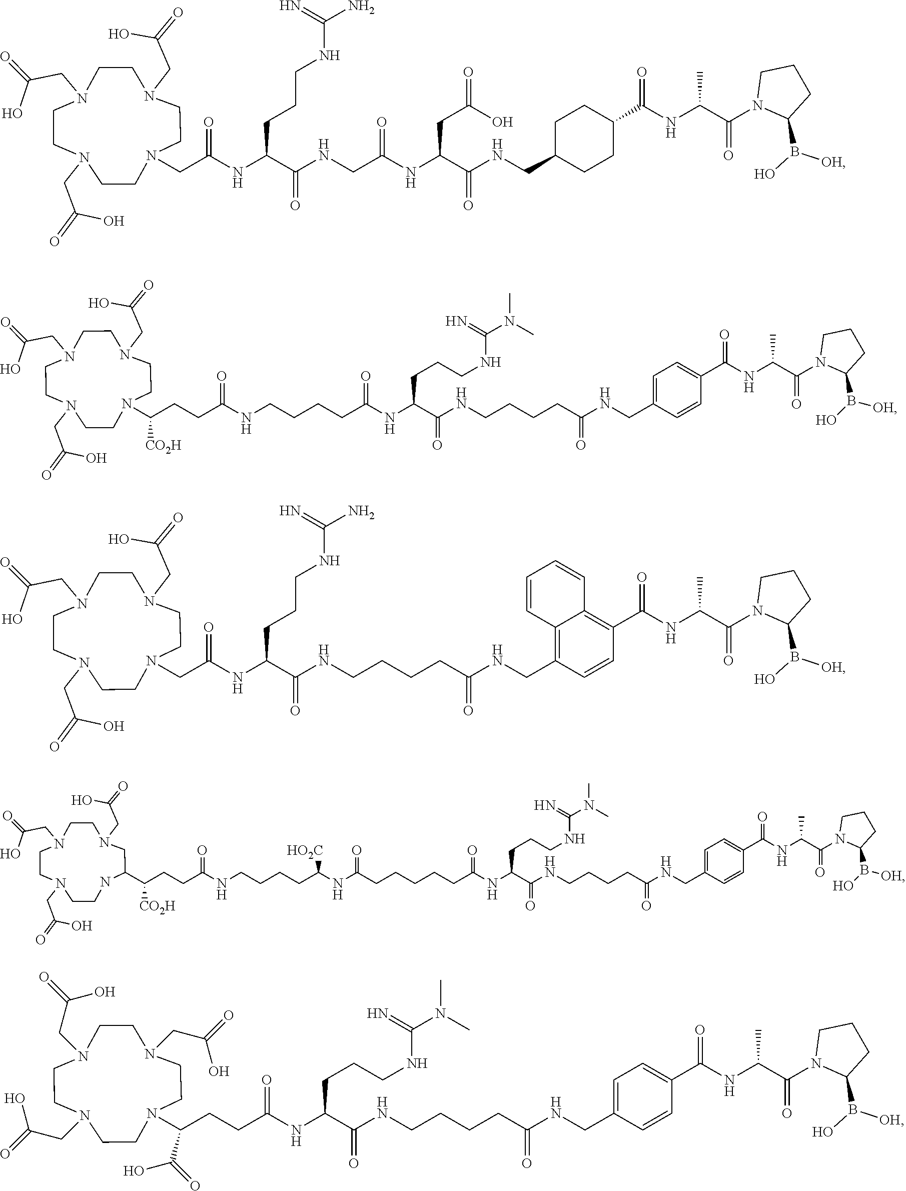

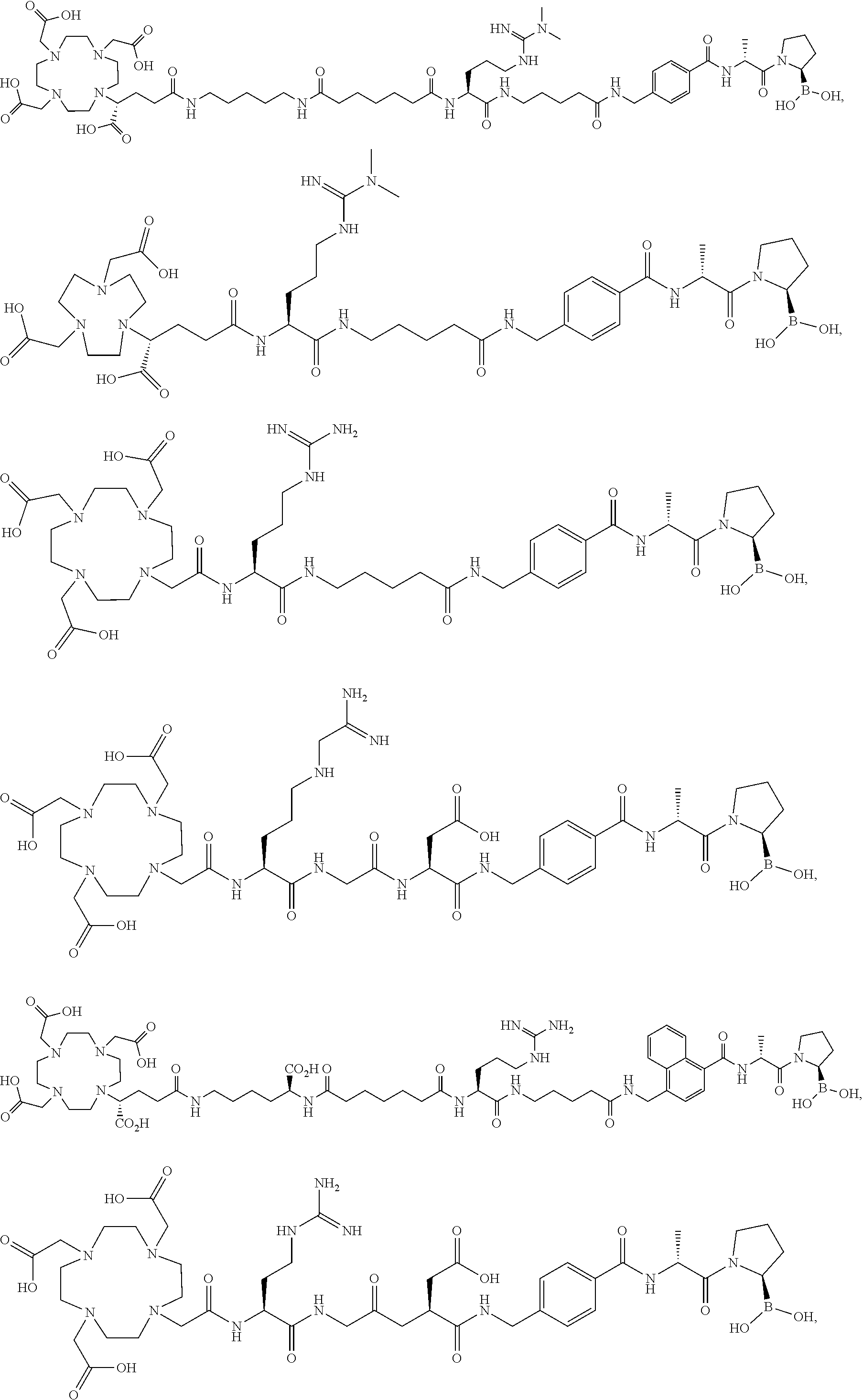

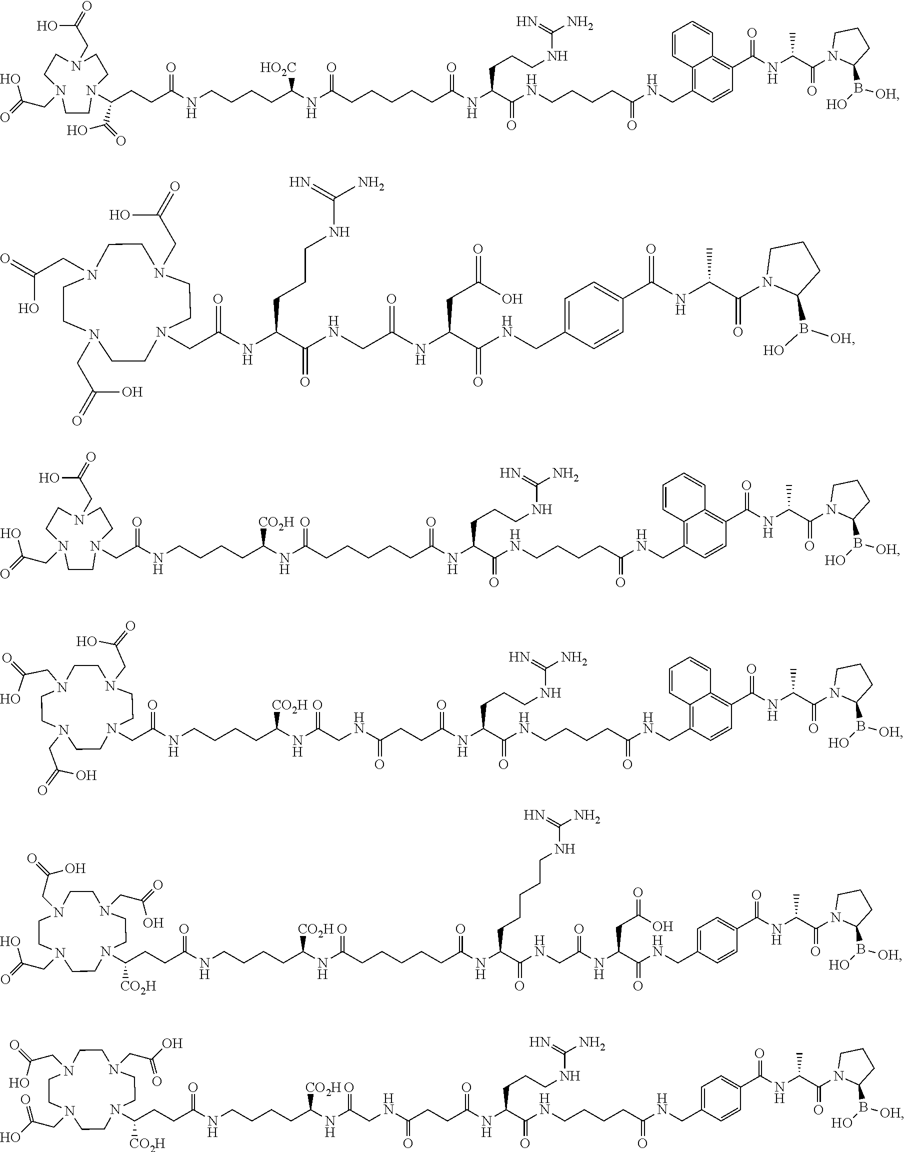

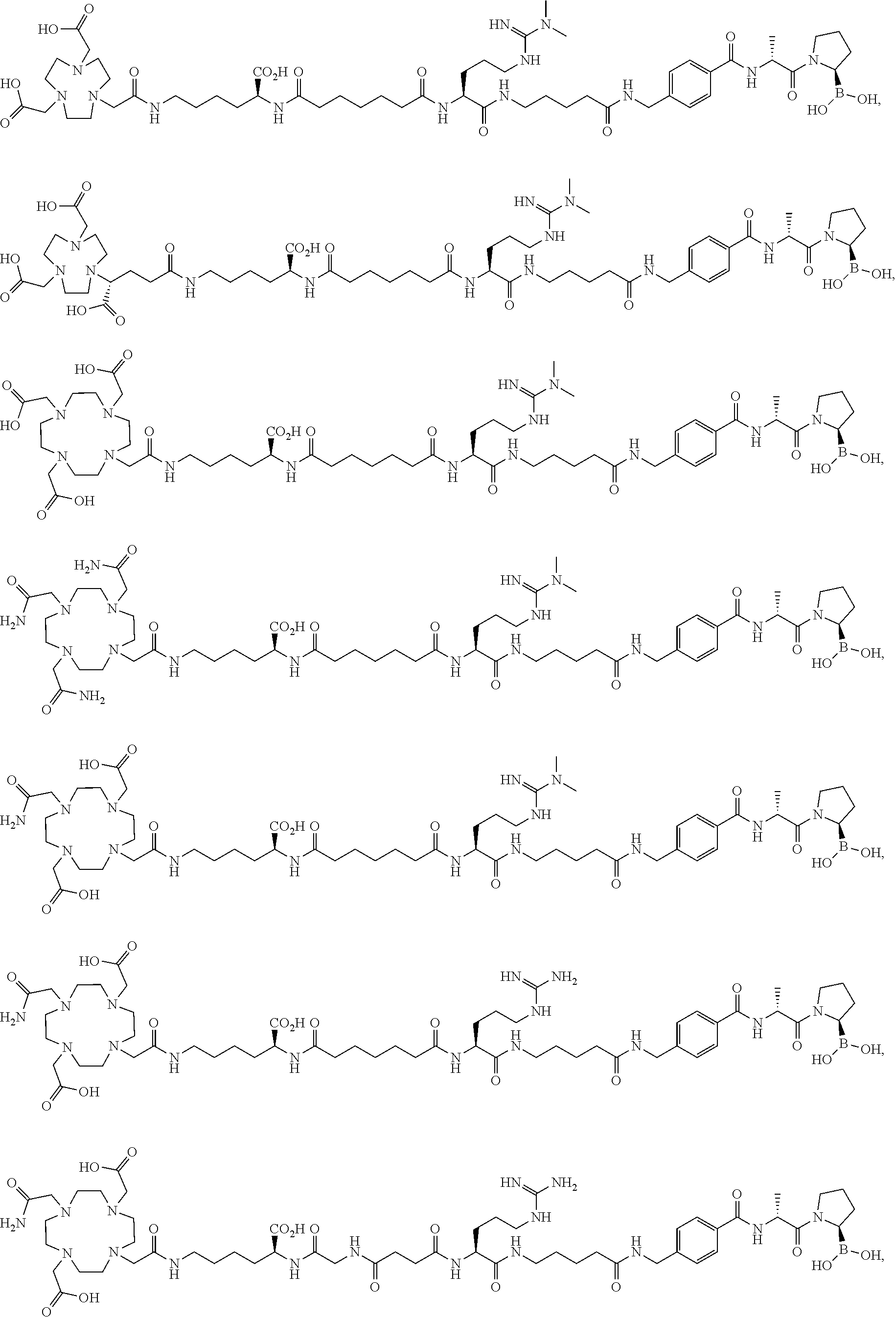

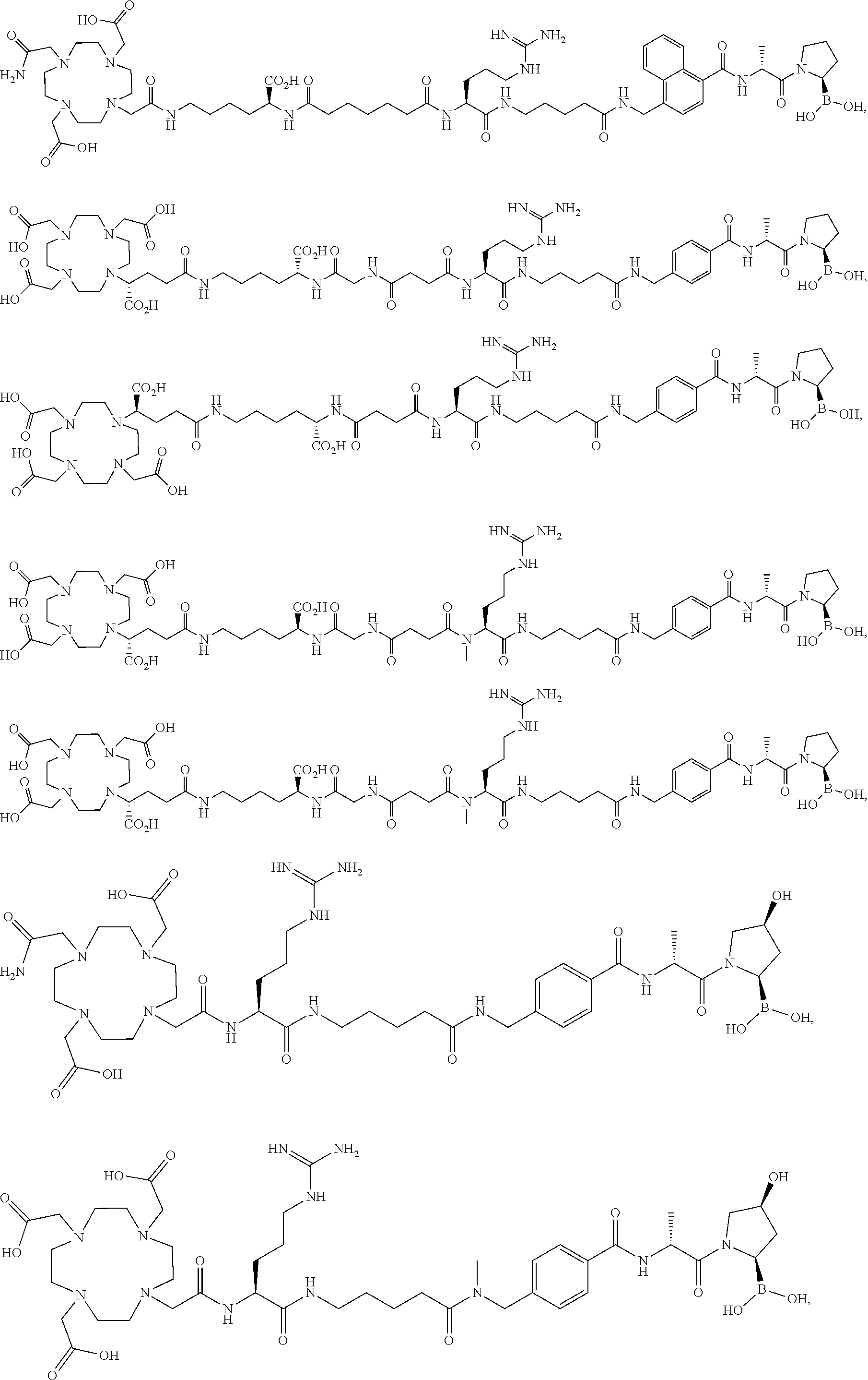

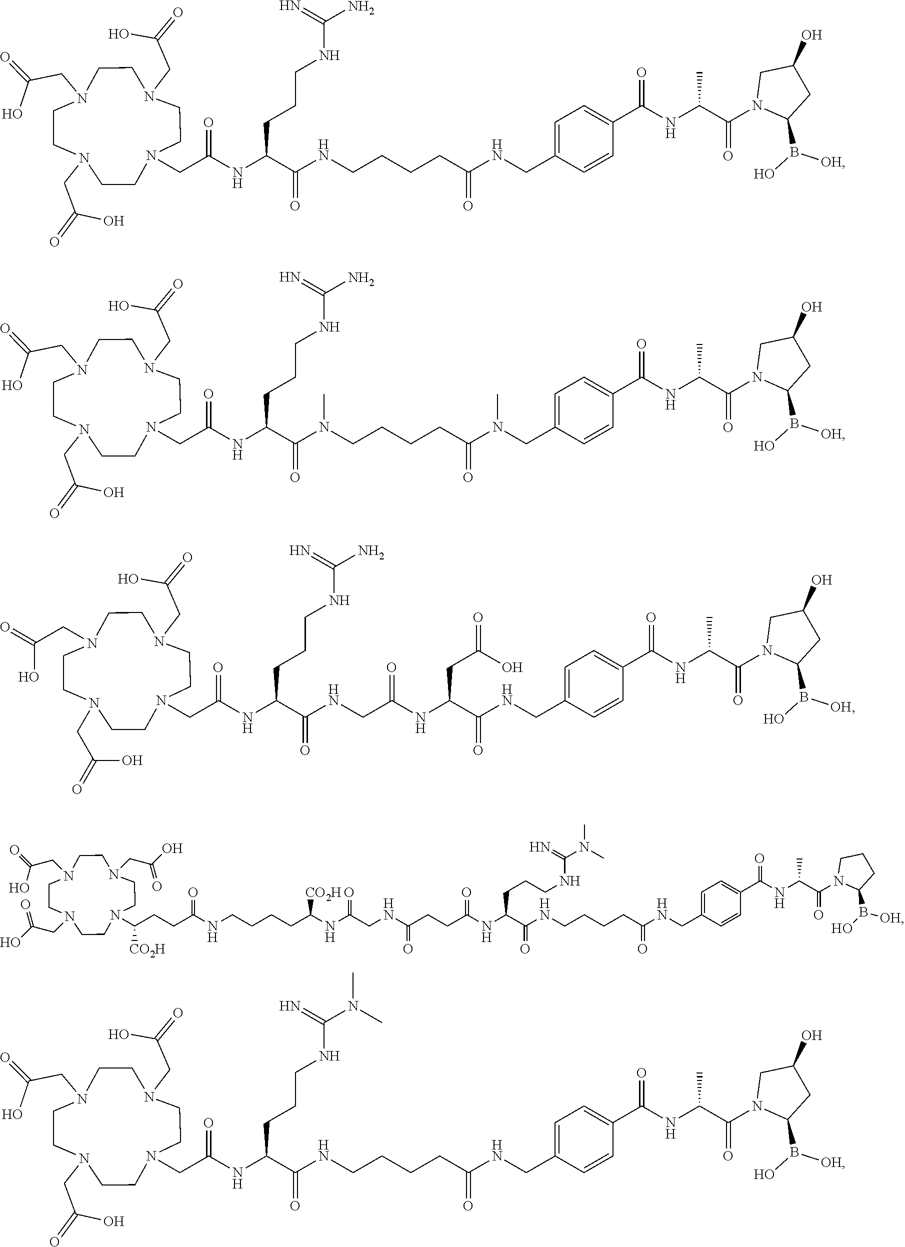

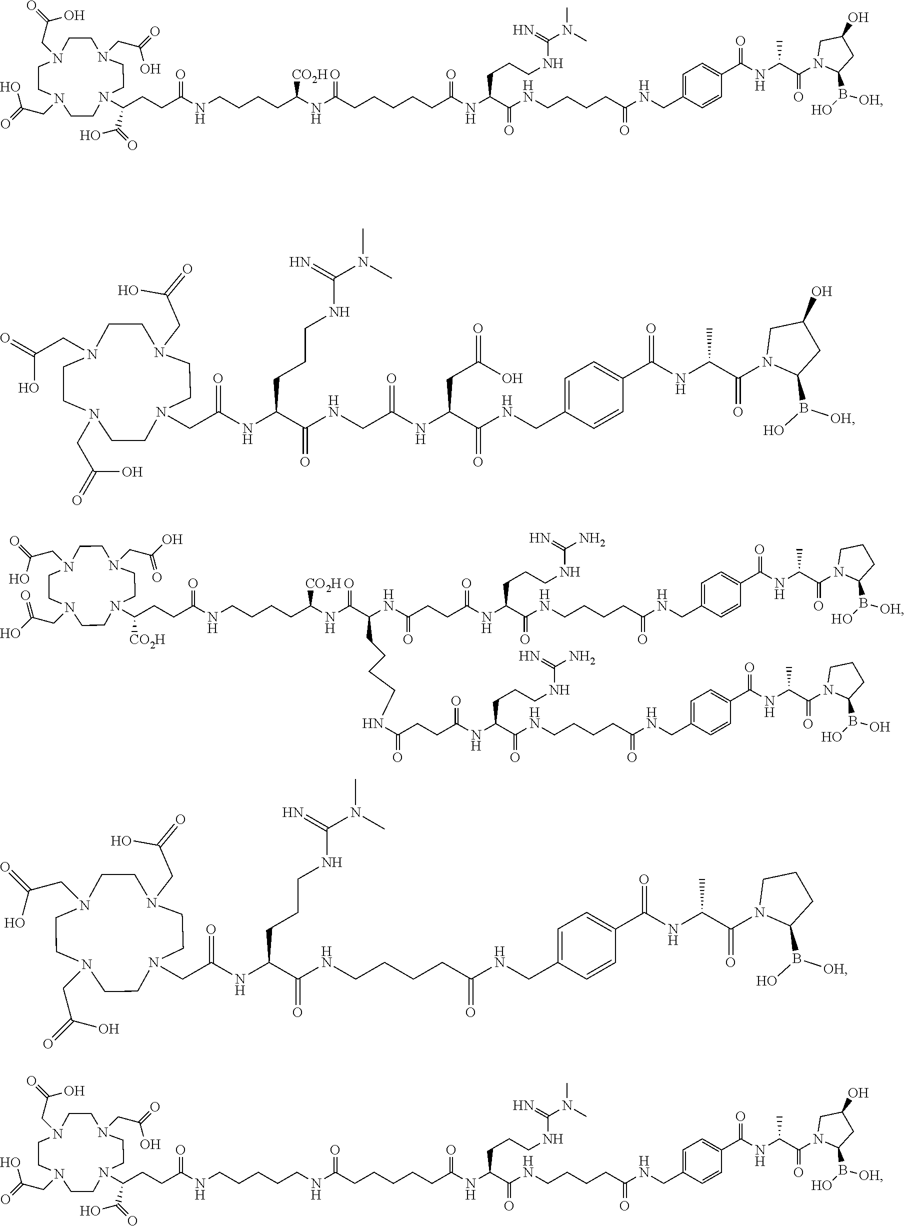

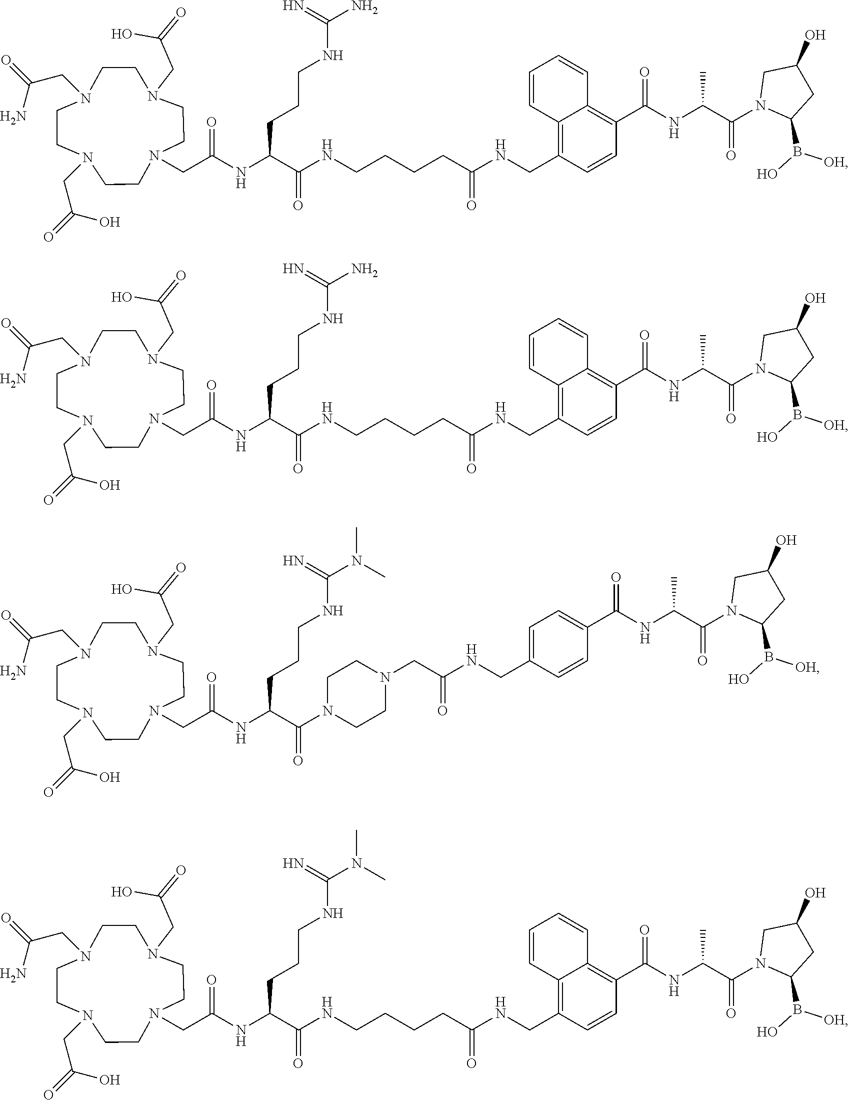

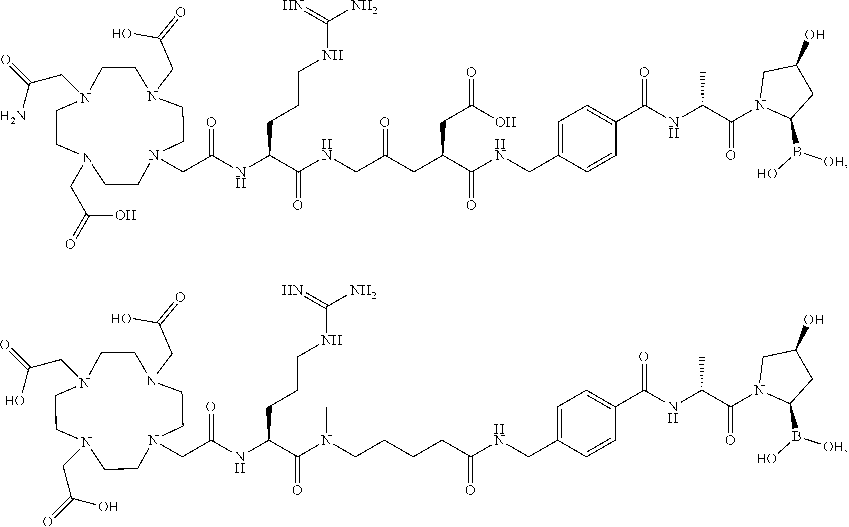 [SMILES: C[C@@H](NC(=O)c1ccc(CNC(=O)[C@H](CC(=O)O)CC(=O)CNC(=O)[C@H](CCCNC(=N)N)NC(=O)CN2CCN(CC(=O)O)CCN(CC(N)=O)CCN(CC(=O)O)CC2)cc1)C(=O)N1C[C@@H](O)C[C@H]1B(O)O ; C[C@@H](NC(=O)c1ccc(CNC(=O)CCCCN(C)C(=O)[C@H](CCCNC(=N)N)NC(=O)CN2CCN(CC(=O)O)CCN(CC(=O)O)CCN(CC(=O)O)CC2)cc1)C(=O)N1C[C@@H](O)C[C@H]1B(O)O]

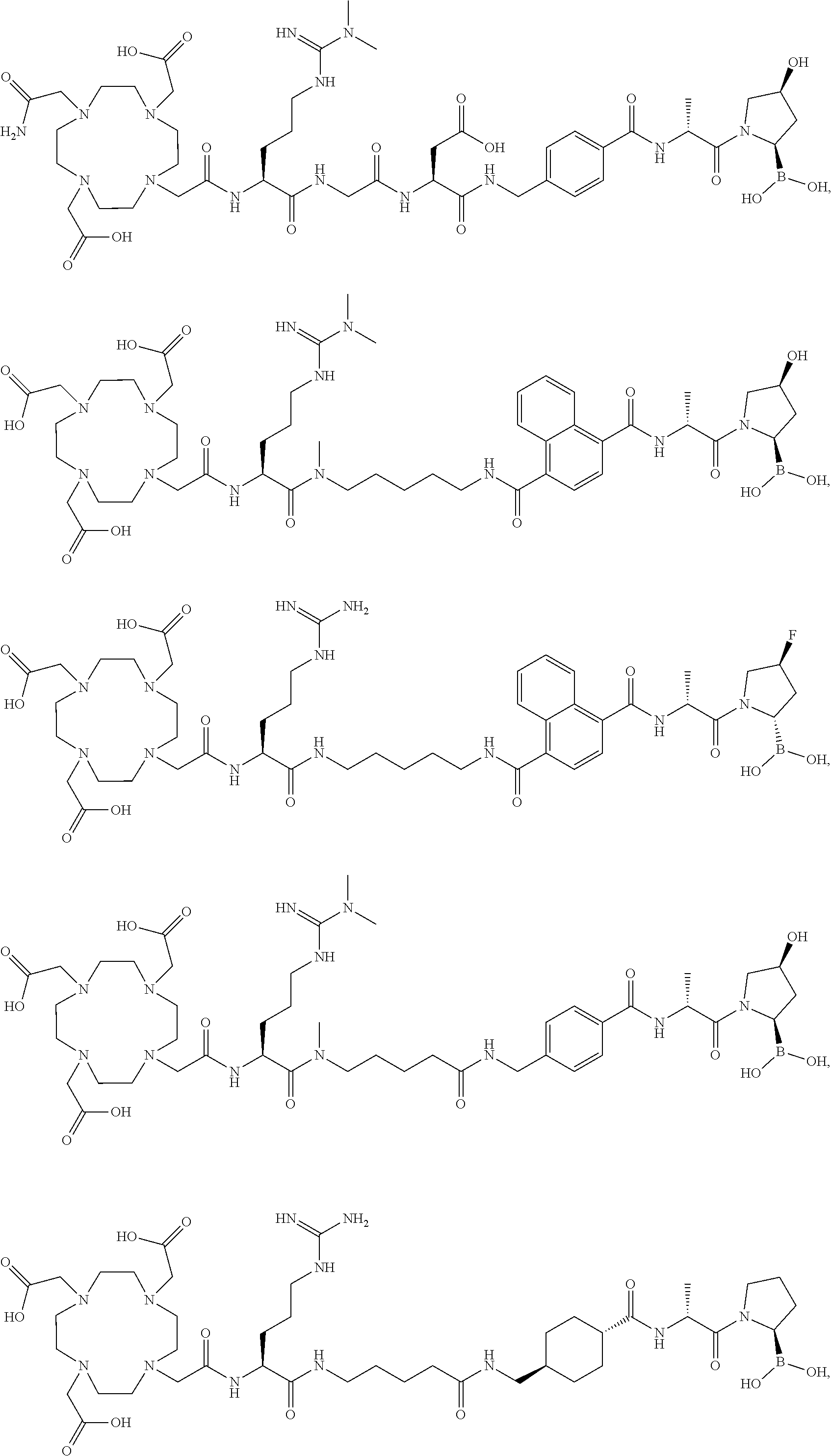

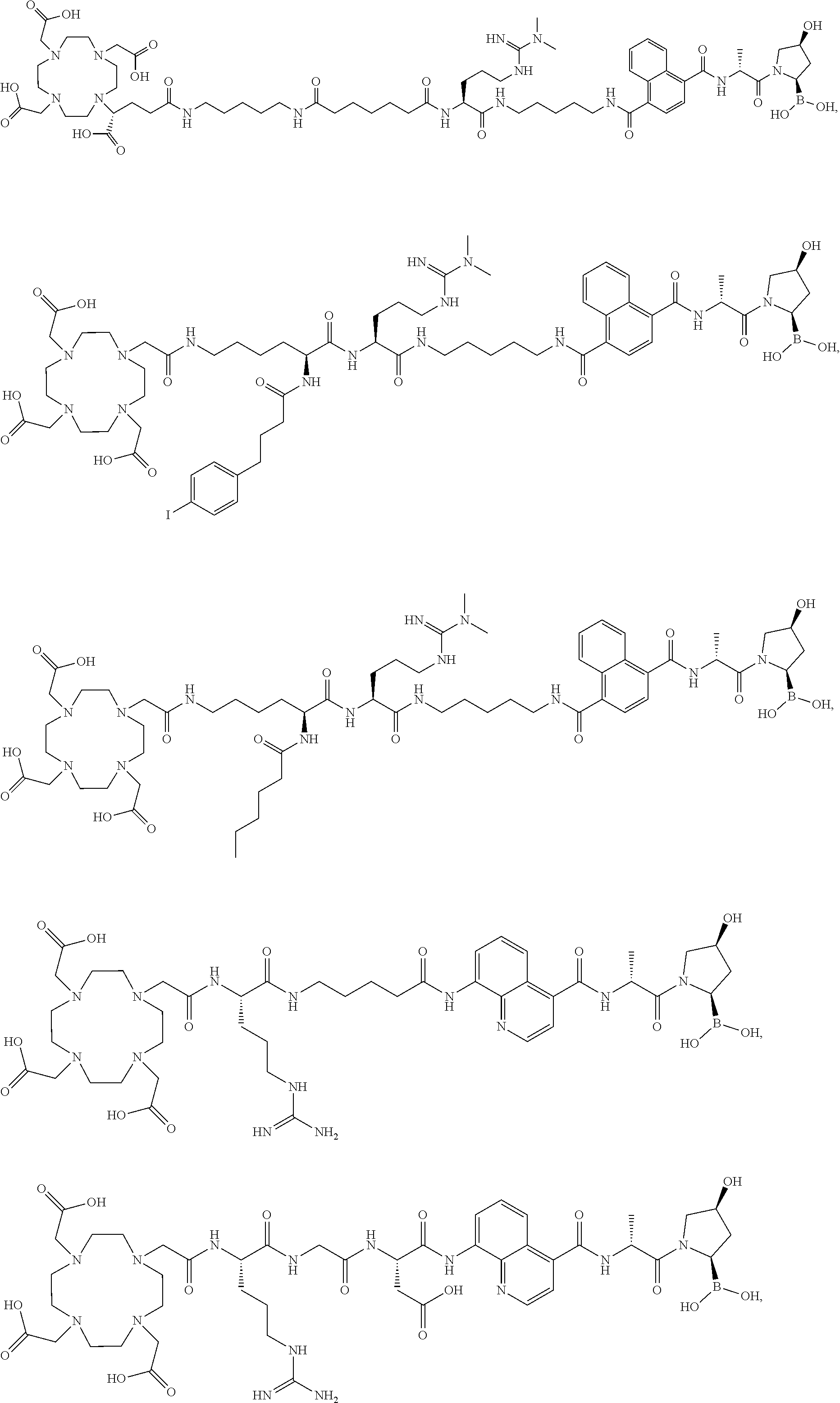

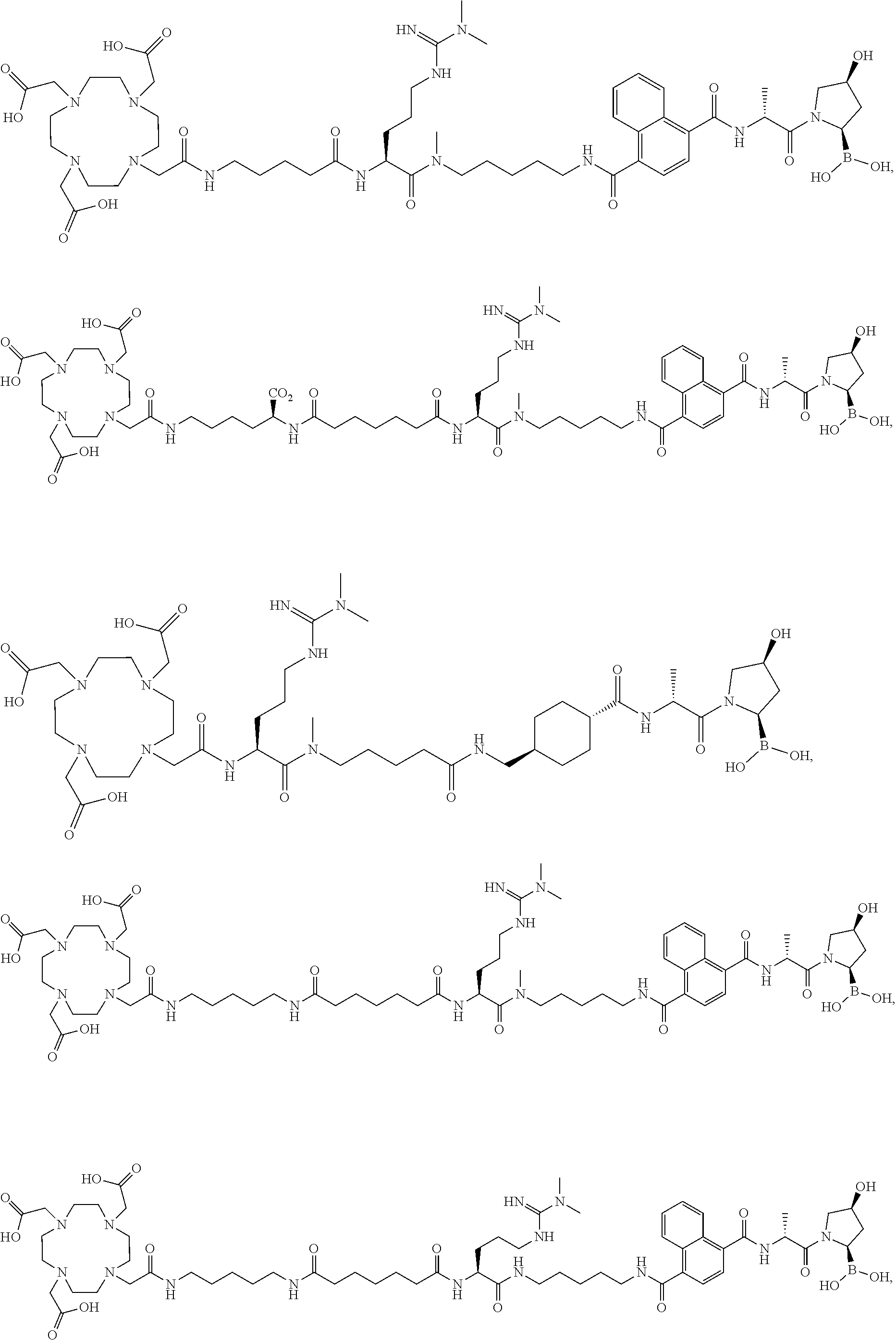

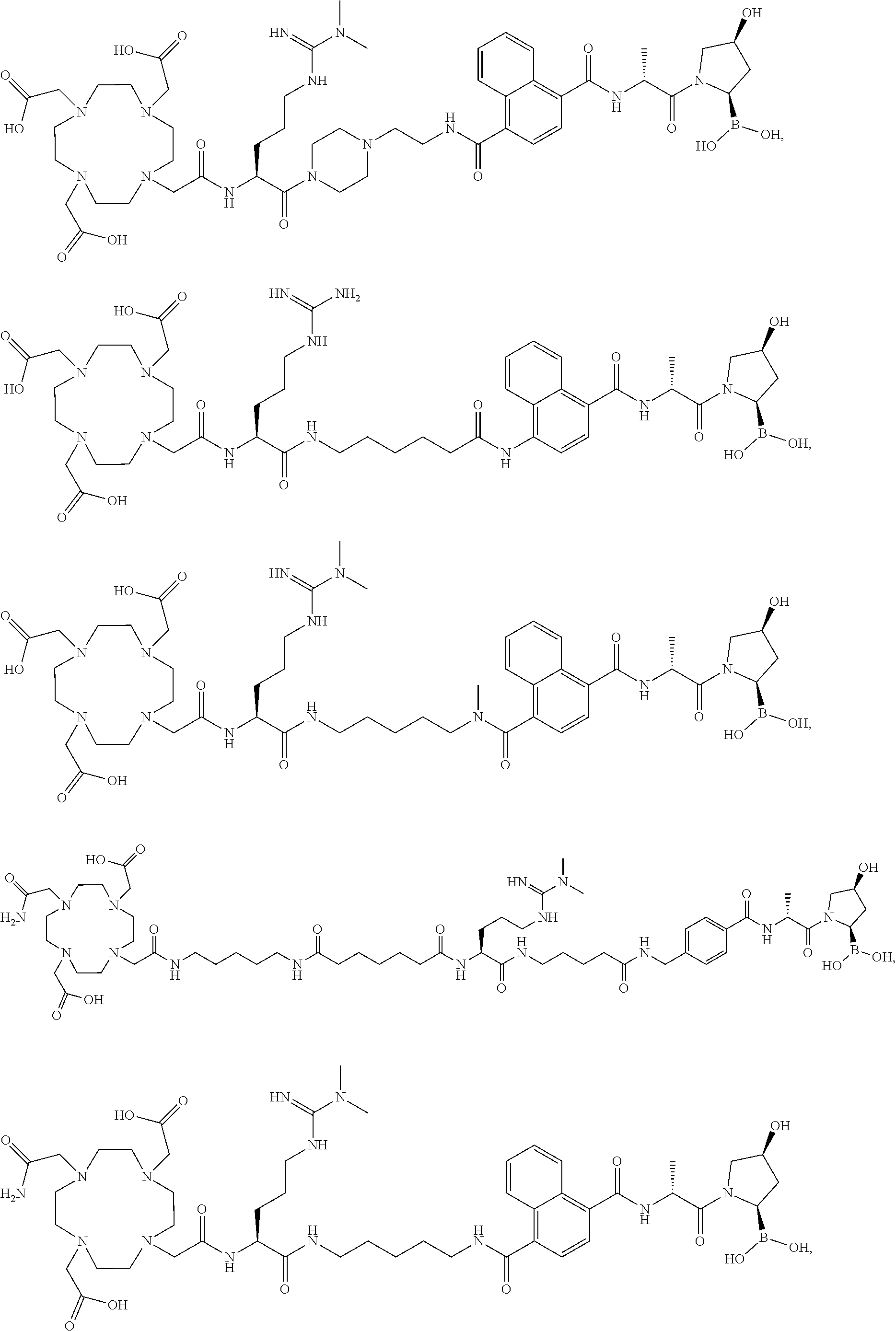

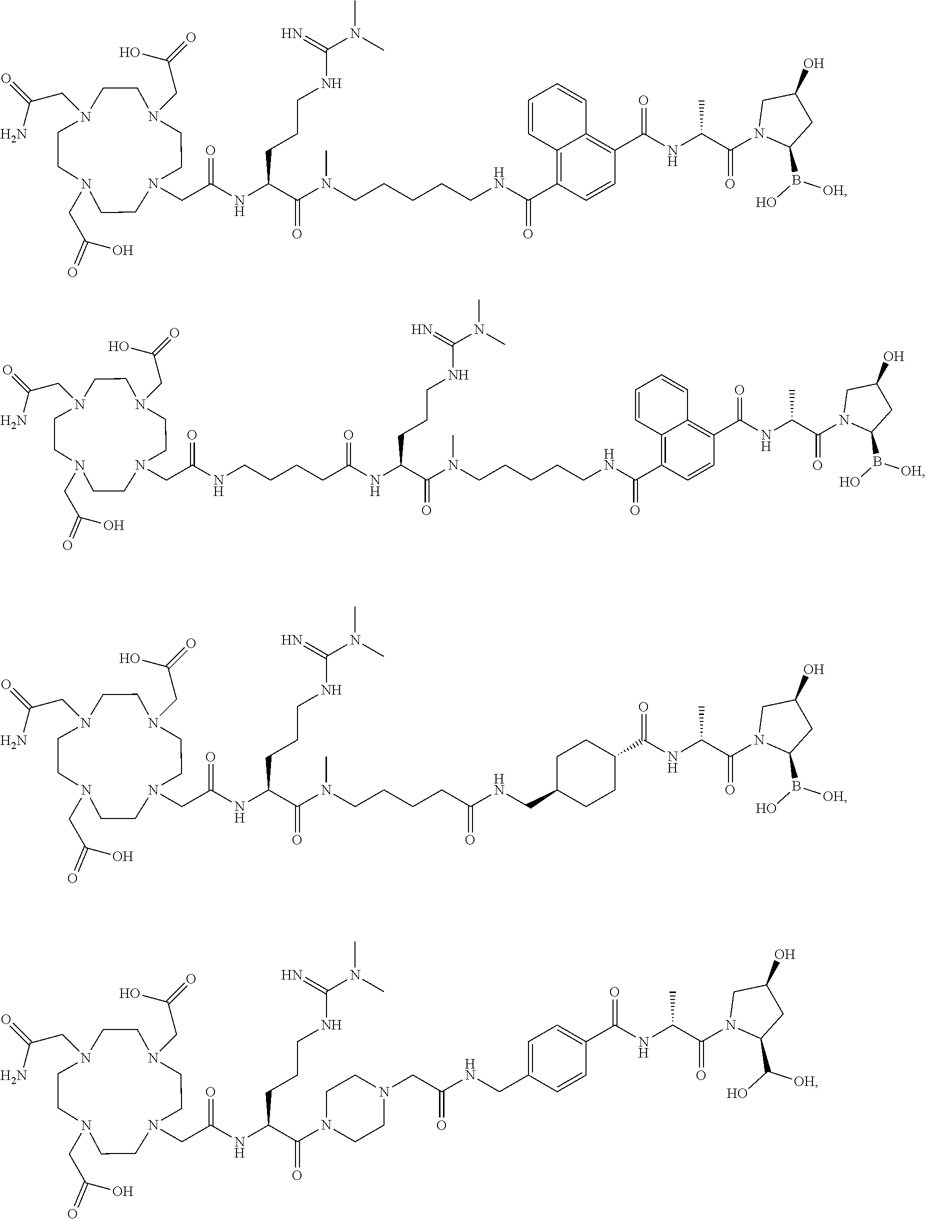

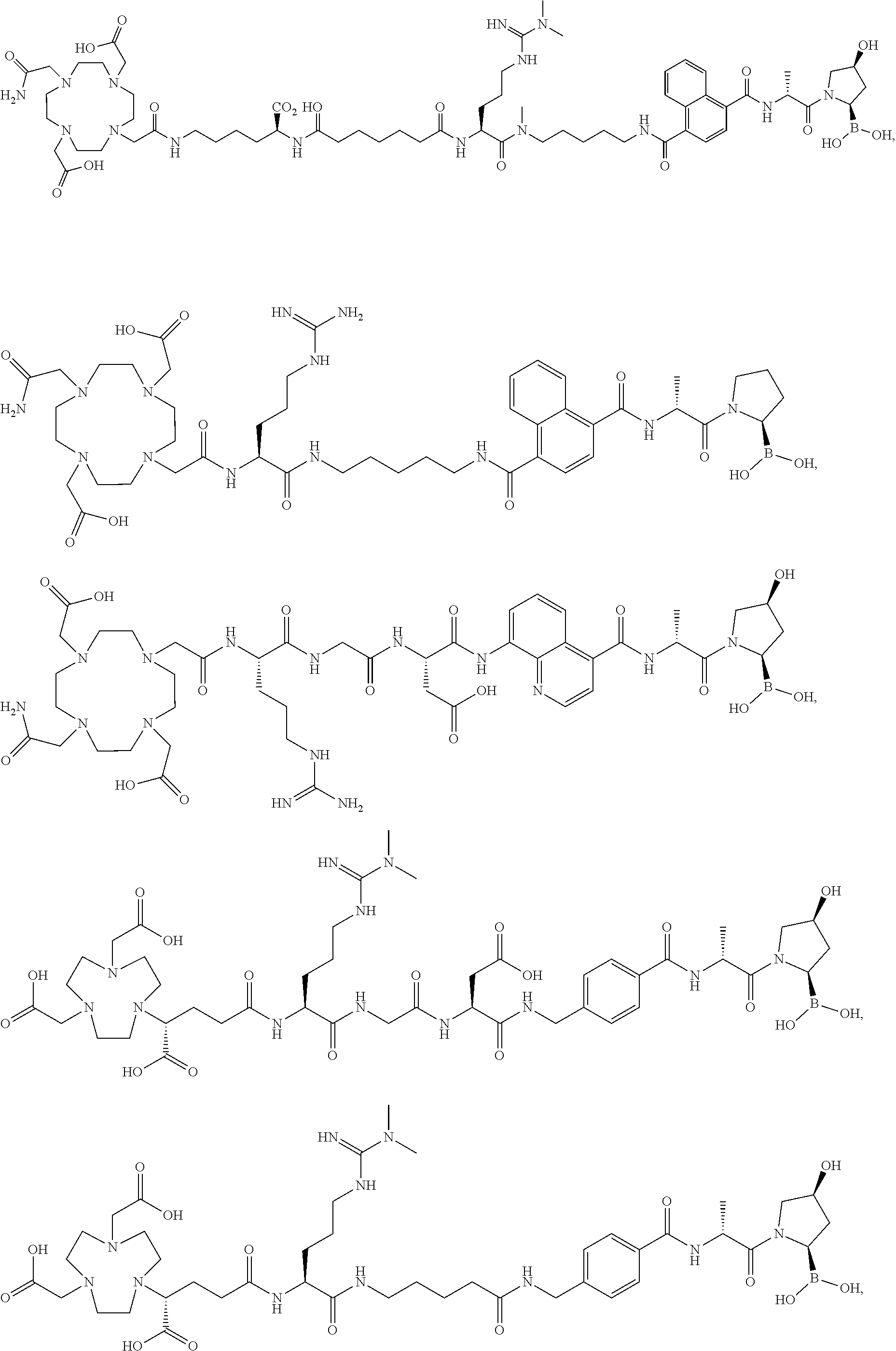

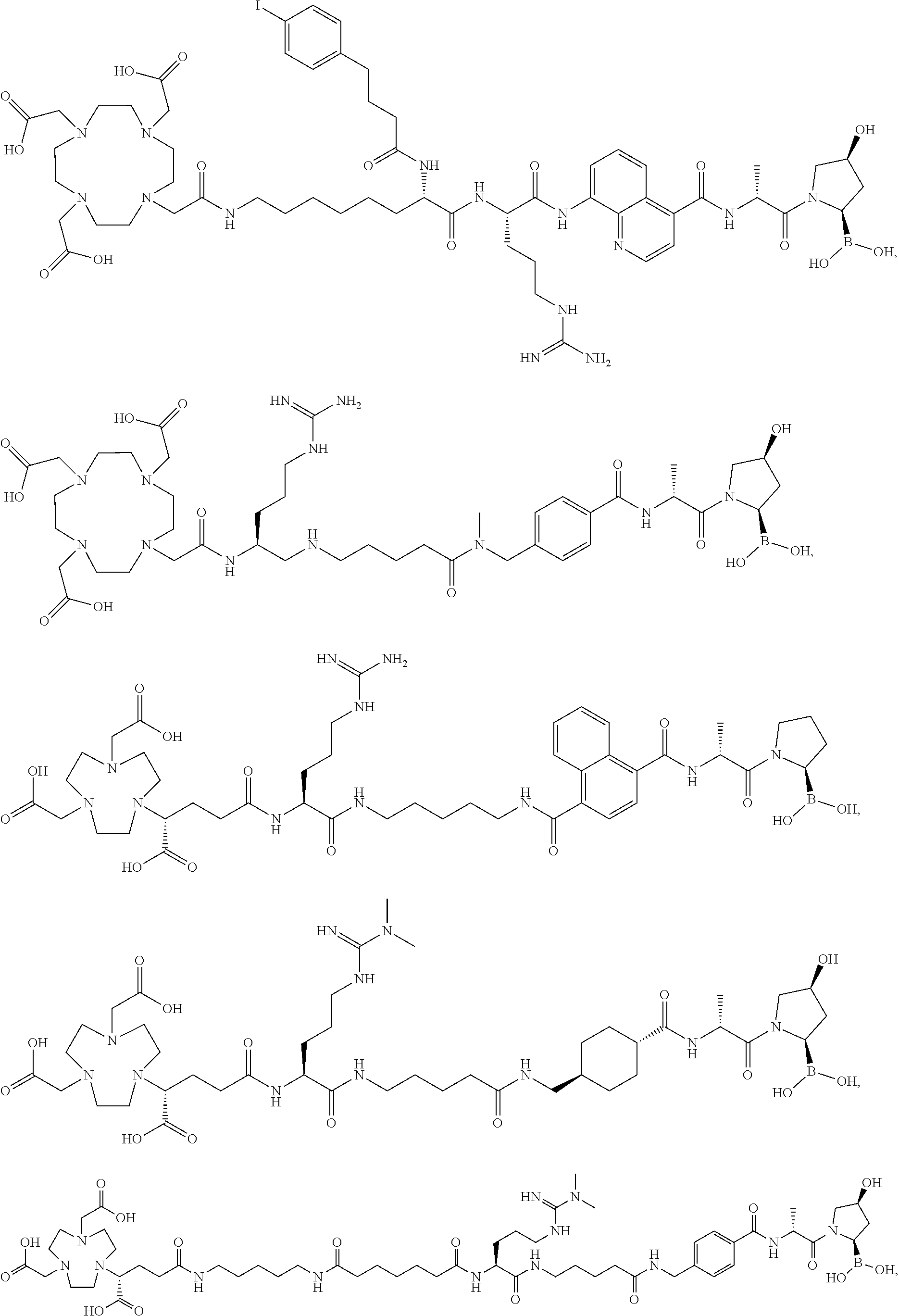

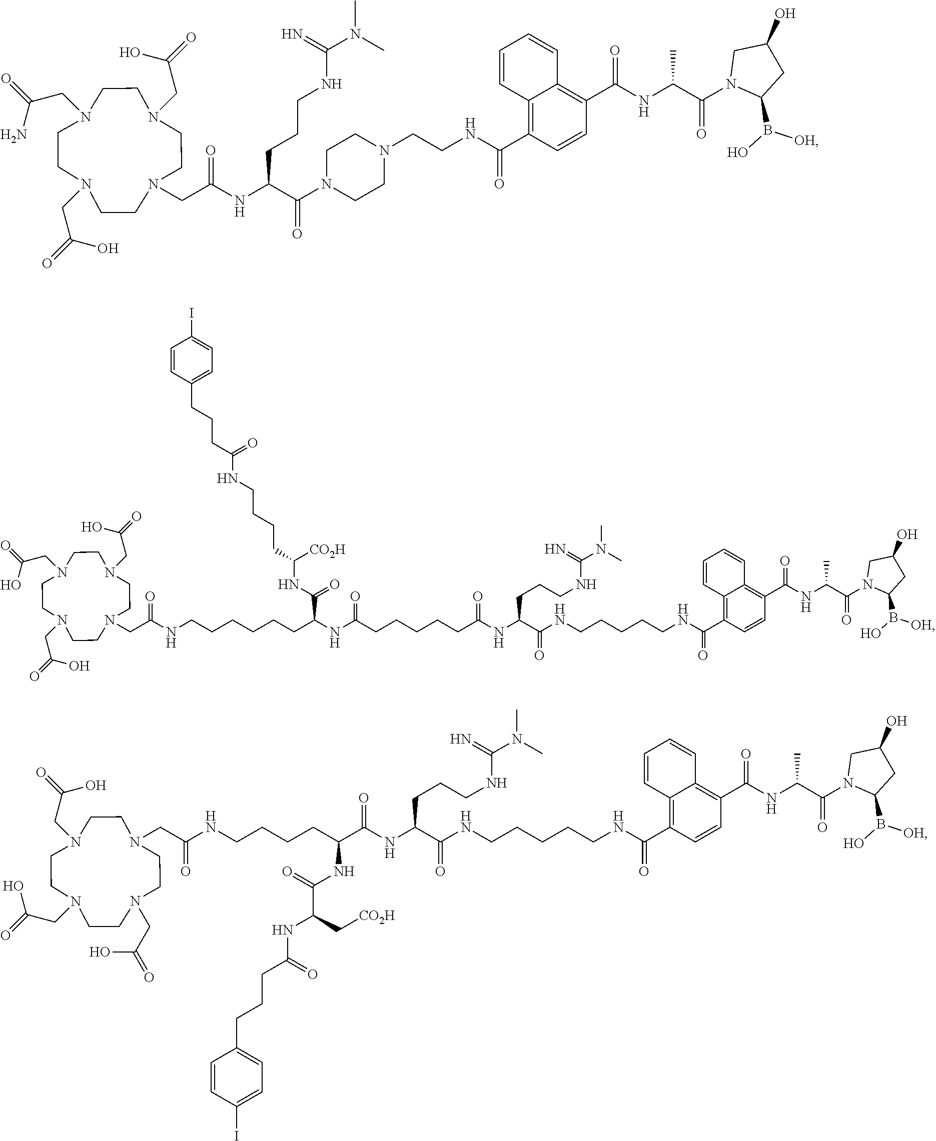

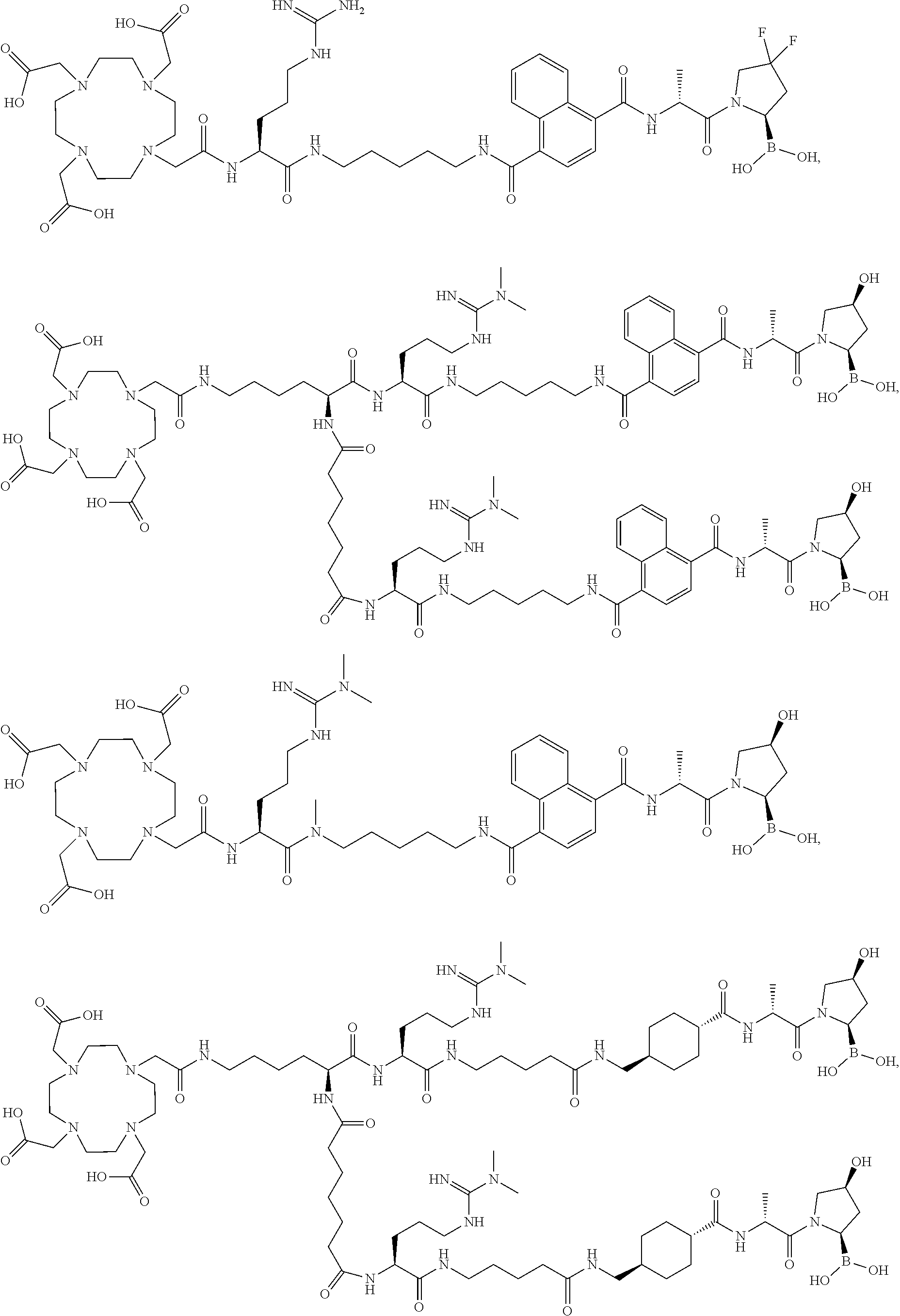

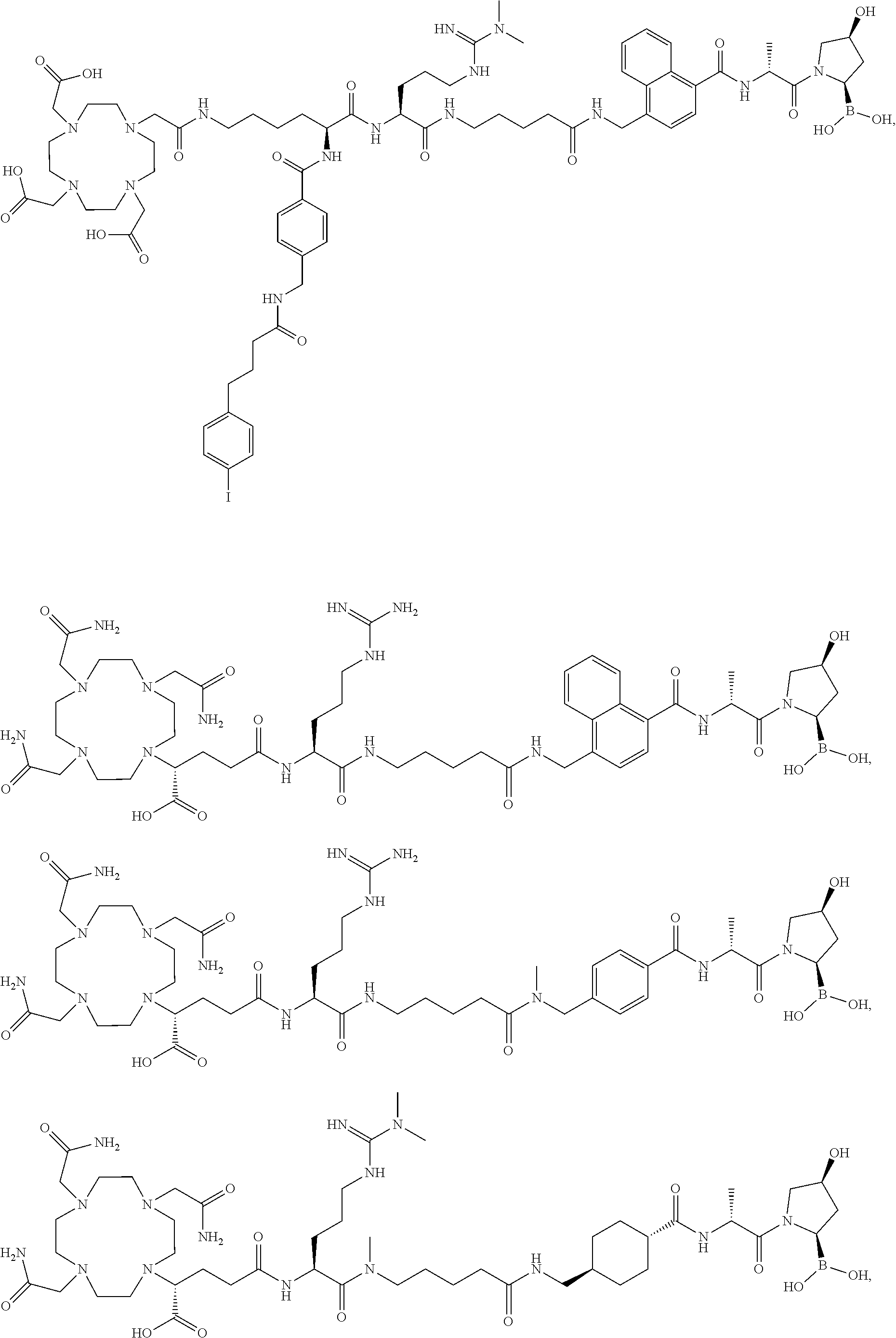

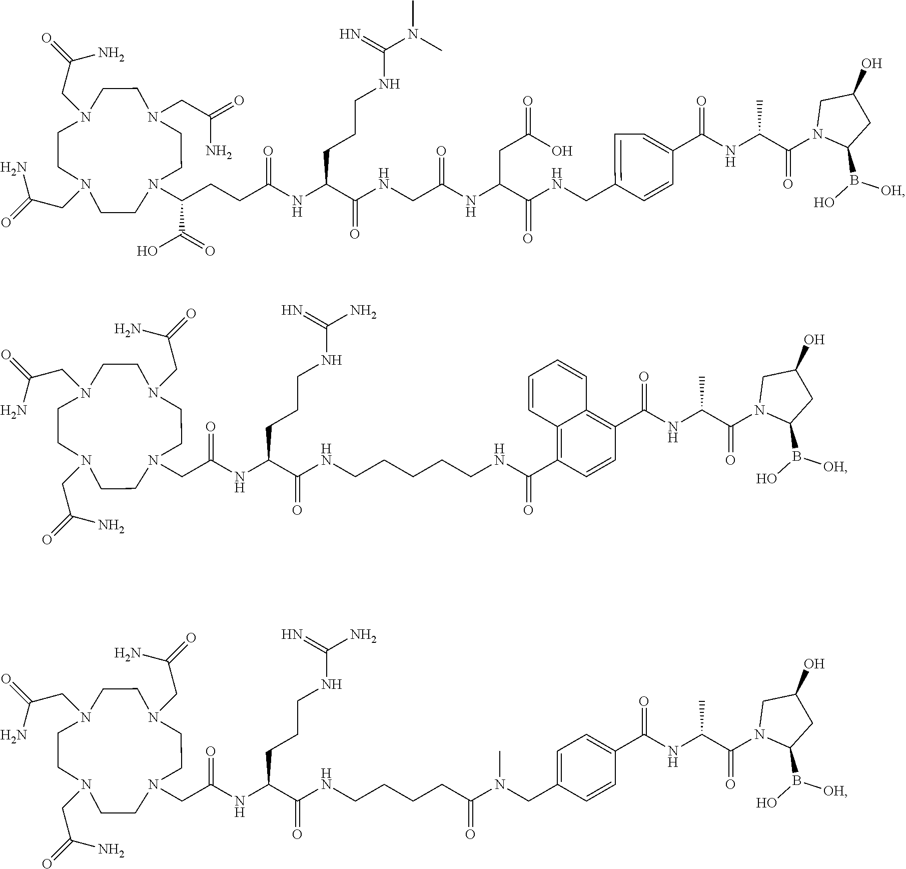 [SMILES: C[C@@H](NC(=O)c1ccc(CNC(=O)C(CC(=O)O)NC(=O)CNC(=O)[C@H](CCCNC(=N)N(C)C)NC(=O)CC[C@H](C(=O)O)N2CCN(CC(N)=O)CCN(CC(N)=O)CCN(CC(N)=O)CC2)cc1)C(=O)N1C[C@@H](O)C[C@H]1B(O)O ; C[C@@H](NC(=O)c1ccc(C(=O)NCCCCCNC(=O)[C@H](CCCNC(=N)N)NC(=O)CN2CCN(CC(N)=O)CCN(CC(N)=O)CCN(CC(N)=O)CC2)c2ccccc12)C(=O)N1C[C@@H](O)C[C@H]1B(O)O ; C[C@@H](NC(=O)c1ccc(CN(C)C(=O)CCCCNC(=O)[C@H](CCCNC(=N)N)NC(=O)CN2CCN(CC(N)=O)CCN(CC(N)=O)CCN(CC(N)=O)CC2)cc1)C(=O)N1C[C@@H](O)C[C@H]1B(O)O]

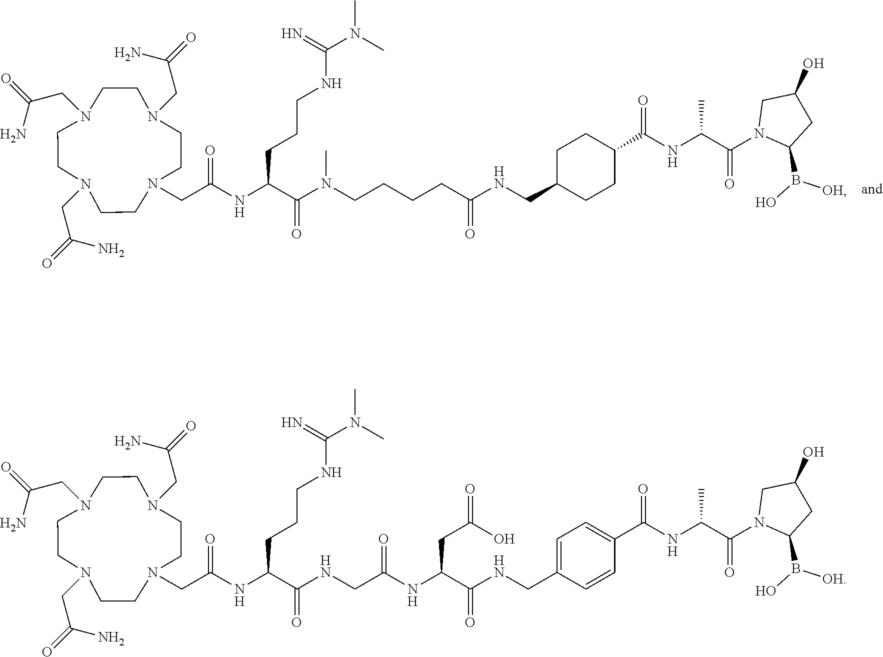 [SMILES: C[C@@H](NC(=O)[C@H]1CC[C@H](CNC(=O)CCCCN(C)C(=O)[C@H](CCCNC(=N)N(C)C)NC(=O)CN2CCN(CC(N)=O)CCN(CC(N)=O)CCN(CC(N)=O)CC2)CC1)C(=O)N1C[C@@H](O)C[C@H]1B(O)O ; C[C@@H](NC(=O)c1ccc(CNC(=O)[C@H](CC(=O)O)NC(=O)CNC(=O)[C@H](CCCNC(=N)N(C)C)NC(=O)CN2CCN(CC(N)=O)CCN(CC(N)=O)CCN(CC(N)=O)CC2)cc1)C(=O)N1C[C@@H](O)C[C@H]1B(O)O]

The chemical drawings in the compounds above contain indications of chiral aspects of the specific compounds shown. However, the chemical drawings in the compounds above do not contain all the possible chiral features of these compounds and the chiral indications shown are not intended to exclude changes to the chiral aspects shown. Thus, alternate chiral versions of the compounds as well as different combinations of chiral attributes are contemplated and included herein.

In an embodiment of a compound of Formula I or a pharmaceutically acceptable salt thereof, wherein X is a chelator bound to a radioactive atom and the radioactive atom is selected from the group consisting of: ²²⁵Ac, ²¹¹At, ⁶⁴Cu, ⁶⁷Cu, ¹⁸F, [¹⁸F]AlF, ⁶⁷Ga, ⁶⁸Ga, ¹¹¹In, ¹⁷⁷Lu, ²⁰³Pb, ²¹²Pb, ¹⁶¹Tb, ⁸⁶Y, ⁹⁰Y, and ⁸⁹Zr.

In an embodiment of a compound of Formula I or a pharmaceutically acceptable salt thereof, wherein the radioactive atom is ²¹²Pb.

In an embodiment of a compound of Formula I or a pharmaceutically acceptable salt thereof, wherein the radioactive atom is ¹⁷⁷Lu.

In an embodiment of a compound of Formula I or a pharmaceutically acceptable salt thereof, wherein the radioactive atom is [¹⁸F]AlF.

In an embodiment of a compound of Formula I or a pharmaceutically acceptable salt thereof, wherein the radioactive atom is chelated with ²²⁵Ac.

Further provided herein are methods of treating cancer, comprising administering to a patient in need thereof, an effective amount of a compound according to Formula I, or a pharmaceutically acceptable salt thereof, or pharmaceutical compositions thereof. In this method, the cancer is characterized by the expression of Fibroblast Activation Protein (FAP) on a tumor, fibroblast, or pericyte within the patient, and wherein the chemical compound selectively binds to Fibroblast Activation Protein and inhibits the proliferation of the Fibroblast Activation Protein-expressing cells. In this method, the cancer can be breast cancer, colorectal cancer, pancreatic cancer, non-small cell lung cancer, small cell lung cancer, prostate cancer, ovarian cancer, melanoma, liver cancer, gastric cancer, esophageal cancer, head and neck cancer, bladder cancer, renal cell carcinoma, glioblastoma, thyroid carcinoma, sarcoma, endometrial cancer, cervical cancer, testicular cancer, mesothelioma, neuroendocrine cancer, bone cancer, adenocarcinoma, non-melanoma skin cancer, leukemia, lymphoma, multiple myeloma, adrenal cancer, cholangiocarcinoma, squamous cell carcinoma, diffuse large B cell lymphoma, squamous cell carcinoma, basal cell carcinoma, transitional cell carcinoma, salivary gland tumors, thymoma, pituitary adenoma, Merkel cell carcinoma, peritoneal carcinoma, nasopharyngeal carcinoma, acute lymphoblastic leukemia, acute myeloid leukemia, chronic lymphocytic leukemia, chronic myeloid leukemia, Hodgkin's lymphoma, non-Hodgkin's lymphoma, uveal melanoma, cutaneous melanoma, mucosal melanoma, gastrointestinal stromal cancer, vulvar cancer, and/or fallopian tube carcinoma. In this method, the cancer can be lung cancer, pancreatic cancer, cervical cancer, esophageal cancer, endometrial cancer, ovarian cancer, cholangiocarcinoma, and/or colorectal cancer. In this method, the cancer can be breast cancer, colorectal cancer, pancreatic cancer, non-small cell lung cancer, small cell lung cancer, ovarian cancer, liver cancer, gastric cancer, esophageal cancer, head and neck cancer, bladder cancer, thyroid carcinoma, sarcoma, cervical cancer, bone cancer, cholangiocarcinoma, salivary gland tumors, gastrointestinal stromal cancer, vulvar cancer, and/or fallopian tube carcinoma. In this method, the cancer can be breast cancer, lung cancer, pancreatic cancer, cervical cancer, esophageal cancer, endometrial cancer, ovarian cancer, cholangiocarcinoma, and/or colorectal cancer.

The cancer may be a subtype of breast cancer or pancreatic cancer that is characterized by particularly high expression of FAP. Such subtypes include hormone receptor-positive (HR+) breast cancer, human epidermal growth factor receptor 2-positive (HER2+) breast cancer, triple-negative breast cancer (TNBC), and pancreatic ductal adenocarcinoma (PDAC). The cancer may also be selected based on confirmation of FAP expression using a molecular assay or imaging modality. As used herein, a “FAP-positive tumor” refers to a tumor confirmed to express Fibroblast Activation Protein at diagnostically or therapeutically relevant levels based on immunohistochemistry (IHC), in situ hybridization (ISH), quantitative PCR, or imaging with a radiolabeled FAP-targeting compound.

In certain embodiments, the methods described herein comprise administering to a patient in need thereof a therapeutically effective amount of a compound according to Formula I, or a pharmaceutically acceptable salt thereof, or a pharmaceutical composition thereof, wherein the administered dose is between 4 megabecquerels (MBq) and 16 MBq. In some embodiments, the administered dose is between 6 MBq and 12 MBq, between 8 MBq and 10 MBq, between 4 MBq and 8 MBq, or between 10 MBq and 16 MBq. In additional embodiments, the administered dose is about 4 MBq, about 6 MBq, about 8 MBq, about 10 MBq, about 12 MBq, about 14 MBq, or about 16 MBq.

The administered dose may be delivered in a single administration or multiple administrations over a defined period, such as once weekly, biweekly, or monthly. The selection of dose and frequency may depend on factors including tumor burden, radiopharmaceutical half-life, patient tolerance, and renal or hepatic function.

The doses described herein may be administered intravenously, intra-arterially, or via direct tumor injection, depending on the indication and radiopharmaceutical properties. In some embodiments, the dose is tailored based on the patient's body weight (e.g., MBq/kg) or body surface area (MBq/m²) to optimize efficacy and minimize toxicity.

In some embodiments, the total administered dose of 4 MBq to 16 MBq is delivered as a single administration or fractionated into multiple administrations over a defined period, such as daily, weekly, biweekly, or monthly.

In certain embodiments, the dose is adjusted based on patient-specific parameters, including, but not limited to, body weight (e.g., MBq/kg), body surface area (MBq/m²), renal clearance, or tumor burden. Also provided herein is a method for diagnosing cancer in a patient, comprising administering to the patient a diagnostically effective amount of a compound according to Formula I, or a pharmaceutically acceptable salt thereof, or pharmaceutical compositions thereof, that selectively binds to Fibroblast Activation Protein, wherein the Fibroblast Activation Protein is expressed on a tumor, fibroblast, or pericyte associated with the cancer; detecting the binding of the compound or salt thereof to the Fibroblast Activation Protein; and correlating the presence of bound compound or salt thereof to the expression of Fibroblast Activation Protein, thereby diagnosing the cancer in the patient.

In embodiments of the diagnostic method, the detection of the compound can be performed using medical imaging techniques, such as positron emission tomography (PET) or single photon emission computed tomography (SPECT), depending on the radionuclide used for labeling. For instance, suitable diagnostic isotopes include ⁶⁸Ga, ¹⁸F, or ^{99m}Tc, which are compatible with PET or SPECT imaging. The resulting images allow visualization of tumors expressing FAP, thereby aiding in diagnosis. Such imaging can be used for tumor localization, for selecting patients for FAP-targeted therapy, and for monitoring response to treatment.

Further provided herein is a method wherein the patient is also administered in simultaneous, separate, or sequential combination an effective amount of one or more of a PD-1 inhibitor, a PD-L1 inhibitor, a CDK4/CDK6 inhibitor, an EGFR inhibitor, an ERK inhibitor, an Aurora A inhibitor, a mitotic inhibitor, a poly-ADP ribose polymerase (PARP) inhibitor, a SHP2 inhibitor, an antimetabolite, a platinum agent, and pemetrexed, or pharmaceutically acceptable salt thereof.

The FAP-targeted compound of Formula I may optionally be administered in combination with one or more additional therapeutic agents to enhance overall efficacy. For example, the radiopharmaceutical can be combined with immune checkpoint inhibitors (such as PD-1 or PD-L1 inhibitors), CDK4/6 inhibitors, PARP inhibitors, EGFR inhibitors, platinum-based chemotherapies, or other conventional chemotherapeutic agents. Administration may occur simultaneously, separately, or sequentially (e.g., the compound of Formula I can be given before or after the co-therapy, or at the same time). Such combinations can produce additive or synergistic anti-tumor effects—for instance, by enhancing immune system activation, by modifying the tumor microenvironment (through stromal targeting), or by sensitizing tumor cells to radiation. This approach recognizes that a multimodal therapy may improve treatment outcomes by attacking the cancer on multiple fronts.

In certain embodiments, the compounds of Formula I act by inhibiting the enzymatic activity of Fibroblast Activation Protein (FAP) on cells within the tumor stroma. By binding to FAP (which is overexpressed on cancer-associated fibroblasts and related stromal cells), the compound can directly block FAP's activity and/or deliver cytotoxic radiation to those FAP-expressing cells. Inhibiting FAP in the tumor microenvironment can disrupt the supportive role of stromal fibroblasts—for example, it may impair tumor-promoting matrix remodeling and reduce the proliferation or viability of FAP-expressing stromal cells. As a result, this mechanism contributes to an overall anti-tumor effect, inhibiting tumor growth and progression. Accordingly, the present disclosure encompasses methods of treating cancer by targeting and inhibiting FAP in a patient through administration of a compound of Formula I.

Further provided herein is a compound according to Formula I, or a pharmaceutically acceptable salt thereof, or pharmaceutical compositions thereof for use in therapy. The compound, or a pharmaceutically acceptable salt thereof, or pharmaceutical compositions thereof can be for use in treating cancer. For this use in treating cancer, the cancer can be breast cancer, colorectal cancer, pancreatic cancer, non-small cell lung cancer, small cell lung cancer, prostate cancer, ovarian cancer, melanoma, liver cancer, gastric cancer, esophageal cancer, head and neck cancer, bladder cancer, renal cell carcinoma, glioblastoma, thyroid carcinoma, sarcoma, endometrial cancer, cervical cancer, testicular cancer, mesothelioma, neuroendocrine cancer, bone cancer, adenocarcinoma, non-melanoma skin cancer, leukemia, lymphoma, multiple myeloma, adrenal cancer, cholangiocarcinoma, squamous cell carcinoma, diffuse large B cell lymphoma, squamous cell carcinoma, basal cell carcinoma, transitional cell carcinoma, salivary gland tumors, thymoma, pituitary adenoma, Merkel cell carcinoma, peritoneal carcinoma, nasopharyngeal carcinoma, acute lymphoblastic leukemia, acute myeloid leukemia, chronic lymphocytic leukemia, chronic myeloid leukemia, Hodgkin's lymphoma, non-Hodgkin's lymphoma, uveal melanoma, cutaneous melanoma, mucosal melanoma, gastrointestinal stromal cancer, vulvar cancer, and/or fallopian tube carcinoma. For this use in treating cancer, the cancer can be lung cancer, pancreatic cancer, cervical cancer, esophageal cancer, endometrial cancer, ovarian cancer, cholangiocarcinoma, and/or colorectal cancer. For this use in treating cancer, the cancer can be breast cancer, colorectal cancer, pancreatic cancer, non-small cell lung cancer, small cell lung cancer, ovarian cancer, liver cancer, gastric cancer, esophageal cancer, head and neck cancer, bladder cancer, thyroid carcinoma, sarcoma, cervical cancer, bone cancer, cholangiocarcinoma, salivary gland tumors, gastrointestinal stromal cancer, vulvar cancer, and/or fallopian tube carcinoma. For this use in treating cancer, the cancer can be breast cancer, lung cancer, pancreatic cancer, cervical cancer, esophageal cancer, endometrial cancer, ovarian cancer, cholangiocarcinoma, and/or colorectal cancer.

Further provided herein is the use in treating cancer wherein the patient is also administered in simultaneous, separate, or sequential combination an effective amount of one or more of a PD-1 inhibitor, a PD-L1 inhibitor, a CDK4/CDK6 inhibitor, an EGFR inhibitor, an ERK inhibitor, an Aurora A inhibitor, a mitotic inhibitor, a poly-ADP ribose polymerase (PARP) inhibitor, a SHP2 inhibitor, an antimetabolite, a platinum agent, and pemetrexed, or pharmaceutically acceptable salt thereof.

The compounds provided herein according to Formula I, or a pharmaceutically acceptable salt thereof, or pharmaceutical compositions thereof, may also be used in the manufacture of a medicament for treating cancer. When used in the manufacture of a medicament, the cancer can be breast cancer, colorectal cancer, pancreatic cancer, non-small cell lung cancer, small cell lung cancer, prostate cancer, ovarian cancer, melanoma, liver cancer, gastric cancer, esophageal cancer, head and neck cancer, bladder cancer, renal cell carcinoma, glioblastoma, thyroid carcinoma, sarcoma, endometrial cancer, cervical cancer, testicular cancer, mesothelioma, neuroendocrine cancer, bone cancer, adenocarcinoma, non-melanoma skin cancer, leukemia, lymphoma, multiple myeloma, adrenal cancer, cholangiocarcinoma, squamous cell carcinoma, diffuse large B cell lymphoma, squamous cell carcinoma, basal cell carcinoma, transitional cell carcinoma, salivary gland tumors, thymoma, pituitary adenoma, Merkel cell carcinoma, peritoneal carcinoma, nasopharyngeal carcinoma, acute lymphoblastic leukemia, acute myeloid leukemia, chronic lymphocytic leukemia, chronic myeloid leukemia, Hodgkin's lymphoma, non-Hodgkin's lymphoma, uveal melanoma, cutaneous melanoma, mucosal melanoma, gastrointestinal stromal cancer, vulvar cancer, and/or fallopian tube carcinoma. When used in the manufacture of a medicament, the cancer can be lung cancer, pancreatic cancer, cervical cancer, esophageal cancer, endometrial cancer, ovarian cancer, cholangiocarcinoma, and/or colorectal cancer. When used in the manufacture of a medicament, the cancer can be breast cancer, colorectal cancer, pancreatic cancer, non-small cell lung cancer, small cell lung cancer, ovarian cancer, liver cancer, gastric cancer, esophageal cancer, head and neck cancer, bladder cancer, thyroid carcinoma, sarcoma, cervical cancer, bone cancer, cholangiocarcinoma, salivary gland tumors, gastrointestinal stromal cancer, vulvar cancer, and/or fallopian tube carcinoma. When used in the manufacture of a medicament, the cancer can be breast cancer, lung cancer, pancreatic cancer, cervical cancer, esophageal cancer, endometrial cancer, ovarian cancer, cholangiocarcinoma, and/or colorectal cancer.

Further provided herein is the use in the manufacture of a medicament wherein the patient is also administered in simultaneous, separate, or sequential combination an effective amount of one or more of a PD-1 inhibitor, a PD-L1 inhibitor, a CDK4/CDK6 inhibitor, an EGFR inhibitor, an ERK inhibitor, an Aurora A inhibitor, a mitotic inhibitor, a poly-ADP ribose polymerase (PARP) inhibitor, a SHP2 inhibitor, an antimetabolite, a platinum agent, and pemetrexed, or pharmaceutically acceptable salt thereof.

The term “pharmaceutically acceptable salt” as used herein refers to a salt of a compound considered to be acceptable for clinical and/or veterinary use. Examples of pharmaceutically acceptable salts and common methodology for preparing them can be found in “Handbook of Pharmaceutical Salts: Properties, Selection and Use” P. Stahl, et al., 2nd Revised Edition, Wiley-VCH, 2011 and S. M. Berge, et al., “Pharmaceutical Salts”, Journal of Pharmaceutical Sciences, 1977, 66(1), 1-19.

Pharmaceutical compositions containing the compounds of Formula I as described herein may be prepared using pharmaceutically acceptable additives. The term “pharmaceutically acceptable additive(s)” as used herein for the pharmaceutical compositions, refers to one or more carriers, diluents, and excipients that are compatible with the other additives of the composition or formulation and not deleterious to the patient. Examples of pharmaceutical compositions and processes for their preparation can be found in “Remington: The Science and Practice of Pharmacy”, Loyd, V., et al. Eds., 22^{nd} Ed., Mack Publishing Co., 2012. Non-limiting examples of pharmaceutically acceptable carriers, diluents, and excipients include the following: saline, water, starch, sugars, mannitol, and silica derivatives; binding agents such as carboxymethyl cellulose, alginates, gelatin, and polyvinyl-pyrrolidone; kaolin and bentonite; and polyethyl glycols.

Further provided herein are kits for the preparation of the radiopharmaceutical compounds. In particular, a kit may comprise an unlabeled (precursor) form of a compound of Formula I in a suitable container, along with instructions for radiolabeling that precursor with a diagnostically or therapeutically effective radionuclide. The kit may further include additional components useful for radiolabeling—for example, one or more buffers, reducing agents, or chelation aids to facilitate incorporation of the radionuclide. Such kits are designed for on-site use in clinical or radiopharmacy settings (e.g., in a hospital radiopharmacy or an automated synthesis module), allowing convenient preparation of the radiolabeled compound immediately prior to use.

As used herein, the term “effective amount” refers to an amount that is a dosage, which is effective in achieve a desired therapeutic result such as treating a disorder or disease, like a cancerous lesion or progression of abnormal cell growth and/or cell division. Factors considered in the determination of an effective amount or dose of a compound include: whether the compound or its salt will be administered; the co-administration of other agents, if used; the species of patient to be treated; the patient's size, age, gender, and general health; the degree of involvement or stage and/or the severity of the disorder; the response of the individual patient; the mode of administration; the bioavailability characteristics of the preparation administered; the dose regimen selected; and the use of other concomitant medication.

A treating physician, veterinarian, or other medical person will be able to determine an effective amount of the compound for treatment of a patient in need. Pharmaceutical compositions can be formulated as a tablet or capsule for oral administration, a solution for oral administration, or an injectable solution. The tablet, capsule, or solution can include a compound of the present invention in an amount effective for treating a patient in need of treatment for cancer.

As used herein, the terms “treating”, “to treat”, or “treatment”, includes slowing, controlling, delaying, reducing, stopping, reversing, preventing, or ameliorating the progression or severity of an existing symptom, disorder, condition, which can include specifically slowing the growth of a cancerous lesion or progression of abnormal cell growth and/or cell division. Treating does not necessarily indicate a total elimination of all disorder or disease symptoms.

As used herein, the term “patient” refers to a mammal in need of treatment. Specifically, the patient can be a human that is in need of treatment for cancer.

Individual isomers, enantiomers, diastereomers, and atropisomers may be separated or resolved at any convenient point in the synthesis of compounds listed below, by methods such as selective crystallization techniques or chiral chromatography (See for example, J. Jacques, et al., “Enantiomers, Racemates, and Resolutions”, John Wiley and Sons, Inc., 1981, and E. L. Eliel and S. H. Wilen, “Stereochemistry of Organic Compounds”, Wiley-Interscience, 1994).

The methods or uses herein can include the steps described herein, and these maybe be, but not necessarily, carried out in the sequence as described. Other sequences, however, also are conceivable. Moreover, individual or multiple steps may be carried out either in parallel and/or overlapping in time and/or individually or in multiply repeated steps. Furthermore, the methods may include additional, unspecified steps.

Compounds of any one of Formula I that are chemically capable of forming salts are readily converted to and may be isolated as a pharmaceutically acceptable salt. Salt formation can occur upon the addition of a pharmaceutically acceptable acid to form the acid addition salt. Salts can also form simultaneously upon deprotection of a nitrogen or oxygen, i.e., removing the protecting group. Examples, reactions and conditions for salt formation can be found in Gould, P. L., “Salt selection for basic drugs,” International Journal of Pharmaceutics, 33: 201-217 (1986); Bastin, R. J., et al. “Salt Selection and Optimization Procedures for Pharmaceutical New Chemical Entities,” Organic Process Research and Development, 4: 427-435 (2000); and Berge, S. M., et al., “Pharmaceutical Salts,” Journal of Pharmaceutical Sciences, 66: 1-19, (1977).

The compounds of the present invention, or salts thereof, may be prepared by a variety of procedures, some of which are illustrated in the Procedures, Preparations and Examples below. The specific synthetic steps for each of the routes described may be combined in different ways, or in conjunction with steps from different routes, to prepare compounds or salts of the present invention. The products of each step in the Preparations below can be recovered by conventional methods, including extraction, evaporation, precipitation, chromatography, filtration, trituration, and crystallization.

### Abbreviations:

ACN Acetonitrile
Bn Benzyl protecting group
Boc t-Butyoxycarbonyl protecting group
tBu tert-Butyl protecting group
2-CTC resin 2-Chlorotrityl chloride resin
DCC N,N′-Dicyclohexylcarbodiimide
DCM Dichloromethane
Dde 2-(4,4-Dimethyl-2,6-dioxocyclohexylidene)ethyl protecting group
DIPEA Diisopropylethylamine
DMF Dimethylformamide
ESI/MS Electrospray ionization mass spectrometry
Et₃N Triethylamine
EtOAc Ethyl acetate
Fmoc 9-Fluorenylmethoxycarbonyl protecting group
h hour(s)
H₂ Hydrogen
HCl Hydrogen chloride
HOBt 1-Hydroxybenzotriazole
K₂CO₃ Potassium carbonate
KHSO₄ Potassium bisulfate
KOH Potassium hydroxide
MeOH Methanol
min minutes
MgSO₄ Magnesium sulfate
mL Milliliters
MTBE Methyl tert-butyl ether
NaCl Sodium chloride
NaHCO₃Sodium bicarbonate
Na₂SO₄ Sodium sulfate
NMP N-Methyl-2-pyrrolidone
P₂O₅ Phosphorus pentoxide
Pbf 2,2,4,6,7-Pentamethyldihydrobenzofuran-5-sulfonyl protecting group
PhB(OH)₂ Phenyl boronic acid
PyBroP Bromo-tris-pyrrolidino-phosphonium hexafluorophosphate
rt Room temperature
SPPS Solid phase peptide synthesis
TBTU 2-(1H-Benzotriazole-1-yl)-1,1,3,3-tetramethylaminium
tetrafluoroborate
TFA Trifluoroacetic acid
THF Tetrahydrofuran
TIPS Triisopropylsilane

### General Procedure for HPLC/MS Analyses:

HPLC/MS was performed on a Thermo LTQXL LC/MS system or Agilent 1290 HPLC/6460 Triple Quad LC/MS system with UV detector (monitoring at 215 nm or 254 nm), using an Agilent 300SB-C8 RP-HPLC column (4.6×100 mm, 3.5 μm) with solvent A (water containing 0.1% TFA) and solvent B (ACN) with a flow rate of 0.5 mL/min at rt. Unless otherwise noted, all HPLC retention times are given for an eluent gradient 2% B for the first 3 min, then from 2% to 98% B over 6 min, which was maintained for the next 6 min.

### General Procedure for Preparative HPLC Purification.

Preparative HPLC separations were run on a Varian semi-preparative system with a reversed phase Discovery C18 569226-U RP-HPLC column (250×21.2 mm; 5 μm particle size, 180 Å). Solvent A (water containing 0.1% TFA) and solvent B (ACN) were mixed in linear binary gradients. The gradients are described as: “10 to 40% B in 30 min”, which means a linear gradient from 10% B (and correspondingly 90% A) to 40% B (and correspondingly 60% A) was run over 30 min. Flow-rate was 20 mL/min. A typical gradient for the purification of the compounds was started at 5-25% B and ended after 20 min at 35-50% B.

### General Procedures for Automated/Semi-Automated Solid-Phase Synthesis.

Automated solid phase synthesis of peptides and polyamides was performed on a PurePep Chorus Peptide Synthesizer on 100 μmol scales. Manual steps were performed in plastic syringes equipped with frits. The amounts of reagents in the protocols described correspond to the 100 μmol scale, unless stated otherwise.

### Synthesis Protocols (SP)

### SP-1: 2-CTC-Resin Loading

2-CTC-resin (Chem-Impex, initial loading 1.43 mmol/g) was swollen in DCM (5 mL) for 30 min and washed with DCM (3 mL, 1 min). The resin was treated with a mixture of naphthalene-1,4-dicarboxylic acid (1.5 eq) in anhydrous DCM with DIPEA (4.5 eq) at rt for 1 h and then drained. The remaining trityl chloride was capped by treating the resin with MeOH (2 mL/g) for 30 min. The resin was thoroughly washed sequentially with DCM (2×), DMF (2×), and MeOH (2×), and stored under vacuum overnight. The loading was determined using the weight difference.

### SP-2: Peptide Synthesis Via HATU Coupling on SPPS

A solution of Fmoc-AA-OH (3.0 eq), HATU (3.0 eq), and DIPEA (6.0 eq) in DMF (10 mL/g resin) was added to the resin-bound free amine compound and reacted for 1 h at rt and then drained. If any coupling reaction was not complete (checked by Kaiser Test), the coupling reaction would be repeated. When complete, the resin was washed with DMF (6×).

### SP-3: On-Resin Fmoc-Deprotection

The resin-bound Fmoc-protected compound was treated with 20% piperidine in DMF (v/v, 10 mL/g) for 15 min and then drained. The resin was again treated with 20% piperidine in DMF (v/v, 10 mL/g) for 15 min and then drained. After deprotection, the resin was washed with DMF (6×).

### SP-4: Cleavage Method from Resin

After the compound had been synthesized, the resin was washed with DCM (5 mL, 4×1 min), treated with 1% TFA in DCM, and agitated for 15 min. This process was repeated two more times. The filtrates were combined, treated with water (4 mL), and the solvent removed under reduced pressure. The residue was purified using the Preparative HPLC purification method written above.

### SP-5: General Procedure for the Coupling Reaction with (2R)-2-amino-1-((2R,4S)-4-hydroxy-2-((3aS,4S,6S)-3a,5,5-trimethylhexahydro-4,6-methanobenzo[d][1,3,2]dioxaborol-2-yl)pyrrolidin-1-yl)propan-1-one HCl or (2R)-2-amino-1-((2R)-2-((3aS,4S,6S)-3a,5,5-trimethylhexahydro-4,6-methanobenzo[d][1,3,2]dioxaborol-2-yl)pyrrolidin-1-yl)propan-1-one

A stirred solution of the protected peptidyl acid (0.1 mmol) in DMF (2.0 mL) was sequentially treated with DIPEA (60 μL, 0.33 mmol), HATU (40 mg, 1.1 mmol), and (2R)-2-amino-1-((2R,4S)-4-hydroxy-2-((3aS,4S,6S)-3a,5,5-trimethylhexahydro-4,6-methanobenzo[d][1,3,2]dioxaborol-2-yl)pyrrolidin-1-yl)propan-1-one HCl (50 mg, 0.17 mmol) at 0° C. The reaction was stirred at rt for 1 h, concentrated under reduced pressure, purified using the Preparative HPLC purification method written above (a typical gradient was 20% B for the first 3 min, then from 20% to 98% B over 12 min, which was maintained for the next 5 min). Both peaks of the desired compound and the de-pinanediol compound were collected, combined, concentrated under reduced pressure, and lyophilized.

### SP-6: General Procedure for the Simultaneous Removal of the —Pbf, -tBu, and Pinanediol Protecting Groups on the Last Step

The protected compound (0.05 mmol) was treated with TFA:H₂O:TIPS (92:4:4, 5 mL). The resulting mixture was stirred at rt until the reaction was complete (typically 2-4 h). The mixture was concentrated under reduced pressure, purified using the Preparative HPLC purification method written above (a typical gradient was 5% B for the first 3 min and then from 5% to 35% B over 17 min) and lyophilized to give the target.

### SP-7: General Procedure for Non-Radioactive Pb Labelling

The precursor (5.4 μmol) was added to a Pb(NO₃)₂ (9.6 mg, 29 μmol) solution in sodium acetate buffer (0.23 M, pH 5.2, 9 mL). The pH of the solution was checked to make sure it was ≥5. The resulting mixture was stirred at 90° C. for 20 min and then was purified using a PLRP-S for Prep to Process column (25×300 mm, 8 μm, 300 Å) with UV detector (monitoring at 215 nm) eluted with a gradient of ACN in water (with 0.05% TFA). The combined fractions were lyophilized to give the non-radioactive Pb-labelled product.

### SP-8: General Procedure for Non-Radioactive Lu Labelling

The precursor (5.4 μmol) was added to a LuCl₃ (8.2 mg, 29 μmol) solution in sodium acetate buffer (0.23 M, pH 5.2, 9 mL). The pH of the solution was checked to make sure it was ≥5. The resulting mixture was stirred at 90° C. for 20 min and then was purified using a PLRP-S for Prep to Process column (25×300 mm, 8 μm, 300 Å) with UV detector (monitoring at 215 nm) eluted with a gradient of ACN in water (with 0.05% TFA). The combined fractions were lyophilized to give the non-radioactive Lu-labelled product.

### Preparations

### Preparation 1

### 5-Benzyl 1-(tert-butyl) (S)-2-hydroxypentanedioate

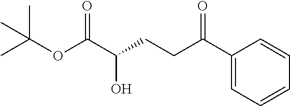 [SMILES: CC(C)(C)OC(=O)[C@@H](O)CCC(=O)c1ccccc1]

A solution of (S)-(+)-5-oxo-2-tetrahydrofurancarboxylic acid (5.0 g, 38.4 mmol) in DCM (100 mL) at 0° C. was treated with DMF (0.2 mL) and oxalyl chloride (8.4 mL, 96 mmol). After stirring a rt for 2 h, the reaction was concentrated under reduced pressure. The residue was concentrated under reduced pressure from DCM (5×100 mL). The residue was dissolved in DCM (10 mL) and added dropwise to a solution of tert-butanol (10 mL) and 2,6-lutidine (10 mL) in DCM (100 mL) at 0° C. The reaction was stirred at rt overnight, diluted with DCM (50 mL), and washed with water (2×25 mL), 5% aqueous citric acid, saturated aqueous NaHCO₃, and saturated aqueous NaCl. The organic layer was collected, dried over Na₂SO₄, filtered, and concentrated under reduced pressure. The residue was purified by silica gel chromatography eluted with 30% to 80% EtOAc in hexane to give (S)-5-(tert-butoxy)-4-hydroxy-5-oxopentanoic acid (6.9 g).

A solution of (S)-5-(tert-butoxy)-4-hydroxy-5-oxopentanoic acid (6.9 g, 28 mmol) in THF (25 mL) was treated with 1M aqueous KOH (30 mL, 30 mmol). The reaction was stirred at 40° C. for 2 h and then concentrated under reduced pressure until dry. The dry solid was further dried over P₂O₅ under reduced pressure for 5 h and suspended in anhydrous DMF (60 mL) and treated with benzyl bromide (4.0 mL). After stirring for 30 h (protected from light), the reaction was poured into ice water (150 mL) and extracted with EtOAc (3×100 mL). The organic layers were combined, washed with saturated aqueous NaCl, collected, dried over Na₂SO₄, filtered, and concentrated under reduced pressure. The residue was purified by silica gel chromatography eluted with 0% to 15% EtOAc in hexane to give the title compound (5.5 g, 49 an % over the synthetic conversions). ESI/MS m/z: 318 (M+H+Na).

### Preparation 95

### tert-Butyl 2-((3aS)-3a,5,5-trimethylhexahydro-4,6-methanobenzo[d][1,3,2]dioxaborol-2-yl)pyrrolidine-1-carboxylate

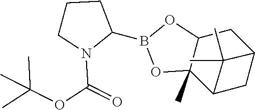

A solution of tert-butyl pyrrolidine-1-carboxylate (20 g, 116.80 mmol) and TMEDA (35 mL, 233.43 mmol) in diethyl ether (200 mL) was cooled to −78° C. The reaction was treated dropwise with sec-BuLi (100 mL, 1.4 M in cyclohexane, 140 mmol) keeping the temperature below −70° C. After addition was complete, allowed the reaction to stir at −78° C. for 4 h. Added triisopropyl borate (48 mL, 350.4 mmol) and allowed the reaction to warm to rt while stirring for 18 h. Quenched the reaction with water (100 mL) and extracted with 2N NaOH (230 mL). The aqueous layer was collected, the pH adjusted to 3 with 2 N HCl, and extracted with EtOAc. The organic layer was collected, dried over MgSO₄, filtered, and concentrated under reduced pressure to give the boronic acid which was treated with (+)-pinanediol (1.1 eq) in diethyl ether and stirred overnight at rt. Concentrated under reduced pressure and purified the residue by silica gel chromatography eluted with 25% EtOAc in hexanes to give the title compound (22.5 g, 55%) as a pale yellow oil.

### Preparation 96

### tert-Butyl (2R,4S)-4-hydroxy-2-((3aS)-3a,5,5-trimethylhexahydro-4,6-methanobenzo[d][1,3,2]dioxaborol-2-yl)pyrrolidine-1-carboxylate

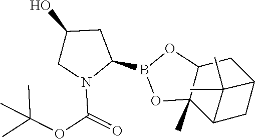

A solution of tert-butyl (S)-3-hydroxypyrrolidine-1-carboxylate (18.9 g, 100 mmol) in anhydrous THF (500 mL) at −78° C. under an Ar atmosphere was treated with TMEDA (34 mL, 221 mmol) and then sec-butyl lithium (158 mL, 1.4 M in cyclohexane, 221 mmol). The resulting bright yellow reaction was warmed to 46° C., stirred for 2 h, and re-cooled to −78° C. When cold, the reaction was treated dropwise with triisopropyl borate (102 mL, 442 mmol). The reaction was allowed to stir and gradually warm to rt over 5 h and then stirred overnight. The reaction was quenched with water (500 mL) and concentrated under reduced pressure to remove most of the THF. Diluted the reaction with diethyl ether (800 mL) and extracted the organic phase with 2M aqueous NaOH (3×250 mL). The pH of the combined aqueous layers was adjusted to pH 3 with 6 N aqueous HCl and extracted with EtOAc (4×1200 mL). The organic layers were combined, dried over MgSO₄, filtered, and concentrated under reduced pressure. The residue was treated with a solution of (+)-pinanediol (170 g, 100 mmol) in diethyl ether (1000 mL) and the reaction stirred at rt for 2 h. Added EtOAc (1000 mL) and washed with water (2×300 mL) and then saturated aqueous NaCl (300 mL). The organic layer was collected, dried over MgSO₄, filtered, concentrated under reduced pressure, and the residue purified by silica gel chromatography eluted with 50% EtOAc in hexane to give the title compound (12.8 g, 44%) as a white powder and the major diastereomer. ESI/MS (m/z): 388 (M+Na), 310 (M-tBu).

### Preparation 97

### tert-Butyl (2R,4R)-4-hydroxy-2-((3aS)-3a,5,5-trimethylhexahydro-4,6-methanobenzo[d][1,3,2]dioxaborol-2-yl)pyrrolidine-1-carboxylate

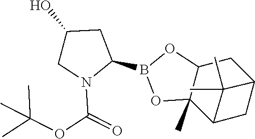

A solution of tert-butyl (2R,4S)-4-hydroxy-2-((3aS)-3a,5,5-trimethylhexahydro-4,6-methanobenzo[d][1,3,2]dioxaborol-2-yl)pyrrolidine-1-carboxylate (11.3 g, 30.8 mmol) in anhydrous THF (400 mL) was cooled to 0° C. under Ar and treated with triphenylphosphine (9.68 g, 37 mmol) and p-nitrobenzoic acid (6.14 g, 36.8 mmol). The reaction was treated dropwise with DIAD (7.4 g, 36 mmol) in anhydrous THF (20 mL) and then stirred for 5 h. The reaction was concentrated under reduced pressure and the residue purified by silica gel chromatography eluted with EtOAc in hexane (1:6). The purified intermediate was dissolved in THF (100 mL) and MeOH (200 mL) and treated with 1M LiOH (120 mL). The reaction was allowed to stir at rt overnight. The reaction was concentrated under reduced pressure and the residue partitioned between EtOAc (500 mL) and water (250 mL). The aqueous layer was extracted with EtOAc (3×250 mL) and the combined organic extracts washed with water (100 mL), saturated aqueous NaCl (100 mL), collected, dried over MgSO₄, filtered, concentrated under reduced pressure, and the residue purified by silica gel chromatography eluted with 50% EtOAc in hexane to give the title compound (7.0 g, 58%) as a white powder. ESI/MS (m/z): 388 (M+Na), 310 (M-tBu).

### Preparation 2

### 5-Benzyl 1-(tert-butyl) (R)-2-(4,7,10-tris(2-(tert-butoxy)-2-oxoethyl)-1,4,7,10-tetraazacyclododecan-1-yl)pentanedioate

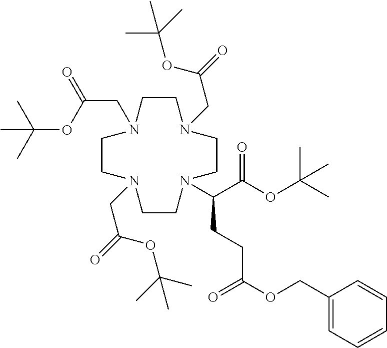 [SMILES: CC(C)(C)OC(=O)CN1CCN(CC(=O)OC(C)(C)C)CCN([C@H](CCC(=O)OCc2ccccc2)C(=O)OC(C)(C)C)CCN(CC(=O)OC(C)(C)C)CC1]

A solution of 5-benzyl 1-(tert-butyl) (S)-2-hydroxypentanedioate (3.46 g, 11.76 mmol) and Et₃N (3.0 mL) in DCM (60 mL) was treated with methanesulfonyl chloride (1.02 mL) at 0-5° C. The reaction was allowed to warm to rt and stir for 1 h. Water (20 mL) was added, and the organic layer removed, washed with saturated aqueous NaCl, collected, dried over Na₂SO₄, filtered, and concentrated under reduced pressure to give the crude mesylate which was used in the next synthetic step without purification.

The mesylate in anhydrous ACN (25 mL) was added dropwise to a mixture of 1,4,7,10-tetraazacyclododecane (6.0 g, 34.8 mmol) and K₂CO₃ (2.0 g, 14.5 mmol) in anhydrous ACN (80 mL) at 50° C. After stirring for 19 h, the reaction was cooled to rt, filtered, and the retained solids washed with ACN (2×20 mL). The filtrate was concentrated under reduced pressure and the residue dissolved in EtOAc (400 mL). This solution was washed with water (80 mL) and saturated aqueous NaCl (30 mL) until the unreacted 1,4,7,10-tetraazacyclododecane had been removed. The organic layer was collected, dried over Na₂SO₄, filtered, and concentrated under reduced pressure to give the acid (5.3 g) which was used in the next synthetic step without purification.

5-Benzyl 1-(tert-butyl) (R)-2-(1,4,7,10-tetraazacyclododecan-1-yl)pentanedioate from the previous synthetic step (5.3 g) and K₂CO₃ (12 g, 87.0 mmol) were suspended in anhydrous ACN (30 mL) and treated dropwise with a solution of tert-butyl bromoacetate (4.8 mL) in anhydrous ACN (6 mL) over 10 min. The resulting reaction was stirred at rt for 3 days, filtered, and the filtrate concentrated under reduced pressure. The residue was dissolved in EtOAc (250 mL) and washed with water (60 mL), saturated aqueous NaHCO₃ (60 mL), and saturated aqueous NaCl. The organic layer was removed, dried over Na₂SO₄, and concentrated under reduced pressure to about 30 mL. The solution was kept at ˜4° C. overnight giving a white precipitate which was collected by filtration, washed with EtOAc (3×8 mL), and dried under high vacuum to give the title compound (6.05 g, 65% over 3 synthetic steps). ESI/MS m/z: 792 (M+H).

### Preparation 3

### (R)-5-(tert-Butoxy)-5-oxo-4-(4,7,10-tris(2-(tert-butoxy)-2-oxoethyl)-1,4,7,10-tetraazacyclododecan-1-yl)pentanoic acid

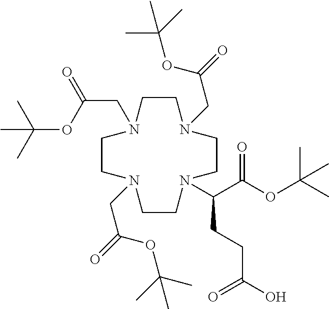 [SMILES: CC(C)(C)OC(=O)CN1CCN(CC(=O)OC(C)(C)C)CCN([C@H](CCC(=O)O)C(=O)OC(C)(C)C)CCN(CC(=O)OC(C)(C)C)CC1]

A solution of 5-benzyl 1-(tert-butyl) (R)-2-(4,7,10-tris(2-(tert-butoxy)-2-oxoethyl)-1,4,7,10-tetraazacyclododecan-1-yl)pentanedioate (6.05 g, 7.65 mmol) in MeOH (50 mL) and water (4 mL) was added to a 100 mL flask containing 10% Pd on C (dry, 1 g) and evacuated and refilled with H₂ three times and then allowed to stir under H₂ at rt for 3 h. The reaction was filtered through diatomaceous earth wetted with MeOH and the filtrate concentrated under reduced pressure (bath temp <35° C.) to a light yellow syrup. The syrup was concentrated from ACN (2×30 mL) to azeotrope residual water and MeOH and then dissolved in ACN (10 mL) and water (20 mL) and lyophilized to give the title compound (5.0 g, 93%) as a white powder. ESI/MS m/z: 700 (M−H).

### Preparation 4

### (R)-2,2′,2″-(10-(1-Carboxy-4-(4-nitrophenoxy)-4-oxobutyl)-1,4,7,10-tetraazacyclododecane-1,4,7-triyl)triacetic acid

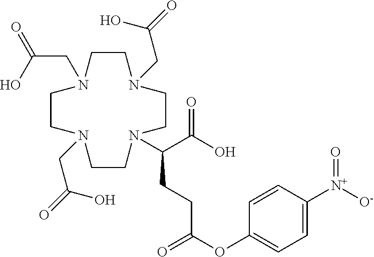 [SMILES: O=C(O)CN1CCN(CC(=O)O)CCN([C@H](CCC(=O)Oc2ccc([N+](=O)[O-])cc2)C(=O)O)CCN(CC(=O)O)CC1]

A solution of (R)-5-(tert-butoxy)-5-oxo-4-(4,7,10-tris(2-(tert-butoxy)-2-oxoethyl)-1,4,7,10-tetraazacyclododecan-1-yl)pentanoic acid (0.21 g, 0.30 mmol) and 4-nitrophenol (0.13 g, 0.90 mmol) in anhydrous ACN (9 mL) was treated with a solution of DCC (0.19 g, 0.90 mmol) in anhydrous pyridine (9 mL) and the reaction stirred at rt for 5 h. The reaction was concentrated under reduced pressure and the residue treated with TFA (15 mL) and triisopropylsilane (1 mL) at 0° C. The reaction was stirred at rt for 20 h and then treated with TFA (5 mL) and stirred at rt until all -tBu groups had been removed (˜40 h). The reaction was concentrated under reduced pressure and the residue concentrated under reduced pressure from DCM (3×30 mL). The residue was suspended in ACN (2 mL) and water (3 mL) and filtered. The retained solids were washed with a mixture of ACN (2 mL) and water (3 mL) and the filtrate purified by using the Preparative HPLC purification method written above to give the title compound as a white solid (102 mg, partial TFA salt, 33% over two synthetic steps). ESI/MS m/z: 598 (M).

### Preparation 98

### (R)-5-(tert-Butoxy)-5-oxo-4-(4,7,10-tris(2-amino-2-oxoethyl)-1,4,7,10-tetraazacyclododecan-1-yl)pentanoic acid

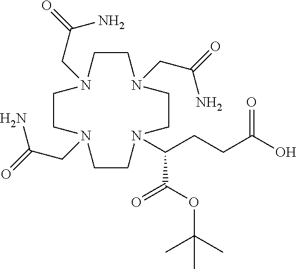 [SMILES: CC(C)(C)OC(=O)[C@@H](CCC(=O)O)N1CCN(CC(N)=O)CCN(CC(N)=O)CCN(CC(N)=O)CC1]

A stirred mixture of 5-benzyl 1-(tert-butyl) (S)-2-hydroxypentanedioate (3.46 g, 11.76 mmol) and Et₃N (3.0 mL) in dry DCM (60 mL) was cooled to 0-5° C. and treated with methanesulfonyl chloride (1.02 mL). After addition was complete, the mixture was allowed to warm to RT and stirred for 1 h. Water (20 mL) was added, the organic phase was separated and washed with brine (20 mL), collected, dried over Na₂SO₄, filtered, and concentrated under reduced pressure to give the mesylate (4.4 g), which was used directly in the next step.

The mesylate was dissolved in dry ACN (25 mL) and added to a mixture of 1,4,7,10-tetraazacyclododecane (6.0 g) and K₂CO₃ (2.0 g) in ACN at 50° C. over 15 min. After stirring at 50° C. for 19 h, the mixture was cooled to rt, filtered, and the solids washed with ACN (2×20 mL). The filtrate was concentrated under reduced pressure, dissolved in EtOAc (400 mL), washed with water (80 mL), saturated aqueous NaCl (4×30 mL). The organic layer was removed, dried over Na₂SO₄, filtered, and concentrated under reduced pressure to give the mono-acylated product (5.3 g) which was used in the next step without purification.

The mono-acyl product (0.45 g) and K₂CO₃ (1.04 g) were suspended in dry ACN (20 mL) and treated with a solution of 2-chloroacetamide (0.37 g, 4 mmol) in dry ACN (5 mL) over 10 min. The resulting mixture was stirred at rt for 1 day, filtered, and concentrated under reduced pressure. The residue was purified by semi-preparative HPLC according to the General Procedure for Preparative HPLC purification. The desired fractions were combined, concentrated under reduced pressure, and co-evaporated with acetonitrile (3×50 ml) in vacuo to give 0.77 g product which was dissolved in MeOH (25 mL) and water (2 mL). The resulting solution was added to a 100 mL flask containing palladium on carbon (dry, 0.4 g). The flask was equipped with a hydrogen balloon and was subjected to three cycles of vacuum and hydrogen purge, then stirred at rt overnight. The resulting mixture was filtered through a methanol-wet bed of diatomaceous earth, and the filtrate was concentrated (bath temperature <35° C. to a light-yellow syrup) under reduced pressure. The syrup was co-evaporated with ACN (2×30 mL) to azeotrope out residual water and to remove any remaining methanol and was then triturated with diethyl ether (20 mL). The upper liquid was discarded, and the solid was dried under vacuum to give the title compound as a pale-yellow powder (0.5 g, 94% yield over 4 steps). ESI/MS (m/z): 530 (M+H).

### Preparation 5

### 7-(4-Nitrophenoxy)-7-oxoheptanoic acid

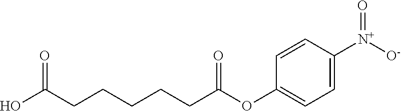 [SMILES: O=C(O)CCCCCC(=O)Oc1ccc([N+](=O)[O-])cc1]

A solution of 7-(tert-butoxy)-7-oxoheptanoic acid (7.8 mmol) and 4-nitrophenol (1.7 g, 12 mmol) in anhydrous ACN (30 mL) was treated with a solution of DCC (2.5 g, 12 mmol) in anhydrous pyridine (20 mL) and the resulting mixture stirred at rt overnight. The reaction was filtered, and the filtrate concentrated under reduced pressure. The residue was purified by silica gel chromatography eluted with 0% to 100% EtOAc in hexane. After purification, 4 mmol of the product was treated with a mixture of DCM (20 mL), TFA (7 mL) and triisopropylsilane (1.5 mL) at 0° C. The reaction mixture was stirred at rt for 1 h. The reaction was concentrated under reduced pressure and the residue was co-evaporated with DCM (3×20 mL). The residue was triturated with diethyl ether to give the title compound as an off-white powder (3.0 g, 90%). ESI/MS m/z: 304 (M+Na).

### Preparation 6

### 4-((2-(4-(tert-Butoxycarbonyl)piperazin-1-yl)ethyl)carbamoyl)-1-naphthoic acid

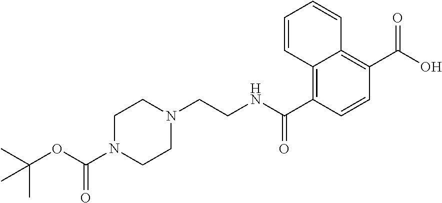 [SMILES: CC(C)(C)OC(=O)N1CCN(CCNC(=O)c2ccc(C(=O)O)c3ccccc23)CC1]

4-(Methoxycarbonyl)naphthalene-1-carboxylic acid was coupled to 1-Boc-4-(2-aminoethyl)-piperazine according to SP-5 and the methyl ester removed by dissolving the ester in cold MeOH (3 mL) and water (3 mL) and treating the solution with 5 M aqueous NaOH (2 mL) and stirring at rt overnight. The reaction was concentrated under reduced pressure, the pH adjusted to 3 with 1M aqueous HCl, and purified using the General Procedure for Preparative HPLC eluting with 20% B to 70% B over 20 min to give the title compound (0.28 g, 52%). ESI/MS m/z: 429 (M+H).

### Preparation 7

### (5S,11S)-11-(2-(tert-Butoxy)-2-oxoethyl)-1-(9H-fluoren-9-yl)-3,6,9-trioxo-5-(3-(3-((2,2,4,6,7-pentamethyl-2,3-dihydrobenzofuran-5-yl)sulfonyl)guanidino)propyl)-2-oxa-4,7,10-triazadodecan-12-oic acid

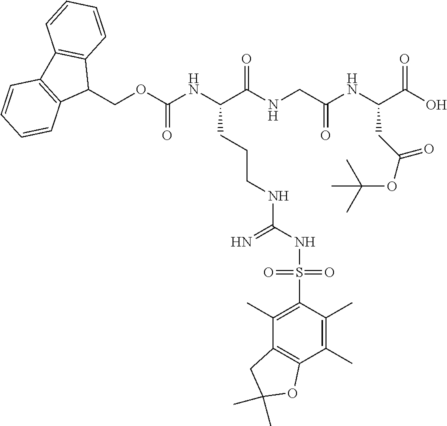 [SMILES: Cc1c(C)c(S(=O)(=O)NC(=N)NCCC[C@H](NC(=O)OCC2c3ccccc3-c3ccccc32)C(=O)NCC(=O)N[C@@H](CC(=O)OC(C)(C)C)C(=O)O)c(C)c2c1OC(C)(C)C2]

The initial resin loading with (S)-2-((((9H-fluoren-9-yl)methoxy)carbonyl)amino)-4-(tert-butoxy)-4-oxobutanoic acid was performed as described in SP-1 on 3 mmol scale. The Fmoc group was removed according to SP-3 and then coupled with (((9H-fluoren-9-yl)methoxy)carbonyl)glycine according to SP-2. The Fmoc group was removed according to SP-3 and then was coupled with N²-(((9H-fluoren-9-yl)methoxy)carbonyl)-N^{w}-((2,2,4,6,7-pentamethyl-2,3-dihydrobenzofuran-5-yl)sulfonyl)-L-arginine according to SP-2. The compound was cleaved from the resin according to SP-4 to give the title compound (2.2 g, 83%). ESI/MS m/z: 877 (M+H).

### Preparation 8

### 8-(5-((((9H-Fluoren-9-yl)methoxy)carbonyl)amino)pentanamido)quinoline-4-carboxylic acid

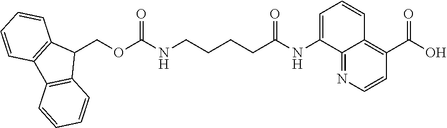 [SMILES: O=C(CCCCNC(=O)OCC1c2ccccc2-c2ccccc21)Nc1cccc2c(C(=O)O)ccnc12]

An ice-cooled solution of 5-((((9H-fluoren-9-yl)methoxy)carbonyl)amino)pentanoic acid (1.6 g, 4.7 mmol) in anhydrous THF (30 mL) was treated with 4-methylmorpholine (0.49 mL, 4.5 mmol) and isobutyl chloroformate (0.57 mL, 4.4 mmol). The reaction mixture was stirred at 0° C. for 1 h and then concentrated under reduced pressure. The residue was dissolved in anhydrous pyridine (15 mL) and treated with 8-aminoquinoline-4-carboxylic acid (282 mg, 1.5 mmol) and DMAP (48 mg, 0.4 mmol). The mixture was stirred at 60° C. overnight and concentrated under reduced pressure. The residue was purified by Preparative HPLC according to the general method to give the title compound (0.16 g, 20%). ESI/MS m/z: 510 (M+H).

### Preparation 9

### 8-((5S,11S)-11-(2-(tert-Butoxy)-2-oxoethyl)-1-(9H-fluoren-9-yl)-3,6,9-trioxo-5-(3-(3-((2,2,4,6,7-pentamethyl-2,3-dihydrobenzofuran-5-yl)sulfonyl)guanidino)propyl)-2-oxa-4,7,10-triazadodecan-12-amido)quinoline-4-carboxylic acid

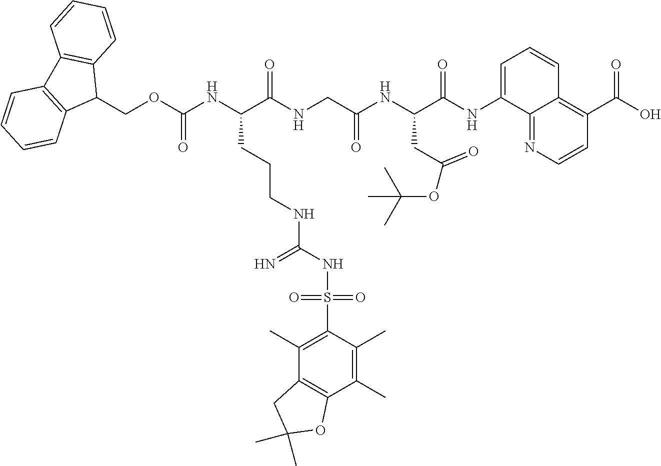 [SMILES: Cc1c(C)c(S(=O)(=O)NC(=N)NCCC[C@H](NC(=O)OCC2c3ccccc3-c3ccccc32)C(=O)NCC(=O)N[C@@H](CC(=O)OC(C)(C)C)C(=O)Nc2cccc3c(C(=O)O)ccnc23)c(C)c2c1OC(C)(C)C2]

An ice-cooled solution of (5S,11S)-11-(2-(tert-butoxy)-2-oxoethyl)-1-(9H-fluoren-9-yl)-3,6,9-trioxo-5-(3-(3-((2,2,4,6,7-pentamethyl-2,3-dihydrobenzofuran-5-yl)sulfonyl)guanidino)propyl)-2-oxa-4,7,10-triazadodecan-12-oic acid (0.7 g, 0.8 mmol) in anhydrous THF (10 mL) was treated with 4-methylmorpholine (0.09 mL, 0.8 mmol) and isobutyl chloroformate (0.10 mL, 0.8 mmol). The reaction mixture was stirred at 0° C. for and concentrated under reduced pressure. The residue was then dissolved in anhydrous pyridine (10 mL). 8-Aminoquinoline-4-carboxylic acid (100 mg, 0.5 mmol) and DMAP (40 mg, 0.33 mmol) were added. The mixture was stirred at 55° C. overnight and then concentrated under reduced pressure. The residue was purified by Preparative HPLC according to the general method to give the title compound (0.11 g, 21%). ESI/MS m/z: 1048 (M+H).

### Preparation 10

### (9H-Fluoren-9-yl)methyl (S,Z)-(1-((5-aminopentyl)(methyl)amino)-5-(((dimethylamino)((2,2,4,6,7-pentamethyl-2,3-dihydrobenzofuran)-5-sulfonamido)methylene)amino)-1-oxopentan-2-yl)carbamate

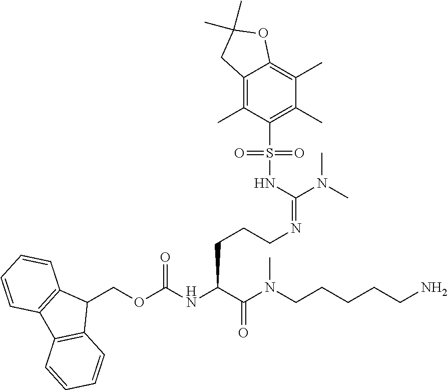 [SMILES: Cc1c(C)c(S(=O)(=O)N/C(=N\CCC[C@H](NC(=O)OCC2c3ccccc3-c3ccccc32)C(=O)N(C)CCCCCN)N(C)C)c(C)c2c1OC(C)(C)C2]

(S,Z)-2-((((9H-Fluoren-9-yl)methoxy)carbonyl)amino)-5-(((dimethylamino)((2,2,4,6,7-pentamethyl-2,3-dihydrobenzofuran)-5-sulfonamido)methylene)amino)pentanoic acid (0.34 g, 0.5 mmol) was coupled to tert-butyl (5-(methylamino)pentyl)carbamate (0.11 g, 0.53 mmol) with HATU (0.2 g, 0.53 mmol) and DIPEA (0.2 mL, 1.15 mmol) in DMF (3 mL) at 0° C. The reaction was stirred at rt for 2 h and then concentrated under reduced pressure. The residue was dissolved in EtOAc (30 mL) and washed with 0.1 M aqueous KHSO₄ (3×5 mL), saturated aqueous NaHCO₃ (3×5 mL), and saturated aqueous NaCl (5 mL). The organic layer was collected, dried over Na₂SO₄, filtered, and concentrated under reduced pressure. The residue was treated with 4M HCl in 1,4-dioxane (4 mL) at 0° C. and stirred at rt for 2 h. The reaction was concentrated under reduced pressure and the residue co-evaporated from DCM (3×10 mL) to give the title compound (0.35 g, 85% over the two steps) as the HCl salt. ESI/MS m/z: 776 (M+H).

### Preparation 11

### (2R)-2-Amino-1-((2R,4S)-4-hydroxy-2-((3aS,4S,6S)-3a,5,5-trimethylhexahydro-4,6-methanobenzo[d][1,3,2]dioxaborol-2-yl)pyrrolidin-1-yl)propan-1-one HCl

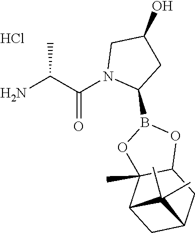

A stirred solution of (tert-butoxycarbonyl)-D-alanine (1.9 g, 10 mmol) in anhydrous DMF (40 mL) was treated sequentially with DIPEA (4.0 mL, 23 mmol), HATU (4.0 g, 10.5 mmol), and (3S,5R)-5-((3aS,4S,6S)-3a,5,5-trimethylhexahydro-4,6-methanobenzo[d][1,3,2]dioxaborol-2-yl)pyrrolidin-3-ol HCl (3.17 g, 10.5 mmol) at 0° C. The reaction was stirred at rt for 2 h and then concentrated under reduced pressure. The residue was dissolved in EtOAc (150 mL) and washed with 0.1 M aqueous KHSO₄ (3×20 mL), aqueous NaHCO₃ (3×20 mL), and saturated aqueous NaCl (30 mL). The organic phase was collected, dried over MgSO₄, filtered, and concentrated under reduced pressure. The residue was purified by silica gel chromatography eluted with 0% to 100% EtOAc in hexane to give the Boc protected compound. ESI/MS m/z: 437 (M+H). The Boc protected compound was treated with 4N HCl in 1,4-dioxane (40 mL) at 0° C. and stirred at rt for 4 h. The reaction was concentrated under reduced pressure and the residue taken up and concentrated under reduced pressure from DCM (40 mL) three times to give the title compound (3.35 g, 90% over two steps) as a white powder. ESI/MS m/z: 337 (M+H).

The following compound in Table 1 was made in a similar way as described for (2R)-2-amino-1-((2R,4S)-4-hydroxy-2-((3aS,4S,6S)-3a,5,5-trimethylhexahydro-4,6-methanobenzo[d][1,3,2]dioxaborol-2-yl)pyrrolidin-1-yl)propan-1-one HCl.

TABLE 1

| | | | ESI/ |
|---|---|---|---|
| Prep | | | MS |
| # | Chemical Name | Structure | m/z |
| 12 | (2R)-2-Amino- 1-((2R)-2- ((3aS,4S,6S)-3a,5,5- trimethylhexahydro-4,6- methanobenzo[d] [1,3,2]dioxaborol- 2-yl)pyrrolidin-1- yl)propan-1-one HCl | 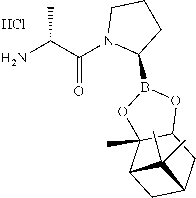 | 321 (M + H) |
| 13 | 2-Amino-1-((2R)- 2-((3aS,4S,6S)- 3a,5,5- trimethylhexahydro-4,6- methanobenzo[d] [1,3,2]dioxaborol- 2-yl)pyrrolidin-1- yl)ethan-1-one HCl | 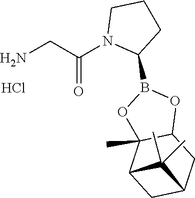 | |

### Preparation 14

### (S)-5-(2-(4-(Benzyloxy)-4-oxobutanamido)-5-(3-((2,2,4,5,7-pentamethyl-2,3-dihydrobenzofuran-6-yl)sulfonyl)guanidino)pentanamido)pentanoic acid

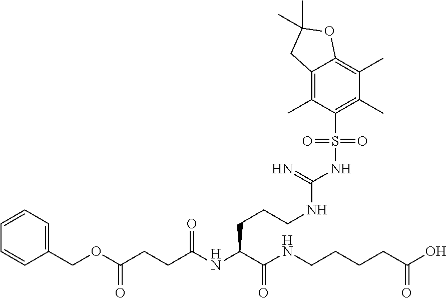 [SMILES: Cc1c(C)c(S(=O)(=O)NC(=N)NCCC[C@H](NC(=O)CCC(=O)OCc2ccccc2)C(=O)NCCCCC(=O)O)c(C)c2c1OC(C)(C)C2]

The initial resin loading with 5-((((9H-fluoren-9-yl)methoxy)carbonyl)amino)pentanoic acid was performed as described in SP-1. The Fmoc group was removed according to SP-3 and then coupled with N²-(((9H-fluoren-9-yl)methoxy)carbonyl)-N^{w}-((2,2,4,6,7-pentamethyl-2,3-dihydrobenzofuran-5-yl)sulfonyl)-L-arginine according to SP-2 but using PyBroP as the coupling reagent. The Fmoc group was removed according to SP-3 and then coupled with 4-(benzyloxy)-4-oxobutanoic acid according to SP-2. The compound was cleaved from the resin according to SP-4 to give the title compound (0.30 g, 42%). ESI/MS m/z: 716 (M).

### Preparation 99

### N2-(((9H-Fluoren-9-yl)methoxy)carbonyl)-N6-(4-(4-iodophenyl)butanoyl)-D-lysine

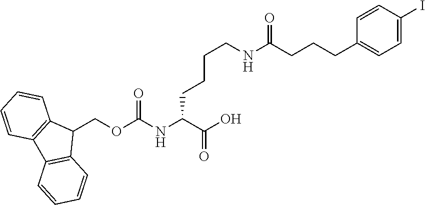 [SMILES: O=C(CCCc1ccc(I)cc1)NCCCC[C@@H](NC(=O)OCC1c2ccccc2-c2ccccc21)C(=O)O]

4-Iodophenylbutanoic acid was coupled with tert-butyl (R)-2-((((9H-fluoren-9-yl)methoxy)carbonyl)amino)-6-(λ²-azaneyl)hexanoate according to the SP-5 except using PyBrop instead of HATU to give the fully protected product. ESI/MS (m/z): 697 (M+H). The tBu group was then removed with 30% TFA in DCM to give the title compound.

### Preparation 15

### (S)-2,2,11-Trimethyl-4,7,10,13-tetraoxo-12-(3-(3-((2,2,4,5,7-pentamethyl-2,3-dihydrobenzofuran-6-yl)sulfonyl)guanidino)propyl)-3-oxa-6,11,14-triazanonadecan-19-oic acid

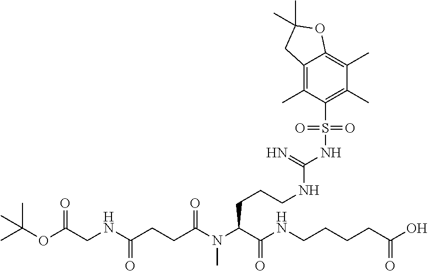 [SMILES: Cc1c(C)c(S(=O)(=O)NC(=N)NCCC[C@@H](C(=O)NCCCCC(=O)O)N(C)C(=O)CCC(=O)NCC(=O)OC(C)(C)C)c(C)c2c1OC(C)(C)C2]

The initial resin loading with 5-((((9H-fluoren-9-yl)methoxy)carbonyl)amino)pentanoic acid was performed as described in SP-1. The Fmoc group was removed according to SP-3 and then coupled with N²-methyl-N^{w}-((2,2,4,6,7-pentamethyl-2,3-dihydrobenzofuran-5-yl)sulfonyl)-L-arginine according to SP-2. The Fmoc group was removed according to SP-3 and then reacted with dihydrofuran-2,5-dione. The resulting free carboxylic acid was coupled with tert-butyl glycinate according to SP-2. The compound was cleaved from the resin according to SP-4 to give the title compound (40 mg, 54%). ESI/MS m/z: 754 (M+H).

### Preparation 16

### (S)-5-(2-(7-(tert-Butoxy)-7-oxoheptanamido)-5-(3-((2,2,4,5,7-pentamethyl-2,3-dihydrobenzofuran-6-yl)sulfonyl)guanidino)pentanamido)pentanoic acid

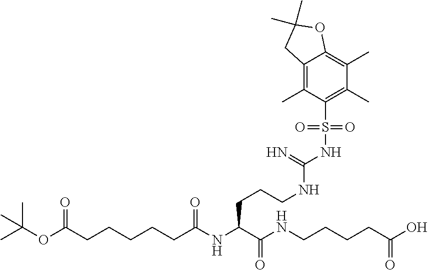 [SMILES: Cc1c(C)c(S(=O)(=O)NC(=N)NCCC[C@H](NC(=O)CCCCCC(=O)OC(C)(C)C)C(=O)NCCCCC(=O)O)c(C)c2c1OC(C)(C)C2]

The initial resin loading with 5-((((9H-fluoren-9-yl)methoxy)carbonyl)amino)pentanoic acid was performed as described in SP-1. The Fmoc group was removed according to SP-3 and then coupled with N²-(((9H-fluoren-9-yl)methoxy)carbonyl)-N^{w}-((2,2,4,6,7-pentamethyl-2,3-dihydrobenzofuran-5-yl)sulfonyl)-L-arginine according to SP-2. The Fmoc group was removed according to SP-3 and then coupled with 7-(tert-butoxy)-7-oxoheptanoic acid according to SP-2. The compound was cleaved from the resin according to SP-4 to give the title compound (45 mg, 62%). ESI/MS m/z: 724 (M).

### Preparation 17

### N²-(4-((2-(tert-Butoxy)-2-oxoethyl)amino)-4-oxobutanoyl)-N^{w}-((2,2,4,5,7-pentamethyl-2,3-dihydrobenzofuran-6-yl)sulfonyl)-L-arginine

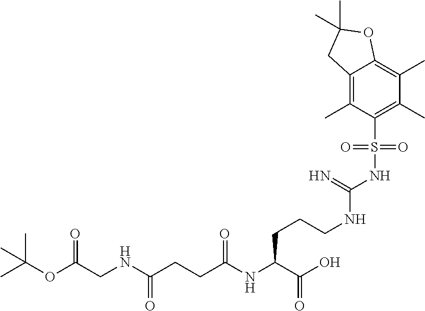 [SMILES: Cc1c(C)c(S(=O)(=O)NC(=N)NCCC[C@H](NC(=O)CCC(=O)NCC(=O)OC(C)(C)C)C(=O)O)c(C)c2c1OC(C)(C)C2]

The initial resin loading with N²-(((9H-fluoren-9-yl)methoxy)carbonyl)-N^{w}-((2,2,4,6,7-pentamethyl-2,3-dihydrobenzofuran-5-yl)sulfonyl)-L-arginine was performed as described in SP-1. The Fmoc group was removed according to SP-3 and then reacted with dihydrofuran-2,5-dione (30 mg, 0.30 mmol). The free carboxylic acid of the succinyl group was then coupled with tert-butyl glycinate according to SP-5. Afterwards, the compound was cleaved from the resin according to SP-4 to give the title compound (0.26 g, 41%). ESI/MS m/z: 641 (M+H).

### Preparation 18

### 4-(Aminomethyl)-N-(2-oxo-2-((2R)-2-((3aS,4S,6S)-3a,5,5-trimethylhexahydro-4,6-methanobenzo[d][1,3,2]dioxaborol-2-yl)pyrrolidin-1-yl)ethyl)benzamide HCl

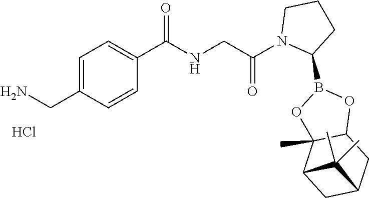

A stirred solution of 4-[(tert-butoxycarbonylamino)methyl]benzoic acid (6.0 g, 23.8 mmol) in anhydrous DMF (120 mL) was treated with HATU (9.5 g, 25 mmol) and 2-amino-1-((2R)-2-((3aS,4S,6S)-3a,5,5-trimethylhexahydro-4,6-methanobenzo[d][1,3,2]dioxaborol-2-yl)pyrrolidin-1-yl)ethan-1-one HCl (8.9 g, 25 mmol) at 0° C. DIPEA (9.1 mL, 54.7 mmol) was then added dropwise. The resulting mixture was stirred at rt for 2 h and then concentrated under reduced pressure. The residue was dissolved in EtOAc (250 mL), washed sequentially with 0.1 N aqueous KHSO₄ (3×50 mL), aqueous NaHCO₃ (3×50 mL), and saturated aqueous NaCl (3×50 mL). The organic phase was collected, dried over MgSO₄, filtered, and concentrated under reduced pressure. The protected intermediate was purified by silica gel chromatography eluted with EtOAc/hexane (10.1 g). The purified intermediate was then added to a solution of 4N HCl in 1,4-dioxane (120 mL) at 0° C. The resulting mixture was stirred at rt for 2 h and then concentrated under reduced pressure. The residue was co-evaporated with DCM (3×50 mL) under reduced pressure to give the title compound as a white powder (9.6 g, 80% over two steps).

### Preparation 19

### N-((2R)-1-((2R,4S)-4-Hydroxy-2-((3aS,4S,6S)-3a,5,5-trimethylhexahydro-4,6-methanobenzo[d][1,3,2]dioxaborol-2-yl)pyrrolidin-1-yl)-1-oxopropan-2-yl)-4-((methylamino)methyl)benzamide HCl

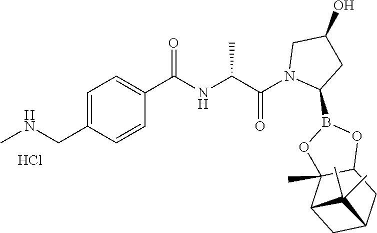

4-(((tert-Butoxycarbonyl)(methyl)amino)methyl)benzoic acid was coupled with (2R)-2-amino-1-((2R,4S)-4-hydroxy-2-((3aS,4S,6S)-3a,5,5-trimethylhexahydro-4,6-methanobenzo[d][1,3,2]dioxaborol-2-yl)pyrrolidin-1-yl)propan-1-one HCl according to SP-5 and then treated with 4N HCl in 1,4-dioxane at rt for 2 h to give the title compound (0.42 g, 81%). ESI/MS m/z: 484 (M+H).

### Preparation 20

### 4-(Aminomethyl)-N-((2R)-1-oxo-1-((2R)-2-((3aS,4S,6S)-3a,5,5-trimethylhexahydro-4,6-methanobenzo[d][1,3,2]dioxaborol-2-yl)pyrrolidin-1-yl)propan-2-yl)benzamide HCl

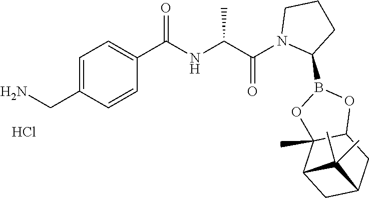

A solution of 4-[(tert-butoxycarbonylamino)methyl]benzoic acid in anhydrous DMF (8 mL) was treated with (2R)-2-amino-1-((2R)-2-((3aS,4S,6S)-3a,5,5-trimethylhexahydro-4,6-methanobenzo[d][1,3,2]dioxaborol-2-yl)pyrrolidin-1-yl)propan-1-one HCl (0.75 g, 2.1 mmol), HATU (0.80 g, 2.1 mmol), and DIPEA (0.80 mL, 4.6 mmol) at 0° C. The mixture was stirred at rt for 2 h and then concentrated under reduced pressure. The residue was dissolved in DCM (100 mL), washed sequentially with 0.1 M aqueous KHSO₄ (3×15 mL), saturated aqueous NaHCO₃ (3×15 mL), and saturated aqueous NaCl (10 mL). The organic phase was collected, dried over MgSO₄, filtered, and concentrated under reduced pressure. The residue was purified by silica gel chromatography eluted with 1:1 EtOAc in hexanes. The collected fractions were cooled to 0° C. and treated with 4N HCl in 1,4-dioxane (10 mL). The resulting mixture was stirred at rt for 2 h and then concentrated under reduced pressure. The residue was co-evaporated with dichloromethane (3×20 mL) to give the title compound (830 mg, 85% over two steps) as a white powder. ESI/MS m/z: 454 (M+H).

### Preparation 21

### 4-((5-(Methylamino)pentanamido)methyl)-N-((2R)-1-oxo-1-((2R)-2-((3aS,4S,6S)-3a,5,5-trimethylhexahydro-4,6-methanobenzo[d][1,3,2]dioxaborol-2-yl)pyrrolidin-1-yl)propan-2-yl)benzamide

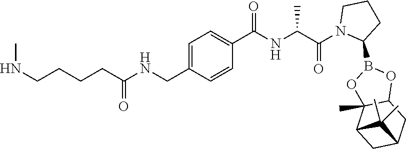

4-(Aminomethyl)-N-((2R)-1-oxo-1-((2R)-2-((3aS,4S,6S)-3a,5,5-trimethylhexahydro-4,6-methanobenzo[d][1,3,2]dioxaborol-2-yl)pyrrolidin-1-yl)propan-2-yl)benzamide HCl was coupled to 5-((tert-butoxycarbonyl)(methyl)amino)pentanoic acid with HATU and DIPEA in DMF according to SP-5 (working on a 0.5 mmol scale). The Boc protecting group was removed with 4N HCl in 1,4-dioxane to give the title compound (0.27 g, 90%). ESI/MS m/z: 567 (M+H).

### Preparation 22

### tert-Butyl (5-((4-(((2R)-1-((2R,4S)-4-hydroxy-2-((3aS,4S,6S)-3a,5,5-trimethylhexahydro-4,6-methanobenzo[d][1,3,2]dioxaborol-2-yl)pyrrolidin-1-yl)-1-oxopropan-2-yl)carbamoyl)benzyl)(methyl)amino)-5-oxopentyl)(methyl)carbamate

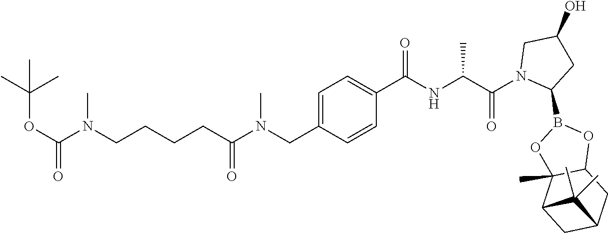

4-(((tert-Butoxycarbonyl)(methyl)amino)methyl)benzoic acid was coupled to (2R)-2-amino-1-((2R,4S)-4-hydroxy-2-((3aS,4S,6S)-3a,5,5-trimethylhexahydro-4,6-methanobenzo[d][1,3,2]dioxaborol-2-yl)pyrrolidin-1-yl)propan-1-one according to SP-5 (working on a 0.5 mmol scale) and then the Boc group was removed with 4N HCl in 1,4-dioxane. The free amine was coupled to 5-((tert-butoxycarbonyl)(methyl)amino)pentanoic acid according to SP-5 to give the title compound (0.25 g, 72%). ESI/MS m/z: 698 (M+H).

### Preparation 23

### tert-Butyl N⁶-(tert-butoxycarbonyl)-N²-((4-(((2S)-1-(methyl(5-oxo-5-((4-(((2R)-1-oxo-1-((2R)-2-((3aS,4S,6S)-3a,5,5-trimethylhexahydro-4,6-methanobenzo[d][1,3,2]dioxaborol-2-yl)pyrrolidin-1-yl)propan-2-yl)carbamoyl)benzyl)amino)pentyl)amino)-1-oxo-5-(3-((2,2,4,5,7-pentamethyl-2,3-dihydrobenzofuran-6-yl)sulfonyl)guanidino)pentan-2-yl)amino)-4-oxobutanoyl)glycyl)-L-lysinate

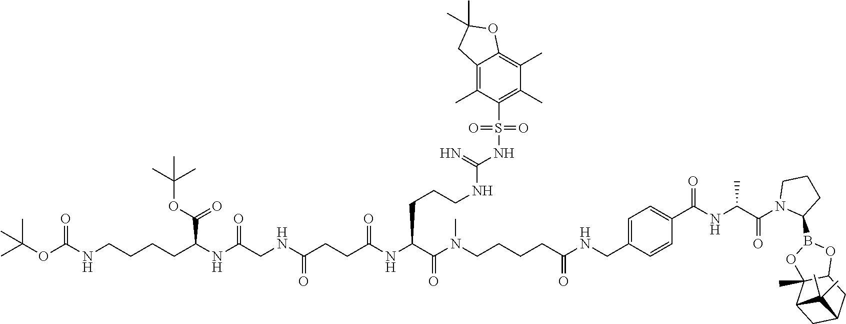

N²-(4-((2-(tert-Butoxy)-2-oxoethyl)amino)-4-oxobutanoyl)-N^{w}-((2,2,4,6,7-pentamethyl-2,3-dihydrobenzofuran-5-yl)sulfonyl)-L-arginine and 4-((5-(methylamino)pentanamido)methyl)-N-((2R)-1-oxo-1-((2R)-2-((3aS,4S,6S)-3a,5,5-trimethylhexahydro-4,6-methanobenzo[d][1,3,2]dioxaborol-2-yl)pyrrolidin-1-yl)propan-2-yl)benzamide were coupled according to SP-5 (working on a 0.25 mmol scale) but using PyBroP as the coupling reagent. The tert-butyl ester group was removed with 4N HCl in 1,4-dioxane and then coupled to tert-butyl N⁶-(tert-butoxycarbonyl)-L-lysinate with HATU and DIPEA in DMF to give the title compound (0.30 g, 85%). ESI/MS m/z: 1418 (M+H).

### Preparation 24

### tert-Butyl 7-(((8S)-3-imino-2-methyl-9,15-dioxo-17-(4-(((2R)-1-oxo-1-((2R)-2-((3aS,4S,6S)-3a,5,5-trimethylhexahydro-4,6-methanobenzo[d][1,3,2]dioxaborol-2-yl)pyrrolidin-1-yl)propan-2-yl)carbamoyl)phenyl)-2,4,10,16-tetraazaheptadecan-8-yl)amino)-7-oxoheptanoate

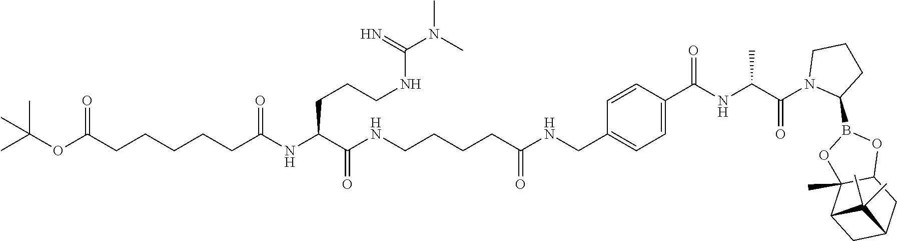

The initial resin loading with 5-((((9H-fluoren-9-yl)methoxy)carbonyl)amino)pentanoic acid was performed as described in SP-1. The Fmoc group was removed according to SP-3 and then coupled with N²-(((9H-fluoren-9-yl)methoxy)carbonyl)-N^{w},N^{w}-dimethyl-L-arginine according to SP-2. The Fmoc group was removed according to SP-3 and then coupled with 7-(tert-butoxy)-7-oxoheptanoic acid according to SP-2. The compound was cleaved from the resin according to SP-4 and then coupled to 4-(aminomethyl)-N-((2R)-1-oxo-1-((2R)-2-((3aS,4S,6S)-3a,5,5-trimethylhexahydro-4,6-methanobenzo[d][1,3,2]dioxaborol-2-yl)pyrrolidin-1-yl)propan-2-yl)benzamide according to SP-5 to give the title compound (0.33 g, 36%). ESI/MS m/z: 936 (M+H).

### Preparation 25

### (S)-5-(5-(3-((2,2,4,5,7-Pentamethyl-2,3-dihydrobenzofuran-6-yl)sulfonyl)guanidino)-2-(2-(4,7,10-tris(2-(tert-butoxy)-2-oxoethyl)-1,4,7,10-tetraazacyclododecan-1-yl)acetamido)pentanamido)pentanoic acid

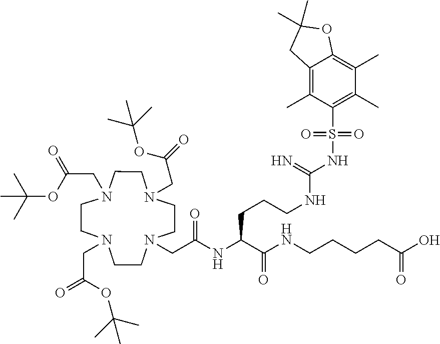 [SMILES: Cc1c(C)c(S(=O)(=O)NC(=N)NCCC[C@H](NC(=O)CN2CCN(CC(=O)OC(C)(C)C)CCN(CC(=O)OC(C)(C)C)CCN(CC(=O)OC(C)(C)C)CC2)C(=O)NCCCCC(=O)O)c(C)c2c1OC(C)(C)C2]

The initial resin loading with 5-((((9H-fluoren-9-yl)methoxy)carbonyl)amino)pentanoic acid was performed as described in SP-1 (working on a 0.5 mmol scale). The Fmoc group was removed according to SP-3 and then coupled with N²-(((9H-fluoren-9-yl)methoxy)carbonyl)-N^{w}-((2,2,4,6,7-pentamethyl-2,3-dihydrobenzofuran-5-yl)sulfonyl)-L-arginine according to SP-2 but using PyBroP as the coupling reagent. The Fmoc group was removed according to SP-3 and then coupled with 2-(4,7,10-tris(2-(tert-butoxy)-2-oxoethyl)-1,4,7,10-tetraazacyclododecan-1-yl)acetic acid according to SP-2 but using PyBroP as the coupling reagent. The compound was cleaved from the resin according to SP-4 to give the title compound (0.16 g, 30%). ESI/MS m/z: 1081 (M+H).

### Preparation 26

### (S)-4-((5-(2-((((9H-Fluoren-9-yl)methoxy)carbonyl)amino)-5-(3-((2,2,4,6,7-pentamethyl-2,3-dihydrobenzofuran-5-yl)sulfonyl)guanidino)pentanamido)pentyl)carbamoyl)-1-naphthoic acid

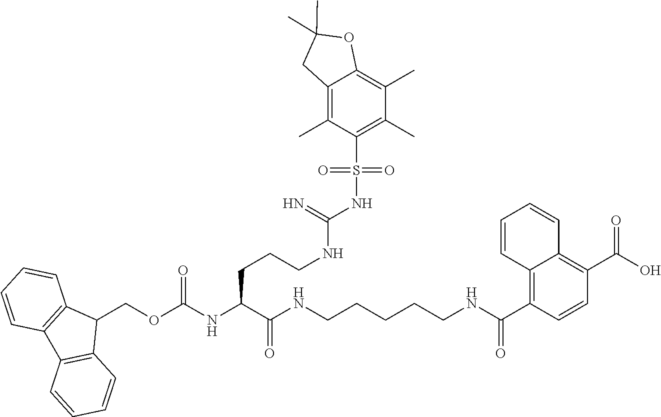 [SMILES: Cc1c(C)c(S(=O)(=O)NC(=N)NCCC[C@H](NC(=O)OCC2c3ccccc3-c3ccccc32)C(=O)NCCCCCNC(=O)c2ccc(C(=O)O)c3ccccc23)c(C)c2c1OC(C)(C)C2]

The initial resin loading with naphthalene-1,4-dicarboxylic acid was performed as described in SP-1 (working on a 0.5 mmol scale) and coupled with (9H-fluoren-9-yl)methyl (5-aminopentyl)carbamate HCl according to SP-2. The Fmoc group was removed according to SP-3 and then coupled with (S)-2-((((9H-fluoren-9-yl)methoxy)carbonyl)amino)-5-((((2,2,4,6,7-pentamethyl-2,3-dihydrobenzofuran-5-yl)sulfonyl)-l²-azaneyl)formimidamido)pentanoic acid according to SP-2 using PyBroP as the coupling reagent. The compound was cleaved from the resin according to SP-4 to give the title compound (0.15 g, 32%).

### Preparation 27

### (S)-4-(1-(9H-Fluoren-9-yl)-3,6,12-trioxo-5-(3-(3-((2,2,4,6,7-pentamethyl-2,3-dihydrobenzofuran-5-yl)sulfonyl)guanidino)propyl)-2-oxa-4,7,13-triazatetradecan-14-yl)benzoic acid

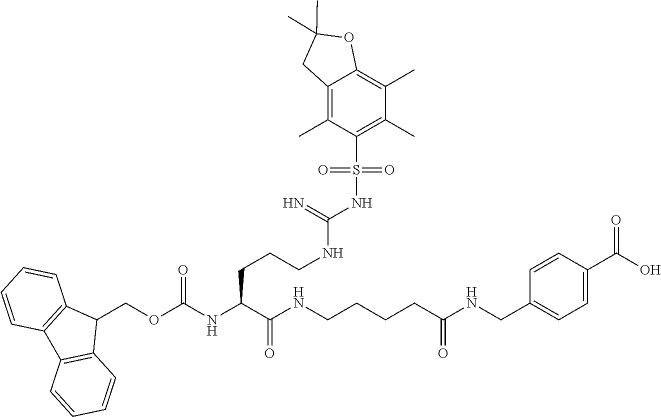 [SMILES: Cc1c(C)c(S(=O)(=O)NC(=N)NCCC[C@H](NC(=O)OCC2c3ccccc3-c3ccccc32)C(=O)NCCCCC(=O)NCc2ccc(C(=O)O)cc2)c(C)c2c1OC(C)(C)C2]

The initial resin loading with 4-(((((9H-fluoren-9-yl)methoxy)carbonyl)amino)methyl)benzoic acid was performed as described in SP-1 (working on a 0.5 mmol scale). The Fmoc group was removed according to SP-3 and then coupled with 5-((((9H-fluoren-9-yl)methoxy)carbonyl)amino)pentanoic acid according to SP-2. The Fmoc group was removed according to SP-3 and then coupled with N²-(((9H-fluoren-9-yl)methoxy)carbonyl)-N^{w}-((2,2,4,6,7-pentamethyl-2,3-dihydrobenzofuran-5-yl)sulfonyl)-L-arginine according to SP-2. The compound was cleaved from the resin according to SP-4 to give the title compound (0.16 g, 40%). ESI/MS m/z: 882 (M+H).

### Preparation 28

### (S)-4-((5-(2-(7-(tert-Butoxy)-7-oxoheptanamido)-5-(3-((2,2,4,6,7-pentamethyl-2,3-dihydrobenzofuran-5-yl)sulfonyl)guanidino)pentanamido)pentyl)carbamoyl)-1-naphthoic acid

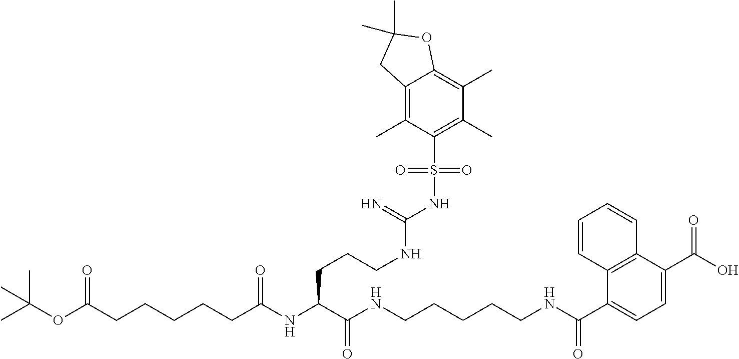 [SMILES: Cc1c(C)c(S(=O)(=O)NC(=N)NCCC[C@H](NC(=O)CCCCCC(=O)OC(C)(C)C)C(=O)NCCCCCNC(=O)c2ccc(C(=O)O)c3ccccc23)c(C)c2c1OC(C)(C)C2]

N²-(((9H-Fluoren-9-yl)methoxy)carbonyl)-N^{w}-((2,2,4,6,7-pentamethyl-2,3-dihydrobenzofuran-5-yl)sulfonyl)-L-arginine was coupled to tert-butyl (5-aminopentyl)carbamate using HATU and DIPEA in DMF. The Boc group was removed, and the remaining amine coupled to the preloaded naphthalene-1,4-dicarboxylic acid-2-Cl-Trt-resin according to SP-2. The Fmoc group was removed according to SP-3 and then coupled to 7-(tert-butoxy)-7-oxoheptanoic acid according to SP-2. The compound was cleaved from the resin according to SP-4 to give the title compound (0.16 g, 35%). ESI/MS m/z: 908 (M+H).

### Preparation 29

### (S,Z)-4-((5-(2-(7-(tert-Butoxy)-7-oxoheptanamido)-5-(((dimethylamino)((2,2,4,6,7-pentamethyl-2,3-dihydrobenzofuran)-5-sulfonamido)methylene)amino)pentanamido)pentyl)carbamoyl)-1-naphthoic acid

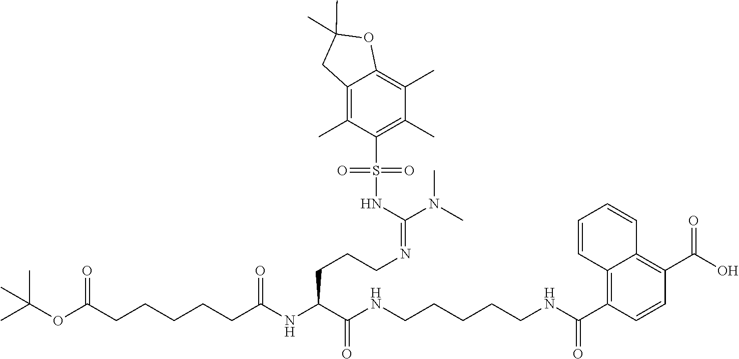 [SMILES: Cc1c(C)c(S(=O)(=O)N/C(=N\CCC[C@H](NC(=O)CCCCCC(=O)OC(C)(C)C)C(=O)NCCCCCNC(=O)c2ccc(C(=O)O)c3ccccc23)N(C)C)c(C)c2c1OC(C)(C)C2]

The initial resin loading with naphthalene-1,4-dicarboxylic acid was performed as described in SP-1 (working on a 0.5 mmol scale) and then coupled with (9H-fluoren-9-yl)methyl (5-aminopentyl)carbamate HCl according to SP-2 using PyBroP instead of HATU as the coupling reagent. The Fmoc group was removed according to SP-3 and then coupled with (S,Z)-2-((((9H-fluoren-9-yl)methoxy)carbonyl)amino)-5-(((dimethylamino)((2,2,4,6,7-pentamethyl-2,3-dihydrobenzofuran)-5-sulfonamido)methylene)amino)pentanoic acid according to SP-2 using PyBroP instead of HATU as the coupling reagent. The Fmoc group was removed according to SP-3 and then coupled to 6-(tert-butoxy)-6-oxohexanoic acid according to SP-2 using PyBroP instead of HATU as the coupling reagent. The compound was cleaved from the resin according to SP-4 to give the title compound (0.14 g, 29%) ESI/MS m/z: 936 (M+H).

### Preparation 30

### (S,Z)-4-((20-(3-(3,3-dimethyl-2-((2,2,4,6,7-pentamethyl-2,3-dihydrobenzofuran-5-yl)sulfonyl)guanidino)propyl)-2,2-dimethyl-4,12,18,21-tetraoxo-3-oxa-5,11,19,22-tetraazaheptacosan-27-yl)carbamoyl)-1-naphthoic acid

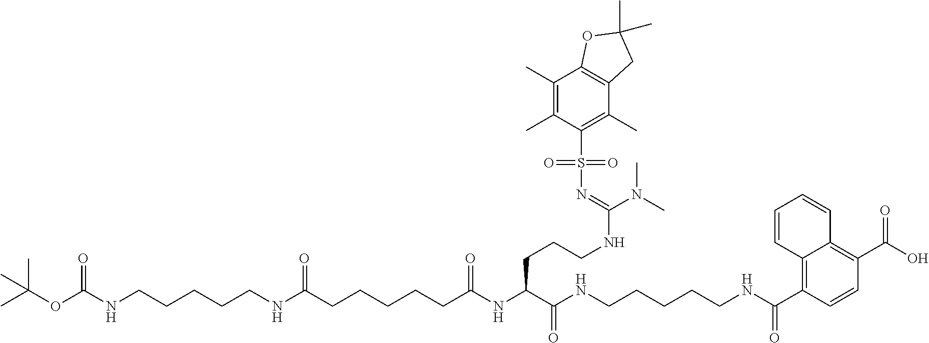 [SMILES: Cc1c(C)c(S(=O)(=O)/N=C(/NCCC[C@H](NC(=O)CCCCCC(=O)NCCCCCNC(=O)OC(C)(C)C)C(=O)NCCCCCNC(=O)c2ccc(C(=O)O)c3ccccc23)N(C)C)c(C)c2c1OC(C)(C)C2]

The initial resin loading with naphthalene-1,4-dicarboxylic acid was performed as described in SP-1 (working on a 0.1 mmol scale) and then coupled with (9H-fluoren-9-yl)methyl (5-aminopentyl)carbamate HCl according to SP-2 with PyBroP instead of HATU as the coupling reagent. The Fmoc group was removed according to SP-3 and then coupled with (S,Z)-2-((((9H-fluoren-9-yl)methoxy)carbonyl)amino)-5-(((dimethylamino)((2,2,4,6,7-pentamethyl-2,3-dihydrobenzofuran)-5-sulfonamido)methylene)amino)pentanoic acid according to SP-2. The Fmoc group was removed according to SP-3 and then coupled with 7-(4-nitrophenoxy)-7-oxoheptanoic acid in DMF with DIPEA. The free carboxylic acid was then coupled to N-Boc-1,5-diaminopentane according to SP-2. The compound was cleaved from the resin according to SP-4 to give the title compound (0.03 g, 28%). ESI/MS m/z: 1064 (M+H).

### Preparation 31

### (S,Z)-4-(10-(3-(3,3-Dimethyl-2-((2,2,4,6,7-pentamethyl-2,3-dihydrobenzofuran-5-yl)sulfonyl)guanidino)propyl)-28,28-dimethyl-3,9,12,18,26-pentaoxo-27-oxa-2,8,11,19,25-pentaazanonacosyl)benzoic acid

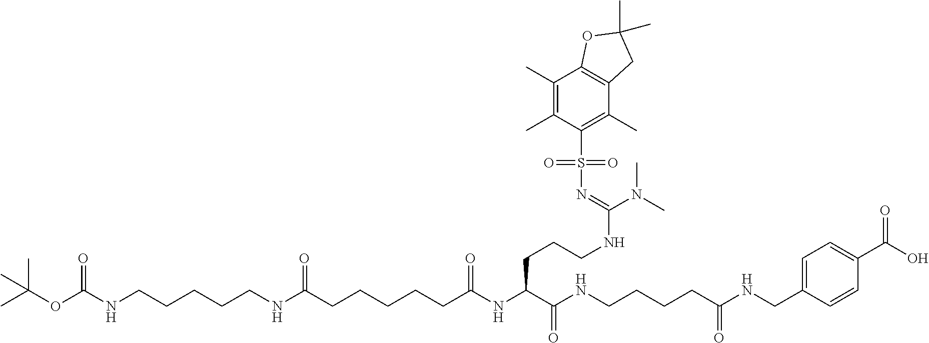 [SMILES: Cc1c(C)c(S(=O)(=O)/N=C(/NCCC[C@H](NC(=O)CCCCCC(=O)NCCCCCNC(=O)OC(C)(C)C)C(=O)NCCCCC(=O)NCc2ccc(C(=O)O)cc2)N(C)C)c(C)c2c1OC(C)(C)C2]

The initial resin loading with 4-(((((9H-fluoren-9-yl)methoxy)carbonyl)amino)methyl)benzoic acid was performed as described in SP-1. The Fmoc group was removed according to SP-3 and then coupled with 5-((((9H-fluoren-9-yl)methoxy)carbonyl)amino)pentanoic acid according to SP-2. The Fmoc group was removed according to SP-3 and then coupled with (S,Z)-2-((((9H-fluoren-9-yl)methoxy)carbonyl)amino)-5-(((dimethylamino)((2,2,4,6,7-pentamethyl-2,3-dihydrobenzofuran)-5-sulfonamido)methylene)amino)pentanoic acid according to SP-2. The Fmoc group was removed according to SP-3 and then coupled with 7-(4-nitrophenoxy)-7-oxoheptanoic acid in DMF with DIPEA. The free carboxylic acid was then coupled to tert-butyl (5-aminopentyl)carbamate according to SP-2. The compound was cleaved from the resin according to SP-4 to give the title compound (0.035 g, 35%). ESI/MS m/z: 1014 (M+H).

### Preparation 32

### (S,Z)-4-(8-Amino-2-methyl-9,15-dioxo-3-((2,2,4,6,7-pentamethyl-2,3-dihydrobenzofuran)-5-sulfonamido)-2,4,10,16-tetraazaheptadec-3-en-17-yl)cyclohexane-1-carboxylic acid

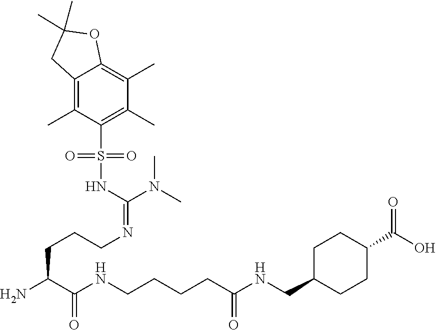 [SMILES: Cc1c(C)c(S(=O)(=O)N/C(=N\CCC[C@H](N)C(=O)NCCCCC(=O)NC[C@H]2CC[C@H](C(=O)O)CC2)N(C)C)c(C)c2c1OC(C)(C)C2]

The initial resin loading with trans-4-(((((9H-fluoren-9-yl)methoxy)carbonyl)amino)methyl)cyclohexane-1-carboxylic acid was performed according to SP-1 (working on a 0.5 mmol scale). The Fmoc group was removed according to SP-3 and then coupled with 5-((((9H-fluoren-9-yl)methoxy)carbonyl)amino)pentanoic acid according to SP-2 using PyBroP as the coupling reagent. The Fmoc group was removed according to SP-3 and then coupled with (S,Z)-2-((((9H-fluoren-9-yl)methoxy)carbonyl)amino)-5-(((dimethylamino)((2,2,4,6,7-pentamethyl-2,3-dihydrobenzofuran)-5-sulfonamido)methylene)amino)pentanoic acid according to SP-2 using PyBroP as the coupling reagent. The Fmoc group was removed according to SP-3. The compound was cleaved from the resin according to SP-4 to give the title compound (0.20 g, 50%). ESI/MS m/z: 694 (M+H).

### Preparation 33

### N¹-(5-((S)-2-Amino-5-(3-((2,2,4,5,7-pentamethyl-2,3-dihydrobenzofuran-6-yl)sulfonyl)guanidino)pentanamido)pentyl)-N⁴-((2R)-1-oxo-1-((2R)-2-((3aS,4S,6S)-3a,5,5-trimethylhexahydro-4,6-methanobenzo[d][1,3,2]dioxaborol-2-yl)pyrrolidin-1-yl)propan-2-yl)naphthalene-1,4-dicarboxamide

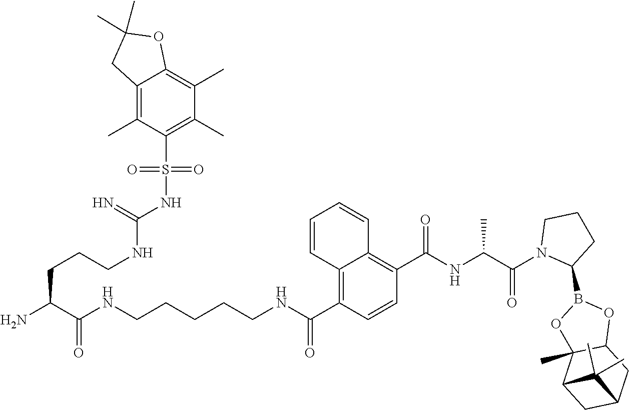

(S)-4-((5-(2-((((9H-Fluoren-9-yl)methoxy)carbonyl)amino)-5-(3-((2,2,4,6,7-pentamethyl-2,3-dihydrobenzofuran-5-yl)sulfonyl)guanidino)pentanamido)pentyl)carbamoyl)-1-naphthoic acid was coupled with (2R)-2-amino-1-((2R)-2-((3aS,4S,6S)-3a,5,5-trimethylhexahydro-4,6-methanobenzo[d][1,3,2]dioxaborol-2-yl)pyrrolidin-1-yl)propan-1-one HCl according to SP-5 (working on a 0.25 mmol scale). The Fmoc group was removed with 20% piperidine in DMF. The crude product was further purified using the General Procedure for Preparative HPLC purification (Eluent gradient: 20% B for the first 3 min, then from 20% to 98% B over 12 min, which was maintained for the next 5 min). Both peaks of the desired compound and de-pinanediol compound were collected, concentrated under reduced pressure, and lyophilized to give the title compound (0.18 g, 65%). ESI/MS m/z: 1012 (M+H).

### Preparation 34

### 4-((5-((S)-2-Amino-5-(3-((2,2,4,6,7-pentamethyl-2,3-dihydrobenzofuran-5-yl)sulfonyl)guanidino)pentanamido)pentanamido)methyl)-N-((2R)-1-((2R,4S)-4-hydroxy-2-((3aS,4S,6S)-3a,5,5-trimethylhexahydro-4,6-methanobenzo[d][1,3,2]dioxaborol-2-yl)pyrrolidin-1-yl)-1-oxopropan-2-yl)benzamide

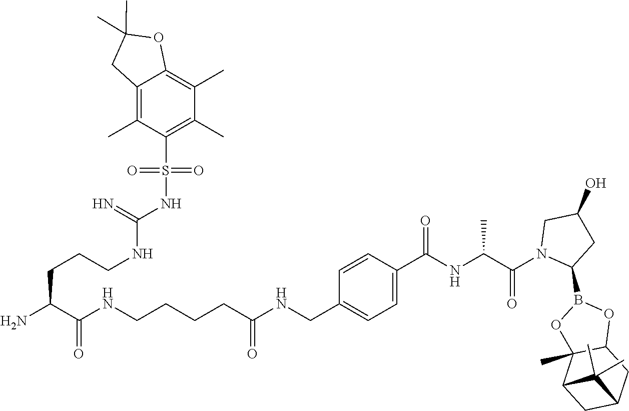

(S)-4-(1-(9H-Fluoren-9-yl)-3,6,12-trioxo-5-(3-(3-((2,2,4,6,7-pentamethyl-2,3-dihydrobenzofuran-5-yl)sulfonyl)guanidino)propyl)-2-oxa-4,7,13-triazatetradecan-14-yl)benzoic acid was coupled to (2R)-2-amino-1-((2R,4S)-4-hydroxy-2-((3aS,4S,6S)-3a,5,5-trimethylhexahydro-4,6-methanobenzo[d][1,3,2]dioxaborol-2-yl)pyrrolidin-1-yl)propan-1-one HCl according to SP-5 (working on a 0.25 mmol scale) and the Fmoc group removed with 5% piperidine in DMF to give the title compound (0.20 g, 73%). ESI/MS m/z: 978 (M+H).

### Preparation 35

### 4-((5-((S)-2-Amino-N-methyl-5-(3-((2,2,4,6,7-pentamethyl-2,3-dihydrobenzofuran-5-yl)sulfonyl)guanidino)pentanamido)-N-methylpentanamido)methyl)-N-((2R)-1-((2R,4S)-4-hydroxy-2-((3aS,4S,6S)-3a,5,5-trimethylhexahydro-4,6-methanobenzo[d][1,3,2]dioxaborol-2-yl)pyrrolidin-1-yl)-1-oxopropan-2-yl)benzamide

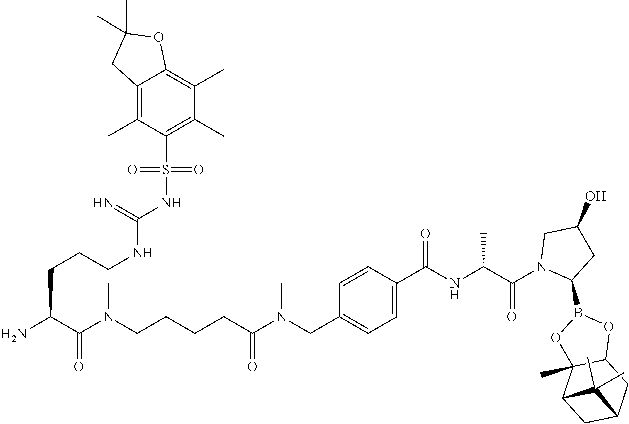

tert-Butyl (5-((4-(((2R)-1-((2R,4S)-4-hydroxy-2-((3aS,4S,6S)-3a,5,5-trimethylhexahydro-4,6-methanobenzo[d][1,3,2]dioxaborol-2-yl)pyrrolidin-1-yl)-1-oxopropan-2-yl)carbamoyl)benzyl)(methyl)amino)-5-oxopentyl)(methyl)carbamate was deprotected with 4N HCl in 1,4-dioxane (working on 0.25 mmol scale). The free amine was coupled to N²-(((9H-fluoren-9-yl)methoxy)carbonyl)-N^{w}-((2,2,4,6,7-pentamethyl-2,3-dihydrobenzofuran-5-yl)sulfonyl)-L-arginine according to SP-5. The Fmoc was removed with 20% piperidine in DMF to give the title compound (0.20 g, 70%). ESI/MS m/z: 1006 (M+H).

### Preparation 36

### 4-Oxo-4-(((2S)-1-oxo-1-((5-oxo-5-((4-(((2R)-1-oxo-1-((2R)-2-((3aS,4S,6S)-3a,5,5-trimethylhexahydro-4,6-methanobenzo[d][1,3,2]dioxaborol-2-yl)pyrrolidin-1-yl)propan-2-yl)carbamoyl)benzyl)amino)pentyl)amino)-5-(3-((2,2,4,5,7-pentamethyl-2,3-dihydrobenzofuran-6-yl)sulfonyl)guanidino)pentan-2-yl)amino)butanoic acid

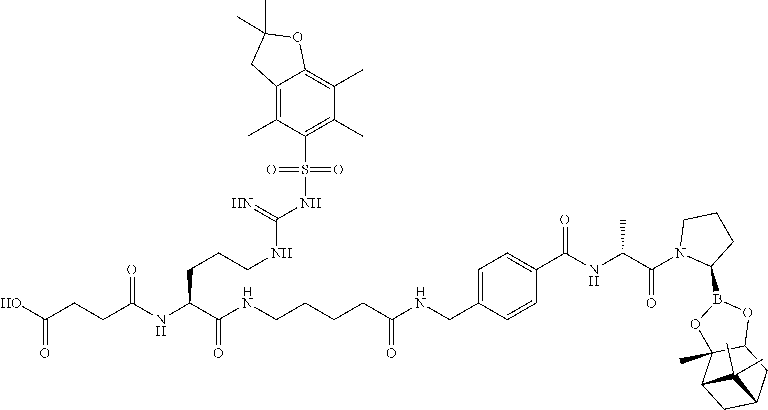

(S)-5-(2-(4-(Benzyloxy)-4-oxobutanamido)-5-(3-((2,2,4,6,7-pentamethyl-2,3-dihydrobenzofuran-5-yl)sulfonyl)guanidino)pentanamido)pentanoic acid was coupled with 4-((l²-azaneyl)methyl)-N-((2R)-1-oxo-1-((2R)-2-((3aS,4S,6S)-3a,5,5-trimethylhexahydro-4,6-methanobenzo[d][1,3,2]dioxaborol-2-yl)pyrrolidin-1-yl)propan-2-yl)benzamide HCl according to the SP-5 (working on a 0.25 mmol scale). The Bn protective group was then removed under an H₂ balloon with 10% Pd—C in MeOH to give the title compound (0.23 g, 87%). ESI/MS m/z: 1062 (M+H).

### Preparation 37

### 4-((5-((S)-2-Amino-5-(3-((2,2-dimethyl-2,3-dihydrobenzofuran-5-yl)sulfonyl)guanidino)pentanamido)pentanamido)methyl)-N-((2R)-1-oxo-1-((2R)-2-((3aS,4S,6S)-3a,5,5-trimethylhexahydro-4,6-methanobenzo[d][1,3,2]dioxaborol-2-yl)pyrrolidin-1-yl)propan-2-yl)benzamide

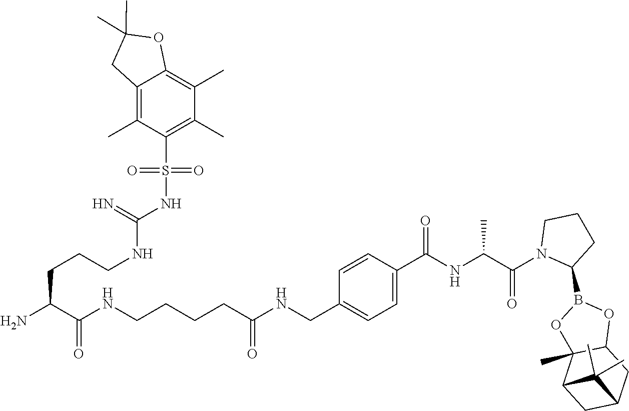

(S)-4-(5-(3-(3-((2,2-Dimethyl-2,3-dihydrobenzofuran-5-yl)sulfonyl)guanidino)propyl)-1-(9H-fluoren-9-yl)-3,6,12-trioxo-2-oxa-4,7,13-triazatetradecan-14-yl)benzoic acid was coupled to (2R)-2-amino-1-((2R)-2-((3aS,4S,6S)-3a,5,5-trimethylhexahydro-4,6-methanobenzo[d][1,3,2]dioxaborol-2-yl)pyrrolidin-1-yl)propan-1-one HCl according to SP-5 (working on a 0.25 mmol scale). The Fmoc group was removed with 20% piperidine in DMF to give the title compound (0.18 g, 67%). ESI/MS m/z: 962 (M+H).

### Preparation 38

### 7-(((8S,Z)-17-(4-(((2R)-1-((2R,4S)-4-Hydroxy-2-((3aS,4S,6S)-3a,5,5-trimethylhexahydro-4,6-methanobenzo[d][1,3,2]dioxaborol-2-yl)pyrrolidin-1-yl)-1-oxopropan-2-yl)carbamoyl)naphthalen-1-yl)-2-methyl-9,17-dioxo-3-((2,2,4,6,7-pentamethyl-2,3-dihydrobenzofuran)-5-sulfonamido)-2,4,10,16-tetraazaheptadec-3-en-8-yl)amino)-7-oxoheptanoic acid

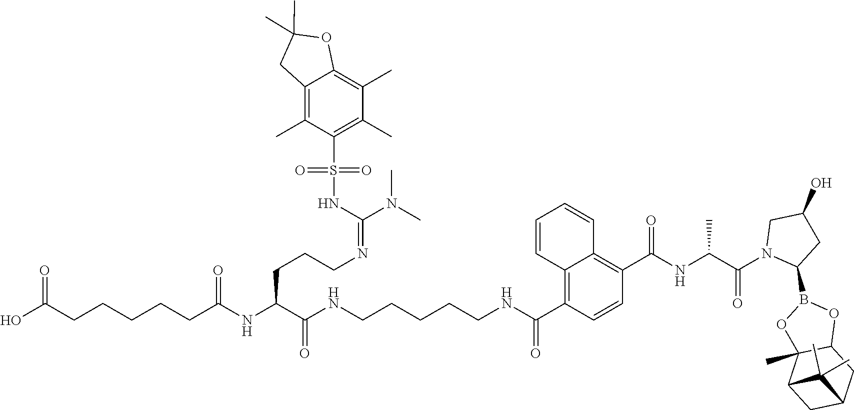

(S,Z)-4-((5-(2-(7-(tert-Butoxy)-7-oxoheptanamido)-5-(((dimethylamino)((2,2,4,6,7-pentamethyl-2,3-dihydrobenzofuran)-5-sulfonamido)methylene)amino)pentanamido)pentyl)carbamoyl)-1-naphthoic acid was coupled to (2R)-2-amino-1-((2R,4S)-4-hydroxy-2-((3aS,4S,6S)-3a,5,5-trimethylhexahydro-4,6-methanobenzo[d][1,3,2]dioxaborol-2-yl)pyrrolidin-1-yl)propan-1-one according to the SP-5 (working on a 0.15 mmol scale). The tert-butyl ester protecting group was removed with 4N HCl in 1,4-dioxane to give the title compound (0.13 g, 72%). ESI/MS m/z: 1198 (M+H).

### Preparation 39

### ((R)-1-((4-((5-Aminopentanamido)methyl)benzoyl)-D-alanyl)pyrrolidin-2-yl)boronic acid

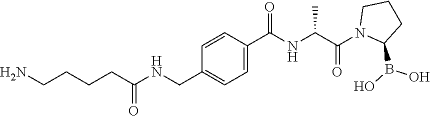 [SMILES: C[C@@H](NC(=O)c1ccc(CNC(=O)CCCCN)cc1)C(=O)N1CCC[C@H]1B(O)O]

5-((tert-Butoxycarbonyl)amino)pentanoic acid was coupled to 4-((l²-azaneyl)methyl)-N-((2R)-1-oxo-1-((2R)-2-((3aS,4S,6S)-3a,5,5-trimethylhexahydro-4,6-methanobenzo[d][1,3,2]dioxaborol-2-yl)pyrrolidin-1-yl)propan-2-yl)benzamide according to SP-5 (working on a 0.5 mmol scale) and then reacted with 4N HCl in 1,4-dioxane and treated with water to give the title compound (0.34 g, 72%). ESI/MS m/z: 419 (M+H).

### Preparation 40

### ((R)-1-((4-((S)-8-(5-Aminopentanamido)-3-imino-2-methyl-9,15-dioxo-2,4,10,16-tetraazaheptadecan-17-yl)benzoyl)-D-alanyl)pyrrolidin-2-yl)boronic acid

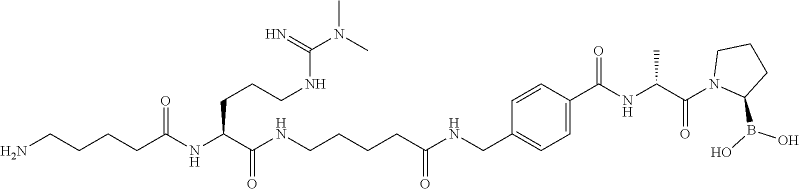 [SMILES: C[C@@H](NC(=O)c1ccc(CNC(=O)CCCCNC(=O)[C@H](CCCNC(=N)N(C)C)NC(=O)CCCCN)cc1)C(=O)N1CCC[C@H]1B(O)O]

((R)-1-((4-((5-Aminopentanamido)methyl)benzoyl)-D-alanyl)pyrrolidin-2-yl)boronic acid was coupled with N²-(((9H-fluoren-9-yl)methoxy)carbonyl)-N^{w},N^{w}-dimethyl-L-arginine according to SP-5 (working on a 0.25 mmol scale). The Fmoc group was removed with 20% piperidine in DMF, coupled with 5-((tert-butoxycarbonyl)amino)pentanoic acid according to SP-5, and the Boc group removed with 4N HCl in 1,4-dioxane to give the title compound (0.09 g, 46%). ESI/MS m/z: 703 (M+H).

### Preparation 41

### (S)-4-((5-(2-(2-(7-(2-Amino-2-oxoethyl)-4,10-bis(2-(tert-butoxy)-2-oxoethyl)-1,4,7,10-tetraazacyclododecan-1-yl)acetamido)-5-(3-((2,2,4,5,7-pentamethyl-2,3-dihydrobenzofuran-6-yl)sulfonyl)guanidino)pentanamido)-N-methylpentanamido)methyl)benzoic acid

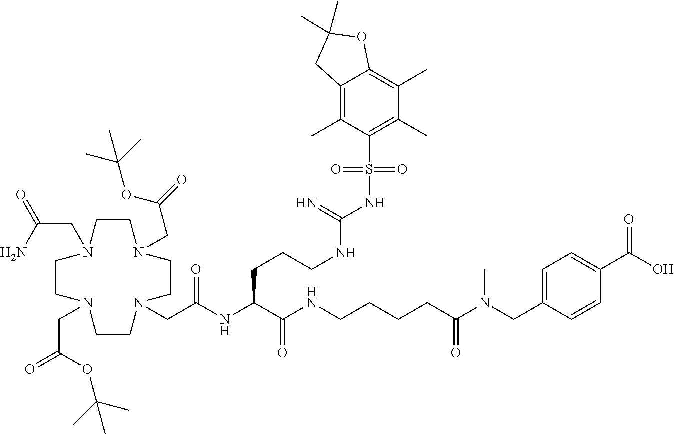 [SMILES: Cc1c(C)c(S(=O)(=O)NC(=N)NCCC[C@H](NC(=O)CN2CCN(CC(=O)OC(C)(C)C)CCN(CC(N)=O)CCN(CC(=O)OC(C)(C)C)CC2)C(=O)NCCCCC(=O)N(C)Cc2ccc(C(=O)O)cc2)c(C)c2c1OC(C)(C)C2]

The initial resin loading with 4-(((((9H-fluoren-9-yl)methoxy)carbonyl)(methyl)amino)methyl)benzoic acid was performed as described in SP-1 (working on a 0.1 mmol scale). The Fmoc group was removed according to SP-3 and then coupled with 5-((((9H-fluoren-9-yl)methoxy)carbonyl)amino)pentanoic acid according to SP-2. The Fmoc group was removed according to SP-3 and coupled with N²-(((9H-fluoren-9-yl)methoxy)carbonyl)-N^{w}-((2,2,4,6,7-pentamethyl-2,3-dihydrobenzofuran-5-yl)sulfonyl)-L-arginine according to SP-2 but using PyBroP as the coupling reagent. The amino group was deprotected according to SP-3 and then coupled with 2-(7-(2-amino-2-oxoethyl)-4,10-bis(2-(tert-butoxy)-2-oxoethyl)-1,4,7,10-tetraazacyclododecan-1-yl)acetic acid according to SP-2. The peptide was cleaved from the resin according to SP-4 to give the title compound (0.04 g, 30%). ESI/MS m/z: 1171 (M+H).

### Preparation 42

### (S,Z)-4-(2-Methyl-9,15-dioxo-3-(((2,2,4,5,7-pentamethyl-2,3-dihydrobenzofuran-6-yl)sulfonyl)imino)-8-(2-(4,7,10-tris(2-(tert-butoxy)-2-oxoethyl)-1,4,7,10-tetraazacyclododecan-1-yl)acetamido)-2,4,10,16-tetraazaheptadecan-17-yl)benzoic acid

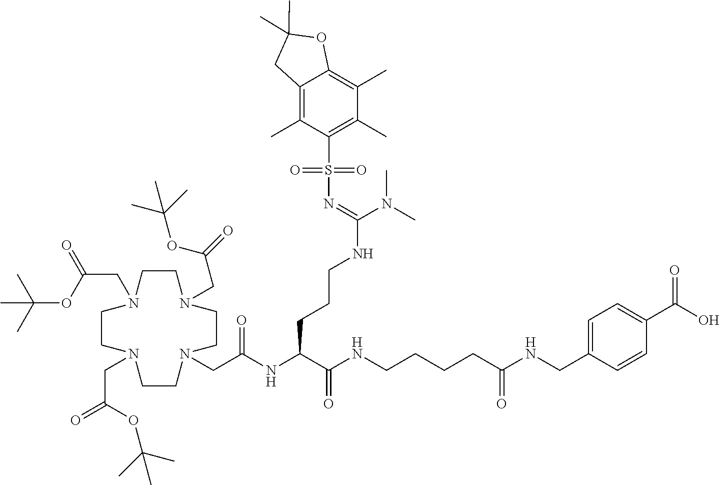 [SMILES: Cc1c(C)c(S(=O)(=O)/N=C(/NCCC[C@H](NC(=O)CN2CCN(CC(=O)OC(C)(C)C)CCN(CC(=O)OC(C)(C)C)CCN(CC(=O)OC(C)(C)C)CC2)C(=O)NCCCCC(=O)NCc2ccc(C(=O)O)cc2)N(C)C)c(C)c2c1OC(C)(C)C2]

Initial resin loading with 4-(((((9H-fluoren-9-yl)methoxy)carbonyl)amino)methyl)benzoic acid was performed as described in SP-1 (working on a 0.1 mmol scale). The Fmoc group was removed according to SP-3 and was coupled with 5-((((9H-fluoren-9-yl)methoxy)carbonyl)amino)pentanoic acid according to SP-2. The Fmoc group was removed according to SP-3 and then was coupled with (S,Z)-2-((((9H-fluoren-9-yl)methoxy)carbonyl)amino)-5-(((dimethylamino)((2,2,4,6,7-pentamethyl-2,3-dihydrobenzofuran)-5-sulfonamido)methylene)amino)pentanoic acid according to SP-2. The Fmoc group was removed according to SP-3 and then coupled with 2-(4,7,10-tris(2-(tert-butoxy)-2-oxoethyl)-1,4,7,10-tetraazacyclododecan-1-yl)acetic acid according to SP-2. The compound was cleaved from the resin according to SP-4 to give the title compound (0.04 g, 32%). ESI/MS m/z: 1242 (M+H).

### Preparation 43

### 4-((8S,14S,E)-8-(2-(7-(2-Amino-2-oxoethyl)-4,10-bis(2-(tert-butoxy)-2-oxoethyl)-1,4,7,10-tetraazacyclododecan-1-yl)acetamido)-14-(2-(tert-butoxy)-2-oxoethyl)-2-methyl-9,12,15-trioxo-3-((2,2,4,5,7-pentamethyl-2,3-dihydrobenzofuran)-6-sulfonamido)-2,4,10,13,16-pentaazaheptadec-3-en-17-yl)benzoic acid

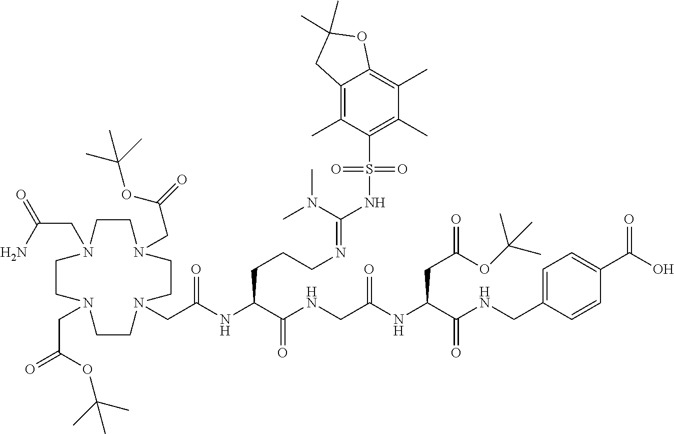 [SMILES: Cc1c(C)c(S(=O)(=O)N/C(=N/CCC[C@H](NC(=O)CN2CCN(CC(=O)OC(C)(C)C)CCN(CC(N)=O)CCN(CC(=O)OC(C)(C)C)CC2)C(=O)NCC(=O)N[C@@H](CC(=O)OC(C)(C)C)C(=O)NCc2ccc(C(=O)O)cc2)N(C)C)c(C)c2c1OC(C)(C)C2]

The initial resin loading with 4-(((((9H-fluoren-9-yl)methoxy)carbonyl)amino)methyl)benzoic acid was performed as described in SP-1 (working on a 0.1 mol scale). The Fmoc group was removed according to SP-3 and then coupled with (S)-2-((((9H-fluoren-9-yl)methoxy)carbonyl)amino)-4-(tert-butoxy)-4-oxobutanoic acid according to SP-2. The Fmoc group was removed according to SP-3 and then coupled with (((9H-fluoren-9-yl)methoxy)carbonyl)glycine according to SP-2. The Fmoc group was removed according to SP-3 and then coupled with (S,Z)-2-((((9H-fluoren-9-yl)methoxy)carbonyl)amino)-5-(((dimethylamino)((2,2,4,6,7-pentamethyl-2,3-dihydrobenzofuran)-5-sulfonamido)methylene)amino)pentanoic acid according to SP-2 but using PyBroP as the coupling reagent. The amino group was removed according to SP-3 and then coupled with 2-(7-(2-amino-2-oxoethyl)-4,10-bis(2-(tert-butoxy)-2-oxoethyl)-1,4,7,10-tetraazacyclododecan-1-yl)acetic acid according to SP-2. The compound was cleaved from the resin according to SP-4 to give the title compound (0.04 g, 26%). ESI/MS m/z: 1314 (M+H).

### Preparation 44

### 4-((8S,14S,E)-14-(2-(tert-Butoxy)-2-oxoethyl)-2-methyl-9,12,15-trioxo-3-((2,2,4,5,7-pentamethyl-2,3-dihydrobenzofuran)-6-sulfonamido)-8-(2-(4,7,10-tris(2-(tert-butoxy)-2-oxoethyl)-1,4,7,10-tetraazacyclododecan-1-yl)acetamido)-2,4,10,13,16-pentaazaheptadec-3-en-17-yl)benzoic acid

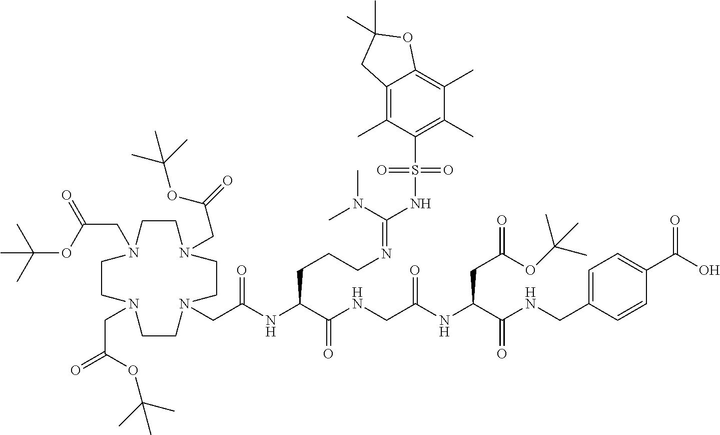 [SMILES: Cc1c(C)c(S(=O)(=O)N/C(=N/CCC[C@H](NC(=O)CN2CCN(CC(=O)OC(C)(C)C)CCN(CC(=O)OC(C)(C)C)CCN(CC(=O)OC(C)(C)C)CC2)C(=O)NCC(=O)N[C@@H](CC(=O)OC(C)(C)C)C(=O)NCc2ccc(C(=O)O)cc2)N(C)C)c(C)c2c1OC(C)(C)C2]

The initial resin loading with 4-(((((9H-fluoren-9-yl)methoxy)carbonyl)amino)methyl)benzoic acid was performed as described in SP-1 (working on a 0.1 mmol scale). The Fmoc group was removed according to SP-3 and then coupled with (S)-2-((((9H-fluoren-9-yl)methoxy)carbonyl)amino)-4-(tert-butoxy)-4-oxobutanoic acid according to SP-2. The Fmoc group was removed according to SP-3 and then coupled with (((9H-fluoren-9-yl)methoxy)carbonyl)glycine according to SP-2. The Fmoc group was removed according to SP-3 and then coupled with N²-(((9H-fluoren-9-yl)methoxy)carbonyl)-N^{w},N^{w}-dimethyl-L-arginine according to SP-2 but using PyBroP as the coupling reagent. The Fmoc group was removed according to SP-3 and then coupled with 2-(4,7,10-tris(2-(tert-butoxy)-2-oxoethyl)-1,4,7,10-tetraazacyclododecan-1-yl)acetic acid according to SP-2. The compound was cleaved from the resin according to SP-4 to give the title compound (0.03 g, 22%). ESI/MS m/z: 1371 (M+H).

### Preparation 45

### (1SR,4SR)-4-((S,E)-8-(2-(7-(2-Amino-2-oxoethyl)-4,10-bis(2-(tert-butoxy)-2-oxoethyl)-1,4,7,10-tetraazacyclododecan-1-yl)acetamido)-2,10-dimethyl-9,15-dioxo-3-((2,2,4,6,7-pentamethyl-2,3-dihydrobenzofuran)-5-sulfonamido)-2,4,10,16-tetraazaheptadec-3-en-17-yl)cyclohexane-1-carboxylic acid

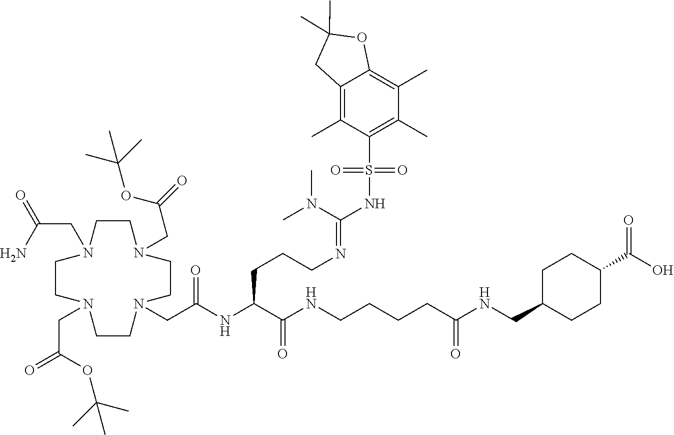 [SMILES: Cc1c(C)c(S(=O)(=O)N/C(=N/CCC[C@H](NC(=O)CN2CCN(CC(=O)OC(C)(C)C)CCN(CC(N)=O)CCN(CC(=O)OC(C)(C)C)CC2)C(=O)NCCCCC(=O)NC[C@H]2CC[C@H](C(=O)O)CC2)N(C)C)c(C)c2c1OC(C)(C)C2]

The initial resin loading with (1r,4r)-4-(aminomethyl)cyclohexane-1-carboxylic acid was performed as described in SP-1 (working on a 0.1 mmol scale). The Fmoc group was removed according to SP-3 and then coupled with 5-((((9H-fluoren-9-yl)methoxy)carbonyl)(methyl)amino)pentanoic acid according to SP-2 using PyBroP as the coupling reagent. The Fmoc group was removed according to SP-3 and then coupled with (S,E)-2-((((9H-fluoren-9-yl)methoxy)carbonyl)amino)-5-(((dimethylamino)((2,2,4,6,7-pentamethyl-2,3-dihydrobenzofuran)-5-sulfonamido)methylene)amino)pentanoic acid according to SP-2 using PyBroP as the coupling reagent. The amino group was deprotected according to SP-3 and then coupled with 2-(7-(2-amino-2-oxoethyl)-4,10-bis(2-(tert-butoxy)-2-oxoethyl)-1,4,7,10-tetraazacyclododecan-1-yl)acetic acid according to SP-2. The compound was cleaved from the resin according to SP-4 to give the title compound (0.04 g, 33%). ESI/MS m/z: 1205 (M+H).

### Preparation 46

### (1SR,4SR)-4-((S,Z)-2,10-Dimethyl-9,15-dioxo-3-((2,2,4,6,7-pentamethyl-2,3-dihydrobenzofuran)-5-sulfonamido)-8-(2-(4,7,10-tris(2-(tert-butoxy)-2-oxoethyl)-1,4,7,10-tetraazacyclododecan-1-yl)acetamido)-2,4,10,16-tetraazaheptadec-3-en-17-yl)cyclohexane-1-carboxylic acid

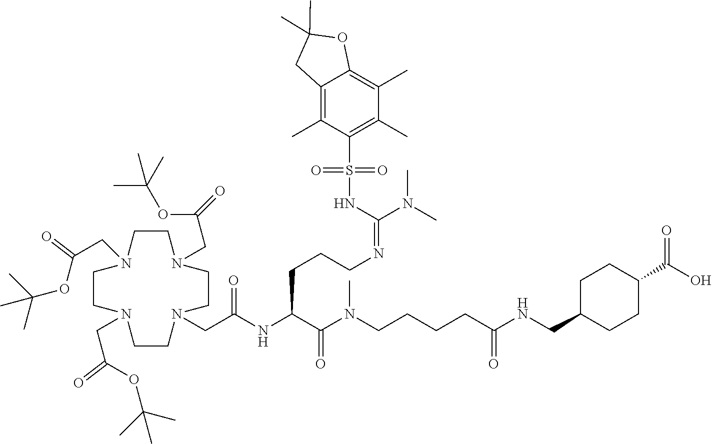 [SMILES: Cc1c(C)c(S(=O)(=O)N/C(=N\CCC[C@H](NC(=O)CN2CCN(CC(=O)OC(C)(C)C)CCN(CC(=O)OC(C)(C)C)CCN(CC(=O)OC(C)(C)C)CC2)C(=O)N(C)CCCCC(=O)NC[C@H]2CC[C@H](C(=O)O)CC2)N(C)C)c(C)c2c1OC(C)(C)C2]

The initial resin loading with (1r,4r)-4-(aminomethyl)cyclohexane-1-carboxylic acid was performed as described in SP-1 (working on a 0.1 mmol scale). The Fmoc group was removed according to SP-3 and then coupled with 5-((((9H-fluoren-9-yl)methoxy)carbonyl)(methyl)amino)pentanoic acid according to SP-2 using PyBroP as the coupling reagent. The Fmoc group was removed according to SP-3 and then coupled with (S,E)-2-((((9H-fluoren-9-yl)methoxy)carbonyl)amino)-5-(((dimethylamino)((2,2,4,6,7-pentamethyl-2,3-dihydrobenzofuran)-5-sulfonamido)methylene)amino)pentanoic acid according to SP-2 using PyBroP as the coupling reagent. The amino group was deprotected according to SP-3 and then coupled with 2-(7-(2-amino-2-oxoethyl)-4,10-bis(2-(tert-butoxy)-2-oxoethyl)-1,4,7,10-tetraazacyclododecan-1-yl)acetic acid according to SP-2. The compound was cleaved from the resin according to SP-4 to give the title compound (0.04 g, 31%). ESI/MS m/z: 1263 (M+H).

### Preparation 47

### N¹-(5-((S)-2-Amino-5-(3-((2,2-dimethyl-2,3-dihydrobenzofuran-5-yl)sulfonyl)guanidino)pentanamido)pentyl)-N⁴-((2R)-1-((2R,4S)-4-hydroxy-2-((3aS,4S,6S)-3a,5,5-trimethylhexahydro-4,6-methanobenzo[d][1,3,2]dioxaborol-2-yl)pyrrolidin-1-yl)-1-oxopropan-2-yl)naphthalene-1,4-dicarboxamide

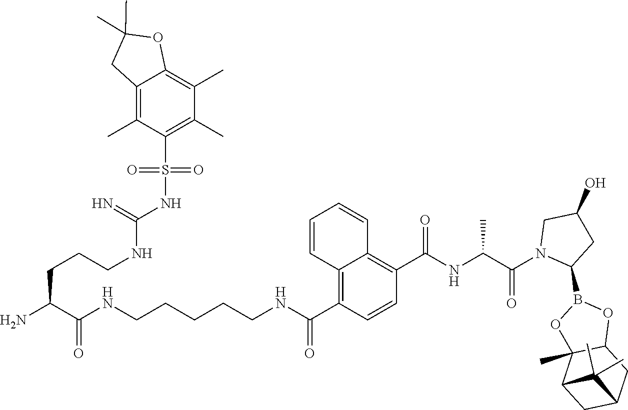

(S)-4-((5-(2-((((9H-Fluoren-9-yl)methoxy)carbonyl)amino)-5-(3-((2,2,4,6,7-pentamethyl-2,3-dihydrobenzofuran-5-yl)sulfonyl)guanidino)pentanamido)pentyl)carbamoyl)-1-naphthoic acid was coupled with (2R)-2-amino-1-((2R,4S)-4-hydroxy-2-((3aS,4S,6S)-3a,5,5-trimethylhexahydro-4,6-methanobenzo[d][1,3,2]dioxaborol-2-yl)pyrrolidin-1-yl)propan-1-one HCl according to SP-5 (working on a 0.15 mmol scale). The Fmoc group was removed according to SP-3. The crude product was further purified using the General Procedure for Preparative HPLC purification (eluent gradient: 20% B for the first 3 min, then from 20% to 98% B over 12 min, which was maintained for the next 5 min). Both peaks of the desired compound and de-pinanediol compound were collected, combined, concentrated under reduced pressure, and lyophilized to give compound the title compound (0.10 g, 58%). ESI/MS m/z: 894 (M-pinanediol+H).

### Preparation 48

### (S)-4-((5-(2-(2-(7-(2-Amino-2-oxoethyl)-4,10-bis(2-(tert-butoxy)-2-oxoethyl)-1,4,7,10-tetraazacyclododecan-1-yl)acetamido)-5-(3-((2,2,4,5,7-pentamethyl-2,3-dihydrobenzofuran-6-yl)sulfonyl)guanidino)pentanamido)pentyl)carbamoyl)-1-naphthoic acid

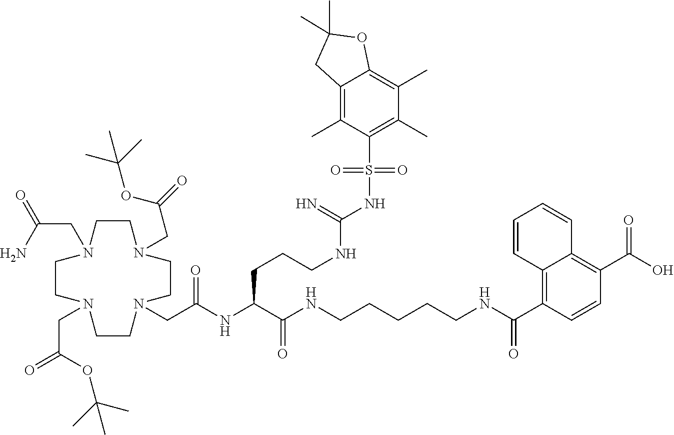 [SMILES: Cc1c(C)c(S(=O)(=O)NC(=N)NCCC[C@H](NC(=O)CN2CCN(CC(=O)OC(C)(C)C)CCN(CC(N)=O)CCN(CC(=O)OC(C)(C)C)CC2)C(=O)NCCCCCNC(=O)c2ccc(C(=O)O)c3ccccc23)c(C)c2c1OC(C)(C)C2]

The initial resin loading with naphthalene-1,4-dicarboxylic acid was performed according to SP-1 (working on a 0.1 mmol scale) and then coupled with mono-Fmoc-1,5-diaminopentane HCl according to SP-2. The Fmoc group was removed according to SP-3 and then coupled with N²-(((9H-fluoren-9-yl)methoxy)carbonyl)-N^{w}-((2,2,4,6,7-pentamethyl-2,3-dihydrobenzofuran-5-yl)sulfonyl)-L-arginine according to SP-2 using PyBroP as the coupling reagent. The Fmoc group was removed according to SP-3 and then coupled with 2-(7-(2-amino-2-oxoethyl)-4,10-bis(2-(tert-butoxy)-2-oxoethyl)-1,4,7,10-tetraazacyclododecan-1-yl)acetic acid according to SP-2. The compound was cleaved from the resin according to SP-4 to give the title compound (0.04 g, 29%). ESI/MS m/z: 1207 (M).

### Preparation 49

### 4-((5-((S)-5-(((Z)-(Dimethylamino)((2,2,4,6,7-pentamethyl-2,3-dihydrobenzofuran)-5-sulfonamido)methylene)amino)-2-((S)-2-hexanamido-6-(2-(4,7,10-tris(2-(tert-butoxy)-2-oxoethyl)-1,4,7,10-tetraazacyclododecan-1-yl)acetamido)hexanamido)pentanamido)pentyl)carbamoyl)-1-naphthoic acid

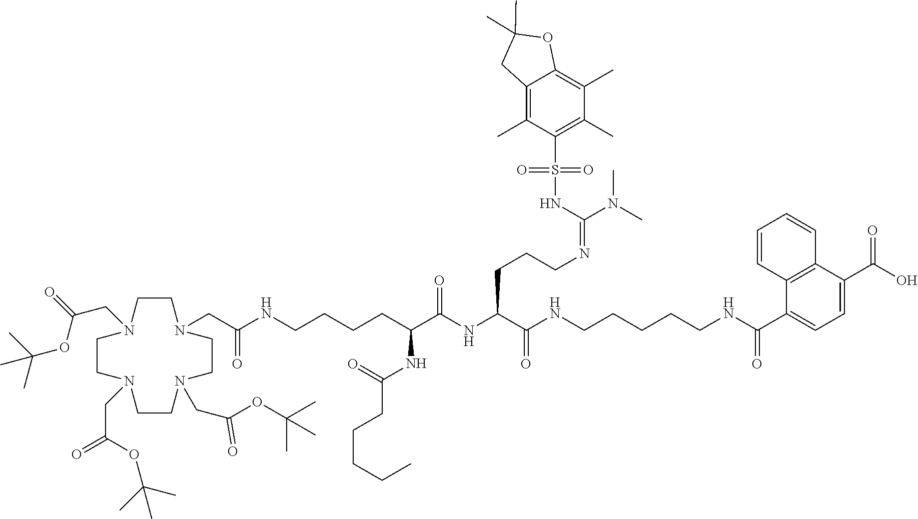 [SMILES: CCCCCC(=O)N[C@@H](CCCCNC(=O)CN1CCN(CC(=O)OC(C)(C)C)CCN(CC(=O)OC(C)(C)C)CCN(CC(=O)OC(C)(C)C)CC1)C(=O)N[C@@H](CCC/N=C(\NS(=O)(=O)c1c(C)c(C)c2c(c1C)CC(C)(C)O2)N(C)C)C(=O)NCCCCCNC(=O)c1ccc(C(=O)O)c2ccccc12]

The initial resin loading with naphthalene-1,4-dicarboxylic acid was performed as described in SP-1 (working on a 0.1 mmol scale) and then coupled with (9H-fluoren-9-yl)methyl (5-aminopentyl)carbamate HCl according to SP-2 with PyBroP instead of HATU as the coupling reagent. The Fmoc group was removed according to SP-3 and then coupled with (S,Z)-2-((((9H-fluoren-9-yl)methoxy)carbonyl)amino)-5-(((dimethylamino)((2,2,4,6,7-pentamethyl-2,3-dihydrobenzofuran)-5-sulfonamido)methylene)amino)pentanoic acid according to SP-2. The Fmoc group was removed according to SP-3 and then coupled to N²-(((9H-fluoren-9-yl)methoxy)carbonyl)-N⁶-(1-(4,4-dimethyl-2,6-dioxocyclohexylidene)ethyl)-L-lysine according to SP-2. The Fmoc group was removed according to SP-3 and then acylated with hexanoyl chloride and DIPEA in DCM. The -Dde protection was removed using 1% hydrazine hydrate in DMF and then coupled with 2-(4,7,10-tris(2-(tert-butoxy)-2-oxoethyl)-1,4,7,10-tetraazacyclododecan-1-yl)acetic acid according to SP-2 using PyBroP instead of HATU as the coupling reagent. The compound was cleaved from the resin according to SP-4 to give the title compound (0.03 g, 20%). ESI/MS m/z: 1519 (M+H).

### Preparation 50

### (S,Z)-4-((18-(3-(3,3-Dimethyl-2-((2,2,4,6,7-pentamethyl-2,3-dihydrobenzofuran-5-yl)sulfonyl)guanidino)propyl)-2,10,16,19-tetraoxo-1-(4,7,10-tris(2-(tert-butoxy)-2-oxoethyl)-1,4,7,10-tetraazacyclododecan-1-yl)-3,9,17,20-tetraazapentacosan-25-yl)carbamoyl)-1-naphthoic acid

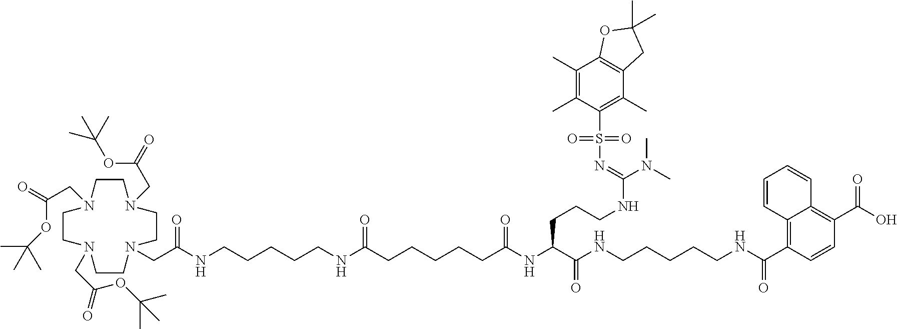 [SMILES: Cc1c(C)c(S(=O)(=O)/N=C(/NCCC[C@H](NC(=O)CCCCCC(=O)NCCCCCNC(=O)CN2CCN(CC(=O)OC(C)(C)C)CCN(CC(=O)OC(C)(C)C)CCN(CC(=O)OC(C)(C)C)CC2)C(=O)NCCCCCNC(=O)c2ccc(C(=O)O)c3ccccc23)N(C)C)c(C)c2c1OC(C)(C)C2]

The initial resin loading with naphthalene-1,4-dicarboxylic acid was performed as described in SP-1 (working on a 0.1 mmol scale) and then coupled with (9H-fluoren-9-yl)methyl (5-aminopentyl)carbamate HCl according to SP-2 with PyBroP instead of HATU as the coupling reagent. The Fmoc group was removed according to SP-3 and then coupled with (S,Z)-2-((((9H-fluoren-9-yl)methoxy)carbonyl)amino)-5-(((dimethylamino)((2,2,4,6,7-pentamethyl-2,3-dihydrobenzofuran)-5-sulfonamido)methylene)amino)pentanoic acid according to SP-2. The Fmoc group was removed according to SP-3 and then coupled with 7-(4-nitrophenoxy)-7-oxoheptanoic acid in DMF with DIPEA. The free carboxylic acid was then coupled to (9H-fluoren-9-yl)methyl (5-aminopentyl)carbamate HCl according to SP-2 with PyBroP instead of HATU. The Fmoc group was removed according to SP-3 and then coupled with 2-(4,7,10-tris(2-(tert-butoxy)-2-oxoethyl)-1,4,7,10-tetraazacyclododecan-1-yl)acetic acid according to SP-2 using PyBroP instead of HATU as the coupling reagent. The compound was cleaved from the resin according to SP-4 to give the title compound (0.035 g, 20%). ESI/MS m/z: 1519 (M+H).

### Preparation 51

### 4-((5-((S)-5-(((Z)-(Dimethylamino)((2,2,4,6,7-pentamethyl-2,3-dihydrobenzofuran)-5-sulfonamido)methylene)amino)-2-((S)-2-(4-((4-(4-iodophenyl)butanamido)methyl)benzamido)-6-(2-(4,7,10-tris(2-(tert-butoxy)-2-oxoethyl)-1,4,7,10-tetraazacyclododecan-1-yl)acetamido)hexanamido)pentanamido)pentyl)carbamoyl)-1-naphthoic acid

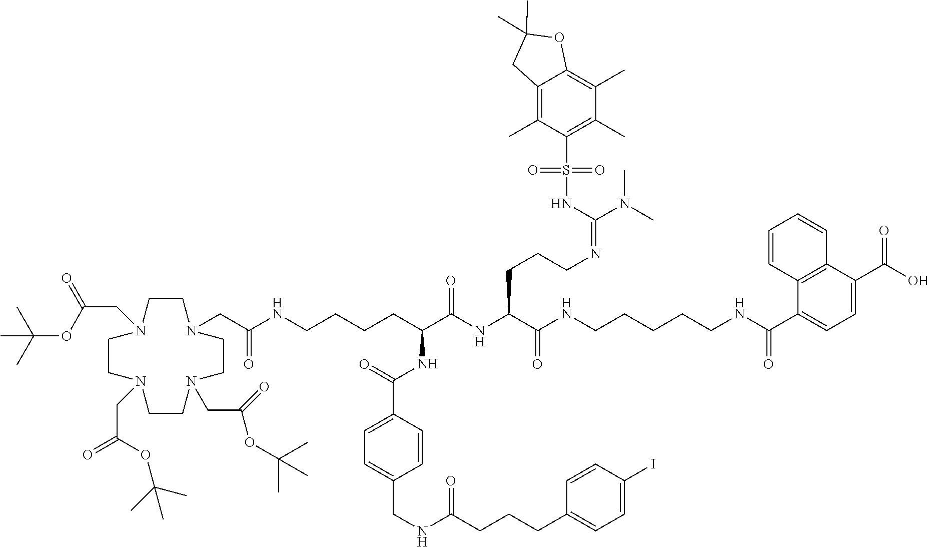 [SMILES: Cc1c(C)c(S(=O)(=O)N/C(=N\CCC[C@H](NC(=O)[C@H](CCCCNC(=O)CN2CCN(CC(=O)OC(C)(C)C)CCN(CC(=O)OC(C)(C)C)CCN(CC(=O)OC(C)(C)C)CC2)NC(=O)c2ccc(CNC(=O)CCCc3ccc(I)cc3)cc2)C(=O)NCCCCCNC(=O)c2ccc(C(=O)O)c3ccccc23)N(C)C)c(C)c2c1OC(C)(C)C2]

The initial resin loading with naphthalene-1,4-dicarboxylic acid was performed as described in SP-1 (working on a 0.1 mmol scale) and then coupled with (9H-fluoren-9-yl)methyl (5-aminopentyl)carbamate HCl according to SP-2 with PyBroP instead of HATU as the coupling reagent. The Fmoc group was removed according to SP-3 and then coupled with (S,Z)-2-((((9H-fluoren-9-yl)methoxy)carbonyl)amino)-5-(((dimethylamino)((2,2,4,6,7-pentamethyl-2,3-dihydrobenzofuran)-5-sulfonamido)methylene)amino)pentanoic acid according to SP-2. The Fmoc group was removed according to SP-3 and then coupled to N²-(((9H-fluoren-9-yl)methoxy)carbonyl)-N⁶-(1-(4,4-dimethyl-2,6-dioxocyclohexylidene)ethyl)-L-lysine according to SP-2. The Fmoc group was removed according to SP-3 and then coupled to 4-(((((9H-fluoren-9-yl)methoxy)carbonyl)amino)methyl)benzoic acid according to SP-2 with PyBroP instead of HATU as the coupling reagent. The Fmoc group was removed according to SP-3 and then coupled to 4-iodophenylbutanoic acid according to SP-2 with PyBroP instead of HATU as the coupling reagent. The -Dde protection was removed with 2% hydrazine hydrate in DMF and then coupled with 2-(4,7,10-tris(2-(tert-butoxy)-2-oxoethyl)-1,4,7,10-tetraazacyclododecan-1-yl)acetic acid according to SP-2 with PyBroP instead of HATU as the coupling reagent. The compound was cleaved from the resin according to SP-4 to give the title compound (0.03 g, 16%). ESI/MS m/z: 1826 (M+H).

### Preparation 52

### 4-((5-((S)-5-(((Z)-(Dimethylamino)((2,2,4,6,7-pentamethyl-2,3-dihydrobenzofuran)-5-sulfonamido)methylene)amino)-2-((S)-2-(4-(4-iodophenyl)butanamido)-6-(2-(4,7,10-tris(2-(tert-butoxy)-2-oxoethyl)-1,4,7,10-tetraazacyclododecan-1-yl)acetamido)hexanamido)pentanamido)pentyl)carbamoyl)-1-naphthoic acid

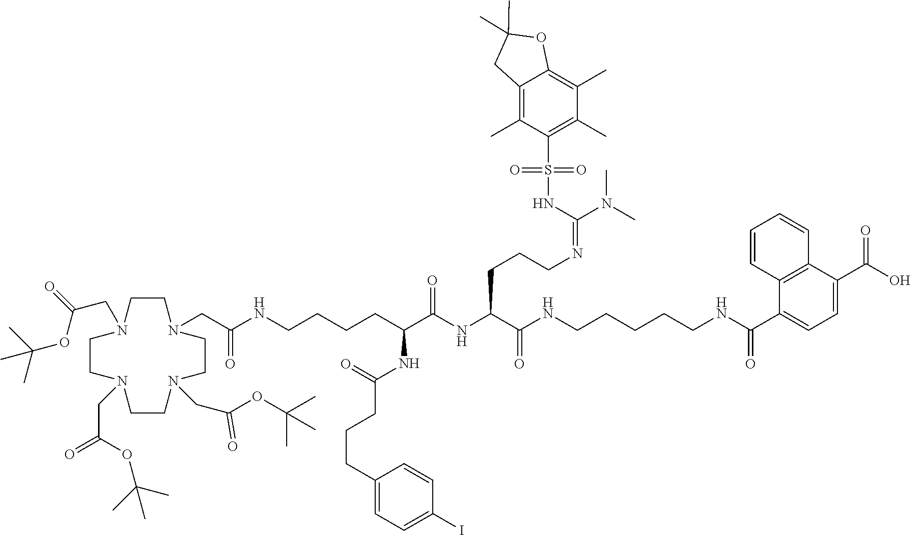 [SMILES: Cc1c(C)c(S(=O)(=O)N/C(=N\CCC[C@H](NC(=O)[C@H](CCCCNC(=O)CN2CCN(CC(=O)OC(C)(C)C)CCN(CC(=O)OC(C)(C)C)CCN(CC(=O)OC(C)(C)C)CC2)NC(=O)CCCc2ccc(I)cc2)C(=O)NCCCCCNC(=O)c2ccc(C(=O)O)c3ccccc23)N(C)C)c(C)c2c1OC(C)(C)C2]

The initial resin loading with naphthalene-1,4-dicarboxylic acid was performed as described in SP-1 (working on a 0.1 mmol scale) and then coupled with (9H-fluoren-9-yl)methyl (5-aminopentyl)carbamate HCl according to SP-2 with PyBroP instead of HATU as the coupling reagent. The Fmoc group was removed according to SP-3 and then coupled with (S,Z)-2-((((9H-fluoren-9-yl)methoxy)carbonyl)amino)-5-(((dimethylamino)((2,2,4,6,7-pentamethyl-2,3-dihydrobenzofuran)-5-sulfonamido)methylene)amino)pentanoic acid according to SP-2. The Fmoc group was removed according to SP-3 and then coupled to N²-(((9H-fluoren-9-yl)methoxy)carbonyl)-N⁶-(1-(4,4-dimethyl-2,6-dioxocyclohexylidene)ethyl)-L-lysine according to SP-2. The Fmoc group was removed according to SP-3 and then coupled with 4-(4-iodophenyl)butyric acid according to SP-2 with PyBroP instead of HATU as the coupling reagent. The -Dde protecting group was removed with 1% hydrazine hydrate in DMF and then coupled with 2-(4,7,10-tris(2-(tert-butoxy)-2-oxoethyl)-1,4,7,10-tetraazacyclododecan-1-yl)acetic acid according to SP-2 using PyBroP instead of HATU as the coupling reagent. The compound was cleaved from the resin according to SP-4 to give the title compound (0.04 g, 20%). ESI/MS m/z: 1693 (M+H).

### Preparation 53

### 4-(((6R,9S,12S)-12-(3-(((Z)-(Dimethylamino)((2,2,4,6,7-pentamethyl-2,3-dihydrobenzofuran)-5-sulfonamido)methylene)amino)propyl)-6-(4-(4-iodophenyl)butanamido)-2,2-dimethyl-4,7,10,13-tetraoxo-9-(4-(2-(4,7,10-tris(2-(tert-butoxy)-2-oxoethyl)-1,4,7,10-tetraazacyclododecan-1-yl)acetamido)butyl)-3-oxa-8,11,14-triazanonadecan-19-yl)carbamoyl)-1-naphthoic acid

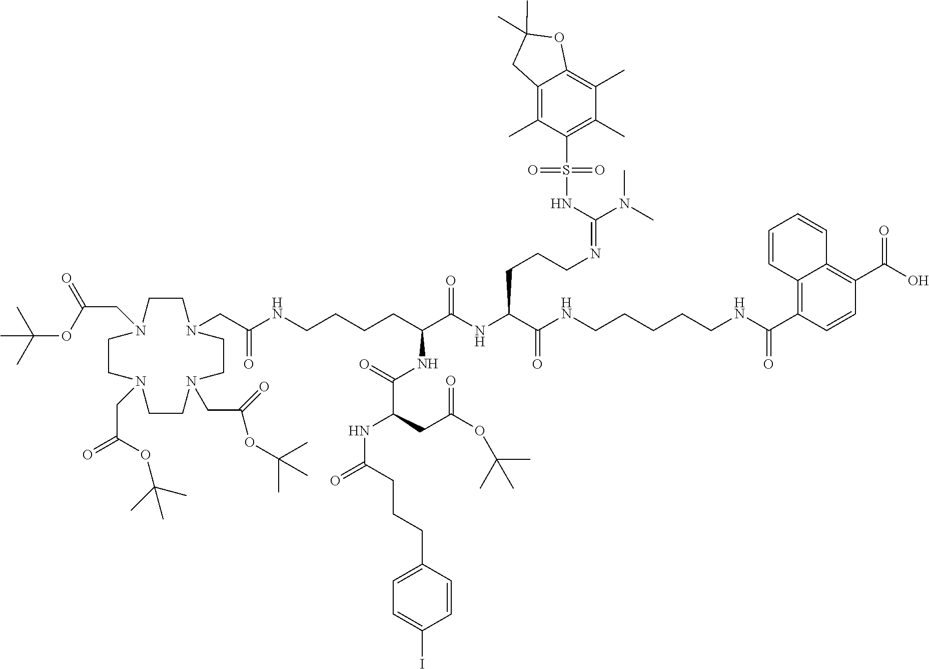 [SMILES: Cc1c(C)c(S(=O)(=O)N/C(=N\CCC[C@H](NC(=O)[C@H](CCCCNC(=O)CN2CCN(CC(=O)OC(C)(C)C)CCN(CC(=O)OC(C)(C)C)CCN(CC(=O)OC(C)(C)C)CC2)NC(=O)[C@@H](CC(=O)OC(C)(C)C)NC(=O)CCCc2ccc(I)cc2)C(=O)NCCCCCNC(=O)c2ccc(C(=O)O)c3ccccc23)N(C)C)c(C)c2c1OC(C)(C)C2]

The initial resin loading with naphthalene-1,4-dicarboxylic acid was performed as described in SP-1 (working on a 0.1 mmol scale) and then coupled with (9H-fluoren-9-yl)methyl (5-aminopentyl)carbamate HCl according to SP-2 with PyBroP instead of HATU as the coupling reagent. The Fmoc group was removed according to SP-3 and then coupled with (S,Z)-2-((((9H-fluoren-9-yl)methoxy)carbonyl)amino)-5-(((dimethylamino)((2,2,4,6,7-pentamethyl-2,3-dihydrobenzofuran)-5-sulfonamido)methylene)amino)pentanoic acid according to SP-2. The Fmoc group was removed according to SP-3 and then coupled to N²-(((9H-fluoren-9-yl)methoxy)carbonyl)-N⁶-(1-(4,4-dimethyl-2,6-dioxocyclohexylidene)ethyl)-L-lysine according to SP-2. The Fmoc group was removed according to SP-3 and then coupled to (R)-2-((((9H-fluoren-9-yl)methoxy)carbonyl)amino)-4-(tert-butoxy)-4-oxobutanoic acid according to SP-2 with PyBroP instead of HATU as the coupling reagent. The Fmoc group was removed according to SP-3 and then coupled to 4-iodophenylbutanoic acid according to SP-2 with PyBroP instead of HATU as the coupling reagent. The -Dde protection was removed using 2% hydrazine hydrate in DMF and then conjugated with 2-(4,7,10-tris(2-(tert-butoxy)-2-oxoethyl)-1,4,7,10-tetraazacyclododecan-1-yl)acetic acid according to SP-2 with PyBroP instead of HATU as the coupling reagent. The compound was cleaved from the resin according to SP-4 to give the title compound (0.03 g, 16%). ESI/MS m/z: 1864 (M+H).

### Preparation 54

### (1S,4r)-4-((S,Z)-2,10-Dimethyl-9,15-dioxo-3-((2,2,4,6,7-pentamethyl-2,3-dihydrobenzofuran)-5-sulfonamido)-8-(2-(4,7,10-tris(2-(tert-butoxy)-2-oxoethyl)-1,4,7,10-tetraazacyclododecan-1-yl)acetamido)-2,4,10,16-tetraazaheptadec-3-en-17-yl)cyclohexane-1-carboxylic acid

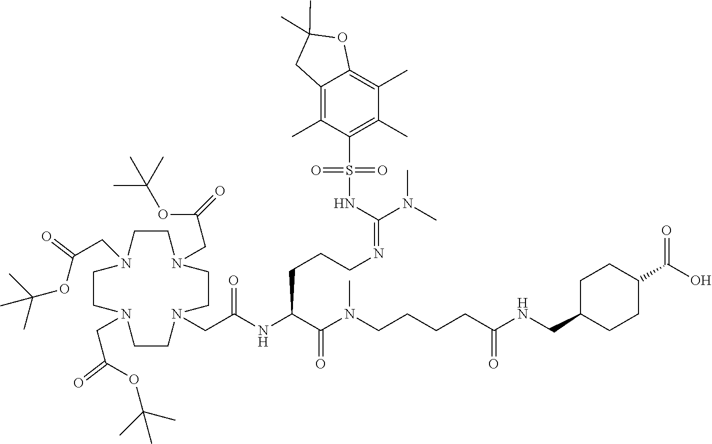

The initial resin loading with (1r,4r)-4-(aminomethyl)cyclohexane-1-carboxylic acid was performed as described in SP-1 (working on a 0.1 mmol scale). The Fmoc group was removed according to SP-3 and then coupled with 5-((((9H-fluoren-9-yl)methoxy)carbonyl)(methyl)amino)pentanoic acid according to SP-2 using PyBroP instead of HATU as the coupling reagent. The Fmoc group was removed according to SP-3 and then coupled with (S,Z)-2-((((9H-fluoren-9-yl)methoxy)carbonyl)amino)-5-(((dimethylamino)((2,2,4,6,7-pentamethyl-2,3-dihydrobenzofuran)-5-sulfonamido)methylene)amino)pentanoic acid according to SP-2 but using PyBroP as the coupling reagent. The Fmoc group was removed according to SP-3 and then coupled with 2-(4,7,10-tris(2-(tert-butoxy)-2-oxoethyl)-1,4,7,10-tetraazacyclododecan-1-yl)acetic acid according to SP-2 using PyBroP instead of HATU as the coupling reagent. The compound was cleaved from the resin according to SP-4 to give the title compound (0.05 g, 35%). ESI/MS m/z: 1263 (M+H).

### Preparation 55

### 2,2′-(4-(2-Amino-2-oxoethyl)-10-(2-(((S)-1-((5-(4-(((R)-1-((R)-2-boronopyrrolidin-1-yl)-1-oxopropan-2-yl)carbamoyl)-1-naphthamido)pentyl)amino)-5-guanidino-1-oxopentan-2-yl)amino)-2-oxoethyl)-1,4,7,10-tetraazacyclododecane-1,7-diyl)diacetic acid

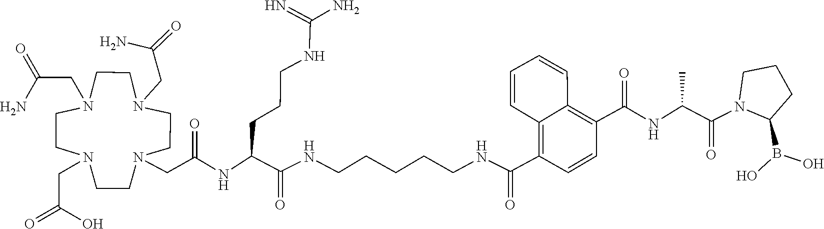 [SMILES: C[C@@H](NC(=O)c1ccc(C(=O)NCCCCCNC(=O)[C@H](CCCNC(=N)N)NC(=O)CN2CCN(CC(N)=O)CCN(CC(N)=O)CCN(CC(=O)O)CC2)c2ccccc12)C(=O)N1CCC[C@H]1B(O)O]

(S)-4-((5-(2-(2-(7-(2-Amino-2-oxoethyl)-4,10-bis(2-(tert-butoxy)-2-oxoethyl)-1,4,7,10-tetraazacyclododecan-1-yl)acetamido)-5-(3-((2,2,4,5,7-pentamethyl-2,3-dihydrobenzofuran-6-yl)sulfonyl)guanidino)pentanamido)pentyl)carbamoyl)-1-naphthoic acid was coupled with ((R)-1-(D-alanyl)pyrrolidin-2-yl)boronic acid HCl according to SP-5 and the protecting groups (—Pbf, -tBu, and pinanediol) removed according to SP-6 to afford the title compound (5.1 mg, 38% yield at 0.02 mmol scale) as a white powder. ESI/MS m/z: 993 (M−H₂O+H).

### Preparation 56

### 2,2′-(4-(2-Amino-2-oxoethyl)-10-(2-(((S)-1-((5-(4-(((R)-1-((2R,4S)-2-borono-4-hydroxypyrrolidin-1-yl)-1-oxopropan-2-yl)carbamoyl)-1-naphthamido)pentyl)amino)-5-guanidino-1-oxopentan-2-yl)amino)-2-oxoethyl)-1,4,7,10-tetraazacyclododecane-1,7-diyl)diacetic acid

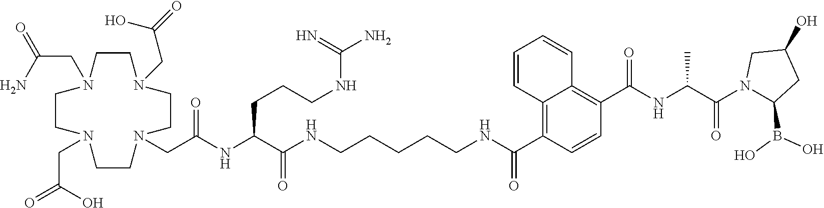 [SMILES: C[C@@H](NC(=O)c1ccc(C(=O)NCCCCCNC(=O)[C@H](CCCNC(=N)N)NC(=O)CN2CCN(CC(=O)O)CCN(CC(N)=O)CCN(CC(=O)O)CC2)c2ccccc12)C(=O)N1C[C@@H](O)C[C@H]1B(O)O]

N¹-(5-((S)-2-Amino-5-(3-((2,2-dimethyl-2,3-dihydrobenzofuran-5-yl)sulfonyl)guanidino)pentanamido)pentyl)-N4-((2R)-1-((2R,4S)-4-hydroxy-2-((3aS,4S,6S)-3a,5,5-trimethylhexahydro-4,6-methanobenzo[d][1,3,2]dioxaborol-2-yl)pyrrolidin-1-yl)-1-oxopropan-2-yl)naphthalene-1,4-dicarboxamide was coupled with 2-(7-(2-amino-2-oxoethyl)-4,10-bis(2-(tert-butoxy)-2-oxoethyl)-1,4,7,10-tetraazacyclododecan-1-yl)acetic acid according to SP-2 and the protecting groups (—Pbf, -tBu, and pinanediol) removed according to SP-6 to afford the title compound (6.7 mg, 25% yield at 0.02 mmol scale) as a white powder. ESI/MS m/z: 1009 (M−H₂O+H).

### Preparation 57

### 2,2′-(4-(2-Amino-2-oxoethyl)-10-(2-(((S)-1-((5-((4-(((R)-1-((2R,4S)-2-borono-4-hydroxypyrrolidin-1-yl)-1-oxopropan-2-yl)carbamoyl)benzyl)(methyl)amino)-5-oxopentyl)amino)-5-guanidino-1-oxopentan-2-yl)amino)-2-oxoethyl)-1,4,7,10-tetraazacyclododecane-1,7-diyl)diacetic acid

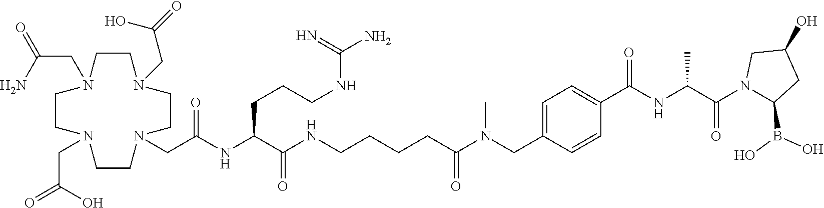 [SMILES: C[C@@H](NC(=O)c1ccc(CN(C)C(=O)CCCCNC(=O)[C@H](CCCNC(=N)N)NC(=O)CN2CCN(CC(=O)O)CCN(CC(N)=O)CCN(CC(=O)O)CC2)cc1)C(=O)N1C[C@@H](O)C[C@H]1B(O)O]

(S)-4-((5-(2-(2-(7-(2-Amino-2-oxoethyl)-4,10-bis(2-(tert-butoxy)-2-oxoethyl)-1,4,7,10-tetraazacyclododecan-1-yl)acetamido)-5-(3-((2,2,4,5,7-pentamethyl-2,3-dihydrobenzofuran-6-yl)sulfonyl)guanidino)pentanamido)-N-methylpentanamido)methyl)benzoic acid was coupled with ((2R,4S)-1-(D-alanyl)-4-hydroxypyrrolidin-2-yl)boronic acid HCl according to SP-5 and the protecting groups (—Pbf, -tBu, and pinanediol) removed according to SP-6 to afford the title compound (15 mg, 55% yield at 0.02 mmol scale) as a white powder. ESI/MS m/z: 973 (M−H₂O+H).

### Preparation 58

### 2,2′-(4-(2-Amino-2-oxoethyl)-10-((4S,10S)-1-(4-(((R)-1-((2R,4S)-2-borono-4-hydroxypyrrolidin-1-yl)-1-oxopropan-2-yl)carbamoyl)phenyl)-4-(carboxymethyl)-10-(3-(3,3-dimethylguanidino)propyl)-3,6,9,12-tetraoxo-2,5,8,11-tetraazatridecan-13-yl)-1,4,7,10-tetraazacyclododecane-1,7-diyl)diacetic acid

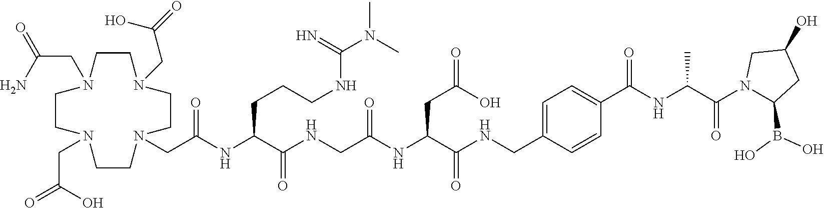 [SMILES: C[C@@H](NC(=O)c1ccc(CNC(=O)[C@H](CC(=O)O)NC(=O)CNC(=O)[C@H](CCCNC(=N)N(C)C)NC(=O)CN2CCN(CC(=O)O)CCN(CC(N)=O)CCN(CC(=O)O)CC2)cc1)C(=O)N1C[C@@H](O)C[C@H]1B(O)O]

4-((8S,14S,E)-8-(2-(7-(2-Amino-2-oxoethyl)-4,10-bis(2-(tert-butoxy)-2-oxoethyl)-1,4,7,10-tetraazacyclododecan-1-yl)acetamido)-14-(2-(tert-butoxy)-2-oxoethyl)-2-methyl-9,12,15-trioxo-3-((2,2,4,5,7-pentamethyl-2,3-dihydrobenzofuran)-6-sulfonamido)-2,4,10,13,16-pentaazaheptadec-3-en-17-yl)benzoic acid was coupled with ((2R,4S)-1-(D-alanyl)-4-hydroxypyrrolidin-2-yl)boronic acid HCl according to SP-5 and then all protecting groups removed (—Pbf, -tBu, and pinanediol) according to SP-6 to afford the title compound (3.5 mg, 25% yield at 0.01 mmol scale) as a white powder. ESI/MS m/z: 1060 (M−H₂O+H).

### Preparation 59

### 2,2′-(4-(2-Amino-2-oxoethyl)-10-(2-(((S)-17-((1R,4S)-4-(((R)-1-((2R,4S)-2-borono-4-hydroxypyrrolidin-1-yl)-1-oxopropan-2-yl)carbamoyl)cyclohexyl)-3-imino-2,10-dimethyl-9,15-dioxo-2,4,10,16-tetraazaheptadecan-8-yl)amino)-2-oxoethyl)-1,4,7,10-tetraazacyclododecane-1,7-diyl)diacetic acid

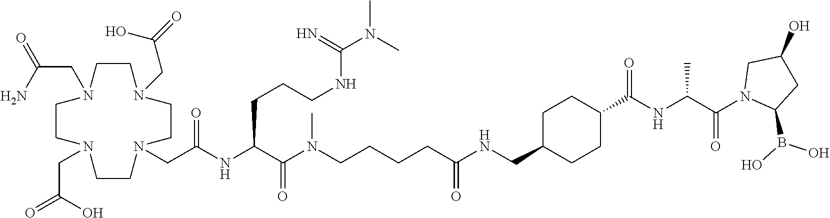 [SMILES: C[C@@H](NC(=O)[C@H]1CC[C@H](CNC(=O)CCCCN(C)C(=O)[C@H](CCCNC(=N)N(C)C)NC(=O)CN2CCN(CC(=O)O)CCN(CC(N)=O)CCN(CC(=O)O)CC2)CC1)C(=O)N1C[C@@H](O)C[C@H]1B(O)O]

(1SR,4SR)-4-((S,E)-8-(2-(7-(2-Amino-2-oxoethyl)-4,10-bis(2-(tert-butoxy)-2-oxoethyl)-1,4,7,10-tetraazacyclododecan-1-yl)acetamido)-2,10-dimethyl-9,15-dioxo-3-((2,2,4,6,7-pentamethyl-2,3-dihydrobenzofuran)-5-sulfonamido)-2,4,10,16-tetraazaheptadec-3-en-17-yl)cyclohexane-1-carboxylic acid was coupled with ((2R,4S)-1-(D-alanyl)-4-hydroxypyrrolidin-2-yl)boronic acid HCl according to SP-5 and then all protecting groups (—Pbf, -tBu, pinanediol) removed according to SP-6 to afford the title compound (12.8 mg, 46% yield at 0.02 mmol scale) as a white powder. ESI/MS m/z: 1007 (M−H₂O+H).

### Preparation 60

### 2,2′,2″-(10-(2-(((S)-1-((5-((4-(((R)-1-((2R,4S)-2-Borono-4-hydroxypyrrolidin-1-yl)-1-oxopropan-2-yl)carbamoyl)benzyl)(methyl)amino)-5-oxopentyl)amino)-5-guanidino-1-oxopentan-2-yl)amino)-2-oxoethyl)-1,4,7,10-tetraazacyclododecane-1,4,7-triyl)triacetic acid

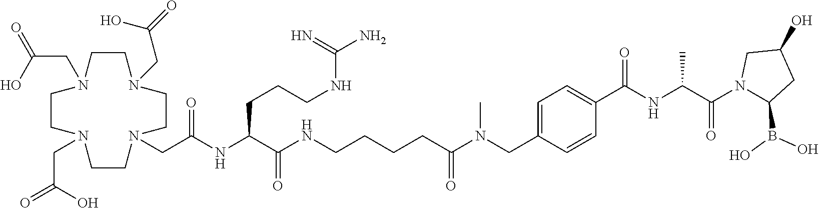 [SMILES: C[C@@H](NC(=O)c1ccc(CN(C)C(=O)CCCCNC(=O)[C@H](CCCNC(=N)N)NC(=O)CN2CCN(CC(=O)O)CCN(CC(=O)O)CCN(CC(=O)O)CC2)cc1)C(=O)N1C[C@@H](O)C[C@H]1B(O)O]

(S)-5-(5-(3-((2,2,4,5,7-Pentamethyl-2,3-dihydrobenzofuran-6-yl)sulfonyl)guanidino)-2-(2-(4,7,10-tris(2-(tert-butoxy)-2-oxoethyl)-1,4,7,10-tetraazacyclododecan-1-yl)acetamido)pentanamido)pentanoic acid was coupled with N-((2R)-1-((2R,4S)-4-hydroxy-2-((3aS,4S,6S)-3a,5,5-trimethylhexahydro-4,6-methanobenzo[d][1,3,2]dioxaborol-2-yl)pyrrolidin-1-yl)-1-oxopropan-2-yl)-4-((methylamino)methyl)benzamide HCl according to the SP-5 and then all the protecting groups (—Pbf, -tBu, and pinanediol) removed according to SP-6 to afford the title compound (20 mg, 15% yield at 0.1 mmol scale) as a white powder. ESI/MS m/z: 974 (MH-H₂O+H).

### Preparation 61

### 2,2′,2″-(10-(2-(((S)-1-((5-(4-(((R)-1-((R)-2-Boronopyrrolidin-1-yl)-1-oxopropan-2-yl)carbamoyl)-1-naphthamido)pentyl)amino)-5-guanidino-1-oxopentan-2-yl)amino)-2-oxoethyl)-1,4,7,10-tetraazacyclododecane-1,4,7-triyl)triacetic acid

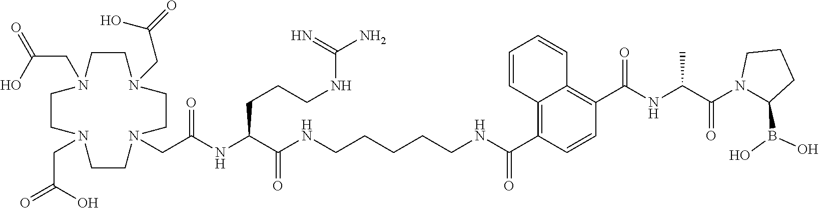 [SMILES: C[C@@H](NC(=O)c1ccc(C(=O)NCCCCCNC(=O)[C@H](CCCNC(=N)N)NC(=O)CN2CCN(CC(=O)O)CCN(CC(=O)O)CCN(CC(=O)O)CC2)c2ccccc12)C(=O)N1CCC[C@H]1B(O)O]

A solution of N¹-(5-((S)-2-amino-5-(3-((2,2,4,5,7-pentamethyl-2,3-dihydrobenzofuran-6-yl)sulfonyl)guanidino)pentanamido)pentyl)-N⁴-((2R)-1-oxo-1-((2R)-2-((3aS,4S,6S)-3a,5,5-trimethylhexahydro-4,6-methanobenzo[d][1,3,2]dioxaborol-2-yl)pyrrolidin-1-yl)propan-2-yl)naphthalene-1,4-dicarboxamide (56 mg, 0.05 mmol) in anhydrous DMF (1 mL) was treated with TEA (40 μL, 0.23 mmol) and 2,2′,2″-(10-(2-((2,5-dioxopyrrolidin-1-yl)oxy)-2-oxoethyl)-1,4,7,10-tetraazacyclododecane-1,4,7-triyl)triacetic acid (76 mg, 0.1 mmol) at 0° C. The mixture was stirred at rt overnight, concentrated under reduced pressure, and purified according to the General Procedure for Preparative HPLC purification (eluent gradient: 20% B for the first 3 min, then from 20% to 98% B over 12 min, which was maintained for the next 5 min). Both peaks of the desired compound and de-pinanediol compound were collected, concentrated under reduced pressure, and then the protecting groups (—Pbf and pinanediol) were removed according to SP-6 to afford the title compound (21.5 mg, 16% yield at 0.1 mmol scale) as a white powder. ESI/MS m/z: 994 (M−H₂O+H).

### Preparation 62

### 2,2′,2″-(10-((4S,10S)-1-(4-(((R)-1-((2R,4S)-2-Borono-4-hydroxypyrrolidin-1-yl)-1-oxopropan-2-yl)carbamoyl)phenyl)-4-(carboxymethyl)-10-(3-(3,3-dimethylguanidino)propyl)-3,6,9,12-tetraoxo-2,5,8,11-tetraazatridecan-13-yl)-1,4,7,10-tetraazacyclododecane-1,4,7-triyl)triacetic acid

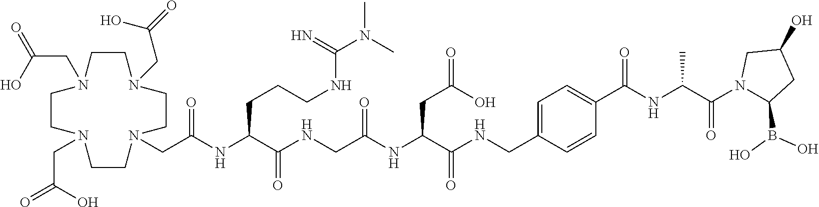 [SMILES: C[C@@H](NC(=O)c1ccc(CNC(=O)[C@H](CC(=O)O)NC(=O)CNC(=O)[C@H](CCCNC(=N)N(C)C)NC(=O)CN2CCN(CC(=O)O)CCN(CC(=O)O)CCN(CC(=O)O)CC2)cc1)C(=O)N1C[C@@H](O)C[C@H]1B(O)O]

4-((8S,14S,E)-14-(2-(tert-Butoxy)-2-oxoethyl)-2-methyl-9,12,15-trioxo-3-((2,2,4,5,7-pentamethyl-2,3-dihydrobenzofuran)-6-sulfonamido)-8-(2-(4,7,10-tris(2-(tert-butoxy)-2-oxoethyl)-1,4,7,10-tetraazacyclododecan-1-yl)acetamido)-2,4,10,13,16-pentaazaheptadec-3-en-17-yl)benzoic acid was coupled with ((2R,4S)-1-(D-alanyl)-4-hydroxypyrrolidin-2-yl)boronic acid HCl according to SP-5 and then the protecting groups (—Pbf, -tBu, and pinanediol) were removed according to SP-6 to afford the title compound (10.3 mg, 35% yield at 0.02 mmol scale) as a white powder. ESI/MS m/z: 1061 (M−H₂O+H).

### Preparation 63

### 2,2′,2″-(10-((10S,21S,24S,33R)-21-(4-(((S)-1-((5-((4-(((R)-1-((R)-2-Boronopyrrolidin-1-yl)-1-oxopropan-2-yl)carbamoyl)benzyl)amino)-5-oxopentyl)amino)-5-guanidino-1-oxopentan-2-yl)amino)-4-oxobutanamido)-1-(4-(((R)-1-((R)-2-boronopyrrolidin-1-yl)-1-oxopropan-2-yl)carbamoyl)phenyl)-24,33-dicarboxy-10-(3-guanidinopropyl)-3,9,12,15,22,30-hexaoxo-2,8,11,16,23,29-hexaazatritriacontan-33-yl)-1,4,7,10-tetraazacyclododecane-1,4,7-triyl)triacetic acid

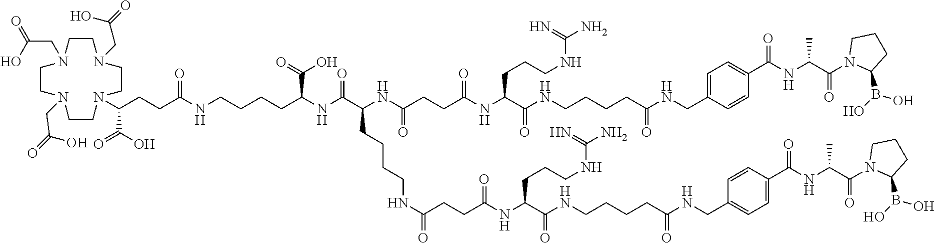 [SMILES: C[C@@H](NC(=O)c1ccc(CNC(=O)CCCCNC(=O)[C@H](CCCNC(=N)N)NC(=O)CCC(=O)NCCCC[C@H](NC(=O)CCC(=O)N[C@@H](CCCNC(=N)N)C(=O)NCCCCC(=O)NCc2ccc(C(=O)N[C@H](C)C(=O)N3CCC[C@H]3B(O)O)cc2)C(=O)N[C@@H](CCCCNC(=O)CC[C@H](C(=O)O)N2CCN(CC(=O)O)CCN(CC(=O)O)CCN(CC(=O)O)CC2)C(=O)O)cc1)C(=O)N1CCC[C@H]1B(O)O]

The initial resin loading with N²-(((9H-fluoren-9-yl)methoxy)carbonyl)-N⁶-(tert-butoxycarbonyl)-L-lysine was performed as described in SP-1. The Fmoc group was removed according to SP-3 and then coupled with N²,N⁶-bis(((9H-fluoren-9-yl)methoxy)carbonyl)-L-lysine according to SP-2. The Fmoc group was removed according to SP-3 and then coupled with 4-oxo-4-(((2S)-1-oxo-1-((5-oxo-5-((4-(((2R)-1-oxo-1-((2R)-2-((3aR,4S,6R)-3a,5,5-trimethyloctahydro-2H-4,6-methanobenzo[c]borol-2-yl)pyrrolidin-1-yl)propan-2-yl)carbamoyl)benzyl)amino)pentyl)amino)-5-(3-((2,2,4,6,7-pentamethyl-2,3-dihydrobenzofuran-5-yl)sulfonyl)guanidino)pentan-2-yl)amino)butanoic acid according to SP-2 using PyBroP as the coupling reagent. The compound was cleaved from the resin according to SP-4 (4N HCl in 1,4-dioxane was added here to ensure the Boc protection at Lys was completely removed) and was then coupled with (R)-2,2′,2″-(10-(1-carboxy-4-(4-nitrophenoxy)-4-oxobutyl)-1,4,7,10-tetraazacyclododecane-1,4,7-triyl)triacetic acid (2.0 eq.) in anhydrous DMF with 10 eq. of DIPEA. The remaining protecting groups (—Pbf and pinanediol) were removed according to SP-6 (in this case, the reaction of the pinanediol removal needed to be further treated with PhB(OH)₂ to be removed completely) to afford the title compound (3.5 mg, 1.4%) as a white powder. ESI/MS m/z: 1992 (M-3H₂O+H).

### Preparation 64

### 2,2′,2″-(10-((10S,20S,29R)-1-(4-(((R)-1-((R)-2-Boronopyrrolidin-1-yl)-1-oxopropan-2-yl)carbamoyl)phenyl)-20,29-dicarboxy-10-(3-guanidinopropyl)-3,9,12,15,18,26-hexaoxo-2,8,11,16,19,25-hexaazanonacosan-29-yl)-1,4,7,10-tetraazacyclododecane-1,4,7-triyl)triacetic acid

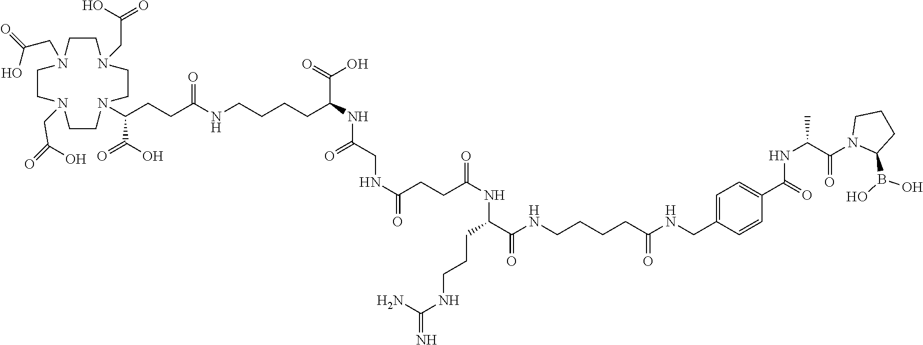 [SMILES: C[C@@H](NC(=O)c1ccc(CNC(=O)CCCCNC(=O)[C@H](CCCNC(=N)N)NC(=O)CCC(=O)NCC(=O)N[C@@H](CCCCNC(=O)CC[C@H](C(=O)O)N2CCN(CC(=O)O)CCN(CC(=O)O)CCN(CC(=O)O)CC2)C(=O)O)cc1)C(=O)N1CCC[C@H]1B(O)O]

The initial resin loading with N²-(((9H-fluoren-9-yl)methoxy)carbonyl)-N⁶-(tert-butoxycarbonyl)-L-lysine was performed as described in SP-1. The Fmoc group was removed according to SP-3 and then coupled with (((9H-fluoren-9-yl)methoxy)carbonyl)glycine according to SP-2. The Fmoc group was removed according to SP-3 and then coupled with 4-oxo-4-(((2S)-1-oxo-1-((5-oxo-5-((4-(((2R)-1-oxo-1-((2R)-2-((3aR,4S,6R)-3a,5,5-trimethyloctahydro-2H-4,6-methanobenzo[c]borol-2-yl)pyrrolidin-1-yl)propan-2-yl)carbamoyl)benzyl)amino)pentyl)amino)-5-(3-((2,2,4,6,7-pentamethyl-2,3-dihydrobenzofuran-5-yl)sulfonyl)guanidino)pentan-2-yl)amino)butanoic acid according to SP-2 but using PyBroP as the coupling reagent. The peptide was cleaved from the resin according to SP-4 (4N HCl in 1,4-dioxane was added here to ensure the Boc protection at Lys was completely removed) and then coupled with (R)-2,2′,2″-(10-(1-carboxy-4-(4-nitrophenoxy)-4-oxobutyl)-1,4,7,10-tetraazacyclododecane-1,4,7-triyl)triacetic acid (2.0 eq.) in anhydrous DMF with 10 eq. of DIPEA. The remaining protecting groups (—Pbf and pinanediol) were removed according to SP-6 (in this case, the reaction of the pinanediol removal needed to be further treated with PhB(OH)₂ to be removed completely) to afford the title compound (27.4 mg, 16%) as a white powder. ESI/MS m/z: 1301 (M−H₂O+H).

### Preparation 65

### 2,2′,2″-(10-((10S,20S,29R)-1-(4-(((R)-1-((R)-2-Boronopyrrolidin-1-yl)-1-oxopropan-2-yl)carbamoyl)phenyl)-20,29-dicarboxy-10-(3-guanidinopropyl)-8-methyl-3,9,12,15,18,26-hexaoxo-2,8,11,16,19,25-hexaazanonacosan-29-yl)-1,4,7,10-tetraazacyclododecane-1,4,7-triyl)triacetic acid

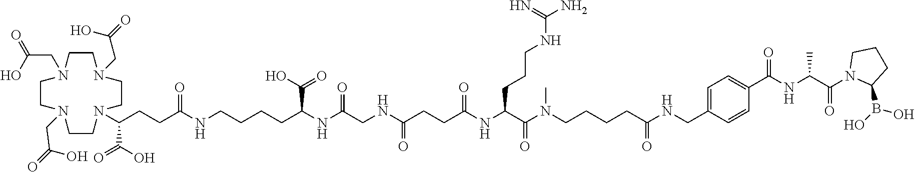 [SMILES: C[C@@H](NC(=O)c1ccc(CNC(=O)CCCCN(C)C(=O)[C@H](CCCNC(=N)N)NC(=O)CCC(=O)NCC(=O)N[C@@H](CCCCNC(=O)CC[C@H](C(=O)O)N2CCN(CC(=O)O)CCN(CC(=O)O)CCN(CC(=O)O)CC2)C(=O)O)cc1)C(=O)N1CCC[C@H]1B(O)O]

(4-(((2S)-1-(Methyl(5-oxo-5-((4-(((2R)-1-oxo-1-((2R)-2-((3aS,4S,6S)-3a,5,5-trimethylhexahydro-4,6-methanobenzo[d][1,3,2]dioxaborol-2-yl)pyrrolidin-1-yl)propan-2-yl)carbamoyl)benzyl)amino)pentyl)amino)-1-oxo-5-(3-((2,2,4,6,7-pentamethyl-2,3-dihydrobenzofuran-5-yl)sulfonyl)guanidino)pentan-2-yl)amino)-4-oxobutanoyl)glycine was coupled with tert-butyl N²,N⁶-bis(tert-butoxycarbonyl)-L-lysinate with HATU according to SP-5, then was de-protected with 4N HCl in 1,4-dioxane and then conjugated with (R)-2,2′,2″-(10-(1-carboxy-4-(4-nitrophenoxy)-4-oxobutyl)-1,4,7,10-tetraazacyclododecane-1,4,7-triyl)triacetic acid (2.0 eq.) in anhydrous DMF with 10 eq. of DIPEA. The —Pbf and pinanediol protective groups were removed according to SP-6 to afford the title compound (12.7 mg, 16% yield at 0.05 mmol scale) as a white powder. ESI/MS m/z: 1315 (M−H₂O+H).

### Preparation 66

### 2,2′,2″-(10-((10R,20S,29R)-1-(4-(((R)-1-((R)-2-Boronopyrrolidin-1-yl)-1-oxopropan-2-yl)carbamoyl)phenyl)-20,29-dicarboxy-10-(3-guanidinopropyl)-3,8,11,15,18,26-hexaoxo-2,9,12,16,19,25-hexaazanonacosan-29-yl)-1,4,7,10-tetraazacyclododecane-1,4,7-triyl)triacetic acid

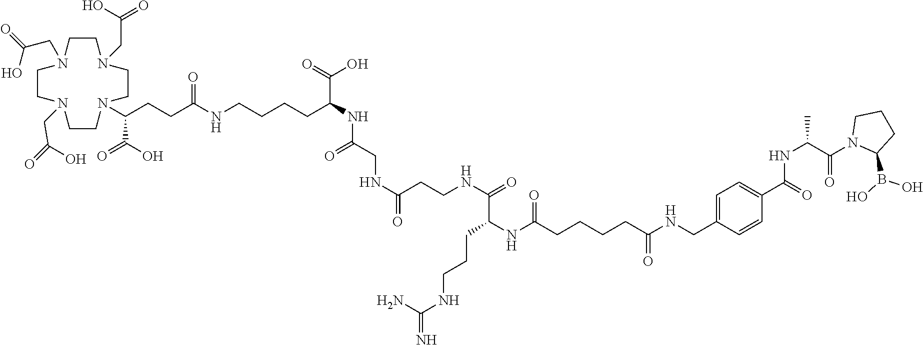 [SMILES: C[C@@H](NC(=O)c1ccc(CNC(=O)CCCCC(=O)N[C@H](CCCNC(=N)N)C(=O)NCCC(=O)NCC(=O)N[C@@H](CCCCNC(=O)CC[C@H](C(=O)O)N2CCN(CC(=O)O)CCN(CC(=O)O)CCN(CC(=O)O)CC2)C(=O)O)cc1)C(=O)N1CCC[C@H]1B(O)O]

The initial resin loading with N²-(((9H-fluoren-9-yl)methoxy)carbonyl)-N⁶-(tert-butoxycarbonyl)-L-lysine was performed as described in SP-1. The Fmoc group was removed according to SP-3 and then coupled with (((9H-fluoren-9-yl)methoxy)carbonyl)glycine according to SP-2. The Fmoc group was removed according to SP-3 and then coupled with 3-((((9H-fluoren-9-yl)methoxy)carbonyl)amino)propanoic acid according to SP-2. The Fmoc group was removed according to SP-3 and then coupled with (R)-2-(l²-azaneyl)-5-(3-((2,2,4,6,7-pentamethyl-2,3-dihydrobenzofuran-5-yl)sulfonyl)guanidino)pentanoic acid according to SP-2. The Fmoc group was removed according to SP-3 and then coupled with 6-oxo-6-((4-(((2R)-1-oxo-1-((2R)-2-((3aS,4S,6S)-3a,5,5-trimethylhexahydro-4,6-methanobenzo[d][1,3,2]dioxaborol-2-yl)pyrrolidin-1-yl)propan-2-yl)carbamoyl)benzyl)amino)hexanoic acid (which was synthesized by coupling of 6-(tert-butoxy)-6-oxohexanoic acid to 4-(aminomethyl)-N-((2R)-1-oxo-1-((2R)-2-((3aS,4S,6S)-3a,5,5-trimethylhexahydro-4,6-methanobenzo[d][1,3,2]dioxaborol-2-yl)pyrrolidin-1-yl)propan-2-yl)benzamide HCl with HA TU, DIPEA in DMF; and then the tert-butyl ester was removed with 4N HCl in 1,4-dioxane) according to SP-2. The compound was cleaved from the resin according to SP-4 (4N HCl in 1,4-dioxane was added to ensure the Boc protection at Lys was completely removed) and then coupled with (R)-DOTAGA-PNP (2.0 eq.) in anhydrous DMF with 10 eq. of DIPEA. The —Pbf and pinanediol protective groups were removed according to SP-6 to afford the title compound (7.4 mg, 5%) as a white powder. ESI/MS m/z: 1301 (M−H₂O+H).

### Preparation 67

### 2,2′,2″-(10-((10S,20S,29R)-1-(4-(((R)-1-((R)-2-Boronopyrrolidin-1-yl)-1-oxopropan-2-yl)carbamoyl)phenyl)-20,29-dicarboxy-10-(3-guanidinopropyl)-11-methyl-3,9,12,15,18,26-hexaoxo-2,8,11,16,19,25-hexaazanonacosan-29-yl)-1,4,7,10-tetraazacyclododecane-1,4,7-triyl)triacetic acid

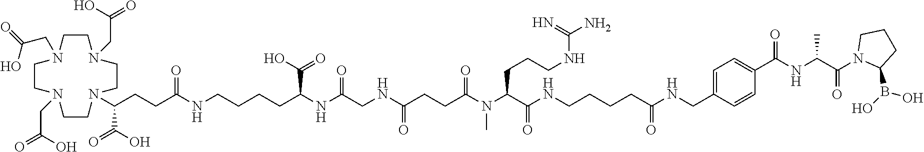 [SMILES: C[C@@H](NC(=O)c1ccc(CNC(=O)CCCCNC(=O)[C@H](CCCNC(=N)N)N(C)C(=O)CCC(=O)NCC(=O)N[C@@H](CCCCNC(=O)CC[C@H](C(=O)O)N2CCN(CC(=O)O)CCN(CC(=O)O)CCN(CC(=O)O)CC2)C(=O)O)cc1)C(=O)N1CCC[C@H]1B(O)O]

(S)-2,2,11-Trimethyl-4,7,10,13-tetraoxo-12-(3-(3-((2,2,4,6,7-pentamethyl-2,3-dihydrobenzofuran-5-yl)sulfonyl)guanidino)propyl)-3-oxa-6,11,14-triazanonadecan-19-oic acid was coupled to 4-((l²-azaneyl)methyl)-N-((2R)-1-oxo-1-((2R)-2-((3aS,4S,6S)-3a,5,5-trimethylhexahydro-4,6-methanobenzo[d][1,3,2]dioxaborol-2-yl)pyrrolidin-1-yl)propan-2-yl)benzamide according to SP-5. The t-butyl ester protecting group was removed with 4N HCl in 1,4-dioxane and then coupled to preloaded H-Lys(Boc)-ClTrt-resin according to SP-2. The compound was cleaved from the resin according to SP-4 (4N HCl in 1,4-dioxane was added to ensure the Boc protection at Lys was completely removed) and then coupled with (R)-2,2′,2″-(10-(1-carboxy-4-(4-nitrophenoxy)-4-oxobutyl)-1,4,7,10-tetraazacyclododecane-1,4,7-triyl)triacetic acid (2.0 eq.) in anhydrous DMF with 10 eq. of DIPEA. The —Pbf and pinanediol protecting groups were removed according to SP-6 to afford the title compound (19.4 mg, 11%) as a white powder. ESI/MS m/z: 1315 (M−H₂O+H).

### Preparation 68

### 2,2′,2″-(10-((10S,20S,29R)-1-(4-(((R)-1-((R)-2-Boronopyrrolidin-1-yl)-1-oxopropan-2-yl)carbamoyl)phenyl)-20,29-dicarboxy-10-(3-guanidinopropyl)-3,9,12,18,26-pentaoxo-2,8,11,19,25-pentaazanonacosan-29-yl)-1,4,7,10-tetraazacyclododecane-1,4,7-triyl)triacetic acid

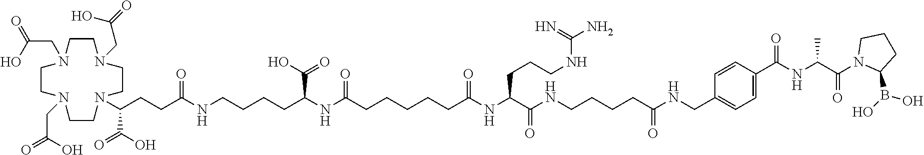 [SMILES: C[C@@H](NC(=O)c1ccc(CNC(=O)CCCCNC(=O)[C@H](CCCNC(=N)N)NC(=O)CCCCCC(=O)N[C@@H](CCCCNC(=O)CC[C@H](C(=O)O)N2CCN(CC(=O)O)CCN(CC(=O)O)CCN(CC(=O)O)CC2)C(=O)O)cc1)C(=O)N1CCC[C@H]1B(O)O]

(S)-5-(2-(7-(tert-Butoxy)-7-oxoheptanamido)-5-(3-((2,2,4,6,7-pentamethyl-2,3-dihydrobenzofuran-5-yl)sulfonyl)guanidino)pentanamido)pentanoic acid was coupled to 4-(aminomethyl)-N-((2R)-1-oxo-1-((2R)-2-((3aS,4S,6S)-3a,5,5-trimethylhexahydro-4,6-methanobenzo[d][1,3,2]dioxaborol-2-yl)pyrrolidin-1-yl)propan-2-yl)benzamide according to the SP-5. The t-butyl ester protecting group was removed with 4N HCl in 1,4-dioxane to give 7-oxo-7-(((2S)-1-oxo-1-((5-oxo-5-((4-(((2R)-1-oxo-1-((2R)-2-((3aS,4S,6S)-3a,5,5-trimethylhexahydro-4,6-methanobenzo[d][1,3,2]dioxaborol-2-yl)pyrrolidin-1-yl)propan-2-yl)carbamoyl)benzyl)amino)pentyl)amino)-5-(3-((2,2,4,6,7-pentamethyl-2,3-dihydrobenzofuran-5-yl)sulfonyl)guanidino)pentan-2-yl)amino)heptanoic acid which was coupled to preloaded H-Lys(Boc)-ClTrt-resin according to SP-2 except using PyBroP instead of HATU as the coupling reagent. The compound was cleaved from the resin according to SP-4 (4N HCl in 1,4-dioxane was added here to ensure the Boc protection at Lys was completely removed) and then conjugated with (R)-2,2′,2″-(10-(1-carboxy-4-(4-nitrophenoxy)-4-oxobutyl)-1,4,7,10-tetraazacyclododecane-1,4,7-triyl)triacetic acid (2.0 eq.) in anhydrous DMF with 10 eq. of DIPEA. The —Pbf and pinanediol protecting groups were removed according to SP-6 to afford the title compound (10 mg, 6%) as a white powder. ESI/MS m/z: 1286 (M−H₂O+H).

### Preparation 69

### 2,2′,2″-(10-((10S,20S,29R)-1-(4-(((R)-1-((R)-2-Boronopyrrolidin-1-yl)-1-oxopropan-2-yl)carbamoyl)phenyl)-20,29-dicarboxy-10-(3-(3,3-dimethylguanidino)propyl)-3,9,12,18,26-pentaoxo-2,8,11,19,25-pentaazanonacosan-29-yl)-1,4,7,10-tetraazacyclododecane-1,4,7-triyl)triacetic acid

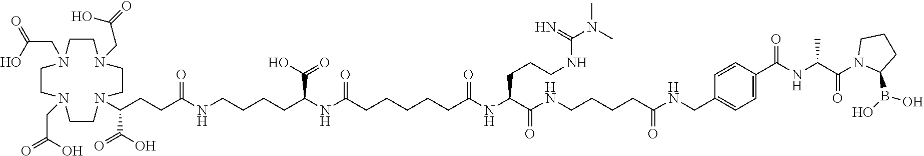 [SMILES: C[C@@H](NC(=O)c1ccc(CNC(=O)CCCCNC(=O)[C@H](CCCNC(=N)N(C)C)NC(=O)CCCCCC(=O)N[C@@H](CCCCNC(=O)CC[C@H](C(=O)O)N2CCN(CC(=O)O)CCN(CC(=O)O)CCN(CC(=O)O)CC2)C(=O)O)cc1)C(=O)N1CCC[C@H]1B(O)O]

The initial resin loading with 4-(((((9H-fluoren-9-yl)methoxy)carbonyl)amino)methyl)benzoic acid was performed as described in SP-1. The Fmoc group was removed according to SP-3 and then coupled with 5-((((9H-fluoren-9-yl)methoxy)carbonyl)amino)pentanoic acid according to SP-2 except using PyBroP instead of HATU as the coupling reagent. The Fmoc group was removed according to SP-3 and then coupled with (Z)-N²-(((9H-fluoren-9-yl)methoxy)carbonyl)-N^{w},N-dimethyl-N^{w′}-((2,2,4,6,7-pentamethyl-2,3-dihydrobenzofuran-5-yl)sulfonyl)-L-arginine according to SP-2. The Fmoc group was removed according to SP-3 and then reacted with 7-(4-nitrophenoxy)-7-oxoheptanoic acid in DMF and DIPEA. The remaining carboxylic acid was coupled with tert-butyl N⁶-(((9H-fluoren-9-yl)methoxy)carbonyl)-L-lysinate according to SP-2 except using PyBroP instead of HATU as the coupling reagent. The Fmoc group was removed according to SP-3 and then coupled with (R)-5-(tert-butoxy)-5-oxo-4-(4,7,10-tris(2-(tert-butoxy)-2-oxoethyl)-1,4,7,10-tetraazacyclododecan-1-yl)pentanoic acid according to SP-2 except using PyBroP instead of HATU as the coupling reagent. The compound was cleaved from the resin according to SP-4 and then coupled to (2R)-2-(l²-azaneyl)-1-((2R)-2-((3aS,4S,6S)-3a,5,5-trimethylhexahydro-4,6-methanobenzo[d][1,3,2]dioxaborol-2-yl)pyrrolidin-1-yl)propan-1-one according to the SP-5. The —Pbf, -tBu, and pinanediol protecting groups were removed according to SP-6 to afford the title compound (12.8 mg, 8%) as a white powder. ESI/MS m/z: 1315 (M−H₂O+H).

### Preparation 70

### 2,2′,2″-(10-((10S,20S,29R)-1-(4-(((R)-1-((2R,4S)-2-Borono-4-hydroxypyrrolidin-1-yl)-1-oxopropan-2-yl)carbamoyl)phenyl)-20,29-dicarboxy-10-(3-(3,3-dimethylguanidino)propyl)-3,9,12,18,26-pentaoxo-2,8,11,19,25-pentaazanonacosan-29-yl)-1,4,7,10-tetraazacyclododecane-1,4,7-triyl)triacetic acid

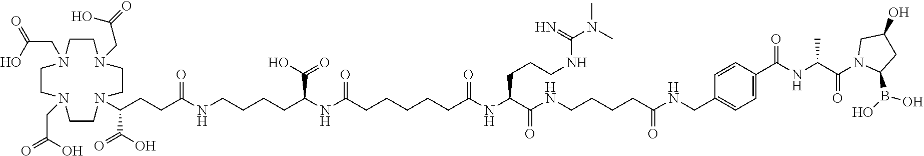 [SMILES: C[C@@H](NC(=O)c1ccc(CNC(=O)CCCCNC(=O)[C@H](CCCNC(=N)N(C)C)NC(=O)CCCCCC(=O)N[C@@H](CCCCNC(=O)CC[C@H](C(=O)O)N2CCN(CC(=O)O)CCN(CC(=O)O)CCN(CC(=O)O)CC2)C(=O)O)cc1)C(=O)N1C[C@@H](O)C[C@H]1B(O)O]

The initial resin loading with 4-(((((9H-fluoren-9-yl)methoxy)carbonyl)amino)methyl)benzoic acid was performed as described in SP-1. The Fmoc group was removed according to SP-3 and then coupled with 5-((((9H-fluoren-9-yl)methoxy)carbonyl)amino)pentanoic acid according to SP-2 except using PyBroP instead of HATU as the coupling reagent. The Fmoc group was removed according to SP-3 and then coupled with (Z)-N²-(((9H-fluoren-9-yl)methoxy)carbonyl)-N^{w},N^{w}-dimethyl-N^{w′}-((2,2,4,6,7-pentamethyl-2,3-dihydrobenzofuran-5-yl)sulfonyl)-L-arginine according to SP-2. The Fmoc group was removed according to SP-3 and then reacted with 7-(4-nitrophenoxy)-7-oxoheptanoic acid in DMF and DIPEA. The free carboxylic acid was coupled with tert-butyl N⁶-(((9H-fluoren-9-yl)methoxy)carbonyl)-L-lysinate according to SP-2 except using PyBroP instead of HATU as the coupling reagent. The Fmoc group was removed according to SP-3 and then coupled with (R)-5-(tert-Butoxy)-5-oxo-4-(4,7,10-tris(2-(tert-butoxy)-2-oxoethyl)-1,4,7,10-tetraazacyclododecan-1-yl)pentanoic acid according to SP-2 except using PyBroP instead of HATU as the coupling reagent. The compound was cleaved from the resin according to SP-4 and then coupled to (2R)-2-(l²-azaneyl)-1-((2R,4S)-4-hydroxy-2-((3aS,4S,6S)-3a,5,5-trimethylhexahydro-4,6-methanobenzo[d][1,3,2]dioxaborol-2-yl)pyrrolidin-1-yl)propan-1-one HCl according to SP-5. The —Pbf, -tBu, and pinanediol protecting groups were removed according to SP-6 to afford the title compound (4.9 mg, 3%) as a white powder. ESI/MS m/z: 1330 (M−H₂O+H).

### Preparation 71

### 2,2′,2″-(10-((10S,29R)-1-(4-(((R)-1-((R)-2-Boronopyrrolidin-1-yl)-1-oxopropan-2-yl)carbamoyl)phenyl)-29-carboxy-10-(3-(3,3-dimethylguanidino)propyl)-3,9,12,18,26-pentaoxo-2,8,11,19,25-pentaazanonacosan-29-yl)-1,4,7,10-tetraazacyclododecane-1,4,7-triyl)triacetic acid

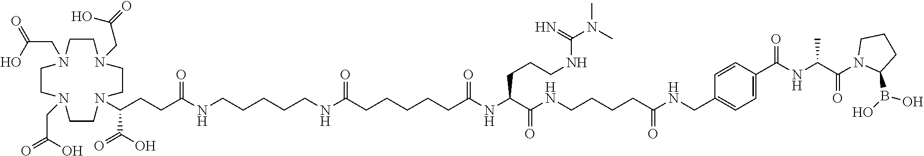 [SMILES: C[C@@H](NC(=O)c1ccc(CNC(=O)CCCCNC(=O)[C@H](CCCNC(=N)N(C)C)NC(=O)CCCCCC(=O)NCCCCCNC(=O)CC[C@H](C(=O)O)N2CCN(CC(=O)O)CCN(CC(=O)O)CCN(CC(=O)O)CC2)cc1)C(=O)N1CCC[C@H]1B(O)O]

The tert-butyl group on tert-butyl 7-(((8S,Z)-2-methyl-9,15-dioxo-17-(4-(((2R)-1-oxo-1-((2R)-2-((3aS,4S,6S)-3a,5,5-trimethylhexahydro-4,6-methanobenzo[d][1,3,2]dioxaborol-2-yl)pyrrolidin-1-yl)propan-2-yl)carbamoyl)phenyl)-3-(((2,2,4,6,7-pentamethyl-2,3-dihydrobenzofuran-5-yl)sulfonyl)imino)-2,4,10,16-tetraazaheptadecan-8-yl)amino)-7-oxoheptanoate was removed with 4N HCl in 1,4-dioxane and then coupled with tert-butyl (5-aminopentyl)carbamate with HATU and DIPEA in DMF. The Boc group was removed using TFA in DCM and then coupled with (R)-2,2′,2″-(10-(1-carboxy-4-(4-nitrophenoxy)-4-oxobutyl)-1,4,7,10-tetraazacyclododecane-1,4,7-triyl)triacetic acid (2.0 eq.) in anhydrous DMF with 10 eq. of DIPEA. The pinanediol protecting group was removed with PhB(OH)₂ in MTBE-ACN-0.01N HCl to afford the title compound (10.0 mg, 30% yield at 0.02 mmol scale) as a white powder. ESI/MS m/z: 1270 (M−H₂O+H).

### Preparation 72

### 2,2′,2″-(10-((10S,21R)-1-(4-(((R)-1-((R)-2-Boronopyrrolidin-1-yl)-1-oxopropan-2-yl)carbamoyl)phenyl)-21-carboxy-10-(3-(3,3-dimethylguanidino)propyl)-3,9,12,18-tetraoxo-2,8,11,17-tetraazahenicosan-21-yl)-1,4,7,10-tetraazacyclododecane-1,4,7-triyl)triacetic acid

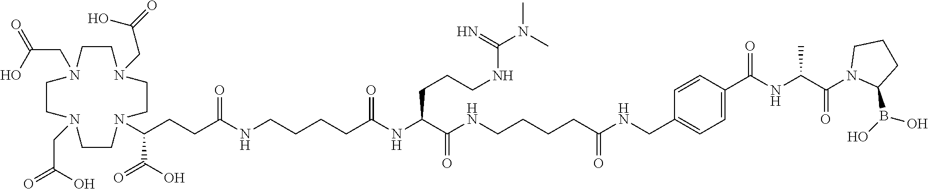 [SMILES: C[C@@H](NC(=O)c1ccc(CNC(=O)CCCCNC(=O)[C@H](CCCNC(=N)N(C)C)NC(=O)CCCCNC(=O)CC[C@H](C(=O)O)N2CCN(CC(=O)O)CCN(CC(=O)O)CCN(CC(=O)O)CC2)cc1)C(=O)N1CCC[C@H]1B(O)O]

((R)-1-((4-((S)-8-(5-Aminopentanamido)-3-imino-2-methyl-9,15-dioxo-2,4,10,16-tetraazaheptadecan-17-yl)benzoyl)-D-alanyl)pyrrolidin-2-yl)boronic acid was coupled with (R)-2,2′,2″-(10-(1-carboxy-4-(4-nitrophenoxy)-4-oxobutyl)-1,4,7,10-tetraazacyclododecane-1,4,7-triyl)triacetic acid (2.0 eq.) in anhydrous DMF with 10 eq. of DIPEA to afford the title compound (4.5 mg, 30% yield at 0.01 mmol scale) as a white powder. ESI/MS m/z: 1143 (M−H₂O+H).

### Preparation 73

### 2,2′,2″-(10-((10S,20S,29R)-1-(4-(((R)-1-((R)-2-Boronopyrrolidin-1-yl)-1-oxopropan-2-yl)carbamoyl)naphthalen-1-yl)-20,29-dicarboxy-10-(3-guanidinopropyl)-1,9,12,18,26-pentaoxo-2,8,11,19,25-pentaazanonacosan-29-yl)-1,4,7,10-tetraazacyclododecane-1,4,7-triyl)triacetic acid

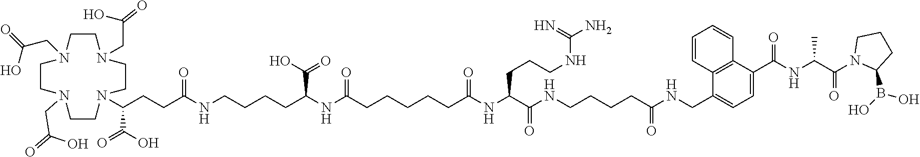 [SMILES: C[C@@H](NC(=O)c1ccc(CNC(=O)CCCCNC(=O)[C@H](CCCNC(=N)N)NC(=O)CCCCCC(=O)N[C@@H](CCCCNC(=O)CC[C@H](C(=O)O)N2CCN(CC(=O)O)CCN(CC(=O)O)CCN(CC(=O)O)CC2)C(=O)O)c2ccccc12)C(=O)N1CCC[C@H]1B(O)O]

(S)-4-((5-(2-(7-(tert-Butoxy)-7-oxoheptanamido)-5-(3-((2,2,4,6,7-pentamethyl-2,3-dihydrobenzofuran-5-yl)sulfonyl)guanidino)pentanamido)pentyl)carbamoyl)-1-naphthoic acid was coupled to (2R)-2-(l²-azaneyl)-1-((2R)-2-((3aS,4S,6S)-3a,5,5-trimethylhexahydro-4,6-methanobenzo[d][1,3,2]dioxaborol-2-yl)pyrrolidin-1-yl)propan-1-one according to SP-5. After removal of the tert-butyl ester protecting group, the free carboxylic acid was coupled to tert-butyl N⁶-(tert-butoxycarbonyl)-L-lysinate with HATU and DIPEA in DMF. After removal of the Boc protecting group with 4N HCl in 1,4-dioxane, the free amine was then coupled with (R)-2,2′,2″-(10-(1-carboxy-4-(4-nitrophenoxy)-4-oxobutyl)-1,4,7,10-tetraazacyclododecane-1,4,7-triyl)triacetic acid (2.0 eq.) in anhydrous DMF with 10 eq. of DIPEA. The remaining protecting groups were removed according to SP-6 to give the title compound (7.0 mg, 20% yield at 0.02 mmol scale) as a white powder. ESI/MS m/z: 1336 (M−H₂O+H).

### Preparation 74

### 2,2′,2″-(10-((10S,20S)-1-(4-(((R)-1-((R)-2-Boronopyrrolidin-1-yl)-1-oxopropan-2-yl)carbamoyl)naphthalen-1-yl)-20-carboxy-10-(3-guanidinopropyl)-1,9,12,18,26-pentaoxo-2,8,11,19,25-pentaazaheptacosan-27-yl)-1,4,7,10-tetraazacyclododecane-1,4,7-triyl)triacetic acid

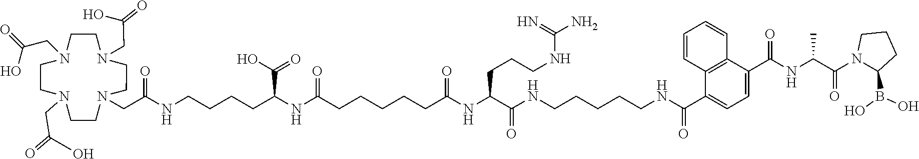 [SMILES: C[C@@H](NC(=O)c1ccc(C(=O)NCCCCCNC(=O)[C@H](CCCNC(=N)N)NC(=O)CCCCCC(=O)N[C@@H](CCCCNC(=O)CN2CCN(CC(=O)O)CCN(CC(=O)O)CCN(CC(=O)O)CC2)C(=O)O)c2ccccc12)C(=O)N1CCC[C@H]1B(O)O]

(S)-4-((5-(2-(7-(tert-Butoxy)-7-oxoheptanamido)-5-(3-((2,2,4,6,7-pentamethyl-2,3-dihydrobenzofuran-5-yl)sulfonyl)guanidino)pentanamido)pentyl)carbamoyl)-1-naphthoic acid was coupled to (2R)-2-(l²-azaneyl)-1-((2R)-2-((3aS,4S,6S)-3a,5,5-trimethylhexahydro-4,6-methanobenzo[d][1,3,2]dioxaborol-2-yl)pyrrolidin-1-yl)propan-1-one according to SP-5. After removal of the tert-butyl ester protecting group, the free carboxylic acid was coupled to tert-butyl N⁶-(tert-butoxycarbonyl)-L-lysinate with HATU and DIPEA in DMF. After removal of the Boc protecting group with 4N HCl in 1,4-dioxane, the free amine was then conjugated with 2,2′,2″-(10-(2-((2,5-dioxopyrrolidin-1-yl)oxy)-2-oxoethyl)-1,4,7,10-tetraazacyclododecane-1,4,7-triyl)triacetic acid (2.0 eq.) in anhydrous DMF with 10 eq. of DIPEA. The remaining protecting groups (—Pbf, -tBu, and pinanediol) were removed according to SP-6 to afford the title compound (7.1 mg, 20% yield at 0.02 mmol scale) as a white powder. ESI/MS m/z: 1264 (M−H₂O+H).

### Preparation 75

### 2,2′-(7-(2-(l¹-Oxidaneyl)-2-(l³-oxidaneylidene)ethyl)-10-(2-(((S)-1-((5-((4-(((R)-1-((R)-2-boronopyrrolidin-1-yl)-1-oxopropan-2-yl)carbamoyl)benzyl)amino)-5-oxopentyl)amino)-5-guanidino-1-oxopentan-2-yl)amino)-2-oxoethyl)-1,4,7,10-tetraazacyclododecane-1,4-diyl)diacetic acid

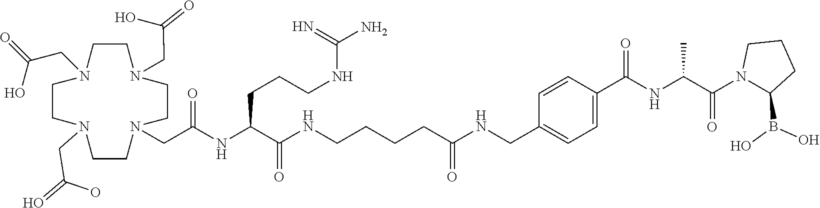 [SMILES: C[C@@H](NC(=O)c1ccc(CNC(=O)CCCCNC(=O)[C@H](CCCNC(=N)N)NC(=O)CN2CCN(CC(=O)O)CCN(CC(=O)O)CCN(CC(=O)O)CC2)cc1)C(=O)N1CCC[C@H]1B(O)O]

4-((5-((S)-2-(l²-Azaneyl)-5-(3-((2,2,4,6,7-pentamethyl-2,3-dihydrobenzofuran-5-yl)sulfonyl)guanidino)pentanamido)pentanamido)methyl)-N-((2R)-1-oxo-1-((2R)-2-((3aS,4S,6S)-3a,5,5-trimethylhexahydro-4,6-methanobenzo[d][1,3,2]dioxaborol-2-yl)pyrrolidin-1-yl)propan-2-yl)benzamide was coupled with 2,2′,2″-(10-(2-((2,5-dioxopyrrolidin-1-yl)oxy)-2-oxoethyl)-1,4,7,10-tetraazacyclododecane-1,4,7-triyl)triacetic acid (2.0 eq.) in anhydrous DMF with 10 eq. of DIPEA. The remaining —Pbf and pinanediol protecting groups were removed according to SP-6 to afford the title compound (16.0 mg, 24% yield at 0.05 mmol scale) as a white powder. ESI/MS m/z: 944 (M−H₂O+H).

### Preparation 76

### 2,2′,2″-(10-(2-(((S)-1-((5-((4-(((R)-1-((2R,4S)-2-Borono-4-hydroxypyrrolidin-1-yl)-1-oxopropan-2-yl)carbamoyl)benzyl)amino)-5-oxopentyl)amino)-5-guanidino-1-oxopentan-2-yl)amino)-2-oxoethyl)-1,4,7,10-tetraazacyclododecane-1,4,7-triyl)triacetic acid

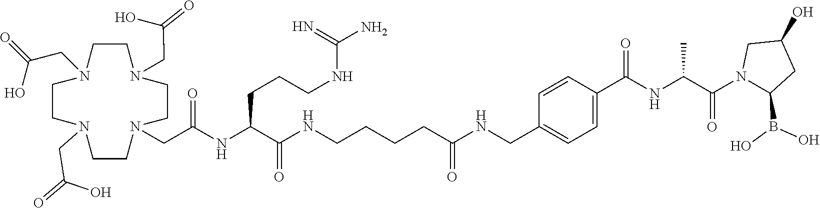 [SMILES: C[C@@H](NC(=O)c1ccc(CNC(=O)CCCCNC(=O)[C@H](CCCNC(=N)N)NC(=O)CN2CCN(CC(=O)O)CCN(CC(=O)O)CCN(CC(=O)O)CC2)cc1)C(=O)N1C[C@@H](O)C[C@H]1B(O)O]

4-((5-((S)-2-Amino-5-(3-((2,2,4,6,7-pentamethyl-2,3-dihydrobenzofuran-5-yl)sulfonyl)guanidino)pentanamido)pentanamido)methyl)-N-((2R)-1-((2R,4S)-4-hydroxy-2-((3aS,4S,6S)-3a,5,5-trimethylhexahydro-4,6-methanobenzo[d][1,3,2]dioxaborol-2-yl)pyrrolidin-1-yl)-1-oxopropan-2-yl)benzamide was coupled to 2,2′,2″-(10-(2-((2,5-dioxopyrrolidin-1-yl)oxy)-2-oxoethyl)-1,4,7,10-tetraazacyclododecane-1,4,7-triyl)triacetic acid (2.0 eq.) in anhydrous DMF with 10 eq. of DIPEA. The —Pbf and pinanediol protecting groups were removed according to SP-6 to afford the title compound (9.0 mg, 35% yield at 0.02 mmol scale) as a white powder. ESI/MS m/z: 960 (M−H₂O+H).

### Preparation 77

### 2,2′,2″-(10-(2-(((S)-17-(4-(((R)-1-((2R,4S)-2-Borono-4-hydroxypyrrolidin-1-yl)-1-oxopropan-2-yl)carbamoyl)phenyl)-3-imino-2-methyl-9,15-dioxo-2,4,10,16-tetraazaheptadecan-8-yl)amino)-2-oxoethyl)-1,4,7,10-tetraazacyclododecane-1,4,7-triyl)triacetic acid

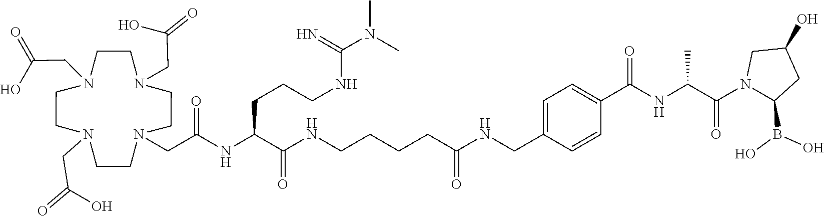 [SMILES: C[C@@H](NC(=O)c1ccc(CNC(=O)CCCCNC(=O)[C@H](CCCNC(=N)N(C)C)NC(=O)CN2CCN(CC(=O)O)CCN(CC(=O)O)CCN(CC(=O)O)CC2)cc1)C(=O)N1C[C@@H](O)C[C@H]1B(O)O]

(S,Z)-4-(2-Methyl-9,15-dioxo-3-(((2,2,4,6,7-pentamethyl-2,3-dihydrobenzofuran-5-yl)sulfonyl)imino)-8-(2-(4,7,10-tris(2-(tert-butoxy)-2-oxoethyl)-1,4,7,10-tetraazacyclododecan-1-yl)acetamido)-2,4,10,16-tetraazaheptadecan-17-yl)benzoic acid was coupled to (2R)-2-(l²-azaneyl)-1-((2R,4S)-4-hydroxy-2-((3aS,4S,6S)-3a,5,5-trimethylhexahydro-4,6-methanobenzo[d][1,3,2]dioxaborol-2-yl)pyrrolidin-1-yl)propan-1-one according to SP-5. The —Pbf, -tBu, and pinanediol protecting groups were removed according to SP-6 to afford the title compound (8.0 mg, 30% yield at 0.02 mmol scale) as a white powder. ESI/MS m/z: 988 (M−H₂O+H).

### Preparation 78

### 2,2′,2″-(10-(2-(((S)-1-((5-((4-(((R)-1-((2R,4S)-2-Borono-4-hydroxypyrrolidin-1-yl)-1-oxopropan-2-yl)carbamoyl)benzyl)(methyl)amino)-5-oxopentyl)(methyl)amino)-5-guanidino-1-oxopentan-2-yl)amino)-2-oxoethyl)-1,4,7,10-tetraazacyclododecane-1,4,7-triyl)triacetic acid

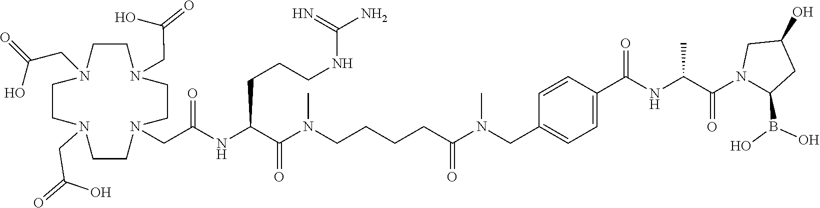 [SMILES: C[C@@H](NC(=O)c1ccc(CN(C)C(=O)CCCCN(C)C(=O)[C@H](CCCNC(=N)N)NC(=O)CN2CCN(CC(=O)O)CCN(CC(=O)O)CCN(CC(=O)O)CC2)cc1)C(=O)N1C[C@@H](O)C[C@H]1B(O)O]

4-((5-((S)-2-(l²-Azaneyl)-N-methyl-5-(3-((2,2,4,6,7-pentamethyl-2,3-dihydrobenzofuran-5-yl)sulfonyl)guanidino)pentanamido)-N-methylpentanamido)methyl)-N-((2R)-1-((2R,4S)-4-hydroxy-2-((3aR,4S,6R)-3a,5,5-trimethyloctahydro-2H-4,6-methanobenzo[c]borol-2-yl)pyrrolidin-1-yl)-1-oxopropan-2-yl)benzamide was coupled with 2,2′,2″-(10-(2-((2,5-dioxopyrrolidin-1-yl)oxy)-2-oxoethyl)-1,4,7,10-tetraazacyclododecane-1,4,7-triyl)triacetic acid (2.0 eq.) in anhydrous DMF with 10 eq. of DIPEA. The —Pbf and pinanediol protecting groups were removed according to SP-6 to afford the title compound (6.3 mg, 25% yield at 0.02 mmol scale) as a white powder. ESI/MS m/z: 988 (M−H₂O+H).

### Preparation 79

### 2,2′,2″-(10-(2-(((S)-1-((5-(4-(((R)-1-((2R,4S)-2-Borono-4-hydroxypyrrolidin-1-yl)-1-oxopropan-2-yl)carbamoyl)-1-naphthamido)pentyl)amino)-5-guanidino-1-oxopentan-2-yl)amino)-2-oxoethyl)-1,4,7,10-tetraazacyclododecane-1,4,7-triyl)triacetic acid

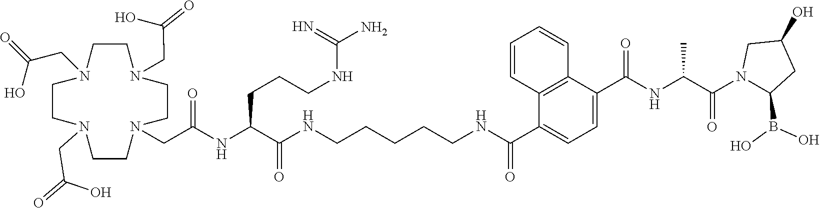 [SMILES: C[C@@H](NC(=O)c1ccc(C(=O)NCCCCCNC(=O)[C@H](CCCNC(=N)N)NC(=O)CN2CCN(CC(=O)O)CCN(CC(=O)O)CCN(CC(=O)O)CC2)c2ccccc12)C(=O)N1C[C@@H](O)C[C@H]1B(O)O]

N¹-(5-((S)-2-Amino-5-(3-((2,2-dimethyl-2,3-dihydrobenzofuran-5-yl)sulfonyl)guanidino)pentanamido)pentyl)-N⁴-((2R)-1-((2R,4S)-4-hydroxy-2-((3aS,4S,6S)-3a,5,5-trimethylhexahydro-4,6-methanobenzo[d][1,3,2]dioxaborol-2-yl)pyrrolidin-1-yl)-1-oxopropan-2-yl)naphthalene-1,4-dicarboxamide was coupled with 2,2′,2″-(10-(2-((2,5-dioxopyrrolidin-1-yl)oxy)-2-oxoethyl)-1,4,7,10-tetraazacyclododecane-1,4,7-triyl)triacetic acid (2.0 eq.) in anhydrous DMF with 10 eq. of DIPEA. The —Pbf and pinanediol protecting groups were removed according to SP-6 to afford the title compound (13.1 mg, 45% yield at 0.02 mmol scale) as a white powder. ESI/MS m/z: 1010 (M−H₂O+H).

### Preparation 80

### 2,2′,2″-(10-(2-(((S)-17-(4-(((R)-1-((2R,4S)-2-Borono-4-hydroxypyrrolidin-1-yl)-1-oxopropan-2-yl)carbamoyl)naphthalen-1-yl)-3-imino-2-methyl-9,17-dioxo-2,4,10,16-tetraazaheptadecan-8-yl)amino)-2-oxoethyl)-1,4,7,10-tetraazacyclododecane-1,4,7-triyl)triacetic acid

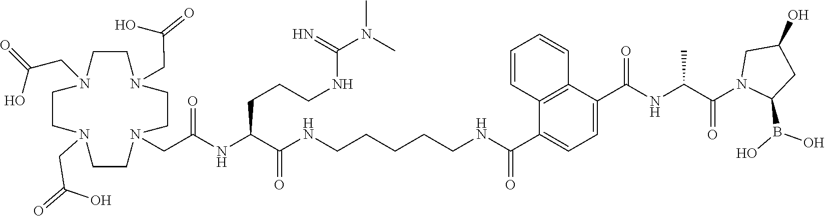 [SMILES: C[C@@H](NC(=O)c1ccc(C(=O)NCCCCCNC(=O)[C@H](CCCNC(=N)N(C)C)NC(=O)CN2CCN(CC(=O)O)CCN(CC(=O)O)CCN(CC(=O)O)CC2)c2ccccc12)C(=O)N1C[C@@H](O)C[C@H]1B(O)O]

The initial resin loading with naphthalene-1,4-dicarboxylic acid was performed as described in SP-1 and then coupled with (9H-fluoren-9-yl)methyl (5-aminopentyl)carbamate HCl according to SP-2 using PyBroP as the coupling reagent. The Fmoc group was removed according to SP-3 and then coupled with (Z)-N²-(((9H-fluoren-9-yl)methoxy)carbonyl)-N^{w′}-(dioxo(2,2,4,6,7-pentamethyl-2,3-dihydrobenzofuran-5-yl)-17-methyl)-N^{w},N^{w}-dimethyl-L-arginine according to SP-2 using PyBroP as the coupling reagent. The Fmoc group was removed according to SP-3 and then coupled with 2-(4,7,10-tris(2-(tert-butoxy)-2-oxoethyl)-1,4,7,10-tetraazacyclododecan-1-yl)acetic acid according to SP-2 using PyBroP as the coupling reagent. The compound was cleaved from the resin according to SP-4 and was coupled to (2R)-2-amino-1-((2R,4S)-4-hydroxy-2-((3aS,4S,6S)-3a,5,5-trimethylhexahydro-4,6-methanobenzo[d][1,3,2]dioxaborol-2-yl)pyrrolidin-1-yl)propan-1-one according to the SP-5. The —Pbf, -tBu and pinanediol protecting groups were removed according to SP-6 to give the title compound (6.0 mg, 4%) as a white powder. ESI/MS m/z: 1038 (M−H₂O+H).

### Preparation 81

### 2,2′,2″-(10-(2-(((S)-1-(4-(2-(4-(((R)-1-((2R,4S)-2-Borono-4-hydroxypyrrolidin-1-yl)-1-oxopropan-2-yl)carbamoyl)-1-naphthamido)ethyl)piperazin-1-yl)-5-(3,3-dimethylguanidino)-1-oxopentan-2-yl)amino)-2-oxoethyl)-1,4,7,10-tetraazacyclododecane-1,4,7-triyl)triacetic acid

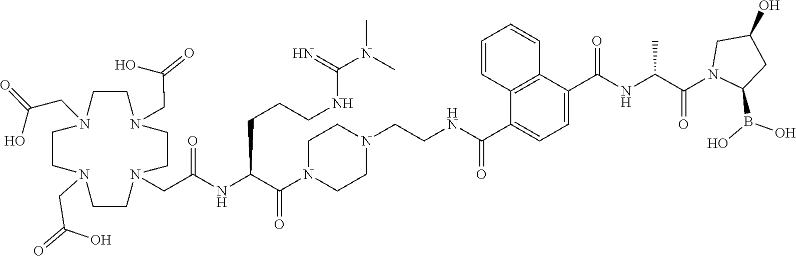 [SMILES: C[C@@H](NC(=O)c1ccc(C(=O)NCCN2CCN(C(=O)[C@H](CCCNC(=N)N(C)C)NC(=O)CN3CCN(CC(=O)O)CCN(CC(=O)O)CCN(CC(=O)O)CC3)CC2)c2ccccc12)C(=O)N1C[C@@H](O)C[C@H]1B(O)O]

4-((2-(4-(tert-Butoxycarbonyl)piperazin-1-yl)ethyl)carbamoyl)-1-naphthoic acid was coupled to (2R)-2-amino-1-((2R,4S)-4-hydroxy-2-((3aS,4S,6S)-3a,5,5-trimethylhexahydro-4,6-methanobenzo[d][1,3,2]dioxaborol-2-yl)pyrrolidin-1-yl)propan-1-one according to SP-5 and the Boc protecting group removed with 4N HCl in 1,4-dioxane. The free amine was coupled to (Z)-N²-(((9H-fluoren-9-yl)methoxy)carbonyl)-N^{w},N^{w}-dimethyl-N^{w′}-((2,2,4,6,7-pentamethyl-2,3-dihydrobenzofuran-5-yl)sulfonyl)-L-arginine according to SP-5 except using PyBroP instead of HATU as the coupling reagent. The Fmoc group was removed with 20% piperidine in DMF and was then coupled with 2,2′,2″-(10-(2-((2,5-dioxopyrrolidin-1-yl)oxy)-2-oxoethyl)-1,4,7,10-tetraazacyclododecane-1,4,7-triyl)triacetic acid (2.0 eq.) in anhydrous DMF with 10 eq. of DIPEA. The —Pbf and pinanediol protecting groups were removed according to SP-6 to give the title compound (18 mg, 26% yield at 0.05 mmol scale) as a white powder. ESI/MS m/z: 1065 (M−H₂O+H).

### Preparation 82

### 2,2′,2″-(10-(2-(((S)-17-(4-(((R)-1-((2R,4S)-2-Borono-4-hydroxypyrrolidin-1-yl)-1-oxopropan-2-yl)carbamoyl)naphthalen-1-yl)-3-imino-2,10-dimethyl-9,17-dioxo-2,4,10,16-tetraazaheptadecan-8-yl)amino)-2-oxoethyl)-1,4,7,10-tetraazacyclododecane-1,4,7-triyl)triacetic acid

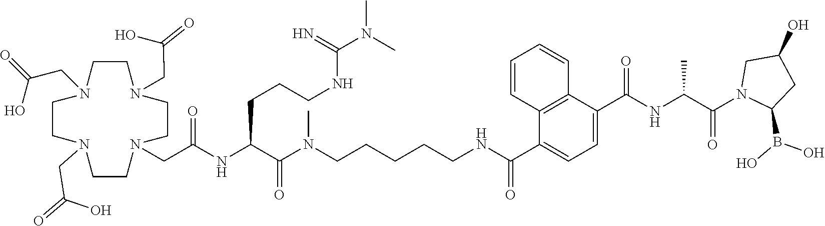 [SMILES: C[C@@H](NC(=O)c1ccc(C(=O)NCCCCCN(C)C(=O)[C@H](CCCNC(=N)N(C)C)NC(=O)CN2CCN(CC(=O)O)CCN(CC(=O)O)CCN(CC(=O)O)CC2)c2ccccc12)C(=O)N1C[C@@H](O)C[C@H]1B(O)O]

The initial resin loading with naphthalene-1,4-dicarboxylic acid was performed as described in SP-1 and then coupled with (9H-fluoren-9-yl)methyl (S,Z)-(1-((5-aminopentyl)(methyl)amino)-5-(3,3-dimethyl-2-((2,2,4,6,7-pentamethyl-2,3-dihydrobenzofuran-5-yl)sulfonyl)guanidino)-1-oxopentan-2-yl)carbamate according to SP-2 with PyBroP instead of HATU as the coupling reagent. The Fmoc group was removed according to SP-3 and was coupled with 2-(4,7,10-tris(2-(tert-butoxy)-2-oxoethyl)-1,4,7,10-tetraazacyclododecan-1-yl)acetic acid according to SP-2 with PyBroP instead of HATU as the coupling reagent. The compound was cleaved from the resin according to SP-4 and was coupled to (2R)-2-amino-1-((2R,4S)-4-hydroxy-2-((3aS,4S,6S)-3a,5,5-trimethylhexahydro-4,6-methanobenzo[d][1,3,2]dioxaborol-2-yl)pyrrolidin-1-yl)propan-1-one according to SP-5. The —Pbf, -tBu, and pinanediol protecting groups were removed according to SP-6 to afford the title compound (9.3 mg, 7%) as a white powder. ESI/MS m/z: 1052 (M−H₂O+H).

### Preparation 83

### 2,2′,2″-(10-((10S,13S)-1-(4-(((R)-1-((2R,4S)-2-Borono-4-hydroxypyrrolidin-1-yl)-1-oxopropan-2-yl)carbamoyl)naphthalen-1-yl)-10-(3-(3,3-dimethylguanidino)propyl)-13-hexanamido-1,9,12,19-tetraoxo-2,8,11,18-tetraazaicosan-20-yl)-1,4,7,10-tetraazacyclododecane-1,4,7-triyl)triacetic acid

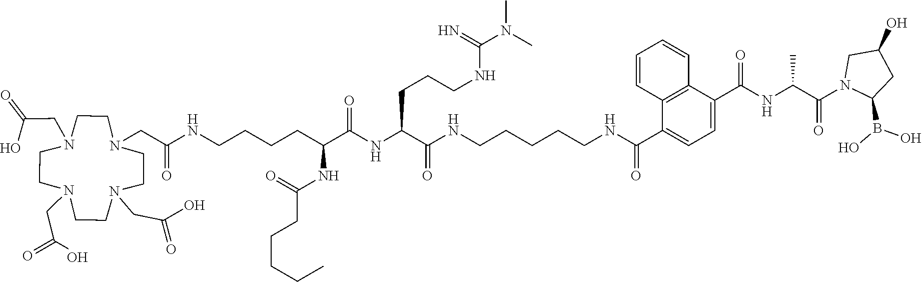 [SMILES: CCCCCC(=O)N[C@@H](CCCCNC(=O)CN1CCN(CC(=O)O)CCN(CC(=O)O)CCN(CC(=O)O)CC1)C(=O)N[C@@H](CCCNC(=N)N(C)C)C(=O)NCCCCCNC(=O)c1ccc(C(=O)N[C@H](C)C(=O)N2C[C@@H](O)C[C@H]2B(O)O)c2ccccc12]

4-((5-((S)-5-((Z)-3,3-Dimethyl-2-((2,2,4,6,7-pentamethyl-2,3-dihydrobenzofuran-5-yl)sulfonyl)guanidino)-2-((S)-2-hexanamido-6-(2-(4,7,10-tris(2-(tert-butoxy)-2-oxoethyl)-1,4,7,10-tetraazacyclododecan-1-yl)acetamido)hexanamido)pentanamido)pentyl)carbamoyl)-1-naphthoic acid was coupled to (2R)-2-amino-1-((2R,4S)-4-hydroxy-2-((3aS,4S,6S)-3a,5,5-trimethylhexahydro-4,6-methanobenzo[d][1,3,2]dioxaborol-2-yl)pyrrolidin-1-yl)propan-1-one according to SP-5. The —Pbf, -tBu, and pinanediol protecting groups were removed according to SP-6 to afford the title compound (26.6 mg, 32% yield at 0.05 mmol scale) as a white powder. ESI/MS m/z: 1264 (M−H₂O+H).

### Preparation 84

### 2,2′,2″-(10-((10S,13S)-1-(4-(((R)-1-((2R,4S)-2-Borono-4-hydroxypyrrolidin-1-yl)-1-oxopropan-2-yl)carbamoyl)naphthalen-1-yl)-10-(3-(3,3-dimethylguanidino)propyl)-13-(4-((4-(4-iodophenyl)butanamido)methyl)benzamido)-1,9,12,19-tetraoxo-2,8,11,18-tetraazaicosan-20-yl)-1,4,7,10-tetraazacyclododecane-1,4,7-triyl)triacetic acid

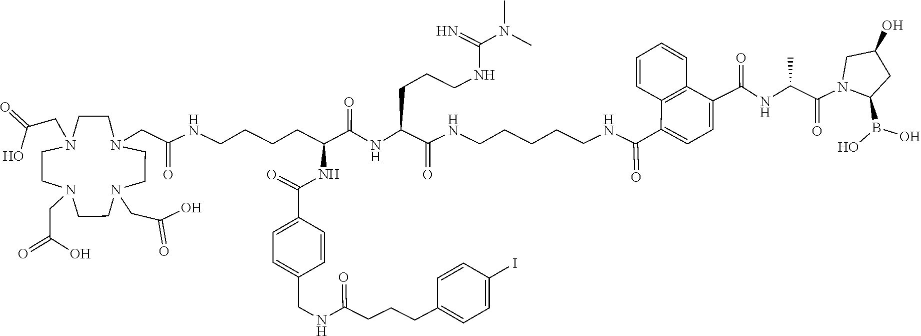 [SMILES: C[C@@H](NC(=O)c1ccc(C(=O)NCCCCCNC(=O)[C@H](CCCNC(=N)N(C)C)NC(=O)[C@H](CCCCNC(=O)CN2CCN(CC(=O)O)CCN(CC(=O)O)CCN(CC(=O)O)CC2)NC(=O)c2ccc(CNC(=O)CCCc3ccc(I)cc3)cc2)c2ccccc12)C(=O)N1C[C@@H](O)C[C@H]1B(O)O]

4-((5-((S)-5-((Z)-3,3-Dimethyl-2-((2,2,4,6,7-pentamethyl-2,3-dihydrobenzofuran-5-yl)sulfonyl)guanidino)-2-((S)-2-(4-((4-(4-iodophenyl)butanamido)methyl)benzamido)-6-(2-(4,7,10-tris(2-(tert-butoxy)-2-oxoethyl)-1,4,7,10-tetraazacyclododecan-1-yl)acetamido)hexanamido)pentanamido)pentyl)carbamoyl)-1-naphthoic acid was coupled to (2R)-2-amino-1-((2R,4S)-4-hydroxy-2-((3aS,4S,6S)-3a,5,5-trimethylhexahydro-4,6-methanobenzo[d][1,3,2]dioxaborol-2-yl)pyrrolidin-1-yl)propan-1-one according to the SP-5. The —Pbf, -tBu, and pinanediol protective groups were removed according to SP-6 to afford the title compound (21.4 mg, 22% yield at 0.05 mmol scale) as a white powder. ESI/MS m/z: 1571 (M−H₂O+H).

### Preparation 85

### 2,2′,2″-(10-((10S,13S)-1-(4-(((R)-1-((2R,4S)-2-Borono-4-hydroxypyrrolidin-1-yl)-1-oxopropan-2-yl)carbamoyl)naphthalen-1-yl)-10-(3-(3,3-dimethylguanidino)propyl)-13-(4-(4-iodophenyl)butanamido)-1,9,12,19-tetraoxo-2,8,11,18-tetraazaicosan-20-yl)-1,4,7,10-tetraazacyclododecane-1,4,7-triyl)triacetic acid

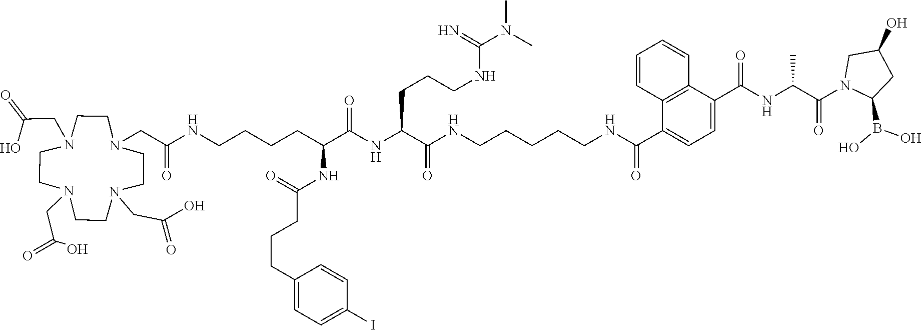 [SMILES: C[C@@H](NC(=O)c1ccc(C(=O)NCCCCCNC(=O)[C@H](CCCNC(=N)N(C)C)NC(=O)[C@H](CCCCNC(=O)CN2CCN(CC(=O)O)CCN(CC(=O)O)CCN(CC(=O)O)CC2)NC(=O)CCCc2ccc(I)cc2)c2ccccc12)C(=O)N1C[C@@H](O)C[C@H]1B(O)O]

4-((5-((S)-5-((Z)-3,3-Dimethyl-2-((2,2,4,6,7-pentamethyl-2,3-dihydrobenzofuran-5-yl)sulfonyl)guanidino)-2-((S)-2-(4-(4-iodophenyl)butanamido)-6-(2-(4,7,10-tris(2-(tert-butoxy)-2-oxoethyl)-1,4,7,10-tetraazacyclododecan-1-yl)acetamido)hexanamido)pentanamido)pentyl)carbamoyl)-1-naphthoic acid was coupled to (2R)-2-amino-1-((2R,4S)-4-hydroxy-2-((3aS,4S,6S)-3a,5,5-trimethylhexahydro-4,6-methanobenzo[d][1,3,2]dioxaborol-2-yl)pyrrolidin-1-yl)propan-1-one according to SP-5. The —Pbf, -tBu, and pinanediol protecting groups were removed according to SP-6 to give the title compound (27.5 mg, 30% yield at 0.05 mmol scale) as a white powder. ESI/MS m/z: 1438 (M−H₂O+H).

### Preparation 86

### 2,2′,2″-(10-((10S,13S)-1-(4-(((R)-1-((2R,4S)-2-Borono-4-hydroxypyrrolidin-1-yl)-1-oxopropan-2-yl)carbamoyl)naphthalen-1-yl)-13-((R)-3-carboxy-2-(4-(4-iodophenyl)butanamido)propanamido)-10-(3-(3,3-dimethylguanidino)propyl)-1,9,12,19-tetraoxo-2,8,11,18-tetraazaicosan-20-yl)-1,4,7,10-tetraazacyclododecane-1,4,7-triyl)triacetic acid

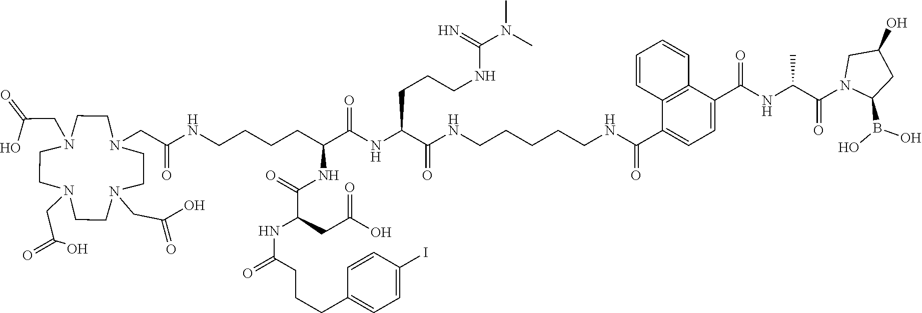 [SMILES: C[C@@H](NC(=O)c1ccc(C(=O)NCCCCCNC(=O)[C@H](CCCNC(=N)N(C)C)NC(=O)[C@H](CCCCNC(=O)CN2CCN(CC(=O)O)CCN(CC(=O)O)CCN(CC(=O)O)CC2)NC(=O)[C@@H](CC(=O)O)NC(=O)CCCc2ccc(I)cc2)c2ccccc12)C(=O)N1C[C@@H](O)C[C@H]1B(O)O]

4-(((6R,9S,12S)-12-(3-((Z)-3,3-Dimethyl-2-(2-(2,2,4,6,7-pentamethyl-2,3-dihydrobenzofuran-5-yl)-217-propa-1,2-dien-2-yl)guanidino)propyl)-6-(4-(4-iodophenyl)butanamido)-2,2-dimethyl-4,7,10,13-tetraoxo-9-(4-(2-(4,7,10-tris(2-(tert-butoxy)-2-oxoethyl)-1,4,7,10-tetraazacyclododecan-1-yl)acetamido)butyl)-3-oxa-8,11,14-triazanonadecan-19-yl)carbamoyl)-1-naphthoic acid was coupled to (2R)-2-amino-1-((2R,4S)-4-hydroxy-2-((3aS,4S,6S)-3a,5,5-trimethylhexahydro-4,6-methanobenzo[d][1,3,2]dioxaborol-2-yl)pyrrolidin-1-yl)propan-1-one according to SP-5. The —Pbf, -tBu and pinanediol protecting groups were removed according to SP-6 to afford the title compound (19.7 mg, 20% yield at 0.05 mmol scale) as a white powder. ESI/MS m/z: 1553 (M−H₂O+H).

### Preparation 87

### 2,2′,2″-(10-((10S,20S)-1-(4-(((R)-1-((2R,4S)-2-Borono-4-hydroxypyrrolidin-1-yl)-1-oxopropan-2-yl)carbamoyl)naphthalen-1-yl)-20-(((S)-17-(4-(((R)-1-((2R,4S)-2-borono-4-hydroxypyrrolidin-1-yl)-1-oxopropan-2-yl)carbamoyl)naphthalen-1-yl)-3-imino-2-methyl-9,17-dioxo-2,4,10,16-tetraazaheptadecan-8-yl)carbamoyl)-10-(3-(3,3-dimethylguanidino)propyl)-1,9,12,18,26-pentaoxo-2,8,11,19,25-pentaazaheptacosan-27-yl)-1,4,7,10-tetraazacyclododecane-1,4,7-triyl)triacetic acid

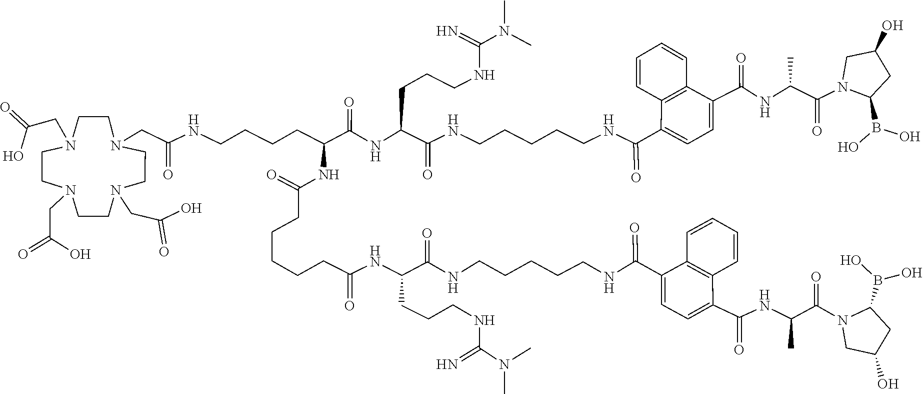 [SMILES: C[C@@H](NC(=O)c1ccc(C(=O)NCCCCCNC(=O)[C@H](CCCNC(=N)N(C)C)NC(=O)CCCCCC(=O)N[C@@H](CCCCNC(=O)CN2CCN(CC(=O)O)CCN(CC(=O)O)CCN(CC(=O)O)CC2)C(=O)N[C@@H](CCCNC(=N)N(C)C)C(=O)NCCCCCNC(=O)c2ccc(C(=O)N[C@H](C)C(=O)N3C[C@@H](O)C[C@H]3B(O)O)c3ccccc23)c2ccccc12)C(=O)N1C[C@@H](O)C[C@H]1B(O)O]

The initial resin loading with naphthalene-1,4-dicarboxylic acid was performed as described in SP-1 and then coupled with (9H-fluoren-9-yl)methyl (5-aminopentyl)carbamate HCl according to SP-2 using PyBroP instead of HATU as the coupling reagent. The Fmoc group was removed according to SP-3 and then coupled with (Z)-N²-(((9H-fluoren-9-yl)methoxy)carbonyl)-N^{w},N^{w}-dimethyl-N^{w′}-((2,2,4,6,7-pentamethyl-2,3-dihydrobenzofuran-5-yl)sulfonyl)-L-arginine according to SP-2 using PyBroP instead of HATU as the coupling reagent. The Fmoc group was removed according to SP-3 and then coupled to N²-(((9H-fluoren-9-yl)methoxy)carbonyl)-N⁶-(tert-butoxycarbonyl)-L-lysine according to SP-2 using TBTU/HOBt instead of HATU as the coupling reagents. The compound was cleaved from the resin according to SP-4 and then coupled to (2R)-2-amino-1-((2R,4S)-4-hydroxy-2-((3aS,4S,6S)-3a,5,5-trimethylhexahydro-4,6-methanobenzo[d][1,3,2]dioxaborol-2-yl)pyrrolidin-1-yl)propan-1-one according to SP-5. The Fmoc on the Lys moiety was removed with 20% piperidine in DMF and then coupled to 7-(((8S,Z)-17-(4-(((2R)-1-((2R,4S)-4-hydroxy-2-((3aS,4S,6S)-3a,5,5-trimethylhexahydro-4,6-methanobenzo[d][1,3,2]dioxaborol-2-yl)pyrrolidin-1-yl)-1-oxopropan-2-yl)carbamoyl)naphthalen-1-yl)-2-methyl-9,17-dioxo-3-(((2,2,4,6,7-pentamethyl-2,3-dihydrobenzofuran-5-yl)sulfonyl)imino)-2,4,10,16-tetraazaheptadecan-8-yl)amino)-7-oxoheptanoic acid according to SP-5 except using PyBroP instead of HATU as the coupling reagent. The Boc on the Lys moiety was removed with 4N HCl in 1,4-dioxane and then coupled with 2,2′,2″-(10-(2-((2,5-dioxopyrrolidin-1-yl)oxy)-2-oxoethyl)-1,4,7,10-tetraazacyclododecane-1,4,7-triyl)triacetic acid (2.0 eq.) in anhydrous DMF with 10 eq. of DIPEA. The —Pbf and pinanediol protecting groups were removed according to SP-6 to give the title compound (7.6 mg, 3%) as a white powder. ESI/MS m/z: 971 (M/2-H₂O+H).

### Preparation 88

### 2,2′,2″-(10-(2-(((S)-17-((1R,4S)-4-(((R)-1-((2R,4S)-2-Borono-4-hydroxypyrrolidin-1-yl)-1-oxopropan-2-yl)carbamoyl)cyclohexyl)-3-imino-2,10-dimethyl-9,15-dioxo-2,4,10,16-tetraazaheptadecan-8-yl)amino)-2-oxoethyl)-1,4,7,10-tetraazacyclododecane-1,4,7-triyl)triacetic acid

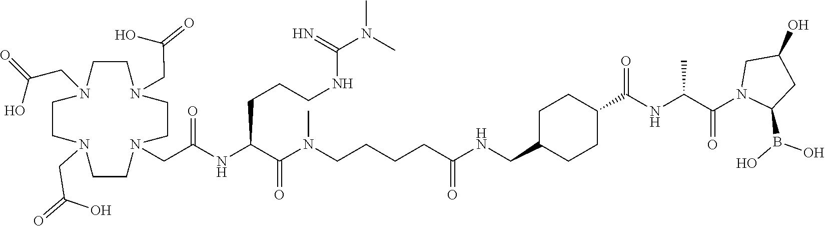

(1S,4r)-4-((S,Z)-2,10-Dimethyl-9,15-dioxo-3-(((2,2,4,6,7-pentamethyl-2,3-dihydrobenzofuran-5-yl)sulfonyl)imino)-8-(2-(4,7,10-tris(2-(tert-butoxy)-2-oxoethyl)-1,4,7,10-tetraazacyclododecan-1-yl)acetamido)-2,4,10,16-tetraazaheptadecan-17-yl)cyclohexane-1-carboxylic acid was coupled with (2R)-2-amino-1-((2R,4S)-4-hydroxy-2-((3aS,4S,6S)-3a,5,5-trimethylhexahydro-4,6-methanobenzo[d][1,3,2]dioxaborol-2-yl)pyrrolidin-1-yl)propan-1-one HCl according to SP-5 and then all remaining protecting groups (—Pbf, -tBu, and pinanediol) were removed according to SP-6 to afford the title compound (16.0 mg, 58% yield at 0.02 mmol scale). ESI/MS m/z: 1008 (M−H₂O+H).

### Preparation 89

### 2,2′,2″-(10-((10S,20S)-1-((1R,4S)-4-(((R)-1-((2R,4S)-2-Borono-4-hydroxypyrrolidin-1-yl)-1-oxopropan-2-yl)carbamoyl)cyclohexyl)-20-(((S)-17-((1R,4S)-4-(((R)-1-((2R,4S)-2-borono-4-hydroxypyrrolidin-1-yl)-1-oxopropan-2-yl)carbamoyl)cyclohexyl)-3-imino-2-methyl-9,15-dioxo-2,4,10,16-tetraazaheptadecan-8-yl)carbamoyl)-10-(3-(3,3-dimethylguanidino)propyl)-3,9,12,18,26-pentaoxo-2,8,11,19,25-pentaazaheptacosan-27-yl)-1,4,7,10-tetraazacyclododecane-1,4,7-triyl)triacetic acid

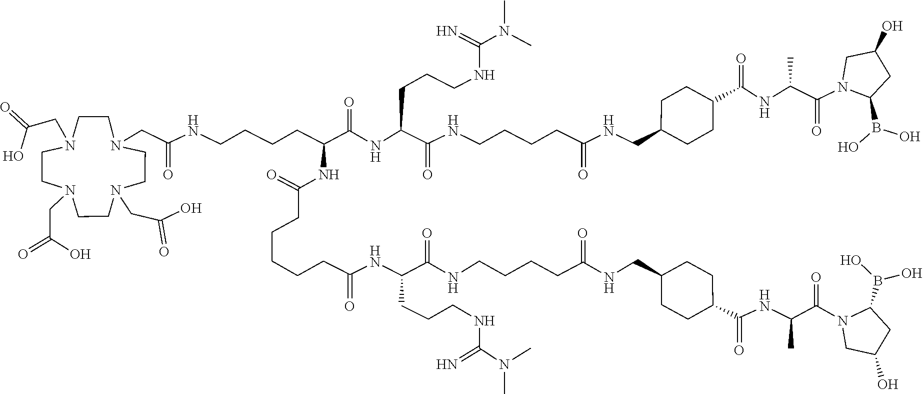

The initial resin loading with trans-4-(((((9H-fluoren-9-yl)methoxy)carbonyl)amino)methyl)cyclohexane-1-carboxylic acid was performed according to SP-1 and coupled with N²-(((9H-fluoren-9-yl)methoxy)carbonyl)-N⁶-(1-(4,4-dimethyl-2,6-dioxocyclohexylidene)ethyl)-L-lysine according to SP-2. The -Dde protecting group was removed by a mixture of NH₂OH HCl/imidazole/NMP in DMF-DCM and then coupled with 2-(4,7,10-tris(2-(tert-butoxy)-2-oxoethyl)-1,4,7,10-tetraazacyclododecan-1-yl)acetic acid according to SP-2 using PyBroP instead of HATU. The Fmoc on the Lys moiety was removed according to SP-3 and then coupled to the free carboxylic acid on 7-(4-nitrophenoxy)-7-oxoheptanoic acid according to SP-2 using PyBroP as the coupling reagent. The PNP ester was then coupled with (1S,4r)-4-((S,Z)-8-(l²-azaneyl)-2-methyl-9,15-dioxo-3-(((2,2,4,6,7-pentamethyl-2,3-dihydrobenzofuran-5-yl)sulfonyl)imino)-2,4,10,16-tetraazaheptadecan-17-yl)cyclohexane-1-carboxylic acid in DMF with the addition of DIPEA. The compound was cleaved from the resin according to SP-4 and coupled with (2R)-2-amino-1-((2R,4S)-4-hydroxy-2-((3aS,4S,6S)-3a,5,5-trimethylhexahydro-4,6-methanobenzo[d][1,3,2]dioxaborol-2-yl)pyrrolidin-1-yl)propan-1-one HCl according to SP-5. The remaining protecting groups (—Pbf, -tBu, and pinanediol) were removed according to SP-6 to afford the title compound (11.2 mg, 5%) as a white powder. ESI/MS m/z: 927 (M/2-H₂O+H).

### Preparation 90

### 2,2′,2″-(10-((10S,29R)-1-(4-(((R)-1-((2R,4S)-2-Borono-4-hydroxypyrrolidin-1-yl)-1-oxopropan-2-yl)carbamoyl)naphthalen-1-yl)-29-carboxy-10-(3-(3,3-dimethylguanidino)propyl)-1,9,12,18,26-pentaoxo-2,8,11,19,25-pentaazanonacosan-29-yl)-1,4,7,10-tetraazacyclododecane-1,4,7-triyl)triacetic acid

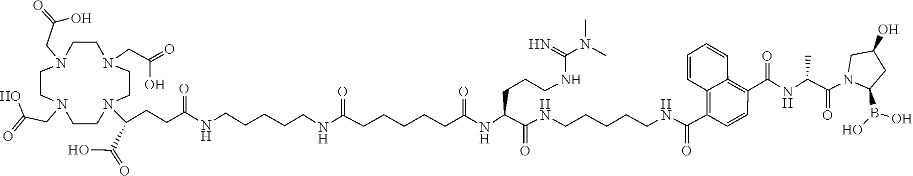 [SMILES: C[C@@H](NC(=O)c1ccc(C(=O)NCCCCCNC(=O)[C@H](CCCNC(=N)N(C)C)NC(=O)CCCCCC(=O)NCCCCCNC(=O)CC[C@H](C(=O)O)N2CCN(CC(=O)O)CCN(CC(=O)O)CCN(CC(=O)O)CC2)c2ccccc12)C(=O)N1C[C@@H](O)C[C@H]1B(O)O]

(S,Z)-4-((20-(3-(3,3-Dimethyl-2-((2,2,4,6,7-pentamethyl-2,3-dihydrobenzofuran-5-yl)sulfonyl)guanidino)propyl)-2,2-dimethyl-4,12,18,21-tetraoxo-3-oxa-5,11,19,22-tetraazaheptacosan-27-yl)carbamoyl)-1-naphthoic acid was coupled to (2R)-2-amino-1-((2R,4S)-4-hydroxy-2-((3aS,4S,6S)-3a,5,5-trimethylhexahydro-4,6-methanobenzo[d][1,3,2]dioxaborol-2-yl)pyrrolidin-1-yl)propan-1-one HCl according to SP-5. After removal of the Boc protecting group with 4N HCl in 1,4-dioxane, the free amine was then coupled to (R)-2,2′,2″-(10-(1-carboxy-4-(4-nitrophenoxy)-4-oxobutyl)-1,4,7,10-tetraazacyclododecane-1,4,7-triyl)triacetic acid (2.0 eq.) in anhydrous DMF with 10 eq. of DIPEA. The remaining protecting groups were removed according to SP-6 to give the title compound (9.0 mg, 25% yield at 0.02 mmol scale) as a white powder. ESI/MS m/z: 1318 (M-2H₂O+H).

### Preparation 91

### 2,2′,2″-(10-((S)-1-(4-(((R)-1-((2R,4S)-2-Borono-4-hydroxypyrrolidin-1-yl)-1-oxopropan-2-yl)carbamoyl)naphthalen-1-yl)-10-(3-(3,3-dimethylguanidino)propyl)-1,9,12,18,26-pentaoxo-2,8,11,19,25-pentaazaheptacosan-27-yl)-1,4,7,10-tetraazacyclododecane-1,4,7-triyl)triacetic acid

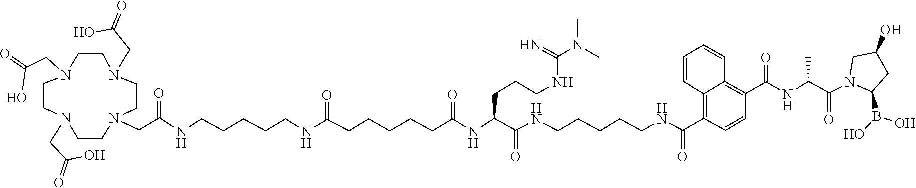 [SMILES: C[C@@H](NC(=O)c1ccc(C(=O)NCCCCCNC(=O)[C@H](CCCNC(=N)N(C)C)NC(=O)CCCCCC(=O)NCCCCCNC(=O)CN2CCN(CC(=O)O)CCN(CC(=O)O)CCN(CC(=O)O)CC2)c2ccccc12)C(=O)N1C[C@@H](O)C[C@H]1B(O)O]

(S,Z)-4-((18-(3-(3,3-Dimethyl-2-((2,2,4,6,7-pentamethyl-2,3-dihydrobenzofuran-5-yl)sulfonyl)guanidino)propyl)-2,10,16,19-tetraoxo-1-(4,7,10-tris(2-(tert-butoxy)-2-oxoethyl)-1,4,7,10-tetraazacyclododecan-1-yl)-3,9,17,20-tetraazapentacosan-25-yl)carbamoyl)-1-naphthoic acid was coupled to (2R)-2-amino-1-((2R,4S)-4-hydroxy-2-((3aS,4S,6S)-3a,5,5-trimethylhexahydro-4,6-methanobenzo[d][1,3,2]dioxaborol-2-yl)pyrrolidin-1-yl)propan-1-one HCl according to SP-5. All protecting groups were removed according to SP-6 to give the title compound (9.8 mg, 30% yield at 0.02 mmol scale) as a white powder. ESI/MS m/z: 1264 (M−H₂O+H).

### Preparation 92

### 2,2′,2″-(10-((10S,29R)-1-(4-(((R)-1-((2R,4S)-2-Borono-4-hydroxypyrrolidin-1-yl)-1-oxopropan-2-yl)carbamoyl)phenyl)-29-carboxy-10-(3-(3,3-dimethylguanidino)propyl)-3,9,12,18,26-pentaoxo-2,8,11,19,25-pentaazanonacosan-29-yl)-1,4,7,10-tetraazacyclododecane-1,4,7-triyl)triacetic acid

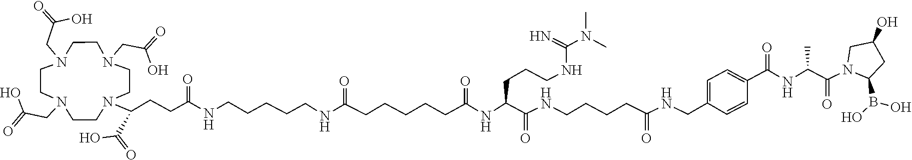 [SMILES: C[C@@H](NC(=O)c1ccc(CNC(=O)CCCCNC(=O)[C@H](CCCNC(=N)N(C)C)NC(=O)CCCCCC(=O)NCCCCCNC(=O)CC[C@H](C(=O)O)N2CCN(CC(=O)O)CCN(CC(=O)O)CCN(CC(=O)O)CC2)cc1)C(=O)N1C[C@@H](O)C[C@H]1B(O)O]

(S,Z)-4-(10-(3-(3,3-Dimethyl-2-((2,2,4,6,7-pentamethyl-2,3-dihydrobenzofuran-5-yl)sulfonyl)guanidino)propyl)-28,28-dimethyl-3,9,12,18,26-pentaoxo-27-oxa-2,8,11,19,25-pentaazanonacosyl)benzoic acid was coupled to (2R)-2-amino-1-((2R,4S)-4-hydroxy-2-((3aS,4S,6S)-3a,5,5-trimethylhexahydro-4,6-methanobenzo[d][1,3,2]dioxaborol-2-yl)pyrrolidin-1-yl)propan-1-one HCl according to SP-5. After removal of the Boc protecting group with 4N HCl in 1,4-dioxane, the free amine was then coupled with (R)-2,2′,2″-(10-(1-carboxy-4-(4-nitrophenoxy)-4-oxobutyl)-1,4,7,10-tetraazacyclododecane-1,4,7-triyl)triacetic acid (2.0 eq.) in anhydrous DMF with 10 eq. of DIPEA. The remaining protecting groups were removed according to SP-6 to give the title compound (6.7 mg, 20% yield at 0.02 mmol scale) as a white powder. ESI/MS m/z: 1286 (M−H₂O+H).

### Preparation 93

### 2,2′,2″-(10-(2-(((S)-1-((5-((4-(((R)-1-((2R,4S)-2-Borono-4-hydroxypyrrolidin-1-yl)-1-oxopropan-2-yl)carbamoyl)quinolin-8-yl)amino)-5-oxopentyl)amino)-5-guanidino-1-oxopentan-2-yl)amino)-2-oxoethyl)-1,4,7,10-tetraazacyclododecane-1,4,7-triyl)triacetic acid

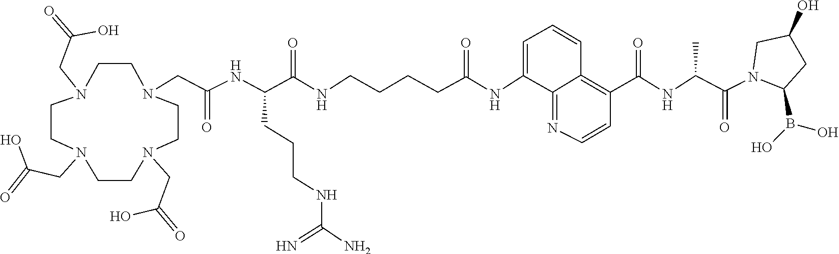 [SMILES: C[C@@H](NC(=O)c1ccnc2c(NC(=O)CCCCNC(=O)[C@H](CCCNC(=N)N)NC(=O)CN3CCN(CC(=O)O)CCN(CC(=O)O)CCN(CC(=O)O)CC3)cccc12)C(=O)N1C[C@@H](O)C[C@H]1B(O)O]

8-(5-((((9H-Fluoren-9-yl)methoxy)carbonyl)amino)pentanamido)quinoline-4-carboxylic acid was coupled to (2R)-2-amino-1-((2R,4S)-4-hydroxy-2-((3aS,4S,6S)-3a,5,5-trimethylhexahydro-4,6-methanobenzo[d][1,3,2]dioxaborol-2-yl)pyrrolidin-1-yl)propan-1-one HCl according to SP-5. The Fmoc group was removed according to SP-3 and then coupled with N²-(((9H-fluoren-9-yl)methoxy)carbonyl)-N^{w}-((2,2,4,6,7-pentamethyl-2,3-dihydrobenzofuran-5-yl)sulfonyl)-L-arginine according to SP-2 with PyBroP instead of HATU as the coupling reagent. The Fmoc group was removed according to SP-3 and then coupled with 2,2′,2″-(10-(2-((2,5-dioxopyrrolidin-1-yl)oxy)-2-oxoethyl)-1,4,7,10-tetraazacyclododecane-1,4,7-triyl)triacetic acid in DMF with TEA. All the protecting groups were removed according to SP-6 to give the title compound (19 mg, 14% yield at 0.1 mmol scale) as a white powder. ESI/MS m/z: 996 (M−H₂O+H).

### Preparation 94

### 2,2′,2″-(10-(2-(((S)-1-((2-(((S)-1-((4-(((R)-1-((2R,4S)-2-Borono-4-hydroxypyrrolidin-1-yl)-1-oxopropan-2-yl)carbamoyl)quinolin-8-yl)amino)-3-carboxy-1-oxopropan-2-yl)amino)-2-oxoethyl)amino)-5-guanidino-1-oxopentan-2-yl)amino)-2-oxoethyl)-1,4,7,10-tetraazacyclododecane-1,4,7-triyl)triacetic acid

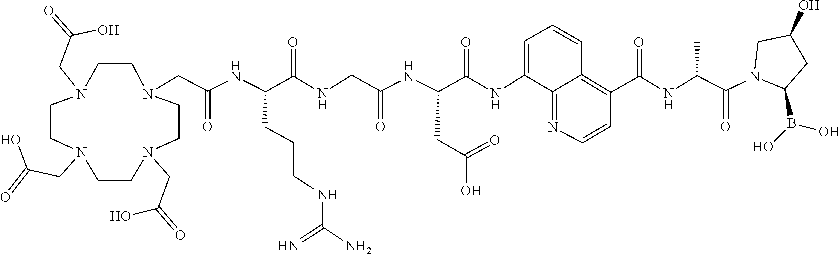 [SMILES: C[C@@H](NC(=O)c1ccnc2c(NC(=O)[C@H](CC(=O)O)NC(=O)CNC(=O)[C@H](CCCNC(=N)N)NC(=O)CN3CCN(CC(=O)O)CCN(CC(=O)O)CCN(CC(=O)O)CC3)cccc12)C(=O)N1C[C@@H](O)C[C@H]1B(O)O]

8-((5S,11S)-11-(2-(tert-Butoxy)-2-oxoethyl)-1-(9H-fluoren-9-yl)-3,6,9-trioxo-5-(3-(3-((2,2,4,6,7-pentamethyl-2,3-dihydrobenzofuran-5-yl)sulfonyl)guanidino)propyl)-2-oxa-4,7,10-triazadodecan-12-amido)quinoline-4-carboxylic acid was coupled to (2R)-2-amino-1-((2R,4S)-4-hydroxy-2-((3aS,4S,6S)-3a,5,5-trimethylhexahydro-4,6-methanobenzo[d][1,3,2]dioxaborol-2-yl)pyrrolidin-1-yl)propan-1-one HCl according to SP-5. The Fmoc group was removed according to SP-3 and then was coupled with 2,2′,2″-(10-(2-((2,5-dioxopyrrolidin-1-yl)oxy)-2-oxoethyl)-1,4,7,10-tetraazacyclododecane-1,4,7-triyl)triacetic acid in DMF with TEA. All the protecting groups were removed according to SP-6 to give the title compound (5.3 mg, 18% yield at 0.02 mmol scale) as a white powder. ESI/MS m/z: 1069 (M−H₂O+H).

### Preparation 100

### 2,2′,2″-(10-((10S,20S)-1-(4-(((R)-1-((2R,4S)-2-Borono-4-hydroxypyrrolidin-1-yl)-1-oxopropan-2-yl)carbamoyl)naphthalen-1-yl)-20-(((R)-1-carboxy-5-(4-(4-iodophenyl)butanamido)pentyl)carbamoyl)-10-(3-(3,3-dimethylguanidino)propyl)-1,9,12,18,26-pentaoxo-2,8,11,19,25-pentaazaheptacosan-27-yl)-1,4,7,10-tetraazacyclododecane-1,4,7-triyl)triacetic acid

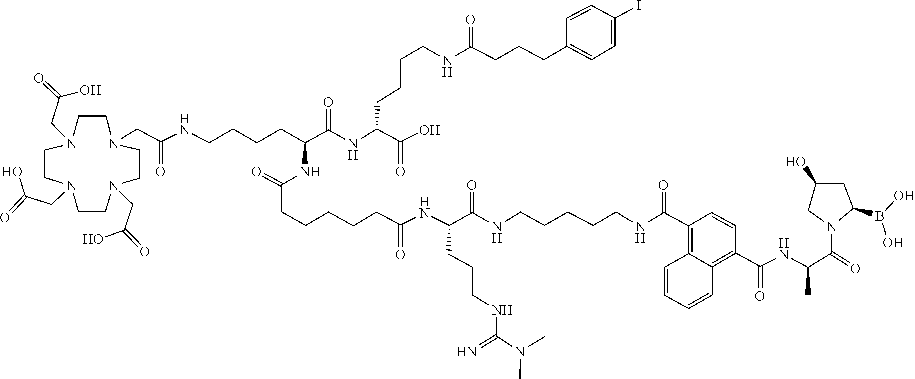 [SMILES: C[C@@H](NC(=O)c1ccc(C(=O)NCCCCCNC(=O)[C@H](CCCNC(=N)N(C)C)NC(=O)CCCCCC(=O)N[C@@H](CCCCNC(=O)CN2CCN(CC(=O)O)CCN(CC(=O)O)CCN(CC(=O)O)CC2)C(=O)N[C@H](CCCCNC(=O)CCCc2ccc(I)cc2)C(=O)O)c2ccccc12)C(=O)N1C[C@@H](O)C[C@H]1B(O)O]

The initial resin loading with N2-(((9H-fluoren-9-yl)methoxy)carbonyl)-N6-(4-(4-iodophenyl)butanoyl)-D-lysine was performed according to SP-1. The Fmoc was removed according to SP-3 and was then coupled with N2-(((9H-fluoren-9-yl)methoxy)carbonyl)-N6-(1-(4,4-dimethyl-2,6-dioxocyclohexylidene)ethyl)-L-lysine according to the SP-2 except using PyBrop instead of HATU. The -Dde protecting group was removed with imidazole/NH₂OH·HCl in DMF and DCM with the addition of NMP and was then coupled with 2-(4,7,10-tris(2-(tert-butoxy)-2-oxoethyl)-1,4,7,10-tetraazacyclododecan-1-yl)acetic acid according to SP-2 except using PyBrop instead of HATU. The Fmoc was removed according to the SP-3 and was then coupled with 7-(((8S,Z)-17-(4-(((2R)-1-((2R,4S)-4-hydroxy-2-((3aS,4S,6S)-3a,5,5-trimethylhexahydro-4,6-methanobenzo[d][1,3,2]dioxaborol-2-yl)pyrrolidin-1-yl)-1-oxopropan-2-yl)carbamoyl)naphthalen-1-yl)-2-methyl-9,17-dioxo-3-((2,2,4,6,7-pentamethyl-2,3-dihydrobenzofuran)-5-sulfonamido)-2,4,10,16-tetraazaheptadec-3-en-8-yl)amino)-7-oxoheptanoic acid according to the SP-2 except using PyBrop instead of HATU. The peptide was cleaved from the resin and all the protecting groups removed with TFA-TIPS-H₂O according to SP-6 to give the title compound (5.3 mg, 5% yield at 0.05 mmol scale). ESI/MS (m/z): 1708 (M−H2O+H).

### Preparation 101

### 4-(5-((tert-Butoxycarbonyl)amino)pentanamido)-1-naphthoic acid

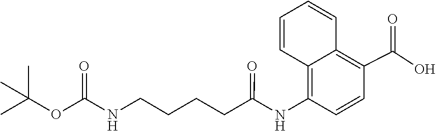 [SMILES: CC(C)(C)OC(=O)NCCCCC(=O)Nc1ccc(C(=O)O)c2ccccc12]

A stirred solution of 5-((((9H-fluoren-9-yl)methoxy)carbonyl)amino)pentanoic acid (2.3 g, 6.8 mmol) in anhydrous THF (50 mL) was treated with anhydrous DMF (15 μL) and oxalyl chloride (3.8 mL) dropwise under ice-water bath. The resulting mixture was stirred at rt for 2 h and then concentrated under reduced pressure. The residue was dissolved into anhydrous pyridine (25 mL). DMAP (20 mg) was added and then methyl 4-aminonaphthalene-1-carboxylate (0.33 g, 1.64 mmol) was added. The reaction was stirred at 60° C. for 4 h and then at rt overnight. The mixture was concentrated under reduced pressure and piperidine (6%) in DMF (25 mL) was added. After concentration at reduced pressure, the residue was purified by preparative HPLC as described in the general procedure. The residue was dissolved in DCM (20 mL) and TEA (1 mL) and Boc₂O (0.8 g) were added. The resulting mixture was stirred at rt overnight and then washed with 0.1N KHSO₄, aqueous NaHCO₃, and saturated aqueous NaCl solution. The solution was concentrated and hydrolyzed with 1N LiOH (3 mL), 5N NaOH (1 mL) in H₂O (7 mL), MeOH (10 mL) and THF (10 mL) at rt overnight. The reaction pH was adjusted to 3 with 1N aqueous HCl and then purified by preparative HPLC as described in the general procedure to give the title compound (150 mg, 47%). ESI/MS (m/z): 589 (M+H)

### Preparation 102

### 4-(6-((((9H-Fluoren-9-yl)methoxy)carbonyl)amino)hexanamido)-1-naphthoic acid 7862B-4S Intermediate 1

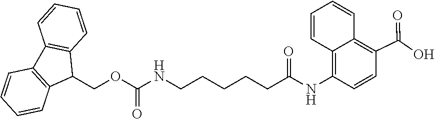 [SMILES: O=C(CCCCCNC(=O)OCC1c2ccccc2-c2ccccc21)Nc1ccc(C(=O)O)c2ccccc12]

6-((((9H-Fluoren-9-yl)methoxy)carbonyl)amino)hexanoic acid was coupled with methyl 4-aminonaphthalene-1-carboxylate with similar procedures as described for 4-(5-((tert-butoxycarbonyl)amino)pentanamido)-1-naphthoic acid. The residue was treated with 0.2N LiOH in MeOH and THF and then protected with Fmoc by using Fmoc-OSu and TEA to give the title compound. ESI/MS (m/z): 523 (M+H)

### Preparation 103

### (S)-7-((6-((((9H-Fluoren-9-yl)methoxy)carbonyl)amino)-1-(tert-butoxy)-1-oxohexan-2-yl)amino)-7-oxoheptanoic acid

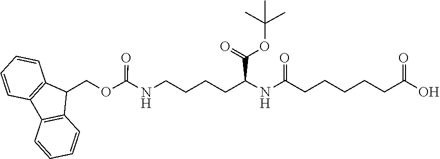 [SMILES: CC(C)(C)OC(=O)[C@H](CCCCNC(=O)OCC1c2ccccc2-c2ccccc21)NC(=O)CCCCCC(=O)O]

tert-Butyl N⁶-(((9H-fluoren-9-yl)methoxy)carbonyl)-L-lysinate was reacted with 7-(4-nitrophenoxy)-7-oxoheptanoic acid with TEA in DMF to afford the title product. ESI/MS (m/z): 567 (M+H).

### Preparation 104

### 1-(tert-Butyl) 2-(1,3-dioxoisoindolin-2-yl) (S)-4,4-difluoropyrrolidine-1,2-dicarboxylate 7824H-4FF Compound 1

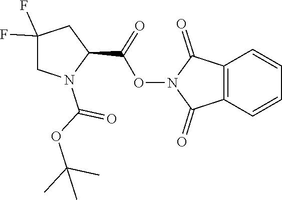 [SMILES: CC(C)(C)OC(=O)N1CC(F)(F)C[C@H]1C(=O)ON1C(=O)c2ccccc2C1=O]

A stirred solution of (S)-1-(tert-butoxycarbonyl)-4,4-difluoropyrrolidine-2-carboxylic acid (1.0 g, 4 mmol), N-hydroxyphthalimide (650 mg, 4 mmol) and DMAP (50 mg, 0.4 mmol) in anhydrous DCM (20 mL) was treated with N,N′-diisopropylcarbodiimide (0.69 mL) at rt. The mixture was allowed to stir until all the acid was consumed. The resulting mixture was concentrated under reduced pressure and purified by silica gel chromatography eluted with 0%-100% EtOAc in hexane to afford the title compound (1.35 g, 85%) as a white powder. ESI/MS (m/z): 299 (M-Boc+H).

### Preparation 105

### tert-Butyl 4,4-difluoro-2-((3aS)-3a,5,5-trimethylhexahydro-4,6-methanobenzo[d][1,3,2]dioxaborol-2-yl)pyrrolidine-1-carboxylate

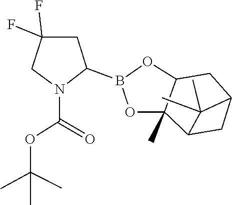

A screw-capped tube was charged with NiCl₂·6H₂O (67 mg, 0.28 mmol) and 4,4′-di-tert-butyl-2,2′-bipyridine (97 mg, 0.36 mmol) and evacuated and backfilled with Ar three times. THF (10 mL) was added and the resulting mixture stirred at rt overnight to afford a pale green suspension (A). A solution of B₂pin₂ (840 mg, 3.3 mmol) in THF (4 mL) was treated with MeLi (1.9 mL, 1.6M in Et₂O, 3 mmol) at 0° C. under Ar. The reaction mixture was warmed to rt and stirred for 1 h to afford a clear solution (B). A two-neck flask was charged with 1-(tert-butyl) 2-(1,3-dioxoisoindolin-2-yl) (S)-4,4-difluoropyrrolidine-1,2-dicarboxylate (1 mmol, 396 mg) and MgBr₂·OEt₂ (385 mg, 1.5 mmol) under Ar. The reaction flask was evacuated and backfilled with Ar three times. Suspension A (4 mL) was added via syringe. The mixture was stirred vigorously at rt until no granular MgBr₂·OEt₂ was observed (10 min). This suspension was cooled to 0° C. and treated with Solution B was added in one portion. After stirring for 1 h at 0° C., the reaction was warmed to rt and stirred for 1 h. Ice-water (10 mL) was added, the reaction mixture was concentrated, and the pH adjusted to 2 with 1N HCl. The reaction was extracted with EtOAc (3×20 mL). The organic phases were combined, washed with saturated aqueous NaCl, dried over Na₂SO₄, filtered, concentrated under reduced pressure to give a residue which was dissolved in anhydrous Et₂O (20 mL), and treated with (+)-pinanediol (340 mg). The reaction was stirred at rt overnight, filtered, concentrated under reduced pressure, and the residue purified by semi-preparative HPLC purification. Both the L/D isomers were collected and lyophilized to afford the title compound (0.25 g, 50%) as a pale-yellow solid. ESI/MS (m/z): 286 (M-Boc+H)

The following compound in Table 4 was synthesized in essentially the same manner as described for tert-butyl 4,4-difluoro-2-((3aS)-3a,5,5-trimethylhexahydro-4,6-methanobenzo[d][1,3,2]dioxaborol-2-yl)pyrrolidine-1-carboxylate.

TABLE 4

| | | | ESI/ |
|---|---|---|---|
| Prep | | | MS |
| # | Chemical Name | Structure | m/z |
| 106 | tert-Butyl (4S)-4- fluoro-2-((3aS)- 3a,5,5- trimethylhexahydro- 4,6- methanobenzo[d] [1,3,2]dioxaborol- 2-yl)pyrrolidine- 1-carboxylate | 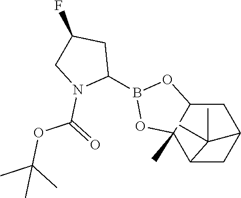 [SMILES: CC(C)(C)OC(=O)N1C[C@@H](F)CC1B1OC2CC3CC(C3(C)C)[C@]2(C)O1] | 268 (M − Boc + H) |

### Preparation 107

### 4,4-Difluoro-2-((3aS)-3a,5,5-trimethylhexahydro-4,6-methanobenzo[d][1,3,2]dioxaborol-2-yl)pyrrolidine HCl

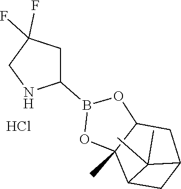

tert-Butyl 4,4-difluoro-2-((3aS)-3a,5,5-trimethylhexahydro-4,6-methanobenzo[d][1,3,2]dioxaborol-2-yl)pyrrolidine-1-carboxylate (0.25 g, 0.5 mmol) was dissolved in 5 mL of 4M HCl in 1,4-dioxane and allowed to stir at rt for 5 h. The resulting mixture was concentrated under reduced pressure to afford the title compound (0.2 g, 100%) as a pale-yellow solid.

The following compound in Table 5 was synthesized in essentially the same manner as described for 4,4-difluoro-2-((3aS)-3a,5,5-trimethylhexahydro-4,6-methanobenzo[d][1,3,2]dioxaborol-2-yl)pyrrolidine HCl.

TABLE 5

| | | | ESI/ |
|---|---|---|---|
| Prep | | | MS |
| # | Chemical Name | Structure | m/z |
| 108 | (4S)-4-Fluoro-2- ((3aS,4S,6S)- 3a,5,5- trimethylhexahydro- 4,6- methanobenzo[d] [1,3,2]dioxaborol- 2-yl)pyrrolidine HCl | 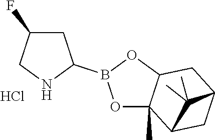 [SMILES: Cl ; CC1(C)[C@@H]2CC3OB(C4C[C@H](F)CN4)O[C@@]3(C)[C@H]1C2] | 268 (M + H) |

### Preparation 109

### 2-Amino-1-((2R,4S)-4-hydroxy-2-((3aS,4S,6S)-3a,5,5-trimethylhexahydro-4,6-methanobenzo[d][1,3,2]dioxaborol-2-yl)pyrrolidin-1-yl)ethan-1-one

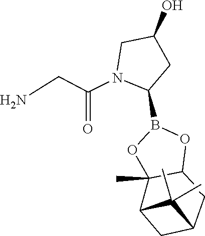

A stirred solution of (tert-butoxycarbonyl)glycine (0.35 g, 2 mmol) in anhydrous DMF (10 mL) was treated with HATU (0.8 g, 2.10 mmol) and (3S,5R)-5-((3aS,4S,6S)-3a,5,5-trimethylhexahydro-4,6-methanobenzo[d][1,3,2]dioxaborol-2-yl)pyrrolidin-3-ol HCl (0.77 g, 2.1 mmol) under ice-water bath cooling. DIPEA (0.76 mL, 4.6 mmol) was added dropwise. The resulting mixture was stirred at rt for 1 h and then concentrated under reduced pressure. The residue was dissolved in EtOAc (100 mL) and washed sequentially with 0.1N KHSO₄ (3×20 mL), aqueous NaHCO₃ (3×20 mL), and saturated aqueous NaCl (3×20 mL). The organic phase was dried over anhydrous MgSO₄, filtered, and concentrated under reduced pressure. The residue was purified by silica gel chromatography eluted with EtOAc. The residue was added to a solution of 4N HCl in dioxane (20 mL) under ice-water cooling. The reaction was stirred at rt for 2 h and then concentrated in under reduced pressure. The residue was co-evaporated with DCM (3×20 mL) under reduced pressure to afford the title compound (0.39 g, 55%) as a white powder.

The following compounds in Table 6 were synthesized in essentially the same manner as described for 2-amino-1-((2R,4S)-4-hydroxy-2-((3aS,4S,6S)-3a,5,5-trimethylhexahydro-4,6-methanobenzo[d][1,3,2]dioxaborol-2-yl)pyrrolidin-1-yl)ethan-1-one.

TABLE 6

| | | | ESI/ |
|---|---|---|---|
| Prep | | | MS |
| # | Chemical Name | Structure | m/z |
| 110 | (2R)-2-Amino-1-(4,4- difluoro-2-((3aS,4S,6S)- 3a,5,5- trimethylhexahydro- 4,6- methanobenzo[d][1,3,2] dioxaborol-2-yl) pyrrolidin-1- yl)propan-1-one HCl | 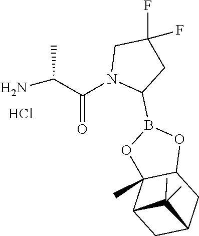 [SMILES: Cl ; C[C@@H](N)C(=O)N1CC(F)(F)CC1B1OC2C[C@@H]3C[C@@H](C3(C)C)[C@]2(C)O1] | 357 (M + H) |
| 111 | (2R)-2-Amino-1-((4S)-4- fluoro-2-((3aS,4S,6S)- 3a,5,5-trimethylhexahydro- 4,6- methanobenzo[d][1,3,2] dioxaborol-2-yl) pyrrolidin-1- yl)propan-1-one HCl | 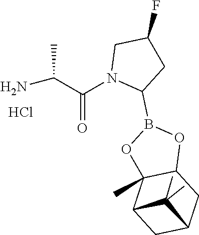 [SMILES: Cl ; C[C@@H](N)C(=O)N1C[C@@H](F)CC1B1OC2C[C@@H]3C[C@@H](C3(C)C)[C@]2(C)O1] | 339 (M + H) |
| 112 | N-(2-((2R,4S)-4-Hydroxy- 2-((3aS,4S,6S)-3a,5,5- trimethylhexahydro-4,6- methanobenzo[d][1,3,2] dioxaborol-2-yl) pyrrolidin-1-yl)- 2-oxoethyl)-4- ((methylamino)methyl) benzamide HCl | 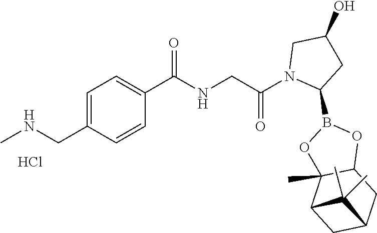 | |
| 113 | (1r,4R)-4-(Aminomethyl)- N-((2R)-1-oxo-1-((2R)-2- ((3aS,4S,6S)-3a,5,5- trimethylhexahydro-4,6- methanobenzo[d][1,3,2] dioxaborol-2-yl)pyrrolidin-1- yl)propan-2-yl)cyclohexane- 1-carboxamide | 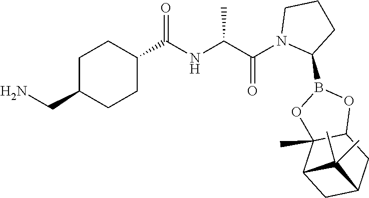 | 460 (M + H) |
| 114 | 4-(5-Aminopentanamido)- N-((2R)-1-oxo-1-((2R)-2- ((3aS,4S,6S)-3a,5,5- trimethylhexahydro-4,6- methanobenzo[d][1,3,2] dioxaborol-2-yl)pyrrolidin-1- yl)propan-2-yl)-1- naphthamide | 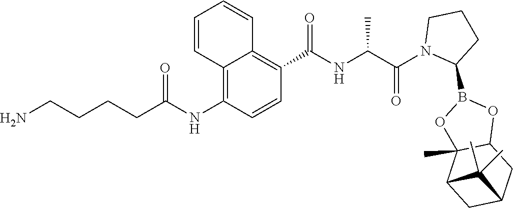 | 589 (M + H) |
| 115 | (9H-Fluoren-9-yl)methyl (S,E)-(1-(4-(2- aminoethyl)piperazin-1-yl)- 5- (((dimethylamino)((2,2,4,6,7- pentamethyl-2,3- dihydrobenzofuran)-5- sulfonamido)methylene) amino)-1-oxopentan-2- yl)carbamate HCl | 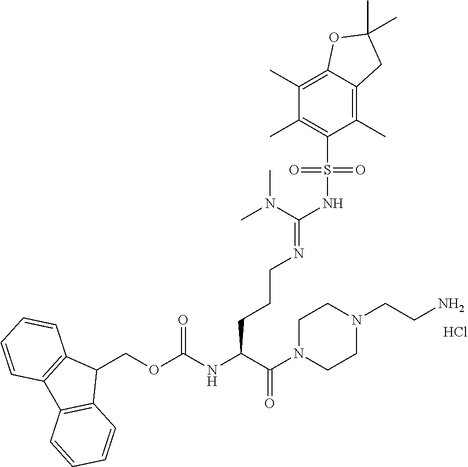 [SMILES: Cc1c(C)c(S(=O)(=O)N/C(=N/CCC[C@H](NC(=O)OCC2c3ccccc3-c3ccccc32)C(=O)N2CCN(CCN)CC2)N(C)C)c(C)c2c1OC(C)(C)C2 ; Cl] | 788 (M + H) |

### Preparation 116

### (9R,19S)-19-(3-((E)-2,3-Bis(tert-butoxycarbonyl)guanidino)propyl)-9-(tert-butoxycarbonyl)-1-(9H-fluoren-9-yl)-3,11,17,20-tetraoxo-2-oxa-4,10,18,21-tetraazahexacosan-26-oic acid

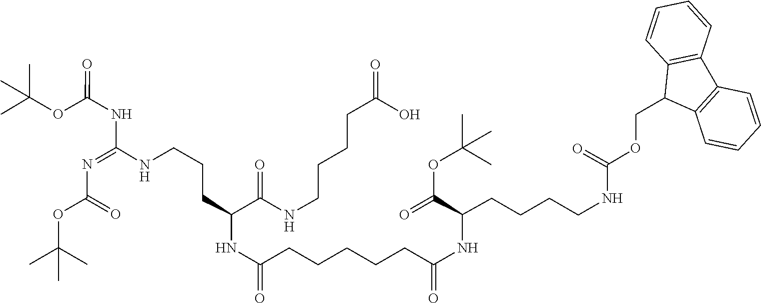 [SMILES: CC(C)(C)OC(=O)/N=C(\NCCC[C@H](NC(=O)CCCCCC(=O)N[C@H](CCCCNC(=O)OCC1c2ccccc2-c2ccccc21)C(=O)OC(C)(C)C)C(=O)NCCCCC(=O)O)NC(=O)OC(C)(C)C]

The initial resin loading with 5-((((9H-fluoren-9-yl)methoxy)carbonyl)amino)pentanoic acid was performed as described in SP-1. The Fmoc protecting group was removed according to SP-3 and then coupled to (E)-N²-(((9H-fluoren-9-yl)methoxy)carbonyl)-N^{w},N^{w′}-bis(tert-butoxycarbonyl)-L-arginine according to SP-2. The Fmoc protecting group was removed according to SP-3 and coupled with 7-(4-nitrophenoxyl)-7-oxoheptanoic acid according to SP-2 without the addition of HATU, then was coupled with tert-butyl N⁶-(((9H-fluoren-9-yl)methoxy)carbonyl)-L-lysinate according to SP-2 except using PyBrop instead of HATU. The peptide was cleaved from the resin according to SP-4 to give the title compound (45 mg, 44%). ESI/MS (m/z): 1023 (M+H).

### Preparation 117

### 4-((5-((S)-2-Amino-N,N′-di-Boc-5-guanidinopentanamido)pentanamido)methyl)-N-((2R)-1-oxo-1-((2R)-2-((3aS,4S,6S)-3a,5,5-trimethylhexahydro-4,6-methanobenzo[d][1,3,2]dioxaborol-2-yl)pyrrolidin-1-yl)propan-2-yl)benzamide

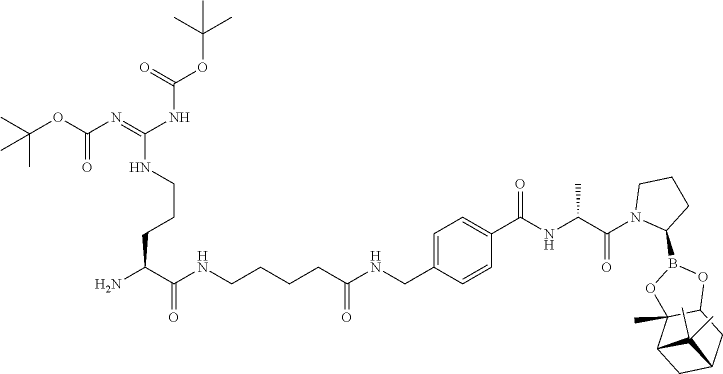

4-(Aminomethyl)-N-((2R)-1-oxo-1-((2R)-2-((3aS,4S,6S)-3a,5,5-trimethylhexahydro-4,6-methanobenzo[d][1,3,2]dioxaborol-2-yl)pyrrolidin-1-yl)propan-2-yl)benzamide HCl was coupled to N-Boc-5-aminovaleric acid with HATU and DIPEA in DMF according to SP-5. The Boc protecting group was removed with 4N HCl in 1,4-dioxane. The resulting amine was coupled to (S,E)-11-((((9H-fluoren-9-yl)methoxy)carbonyl)amino)-6-((tert-butoxycarbonyl)amino)-2,2-dimethyl-4,12-dioxo-3-oxa-5,7,13-triazaoctadec-5-en-18-oic acid with HATU and DIPEA in DMF according to the SP-5. The Fmoc protecting group was removed with piperidine (5%) in DMF to give the title compound (250 mg, 55%). ES/MS (m/z): 909 (M+H).

### Preparation 118

### tert-Butyl (3S)-3-(2-((S)-2-amino-5-(3-((2,2,4,6,7-pentamethyl-2,3-dihydrobenzofuran-5-yl)sulfonyl)guanidino)pentanamido)acetamido)-4-oxo-4-((4-(((2R)-1-oxo-1-((2R)-2-((3aS,4S,6S)-3a,5,5-trimethylhexahydro-4,6-methanobenzo[d][1,3,2]dioxaborol-2-yl)pyrrolidin-1-yl)propan-2-yl)carbamoyl)benzyl)amino)butanoate

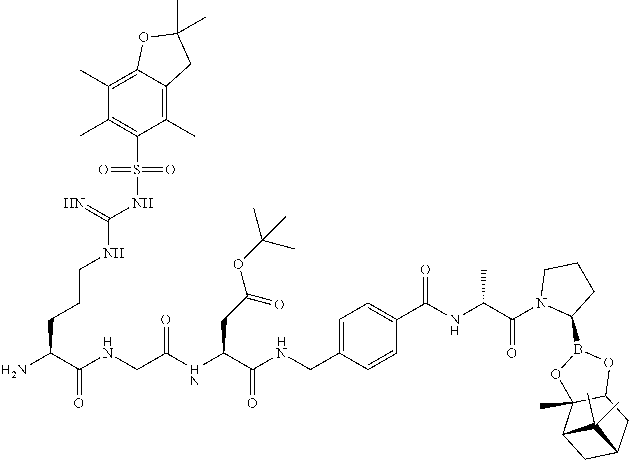

(5S,11S)-11-(2-(tert-Butoxy)-2-oxoethyl)-1-(9H-fluoren-9-yl)-3,6,9-trioxo-5-(3-(3-((2,2,4,6,7-pentamethyl-2,3-dihydrobenzofuran-5-yl)sulfonyl)guanidino)propyl)-2-oxa-4,7,10-triazadodecan-12-oic acid was coupled to 4-(aminomethyl)-N-((2R)-1-oxo-1-((2R)-2-((3aS,4S,6S)-3a,5,5-trimethylhexahydro-4,6-methanobenzo[d][1,3,2]dioxaborol-2-yl)pyrrolidin-1-yl)propan-2-yl)benzamide HCl with HATU and DIPEA in DMF according to the SP-5. The Fmoc protecting group was then removed with piperidine (5%) in DMF to give the title compound (95 mg, 87%). ESI/MS (m/z): 1091 (M+H).

The following compound in Table 7 was synthesized in essentially the same manner as described for tert-butyl (3S)-3-(2-((S)-2-amino-5-(3-((2,2,4,6,7-pentamethyl-2,3-dihydrobenzofuran-5-yl)sulfonyl)guanidino)pentanamido)acetamido)-4-oxo-4-((4-(((2R)-1-oxo-1-((2R)-2-((3aS,4S,6S)-3a,5,5-trimethylhexahydro-4,6-methanobenzo[d][1,3,2]dioxaborol-2-yl)pyrrolidin-1-yl)propan-2-yl)carbamoyl)benzyl)amino)butanoate.

TABLE 7

| Prep | | ESI/MS |
|---|---|---|
| # | Structure | m/z |
| 119 | 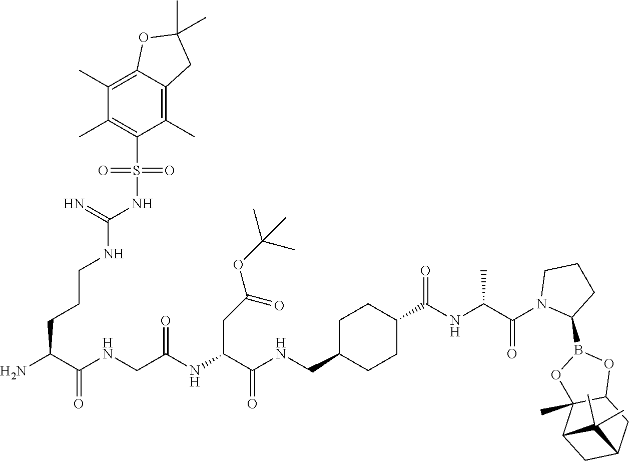 | 1097 (M + H) |
| | tert-Butyl (3R)-3-(2-((S)-2-amino-5-(3-((2,2,4,6,7-pentamethyl-2,3- | |
| | dihydrobenzofuran-5-yl)sulfonyl)guanidino)pentanamido)acetamido)-4-oxo- | |
| | 4-((((1R,4R)-4-(((2R)-1-oxo-1-((2R)-2-((3aS,4S,6S)-3a,5,5- | |
| | trimethylhexahydro-4,6-methanobenzo[d][1,3,2]dioxaborol-2-yl)pyrrolidin- | |
| | 1-yl)propan-2-yl)carbamoyl)cyclohexyl)methyl)amino)butanoate | |

Preparation 120

### tert-Butyl (3S)-3-(2-((S)-2-amino-6-(3-((2,2,4,6,7-pentamethyl-2,3-dihydrobenzofuran-5-yl)sulfonyl)guanidino)hexanamido)acetamido)-4-oxo-4-((4-(((2R)-1-oxo-1-((2R)-2-((3aS,4S,6S)-3a,5,5-trimethylhexahydro-4,6-methanobenzo[d][1,3,2]dioxaborol-2-yl)pyrrolidin-1-yl)propan-2-yl)carbamoyl)benzyl)amino)butanoate

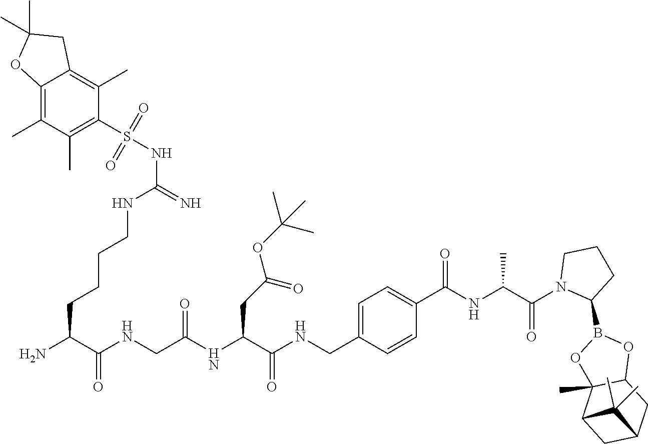

The initial resin loading with 4-(((((9H-fluoren-9-yl)methoxy)carbonyl)amino)methyl)benzoic acid was performed as described in SP-1. The Fmoc protecting group was removed using pyrrolidine (5%) in DMF and then was coupled (S)-2-((((9H-fluoren-9-yl)methoxy)carbonyl)amino)-4-(tert-butoxy)-4-oxobutanoic acid according to SP-2. The Fmoc protecting group was removed using pyrrolidine (5%) in DMF and then was coupled with (((9H-fluoren-9-yl)methoxy)carbonyl)glycine according to SP-2. The Fmoc protecting group was removed using pyrrolidine (5%) in DMF and then was coupled with N²-(((9H-fluoren-9-yl)methoxy)carbonyl)-N⁶—(N-((2,2,4,6,7-pentamethyl-2,3-dihydrobenzofuran-5-yl)sulfonyl)carbamimidoyl)-L-lysine according to SP-2. The peptide was cleaved from the resin according to SP-4 and was coupled with (2R)-2-amino-1-((2R)-2-((3aS,4S,6S)-3a,5,5-trimethylhexahydro-4,6-methanobenzo[d][1,3,2]dioxaborol-2-yl)pyrrolidin-1-yl)propan-1-one HCl according to the SP-5 except the addition of HOAt. The Fmoc protecting group was removed with pyrrolidine (5%) in DMF to give the title compound (320 mg, 29%). ESI/MS (m/z): 1105 (M+H).

The following compounds in Table 8 were synthesized in essentially the same manner as described for tert-butyl (3S)-3-(2-((S)-2-amino-6-(3-((2,2,4,6,7-pentamethyl-2,3-dihydrobenzofuran-5-yl)sulfonyl)guanidino)hexanamido)acetamido)-4-oxo-4-((4-(((2R)-1-oxo-1-((2R)-2-((3aS,4S,6S)-3a,5,5-trimethylhexahydro-4,6-methanobenzo[d][1,3,2]dioxaborol-2-yl)pyrrolidin-1-yl)propan-2-yl)carbamoyl)benzyl)amino)butanoate.

TABLE 8

| Prep | | ESI/MS |
|---|---|---|
| # | Structure | m/z |
| 121 | 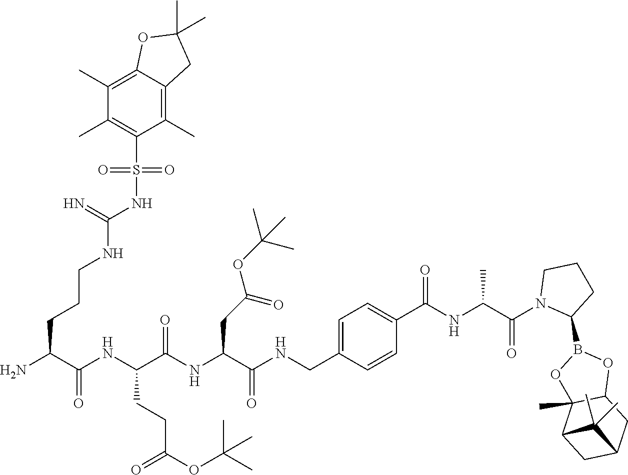 | 1219 (M + H) |
| | tert-Butyl (4S)-4-((S)-2-amino-5-(3-((2,2,4,6,7-pentamethyl-2,3- | |
| | dihydrobenzofuran-5-yl)sulfonyl)guanidino)pentanamido)-5-(((2S)-4-(tert- | |
| | butoxy)-1,4-dioxo-1-((4-(((2R)-1-oxo-1-((2R)-2-((3aS,4S,6S)-3a,5,5- | |
| | trimethylhexahydro-4,6-methanobenzo[d][1,3,2]dioxaborol-2-yl)pyrrolidin- | |
| | 1-yl)propan-2-yl)carbamoyl)benzyl)amino)butan-2-yl)amino)-5- | |
| | oxopentanoate | |
| 122 | 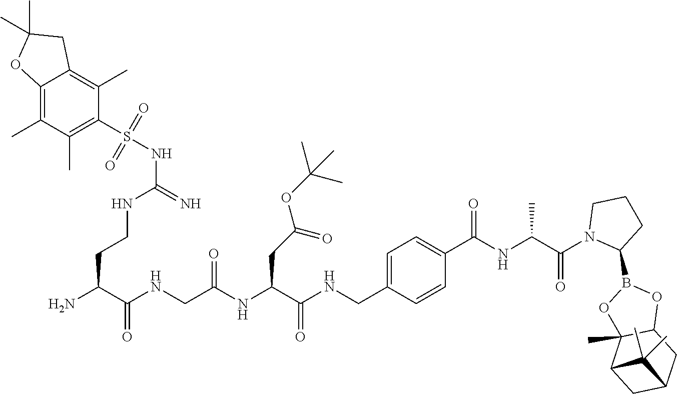 | 1077 (M + H) |
| | tert-Butyl (3S)-3-(2-((S)-2-amino-4-(3-((2,2,4,6,7-pentamethyl-2,3- | |
| | dihydrobenzofuran-5-yl)sulfonyl)guanidino)butanamido)acetamido)-4-oxo- | |
| | 4-((4-(((2R)-1-oxo-1-((2R)-2-((3aS,4S,6S)-3a,5,5-trimethylhexahydro-4,6- | |
| | methanobenzo[d][1,3,2]dioxaborol-2-yl)pyrrolidin-1-yl)propan-2- | |
| | yl)carbamoyl)benzyl)amino)butanoate | |
| 123 | 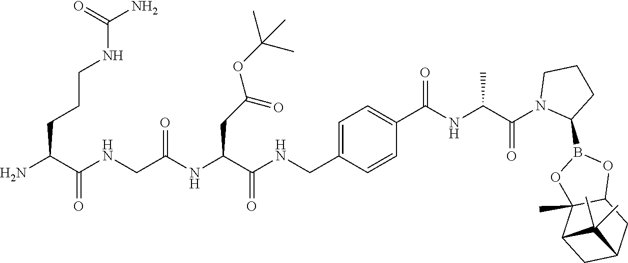 | 839 (M + H) |
| | tert-Butyl (3S)-3-(2-((S)-2-amino-5-ureidopentanamido)acetamido)-4-oxo-4- | |
| | ((4-(((2R)-1-oxo-1-((2R)-2-((3aS,4S,6S)-3a,5,5-trimethylhexahydro-4,6- | |
| | methanobenzo[d][1,3,2]dioxaborol-2-yl)pyrrolidin-1-yl)propan-2- | |
| | yl)carbamoyl)benzyl)amino)butanoate | |

### Preparation 124

### 4-((5-((S)-2-Amino-5-(3-((2,2,4,6,7-pentamethyl-2,3-dihydrobenzofuran-5-yl)sulfonyl)guanidino)pentanamido)pentanamido)methyl)-N-((2R)-1-((2R,4R)-4-hydroxy-2-((3aS,4S,6S)-3a,5,5-trimethylhexahydro-4,6-methanobenzo[d][1,3,2]dioxaborol-2-yl)pyrrolidin-1-yl)-1-oxopropan-2-yl)benzamide

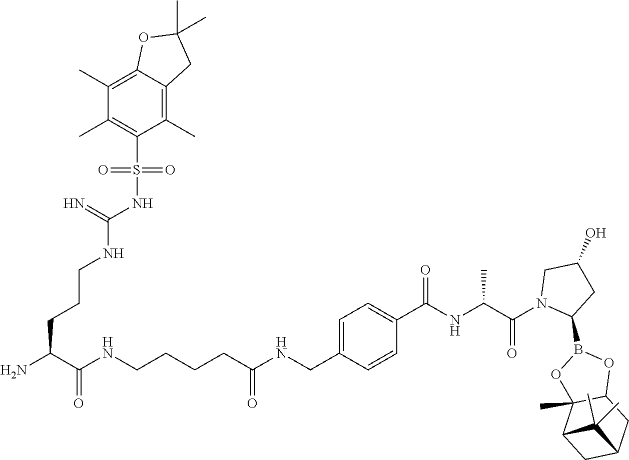

(tert-Butoxycarbonyl)-D-alanine was coupled with (3R,5R)-5-((3aS,4S,6S)-3a,5,5-trimethylhexahydro-4,6-methanobenzo[d][1,3,2]dioxaborol-2-yl)pyrrolidin-3-ol hydrochloride according to SP-5. The Boc protecting group was removed using 4N HCl in 1,4-dioxane, which was then coupled with (S)-4-(1-(9H-fluoren-9-yl)-3,6,12-trioxo-5-(3-(3-((2,2,4,6,7-pentamethyl-2,3-dihydrobenzofuran-5-yl)sulfonyl)guanidino)propyl)-2-oxa-4,7,13-triazatetradecan-14-yl)benzoic acid according to SP-5 using HATU and DIPEA in DMF. The Fmoc protecting group was removed according to SP-3 to give title compound (105 mg, 54%). ESI/MS (m/z): 978 (M+H).

The following compound in Table 9 was synthesized in essentially the same manner as described for 4-((5-((S)-2-amino-5-(3-((2,2-dimethyl-2,3-dihydrobenzofuran-5-yl)sulfonyl)guanidino)pentanamido)pentanamido)methyl)-N-((2R)-1-oxo-1-((2R)-2-((3aS,4S,6S)-3a,5,5-trimethylhexahydro-4,6-methanobenzo[d][1,3,2]dioxaborol-2-yl)pyrrolidin-1-yl)propan-2-yl)benzamide.

TABLE 9

| | | ESI/MS |
|---|---|---|
| Prep # | Structure | m/z |
| 125 | 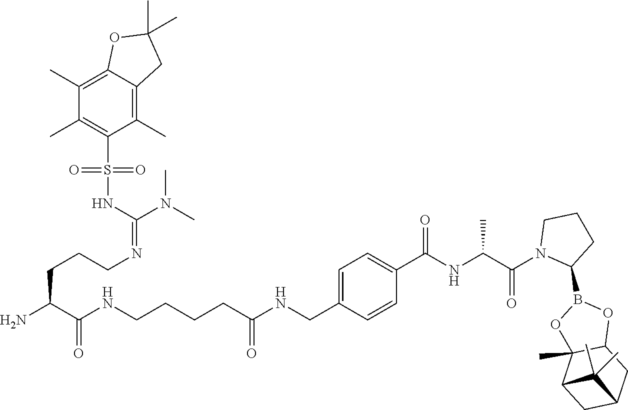 | 990 (M + H) |
| | 4-((S,Z)-8-Amino-2-methyl-9,15-dioxo-3-((2,2,4,6,7-pentamethyl-2,3- | |
| | dihydrobenzofuran)-5-sulfonamido)-2,4, 10,16-tetraazaheptadec-3-en-17-yl)- | |
| | N-((2R)-1-oxo-1-((2R)-2-((3aS,4S,6S)-3a,5,5-trimethylhexahydro-4,6- | |
| | methanobenzo[d][1,3,2]dioxaborol-2-yl)pyrrolidin-1-yl)propan-2- | |
| | yl)benzamide | |

### Preparation 126

### (S)-4-((5-(5-(3-((2,2,4,6,7-Pentamethyl-2,3-dihydrobenzofuran-5-yl)sulfonyl)guanidino)-2-(2-(4,7,10-tris(2-(tert-butoxy)-2-oxoethyl)-1,4,7,10-tetraazacyclododecan-1-yl)acetamido)pentanamido)pentyl)carbamoyl)-1-naphthoic acid

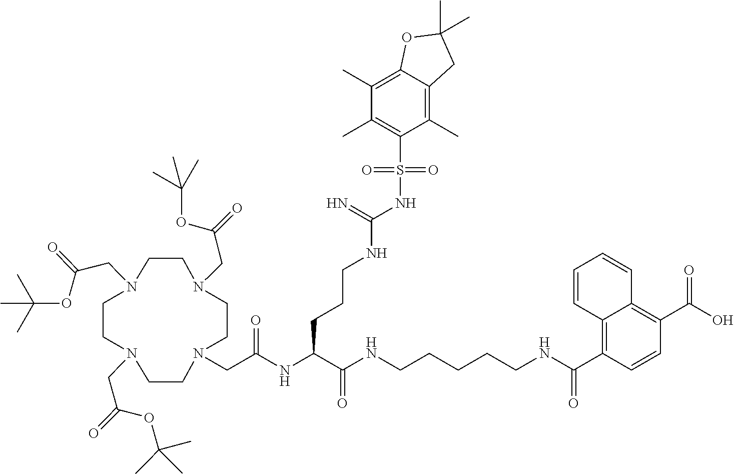 [SMILES: Cc1c(C)c(S(=O)(=O)NC(=N)NCCC[C@H](NC(=O)CN2CCN(CC(=O)OC(C)(C)C)CCN(CC(=O)OC(C)(C)C)CCN(CC(=O)OC(C)(C)C)CC2)C(=O)NCCCCCNC(=O)c2ccc(C(=O)O)c3ccccc23)c(C)c2c1OC(C)(C)C2]

The initial resin loading with naphthalene-1,4-dicarboxylic acid was performed according to SP-1 and then coupled with (9H-fluoren-9-yl)methyl (5-aminopentyl)carbamate hydrochloride according to SP-2 with PyBroP instead of HATU as the coupling reagents. The Fmoc protecting group was removed according to SP-3 and coupled with N²-(((9H-fluoren-9-yl)methoxy)carbonyl)-N^{w}-((2,2,4,6,7-pentamethyl-2,3-dihydrobenzofuran-5-yl)sulfonyl)-L-arginine according to SP-2 using PyBroP as the coupling reagent. The Fmoc group was removed according to SP-3 and coupled with 2-(4,7,10-tris(2-(tert-butoxy)-2-oxoethyl)-1,4,7,10-tetraazacyclododecan-1-yl)acetic acid according to SP-2 with PyBroP instead of HATU as the coupling reagent. The compound was cleaved from the resin according to SP-4 to give the title compound (0.04 g, 27%). ESI/MS (m/z): 1265 (M+H).

The following compound in Table 10 was synthesized in essentially the same manner as described for (S)-4-((5-(2-(2-(7-(2-Amino-2-oxoethyl)-4,10-bis(2-(tert-butoxy)-2-oxoethyl)-1,4,7,10-tetraazacyclododecan-1-yl)acetamido)-5-(3-((2,2,4,5,7-pentamethyl-2,3-dihydrobenzofuran-6-yl)sulfonyl)guanidino)pentanamido)-N-methylpentanamido)methyl)benzoic acid.

TABLE 10

| | | ESI/MS |
|---|---|---|
| Prep # | Structure | m/z |
| 127 | 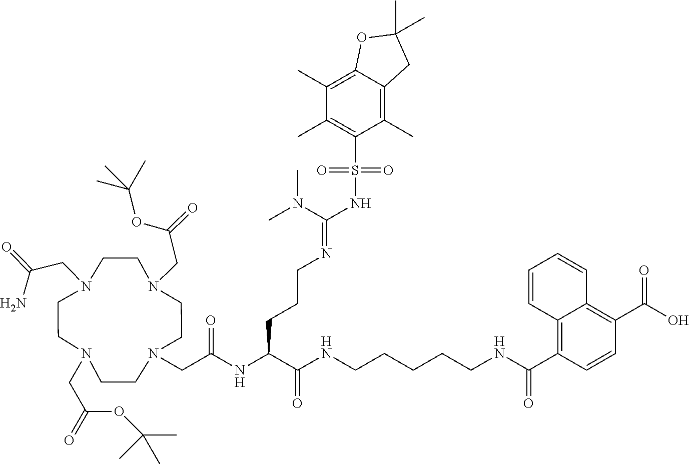 [SMILES: Cc1c(C)c(S(=O)(=O)N/C(=N/CCC[C@H](NC(=O)CN2CCN(CC(=O)OC(C)(C)C)CCN(CC(N)=O)CCN(CC(=O)OC(C)(C)C)CC2)C(=O)NCCCCCNC(=O)c2ccc(C(=O)O)c3ccccc23)N(C)C)c(C)c2c1OC(C)(C)C2] | |
| | (S,E)-4-((5-(2-(2-(7-(2-Amino-2-oxoethyl)-4,10-bis(2-(tert-butoxy)-2- | |
| | oxoethyl)-1,4,7,10-tetraazacyclododecan-1-yl)acetamido)-5- | |
| | (((dimethylamino)((2,2,4,6,7-pentamethyl-2,3-dihydrobenzofuran)-5- | |
| | sulfonamido)methylene)amino)pentanamido)pentyl)carbamoyl)-1-naphthoic | |
| | acid | |

The following compound in Table 11 was synthesized in essentially the same manner as described for (S)-4-((5-(2-(2-(7-(2-amino-2-oxoethyl)-4,10-bis(2-(tert-butoxy)-2-oxoethyl)-1,4,7,10-tetraazacyclododecan-1-yl)acetamido)-5-(3-((2,2,4,5,7-pentamethyl-2,3-dihydrobenzofuran-6-yl)sulfonyl)guanidino)pentanamido)pentyl)carbamoyl)-1-naphthoic acid.

TABLE 11

| | | ESI/MS |
|---|---|---|
| Prep # | Structure | m/z |
| 128 | 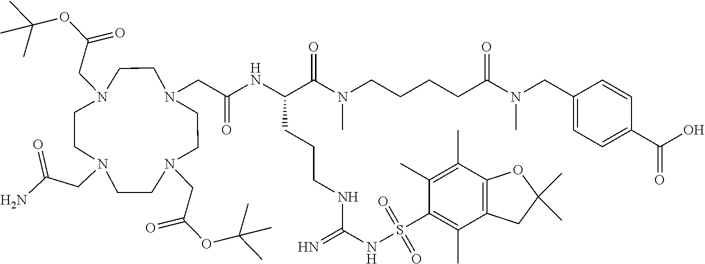 [SMILES: Cc1c(C)c(S(=O)(=O)NC(=N)NCCC[C@H](NC(=O)CN2CCN(CC(=O)OC(C)(C)C)CCN(CC(N)=O)CCN(CC(=O)OC(C)(C)C)CC2)C(=O)N(C)CCCCC(=O)N(C)Cc2ccc(C(=O)O)cc2)c(C)c2c1OC(C)(C)C2] | 1185 (M + H) |
| | (S)-4-((5-(2-(2-(7-(2-Amino-2-oxoethyl)-4,10-bis(2-(tert-butoxy)-2- | |
| | oxoethyl)-1,4,7,10-tetraazacyclododecan-1-yl)acetamido)-N-methyl-5-(3- | |
| | ((2,2,4,6,7-pentamethyl-2,3-dihydrobenzofuran-5- | |
| | yl)sulfonyl)guanidino)pentanamido)-N-methylpentanamido)methyl)benzoic | |
| | acid | |

### Preparation 129

### 2,2′,2″-(10-(2-(((S)-1-((5-((4-((2-((2R,4S)-2-Borono-4-hydroxypyrrolidin-1-yl)-2-oxoethyl)carbamoyl)benzyl)(methyl)amino)-5-oxopentyl)amino)-5-guanidino-1-oxopentan-2-yl)amino)-2-oxoethyl)-1,4,7,10-tetraazacyclododecane-1,4,7-triyl)triacetic acid

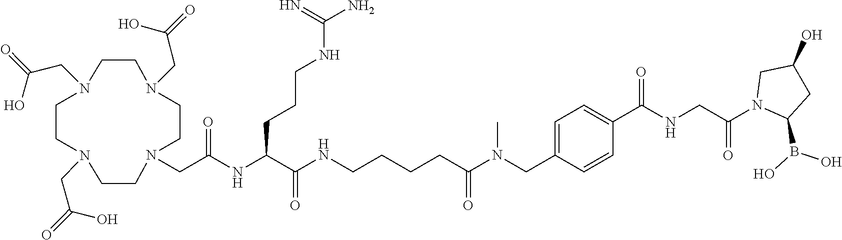 [SMILES: CN(Cc1ccc(C(=O)NCC(=O)N2C[C@@H](O)C[C@H]2B(O)O)cc1)C(=O)CCCCNC(=O)[C@H](CCCNC(=N)N)NC(=O)CN1CCN(CC(=O)O)CCN(CC(=O)O)CCN(CC(=O)O)CC1]

(S)-5-(5-(3-((2,2,4,5,7-Pentamethyl-2,3-dihydrobenzofuran-6-yl)sulfonyl)guanidino)-2-(2-(4,7,10-tris(2-(tert-butoxy)-2-oxoethyl)-1,4,7,10-tetraazacyclododecan-1-yl)acetamido)pentanamido)pentanoic acid was coupled with N-(2-((2R,4S)-4-hydroxy-2-((3aS,4S,6S)-3a,5,5-trimethylhexahydro-4,6-methanobenzo[d][1,3,2]dioxaborol-2-yl)pyrrolidin-1-yl)-2-oxoethyl)-4-((methylamino)methyl)benzamide hydrochloride according to SP-5 and the protecting groups (—Pbf, -tBu, and pinanediol) were removed according to SP-6 to afford the title compound as a white powder (15 mg, 25%). ESI/MS (m/z): 959 (M−H₂O+H).

The following compounds in Table 12 were synthesized in essentially the same manner as described for 2,2′,2″-(10-(2-(((S)-1-((5-((4-((2-((2R,4S)-2-borono-4-hydroxypyrrolidin-1-yl)-2-oxoethyl)carbamoyl)benzyl)(methyl)amino)-5-oxopentyl)amino)-5-guanidino-1-oxopentan-2-yl)amino)-2-oxoethyl)-1,4,7,10-tetraazacyclododecane-1,4,7-triyl)triacetic acid.

TABLE 12

| | | ESI/MS |
|---|---|---|
| Prep # | Structure | m/z |
| 130 | 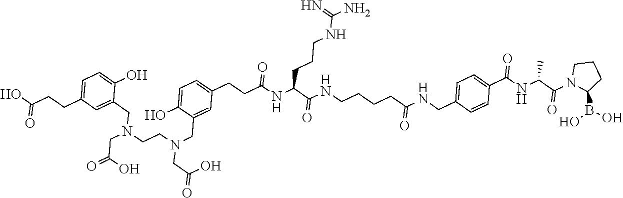 [SMILES: C[C@@H](NC(=O)c1ccc(CNC(=O)CCCCNC(=O)[C@H](CCCNC(=N)N)NC(=O)CCc2ccc(O)c(CN(CCN(CC(=O)O)Cc3cc(CCC(=O)O)ccc3O)CC(=O)O)c2)cc1)C(=O)N1CCC[C@H]1B(O)O] | 1071 (M − H₂O + H) |
| | 3-(3-(((2-((5-(3-(((S)-1-((5-((4-(((R)-1-((R)-2-Boronopyrrolidin-1-yl)-1- | |
| | oxopropan-2-yl)carbamoyl)benzyl)amino)-5-oxopentyl)amino)-5-guanidino- | |
| | 1-oxopentan-2-yl)amino)-3-oxopropyl)-2- | |
| | hydroxybenzyl)(carboxymethyl)amino)ethyl)(carboxymethyl)amino)methyl)- | |
| | 4-hydroxyphenyl)propanoic acid | |
| 131 | 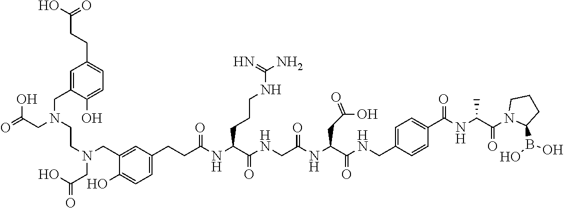 [SMILES: C[C@@H](NC(=O)c1ccc(CNC(=O)[C@H](CC(=O)O)NC(=O)CNC(=O)[C@H](CCCNC(=N)N)NC(=O)CCc2ccc(O)c(CN(CCN(CC(=O)O)Cc3cc(CCC(=O)O)ccc3O)CC(=O)O)c2)cc1)C(=O)N1CCC[C@H]1B(O)O] | 1145 (M − H₂O + H) |
| | (S)-4-((4-(((R)-1-((R)-2-Boronopyrrolidin-1-yl)-1-oxopropan-2- | |
| | yl)carbamoyl)benzyl)amino)-3-(2-((S)-2-(3-(3-(((2-((5-(2-carboxyethyl)-2- | |
| | hydroxybenzyl)(carboxymethyl)amino)ethyl)(carboxymethyl)amino)methyl)- | |
| | 4-hydroxyphenyl)propanamido)-5-guanidinopentanamido)acetamido)-4- | |
| | oxobutanoic acid | |

### Preparation 132

### 2,2′,2″-(10-((4S,10S)-1-(4-(((R)-1-((R)-2-Boronopyrrolidin-1-yl)-1-oxopropan-2-yl)carbamoyl)phenyl)-4-(carboxymethyl)-10-(3-guanidinopropyl)-3,6,9,12-tetraoxo-2,5,8,11-tetraazatridecan-13-yl)-1,4,7,10-tetraazacyclododecane-1,4,7-triyl)triacetic acid

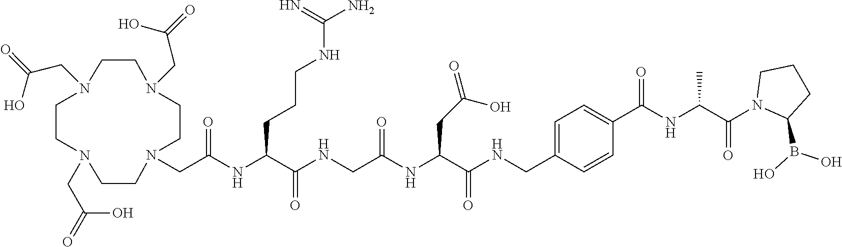 [SMILES: C[C@@H](NC(=O)c1ccc(CNC(=O)[C@H](CC(=O)O)NC(=O)CNC(=O)[C@H](CCCNC(=N)N)NC(=O)CN2CCN(CC(=O)O)CCN(CC(=O)O)CCN(CC(=O)O)CC2)cc1)C(=O)N1CCC[C@H]1B(O)O]

tert-Butyl (3S)-3-(2-((S)-2-amino-5-(3-((2,2,4,6,7-pentamethyl-2,3-dihydrobenzofuran-5-yl)sulfonyl)guanidino)pentanamido)acetamido)-4-oxo-4-((4-(((2R)-1-oxo-1-((2R)-2-((3aS,4S,6S)-3a,5,5-trimethylhexahydro-4,6-methanobenzo[d][1,3,2]dioxaborol-2-yl)pyrrolidin-1-yl)propan-2-yl)carbamoyl)benzyl)amino)butanoate was coupled with 2,2′,2″-[10-[2-[(2,5-dioxopyrrolidin-1-yl)oxy]-2-oxoethyl]-1,4,7,10-tetraazacyclododecane-1,4,7-triyl]triacetic acid in anhydrous DMF and TEA. The —Pbf, -OtBu, and pinanediol protecting groups were removed according to SP-6 to afford the title product (12.8 mg, 46%) as a white powder. ESI/MS (m/z): 1016 (M−H₂O+H).

The following compounds in Table 13 were synthesized in essentially the same manner as described for 2,2′,2″-(10-((4S,10S)-1-(4-(((R)-1-((R)-2-boronopyrrolidin-1-yl)-1-oxopropan-2-yl)carbamoyl)phenyl)-4-(carboxymethyl)-10-(3-guanidinopropyl)-3,6,9,12-tetraoxo-2,5,8,11-tetraazatridecan-13-yl)-1,4,7,10-tetraazacyclododecane-1,4,7-triyl)triacetic acid.

TABLE 13

| | | ESI/MS |
|---|---|---|
| Prep # | Structure | m/z |
| 133 | 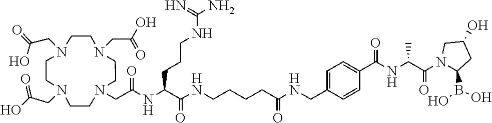 [SMILES: C[C@@H](NC(=O)c1ccc(CNC(=O)CCCCNC(=O)[C@H](CCCNC(=N)N)NC(=O)CN2CCN(CC(=O)O)CCN(CC(=O)O)CCN(CC(=O)O)CC2)cc1)C(=O)N1C[C@H](O)C[C@H]1B(O)O] | 959 (M − H₂O + H) |
| | 2,2′,2″-(10-(2-(((S)-1-((5-((4-(((R)-1-((2R,4R)-2-Borono-4-hydroxypyrrolidin- | |
| | 1-yl)-1-oxopropan-2-yl)carbamoyl)benzyl)amino)-5-oxopentyl)amino)-5- | |
| | guanidino-1-oxopentan-2-yl)amino)-2-oxoethyl)-1,4,7,10- | |
| | tetraazacyclododecane-1,4,7-triyl)triacetic acid | |
| 134 | 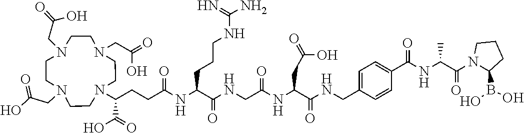 [SMILES: C[C@@H](NC(=O)c1ccc(CNC(=O)[C@H](CC(=O)O)NC(=O)CNC(=O)[C@H](CCCNC(=N)N)NC(=O)CC[C@H](C(=O)O)N2CCN(CC(=O)O)CCN(CC(=O)O)CCN(CC(=O)O)CC2)cc1)C(=O)N1CCC[C@H]1B(O)O] | 1089 (M − H₂O + H) |
| | 2,2′,2″-(10-((4S,10S,15R)-1-(4-(((R)-1-((R)-2-Boronopyrrolidin-1-yl)-1- | |
| | oxopropan-2-yl)carbamoyl)phenyl)-15-carboxy-4-(carboxymethyl)-10-(3- | |
| | guanidinopropyl)-3,6,9,12-tetraoxo-2,5,8,11-tetraazapentadecan-15-yl)- | |
| | 1,4,7,10-tetraazacyclododecane-1,4,7-triyl)triacetic acid | |
| 135 | 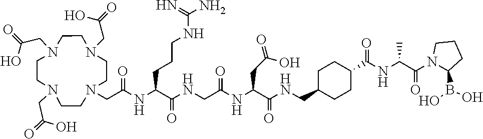 [SMILES: C[C@@H](NC(=O)[C@H]1CC[C@H](CNC(=O)[C@H](CC(=O)O)NC(=O)CNC(=O)[C@H](CCCNC(=N)N)NC(=O)CN2CCN(CC(=O)O)CCN(CC(=O)O)CCN(CC(=O)O)CC2)CC1)C(=O)N1CCC[C@H]1B(O)O] | 1022 (M − H₂O + H) |
| | 2,2′,2″-(10-((4S,10S)-1-((1R,4S)-4-(((R)-1-((R)-2-Boronopyrrolidin-1-yl)-1- | |
| | oxopropan-2-yl)carbamoyl)cyclohexyl)-4-(carboxymethyl)-10-(3- | |
| | guanidinopropyl)-3,6,9,12-tetraoxo-2,5,8,11-tetraazatridecan-13-yl)-1,4,7,10- | |
| | tetraazacyclododecane-1,4,7-triyl)triacetic acid | |
| 136 | 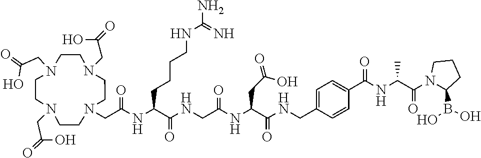 [SMILES: C[C@@H](NC(=O)c1ccc(CNC(=O)[C@H](CC(=O)O)NC(=O)CNC(=O)[C@H](CCCCNC(=N)N)NC(=O)CN2CCN(CC(=O)O)CCN(CC(=O)O)CCN(CC(=O)O)CC2)cc1)C(=O)N1CCC[C@H]1B(O)O] | 1031 (M − H₂O + H) |
| | 2,2′,2″-(10-((4S,10S)-1-(4-(((R)-1-((R)-2-Boronopyrrolidin-1-yl)-1- | |
| | oxopropan-2-yl)carbamoyl)phenyl)-4-(carboxymethyl)-10-(4-guanidinobutyl)- | |
| | 3,6,9,12-tetraoxo-2,5,8,11-tetraazatridecan-13-yl)-1,4,7,10- | |
| | tetraazacyclododecane-1,4,7-triyl)triacetic acid | |
| 137 | 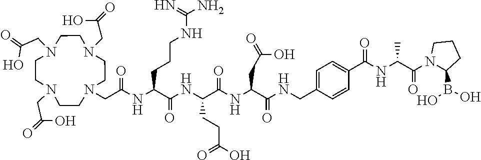 [SMILES: C[C@@H](NC(=O)c1ccc(CNC(=O)[C@H](CC(=O)O)NC(=O)[C@H](CCC(=O)O)NC(=O)[C@H](CCCNC(=N)N)NC(=O)CN2CCN(CC(=O)O)CCN(CC(=O)O)CCN(CC(=O)O)CC2)cc1)C(=O)N1CCC[C@H]1B(O)O] | 1088 (M − H₂O + H) |
| | 2,2′,2″-(10-((4S,7S,10S)-1-(4-(((R)-1-((R)-2-Boronopyrrolidin-1-yl)-1- | |
| | oxopropan-2-yl)carbamoyl)phenyl)-7-(2-carboxyethyl)-4-(carboxymethyl)-10- | |
| | (3-guanidinopropyl)-3,6,9,12-tetraoxo-2,5,8,11-tetraazatridecan-13-yl)- | |
| | 1,4,7,10-tetraazacyclododecane-1,4,7-triyl)triacetic acid | |
| 138 | 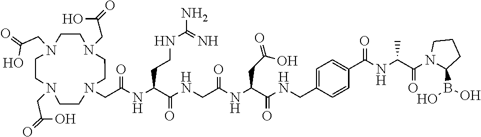 [SMILES: C[C@@H](NC(=O)c1ccc(CNC(=O)[C@H](CC(=O)O)NC(=O)CNC(=O)[C@H](CCNC(=N)N)NC(=O)CN2CCN(CC(=O)O)CCN(CC(=O)O)CCN(CC(=O)O)CC2)cc1)C(=O)N1CCC[C@H]1B(O)O] | 1002 (M − H₂O + H) |
| | 2,2′,2″-(10-((4S,10S)-1-(4-(((R)-1-((R)-2-Boronopyrrolidin-1-yl)-1- | |
| | oxopropan-2-yl)carbamoyl)phenyl)-4-(carboxymethyl)-10-(2-guanidinoethyl)- | |
| | 3,6,9,12-tetraoxo-2,5,8,11-tetraazatridecan-13-yl)-1,4,7,10- | |
| | tetraazacyclododecane-1,4,7-triyl)triacetic acid | |
| 139 | 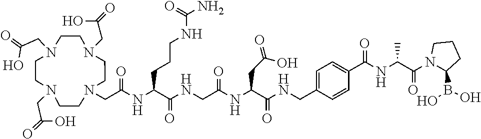 [SMILES: C[C@@H](NC(=O)c1ccc(CNC(=O)[C@H](CC(=O)O)NC(=O)CNC(=O)[C@H](CCCNC(N)=O)NC(=O)CN2CCN(CC(=O)O)CCN(CC(=O)O)CCN(CC(=O)O)CC2)cc1)C(=O)N1CCC[C@H]1B(O)O] | 1018 (M − H₂O + H) |
| | 2,2′,2″-(10-((4S,10S)-1-(4-(((R)-1-((R)-2-Boronopyrrolidin-1-yl)-1- | |
| | oxopropan-2-yl)carbamoyl)phenyl)-4-(carboxymethyl)-3,6,9,12-tetraoxo-10- | |
| | (3-ureidopropyl)-2,5,8,11-tetraazatridecan-13-yl)-1,4,7,10-acid | |
| | tetraazacyclododecane-1,4,7-triyl)triacetic | |

### Preparation 140

### 2,2′,2″-(10-((R)-4-(((S)-17-(4-(((R)-1-((R)-2-Boronopyrrolidin-1-yl)-1-oxopropan-2-yl)carbamoyl)phenyl)-3-imino-2-methyl-9,15-dioxo-2,4,10,16-tetraazaheptadecan-8-yl)amino)-1-carboxy-4-oxobutyl)-1,4,7,10-tetraazacyclododecane-1,4,7-triyl)triacetic acid

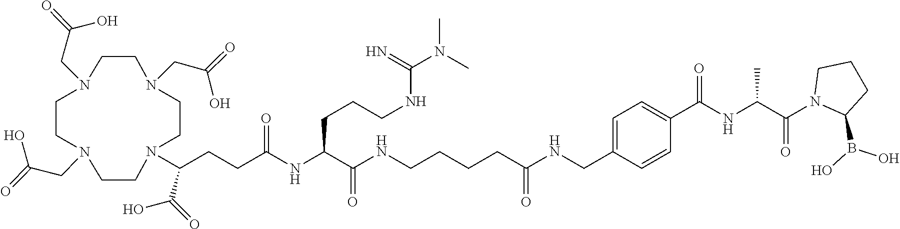 [SMILES: C[C@@H](NC(=O)c1ccc(CNC(=O)CCCCNC(=O)[C@H](CCCNC(=N)N(C)C)NC(=O)CC[C@H](C(=O)O)N2CCN(CC(=O)O)CCN(CC(=O)O)CCN(CC(=O)O)CC2)cc1)C(=O)N1CCC[C@H]1B(O)O]

4-((S)-8-Amino-3-imino-2-methyl-9,15-dioxo-2,4,10,16-tetraazaheptadecan-17-yl)-N-((2R)-1-oxo-1-((2R)-2-((3aS,4S,6S)-3a,5,5-trimethylhexahydro-4,6-methanobenzo[d][1,3,2]dioxaborol-2-yl)pyrrolidin-1-yl)propan-2-yl)benzamide was coupled with (R)-2,2′,2″-(10-(1-carboxy-4-(4-nitrophenoxy)-4-oxobutyl)-1,4,7,10-tetraazacyclododecane-1,4,7-triyl)triacetic acid in anhydrous DMF and TEA. The pinanediol protective group was removed with PhB(OH)2 in 0.01N HCl and MTBE (1:3) to afford the title compound (6 mg, 42%) as a white powder. ESI/MS (m/z): 1044 (M−H₂O+H).

### Preparation 141

### 2,2′-(7-((4S,10S,15R)-1-(4-(((R)-1-((R)-2-Boronopyrrolidin-1-yl)-1-oxopropan-2-yl)carbamoyl)phenyl)-15-carboxy-4-(carboxymethyl)-10-(3-guanidinopropyl)-3,6,9,12-tetraoxo-2,5,8,11-tetraazapentadecan-15-yl)-1,4,7-triazonane-1,4-diyl)diacetic acid

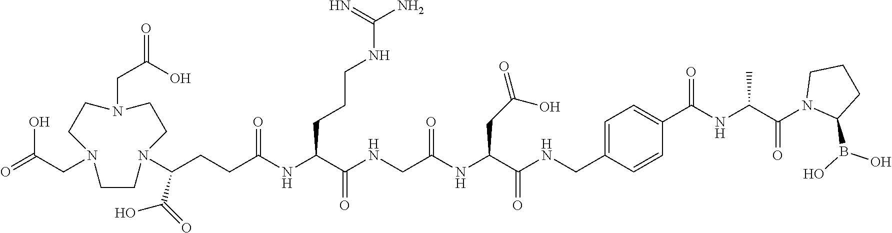 [SMILES: C[C@@H](NC(=O)c1ccc(CNC(=O)[C@H](CC(=O)O)NC(=O)CNC(=O)[C@H](CCCNC(=N)N)NC(=O)CC[C@H](C(=O)O)N2CCN(CC(=O)O)CCN(CC(=O)O)CC2)cc1)C(=O)N1CCC[C@H]1B(O)O]

tert-Butyl (3S)-3-(2-((S)-2-amino-5-(3-((2,2,4,6,7-pentamethyl-2,3-dihydrobenzofuran-5-yl)sulfonyl)guanidino)pentanamido)acetamido)-4-oxo-4-((4-(((2R)-1-oxo-1-((2R)-2-((3aS,4S,6S)-3a,5,5-trimethylhexahydro-4,6-methanobenzo[d][1,3,2]dioxaborol-2-yl)pyrrolidin-1-yl)propan-2-yl)carbamoyl)benzyl)amino)butanoate was coupled with (R)-4-(4,7-bis(2-(tert-butoxy)-2-oxoethyl)-1,4,7-triazonan-1-yl)-5-(tert-butoxy)-5-oxopentanoic acid according to SP-2. The —Pbf, -OtBu, and pinanediol protecting groups were then removed according to SP-6 to afford the title product (36.5 mg, 60%) as a white powder. ESI/MS (m/z): 987 (M−H₂O+H).

### Preparation 142

### 2,2′-(7-((R)-4-(((S)-17-(4-(((R)-1-((R)-2-Boronopyrrolidin-1-yl)-1-oxopropan-2-yl)carbamoyl)phenyl)-3-imino-2-methyl-9,15-dioxo-2,4,10,16-tetraazaheptadecan-8-yl)amino)-1-carboxy-4-oxobutyl)-1,4,7-triazonane-1,4-diyl)diacetic acid

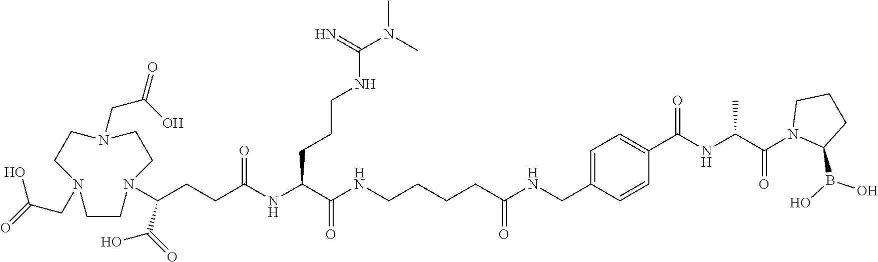 [SMILES: C[C@@H](NC(=O)c1ccc(CNC(=O)CCCCNC(=O)[C@H](CCCNC(=N)N(C)C)NC(=O)CC[C@H](C(=O)O)N2CCN(CC(=O)O)CCN(CC(=O)O)CC2)cc1)C(=O)N1CCC[C@H]1B(O)O]

4-((S,Z)-8-Amino-2-methyl-9,15-dioxo-3-((2,2,4,6,7-pentamethyl-2,3-dihydrobenzofuran)-5-sulfonamido)-2,4,10,16-tetraazaheptadec-3-en-17-yl)-N-((2R)-1-oxo-1-((2R)-2-((3aS,4S,6S)-3a,5,5-trimethylhexahydro-4,6-methanobenzo[d][1,3,2]dioxaborol-2-yl)pyrrolidin-1-yl)propan-2-yl)benzamide was coupled with (R)-4-(4,7-bis(2-(tert-butoxy)-2-oxoethyl)-1,4,7-triazonan-1-yl)-5-(tert-butoxy)-5-oxopentanoic acid with HATU and DIPEA in DMF according to SP-5. All the protecting groups were removed according to SP-6 to afford the title compound (30 mg, 46%) as a white powder. ESI/MS (m/z): 943 (M−H₂O+H).

The following compounds in Table 14 were synthesized in essentially the same manner as described for 2,2′-(7-((R)-4-(((S)-17-(4-(((R)-1-((R)-2-boronopyrrolidin-1-yl)-1-oxopropan-2-yl)carbamoyl)phenyl)-3-imino-2-methyl-9,15-dioxo-2,4,10,16-tetraazaheptadecan-8-yl)amino)-1-carboxy-4-oxobutyl)-1,4,7-triazonane-1,4-diyl)diacetic acid.

TABLE 14

| | | ESI/MS |
|---|---|---|
| Prep # | Structure | m/z |
| 143 | 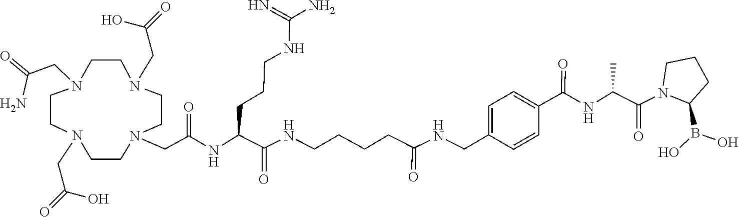 | 943 (M − H₂O + H) |
| | 2,2′-(4-(2-Amino-2-oxoethyl)-10-(2-(((S)-1-((5-((4-(((R)-1-((R)-2- | |
| | boronopyrrolidin-1-yl)-1-oxopropan-2-yl)carbamoyl)benzyl)amino)-5- | |
| | oxopentyl)amino)-5-guanidino-1-oxopentan-2-yl)amino)-2-oxoethyl)-1,4,7,10- | |
| | tetraazacyclododecane-1,7-diyl)diacetic acid | |
| 144 | 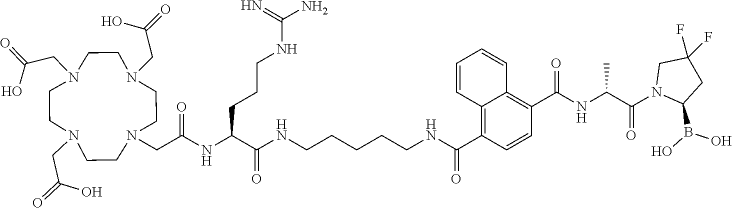 [SMILES: C[C@@H](NC(=O)c1ccc(C(=O)NCCCCCNC(=O)[C@H](CCCNC(=N)N)NC(=O)CN2CCN(CC(=O)O)CCN(CC(=O)O)CCN(CC(=O)O)CC2)c2ccccc12)C(=O)N1CC(F)(F)C[C@H]1B(O)O] | 1029 (M − H₂O + H) |
| | 2,2′,2″-(10-(2-(((S)-1-((5-(4-(((R)-1-((R)-2-Borono-4,4-difluoropyrrolidin-1- | |
| | yl)-1-oxopropan-2-yl)carbamoyl)-1-naphthamido)pentyl)amino)-5-guanidino- | |
| | 1-oxopentan-2-yl)amino)-2-oxoethyl)-1,4,7,10-tetraazacyclododecane-1,4,7- | |
| | triyl)triacetic acid | |
| 145 | 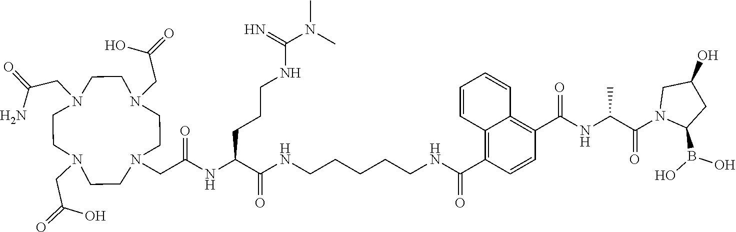 [SMILES: C[C@@H](NC(=O)c1ccc(C(=O)NCCCCCNC(=O)[C@H](CCCNC(=N)N(C)C)NC(=O)CN2CCN(CC(=O)O)CCN(CC(N)=O)CCN(CC(=O)O)CC2)c2ccccc12)C(=O)N1C[C@@H](O)C[C@H]1B(O)O] | 1037 (M − H₂O + H) |
| | 2,2′-(4-(2-Amino-2-oxoethyl)-10-(2-(((S)-17-(4-(((R)-1-((2R,4S)-2-borono-4- | |
| | hydroxypyrrolidin-1-yl)-1-oxopropan-2-yl)carbamoyl)naphthalen-1-yl)-3- | |
| | imino-2-methyl-9,17-dioxo-2,4,10,16-tetraazaheptadecan-8-yl)amino)-2- | |
| | oxoethyl)-1,4,7,10-tetraazacyclododecane-1,7-diyl)diacetic acid | |
| 146 | 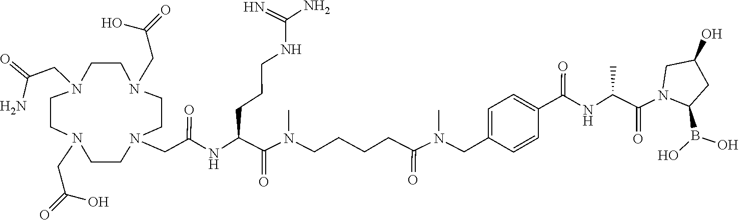 | 987 (M − H₂O + H) |
| | 2,2′-(4-(2-Amino-2-oxoethyl)-10-(2-(((S)-1-((5-((4-(((R)-1-((2R,4S)-2- | |
| | borono-4-hydroxypyrrolidin-1-yl)-1-oxopropan-2- | |
| | yl)carbamoyl)benzyl)(methyl)amino)-5-oxopentyl)(methyl)amino)-5- | |
| | guanidino-1-oxopentan-2-yl)amino)-2-oxoethyl)-1,4,7,10- | |
| | tetraazacyclododecane-1,7-diyl)diacetic acid | |
| 147 | 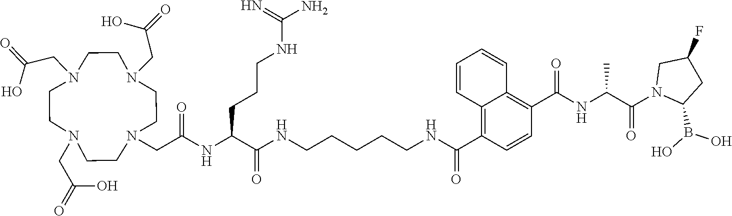 [SMILES: C[C@@H](NC(=O)c1ccc(C(=O)NCCCCCNC(=O)[C@H](CCCNC(=N)N)NC(=O)CN2CCN(CC(=O)O)CCN(CC(=O)O)CCN(CC(=O)O)CC2)c2ccccc12)C(=O)N1C[C@@H](F)C[C@@H]1B(O)O] | 1012 (M − H₂O + H) |
| | 2,2′,2″-(10-(2-(((S)-1-((5-(4-(((R)-1-((2S,4S)-2-Borono-4-fluoropyrrolidin-1- | |
| | yl)-1-oxopropan-2-yl)carbamoyl)-1-naphthamido)pentyl)amino)-5-guanidino- | |
| | 1-oxopentan-2-yl)amino)-2-oxoethyl)-1,4,7,10-tetraazacyclododecane-1,4,7- | |
| | triyl)triacetic acid | |

### Preparation 149

### 2,2′,2″-(10-((10S,20R,29R)-1-(4-(((R)-1-((R)-2-Boronopyrrolidin-1-yl)-1-oxopropan-2-yl)carbamoyl)phenyl)-20,29-dicarboxy-10-(3-guanidinopropyl)-3,9,12,15,18,26-hexaoxo-2,8,11,16,19,25-hexaazanonacosan-29-yl)-1,4,7,10-tetraazacyclododecane-1,4,7-triyl)triacetic acid

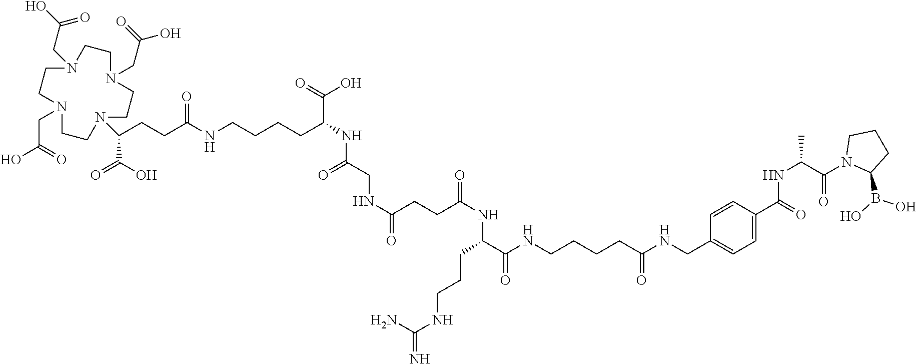 [SMILES: C[C@@H](NC(=O)c1ccc(CNC(=O)CCCCNC(=O)[C@H](CCCNC(=N)N)NC(=O)CCC(=O)NCC(=O)N[C@H](CCCCNC(=O)CC[C@H](C(=O)O)N2CCN(CC(=O)O)CCN(CC(=O)O)CCN(CC(=O)O)CC2)C(=O)O)cc1)C(=O)N1CCC[C@H]1B(O)O]

The initial resin loading with N2-(((9H-fluoren-9-yl)methoxy)carbonyl)-N6-(tert-butoxycarbonyl)-L-lysine was performed as described in SP-1. The Fmoc protecting group was removed according to SP-3 and then coupled with (((9H-fluoren-9-yl)methoxy)carbonyl)glycine according to SP-2. The Fmoc protecting group was removed according to SP-3 and then coupled with 4-oxo-4-(((2S)-1-oxo-1-((5-oxo-5-((4-(((2R)-1-oxo-1-((2R)-2-((3aS,4S,6S)-3a,5,5-trimethylhexahydro-4,6-methanobenzo[d][1,3,2]dioxaborol-2-yl)pyrrolidin-1-yl)propan-2-yl)carbamoyl)benzyl)amino)pentyl)amino)-5-(3-((2,2,4,5,7-pentamethyl-2,3-dihydrobenzofuran-6-yl)sulfonyl)guanidino)pentan-2-yl)amino)butanoic acid according to SP-2 using PyBrop as the coupling reagent. The peptide was cleaved from the resin according to SP-4 (4N HCl in 1,4-dioxane was added here to ensure the Boc protection at Lys was completely removed) and was conjugated with (R)-2,2′,2″-(10-(1-carboxy-4-(4-nitrophenoxy)-4-oxobutyl)-1,4,7,10-tetraazacyclododecane-1,4,7-triyl)triacetic acid in anhydrous DMF and DIPEA. All the protecting groups were removed according to SP-6 (in this case, the removal of pinanediol needed additional treatment with PhB(OH)₂ to be removed completely) to afford the title compound (13 mg, 16%) as a white powder. ESI/MS (m/z): 1301 (M−H₂O+H).

### Preparation 150

### 2,2′-(7-((10S,20S,29R)-1-(4-(((R)-1-((R)-2-Boronopyrrolidin-1-yl)-1-oxopropan-2-yl)carbamoyl)naphthalen-1-yl)-20,29-dicarboxy-10-(3-guanidinopropyl)-1,9,12,18,26-pentaoxo-2,8,11,19,25-pentaazanonacosan-29-yl)-1,4,7-triazonane-1,4-diyl)diacetic acid

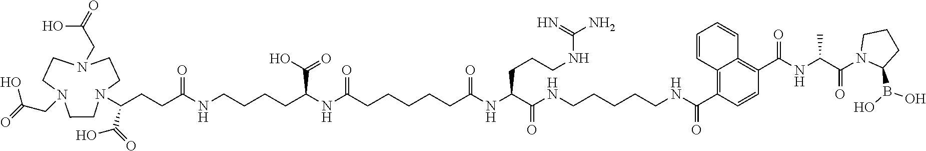 [SMILES: C[C@@H](NC(=O)c1ccc(C(=O)NCCCCCNC(=O)[C@H](CCCNC(=N)N)NC(=O)CCCCCC(=O)N[C@@H](CCCCNC(=O)CC[C@H](C(=O)O)N2CCN(CC(=O)O)CCN(CC(=O)O)CC2)C(=O)O)c2ccccc12)C(=O)N1CCC[C@H]1B(O)O]

(S)-4-((5-(2-(7-(tert-Butoxy)-7-oxoheptanamido)-5-(3-((2,2,4,6,7-pentamethyl-2,3-dihydrobenzofuran-5-yl)sulfonyl)guanidino)pentanamido)pentyl)carbamoyl)-1-naphthoic acid was coupled with (2R)-2-amino-1-((2R)-2-((3aS,4S,6S)-3a,5,5-trimethylhexahydro-4,6-methanobenzo[d][1,3,2]dioxaborol-2-yl)pyrrolidin-1-yl)propan-1-one hydrochloride according to SP-5. The -tBu group was removed with 4N HCl in 1,4-dioxane and then coupled with tert-butyl N6-(((9H-fluoren-9-yl)methoxy)carbonyl)-L-lysinate according to SP-5. The Fmoc protecting group was removed with piperidine (5%) in DMF and was then coupled to (R)-4-(4,7-bis(2-(tert-butoxy)-2-oxoethyl)-1,4,7-triazonan-1-yl)-5-(tert-butoxy)-5-oxopentanoic acid according to SP-5 except using PyBrop instead of HATU. All the protecting groups were removed according to SP-6 to afford the title product (16 mg, 20%) as a white powder. ESI/MS (m/z): 1234 (M−H₂O+H).

### Preparation 151

### 2,2′,2″-(10-(2-(((S)-17-(4-(((R)-1-((2R,4S)-2-Borono-4-hydroxypyrrolidin-1-yl)-1-oxopropan-2-yl)carbamoyl)naphthalen-1-yl)-3-imino-2,10-dimethyl-9,17-dioxo-2,4,10,16-tetraazaheptadecan-8-yl)amino)-2-oxoethyl)-1,4,7,10-tetraazacyclododecane-1,4,7-triyl)triacetic acid

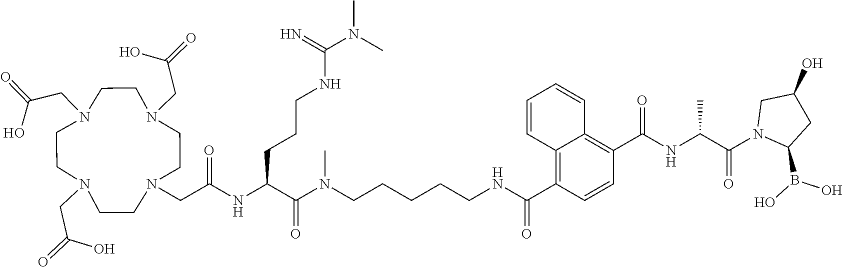 [SMILES: C[C@@H](NC(=O)c1ccc(C(=O)NCCCCCN(C)C(=O)[C@H](CCCNC(=N)N(C)C)NC(=O)CN2CCN(CC(=O)O)CCN(CC(=O)O)CCN(CC(=O)O)CC2)c2ccccc12)C(=O)N1C[C@@H](O)C[C@H]1B(O)O]

The initial resin loading with naphthalene-1,4-dicarboxylic acid was performed according to SP-1 and was coupled with (9H-fluoren-9-yl)methyl (S,E)-(1-((5-aminopentyl)(methyl)amino)-5-(((dimethylamino)((2,2,4,6,7-pentamethyl-2,3-dihydrobenzofuran)-5-sulfonamido)methylene)amino)-1-oxopentan-2-yl)carbamate hydrochloride according to SP-2 except with PyBrop as the coupling reagent. The Fmoc protecting group was removed according to SP-3 and then coupled with 2-(7-(2-amino-2-oxoethyl)-4,10-bis(2-(tert-butoxy)-2-oxoethyl)-1,4,7,10-tetraazacyclododecan-1-yl)acetic acid according to SP-2. The peptide was cleaved from the resin according to SP-4 and was coupled with (2R)-2-amino-1-((2R,4S)-4-hydroxy-2-((3aS,4S,6S)-3a,5,5-trimethylhexahydro-4,6-methanobenzo[d][1,3,2]dioxaborol-2-yl)pyrrolidin-1-yl)propan-1-one hydrochloride according to SP-5. All the protecting groups were removed according to SP-6 to afford the title compound (9.3 mg, 13%). ESI/MS (m/z): 1052 (M−H₂O+H).

### Preparation 152

### 2,2′-(4-(2-Amino-2-oxoethyl)-10-(2-(((S)-1-(4-(2-(4-(((R)-1-((2R,4S)-2-borono-4-hydroxypyrrolidin-1-yl)-1-oxopropan-2-yl)carbamoyl)-1-naphthamido)ethyl)piperazin-1-yl)-5-(3,3-dimethylguanidino)-1-oxopentan-2-yl)amino)-2-oxoethyl)-1,4,7,10-tetraazacyclododecane-1,7-diyl)diacetic acid

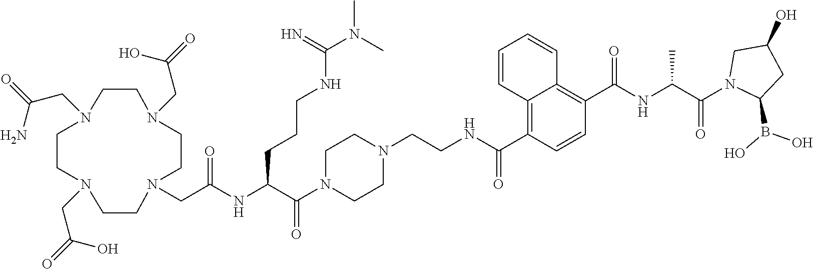 [SMILES: C[C@@H](NC(=O)c1ccc(C(=O)NCCN2CCN(C(=O)[C@H](CCCNC(=N)N(C)C)NC(=O)CN3CCN(CC(=O)O)CCN(CC(N)=O)CCN(CC(=O)O)CC3)CC2)c2ccccc12)C(=O)N1C[C@@H](O)C[C@H]1B(O)O]

The initial resin loading with naphthalene-1,4-dicarboxylic acid was performed according to SP-1 and was coupled with (9H-fluoren-9-yl)methyl (S,E)-(1-(4-(2-aminoethyl)piperazin-1-yl)-5-(((dimethylamino)((2,2,4,6,7-pentamethyl-2,3-dihydrobenzofuran)-5-sulfonamido)methylene)amino)-1-oxopentan-2-yl)carbamate hydrochloride according to SP-2 except with PyBrop as the coupling reagent. The Fmoc protecting group was removed according to SP-3 and was coupled with 2-(7-(2-amino-2-oxoethyl)-4,10-bis(2-(tert-butoxy)-2-oxoethyl)-1,4,7,10-tetraazacyclododecan-1-yl)acetic acid according to SP-2. The peptide was cleaved from the resin according to SP-4 and was coupled with (2R)-2-amino-1-((2R,4S)-4-hydroxy-2-((3aS,4S,6S)-3a,5,5-trimethylhexahydro-4,6-methanobenzo[d][1,3,2]dioxaborol-2-yl)pyrrolidin-1-yl)propan-1-one hydrochloride according to SP-5. All the protecting groups were removed according to SP-6 to afford the title product (35.9 mg, 23%). ESI/MS (m/z): 1064 (M−H₂O+H).

The following compounds in Table 16 were synthesized in essentially the same manner as described for 2,2′-(4-(2-amino-2-oxoethyl)-10-(2-(((S)-1-(4-(2-(4-(((R)-1-((2R,4S)-2-borono-4-hydroxypyrrolidin-1-yl)-1-oxopropan-2-yl)carbamoyl)-1-naphthamido)ethyl)piperazin-1-yl)-5-(3,3-dimethylguanidino)-1-oxopentan-2-yl)amino)-2-oxoethyl)-1,4,7,10-tetraazacyclododecane-1,7-diyl)diacetic acid.

TABLE 16

| | | ESI/MS |
|---|---|---|
| Prep # | Structure | m/z |
| 153 | 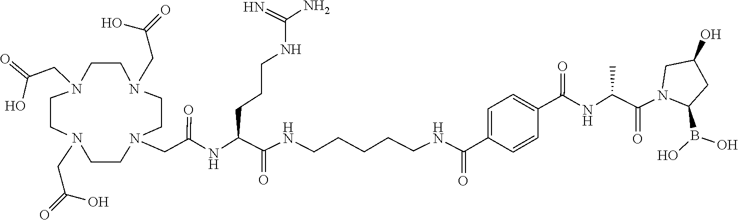 | 959 (M − H₂O + H) |
| | 2,2′,2″-(10-(2-(((S)-1-((5-(4-(((R)-1-((2R,4S)-2-Borono-4-hydroxypyrrolidin- | |
| | 1-yl)-1-oxopropan-2-yl)carbamoyl)benzamido)pentyl)amino)-5-guanidino-1- | |
| | oxopentan-2-yl)amino)-2-oxoethyl)-1,4,7,10-tetraazacyclododecane-1,4,7- | |
| | triyl)triacetic acid | |
| 154 | 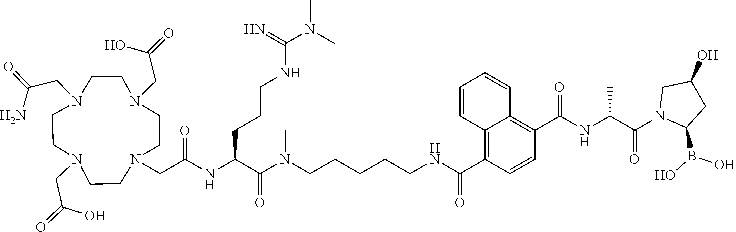 [SMILES: C[C@@H](NC(=O)c1ccc(C(=O)NCCCCCN(C)C(=O)[C@H](CCCNC(=N)N(C)C)NC(=O)CN2CCN(CC(=O)O)CCN(CC(N)=O)CCN(CC(=O)O)CC2)c2ccccc12)C(=O)N1C[C@@H](O)C[C@H]1B(O)O] | 1051 (M − H₂O + H) |
| | 2,2′-(4-(2-Amino-2-oxoethyl)-10-(2-(((S)-17-(4-(((R)-1-((2R,4S)-2-borono-4- | |
| | hydroxypyrrolidin-1-yl)-1-oxopropan-2-yl)carbamoyl)naphthalen-1-yl)-3- | |
| | imino-2,10-dimethyl-9,17-dioxo-2,4,10,16-tetraazaheptadecan-8-yl)amino)-2- | |
| | oxoethyl)-1,4,7,10-tetraazacyclododecane-1,7-diyl)diacetic acid | |

### Preparation 155

### 2,2′-(7-((10S,29R)-1-(4-(((R)-1-((2R,4S)-2-Borono-4-hydroxypyrrolidin-1-yl)-1-oxopropan-2-yl)carbamoyl)phenyl)-29-carboxy-10-(3-(3,3-dimethylguanidino)propyl)-3,9,12,18,26-pentaoxo-2,8,11,19,25-pentaazanonacosan-29-yl)-1,4,7-triazonane-1,4-diyl)diacetic acid

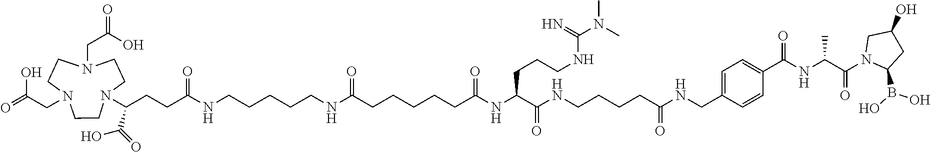 [SMILES: C[C@@H](NC(=O)c1ccc(CNC(=O)CCCCNC(=O)[C@H](CCCNC(=N)N(C)C)NC(=O)CCCCCC(=O)NCCCCCNC(=O)CC[C@H](C(=O)O)N2CCN(CC(=O)O)CCN(CC(=O)O)CC2)cc1)C(=O)N1C[C@@H](O)C[C@H]1B(O)O]

The initial resin loading with 4-(((((9H-fluoren-9-yl)methoxy)carbonyl)amino)methyl)benzoic acid was performed as described in SP-1. The Fmoc protecting group was removed according to SP-3 and was then coupled with 5-((((9H-fluoren-9-yl)methoxy)carbonyl)amino)pentanoic acid according to SP-2 using PyBrop as the coupling reagent. The Fmoc group was removed according to SP-3 and was coupled with (E)-N2-(((9H-fluoren-9-yl)methoxy)carbonyl)-N,N-dimethyl-N′-((2,2,4,6,7-pentamethyl-2,3-dihydrobenzofuran-5-yl)sulfonyl)-L-arginine according to SP-2 using PyBrop as the coupling reagent. The Fmoc protecting group was removed according to SP-3 and was coupled with 7-(4-nitrophenoxyl)-7-oxoheptanoic acid according to SP-2 without the addition of HATU. The product was coupled with (9H-fluoren-9-yl)methyl (5-aminopentyl)carbamate HCl according to SP-2 using PyBrop as the coupling reagent. The Fmoc protecting group was removed according to SP-3 and was coupled with (R)-4-(4,7-bis(2-(tert-butoxy)-2-oxoethyl)-1,4,7-triazonan-1-yl)-5-(tert-butoxy)-5-oxopentanoic acid according to SP-2 using PyBrop as the coupling reagent. The peptide was cleaved from the resin according to SP-4 and was coupled with (2R)-2-amino-1-((2R,4S)-4-hydroxy-2-((3aS,4S,6S)-3a,5,5-trimethylhexahydro-4,6-methanobenzo[d][1,3,2]dioxaborol-2-yl)pyrrolidin-1-yl)propan-1-one hydrochloride according to SP-5. All the protecting groups were removed according to SP-6 to afford the title product (20.7 mg, 13%). ESI/MS (m/z): 1185 (M−H₂O+H).

### Preparation 156

### 2,2′-(7-((R)-4-(((S)-17-((1R,4S)-4-(((R)-1-((2R,4S)-2-Borono-4-hydroxypyrrolidin-1-yl)-1-oxopropan-2-yl)carbamoyl)cyclohexyl)-3-imino-2-methyl-9,15-dioxo-2,4,10,16-tetraazaheptadecan-8-yl)amino)-1-carboxy-4-oxobutyl)-1,4,7-triazonane-1,4-diyl)diacetic acid

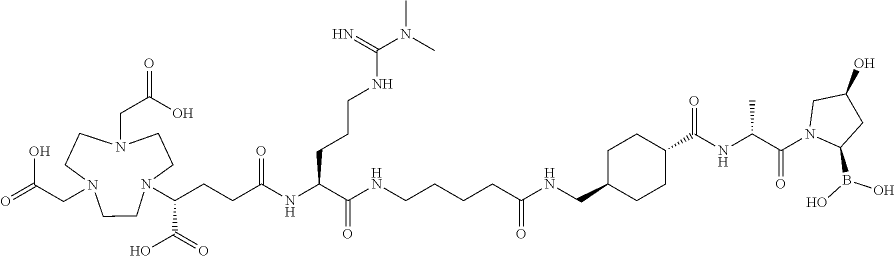 [SMILES: C[C@@H](NC(=O)[C@H]1CC[C@H](CNC(=O)CCCCNC(=O)[C@H](CCCNC(=N)N(C)C)NC(=O)CC[C@H](C(=O)O)N2CCN(CC(=O)O)CCN(CC(=O)O)CC2)CC1)C(=O)N1C[C@@H](O)C[C@H]1B(O)O]

The initial resin loading with (1r,4r)-4-(((((9H-fluoren-9-yl)methoxy)carbonyl)amino)methyl)cyclohexane-1-carboxylic acid was performed as described in SP-1. The Fmoc protecting group was removed according to SP-3 and was coupled with 5-((((9H-fluoren-9-yl)methoxy)carbonyl)amino)pentanoic acid according to SP-2 using PyBrop as the coupling reagent. The Fmoc protecting group was removed according to SP-3 and was coupled with (E)-N²-(((9H-fluoren-9-yl)methoxy)carbonyl)-N^{w},N^{w}-dimethyl-N^{w′}-((2,2,4,6,7-pentamethyl-2,3-dihydrobenzofuran-5-yl)sulfonyl)-L-arginine according to SP-2 using PyBrop as the coupling reagent. The Fmoc protecting group was removed according to SP-3 and was coupled with (R)-4-(4,7-bis(2-(tert-butoxy)-2-oxoethyl)-1,4,7-triazonan-1-yl)-5-(tert-butoxy)-5-oxopentanoic acid according to SP-2 using PyBrop as the coupling reagent. The peptide was cleaved from the resin according to SP-4 and was coupled with (2R)-2-amino-1-((2R,4S)-4-hydroxy-2-((3aS,4S,6S)-3a,5,5-trimethylhexahydro-4,6-methanobenzo[d][1,3,2]dioxaborol-2-yl)pyrrolidin-1-yl)propan-1-one hydrochloride according to SP-5. All the protecting groups were removed according to SP-6 to afford the title product (27.7 mg, 21%). ESI/MS (m/z): 965 (M−H₂O+H).

The following compounds in Table 17 were synthesized in essentially the same manner as described for 2,2′-(7-((R)-4-(((S)-17-((1R,4S)-4-(((R)-1-((2R,4S)-2-borono-4-hydroxypyrrolidin-1-yl)-1-oxopropan-2-yl)carbamoyl)cyclohexyl)-3-imino-2-methyl-9,15-dioxo-2,4,10,16-tetraazaheptadecan-8-yl)amino)-1-carboxy-4-oxobutyl)-1,4,7-triazonane-1,4-diyl)diacetic acid.

TABLE 17

| | | ESI/MS |
|---|---|---|
| Prep # | Structure | m/z |
| 157 | 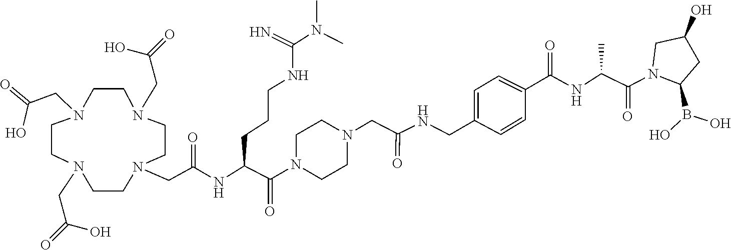 | 1015 (M − H₂O + H) |
| | 2,2′,2″-(10-(2-(((S)-1-(4-(2-((4-(((R)-1-((2R,4S)-2-Borono-4- | |
| | hydroxypyrrolidin-1-yl)-1-oxopropan-2-yl)carbamoyl)benzyl)amino)-2- | |
| | oxoethyl)piperazin-1-yl)-5-(3,3-dimethylguanidino)-1-oxopentan-2-yl)amino)- | |
| | 2-oxoethyl)-1,4,7,10-tetraazacyclododecane-1,4,7-triyl)triacetic acid | |
| 158 | 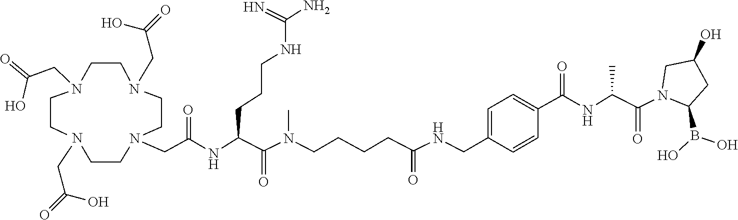 | 487 (M − H₂O/2 + H) |
| | 2,2′,2″-(10-(2-(((S)-1-((5-((4-(((R)-1-((2R,4S)-2-Borono-4-hydroxypyrrolidin- | |
| | 1-yl)-1-oxopropan-2-yl)carbamoyl)benzyl)amino)-5- | |
| | oxopentyl)(methyl)amino)-5-guanidino-1-oxopentan-2-yl)amino)-2-oxoethyl)- | |
| | 1,4,7,10-tetraazacyclododecane-1,4,7-triyl)triacetic acid | |
| 159 | 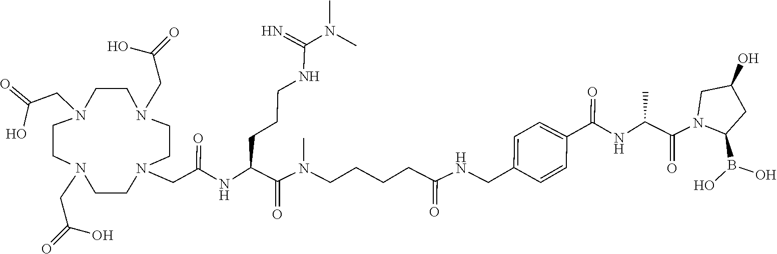 | 1002 (M − H₂O + H) |
| | 2,2′,2″-(10-(2-(((S)-17-(4-(((R)-1-((2R,4S)-2-Borono-4-hydroxypyrrolidin-1- | |
| | yl)-1-oxopropan-2-yl)carbamoyl)phenyl)-3-imino-2,10-dimethyl-9,15-dioxo- | |
| | 2,4,10,16-tetraazaheptadecan-8-yl)amino)-2-oxoethyl)-1,4,7,10- | |
| | tetraazacyclododecane-1,4,7-triyl)triacetic acid | |
| 160 | 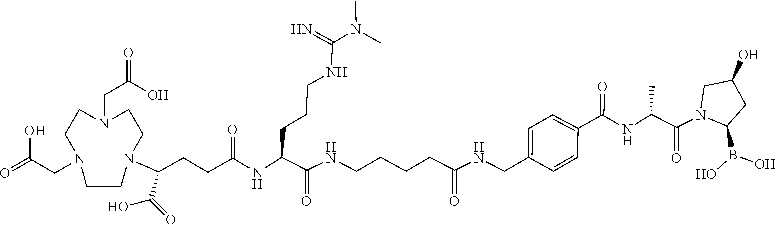 [SMILES: C[C@@H](NC(=O)c1ccc(CNC(=O)CCCCNC(=O)[C@H](CCCNC(=N)N(C)C)NC(=O)CC[C@H](C(=O)O)N2CCN(CC(=O)O)CCN(CC(=O)O)CC2)cc1)C(=O)N1C[C@@H](O)C[C@H]1B(O)O] | 958 (M − H₂O + H) |
| | 2,2′-(7-((R)-4-(((S)-17-(4-(((R)-1-((2R,4S)-2-Borono-4-hydroxypyrrolidin-1- | |
| | yl)-1-oxopropan-2-yl)carbamoyl)phenyl)-3-imino-2-methyl-9,15-dioxo- | |
| | 2,4,10,16-tetraazaheptadecan-8-yl)amino)-1-carboxy-4-oxobutyl)-1,4,7- | |
| | triazonane-1,4-diyl)diacetic acid | |
| 161 | 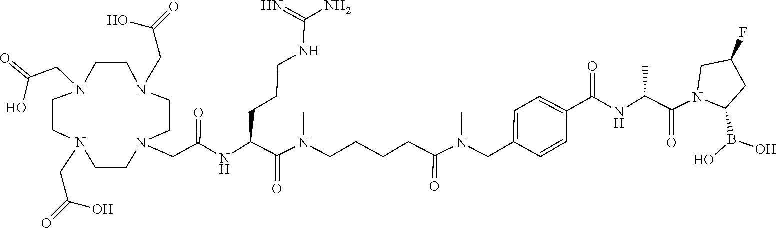 | 990 (M − H₂O + H) |
| | 2,2′,2″-(10-(2-(((S)-1-((5-((4-(((R)-1-((2S,4S)-2-Borono-4-fluoropyrrolidin-1- | |
| | yl)-1-oxopropan-2-yl)carbamoyl)benzyl)(methyl)amino)-5- | |
| | oxopentyl)(methyl)amino)-5-guanidino-1-oxopentan-2-yl)amino)-2-oxoethyl)- | |
| | 1,4,7,10-tetraazacyclododecane-1,4,7-triyl)triacetic acid | |
| 162 | 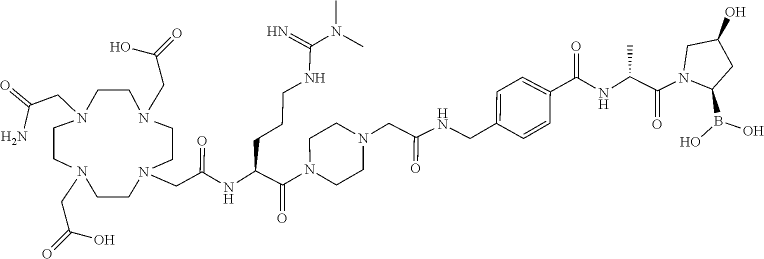 | 1014 (M − H₂O + H) |
| | 2,2′-(4-(2-Amino-2-oxoethyl)-10-(2-(((S)-1-(4-(2-((4-(((R)-1-((2R,4S)-2- | |
| | borono-4-hydroxypyrrolidin-1-yl)-1-oxopropan-2-yl)carbamoyl)benzyl)amino)- | |
| | 2-oxoethyl)piperazin-1-yl)-5-(3,3-dimethylguanidino)-1-oxopentan-2- | |
| | yl)amino)-2-oxoethyl)-1,4,7,10-tetraazacyclododecane-1,7-diyl)diacetic acid | |

### Preparation 163

### 2,2′-(7-((R)-4-(((S)-1-((5-(4-(((R)-1-((R)-2-Boronopyrrolidin-1-yl)-1-oxopropan-2-yl)carbamoyl)-1-naphthamido)pentyl)amino)-5-guanidino-1-oxopentan-2-yl)amino)-1-carboxy-4-oxobutyl)-1,4,7-triazonane-1,4-diyl)diacetic acid

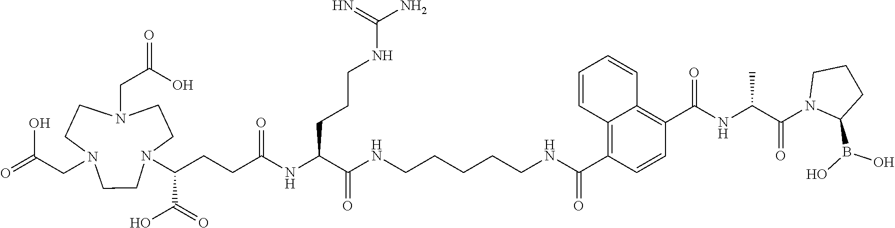 [SMILES: C[C@@H](NC(=O)c1ccc(C(=O)NCCCCCNC(=O)[C@H](CCCNC(=N)N)NC(=O)CC[C@H](C(=O)O)N2CCN(CC(=O)O)CCN(CC(=O)O)CC2)c2ccccc12)C(=O)N1CCC[C@H]1B(O)O]

The initial resin loading with naphthalene-1,4-dicarboxylic acid was performed as described in SP-1 and was coupled with (9H-fluoren-9-yl)methyl (5-aminopentyl)carbamate hydrochloride according to SP-2 using PyBrop as the coupling reagent. The Fmoc group was removed according to SP-3 and was coupled with N²-(((9H-fluoren-9-yl)methoxy)carbonyl)-N^{w}-((2,2,4,6,7-pentamethyl-2,3-dihydrobenzofuran-5-yl)sulfonyl)-L-arginine according to SP-2 using PyBrop as the coupling reagent. The Fmoc protecting group was removed according to SP-3 and was coupled with (R)-4-(4,7-bis(2-(tert-butoxy)-2-oxoethyl)-1,4,7-triazonan-1-yl)-5-(tert-butoxy)-5-oxopentanoic acid according to SP-2 using PyBrop as the coupling reagent. The peptide was cleaved from the resin according to SP-4 and was coupled with (2R)-2-amino-1-((2R)-2-((3aS,4S,6S)-3a,5,5-trimethylhexahydro-4,6-methanobenzo[d][1,3,2]dioxaborol-2-yl)pyrrolidin-1-yl)propan-1-one hydrochloride according to SP-5. All the protecting groups were removed according to SP-6 to afford the title compound (12.2 mg, 18%). ESI/MS (m/z): 965 (M−H₂O+H).

The following compounds in Table 18 were synthesized in essentially the same manner as described for 2,2′-(7-((R)-4-(((S)-1-((5-(4-(((R)-1-((R)-2-boronopyrrolidin-1-yl)-1-oxopropan-2-yl)carbamoyl)-1-naphthamido)pentyl)amino)-5-guanidino-1-oxopentan-2-yl)amino)-1-carboxy-4-oxobutyl)-1,4,7-triazonane-1,4-diyl)diacetic acid.

TABLE 18

| Prep | | ESI/MS |
|---|---|---|
| # | Structure | m/z |
| 164 | 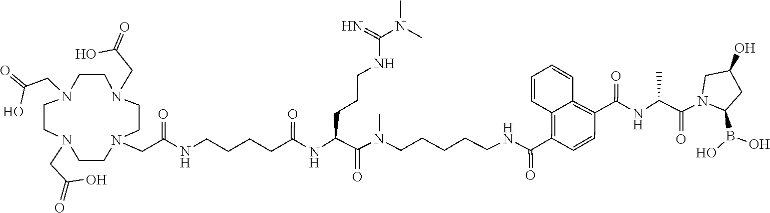 | 1151 (M − H₂O + H) |
| | 2,2′,2″-(10-((S)-1-(4-(((R)-1-((2R,4S)-2-Borono-4-hydroxypyrrolidin-1-yl)-1- | |
| | oxopropan-2-yl)carbamoyl)naphthalen-1-yl)-10-(3-(3,3- | |
| | dimethylguanidino)propyl)-8-methyl-1,9,12,18-tetraoxa-2,8,11,17- | |
| | tetraazanonadecan-19-yl)-1,4,7,10-tetraazacyclododecane-1,4,7-triyl)triacetic acid | |
| 165 | 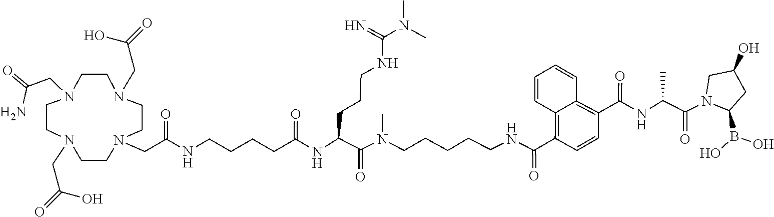 | 575 [(M − H₂O)/2 + H] |
| | 2,2′-(4-(2-Amino-2-oxoethyl)-10-((S)-1-(4-((R)-1-((2R,4S)-2-borono-4- | |
| | hydroxypyrrolidin-1-yl)-1-oxopropan-2-yl)carbamoyl)naphthalen-1-yl)-10-(3- | |
| | (3,3-dimethylguanidino)propyl)-8-methyl-1,9,12,18-tetraoxa-2,8,11,17- | |
| | tetraazanonadecan-19-yl)-1,4,7,10-tetraazacyclododecane-1,7-diyl)diacetic acid | |

### Preparation 166

### 2,2′-(4-(2-Amino-2-oxoethyl)-10-((4S,10S)-1-(4-(((R)-1-((2R,4S)-2-borono-4-hydroxypyrrolidin-1-yl)-1-oxopropan-2-yl)carbamoyl)phenyl)-4-(carboxymethyl)-10-(3-guanidinopropyl)-3,6,9,12-tetraoxo-2,5,8,11-tetraazatridecan-13-yl)-1,4,7,10-tetraazacyclododecane-1,7-diyl)diacetic acid

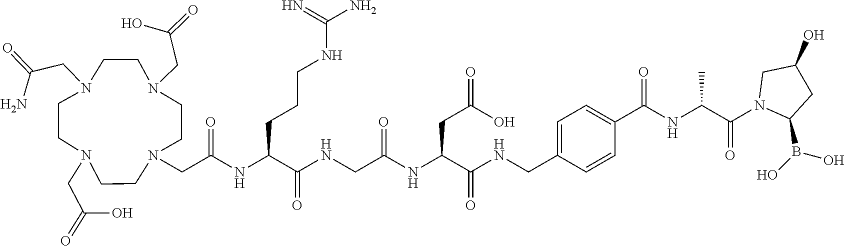 [SMILES: C[C@@H](NC(=O)c1ccc(CNC(=O)[C@H](CC(=O)O)NC(=O)CNC(=O)[C@H](CCCNC(=N)N)NC(=O)CN2CCN(CC(=O)O)CCN(CC(N)=O)CCN(CC(=O)O)CC2)cc1)C(=O)N1C[C@@H](O)C[C@H]1B(O)O]

The initial resin loading with 4-(((((9H-fluoren-9-yl)methoxy)carbonyl)amino)methyl)benzoic acid was performed according to SP-1. The Fmoc protecting group was removed according to SP-3 and was coupled with (S)-2-((((9H-fluoren-9-yl)methoxy)carbonyl)amino)-4-(tert-butoxy)-4-oxobutanoic acid according to SP-2. The Fmoc protecting group was removed according to SP-3 and was coupled with (((9H-fluoren-9-yl)methoxy)carbonyl)glycine according to SP-2. The Fmoc protecting group was removed according to SP-3 and was coupled with N²-(((9H-fluoren-9-yl)methoxy)carbonyl)-N^{w}-((2,2,4,6,7-pentamethyl-2,3-dihydrobenzofuran-5-yl)sulfonyl)-L-arginine according to SP-2 except with PyBrop as the coupling reagent. The Fmoc protecting group was removed according to SP-3 and was coupled to 2-(7-(2-amino-2-oxoethyl)-4,10-bis(2-(tert-butoxy)-2-oxoethyl)-1,4,7,10-tetraazacyclododecan-1-yl)acetic acid according to SP-2. The peptide was cleaved from the resin according to SP-4 and was coupled with (2R)-2-amino-1-((2R,4S)-4-hydroxy-2-((3aS,4S,6S)-3a,5,5-trimethylhexahydro-4,6-methanobenzo[d][1,3,2]dioxaborol-2-yl)pyrrolidin-1-yl)propan-1-one hydrochloride according to SP-5. All the protecting groups were removed according to SP-6 to afford the title compound (5.7 mg, 8%). ESI/MS (m/z): 1032 (M−H₂O+H).

The following compound in Table 19 was synthesized in essentially the same manner as described for 2,2′-(4-(2-amino-2-oxoethyl)-10-((4S,10S)-1-(4-(((R)-1-((2R,4S)-2-borono-4-hydroxypyrrolidin-1-yl)-1-oxopropan-2-yl)carbamoyl)phenyl)-4-(carboxymethyl)-10-(3-guanidinopropyl)-3,6,9,12-tetraoxo-2,5,8,11-tetraazatridecan-13-yl)-1,4,7,10-tetraazacyclododecane-1,7-diyl)diacetic acid.

TABLE 19

| | | ESI/MS |
|---|---|---|
| Prep # | Structure | m/z |
| 167 | 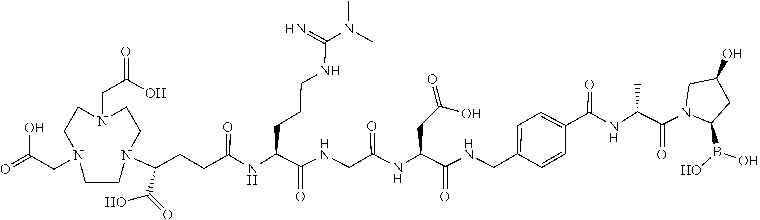 [SMILES: C[C@@H](NC(=O)c1ccc(CNC(=O)[C@H](CC(=O)O)NC(=O)CNC(=O)[C@H](CCCNC(=N)N(C)C)NC(=O)CC[C@H](C(=O)O)N2CCN(CC(=O)O)CCN(CC(=O)O)CC2)cc1)C(=O)N1C[C@@H](O)C[C@H]1B(O)O] | 1032 (M − H₂O + H) |
| | 2,2′-(7-((4S,10S,15R)-1-(4-(((R)-1-((2R,4S)-2-Borono-4-hydroxypyrrolidin-1- | |
| | yl)-1-oxopropan-2-yl)carbamoyl)phenyl)-15-carboxy-4-(carboxymethyl)-10-(3- | |
| | (3,3-dimethylguanidino)propyl)-3,6,9,12-tetraoxo-2,5,8,11-tetraazapentadecan- | |
| | 15-yl)-1,4,7-triazonane-1,4-diyl)diacetic acid | |

### Preparation 168

### 2,2′-(7-((10S,20S)-1-(4-(((R)-1-((R)-2-Boronopyrrolidin-1-yl)-1-oxopropan-2-yl)carbamoyl)naphthalen-1-yl)-20-carboxy-10-(3-guanidinopropyl)-1,9,12,18,26-pentaoxo-2,8,11,19,25-pentaazaheptacosan-27-yl)-1,4,7-triazonane-1,4-diyl)diacetic acid

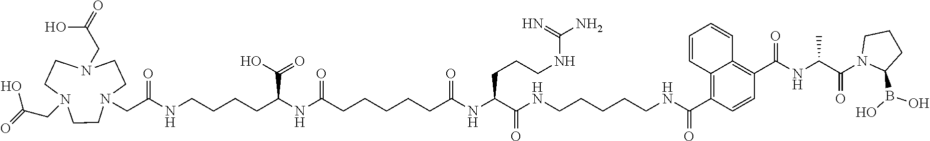 [SMILES: C[C@@H](NC(=O)c1ccc(C(=O)NCCCCCNC(=O)[C@H](CCCNC(=N)N)NC(=O)CCCCCC(=O)N[C@@H](CCCCNC(=O)CN2CCN(CC(=O)O)CCN(CC(=O)O)CC2)C(=O)O)c2ccccc12)C(=O)N1CCC[C@H]1B(O)O]

(S)-4-((5-(2-(7-(tert-Butoxy)-7-oxoheptanamido)-5-(3-((2,2,4,6,7-pentamethyl-2,3-dihydrobenzofuran-5-yl)sulfonyl)guanidino)pentanamido)pentyl)carbamoyl)-1-naphthoic acid was coupled with (2R)-2-amino-1-((2R)-2-((3aS,4S,6S)-3a,5,5-trimethylhexahydro-4,6-methanobenzo[d][1,3,2]dioxaborol-2-yl)pyrrolidin-1-yl)propan-1-one hydrochloride according to SP-5. The tBu group was removed with 4N HCl in 1,4-dioxane and then coupled with tert-butyl N⁶-(tert-butoxycarbonyl)-L-lysinate according to SP-5. The Boc group was removed with 4N HCl in 1,4-dioxane and was coupled with 2,2′-(7-(2-((2,5-dioxopyrrolidin-1-yl)oxy)-2-oxoethyl)-1,4,7-triazonane-1,4-diyl)diacetic acid (2.0 eq.) in anhydrous DMF and 10 eq. of TEA. All of the protecting groups were removed according to SP-6 to afford the title product (12.5 mg, 16%) as a white powder. ESI/MS (m/z): 1163 (M−H₂O+H).

### Preparation 169

### 2,2′-(4-(2-Amino-2-oxoethyl)-10-(2-(((S)-1-((2-(((S)-1-((4-(((R)-1-((2R,4S)-2-borono-4-hydroxypyrrolidin-1-yl)-1-oxopropan-2-yl)carbamoyl)quinolin-8-yl)amino)-3-carboxy-1-oxopropan-2-yl)amino)-2-oxoethyl)amino)-5-guanidino-1-oxopentan-2-yl)amino)-2-oxoethyl)-1,4,7,10-tetraazacyclododecane-1,7-diyl)diacetic acid

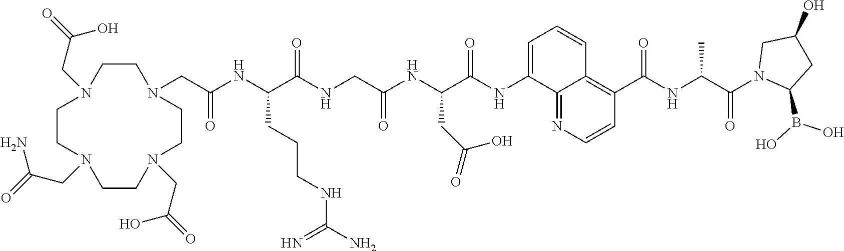 [SMILES: C[C@@H](NC(=O)c1ccnc2c(NC(=O)[C@H](CC(=O)O)NC(=O)CNC(=O)[C@H](CCCNC(=N)N)NC(=O)CN3CCN(CC(=O)O)CCN(CC(N)=O)CCN(CC(=O)O)CC3)cccc12)C(=O)N1C[C@@H](O)C[C@H]1B(O)O]

8-((5S,11S)-11-(2-(tert-Butoxy)-2-oxoethyl)-1-(9H-fluoren-9-yl)-3,6,9-trioxo-5-(3-(3-((2,2,4,6,7-pentamethyl-2,3-dihydrobenzofuran-5-yl)sulfonyl)guanidino)propyl)-2-oxa-4,7,10-triazadodecan-12-amido)quinoline-4-carboxylic acid was coupled with (2R)-2-amino-1-((2R,4S)-4-hydroxy-2-((3aS,4S,6S)-3a,5,5-trimethylhexahydro-4,6-methanobenzo[d][1,3,2]dioxaborol-2-yl)pyrrolidin-1-yl)propan-1-one hydrochloride according to SP-5. The Fmoc protecting group was removed with piperidine (20%) in DMF and was coupled with 2-(7-(2-amino-2-oxoethyl)-4,10-bis(2-(tert-butoxy)-2-oxoethyl)-1,4,7,10-tetraazacyclododecan-1-yl)acetic acid according to SP-5. All the protecting groups were removed according to SP-6 to afford the title compound (4.4 mg, 15%). ESI/MS (m/z): 1069 (M−H₂O+H).

### Preparation 170

### (2S,8S,18S,27R)-2-((4-(((R)-1-((R)-2-Boronopyrrolidin-1-yl)-1-oxopropan-2-yl)carbamoyl)benzyl)carbamoyl)-8-(3-guanidinopropyl)-4,7,10,16,24-pentaoxo-27-(4,7,10-tris(carboxymethyl)-1,4,7,10-tetraazacyclododecan-1-yl)-3,6,9,17,23-pentaazaheptacosane-1,18,27-tricarboxylic acid

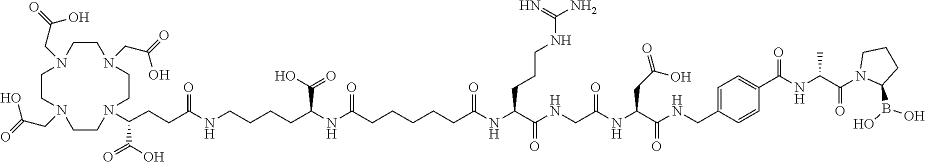 [SMILES: C[C@@H](NC(=O)c1ccc(CNC(=O)[C@H](CC(=O)O)NC(=O)CNC(=O)[C@H](CCCNC(=N)N)NC(=O)CCCCCC(=O)N[C@@H](CCCCNC(=O)CC[C@H](C(=O)O)N2CCN(CC(=O)O)CCN(CC(=O)O)CCN(CC(=O)O)CC2)C(=O)O)cc1)C(=O)N1CCC[C@H]1B(O)O]

tert-Butyl (3S)-3-(2-((S)-2-amino-5-(3-((2,2,4,6,7-pentamethyl-2,3-dihydrobenzofuran-5-yl)sulfonyl)guanidino)pentanamido)acetamido)-4-oxo-4-((4-(((2R)-1-oxo-1-((2R)-2-((3aS,4S,6S)-3a,5,5-trimethylhexahydro-4,6-methanobenzo[d][1,3,2]dioxaborol-2-yl)pyrrolidin-1-yl)propan-2-yl)carbamoyl)benzyl)amino)butanoate was coupled with (S)-7-((6-((((9H-fluoren-9-yl)methoxy)carbonyl)amino)-1-(tert-butoxy)-1-oxohexan-2-yl)amino)-7-oxoheptanoic acid according to SP-5. The Fmoc protecting group was removed with piperidine (5%) in DMF and was conjugated with (R)-2,2′,2″-(10-(1-carboxy-4-(4-nitrophenoxy)-4-oxobutyl)-1,4,7,10-tetraazacyclododecane-1,4,7-triyl)triacetic acid (2.0 eq.) in anhydrous DMF and 10 eq. of TEA. All the protecting groups were removed according to SP-6 to afford the title compound (9.7 mg, 28%) as a white powder. ESI/MS (m/z): 1359 (M−H₂O+H).

The following compound in Table 20 was synthesized in essentially the same manner as described for (2S,8S,18S,27R)-2-((4-(((R)-1-((R)-2-boronopyrrolidin-1-yl)-1-oxopropan-2-yl)carbamoyl)benzyl)carbamoyl)-8-(3-guanidinopropyl)-4,7,10,16,24-pentaoxo-27-(4,7,10-tris(carboxymethyl)-1,4,7,10-tetraazacyclododecan-1-yl)-3,6,9,17,23-pentaazaheptacosane-1,18,27-tricarboxylic acid.

TABLE 20

| | | ESI/MS |
|---|---|---|
| Prep # | Structure | m/z |
| 171 | 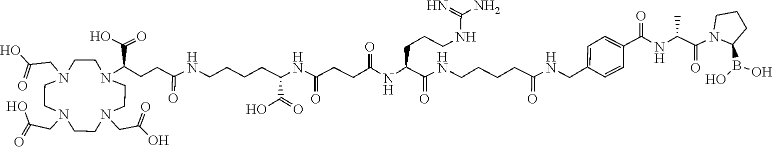 [SMILES: C[C@@H](NC(=O)c1ccc(CNC(=O)CCCCNC(=O)[C@H](CCCNC(=N)N)NC(=O)CCC(=O)N[C@@H](CCCCNC(=O)CC[C@H](C(=O)O)N2CCN(CC(=O)O)CCN(CC(=O)O)CCN(CC(=O)O)CC2)C(=O)O)cc1)C(=O)N1CCC[C@H]1B(O)O] | 1244 (M − H₂O + H) |
| | 2,2′,2″-(10-((10S,17S,26R)-1-(4-(((R)-1-((R)-2-Boronopyrrolidin-1-yl)-1- | |
| | oxopropan-2-yl)carbamoyl)phenyl)-17,26-dicarboxy-10-(3-guanidinopropyl)- | |
| | 3,9,12,15,23-pentaoxo-2,8,11,16,22-pentaazahexacosan-26-yl)-1,4,7,10- | |
| | tetraazacyclododecane-1,4,7-triyl)triacetic acid | |

### Preparation 172

### 2,2′-(7-((10S,20S)-1-(4-(((R)-1-((R)-2-Boronopyrrolidin-1-yl)-1-oxopropan-2-yl)carbamoyl)phenyl)-20-carboxy-10-(3-(3,3-dimethylguanidino)propyl)-3,9,12,18,26-pentaoxo-2,8,11,19,25-pentaazaheptacosan-27-yl)-1,4,7-triazonane-1,4-diyl)diacetic acid

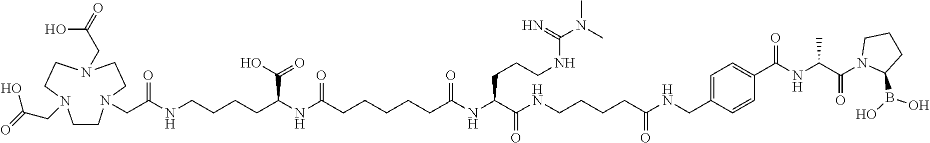 [SMILES: C[C@@H](NC(=O)c1ccc(CNC(=O)CCCCNC(=O)[C@H](CCCNC(=N)N(C)C)NC(=O)CCCCCC(=O)N[C@@H](CCCCNC(=O)CN2CCN(CC(=O)O)CCN(CC(=O)O)CC2)C(=O)O)cc1)C(=O)N1CCC[C@H]1B(O)O]

The tBu group of tert-butyl 7-(((8S)-3-imino-2-methyl-9,15-dioxo-17-(4-(((2R)-1-oxo-1-((2R)-2-((3aS,4S,6S)-3a,5,5-trimethylhexahydro-4,6-methanobenzo[d][1,3,2]dioxaborol-2-yl)pyrrolidin-1-yl)propan-2-yl)carbamoyl)phenyl)-2,4,10,16-tetraazaheptadecan-8-yl)amino)-7-oxoheptanoate was removed with 4N HCl in 1,4-dioxane and was coupled with tert-butyl N⁶-(tert-butoxycarbonyl)-L-lysinate according to SP-5. Both Boc and -tBu groups were removed with 30% TFA in DCM (with the addition of 3% of TIPS) and then coupled with 2,2′-(7-(2-((2,5-dioxopyrrolidin-1-yl)oxy)-2-oxoethyl)-1,4,7-triazonane-1,4-diyl)diacetic acid (2.0 eq.) in anhydrous DMF and 10 eq. of TEA. The pinanediol protecting group was removed according to SP-6 to afford the title product (23.8 mg, 32%) as a white powder. ESI/MS (m/z): 1141 (M−H₂O+H).

### Preparation 173

### 2,2′-(7-((10S,20S,29R)-1-(4-(((R)-1-((R)-2-Boronopyrrolidin-1-yl)-1-oxopropan-2-yl)carbamoyl)phenyl)-20,29-dicarboxy-10-(3-(3,3-dimethylguanidino)propyl)-3,9,12,18,26-pentaoxo-2,8,11,19,25-pentaazanonacosan-29-yl)-1,4,7-triazonane-1,4-diyl)diacetic acid

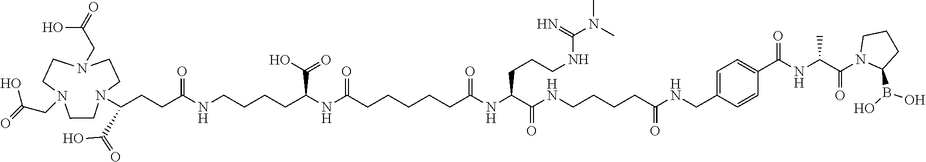 [SMILES: C[C@@H](NC(=O)c1ccc(CNC(=O)CCCCNC(=O)[C@H](CCCNC(=N)N(C)C)NC(=O)CCCCCC(=O)N[C@@H](CCCCNC(=O)CC[C@H](C(=O)O)N2CCN(CC(=O)O)CCN(CC(=O)O)CC2)C(=O)O)cc1)C(=O)N1CCC[C@H]1B(O)O]

The tBu group of tert-butyl 7-(((8S)-3-imino-2-methyl-9,15-dioxo-17-(4-(((2R)-1-oxo-1-((2R)-2-((3aS,4S,6S)-3a,5,5-trimethylhexahydro-4,6-methanobenzo[d][1,3,2]dioxaborol-2-yl)pyrrolidin-1-yl)propan-2-yl)carbamoyl)phenyl)-2,4,10,16-tetraazaheptadecan-8-yl)amino)-7-oxoheptanoate was removed with 4N HCl in 1,4-dioxane and then coupled with tert-butyl N⁶-(((9H-fluoren-9-yl)methoxy)carbonyl)-L-lysinate according to SP-5. The Fmoc protecting group was removed with piperidine (5%) in DMF and was coupled to (R)-4-(4,7-bis(2-(tert-butoxy)-2-oxoethyl)-1,4,7-triazonan-1-yl)-5-(tert-butoxy)-5-oxopentanoic acid according to SP-5 using PyBrop instead of HATU. All the protecting groups were removed according to SP-6 to afford the title compound (10.3 mg, 13%) as a white powder. ESI/MS (m/z): 1213 (M−H₂O+H).

The following compounds in Table 21 were synthesized in essentially the same manner as described for 2,2′-(7-((10S,20S,29R)-1-(4-(((R)-1-((R)-2-boronopyrrolidin-1-yl)-1-oxopropan-2-yl)carbamoyl)phenyl)-20,29-dicarboxy-10-(3-(3,3-dimethylguanidino)propyl)-3,9,12,18,26-pentaoxo-2,8,11,19,25-pentaazanonacosan-29-yl)-1,4,7-triazonane-1,4-diyl)diacetic acid.

TABLE 21

| | | ESI/MS |
|---|---|---|
| Prep # | Structure | m/z |
| 174 | 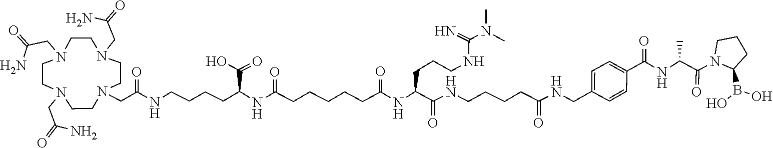 [SMILES: C[C@@H](NC(=O)c1ccc(CNC(=O)CCCCNC(=O)[C@H](CCCNC(=N)N(C)C)NC(=O)CCCCCC(=O)N[C@@H](CCCCNC(=O)CN2CCN(CC(N)=O)CCN(CC(N)=O)CCN(CC(N)=O)CC2)C(=O)O)cc1)C(=O)N1CCC[C@H]1B(O)O] | 1240 (M − H₂O + H) |
| | N2-(7-(((S)-17-(4-(((R)-1-((R)-2-Boronopyrrolidin-1-yl)-1-oxopropan-2- | |
| | yl)carbamoyl)phenyl)-3-imino-2-methyl-9,15-dioxo-2,4,10,16- | |
| | tetraazaheptadecan-8-yl)amino)-7-oxoheptanoyl)-N6-(2-(4,7,10-tris(2-amino-2- | |
| | oxoethyl)-1,4,7,10-tetraazacyclododecan-1-yl)acetyl)-L-lysine | |
| 175 | 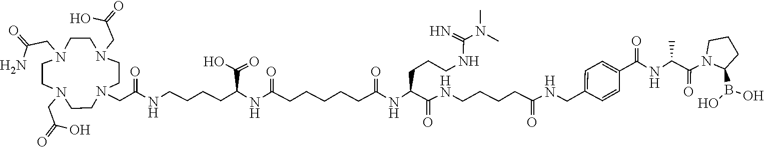 [SMILES: C[C@@H](NC(=O)c1ccc(CNC(=O)CCCCNC(=O)[C@H](CCCNC(=N)N(C)C)NC(=O)CCCCCC(=O)N[C@@H](CCCCNC(=O)CN2CCN(CC(=O)O)CCN(CC(N)=O)CCN(CC(=O)O)CC2)C(=O)O)cc1)C(=O)N1CCC[C@H]1B(O)O] | 1241 (M − H₂O + H) |
| | 2,2′-(4-(2-Amino-2-oxoethyl)-10-((10S,20S)-1-(4-(((R)-1-((R)-2- | |
| | boronopyrrolidin-1-yl)-1-oxopropan-2-yl)carbamoyl)phenyl)-20-carboxy-10-(3- | |
| | (3,3-dimethylguanidino)propyl)-3,9,12,18,26-pentaoxo-2,8,11,19,25- | |
| | pentaazaheptacosan-27-yl)-1,4,7,10-tetraazacyclododecane-1,7-diyl)diacetic acid | |

### Preparation 176

### 2,2′,2″-(10-((10S,20S)-1-(4-(((R)-1-((R)-2-Boronopyrrolidin-1-yl)-1-oxopropan-2-yl)carbamoyl)phenyl)-20-carboxy-10-(3-(3,3-dimethylguanidino)propyl)-3,9,12,18,26-pentaoxo-2,8,11,19,25-pentaazaheptacosan-27-yl)-1,4,7,10-tetraazacyclododecane-1,4,7-triyl)triacetic acid

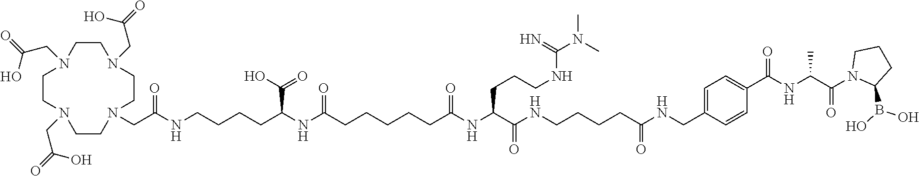 [SMILES: C[C@@H](NC(=O)c1ccc(CNC(=O)CCCCNC(=O)[C@H](CCCNC(=N)N(C)C)NC(=O)CCCCCC(=O)N[C@@H](CCCCNC(=O)CN2CCN(CC(=O)O)CCN(CC(=O)O)CCN(CC(=O)O)CC2)C(=O)O)cc1)C(=O)N1CCC[C@H]1B(O)O]

The tBu group of tert-butyl 7-(((8S)-3-imino-2-methyl-9,15-dioxo-17-(4-(((2R)-1-oxo-1-((2R)-2-((3aS,4S,6S)-3a,5,5-trimethylhexahydro-4,6-methanobenzo[d][1,3,2]dioxaborol-2-yl)pyrrolidin-1-yl)propan-2-yl)carbamoyl)phenyl)-2,4,10,16-tetraazaheptadecan-8-yl)amino)-7-oxoheptanoate was removed with 4N HCl in 1,4-dioxane and was coupled to preloaded N⁶-(tert-butoxycarbonyl)-L-lysine on resin according to SP-2 using PyBrop instead of HATU. The peptide was cleaved from the resin according to SP-4 (4N HCl in 1,4-dioxane was added to ensure the Boc protection at Lys was completely removed) and was coupled with 2,2′,2″-(10-(2-((2,5-dioxopyrrolidin-1-yl)oxy)-2-oxoethyl)-1,4,7,10-tetraazacyclododecane-1,4,7-triyl)triacetic acid (2.0 eq.) in anhydrous DMF and 10 eq. of DIPEA. The pinanediol protecting group was removed according to SP-6 to afford the title compound (19 mg, 23%) as a white powder. ESI/MS (m/z): 1242 (M−H₂O+H).

### Preparation 177

### 2,2′-(4-(2-Amino-2-oxoethyl)-10-((10S,20S)-1-(4-(((R)-1-((R)-2-boronopyrrolidin-1-yl)-1-oxopropan-2-yl)carbamoyl)phenyl)-20-carboxy-10-(3-guanidinopropyl)-3,9,12,18,26-pentaoxo-2,8,11,19,25-pentaazaheptacosan-27-yl)-1,4,7,10-tetraazacyclododecane-1,7-diyl)diacetic acid

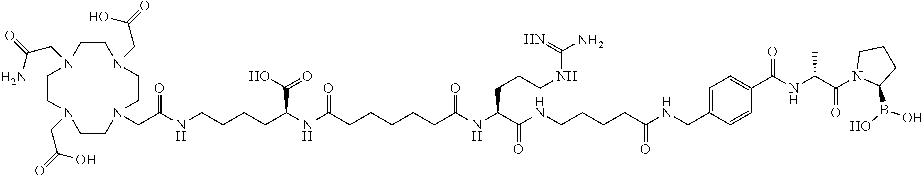 [SMILES: C[C@@H](NC(=O)c1ccc(CNC(=O)CCCCNC(=O)[C@H](CCCNC(=N)N)NC(=O)CCCCCC(=O)N[C@@H](CCCCNC(=O)CN2CCN(CC(=O)O)CCN(CC(N)=O)CCN(CC(=O)O)CC2)C(=O)O)cc1)C(=O)N1CCC[C@H]1B(O)O]

(9R,19S)-19-(3-((E)-2,3-Bis(tert-butoxycarbonyl)guanidino)propyl)-9-(tert-butoxycarbonyl)-1-(9H-fluoren-9-yl)-3,11,17,20-tetraoxo-2-oxa-4,10,18,21-tetraazahexacosan-26-oic acid was coupled with 4-(aminomethyl)-N-((2R)-1-oxo-1-((2R)-2-((3aS,4S,6S)-3a,5,5-trimethylhexahydro-4,6-methanobenzo[d][1,3,2]dioxaborol-2-yl)pyrrolidin-1-yl)propan-2-yl)benzamide hydrochloride according to SP-5. The Fmoc protecting group was removed with piperidine (5%) in DMF and was coupled with 2-(7-(2-amino-2-oxoethyl)-4,10-bis(2-(tert-butoxy)-2-oxoethyl)-1,4,7,10-tetraazacyclododecan-1-yl)acetic acid according to SP-5. All the protecting groups were removed according to SP-6 to afford the title product (8 mg, 25%) as a white powder. ESI/MS (m/z): 1213 (M−H₂O+H).

### Preparation 178

### 2,2′-(4-(2-Amino-2-oxoethyl)-10-((10S,20S)-1-(4-(((R)-1-((R)-2-boronopyrrolidin-1-yl)-1-oxopropan-2-yl)carbamoyl)phenyl)-20-carboxy-10-(3-guanidinopropyl)-3,9,12,15,18,26-hexaoxo-2,8,11,16,19,25-hexaazaheptacosan-27-yl)-1,4,7,10-tetraazacyclododecane-1,7-diyl)diacetic acid

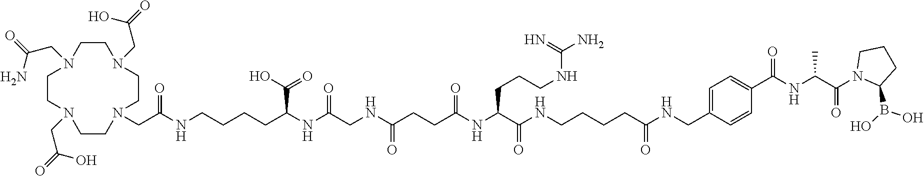 [SMILES: C[C@@H](NC(=O)c1ccc(CNC(=O)CCCCNC(=O)[C@H](CCCNC(=N)N)NC(=O)CCC(=O)NCC(=O)N[C@@H](CCCCNC(=O)CN2CCN(CC(=O)O)CCN(CC(N)=O)CCN(CC(=O)O)CC2)C(=O)O)cc1)C(=O)N1CCC[C@H]1B(O)O]

4-Oxo-4-(((2S)-1-oxo-1-((5-oxo-5-((4-(((2R)-1-oxo-1-((2R)-2-((3aS,4S,6S)-3a,5,5-trimethylhexahydro-4,6-methanobenzo[d][1,3,2]dioxaborol-2-yl)pyrrolidin-1-yl)propan-2-yl)carbamoyl)benzyl)amino)pentyl)amino)-5-(3-((2,2,4,5,7-pentamethyl-2,3-dihydrobenzofuran-6-yl)sulfonyl)guanidino)pentan-2-yl)amino)butanoic acid was coupled with tert-butyl glycinate according to SP-5. The tBu group was removed with 4N HCl in 1,4-dioxane and was coupled with tert-butyl N⁶-(tert-butoxycarbonyl)-L-lysinate according to SP-5. The Boc protecting group on the Lys moiety was selectively removed with 1N HCl in a mixture of 1,4-dioxane and DCM (1:3) to give the free amine which was coupled with 2-(7-(2-amino-2-oxoethyl)-4,10-bis(2-(tert-butoxy)-2-oxoethyl)-1,4,7,10-tetraazacyclododecan-1-yl)acetic acid according to SP-5. All the protecting groups were removed according to SP-6 to afford the title compound (10.2 mg, 32%) as a white powder. ESI/MS (m/z): 1228 (M−H₂O+H).

### Preparation 179

### 2,2′,2″-(10-((2S,8S,11S)-1-((4-(((R)-1-((2R,4S)-2-Borono-4-hydroxypyrrolidin-1-yl)-1-oxopropan-2-yl)carbamoyl)quinolin-8-yl)amino)-2-(carboxymethyl)-8-(3-guanidinopropyl)-11-(4-(4-iodophenyl)butanamido)-1,4,7,10,17-pentaoxo-3,6,9,16-tetraazaoctadecan-18-yl)-1,4,7,10-tetraazacyclododecane-1,4,7-triyl)triacetic acid

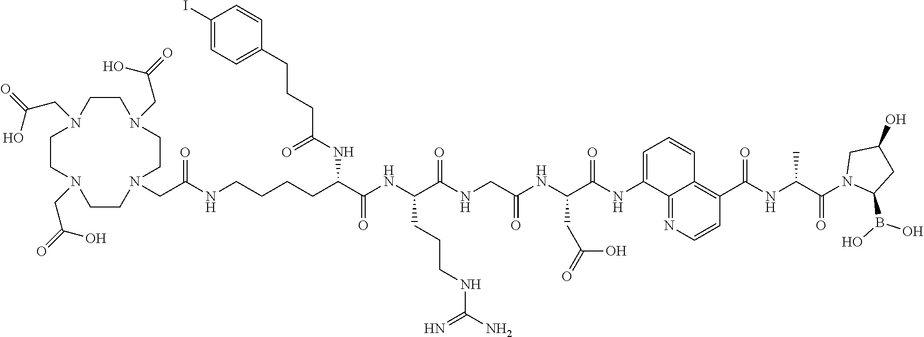 [SMILES: C[C@@H](NC(=O)c1ccnc2c(NC(=O)[C@H](CC(=O)O)NC(=O)CNC(=O)[C@H](CCCNC(=N)N)NC(=O)[C@H](CCCCNC(=O)CN3CCN(CC(=O)O)CCN(CC(=O)O)CCN(CC(=O)O)CC3)NC(=O)CCCc3ccc(I)cc3)cccc12)C(=O)N1C[C@@H](O)C[C@H]1B(O)O]

8-((5S,11S)-11-(2-(tert-Butoxy)-2-oxoethyl)-1-(9H-fluoren-9-yl)-3,6,9-trioxo-5-(3-(3-((2,2,4,6,7-pentamethyl-2,3-dihydrobenzofuran-5-yl)sulfonyl)guanidino)propyl)-2-oxa-4,7,10-triazadodecan-12-amido)quinoline-4-carboxylic acid was coupled with (2R)-2-amino-1-((2R,4S)-4-hydroxy-2-((3aS,4S,6S)-3a,5,5-trimethylhexahydro-4,6-methanobenzo[d][1,3,2]dioxaborol-2-yl)pyrrolidin-1-yl)propan-1-one hydrochloride according to SP-5. The Fmoc protecting group was removed with piperidine (20%) in DMF and was coupled with N⁶-(((9H-fluoren-9-yl)methoxy)carbonyl)-N²-(tert-butoxycarbonyl)-L-lysine according to SP-5. The Fmoc protecting group was removed with piperidine (20%) in DMF and was coupled to 4-(p-iodophenyl)butyric acid according to SP-5. The Boc protecting group was removed with 4N HCl in 1,4-dioxane and was coupled with 2,2′,2″-(10-(2-((2,5-dioxopyrrolidin-1-yl)oxy)-2-oxoethyl)-1,4,7,10-tetraazacyclododecane-1,4,7-triyl)triacetic acid (2.0 eq.) in anhydrous DMF and 10 eq. of DIPEA. All the protecting groups were removed according to SP-6 to afford the title compound (7 mg, 8%) as a white powder. ESI/MS (m/z): 727 (M/2-H₂O+H).

### Preparation 180

### 2,2′-(4-(2-Amino-2-oxoethyl)-10-((10S,20S)-1-(4-(((R)-1-((R)-2-boronopyrrolidin-1-yl)-1-oxopropan-2-yl)carbamoyl)naphthalen-1-yl)-20-carboxy-10-(3-guanidinopropyl)-1,9,12,18,26-pentaoxo-2,8,11,19,25-pentaazaheptacosan-27-yl)-1,4,7,10-tetraazacyclododecane-1,7-diyl)diacetic acid

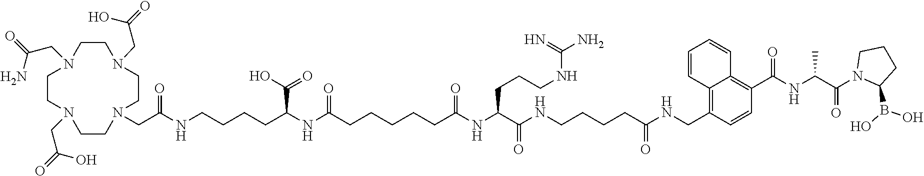 [SMILES: C[C@@H](NC(=O)c1ccc(CNC(=O)CCCCNC(=O)[C@H](CCCNC(=N)N)NC(=O)CCCCCC(=O)N[C@@H](CCCCNC(=O)CN2CCN(CC(=O)O)CCN(CC(N)=O)CCN(CC(=O)O)CC2)C(=O)O)c2ccccc12)C(=O)N1CCC[C@H]1B(O)O]

The Boc protecting group of tert-butyl N⁶-(tert-butoxycarbonyl)-N²-(7-oxo-7-(((2S)-1-oxo-1-((5-(4-(((2R)-1-oxo-1-((2R)-2-((3aS,4S,6S)-3a,5,5-trimethylhexahydro-4,6-methanobenzo[d][1,3,2]dioxaborol-2-yl)pyrrolidin-1-yl)propan-2-yl)carbamoyl)-1-naphthamido)pentyl)amino)-5-(3-((2,2,4,6,7-pentamethyl-2,3-dihydrobenzofuran-5-yl)sulfonyl)guanidino)pentan-2-yl)amino)heptanoyl)-L-lysinate was selectively removed with 1N HCl in a mixture of 1,4-dioxane and DCM (1:3) to give the free amine which was coupled with 2-(7-(2-amino-2-oxoethyl)-4,10-bis(2-(tert-butoxy)-2-oxoethyl)-1,4,7,10-tetraazacyclododecan-1-yl)acetic acid according to SP-5. All the protecting groups were removed according to SP-6 to afford the title compound (9 mg, 28%) as a white powder. ESI/MS (m/z): 1263 (M−H₂O+H).

### Preparation 181

### 2,2′,2″-(10-((6S,16S,25R)-1-Amino-6-(4-(3-((4-(((R)-1-((R)-2-boronopyrrolidin-1-yl)-1-oxopropan-2-yl)carbamoyl)benzyl)amino)-3-oxopropyl)piperazine-1-carbonyl)-16,25-dicarboxy-1-imino-8,11,14,22-tetraoxo-2,7,12,15,21-pentaazapentacosan-25-yl)-1,4,7,10-tetracyclodecane-1,4,7-triacetic acid

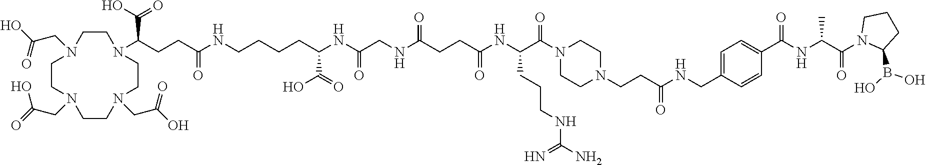

4-[1,1-Dimethylethoxy)carbonyl]-1-piperazinepropanoic acid was coupled with (2R)-2-amino-1-((2R)-2-((3aS,4S,6S)-3a,5,5-trimethylhexahydro-4,6-methanobenzo[d][1,3,2]dioxaborol-2-yl)pyrrolidin-1-yl)propan-1-one hydrochloride according to SP-5. The Boc protecting group was then removed with 4N HCl in 1,4-dioxane and was coupled to N²-(4-((2-(tert-butoxy)-2-oxoethyl)amino)-4-oxobutanoyl)-N^{w}-((2,2,4,5,7-pentamethyl-2,3-dihydrobenzofuran-6-yl)sulfonyl)-L-arginine according to SP-5. The tBu protecting group was removed with 4N HCl in 1,4-dioxane and was coupled with tert-butyl N⁶-(tert-butoxycarbonyl)-L-lysinate according to SP-5. The Boc protecting group was removed with 4N HCl in 1,4-dioxane and was coupled with 2,2′,2″-(10-(2-(4-nitrophenoxy)-2-oxoethyl)-1,4,7,10-tetraazacyclododecane-1,4,7-triyl)triacetic acid (2.0 eq.) in anhydrous DMF and 10 eq. of DIPEA. All the protecting groups were removed according to SP-6 to afford the title product (17.3 mg, 10%) as a white powder. ESI/MS (m/z): 1342 (M−H₂O+H).

### Preparation 182

### 2,2′-(4-(2-Amino-2-oxoethyl)-10-(2-(((S)-1-((5-((4-(((R)-1-((2R,4S)-2-borono-4-hydroxypyrrolidin-1-yl)-1-oxopropan-2-yl)carbamoyl)benzyl)amino)-5-oxopentyl)amino)-5-guanidino-1-oxopentan-2-yl)amino)-2-oxoethyl)-1,4,7,10-tetraazacyclododecane-1,7-diyl)diacetic acid

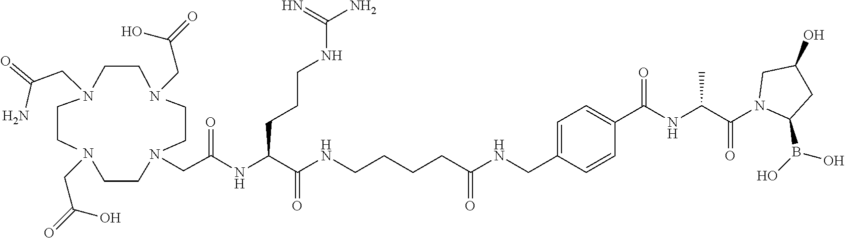

The initial resin loading with 4-[(N-Fmoc-amino)methyl]benzoic acid was performed as described in SP-1. The Fmoc protecting group was removed according to SP-3 and was coupled with Fmoc-5-aminovaleric acid according to SP-2 using PyBrop as the coupling reagent. The Fmoc protecting group was removed according to SP-3 and was coupled with N²-(((9H-fluoren-9-yl)methoxy)carbonyl)-N^{w}-((2,2,4,6,7-pentamethyl-2,3-dihydrobenzofuran-5-yl)sulfonyl)-L-arginine according to SP-2. The Fmoc protecting group was removed according to SP-3 and was coupled with 2-(7-(2-amino-2-oxoethyl)-4,10-bis(2-(tert-butoxy)-2-oxoethyl)-1,4,7,10-tetraazacyclododecan-1-yl)acetic acid according to SP-2 using PyBrop as the coupling reagent. The peptide was cleaved from the resin according to SP-4 and was coupled with (2R)-2-amino-1-((2R,4S)-4-hydroxy-2-((3aS,4S,6S)-3a,5,5-trimethylhexahydro-4,6-methanobenzo[d][1,3,2]dioxaborol-2-yl)pyrrolidin-1-yl)propan-1-one hydrochloride according to SP-5. All the protecting groups were removed according to SP-6 to afford the title compound (17.7 mg, 13%). ESI/MS (m/z): 959 (M−H₂O+H).

### Preparation 183

### 2,2′,2″-(10-((4S,10S)-1-(4-(((R)-1-((2R,4S)-2-Borono-4-hydroxypyrrolidin-1-yl)-1-oxopropan-2-yl)carbamoyl)phenyl)-4-(carboxymethyl)-10-(3-guanidinopropyl)-3,6,9,12-tetraoxo-2,5,8,11-tetraazatridecan-13-yl)-1,4,7,10-tetraazacyclododecane-1,4,7-triyl)triacetic acid

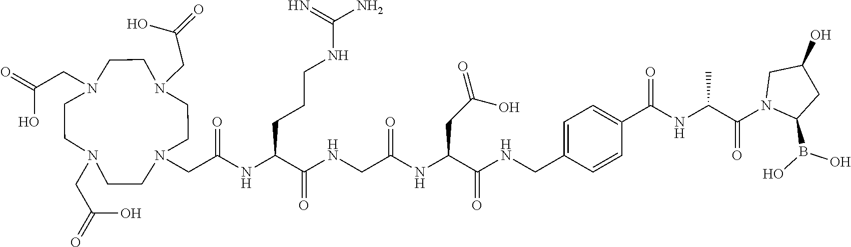 [SMILES: C[C@@H](NC(=O)c1ccc(CNC(=O)[C@H](CC(=O)O)NC(=O)CNC(=O)[C@H](CCCNC(=N)N)NC(=O)CN2CCN(CC(=O)O)CCN(CC(=O)O)CCN(CC(=O)O)CC2)cc1)C(=O)N1C[C@@H](O)C[C@H]1B(O)O]

The initial resin loading with 4-(((((9H-fluoren-9-yl)methoxy)carbonyl)amino)methyl)benzoic acid was performed according to SP-1. The Fmoc protecting group was removed according to SP-3 and was coupled with (5S,11S)-11-(2-(tert-butoxy)-2-oxoethyl)-1-(9H-fluoren-9-yl)-3,6,9-trioxo-5-(3-(3-((2,2,4,6,7-pentamethyl-2,3-dihydrobenzofuran-5-yl)sulfonyl)guanidino)propyl)-2-oxa-4,7,10-triazadodecan-12-oic acid according to SP-2. The Fmoc protecting group was removed according to SP-3 and was coupled with 2-(7-(2-amino-2-oxoethyl)-4,10-bis(2-(tert-butoxy)-2-oxoethyl)-1,4,7,10-tetraazacyclododecan-1-yl)acetic acid according to SP-2 except using PyBrop as the coupling reagent. The peptide was cleaved from the resin according to SP-4 and was coupled with (2R)-2-amino-1-((2R,4S)-4-hydroxy-2-((3aS,4S,6S)-3a,5,5-trimethylhexahydro-4,6-methanobenzo[d][1,3,2]dioxaborol-2-yl)pyrrolidin-1-yl)propan-1-one hydrochloride according to SP-5. All the protecting groups were removed according to SP-6 to afford the title product (22 mg, 31%). ESI/MS (m/z): 1033 (M−H₂O+H).

### Preparation 184

### 2,2′,2″-(10-((10S,20S,29R)-1-(4-(((R)-1-((R)-2-Boronopyrrolidin-1-yl)-1-oxopropan-2-yl)carbamoyl)phenyl)-20,29-dicarboxy-10-(3-(3,3-dimethylguanidino)propyl)-3,9,12,15,18,26-hexaoxo-2,8,11,16,19,25-hexaazanonacosan-29-yl)-1,4,7,10-tetraazacyclododecane-1,4,7-triyl)triacetic acid

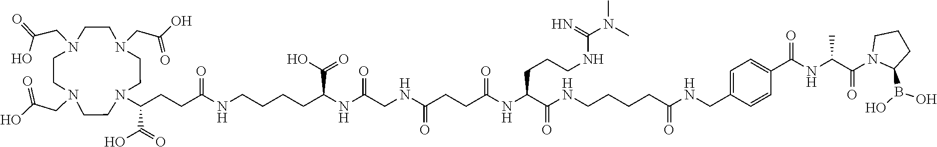 [SMILES: C[C@@H](NC(=O)c1ccc(CNC(=O)CCCCNC(=O)[C@H](CCCNC(=N)N(C)C)NC(=O)CCC(=O)NCC(=O)N[C@@H](CCCCNC(=O)CC[C@H](C(=O)O)N2CCN(CC(=O)O)CCN(CC(=O)O)CCN(CC(=O)O)CC2)C(=O)O)cc1)C(=O)N1CCC[C@H]1B(O)O]

The initial resin loading with N²-(((9H-fluoren-9-yl)methoxy)carbonyl)-N⁶-(1-(4,4-dimethyl-2,6-dioxocyclohexylidene)ethyl)-L-lysine was performed according to SP-1. The Fmoc protecting group was removed according to SP-3 and was coupled with (((9H-fluoren-9-yl)methoxy)carbonyl)glycine according to SP-2. The Dde protecting group was removed with imidazole/NH₂OH hydrochloride in DMF and DCM with the addition of NMP and was coupled with (R)-5-(tert-butoxy)-5-oxo-4-(4,7,10-tris(2-(tert-butoxy)-2-oxoethyl)-1,4,7,10-tetraazacyclododecan-1-yl)pentanoic acid according to SP-2. The Fmoc protecting group was removed according to SP-3 and was reacted with succinic anhydride in DMF and DIPEA. The resulting product was coupled with 4-((S,Z)-8-amino-2-methyl-9,15-dioxo-3-((2,2,4,6,7-pentamethyl-2,3-dihydrobenzofuran)-5-sulfonamido)-2,4,10,16-tetraazaheptadec-3-en-17-yl)-N-((2R)-1-oxo-1-((2R)-2-((3aS,4S,6S)-3a,5,5-trimethylhexahydro-4,6-methanobenzo[d][1,3,2]dioxaborol-2-yl)pyrrolidin-1-yl)propan-2-yl)benzamide according to SP-2 except using PyBrop as the coupling reagent. The peptide was cleaved from the resin and the protecting groups removed according to SP-6 to afford the title compound (5.8 mg, 7%). ESI/MS (m/z): 1029 (M−H₂O+H).

### Preparation 185

### 2,2′,2″-(10-(2-(((S)-17-(4-(((R)-1-((R)-2-Boronopyrrolidin-1-yl)-1-oxopropan-2-yl)carbamoyl)phenyl)-3-imino-2-methyl-9,15-dioxo-2,4,10,16-tetraazaheptadecan-8-yl)amino)-2-oxoethyl)-1,4,7,10-tetraazacyclododecane-1,4,7-triyl)triacetic acid

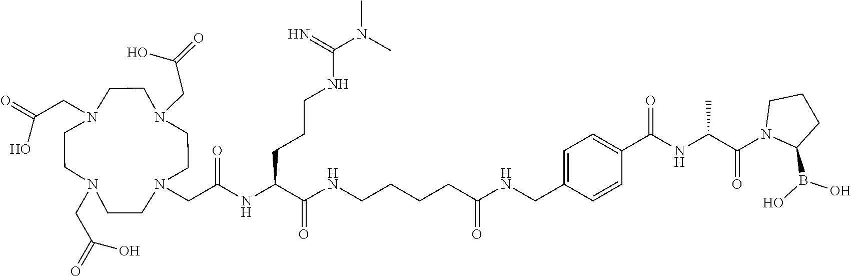 [SMILES: C[C@@H](NC(=O)c1ccc(CNC(=O)CCCCNC(=O)[C@H](CCCNC(=N)N(C)C)NC(=O)CN2CCN(CC(=O)O)CCN(CC(=O)O)CCN(CC(=O)O)CC2)cc1)C(=O)N1CCC[C@H]1B(O)O]

The pinanediol protecting group of 4-((S)-8-amino-3-imino-2-methyl-9,15-dioxo-2,4,10,16-tetraazaheptadecan-17-yl)-N-((2R)-1-oxo-1-((2R)-2-((3aS,4S,6S)-3a,5,5-trimethylhexahydro-4,6-methanobenzo[d][1,3,2]dioxaborol-2-yl)pyrrolidin-1-yl)propan-2-yl)benzamide) was removed with PhB(OH)₂ in 0.01N HCl and MTBE and was coupled with 2,2′,2″-(10-(2-((2,5-dioxopyrrolidin-1-yl)oxy)-2-oxoethyl)-1,4,7,10-tetraazacyclododecane-1,4,7-triyl)triacetic acid (2.0 eq.) in anhydrous DMF and 10 eq. of DIPEA to afford the title compound (53.9 mg, 41%). ESI/MS (m/z): 972 (M−H₂O+H).

### Preparation 186

### 2,2′,2″-(10-(2-(((S)-1-((5-((((1R,4S)-4-(((R)-1-((R)-2-Boronopyrrolidin-1-yl)-1-oxopropan-2-yl)carbamoyl)cyclohexyl)methyl)amino)-5-oxopentyl)amino)-5-guanidino-1-oxopentan-2-yl)amino)-2-oxoethyl)-1,4,7,10-tetraazacyclododecane-1,4,7-triyl)triacetic acid

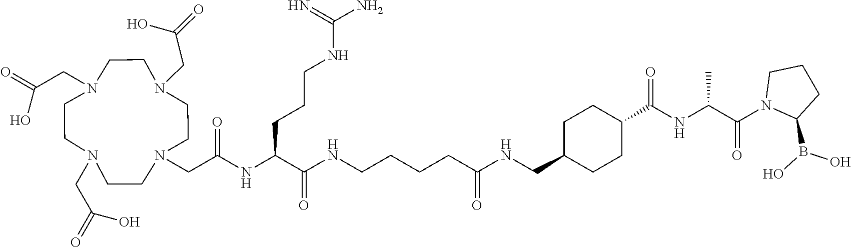 [SMILES: C[C@@H](NC(=O)[C@H]1CC[C@H](CNC(=O)CCCCNC(=O)[C@H](CCCNC(=N)N)NC(=O)CN2CCN(CC(=O)O)CCN(CC(=O)O)CCN(CC(=O)O)CC2)CC1)C(=O)N1CCC[C@H]1B(O)O]

The initial resin loading with (1r,4r)-4-(((((9H-fluoren-9-yl)methoxy)carbonyl)amino)methyl)cyclohexane-1-carboxylic acid was performed according to SP-1. The Fmoc protecting group was removed according to SP-3 except using DBU (2%) instead of piperidine in DMF and was coupled with 5-((((9H-fluoren-9-yl)methoxy)carbonyl)amino)pentanoic acid according to SP-2. The Fmoc protecting group was removed according to SP-3 except using DBU (2%) instead of piperidine in DMF and was coupled with N2-(((9H-fluoren-9-yl)methoxy)carbonyl)-Nw-((2,2,4,6,7-pentamethyl-2,3-dihydrobenzofuran-5-yl)sulfonyl)-L-arginine according to SP-2. The peptide was cleaved from the resin according to SP-4 and was coupled with (2R)-2-amino-1-((2R)-2-((3aS,4S,6S)-3a,5,5-trimethylhexahydro-4,6-methanobenzo[d][1,3,2]dioxaborol-2-yl)pyrrolidin-1-yl)propan-1-one according to SP-5 with HATU plus HOAt and DIPEA in DMF. The Fmoc protecting group was removed with DBU (2%) in DMF and was coupled with 2,2′,2″-(10-(2-((2,5-dioxopyrrolidin-1-yl)oxy)-2-oxoethyl)-1,4,7,10-tetraazacyclododecane-1,4,7-triyl)triacetic acid (2.0 eq.) in anhydrous DMF and 10 eq. of DIPEA. All the protecting groups were removed according to SP-6 to afford the title compound (9.6 mg, 2%) as a white powder. ESI/MS (m/z): 949 (M−H₂O+H).

### Preparation 187

### 2,2′,2″-(10-((10S,20S)-1-(4-(((R)-1-((2R,4S)-2-Borono-4-hydroxypyrrolidin-1-yl)-1-oxopropan-2-yl)carbamoyl)naphthalen-1-yl)-20-carboxy-10-(3-(3,3-dimethylguanidino)propyl)-8-methyl-1,9,12,18,26-pentaoxo-2,8,11,19,25-pentaazaheptacosan-27-yl)-1,4,7,10-tetraazacyclododecane-1,4,7-triyl)triacetic acid

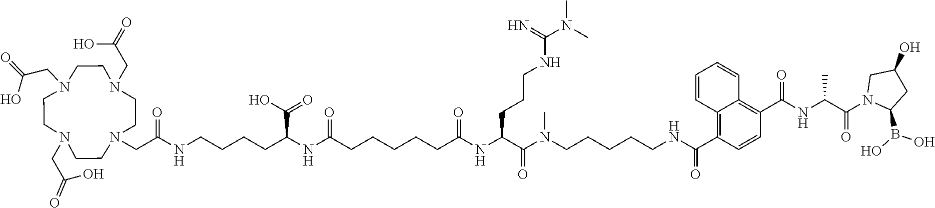 [SMILES: C[C@@H](NC(=O)c1ccc(C(=O)NCCCCCN(C)C(=O)[C@H](CCCNC(=N)N(C)C)NC(=O)CCCCCC(=O)N[C@@H](CCCCNC(=O)CN2CCN(CC(=O)O)CCN(CC(=O)O)CCN(CC(=O)O)CC2)C(=O)O)c2ccccc12)C(=O)N1C[C@@H](O)C[C@H]1B(O)O]

The initial resin loading with naphthalene-1,4-dicarboxylic acid was performed according to SP-1 and was coupled with (9H-fluoren-9-yl)methyl (S,E)-(1-((5-aminopentyl)(methyl)amino)-5-(((dimethylamino)((2,2,4,6,7-pentamethyl-2,3-dihydrobenzofuran)-5-sulfonamido)methylene)amino)-1-oxopentan-2-yl)carbamate hydrochloride according to SP-2 except using PyBrop as the coupling reagent. The Fmoc protecting group was removed according to SP-3 and was coupled with 7-(4-nitrophenoxyl)-7-oxoheptanoic acid according to SP-2 without the addition of HATU, and then was coupled with tert-butyl N⁶-(tert-butoxycarbonyl)-L-lysinate according to SP-2. The Fmoc protecting group was removed according to SP-3 and was coupled with 2-(4,7,10-tris(2-(tert-butoxy)-2-oxoethyl)-1,4,7,10-tetraazacyclododecan-1-yl)acetic acid according to SP-2 except using PyBrop as the coupling reagent. The peptide was cleaved from the resin according to SP-4 and was coupled with (2R)-2-amino-1-((2R,4S)-4-hydroxy-2-((3aS,4S,6S)-3a,5,5-trimethylhexahydro-4,6-methanobenzo[d][1,3,2]dioxaborol-2-yl)pyrrolidin-1-yl)propan-1-one hydrochloride according to SP-5. All the protecting groups were removed according to SP-6 to afford the target (4 mg, 5%). ESI/MS (m/z): 1322 (M−H₂O+H).

The following compounds in Table 22 were synthesized in essentially the same manner as described for 2,2′,2″-(10-((10S,20S)-1-(4-(((R)-1-((2R,4S)-2-borono-4-hydroxypyrrolidin-1-yl)-1-oxopropan-2-yl)carbamoyl)naphthalen-1-yl)-20-carboxy-10-(3-(3,3-dimethylguanidino)propyl)-8-methyl-1,9,12,18,26-pentaoxo-2,8,11,19,25-pentaazaheptacosan-27-yl)-1,4,7,10-tetraazacyclododecane-1,4,7-triyl)triacetic acid.

TABLE 22

| Prep | | ESI/MS |
|---|---|---|
| # | Structure | m/z |
| 188 | 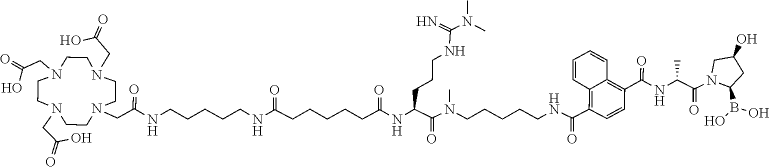 [SMILES: C[C@@H](NC(=O)c1ccc(C(=O)NCCCCCN(C)C(=O)[C@H](CCCNC(=N)N(C)C)NC(=O)CCCCCC(=O)NCCCCCNC(=O)CN2CCN(CC(=O)O)CCN(CC(=O)O)CCN(CC(=O)O)CC2)c2ccccc12)C(=O)N1C[C@@H](O)C[C@H]1B(O)O] | 1278 (M − H₂O + H) |
| | 2,2′,2″-(10-((S)-1-(4-(((R)-1-((2R,4S)-2-Borono-4-hydroxypyrrolidin-1-yl)-1- | |
| | oxopropan-2-yl)carbamoyl)naphthalen-1-yl)-10-(3-(3,3- | |
| | dimethylguanidino)propyl)-8-methyl-1,9,12,18,26-pentaoxo-2,8,11,19,25- | |
| | pentaazaheptacosan-27-yl)-1,4,7,10-tetraazacyclododecane-1,4,7-triyl)triacetic | |
| | acid | |
| 189 | 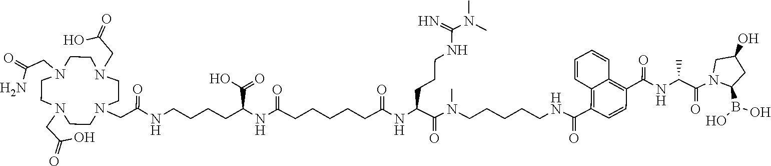 [SMILES: C[C@@H](NC(=O)c1ccc(C(=O)NCCCCCN(C)C(=O)[C@H](CCCNC(=N)N(C)C)NC(=O)CCCCCC(=O)N[C@@H](CCCCNC(=O)CN2CCN(CC(=O)O)CCN(CC(N)=O)CCN(CC(=O)O)CC2)C(=O)O)c2ccccc12)C(=O)N1C[C@@H](O)C[C@H]1B(O)O] | 1321 (M − H₂O + H) |
| | 2,2′-(4-(2-Amino-2-oxoethyl)-10-((10S,20S)-1-(4-(((R)-1-((2R,4S)-2-borono-4- | |
| | hydroxypyrrolidin-1-yl)-1-oxopropan-2-yl)carbamoyl)naphthalen-1-yl)-20- | |
| | carboxy-10-(3-(3,3-dimethylguanidino)propyl)-8-methyl-1,9,12,18,26- | |
| | pentaoxo-2,8,11,19,25-pentaazaheptacosan-27-yl)-1,4,7,10- | |
| | tetraazacyclododecane-1,7-diyl)diacetic acid | |

### Preparation 190

### 2,2′,2″-(10-(2-(((S)-1-((6-((4-(((R)-1-((2R,4S)-2-Borono-4-hydroxypyrrolidin-1-yl)-1-oxopropan-2-yl)carbamoyl)naphthalen-1-yl)amino)-6-oxohexyl)amino)-5-guanidino-1-oxopentan-2-yl)amino)-2-oxoethyl)-1,4,7,10-tetraazacyclododecane-1,4,7-triyl)triacetic acid

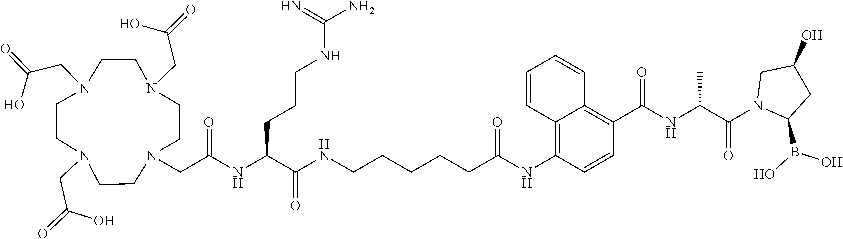 [SMILES: C[C@@H](NC(=O)c1ccc(NC(=O)CCCCCNC(=O)[C@H](CCCNC(=N)N)NC(=O)CN2CCN(CC(=O)O)CCN(CC(=O)O)CCN(CC(=O)O)CC2)c2ccccc12)C(=O)N1C[C@@H](O)C[C@H]1B(O)O]

4-(6-((((9H-Fluoren-9-yl)methoxy)carbonyl)amino)hexanamido)-1-naphthoic acid was coupled with (2R)-2-amino-1-((2R,4S)-4-hydroxy-2-((3aS,4S,6S)-3a,5,5-trimethylhexahydro-4,6-methanobenzo[d][1,3,2]dioxaborol-2-yl)pyrrolidin-1-yl)propan-1-one hydrochloride according to SP-5. The Fmoc was removed with piperidine (20%) in DMF and then coupled with yl)sulfonyl)-L-arginine according to SP-5 using PyBrop instead of HATU. The Fmoc protecting group was removed with piperidine (20%) in DMF and then coupled with 2,2′,2″-(10-(2-((2,5-dioxopyrrolidin-1-yl)oxy)-2-oxoethyl)-1,4,7,10-tetraazacyclododecane-1,4,7-triyl)triacetic acid (2.0 eq.) in anhydrous DMF and 10 eq. of TEA. All the protecting groups were removed according to SP-6 to afford the title compound (15 mg, 22%) as a white powder. ESI/MS (m/z): 1011 (M−H₂O+H).

### Preparation 191

### 2,2′,2″-(10-(2-(((S)-17-(4-(((R)-1-((2R,4S)-2-Borono-4-hydroxypyrrolidin-1-yl)-1-oxopropan-2-yl)carbamoyl)naphthalen-1-yl)-3-imino-2,16-dimethyl-9,17-dioxo-2,4,10,16-tetraazaheptadecan-8-yl)amino)-2-oxoethyl)-1,4,7,10-tetraazacyclododecane-1,4,7-triyl)triacetic acid

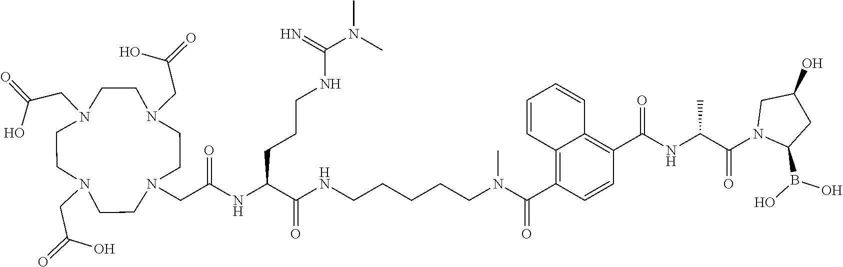 [SMILES: C[C@@H](NC(=O)c1ccc(C(=O)N(C)CCCCCNC(=O)[C@H](CCCNC(=N)N(C)C)NC(=O)CN2CCN(CC(=O)O)CCN(CC(=O)O)CCN(CC(=O)O)CC2)c2ccccc12)C(=O)N1C[C@@H](O)C[C@H]1B(O)O]

4-(Methoxycarbonyl)-1-naphthoic acid was coupled with 5-(methylamino)-N-Boc-pentanamine according to SP-5. The methyl ester was hydrolyzed with 0.5N NaOH in MeOH/THF and then coupled with H-D-Al(2R)-2-amino-1-((2R,4S)-4-hydroxy-2-((3aS,4S,6S)-3a,5,5-trimethylhexahydro-4,6-methanobenzo[d][1,3,2]dioxaborol-2-yl)pyrrolidin-1-yl)propan-1-one hydrochloride according to SP-5. The Boc protecting group was removed with TFA (20%) in DCM and was coupled with (E)-N²-(((9H-fluoren-9-yl)methoxy)carbonyl)-N^{w},N^{w}-dimethyl-N^{w′}-((2,2,4,6,7-pentamethyl-2,3-dihydrobenzofuran-5-yl)sulfonyl)-L-arginine according to SP-5 using PyBrop instead of HATU. The Fmoc protecting group was removed with piperidine (20%) in DMF and was coupled with 2,2′,2″-(10-(2-((2,5-dioxopyrrolidin-1-yl)oxy)-2-oxoethyl)-1,4,7,10-tetraazacyclododecane-1,4,7-triyl)triacetic acid (2.0 eq.) in anhydrous DMF and 10 eq. of TEA. All the protecting groups were removed according to SP-6 to afford the title compound (42 mg, 30%) as a white powder. ESI/MS (m/z): 1051 (M−H₂O+H).

### Preparation 192

### 2,2′-(4-(2-Amino-2-oxoethyl)-10-((S)-1-(4-(((R)-1-((2R,4S)-2-borono-4-hydroxypyrrolidin-1-yl)-1-oxopropan-2-yl)carbamoyl)phenyl)-10-(3-(3,3-dimethylguanidino)propyl)-3,9,12,18,26-pentaoxo-2,8,11,19,25-pentaazaheptacosan-27-yl)-1,4,7,10-tetraazacyclododecane-1,7-diyl)diacetic acid

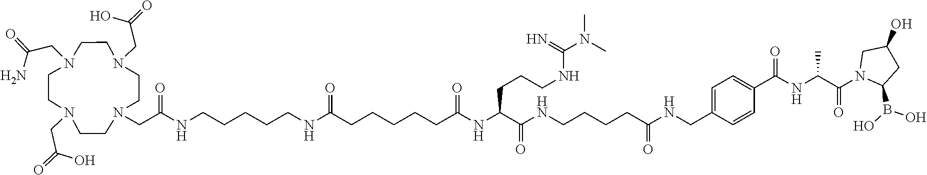 [SMILES: C[C@@H](NC(=O)c1ccc(CNC(=O)CCCCNC(=O)[C@H](CCCNC(=N)N(C)C)NC(=O)CCCCCC(=O)NCCCCCNC(=O)CN2CCN(CC(=O)O)CCN(CC(N)=O)CCN(CC(=O)O)CC2)cc1)C(=O)N1C[C@@H](O)C[C@H]1B(O)O]

The initial resin loading with 4-(((((9H-fluoren-9-yl)methoxy)carbonyl)amino)methyl)benzoic acid was performed as described in SP-1. The Fmoc protecting group was removed according to SP-3 and was coupled with 5-((((9H-fluoren-9-yl)methoxy)carbonyl)amino)pentanoic acid according to SP-2. The Fmoc protecting group was removed according to SP-3 and then coupled with (E)-N²-(((9H-fluoren-9-yl)methoxy)carbonyl)-N^{w},N^{w}-dimethyl-N^{w′}-((2,2,4,6,7-pentamethyl-2,3-dihydrobenzofuran-5-yl)sulfonyl)-L-arginine according to SP-2. The Fmoc protecting group was removed according to SP-3 and then coupled with 7-(4-nitrophenoxy)-7-oxoheptanoic acid in DMF and DIPEA. The free carboxylic acid was coupled to mono-(9H-fluoren-9-yl)methyl (5-aminopentyl)carbamate hydrochloride according to SP-2. The Fmoc protecting group was removed according to SP-3 and was coupled with 2-(7-(2-amino-2-oxoethyl)-4,10-bis(2-(tert-butoxy)-2-oxoethyl)-1,4,7,10-tetraazacyclododecan-1-yl)acetic acid according to SP-2. The peptide was cleaved from the resin according to SP-4 with TFA (5%) in DCM and then coupled with (2R)-2-amino-1-((2R,4S)-4-hydroxy-2-((3aS,4S,6S)-3a,5,5-trimethylhexahydro-4,6-methanobenzo[d][1,3,2]dioxaborol-2-yl)pyrrolidin-1-yl)propan-1-one hydrochloride according to SP-5. All the protecting groups were removed according to SP-6 to afford the title compound (10 mg, 6%). ESI/MS (m/z): 1213 (M−H₂O+H).

### Preparation 193

### (R)-5-(((S)-1-((5-(4-(((R)-1-((2R,4S)-2-Borono-4-hydroxypyrrolidin-1-yl)-1-oxopropan-2-yl)carbamoyl)-1-naphthamido)pentyl)amino)-5-guanidino-1-oxopentan-2-yl)amino)-5-oxo-2-(4,7,10-tris(2-amino-2-oxoethyl)-1,4,7,10-tetraazacyclododecan-1-yl)pentanoic acid

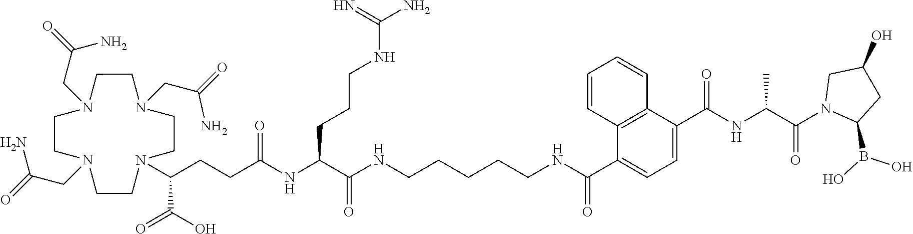 [SMILES: C[C@@H](NC(=O)c1ccc(C(=O)NCCCCCNC(=O)[C@H](CCCNC(=N)N)NC(=O)CC[C@H](C(=O)O)N2CCN(CC(N)=O)CCN(CC(N)=O)CCN(CC(N)=O)CC2)c2ccccc12)C(=O)N1C[C@@H](O)C[C@H]1B(O)O]

The initial 2-chlorotrityl chloride resin loading with naphthalene-1,4-dicarboxylic acid was performed as described in SP-1 and was coupled with mono-Fmoc-1, 5-diaminopentane hydrochloride according to SP-2 except using PyBrop as the coupling reagent. The Fmoc group was removed according to SP-3 and then coupled with N2-(((9H-fluoren-9-yl)methoxy)carbonyl)-N¹-((2,2,4,6,7-pentamethyl-2,3-dihydrobenzofuran-5-yl)sulfonyl)-L-arginine according to SP-2 using PyBrop as the coupling reagent. The amino group was removed according to SP-3 and then coupled with (R)-5-(tert-butoxy)-5-oxo-4-(4,7,10-tris(2-amino-2-oxoethyl)-1,4,7,10-tetraazacyclododecan-1-yl)pentanoic acid according to SP-2 using TBTU and HOBt as the coupling reagents. The peptide was cleaved from the resin according to SP-4 and coupled with (2R)-2-amino-1-((2R,4S)-4-hydroxy-2-((3aS,4S,6S)-3a,5,5-trimethylhexahydro-4,6-methanobenzo[d][1,3,2]dioxaborol-2-yl)pyrrolidin-1-yl)propan-1-one hydrochloride according to SP-5. The protecting groups were removed according to SP-6 to afford the title compound (6.6 mg, 5% yield at 0.1 mmol scale). ESI/MS (m/z): 1097 (M+H).

The following compounds in Table 23 were synthesized in essentially the same manner as described for (R)-5-(((S)-1-((5-(4-(((R)-1-((2R,4S)-2-borono-4-hydroxypyrrolidin-1-yl)-1-oxopropan-2-yl)carbamoyl)-1-naphthamido)pentyl)amino)-5-guanidino-1-oxopentan-2-yl)amino)-5-oxo-2-(4,7,10-tris(2-amino-2-oxoethyl)-1,4,7,10-tetraazacyclododecan-1-yl)pentanoic acid.

TABLE 23

| | | ESI/MS |
|---|---|---|
| Prep # | Structure | m/z |
| 194 | 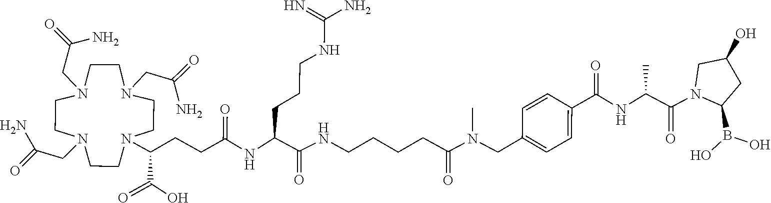 [SMILES: C[C@@H](NC(=O)c1ccc(CN(C)C(=O)CCCCNC(=O)[C@H](CCCNC(=N)N)NC(=O)CC[C@H](C(=O)O)N2CCN(CC(N)=O)CCN(CC(N)=O)CCN(CC(N)=O)CC2)cc1)C(=O)N1C[C@@H](O)C[C@H]1B(O)O] | 1043 (M − H₂O + H) |
| | (R)-5-(((S)-1-((5-((4-(((R)-1-((2R,4S)-2-borono-4-hydroxypyrrolidin-1-yl)-1- | |
| | oxopropan-2-yl)carbamoyl)benzyl)(methyl)amino)-5-oxopentyl)amino)-5- | |
| | guanidino-1-oxopentan-2-yl)amino)-5-oxo-2-(4,7,10-tris(2-amino-2-oxoethyl)- | |
| | 1,4,7,10-tetraazacyclododecan-1-yl)pentanoic acid | |
| 195 | 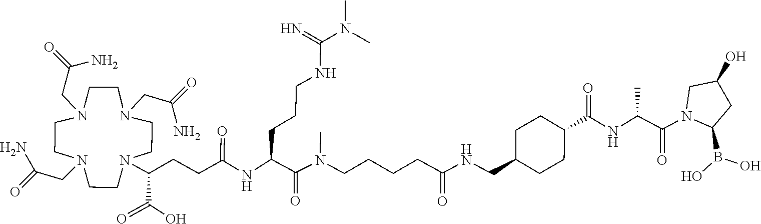 [SMILES: C[C@@H](NC(=O)[C@H]1CC[C@H](CNC(=O)CCCCN(C)C(=O)[C@H](CCCNC(=N)N(C)C)NC(=O)CC[C@H](C(=O)O)N2CCN(CC(N)=O)CCN(CC(N)=O)CCN(CC(N)=O)CC2)CC1)C(=O)N1C[C@@H](O)C[C@H]1B(O)O] | 1077 (M − H₂O + H) |
| | (R)-5-(((S)-17-((1R,4S)-4-(((R)-1-((2R,4S)-2-Borono-4-hydroxypyrrolidin-1-yl)- | |
| | 1-oxopropan-2-yl)carbamoyl)cyclohexyl)-3-imino-2,10-dimethyl-9,15-dioxo- | |
| | 2,4,10,16-tetraazaheptadecan-8-yl)amino)-5-oxo-2-(4,7,10-tris(2-amino-2- | |
| | oxoethyl)-1,4,7,10-tetraazacyclododecan-1-yl)pentanoic acid | |
| 196 | 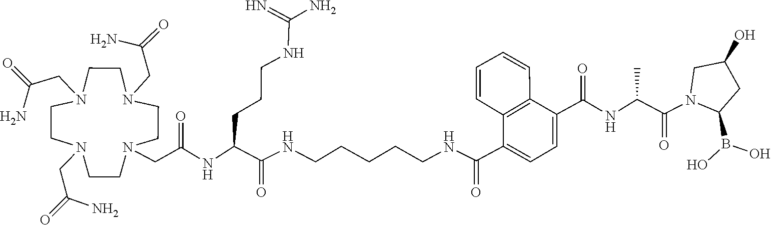 [SMILES: C[C@@H](NC(=O)c1ccc(C(=O)NCCCCCNC(=O)[C@H](CCCNC(=N)N)NC(=O)CN2CCN(CC(N)=O)CCN(CC(N)=O)CCN(CC(N)=O)CC2)c2ccccc12)C(=O)N1C[C@@H](O)C[C@H]1B(O)O] | 1025 (M + H) |
| | ((2R,4S)-1-((4-((5-((S)-5-Guanidino-2-(2-(4,7,10-tris(2-amino-2-oxoethyl)- | |
| | 1,4,7,10-tetraazacyclododecan-1-yl)acetamido)pentanamido)pentyl)carbamoyl)-1- | |
| | naphthoyl)-D-alanyl)-4-hydroxypyrrolidin-2-yl)boronic acid | |
| 197 | 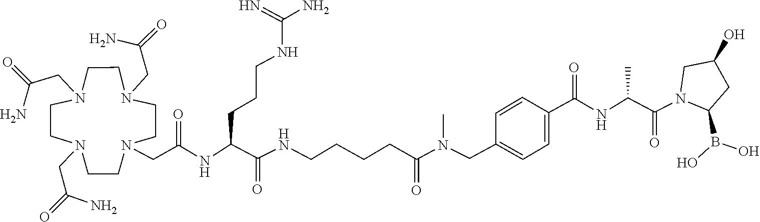 [SMILES: C[C@@H](NC(=O)c1ccc(CN(C)C(=O)CCCCNC(=O)[C@H](CCCNC(=N)N)NC(=O)CN2CCN(CC(N)=O)CCN(CC(N)=O)CCN(CC(N)=O)CC2)cc1)C(=O)N1C[C@@H](O)C[C@H]1B(O)O] | 989 (M + H) |
| | ((2R,4S)-1-((4-((5-((S)-5-Guanidino-2-(2-(4,7,10-tris(2-amino-2-oxoethyl)- | |
| | 1,4,7,10-tetraazacyclododecan-1-yl)acetamido)pentanamido)-N- | |
| | methylpentanamido)methyl)benzoyl)-D-alanyl)-4-hydroxypyrrolidin-2-yl)boronic | |
| | acid | |
| 198 | 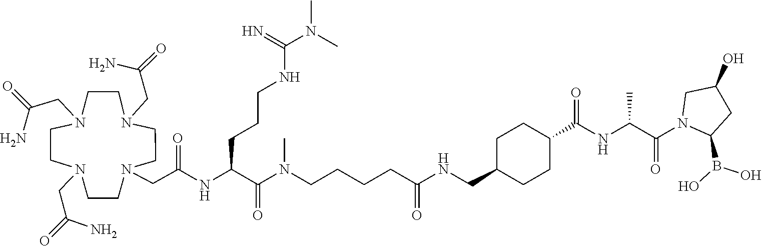 [SMILES: C[C@@H](NC(=O)[C@H]1CC[C@H](CNC(=O)CCCCN(C)C(=O)[C@H](CCCNC(=N)N(C)C)NC(=O)CN2CCN(CC(N)=O)CCN(CC(N)=O)CCN(CC(N)=O)CC2)CC1)C(=O)N1C[C@@H](O)C[C@H]1B(O)O] | 1023 (M + H) |
| | ((2R,4S)-4-hydroxy-1-(((1S,4R)-4-((S)-3-imino-2,10-dimethyl-9,15-dioxo-8-(2- | |
| | (4,7,10-tris(2-amino-2-oxoethyl)-1,4,7,10-tetraazacyclododecan-1-yl)acetamido)- | |
| | 2,4,10,16-tetraazaheptadecan-17-yl)cyclohexane-1-carbonyl)-D-alanyl)pyrrolidin- | |
| | 2-yl)boronic acid | |

### Preparation 199

### (8S,14S)-14-((4-(((R)-1-((2R,4S)-2-Borono-4-hydroxypyrrolidin-1-yl)-1-oxopropan-2-yl)carbamoyl)benzyl)carbamoyl)-8-((R)-4-carboxy-4-(4,7,10-tris(2-amino-2-oxoethyl)-1,4,7,10-tetraazacyclododecan-1-yl)butanamido)-3-imino-2-methyl-9,12-dioxo-2,4,10,13-tetraazahexadecan-16-oic acid

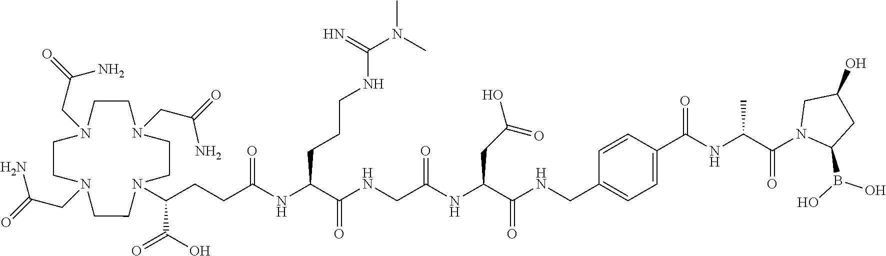 [SMILES: C[C@@H](NC(=O)c1ccc(CNC(=O)[C@H](CC(=O)O)NC(=O)CNC(=O)[C@H](CCCNC(=N)N(C)C)NC(=O)CC[C@H](C(=O)O)N2CCN(CC(N)=O)CCN(CC(N)=O)CCN(CC(N)=O)CC2)cc1)C(=O)N1C[C@@H](O)C[C@H]1B(O)O]

The initial 2-chlorotrityl chloride resin loading with 4-(N-Fmoc-aminomethyl)benzoic acid was performed as described in SP-1. The Fmoc group was removed according to SP-3 and then coupled with (S)-2-((((9H-fluoren-9-yl)methoxy)carbonyl)amino)-4-(tert-butoxy)-4-oxobutanoic acid according to SP-2. The Fmoc group was removed according to SP-3 and then coupled with (((9H-fluoren-9-yl)methoxy)carbonyl)glycine according to SP-2. The Fmoc group was removed according to SP-3 and coupled (S,Z)-2-((((9H-fluoren-9-yl)methoxy)carbonyl)amino)-5-(((dimethylamino)((2,2,4,6,7-pentamethyl-2,3-dihydrobenzofuran)-5-sulfonamido)methylene)amino)pentanoic acid according to SP-2. The Fmoc group was removed according to SP-3 and coupled with (R)-5-(tert-butoxy)-5-oxo-4-(4,7,10-tris(2-amino-2-oxoethyl)-1,4,7,10-tetraazacyclododecan-1-yl)pentanoic acid according to SP-2 using TBTU and HOBt as the coupling reagents. The peptide was cleaved from the resin according to SP-4 and coupled with (2R)-2-amino-1-((2R,4S)-4-hydroxy-2-((3aS,4S,6S)-3a,5,5-trimethylhexahydro-4,6-methanobenzo[d][1,3,2]dioxaborol-2-yl)pyrrolidin-1-yl)propan-1-one hydrochloride according to SP-5. The remaining protecting groups were removed according to SP-6 to give the title compound (14 mg, 9% yield at 0.1 mmol scale). ESI/MS (m/z): 1130 (M−H₂O+H).

The following compound in Table 24 was synthesized in essentially the same manner as described for (8S,14S)-14-((4-(((R)-1-((2R,4S)-2-Borono-4-hydroxypyrrolidin-1-yl)-1-oxopropan-2-yl)carbamoyl)benzyl)carbamoyl)-8-((R)-4-carboxy-4-(4,7,10-tris(2-amino-2-oxoethyl)-1,4,7,10-tetraazacyclododecan-1-yl)butanamido)-3-imino-2-methyl-9,12-dioxo-2,4,10,13-tetraazahexadecan-16-oic acid.

TABLE 24

| Prep | | |
|---|---|---|
| # | Structure | ESI/MS m/z |
| 200 | 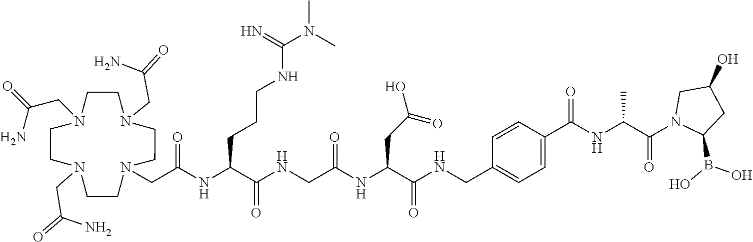 [SMILES: C[C@@H](NC(=O)c1ccc(CNC(=O)[C@H](CC(=O)O)NC(=O)CNC(=O)[C@H](CCCNC(=N)N(C)C)NC(=O)CN2CCN(CC(N)=O)CCN(CC(N)=O)CCN(CC(N)=O)CC2)cc1)C(=O)N1C[C@@H](O)C[C@H]1B(O)O] | 1076 (M + H) |
| | (8S,14S)-14-((4-(((R)-1-((2R,4S)-2-Borono-4-hydroxypyrrolidin-1-yl)-1- | |
| | oxopropan-2-yl)carbamoyl)benzyl)carbamoyl)-3-imino-2-methyl-9,12-dioxo-8- | |
| | (2-(4,7,10-tris(2-amino-2-oxoethyl)-1,4,7,10-tetraazacyclododecan-1- | |
| | yl)acetamido)-2,4,10,13-tetraazahexadecan-16-oic acid | |

### EXAMPLES

### Example 1

### Lead(II) 2,2′-(4-(2-amino-2-oxoethyl)-10-(2-(((S)-1-((5-(4-(((R)-1-((R)-2-boronopyrrolidin-1-yl)-1-oxopropan-2-yl)carbamoyl)-1-naphthamido)pentyl)amino)-5-guanidino-1-oxopentan-2-yl)amino)-2-oxoethyl)-1,4,7,10-tetraazacyclododecane-1,7-diyl)diacetate

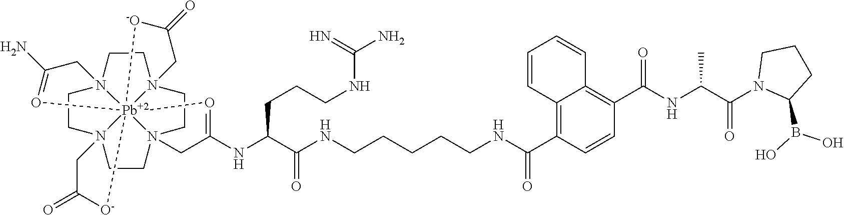

2,2′-(4-(2-Amino-2-oxoethyl)-10-(2-(((S)-1-((5-(4-(((R)-1-((R)-2-boronopyrrolidin-1-yl)-1-oxopropan-2-yl)carbamoyl)-1-naphthamido)pentyl)amino)-5-guanidino-1-oxopentan-2-yl)amino)-2-oxoethyl)-1,4,7,10-tetraazacyclododecane-1,7-diyl)diacetic acid (5.1 mg, 0.004 mmol) was reacted with Pb(NO₃)₂ according to SP-7 to afford the title compound (4.1 mg, 70%) as a white powder. ESI/MS m/z: 1217 (M+H).

The following compounds in Table 24 were synthesized in essentially the same manner as described for ((2R)-1-((4-((5-((2S)-2-(2-(17-(2-amino-2-oxoethyl)-3,7-dioxo-4,6-dioxa-1,9,12,17-tetraaza-5l²-plumbabicyclo[7.5.5]nonadecan-12-yl)acetamido)-5-guanidinopentanamido)pentyl)carbamoyl)-1-naphthoyl)-D-alanyl)pyrrolidin-2-yl)boronic acid

TABLE 24

| Ex | | |
|---|---|---|
| # | Structure | ESI/MS m/z |
| 2 | 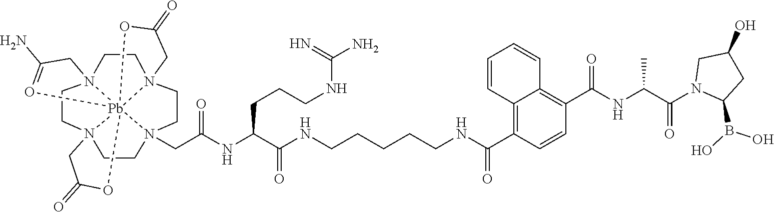 | 1233 (M + H) |
| | Lead(II) 2,2′-(4-(2-amino-2-oxoethyl)-10-(2-(((S)-1-((5-(4-(((R)-1-((2R,4S)- | |
| | 2-borono-4-hydroxypyrrolidin-1-yl)-1-oxopropan-2-yl)carbamoyl)-1- | |
| | naphthamido)pentyl)amino)-5-guanidino-1-oxopentan-2-yl)amino)-2- | |
| | oxoethyl)-1,4,7,10-tetraazacyclododecane-1,7-diyl)diacetate | |
| 3 | 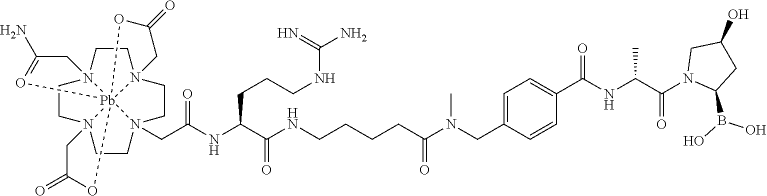 | 1195 (M + H) |
| | Lead(II) 2,2′-(4-(2-amino-2-oxoethyl)-10-(2-(((S)-1-((5-((4-(((R)-1- | |
| | ((2R,4S)-2-borono-4-hydroxypyrrolidin-1-yl)-1-oxopropan-2- | |
| | yl)carbamoyl)benzyl)(methyl)amino)-5-oxopentyl)amino)-5-guanidino-1- | |
| | oxopentan-2-yl)amino)-2-oxoethyl)-1,4,7,10-tetraazacyclododecane-1,7- | |
| | diyl)diacetate | |
| 4 | 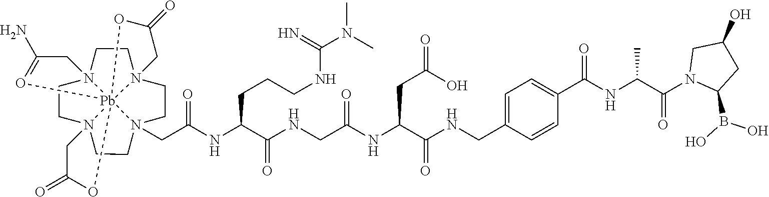 | 1266 [M − H₂O + H] |
| | Lead(II) 2,2′-(4-(2-amino-2-oxoethyl)-10-((4S,10S)-1-(4-(((R)-1-((2R,4S)-2- | |
| | borono-4-hydroxypyrrolidin-1-yl)-1-oxopropan-2-yl)carbamoyl)phenyl)-4- | |
| | (carboxymethyl)-10-(3-(3,3-dimethylguanidino)propyl)-3,6,9,12-tetraoxo- | |
| | 2,5,8,11-tetraazatridecan-13-yl)-1,4,7,10-tetraazacyclododecane-1,7- | |
| | diyl)diacetate | |
| 5 | 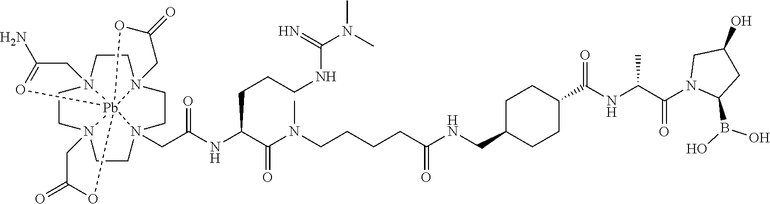 | 1213 [M − H₂O + H] |
| | Lead(II) 2,2′-(4-(2-amino-2-oxoethyl)-10-(2-(((S)-1-((((1R,4S)-4-(((R)- | |
| | 1-((2R,4S)-2-borono-4-hydroxypyrrolidin-1-yl)-1-oxopropan-2- | |
| | yl)carbamoyl)cyclohexyl)methyl)amino)-5-oxopentyl)amino)-5-guanidino- | |
| | 1-oxopentan-2-yl)amino)-2-oxoethyl)-1,4,7,10-tetraazacyclododecane-1,7- | |
| | diyl)diacetate | |
| 41 | 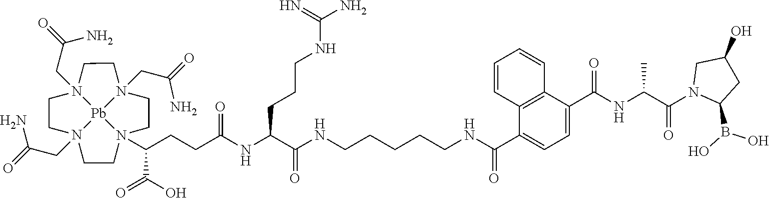 | 1417 (M + TFA) |
| | Lead(II) (R)-5-(((S)-1-((5-(4-(((R)-1-((2R,4S)-2-borono-4- | |
| | hydroxypyrrolidin-1-yl)-1-oxopropan-2-yl)carbamoyl)-1- | |
| | naphthamido)pentyl)amino)-5-guanidino-1-oxopentan-2-yl)amino)-5-oxo-2- | |
| | (4,7,10-tris(2-amino-2-oxoethyl)-1,4,7,10-tetraazacyclododecan-1- | |
| | yl)pentanoic acid | |
| 42 | 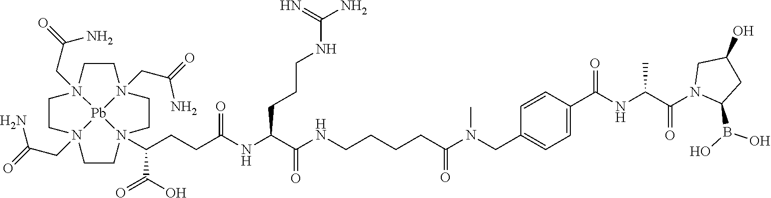 | 1381 (M + TFA) |
| | Lead(II) (R)-5-(((S)-1-((5-((4-(((R)-1-((2R,4S)-2-borono-4- | |
| | hydroxypyrrolidin-1-yl)-1-oxopropan-2- | |
| | yl)carbamoyl)benzyl)(methyl)amino)-5-oxopentyl)amino)-5-guanidino-1- | |
| | oxopentan-2-yl)amino)-5-oxo-2-(4,7,10-tris(2-amino-2-oxoethyl)-1,4,7,10- | |
| | tetraazacyclododecan-1-yl)pentanoic acid | |
| 43 | 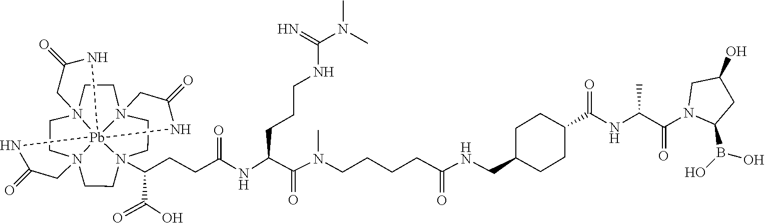 | 1415 (M + TFA) |
| | Lead(II) (R)-5-(((S)-17-((1R,4S)-4-(((R)-1-((2R,4S)-2-borono-4- | |
| | hydroxypyrrolidin-1-yl)-1-oxopropan-2-yl)carbamoyl)cyclohexyl)-3-imino- | |
| | 2,10-dimethyl-9,15-dioxo-2,4,10,16-tetraazaheptadecan-8-yl)amino)-5-oxo- | |
| | 2-(4,7,10-tris(2-amino-2-oxoethyl)-1,4,7,10-tetraazacyclododecane-1- | |
| | yl)pentanoic acid | |
| 44 | 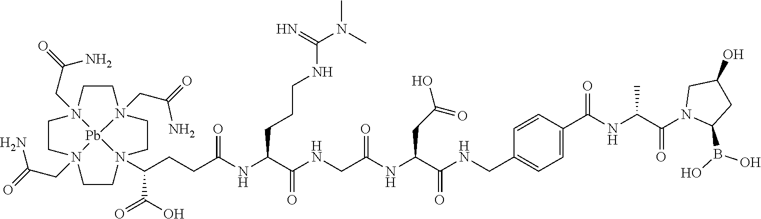 | 678 (M/2 + H) |
| | Lead(II) (8S,14S)-14-((4-(((R)-1-((2R,4S)-2-borono-4-hydroxypyrrolidin-1- | |
| | yl)-1-oxopropan-2-yl)carbamoyl)benzyl)carbamoyl)-8-((R)-4-carboxy-4- | |
| | (4,7,10-tris(2-amino-2-oxoethyl)-1,4,7,10-tetraazacyclododecan-1- | |
| | yl)butanamido)-3-imino-2-methyl-9,12-dioxo-2,4,10,13-tetraazahexadecan- | |
| | 16-oic acid | |
| 45 | 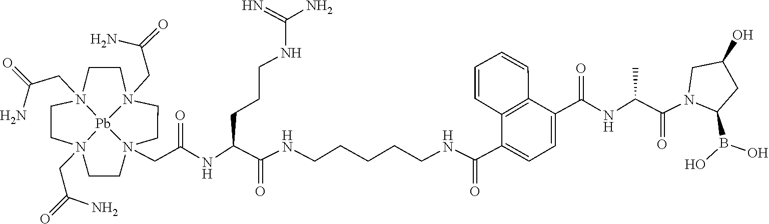 | 1458 (M + 2TFA) |
| | Lead(II) ((2R,4S)-1-((4-((5-((S)-5-guanidino-2-(2-(4,7,10-tris(2-amino-2- | |
| | oxoethyl)-1,4,7,10-tetraazacyclododecan-1- | |
| | yl)acetamido)pentanamido)pentyl)carbamoyl)-1-naphthoyl)-D-alanyl)-4- | |
| | hydroxypyrrolidin-2-yl)boronic acid | |
| 46 | 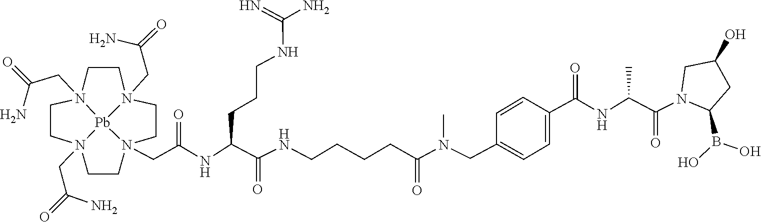 | 1422 (M + 2TFA) |
| | Lead(II) ((2R,4S)-1-((4-((5-((S)-5-guanidino-2-(2-(4,7,10-tris(2-amino-2- | |
| | oxoethyl)-1,4,7,10-tetraazacyclododecan-1-yl)acetamido)pentanamido)-N- | |
| | methylpentanamido)methyl)benzoyl)-D-alanyl)-4-hydroxypyrrolidin-2- | |
| | yl)boronic acid | |
| 47 | 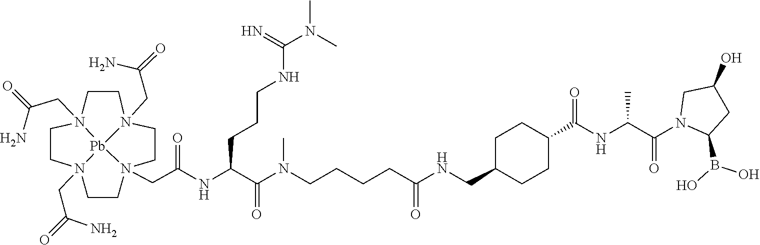 | 1457 (M + 2TFA) |
| | Lead(II) ((2R,4S)-4-hydroxy-1-(((1S,4R)-4-((S)-3-imino-2,10-dimethyl- | |
| | 9,15-dioxo-8-(2-(4,7,10-tris(2-amino-2-oxoethyl)-1,4,7,10- | |
| | tetraazacyclododecan-1-yl)acetamido)-2,4,10,16-tetraazaheptadecan-17- | |
| | yl)cyclohexane-1-carbonyl)-D-alanyl)pyrrolidin-2-yl)boronic acid | |
| 48 | 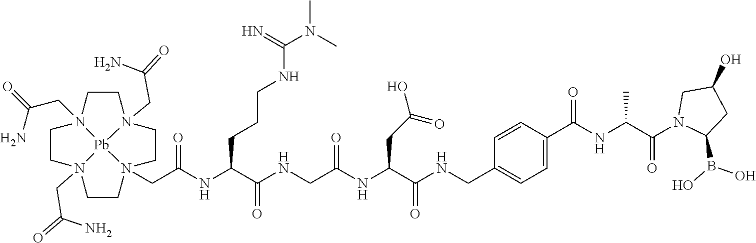 | 697 (M + TFA)/2) |
| | Lead(II) (8S,14S)-14-((4-(((R)-1-((2R,4S)-2-borono-4-hydroxypyrrolidin-1- | |
| | yl)-1-oxopropan-2-yl)carbamoyl)benzyl)carbamoyl)-3-imino-2-methyl-9,12- | |
| | dioxo-8-(2-(4,7,10-tris(2-amino-2-oxoethyl)-1,4,7,10-tetraazacyclododecan- | |
| | 1-yl)acetamido)-2,4,10,13-tetraazahexadecan-16-oic acid | |

### Example 6

Lutetium(III) 2,2′,2″-(10-(2-(((S)-1-((5-((4-(((R)-1-((2R,4S)-2-borono-4-hydroxypyrrolidin-1-yl)-1-oxopropan-2-yl)carbamoyl)benzyl)(methyl)amino)-5-oxopentyl)amino)-5-guanidino-1-oxopentan-2-yl)amino)-2-oxoethyl)-1,4,7,10-tetraazacyclododecane-1,4,7-triyl)triacetate

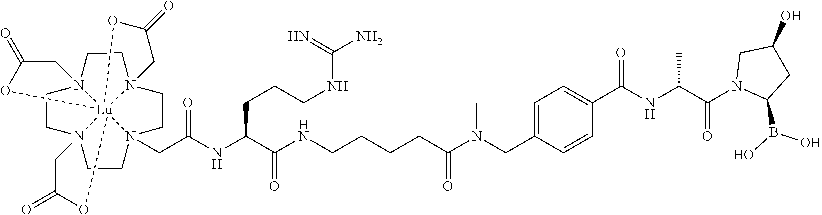

2,2′,2″-(10-(2-(((S)-1-((5-((4-(((R)-1-((2R,4S)-2-Borono-4-hydroxypyrrolidin-1-yl)-1-oxopropan-2-yl)carbamoyl)benzyl)(methyl)amino)-5-oxopentyl)amino)-5-guanidino-1-oxopentan-2-yl)amino)-2-oxoethyl)-1,4,7,10-tetraazacyclododecane-1,4,7-triyl)triacetic acid (7.4 mg, 0.005 mmol) was reacted with LuCl₃ according to SP-8 to afford the title compound as a white powder (5.4 mg, 65%). ESI/MS m/z: 1164 (M+H).

The following compounds in Table 25 were synthesized in essentially the same manner as described for lutetium(III) 2,2′,2″-(10-(2-(((S)-1-((5-((4-(((R)-1-((2R,4S)-2-borono-4-hydroxypyrrolidin-1-yl)-1-oxopropan-2-yl)carbamoyl)benzyl)(methyl)amino)-5-oxopentyl)amino)-5-guanidino-1-oxopentan-2-yl)amino)-2-oxoethyl)-1,4,7,10-tetraazacyclododecane-1,4,7-triyl)triacetate.

TABLE 25

| Ex | | |
|---|---|---|
| # | Structure | ESI/MS |
| 7 | 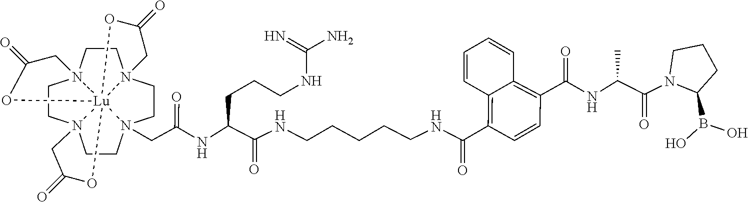 | 1184 (M + H) |
| | Lutetium(III) 2,2′,2″-(10-(2-(((S)-1-((5-(4-(((R)-1-((R)-2-boronopyrrolidin- | |
| | 1-yl)-1-oxopropan-2-yl)carbamoyl)-1-naphthamido)pentyl)amino)-5- | |
| | guanidino-1-oxopentan-2-yl)amino)-2-oxoethyl)-1,4,7,10- | |
| | tetraazacyclododecane-1,4,7-triyl)triacetate | |
| 8 | 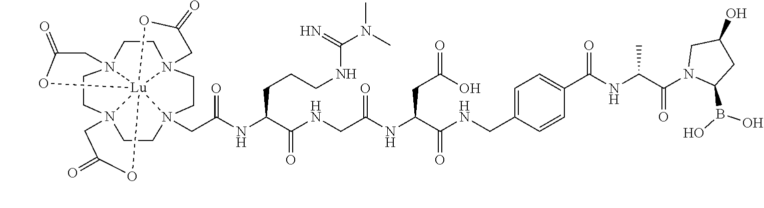 | 1250 (M + H) |
| | Lutetium(III) 2,2′,2″-(10-((4S,10S)-1-(4-(((R)-1-((2R,4S)-2-borono-4- | |
| | hydroxypyrrolidin-1-yl)-1-oxopropan-2-yl)carbamoyl)phenyl)-4- | |
| | (carboxymethyl)-10-(3-(3,3-dimethylguanidino)propyl)-3,6,9,12-tetraoxo- | |
| | 2,5,8,11-tetraazatridecan-13-yl)-1,4,7,10-tetraazacyclododecane-1,4,7- | |
| | triyl)triacetate | |
| 9 | 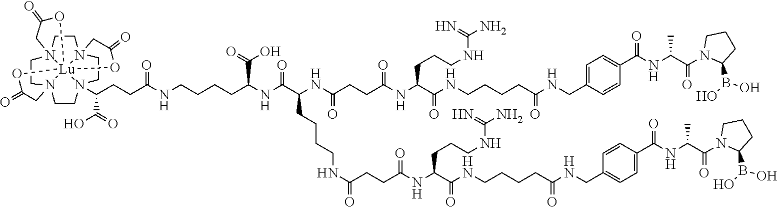 | 1083 (M − 3H₂O/2 + H) |
| | Lutetium(III) 2,2′-(7-((10S,21S,24S,33R)-21-(4-(((S)-1-((5-((4-(((R)-1-((R)- | |
| | 2-boronopyrrolidin-1-yl)-1-oxopropan-2-yl)carbamoyl)benzyl)amino)-5- | |
| | oxopentyl)amino)-5-guanidino-1-oxopentan-2-yl)amino)-4-oxobutanamido)- | |
| | 1-(4-(((R)-1-((R)-2-boronopyrrolidin-1-yl)-1-oxopropan-2- | |
| | yl)carbamoyl)phenyl)-24-carboxy-33-carboxylato-10-(3-guanidinopropyl)- | |
| | 3,9,12,15,22,30-hexaoxo-2,8,11,16,23,29-hexaazatritriacontan-33-yl)-10- | |
| | (carboxymethyl)-1,4,7,10-tetraazacyclododecane-1,4-diyl)diacetate | |
| 10 | 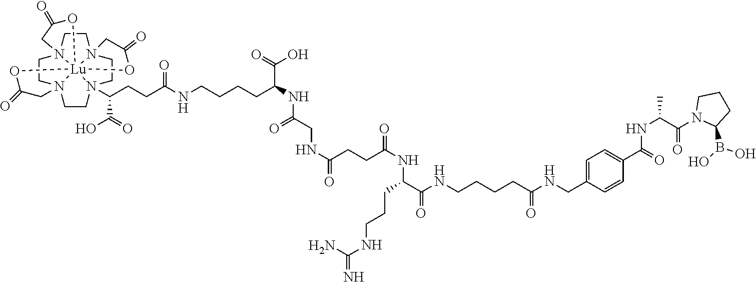 | 1473 (M − H₂O + H) |
| | Lutetium(III) 2,2′-(7-((10S,20S,29R)-1-(4-(((R)-1-((R)-2-boronopyrrolidin- | |
| | 1-yl)-1-oxopropan-2-yl)carbamoyl)phenyl)-20-carboxy-29-carboxylato-10- | |
| | (3-guanidinopropyl)-3,9,12,15,18,26-hexaoxo-2,8,11,16,19,25- | |
| | hexaazanonacosan-29-yl)-10-(carboxymethyl)-1,4,7,10- | |
| | tetraazacyclododecane-1,4-diyl)diacetate | |
| 11 | 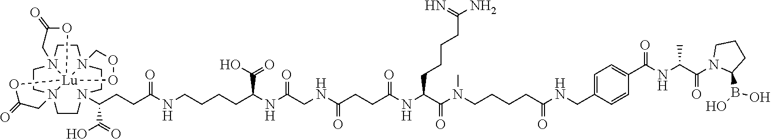 | 1487 (M − H₂O + H) |
| | Lutetium(III) 2,2′,2″-(10-((10S,20S,29R)-1-(4-(((R)-1-((R)-2- | |
| | boronopyrrolidin-1-yl)-1-oxopropan-2-yl)carbamoyl)phenyl)-20,29- | |
| | dicarboxy-10-(3-guanidinopropyl)-8-methyl-3,9,12,15,18,26-hexaoxo- | |
| | 2,8,11,16,19,25-hexaazanonacosan-29-yl)-1,4,7,10-tetraazacyclododecane- | |
| | 1,4,7-triyl)triacetate | |
| 12 | 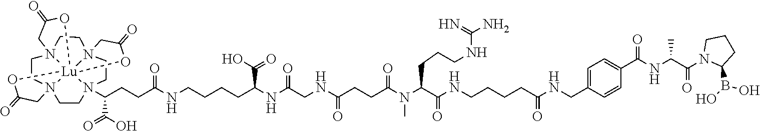 | 1487 (M − H₂O + H) |
| | Lutetium(III) 2,2′,2″-(10-((10S,20S,29R)-1-(4-(((R)-1-((R)-2- | |
| | boronopyrrolidin-1-yl)-1-oxopropan-2-yl)carbamoyl)phenyl)-20,29- | |
| | dicarboxy-10-(3-guanidinopropyl)-11-methyl-3,9,12,15,18,26-hexaoxo- | |
| | 2,8,11,16,19,25-hexaazanonacosan-29-yl)-1,4,7,10-tetraazacyclododecane- | |
| | 1,4,7-triyl)triacetate | |
| 13 | 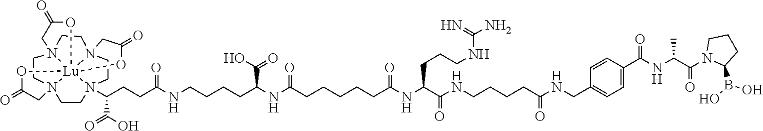 | 1458 (M − H₂O + H) |
| | Lutetium(III) 2,2′,2″-(10-((10S,20S,29R)-1-(4-(((R)-1-((R)-2- | |
| | boronopyrrolidin-1-yl)-1-oxopropan-2-yl)carbamoyl)phenyl)-20,29- | |
| | dicarboxy-10-(3-guanidinopropyl)-3,9,12,18,26-pentaoxo-2,8,11,19,25- | |
| | pentaazanonacosan-29-yl)-1,4,7,10-tetraazacyclododecane-1,4,7- | |
| | triyl)triacetate | |
| 14 | 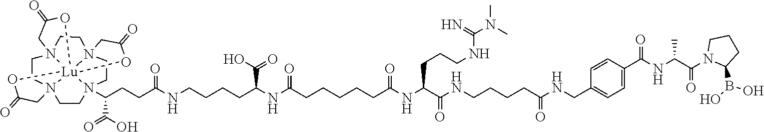 | 1486 (M − H₂O + H) |
| | Lutetium(III) 2,2′,2″-(10-((10S,20S,29R)-1-(4-(((R)-1-((R)-2- | |
| | boronopyrrolidin-1-yl)-1-oxopropan-2-yl)carbamoyl)phenyl)-20,29- | |
| | dicarboxy-10-(3-(3,3-dimethylguanidino)propyl)-3,9,12,18,26-pentaoxo- | |
| | 2,8,11,19,25-pentaazanonacosan-29-yl)-1,4,7,10-tetraazacyclododecane- | |
| | 1,4,7-triyl)triacetate | |
| 15 | 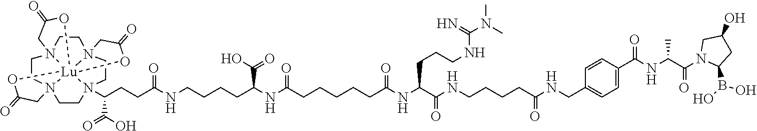 | 1502 (M − H₂O + H) |
| | Lutetium(III) 2,2′,2″-(10-((10S,20S,29R)-1-(4-(((R)-1-((2R,4S)-2-borono-4- | |
| | hydroxypyrrolidin-1-yl)-1-oxopropan-2-yl)carbamoyl)phenyl)-20,29- | |
| | dicarboxy-10-(3-(3,3-dimethylguanidino)propyl)-3,9,12,18,26-pentaoxo- | |
| | 2,8,11,19,25-pentaazanonacosan-29-yl)-1,4,7,10-tetraazacyclododecane- | |
| | 1,4,7-triyl)triacetate | |
| 16 | 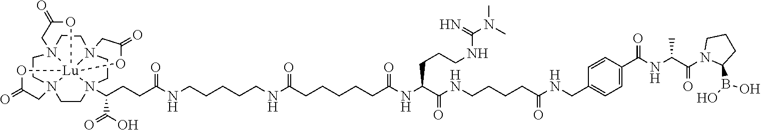 | 1442 (M − H₂O + H) |
| | Lutetium(III) 2,2′,2″-(10-((10S,29R)-1-(4-(((R)-1-((R)-2-boronopyrrolidin-1- | |
| | yl)-1-oxopropan-2-yl)carbamoyl)phenyl)-29-carboxy-10-(3-(3,3- | |
| | dimethylguanidino)propyl)-3,9,12,18,26-pentaoxo-2,8,11,19,25- | |
| | pentaazanonacosan-29-yl)-1,4,7,10-tetraazacyclododecane-1,4,7- | |
| | triyl)triacetate | |
| 17 | 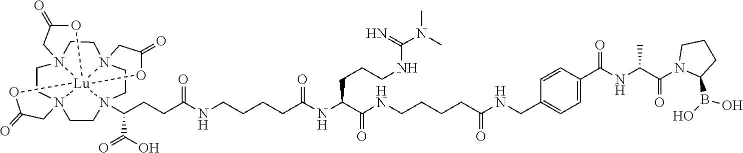 | 1315 (M − H₂O + H) |
| | Lutetium(III) 2,2′-(7-((10S,21R)-1-(4-(((R)-1-((R)-2-boronopyrrolidin-1-yl)- | |
| | 1-oxopropan-2-yl)carbamoyl)phenyl)-21-carboxylato-10-(3-(3,3- | |
| | dimethylguanidino)propyl)-3,9,12,18-tetraoxo-2,8,11,17-tetraazahenicosan- | |
| | 21-yl)-10-(carboxymethyl)-1,4,7,10-tetraazacyclododecane-1,4- | |
| | diyl)diacetate | |
| 18 | 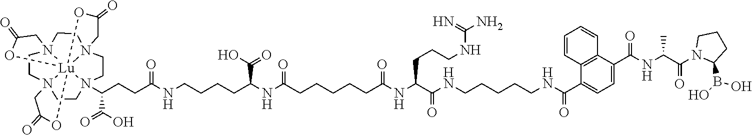 | 755 (M − H₂O/2 + H) |
| | Lutetium(III) 2,2′,2″-(10-((10S,20S,29R)-1-(4-(((R)-1-((R)-2- | |
| | boronopyrrolidin-1-yl)-1-oxopropan-2-yl)carbamoyl)naphthalen-1-yl)-20,29- | |
| | dicarboxy-10-(3-guanidinopropyl)-1,9,12,18,26-pentaoxo-2,8,11,19,25- | |
| | pentaazanonacosan-29-yl)-1,4,7,10-tetraazacyclododecane-1,4,7- | |
| | triyl)triacetate | |
| 19 | 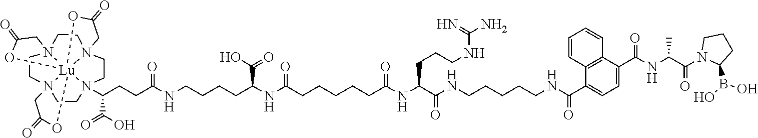 | 1436 (M − H₂O + H) |
| | Lutetium(III) 2,2′,2″-(10-((10S,20S)-1-(4-(((R)-1-((R)-2-boronopyrrolidin-1- | |
| | yl)-1-oxopropan-2-yl)carbamoyl)naphthalen-1-yl)-20-carboxy-10-(3- | |
| | guanidinopropyl)-1,9,12,18,26-pentaoxo-2,8,11,19,25-pentaazaheptacosan- | |
| | 27-yl)-1,4,7,10-tetraazacyclododecane-1,4,7-triyl)triacetate | |
| 20 | 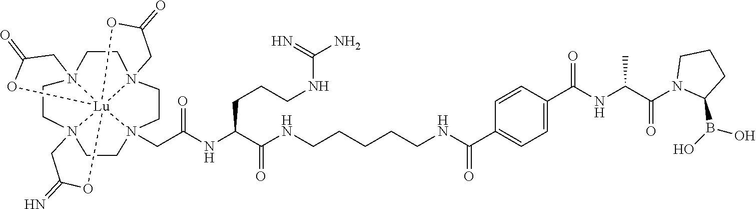 | 1134 (M + H) |
| | Lutetium(III) 2,2′,2″-(10-(2-(((S)-1-((5-((4-(((R)-1-((R)-2-boronopyrrolidin- | |
| | 1-yl)-1-oxopropan-2-yl)carbamoyl)benzyl)amino)-5-oxopentyl)amino)-5- | |
| | guanidino-1-oxopentan-2-yl)amino)-2-oxoethyl)-1,4,7,10- | |
| | tetraazacyclododecane-1,4,7-triyl)triacetate | |
| 21 | 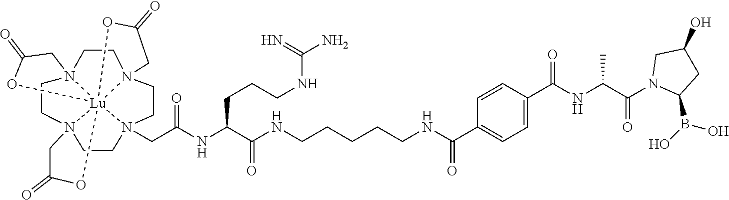 | 1150 (M + H) |
| | Lutetium(III) 2,2′,2″-(10-(2-(((S)-1-((5-((4-(((R)-1-((2R,4S)-2-borono-4- | |
| | hydroxypyrrolidin-1-yl)-1-oxopropan-2-yl)carbamoyl)benzyl)amino)-5- | |
| | oxopentyl)amino)-5-guanidino-1-oxopentan-2-yl)amino)-2-oxoethyl)- | |
| | 1,4,7,10-tetraazacyclododecane-1,4,7-triyl)triacetate | |
| 22 | 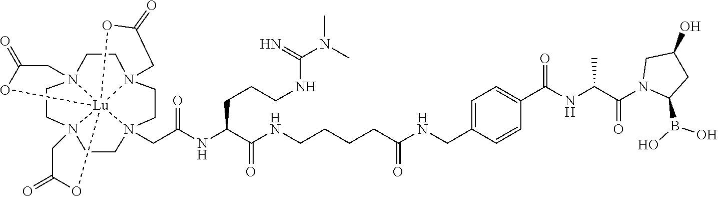 | 1178 (M + H) |
| | Lutetium(III) 2,2′,2″-(10-(2-(((S)-17-(4-(((R)-1-((2R,4S)-2-borono-4- | |
| | hydroxypyrrolidin-1-yl)-1-oxopropan-2-yl)carbamoyl)phenyl)-3-imino-2- | |
| | methyl-9,15-dioxo-2,4,10,16-tetraazaheptadecan-8-yl)amino)-2-oxoethyl)- | |
| | 1,4,7,10-tetraazacyclododecane-1,4,7-triyl)triacetate | |
| 23 | 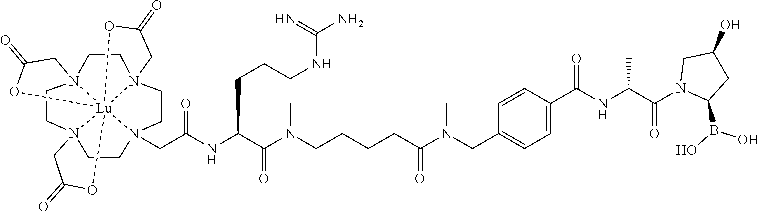 | 1178 (M + H) |
| | Lutetium(III) 2,2′,2″-(10-(2-(((S)-1-((5-((4-(((R)-1-((2R,4S)-2-borono-4- | |
| | hydroxypyrrolidin-1-yl)-1-oxopropan-2- | |
| | yl)carbamoyl)benzyl)(methyl)amino)-5-oxopentyl)(methyl)amino)-5- | |
| | guanidino-1-oxopentan-2-yl)amino)-2-oxoethyl)-1,4,7,10- | |
| | tetraazacyclododecane-1,4,7-triyl)triacetate | |
| 24 | 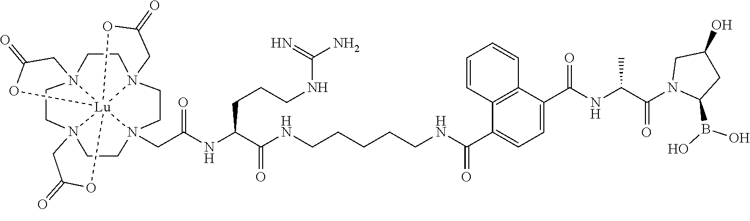 | 1200 (M + H) |
| | Lutetium(III) 2,2′,2″-(10-(2-(((S)-1-((5-(4-(((R)-1-((2R,4S)-2-borono-4- | |
| | hydroxypyrrolidin-1-yl)-1-oxopropan-2-yl)carbamoyl)-1- | |
| | naphthamido)pentyl)amino)-5-guanidino-1-oxopentan-2-yl)amino)-2- | |
| | oxoethyl)-1,4,7,10-tetraazacyclododecane-1,4,7-triyl)triacetate | |
| 25 | 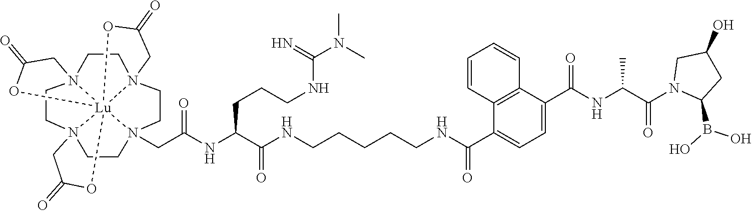 | 1228 (M + H) |
| | Lutetium(III) 2,2′,2″-(10-(2-(((S)-17-(4-(((R)-1-((R)-2-boronopyrrolidin-1- | |
| | yl)-1-oxopropan-2-yl)carbamoyl)naphthalen-1-yl)-3-imino-2-methyl-9,17- | |
| | dioxo-2,4,10,16-tetraazaheptadecan-8-yl)amino)-2-oxoethyl)-1,4,7,10- | |
| | tetraazacyclododecane-1,4,7-triyl)triacetate | |
| 26 | 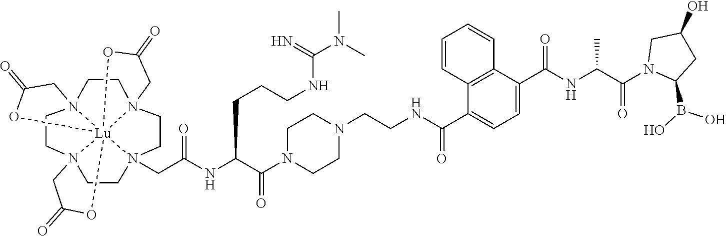 | 619 [(M − H₂O)/2] + H) |
| | Lutetium(III) 2,2′,2″-(10-(2-(((S)-1-(4-(2-((4-(((R)-1-((2R,4S)-2-borono-4- | |
| | hydroxypyrrolidin-1-yl)-1-oxopropan-2-yl)carbamoyl)benzyl)amino)-2- | |
| | oxoethyl)piperazin-1-yl)-5-(3,3-dimethylguanidino)-1-oxopentan-2- | |
| | yl)amino)-2-oxoethyl)-1,4,7,10-tetraazacyclododecane-1,4,7-triyl)triacetate | |
| 27 | 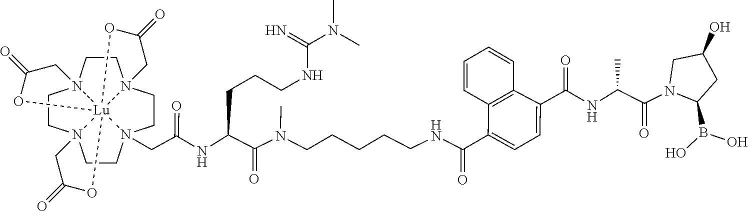 | 1242 (M + H) |
| | Lutetium(III) 2,2′,2″-(10-(2-(((S)-17-(4-(((R)-1-((R)-2-boronopyrrolidin-1- | |
| | yl)-1-oxopropan-2-yl)carbamoyl)naphthalen-1-yl)-3-imino-2,10-dimethyl- | |
| | 9,17-dioxo-2,4,10,16-tetraazaheptadecan-8-yl)amino)-2-oxoethyl)-1,4,7,10- | |
| | tetraazacyclododecane-1,4,7-triyl)triacetate | |
| 28 | 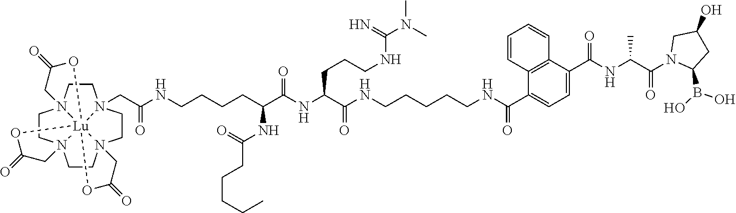 | 1454 (M + H) |
| | Lutetium(III) 2,2′,2″-(10-((10S,13S)-1-(4-(((R)-1-((2R,4S)-2-borono-4- | |
| | hydroxypyrrolidin-1-yl)-1-oxopropan-2-yl)carbamoyl)naphthalen-1-yl)-10- | |
| | (3-(3,3-dimethylguanidino)propyl)-13-hexanamido-1,9,12,19-tetraoxo- | |
| | 2,8,11,18-tetraazaicosan-20-yl)-1,4,7,10-tetraazacyclododecane-1,4,7- | |
| | triyl)triacetate | |
| 29 | 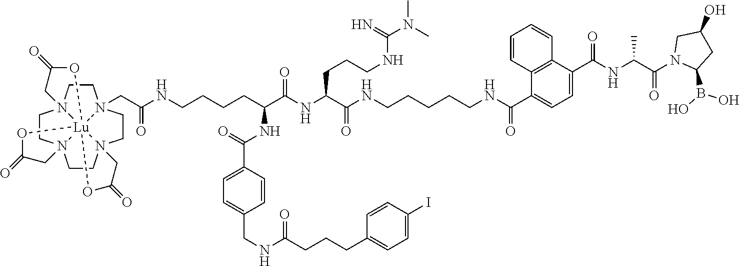 | 1761 (M + H) |
| | Lutetium(III) 2,2′,2″-(10-((10S,13S)-1-(4-(((R)-1-((2R,4S)-2-borono-4- | |
| | hydroxypyrrolidin-1-yl)-1-oxopropan-2-yl)carbamoyl)naphthalen-1-yl)-10- | |
| | (3-(3,3-dimethylguanidino)propyl)-13-(4-((4-(4- | |
| | iodophenyl)butanamido)methyl)benzamido)-1,9,12,19-tetraoxo-2,8,11,18- | |
| | tetraazaicosan-20-yl)-1,4,7,10-tetraazacyclododecane-1,4,7-triyl)triacetate | |
| 30 | 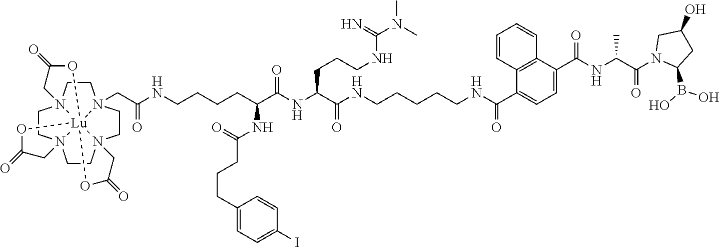 | 1628 (M + H) |
| | Lutetium(III) 2,2′,2″-(10-((10S,13S)-1-(4-(((R)-1-((2R,4S)-2-borono-4- | |
| | hydroxypyrrolidin-1-yl)-1-oxopropan-2-yl)carbamoyl)naphthalen-1-yl)-10- | |
| | (3-(3,3-dimethylguanidino)propyl)-13-(4-(4-iodophenyl)butanamido)- | |
| | 1,9,12,19-tetraoxo-2,8,11,18-tetraazaicosan-20-yl)-1,4,7,10- | |
| | tetraazacyclododecane-1,4,7-triyl)triacetate | |
| 31 | 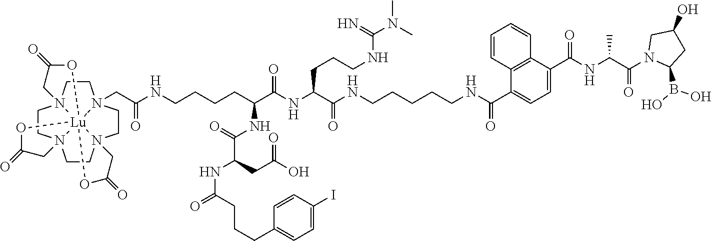 | 1725 (M − H₂O + H) |
| | Lutetium(III) 2,2′,2″-(10-((10S,13S)-1-(4-(((R)-1-((2R,4S)-2-borono-4- | |
| | hydroxypyrrolidin-1-yl)-1-oxopropan-2-yl)carbamoyl)naphthalen-1-yl)-13- | |
| | ((R)-3-carboxy-2-(4-(4-iodophenyl)butanamido)propanamido)-10-(3-(3,3- | |
| | dimethylguanidino)propyl)-1,9,12,19-tetraoxo-2,8,11,18-tetraazaicosan-20- | |
| | yl)-1,4,7,10-tetraazacyclododecane-1,4,7-triyl)triacetate | |
| 32 | 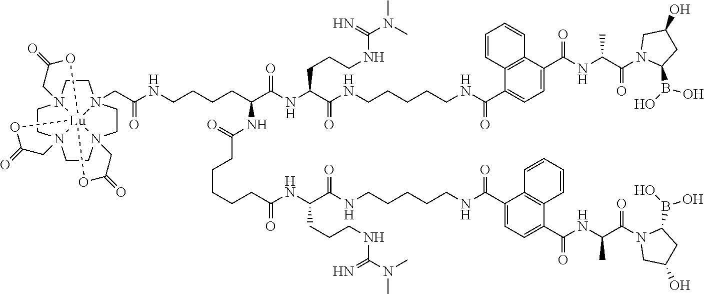 | 1075 (M/2 + H) |
| | Lutetium(III) 2,2′,2″-(10-((10S,20S)-1-(4-(((R)-1-((2R,4S)-2-borono-4- | |
| | hydroxypyrrolidin-1-yl)-1-oxopropan-2-yl)carbamoyl)naphthalen-1-yl)-20- | |
| | (((S)-17-(4-(((R)-1-((2R,4S)-2-borono-4-hydroxypyrrolidin-1-yl)-1- | |
| | oxopropan-2-yl)carbamoyl)naphthalen-1-yl)-3-imino-2-methyl-9,17-dioxo- | |
| | 2,4,10,16-tetraazaheptadecan-8-yl)carbamoyl)-10-(3-(3,3- | |
| | dimethylguanidino)propyl)-1,9,12,18,26-pentaoxo-2,8,11,19,25- | |
| | pentaazaheptacosan-27-yl)-1,4,7,10-tetraazacyclododecane-1,4,7- | |
| | triyl)triacetate | |
| 33 | 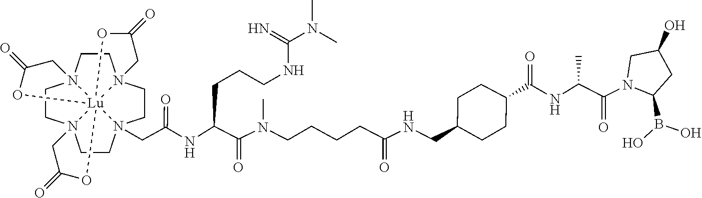 | 1198 (M + H) |
| | Lutetium(III) 2,2′,2″-(10-(2-(((S)-17-((1R,4S)-4-(((R)-1-((2R,4S)-2-borono- | |
| | 4-hydroxypyrrolidin-1-yl)-1-oxopropan-2-yl)carbamoyl)cyclohexyl)-3- | |
| | imino-2,10-dimethyl-9,15-dioxo-2,4,10,16-tetraazaheptadecan-8-yl)amino)- | |
| | 2-oxoethyl)-1,4,7,10-tetraazacyclodecane-1,4,7-triyl)triacetate | |
| 34 | 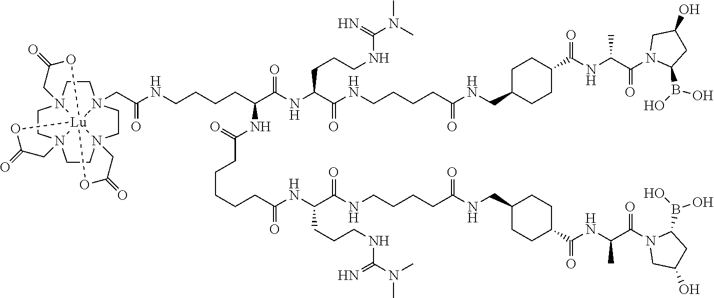 | 1031 (M/2 + H) |
| | Lutetium(III) 2,2′,2″-(10-((10R,20S)-1-((1R,4R)-4-(((R)-1-((2R,4S)-2- | |
| | borono-4-hydroxypyrrolidin-1-yl)-1-oxopropan-2-yl)carbamoyl)cyclohexyl)- | |
| | 20-(((S)-17-((1R,4S)-4-(((R)-1-((2R,4S)-2-borono-4-hydroxypyrrolidin-1- | |
| | yl)-1-oxopropan-2-yl)carbamoyl)cyclohexyl)-3-imino-2,10-dimethyl-9,15- | |
| | dioxo-2,4,10,16-tetraazaheptadecan-8-yl)carbamoyl)-10-(3-(3,3- | |
| | dimethylguanidino)propyl)-8-methyl-3,9,12,18,26-pentaoxo-2,8,11,19,25- | |
| | pentaazaheptacosan-27-yl)-1,4,7,10-tetraazacyclododecane-1,4,7- | |
| | triyl)triacetate | |
| 35 | 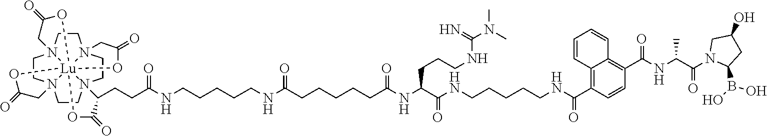 | 1508 (M − H₂O + H) |
| | Lutetium(III) 2,2′,2″-(10-((10S,29R)-1-(4-(((R)-1-((2R,4S)-2-borono-4- | |
| | hydroxypyrrolidin-1-yl)-1-oxopropan-2-yl)carbamoyl)naphthalen-1-yl)-29- | |
| | carboxy-10-(3-(3,3-dimethylguanidino)propyl)-1,9,12,18,26-pentaoxo- | |
| | 2,8,11,19,25-pentaazanonacosan-29-yl)-1,4,7,10-tetraazacyclododecane- | |
| | 1,4,7-triyl)triacetate | |
| 36 | 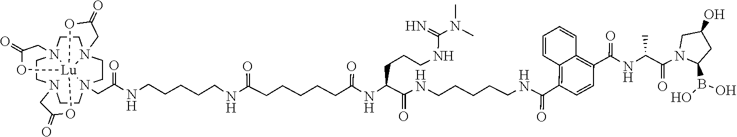 | 719 [(M − H₂O/2 + H] |
| | Lutetium(III) 2,2′,2″-(10-((S)-1-(4-(((R)-1-((2R,4S)-2-borono-4- | |
| | hydroxypyrrolidin-1-yl)-1-oxopropan-2-yl)carbamoyl)naphthalen-1-yl)-10- | |
| | (3-(3,3-dimethylguanidino)propyl)-1,9,12,18,26-pentaoxo-2,8,11,19,25- | |
| | pentaazaheptacosan-27-yl)-1,4,7,10-tetraazacyclododecane-1,4,7- | |
| | triyl)triacetate | |
| 37 | 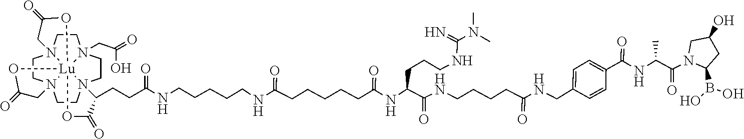 | 1458 (M − H₂O + H) |
| | Lutetium(III) 2,2′,2″-(10-((10S,29R)-1-(4-(((R)-1-((2R,4S)-2-borono-4- | |
| | hydroxypyrrolidin-1-yl)-1-oxopropan-2-yl)carbamoyl)phenyl)-29-carboxy- | |
| | 10-(3-(3,3-dimethylguanidino)propyl)-3,9,12,18,26-pentaoxo-2,8,11,19,25- | |
| | pentaazanonacosan-29-yl)-1,4,7,10-tetraazacyclododecane-1,4,7- | |
| | triyl)triacetate | |
| 38 | 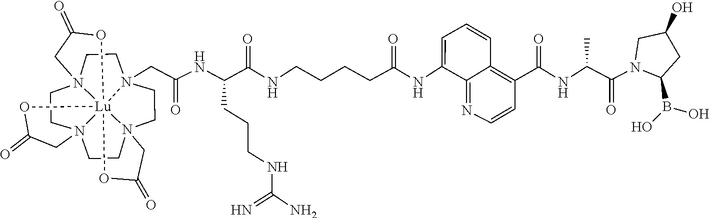 | 1186 (M + H) |
| | Lutetium(III) 2,2′,2″-(10-(2-(((S)-1-((5-((4-(((R)-1-((2R,4S)-2-borono-4- | |
| | hydroxypyrrolidin-1-yl)-1-oxopropan-2-yl)carbamoyl)quinolin-8-yl)amino)- | |
| | 5-oxopentyl)amino-5-guanidino-1-oxopentan-2-yl)amino)-2-oxoethyl)- | |
| | 1,4,7,10-tetraazacyclododecane-1,4,7-triyl)triacetate | |
| 39 | 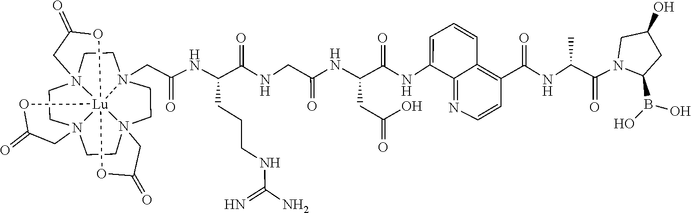 | 1259 (M + H) |
| | Lutetium(III) 2,2′,2″-(10-(2-(((S)-1-((2-(((S)-1-((4-(((R)-1-((2R,4S)-2- | |
| | borono-4-hydroxypyrrolidin-1-yl)-1-oxopropan-2-yl)carbamoyl)quinolin-8- | |
| | yl)amino)-3-carboxy-1-oxopropan-2-yl)amino)-2-oxoethyl)amino)-5- | |
| | guanidino-1-oxopentan-2-yl)amino)-2-oxoethyl)-1,4,7,10- | |
| | tetraazacyclododecane-1,4,7-triyl)triacetate | |
| 49 | 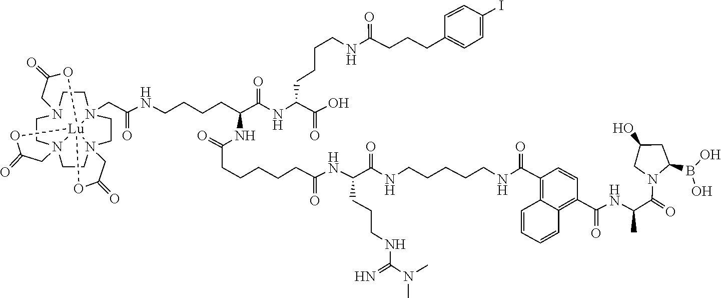 | 1880 (M + H) |
| | Lutetium(III) 2,2′,2″-(10-((10S,20S)-1-(4-(((R)-1-((2R,4S)-2-borono-4- | |
| | hydroxypyrrolidin-1-yl)-1-oxopropan-2-yl)carbamoyl)naphthalen-1-yl)-20- | |
| | (((R)-1-carboxy-5-(4-(4-iodophenyl)butanamido)pentyl)carbamoyl)-10-(3- | |
| | (3,3-dimethylguanidino)propyl)-1,9,12,18,26-pentaoxo-2,8,11,19,25- | |
| | pentaazaheptacosan-27-yl)-1,4,7,10-tetraazacyclododecane-1,4,7- | |
| | triyl)triacetic acid | |

### Example 40

### Radiolabeling and Formulation of Compounds

### 177-Lutetium(III) 2,2′,2″-(10-(2-(((S)-1-((5-((4-(((R)-1-((2R,4S)-2-borono-4-hydroxypyrrolidin-1-yl)-1-oxopropan-2-yl)carbamoyl)benzyl)amino)-5-oxopentyl)amino)-5-guanidino-1-oxopentan-2-yl)amino)-2-oxoethyl)-1,4,7,10-tetraazacyclododecane-1,4,7-triyl)triacetate

2,2′,2″-(10-(2-(((S)-1-((5-((4-(((R)-1-((2R,4S)-2-Borono-4-hydroxypyrrolidin-1-yl)-1-oxopropan-2-yl)carbamoyl)benzyl)amino)-5-oxopentyl)amino)-5-guanidino-1-oxopentan-2-yl)amino)-2-oxoethyl)-1,4,7,10-tetraazacyclododecane-1,4,7-triyl)triacetic acid (10.4 μg) was radiolabeled with 903 MBq of ¹⁷⁷Lu[LuCl₃] in an aqueous mixture of 0.4 M N-acetyl L-methionine (NAM), 0.2 M sodium acetate, gentisic acid (5 mg/mL) and sodium ascorbate (10 mg/mL), pH 4. The reaction was heated to 70° C. for 20 min with shaking (500 rpm) before being quenched with 650 mL of formulation buffer containing sodium ascorbate (5.85 mg/mL), NAM (8.7 mg/mL), and diethylenetriaminepentaacetic acid (DTPA, 0.118 mg/mL) and passed through a 0.22 μm filter. The final formulated product included 0.1 M NAM, 0.06 M sodium acetate, gentisic acid (0.77 mg/mL), sodium ascorbate (6.5 mg/mL), and diethylenetriaminepentaacetic acid (DTPA, 0.1 mg/mL). The radiochemical purity (RCP) at the end of synthesis was 93.5% by radioHPLC (Waters XBridge® BEH C18 column (3.5 μm, 4.6×150 mm)) and mobile phase of A (0.5% TFA in Water) and B (0.5% TFA in ACN) in the gradient (flow rate 1.1 mL/min): 0-5 min 95% A, 5-23 min 95-66.5% A, 23-24 min 66.5-0% A, 24-27 min 0% A, 27-28 min 0-95% A, 28-33 min 95% A.

### Example 50

### 177-Lutetium(III) 2,2′,2″-(10-((4S,10S)-1-(4-(((R)-1-((2R,4S)-2-borono-4-hydroxypyrrolidin-1-yl)-1-oxopropan-2-yl)carbamoyl)phenyl)-4-(carboxymethyl)-10-(3-(3,3-dimethylguanidino)propyl)-3,6,9,12-tetraoxo-2,5,8,11-tetraazatridecan-13-yl)-1,4,7,10-tetraazacyclododecane-1,4,7-triyl)triacetate

2,2′,2″-(10-((4S,10S)-1-(4-(((R)-1-((2R,4S)-2-Borono-4-hydroxypyrrolidin-1-yl)-1-oxopropan-2-yl)carbamoyl)phenyl)-4-(carboxymethyl)-10-(3-(3,3-dimethylguanidino)propyl)-3,6,9,12-tetraoxo-2,5,8,11-tetraazatridecan-13-yl)-1,4,7,10-tetraazacyclododecane-1,4,7-triyl)triacetic acid (7 μg) was radiolabeled with 1000 MBq of 177Lu[LuCl₃] in an aqueous mixture of 0.4 M NAM, 0.2 M sodium acetate, gentisic acid (5 mg/mL) and sodium ascorbate (10 mg/mL), pH 4.5. The reaction was heated to 70° C. for 20 min with shaking (500 rpm) before being quenched with 704 mL of formulation buffer containing sodium ascorbate (5.85 mg/mL), NAM (8.7 mg/mL) and DTPA (0.118 mg/mL) and passed through a 0.22 μm filter. The final formulated product included 0.1 M NAM, 0.06 M sodium acetate, gentisic acid (0.77 mg/mL), sodium ascorbate (6.5 mg/mL) and DTPA (0.1 mg/mL). The radiochemical purity (RCP) at the end of synthesis was 92.5% by radioHPLC (Waters XBridge® BEH C18 column (3.5 μm, 4.6×150 mm)) and mobile phase of A (0.5% TFA in Water) and B (0.5% TFA in ACN) in the gradient (flow rate 1.1 mL/min): 0-10 min 95% A, 10-28 min 95-66.5% A, 28-29 min 66.5-0% A, 29-32 min 0% A, 32-33 min 0-95% A, 33-38 min 95% A.

### Example 51

### Lutetium(III) 2,2′,2″-(10-(2-(((S)-1-((5-((4-((2-((2R,4S)-2-borono-4-hydroxypyrrolidin-1-yl)-2-oxoethyl)carbamoyl)benzyl)(methyl)amino)-5-oxopentyl)amino)-5-guanidino-1-oxopentan-2-yl)amino)-2-oxoethyl)-1,4,7,10-tetraazacyclododecane-1,4,7-triyl)triacetate

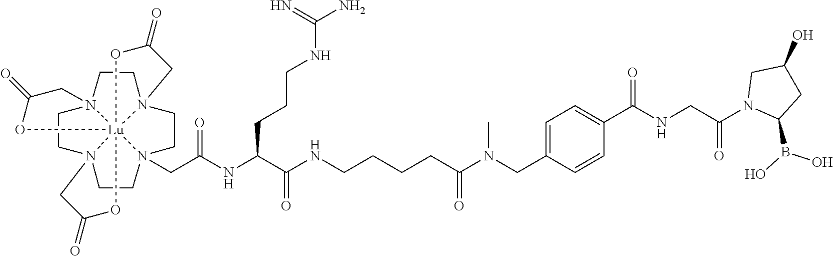

2,2′,2″-(10-(2-(((S)-1-((5-((4-((2-((2R,4S)-2-Borono-4-hydroxypyrrolidin-1-yl)-2-oxoethyl)carbamoyl)benzyl)(methyl)amino)-5-oxopentyl)amino)-5-guanidino-1-oxopentan-2-yl)amino)-2-oxoethyl)-1,4,7,10-tetraazacyclododecane-1,4,7-triyl)triacetic acid was reacted with LuCl₃ according to SP-8 to give the title compound. ESI/MS m/z: 1150 (M+H)

The following compounds in Table 26 were synthesized in essentially the same manner as described for lutetium(III) 2,2′,2″-(10-(2-(((S)-1-((5-((4-((2-((2R,4S)-2-borono-4-hydroxypyrrolidin-1-yl)-2-oxoethyl)carbamoyl)benzyl)(methyl)amino)-5-oxopentyl)amino)-5-guanidino-1-oxopentan-2-yl)amino)-2-oxoethyl)-1,4,7,10-tetraazacyclododecane-1,4,7-triyl)triacetate.

TABLE 26

| Ex | | |
|---|---|---|
| # | Structure | ESI/MS |
| 52 | 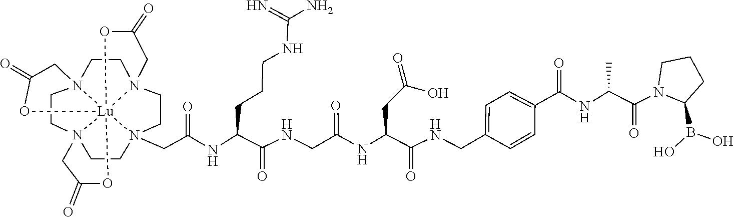 | 1189 (M − H₂O + H) |
| | Lutetium(III) 2,2′,2″-(10-((4S,10S)-1-(4-(((R)-1-((R)-2-boronopyrrolidin-1- | |
| | yl)-1-oxopropan-2-yl)carbamoyl)phenyl)-4-(carboxymethyl)-10-(3- | |
| | guanidinopropyl)-3,6,9,12-tetraoxo-2,5,8,11-tetraazatridecan-13-yl)- | |
| | 1,4,7,10-tetraazacyclododecane-1,4,7-triyl)triacetate | |
| 53 | 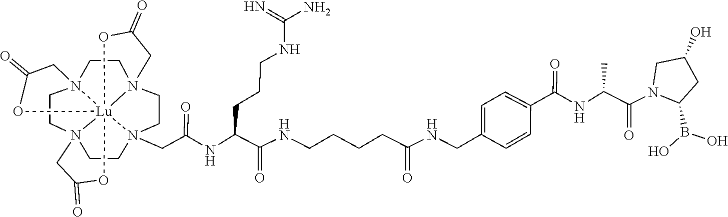 | 1149 (M + H) |
| | Lutetium(III) 2,2′,2″-(10-(2-(((S)-1-((5-((4-(((R)-1-((2R,4R)-2-borono-4- | |
| | hydroxypyrrolidin-1-yl)-1-oxopropan-2-yl)carbamoyl)benzyl)amino)-5- | |
| | oxopentyl)amino)-5-guanidino-1-oxopentan-2-yl)amino)-2-oxoethyl)- | |
| | 1,4,7,10-tetraazacyclododecane-1,4,7-triyl)triacetate | |
| 55 | 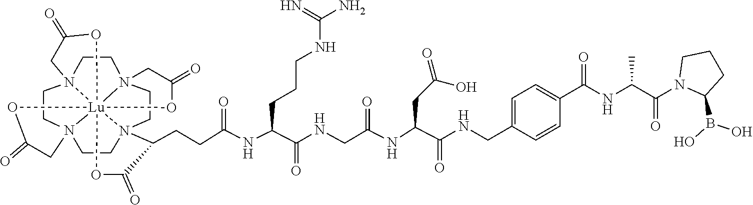 | 1260 (M − H₂O + H) |
| | Lutetium(III) 2,2′,2″-(10-((4S,10S,15R)-1-(4-(((R)-1-((R)-2- | |
| | boronopyrrolidin-1-yl)-1-oxopropan-2-yl)carbamoyl)phenyl)-15-carboxy-4- | |
| | (carboxymethyl)-10-(3-guanidinopropyl)-3,6,9,12-tetraoxo-2,5,8,11- | |
| | tetraazapentadecan-15-yl)-1,4,7,10-tetraazacyclododecane-1,4,7- | |
| | triyl)triacetate | |
| 56 | 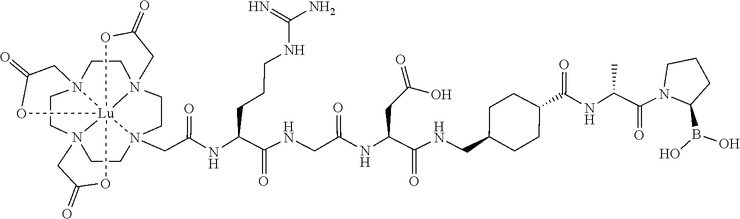 | 1195 (M + H) |
| | Lutetium(III) 2,2′,2″-(10-((4S,10S)-1-((1R,4S)-4-(((R)-1-((R)-2- | |
| | boronopyrrolidin-1-yl)-1-oxopropan-2-yl)carbamoyl)cyclohexyl)-4- | |
| | (carboxymethyl)-10-(3-guanidinopropyl)-3,6,9,12-tetraoxo-2,5,8,11- | |
| | tetraazatridecan-13-yl)-1,4,7,10-tetraazacyclododecane-1,4,7-triyl)triacetate | |
| 57 | 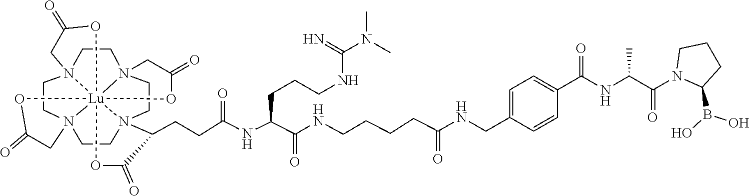 | 1216 (M − H₂O + H) |
| | Lutetium(III) 2,2′,2″-(10-((R)-4-(((S)-17-(4-(((R)-1-((R)-2-boronopyrrolidin- | |
| | 1-yl)-1-oxopropan-2-yl)carbamoyl)phenyl)-3-imino-2-methyl-9,15-dioxo- | |
| | 2,4,10,16-tetraazaheptadecan-8-yl)amino)-1-carboxy-4-oxobutyl)-1,4,7,10- | |
| | tetraazacyclododecane-1,4,7-triyl)triacetate | |
| 58 | 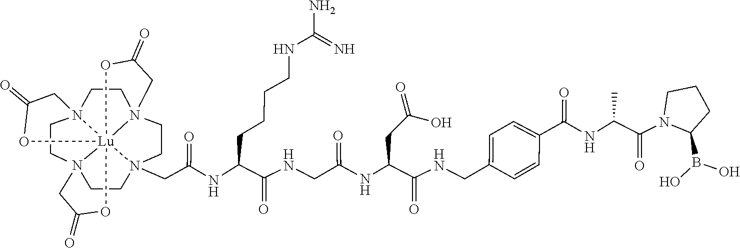 | 1203 (M − H₂O + H) |
| | Lutetium(III) 2,2′,2″-(10-((4S,10S)-1-(4-(((R)-1-((R)-2-boronopyrrolidin-1- | |
| | yl)-1-oxopropan-2-yl)carbamoyl)phenyl)-4-(carboxymethyl)-10-(4- | |
| | guanidinobutyl)-3,6,9,12-tetraoxo-2,5,8,11-tetraazatridecan-13-yl)-1,4,7,10- | |
| | tetraazacyclododecane-1,4,7-triyl)triacetate | |
| 59 | 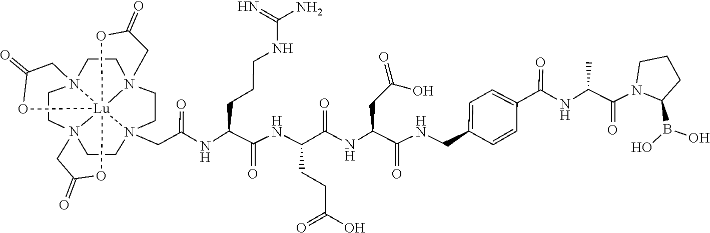 | 1261 (M − H₂O + H) |
| | Lutetium(III) 2,2′,2″-(10-((4S,7S,10S)-1-(4-(((R)-1-((R)-2-boronopyrrolidin- | |
| | 1-yl)-1-oxopropan-2-yl)carbamoyl)phenyl)-7-(2-carboxyethyl)-4- | |
| | (carboxymethyl)-10-(3-guanidinopropyl)-3,6,9,12-tetraoxo-2,5,8,11- | |
| | tetraazatridecan-13-yl)-1,4,7,10-tetraazacyclododecane-1,4,7-triyl)triacetate | |
| 60 | 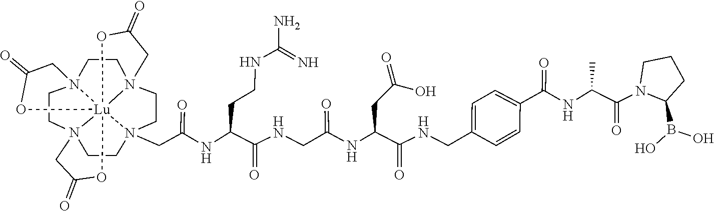 | 1192 (M + H) |
| | Lutetium(III) 2,2′,2″-(10-((4S,10S)-1-(4-(((R)-1-((R)-2-boronopyrrolidin-1- | |
| | yl)-1-oxopropan-2-yl)carbamoyl)phenyl)-4-(carboxymethyl)-10-(2- | |
| | guanidinoethyl)-3,6,9,12-tetraoxo-2,5,8,11-tetraazatridecan-13-yl)-1,4,7,10- | |
| | tetraazacyclododecane-1,4,7-triyl)triacetate | |
| 61 | 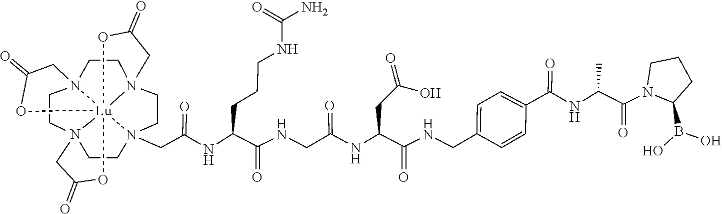 | 1190 (M − H₂O + H) |
| | Lutetium(III) 2,2′,2″-(10-((4S,10S)-1-(4-(((R)-1-((R)-2-boronopyrrolidin-1- | |
| | yl)-1-oxopropan-2-yl)carbamoyl)phenyl)-4-(carboxymethyl)-3,6,9,12- | |
| | tetraoxo-10-(3-ureidopropyl)-2,5,8,11-tetraazatridecan-13-yl)-1,4,7,10- | |
| | tetraazacyclododecane-1,4,7-triyl)triacetate | |
| 62 | 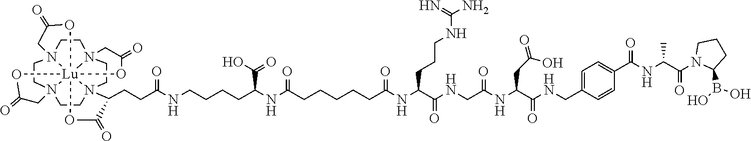 | 1531 (M − H₂O + H) |
| | Lutetium(III) (2S,8S,18S,27R)-2-(((R)-1-((R)-2-boronopyrrolidin-1-yl)- | |
| | 1-oxopropan-2-yl)carbamoyl)benzyl)carbamoyl)-8-(3-guanidinopropyl)- | |
| | 4,7,10,16,24-pentaoxo-27-(4,7,10-tris(carboxymethyl)-1,4,7,10- | |
| | tetraazacyclododecan-1-yl)-3,6,9,17,23-pentaazaheptacosane-1,18,27- | |
| | tricarboxylate | |
| 63 | 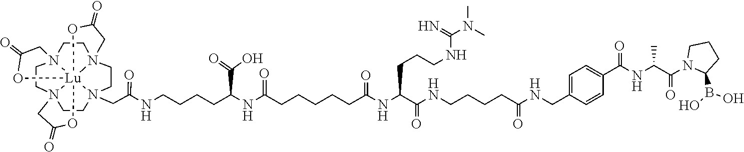 | 1414 (M − H₂O + H) |
| | Lutetium(III) 2,2′,2″-(10-((10S,20S)-1-(4-(((R)-1-((R)-2-boronopyrrolidin-1- | |
| | yl)-1-oxopropan-2-yl)carbamoyl)phenyl)-20-carboxy-10-(3-(3,3- | |
| | dimethylguanidino)propyl)-3,9,12,18,26-pentaoxo-2,8,11,19,25- | |
| | pentaazaheptacosan-27-yl)-1,4,7,10-tetraazacyclododecane-1,4,7- | |
| | triyl)triacetate | |
| 64 | 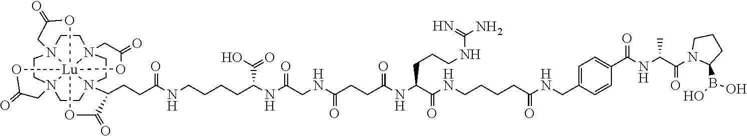 | 1473 (M − H₂O + H) |
| | Lutetium(III) 2,2′,2″-(10-((10S,20R,29R)-1-(4-(((R)-1-((R)-2- | |
| | boronopyrrolidin-1-yl)-1-oxopropan-2-yl)carbamoyl)phenyl)-20,29- | |
| | dicarboxy-10-(3-guanidinopropyl)-3,9,12,15,18,26-hexaoxo-2,8,11,16,19,25- | |
| | hexaazanonacosan-29-yl)-1,4,7,10-tetraazacyclododecane-1,4,7- | |
| | triyl)triacetate | |
| 65 | 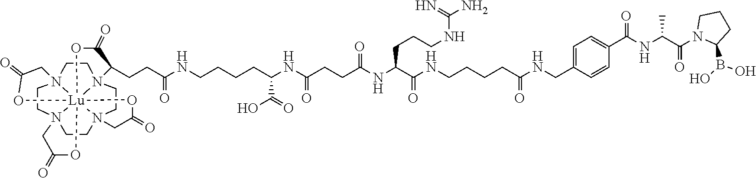 | 1416 (M − H₂O + H) |
| | Lutetium(III) 2,2′,2″-(10-((10S,17S,26R)-1-(4-(((R)-1-((R)-2- | |
| | boronopyrrolidin-1-yl)-1-oxopropan-2-yl)carbamoyl)phenyl)-17,26- | |
| | dicarboxy-10-(3-guanidinopropyl)-3,9,12,15,23-pentaoxo-2,8,11,16,22- | |
| | pentaazahexacosan-26-yl)-1,4,7,10-tetraazacyclododecane-1,4,7- | |
| | triyl)triacetate | |
| 66 | 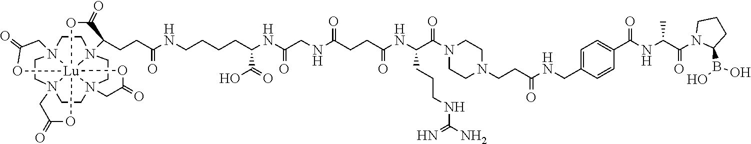 | 1514 (M − H₂O + H) |
| | Lutetium(III) 2,2′,2″-(10-((6S,16S,25R)-1-amino-6-(4-(3-((4-(((R)-1-((R)-2- | |
| | boronopyrrolidin-1-yl)-1-oxopropan-2-yl)carbamoyl)benzyl)amino)-3- | |
| | oxopropyl)piperazine-1-carbonyl)-16,25-dicarboxy-1-imino-8,11,14,22- | |
| | tetraoxo-2,7,12,15,21-pentazaapentacosan-25-yl)-1,4,7,10- | |
| | tetraazacyclododecane-1,4,7-triyl)triacetate | |
| 67 | 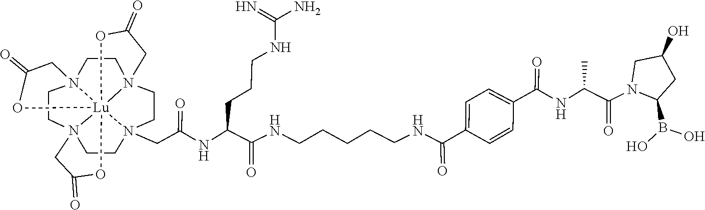 | 1150 (M + H) |
| | Lutetium(III) 2,2′,2″-(10-(2-(((S)-1-((5-(4-(((R)-1-((2R,4S)-2-borono-4- | |
| | hydroxypyrrolidin-1-yl)-1-oxopropan-2- | |
| | yl)carbamoyl)benzamido)pentyl)amino)-5-guanidino-1-oxopentan-2- | |
| | yl)amino)-2-oxoethyl)-1,4,7,10-tetraazacyclododecane-1,4,7-triyl)triacetate | |
| 68 | 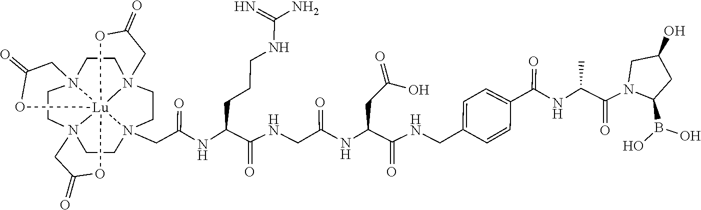 | 1223 (M + H) |
| | Lutetium(III) 2,2′,2″-(10-((4S,10S)-1-(4-(((R)-1-((2R,4S)-2-borono-4- | |
| | hydroxypyrrolidin-1-yl)-1-oxopropan-2-yl)carbamoyl)phenyl)-4- | |
| | (carboxymethyl)-10-(3-guanidinopropyl)-3,6,9,12-tetraoxo-2,5,8,11- | |
| | tetraazatridecan-13-yl)-1,4,7,10-tetraazacyclododecane-1,4,7-triyl)triacetate | |
| 69 | 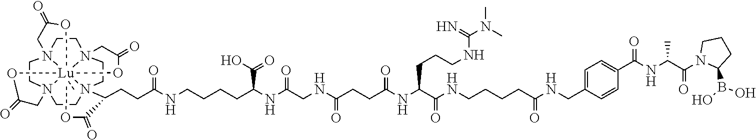 | |
| | Lutetium(III) 2,2′,2″-(10-((10S,20S,29R)-1-(4-(((R)-1-((R)-2- | |
| | boronopyrrolidin-1-yl)-1-oxopropan-2-yl)carbamoyl)phenyl)-20,29- | |
| | dicarboxy-10-(3-(3,3-dimethylguanidino)propyl)-3,9,12,15,18,26-hexaoxo- | |
| | 2,8,11,16,19,25-hexaazanonacosan-29-yl)-1,4,7,10-tetraazacyclododecane- | |
| | 1,4,7-triyl)triacetate | |
| 70 | 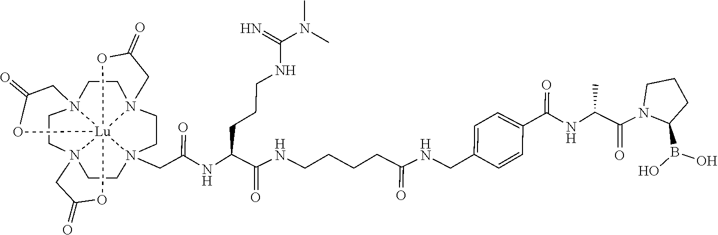 | 1162 (M + H) |
| | Lutetium(III) 2,2′,2″-(10-(2-(((S)-17-(4-(((R)-1-((R)-2-boronopyrrolidin-1- | |
| | yl)-1-oxopropan-2-yl)carbamoyl)phenyl)-3-imino-2-methyl-9,15-dioxo- | |
| | 2,4,10,16-tetraazaheptadecan-8-yl)amino)-2-oxoethyl)-1,4,7,10- | |
| | tetraazacyclododecane-1,4,7-triyl)triacetate | |
| 71 | 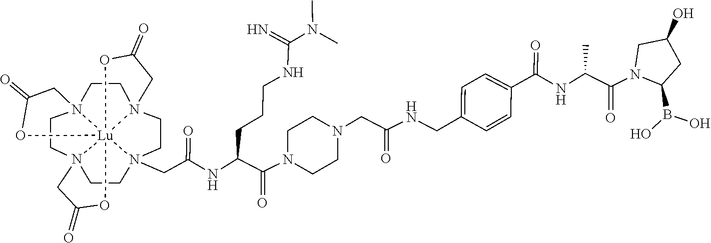 | 1205 (M + H) |
| | Lutetium(III) 2,2′,2″-(10-(2-(((S)-1-(4-(2-((4-(((R)-1-((2R,4S)-2-borono-4- | |
| | hydroxypyrrolidin-1-yl)-1-oxopropan-2-yl)carbamoyl)benzyl)amino)-2- | |
| | oxoethyl)piperazin-1-yl)-5-(3,3-dimethylguanidino)-1-oxopentan-2- | |
| | yl)amino)-2-oxoethyl)-1,4,7,10-tetraazacyclododecane-1,4,7-triyl)triacetate | |
| 72 | 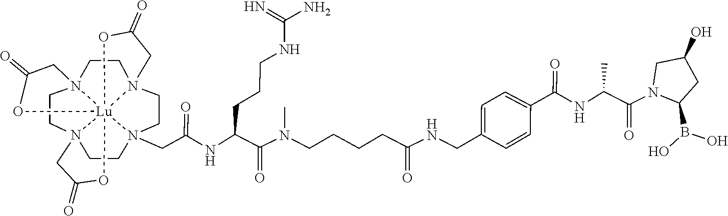 | 1164 (M + H) |
| | Lutetium(III) 2,2′,2″-(10-(2-(((S)-1-((5-((4-(((R)-1-((2R,4S)-2-borono-4- | |
| | hydroxypyrrolidin-1-yl)-1-oxopropan-2-yl)carbamoyl)benzyl)amino)-5- | |
| | oxopentyl)(methyl)amino)-5-guanidino-1-oxopentan-2-yl)amino)-2- | |
| | oxoethyl)-1,4,7,10-tetraazacyclododecane-1,4,7-triyl)triacetate | |
| 73 | 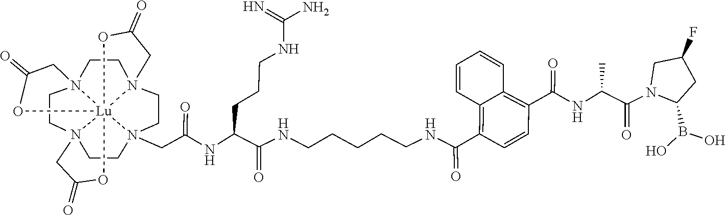 | 1202 (M + H) |
| | Lutetium(III) 2,2′,2″-(10-(2-(((S)-1-((5-(4-(((R)-1-((2S,4S)-2-borono-4- | |
| | fluoropyrrolidin-1-yl)-1-oxopropan-2-yl)carbamoyl)-1- | |
| | naphthamido)pentyl)amino)-5-guanidino-1-oxopentan-2-yl)amino)-2- | |
| | oxoethyl)-1,4,7,10-tetraazacyclododecane-1,4,7-triyl)triacetate | |
| 74 | 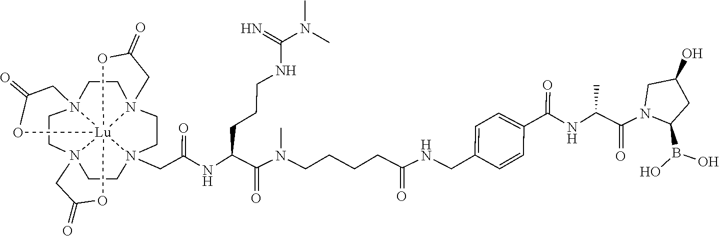 | 1192 (M + H) |
| | Lutetium(III) 2,2′,2″-(10-(2-(((S)-17-(4-(((R)-1-((2R,4S)-2-borono-4- | |
| | hydroxypyrrolidin-1-yl)-1-oxopropan-2-yl)carbamoyl)phenyl)-3-imino-2,10- | |
| | dimethyl-9,15-dioxo-2,4,10,16-tetraazaheptadecan-8-yl)amino)-2-oxoethyl)- | |
| | 1,4,7,10-tetraazacyclododecane-1,4,7-triyl)triacetate | |
| 75 | 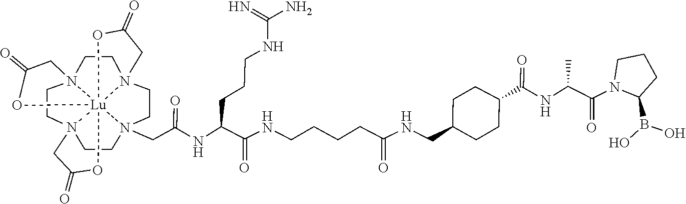 | 1140 (M + H) |
| | Lutetium(III) 2,2′,2″-(10-(2-(((S)-1-((5-((((1R,4S)-4-(((R)-1-((R)-2- | |
| | boronopyrrolidin-1-yl)-1-oxopropan-2- | |
| | yl)carbamoyl)cyclohexyl)methyl)amino)-5-oxopentyl)amino)-5-guanidino- | |
| | 1-oxopentan-2-yl)amino)-2-oxoethyl)-1,4,7,10-tetraazacyclododecane-1,4,7- | |
| | triyl)triacetate | |
| 76 | 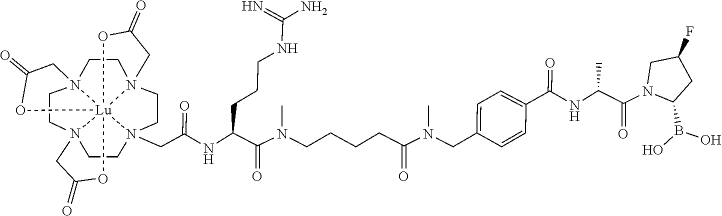 | 1180 (M + H) |
| | Lutetium(III) 2,2′,2″-(10-(2-(((S)-1-((5-((4-(((R)-1-((2S,4S)-2-borono-4- | |
| | fluoropyrrolidin-1-yl)-1-oxopropan-2-yl)carbamoyl)benzyl)(methyl)amino)- | |
| | 5-oxopentyl)(methyl)amino)-5-guanidino-1-oxopentan-2-yl)amino)-2- | |
| | oxoethyl)-1,4,7,10-tetraazacyclododecane-1,4,7-triyl)triacetate | |
| 77 | 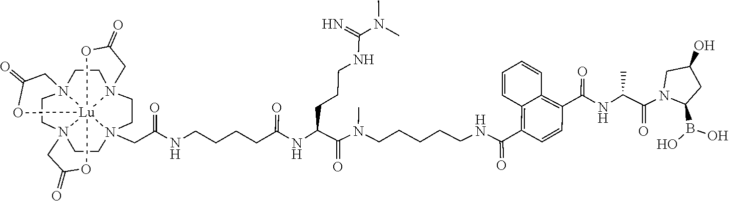 | 1341 (M + H) |
| | Lutetium(III) 2,2′,2″-(10-((S)-1-(4-(((R)-1-((2R,4S)-2-borono-4- | |
| | hydroxypyrrolidin-1-yl)-1-oxopropan-2-yl)carbamoyl)naphthalen-1-yl)-10- | |
| | (3-(3,3-dimethylguanidino)propyl)-8-methyl-1,9,12,18-tetraoxo-2,8,11,17- | |
| | tetraazanonadecan-19-yl)-1,4,7,10-tetraazacyclododecane-1,4,7- | |
| | triyl)triacetate | |
| 78 | 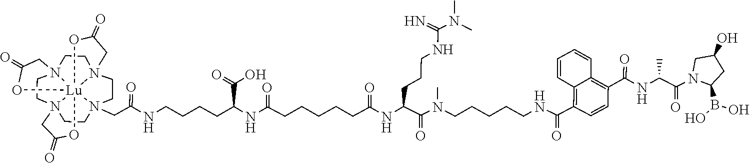 | 748 (M − H₂O/2 + H) |
| | Lutetium(III) 2,2′,2″-(10-((10S,20S)-1-(4-(((R)-1-((2R,4S)-2-borono-4- | |
| | hydroxypyrrolidin-1-yl)-1-oxopropan-2-yl)carbamoyl)naphthalen-1-yl)-20- | |
| | carboxy-10-(3-(3,3-dimethylguanidino)propyl)-8-methyl-1,9,12,18,26- | |
| | pentaoxo-2,8,11,19,25-pentaazaheptacosan-27-yl)-1,4,7,10- | |
| | tetraazacyclododecane-1,4,7-triyl)triacetate | |
| 79 | 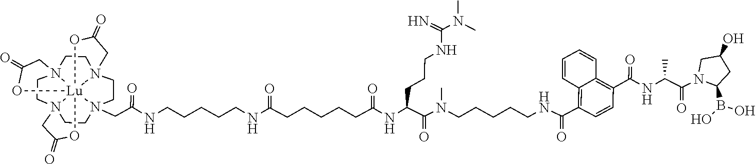 | 1468 (M + H) |
| | Lutetium(III) 2,2′,2″-(10-((S)-1-(4-(((R)-1-((2R,4S)-2-borono-4- | |
| | hydroxypyrrolidin-1-yl)-1-oxopropan-2-yl)carbamoyl)naphthalen-1-yl)-10- | |
| | (3-(3,3-dimethylguanidino)propyl)-8-methyl-1,9,12,18,26-pentaoxo- | |
| | 2,8,11,19,25-pentaazaheptacosan-27-yl)-1,4,7,10-tetraazacyclododecane- | |
| | 1,4,7-triyl)triacetate | |
| 80 | 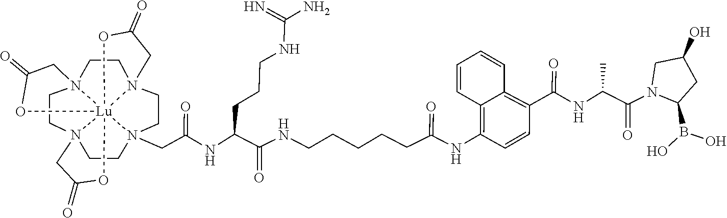 | 1201 (M + H) |
| | Lutetium(III) 2,2′,2″-(10-(2-(((S)-1-((6-((4-(((R)-1-((2R,4S)-2-borono-4- | |
| | hydroxypyrrolidin-1-yl)-1-oxopropan-2-yl)carbamoyl)naphthalen-1- | |
| | yl)amino)-6-oxohexyl)amino)-5-guanidino-1-oxopentan-2-yl)amino)-2- | |
| | oxoethyl)-1,4,7,10-tetraazacyclododecane-1,4,7-triyl)triacetate | |
| 81 | 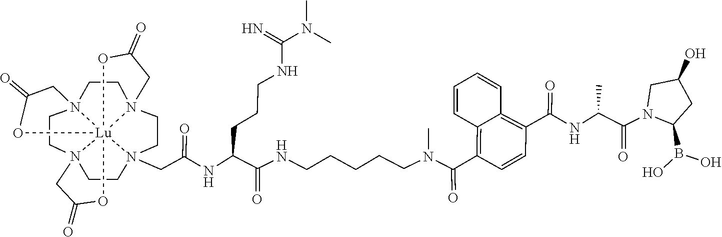 | 1242 (M + H) |
| | Lutetium(III) 2,2′,2″-(10-(2-(((S)-17-(4-(((R)-1-((2R,4S)-2-borono-4- | |
| | hydroxypyrrolidin-1-yl)-1-oxopropan-2-yl)carbamoyl)naphthalen-1-yl)-3- | |
| | imino-2,16-dimethyl-9,17-dioxo-2,4,10,16-tetraazaheptadecan-8-yl)amino)- | |
| | 2-oxoethyl)-1,4,7,10-tetraazacyclododecane-1,4,7-triyl)triacetate | |
| 82 | 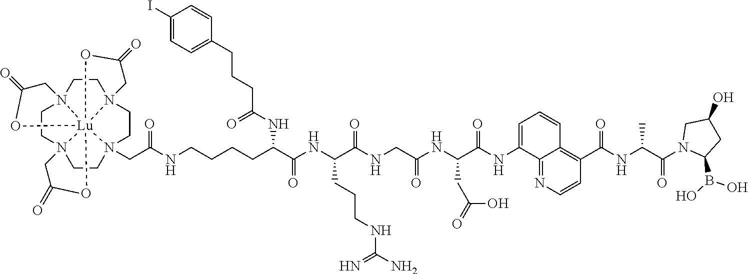 | 1642 (M − H₂O + H) |
| | Lutetium(III) 2,2′,2″-(10-((2S,8S,11S)-1-((4-(((R)-1-((2R,4S)-2-borono-4- | |
| | hydroxypyrrolidin-1-yl)-1-oxopropan-2-yl)carbamoyl)quinolin-8-yl)amino)- | |
| | 2-(carboxymethyl)-8-(3-guanidinopropyl)-11-(4-(4-iodophenyl)butanamido)- | |
| | 1,4,7,10,17-pentaoxo-3,6,9,16-tetraazaoctadecan-18-yl)-1,4,7,10- | |
| | tetraazacyclododecane-1,4,7-triyl)triacetate | |
| 83 | 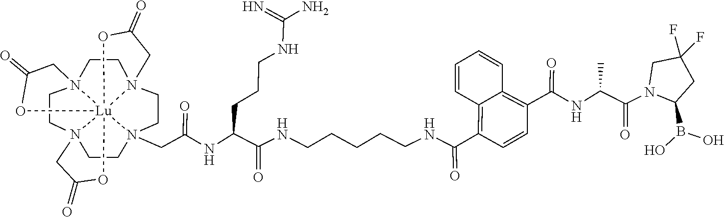 | 1219 (M + H) |
| | Lutetium(III) 2,2′,2″-(10-(2-(((S)-1-((5-(4-(((R)-1-((R)-2-borono-4,4- | |
| | difluoropyrrolidin-1-yl)-1-oxopropan-2-yl)carbamoyl)-1- | |
| | naphthamido)pentyl)amino)-5-guanidino-1-oxopentan-2-yl)amino)-2- | |
| | oxoethyl)-1,4,7,10-tetraazacyclododecane-1,4,7-triyl)triacetate | |

The following compounds in Table 27 were synthesized in essentially the same manner as SP-8 except using GaCl₃ instead of LuCl₃.

TABLE 27

| | | ESI/ |
|---|---|---|
| Ex # | Structure | MS |
| 84 | 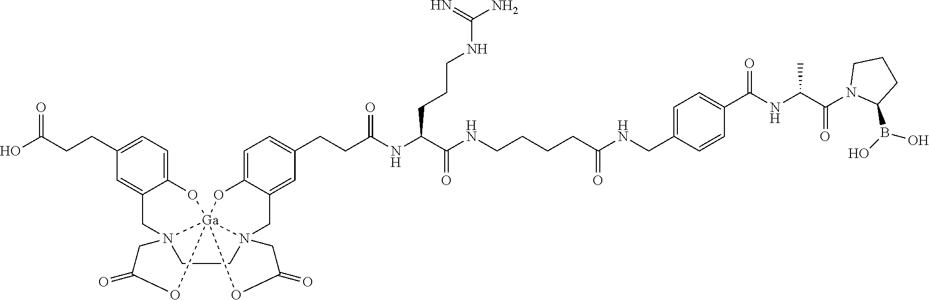 | 1137 (M − H₂O + H) |
| | Gallium(III) N-(5-(3-(((S)-1-((5-((4-(((R)-1-((R)-2-boronopyrrolidin-1-yl)-1- | |
| | oxopropan-2-yl)carbamoyl)benzyl)amino)-5-oxopentyl)amino)-5-guanidino- | |
| | 1-oxopentan-2-yl)amino)-3-oxopropyl)-2-oxidobenzyl)-N-(2-((5-(2- | |
| | carboxyethyl)-2-oxidobenzyl)(carboxylatomethyl)amino)ethyl)glycinate | |
| 85 | 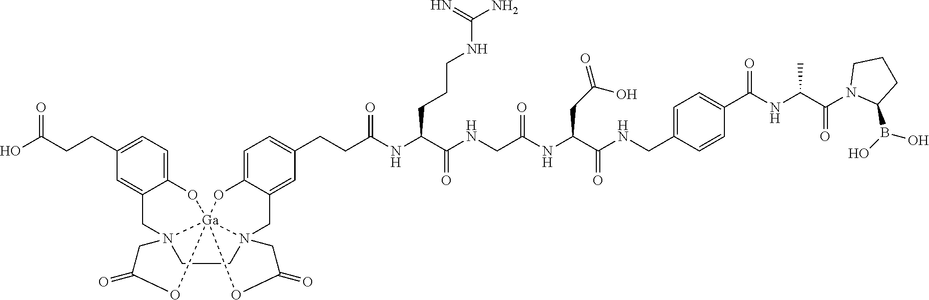 | 1210 (M − H₂O + H) |
| | Gallium(III) N-(5-((4S,10S)-1-(4-(((R)-1-((R)-2-boronopyrrolidin-1-yl)-1- | |
| | oxopropan-2-yl)carbamoyl)phenyl)-4-(carboxymethyl)-10-(3- | |
| | guanidinopropyl)-3,6,9,12-tetraoxo-2,5,8,11-tetraazatetradecan-14-yl)-2- | |
| | oxidobenzyl)-N-(2-((5-(2-carboxyethyl)-2- | |
| | oxidobenzyl)(carboxylatomethyl)amino)ethyl)glycinate | |
| 86 | 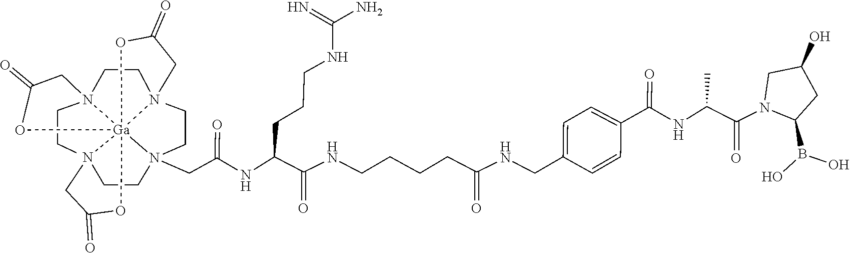 | 1044 (M + H) |
| | Gallium(III) 2,2′,2″-(10-(2-(((S)-1-((5-((4-(((R)-1-((2R,4S)-2-borono-4- | |
| | hydroxypyrrolidin-1-yl)-1-oxopropan-2-yl)carbamoyl)benzyl)amino)-5- | |
| | oxopentyl)amino)-5-guanidino-1-oxopentan-2-yl)amino)-2-oxoethyl)- | |
| | 1,4,7,10-tetraazacyclododecane-1,4,7-triyl)triacetate | |
| 87 | 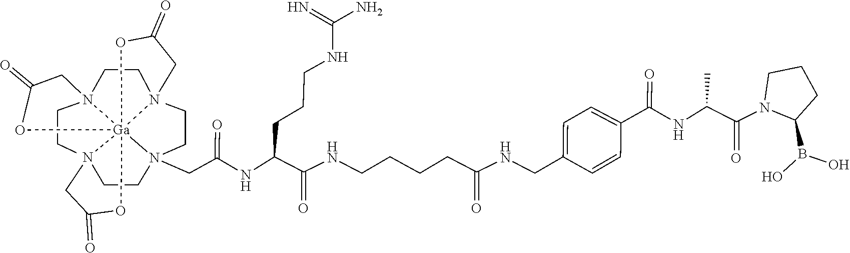 | 1027 (M + H) |
| | Gallium(III) 2,2′,2″-(10-(2-(((S)-1-((5-((4-(((R)-1-((R)-2-boronopyrrolidin-1- | |
| | yl)-1-oxopropan-2-yl)carbamoyl)benzyl)amino)-5-oxopentyl)amino)-5- | |
| | guanidino-1-oxopentan-2-yl)amino)-2-oxoethyl)-1,4,7,10- | |
| | tetraazacyclododecane-1,4,7-triyl)triacetate | |
| 88 | 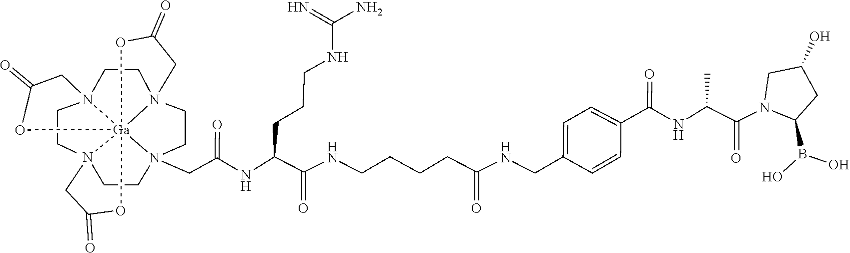 | 1044 (M + H) |
| | Gallium(III) 2,2′,2″-(10-(2-(((S)-1-((5-((4-(((R)-1-((2R,4R)-2-borono-4- | |
| | hydroxypyrrolidin-1-yl)-1-oxopropan-2-yl)carbamoyl)benzyl)amino)-5- | |
| | oxopentyl)amino)-5-guanidino-1-oxopentan-2-yl)amino)-2-oxoethyl)- | |
| | 1,4,7,10-tetraazacyclododecane-1,4,7-triyl)triacetate | |
| 89 | 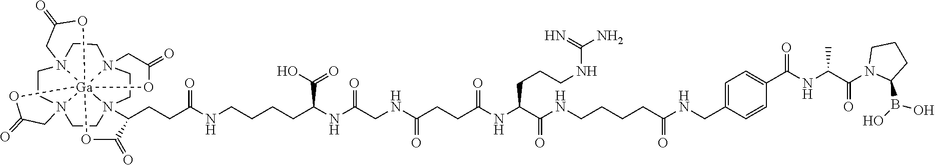 | 1367 (M − H₂O + H) |
| | Gallium(III) 2,2′,2″-(10-((10S,20S,29R)-1-(4-(((R)-1-((R)-2- | |
| | boronopyrrolidin-1-yl)-1-oxopropan-2-yl)carbamoyl)phenyl)-20-carboxy- | |
| | 29-carboxylato-10-(3-guanidinopropyl)-3,9,12,15,18,26-hexaoxo- | |
| | 2,8,11,16,19,25-hexaazanonacosan-29-yl)-1,4,7,10-tetraazacyclododecane- | |
| | 1,4,7-triyl)triacetate | |
| 90 | 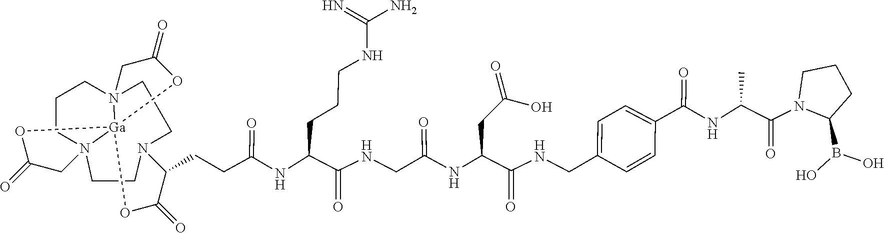 | 1054 (M − H₂O + H) |
| | Gallium(III) 2,2′-(7-((4S,10S,15R)-1-(4-(((R)-1-((R)-2-boronopyrrolidin-1- | |
| | yl)-1-oxopropan-2-yl)carbamoyl)phenyl)-15-carboxylato-4- | |
| | (carboxymethyl)-10-(3-guanidinopropyl)-3,6,9,12-tetraoxo-2,5,8,11- | |
| | tetraazapentadecan-15-yl)-1,4,7-triazonane-1,4-diyl)diacetate | |
| 91 | 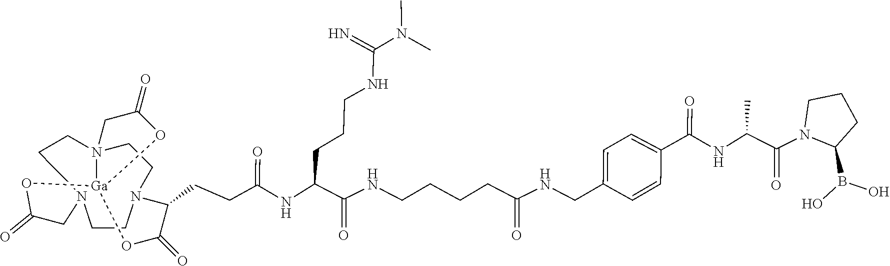 | 1027 (M + H) |
| | Gallium(III) 2,2′-(7-((R)-4-(((S)-17-(4-(((R)-1-((R)-2-boronopyrrolidin-1- | |
| | yl)-1-oxopropan-2-yl)carbamoyl)phenyl)-3-imino-2-methyl-9,15-dioxo- | |
| | 2,4,10,16-tetraazaheptadecan-8-yl)amino)-1-carboxylato-4-oxobutyl)-1,4,7- | |
| | triazonane-1,4-diyl)diacetate | |
| 92 | 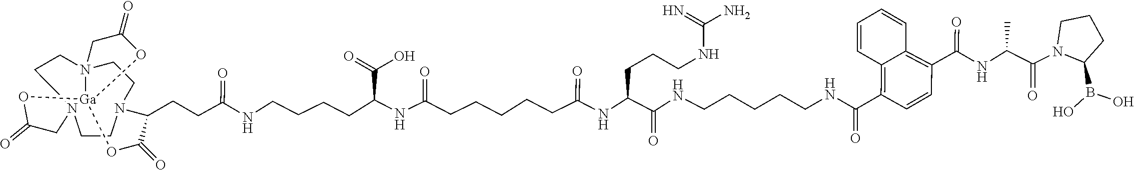 | [1302 (M − H₂O + H)] |
| | Gallium(III) 2,2′-(7-((10S,20S,29R)-1-(4-(((R)-1-((R)-2-boronopyrrolidin-1- | |
| | yl)-1-oxopropan-2-yl)carbamoyl)naphthalen-1-yl)-20-carboxy-29- | |
| | carboxylato-10-(3-guanidinopropyl)-1,9,12,18,26-pentaoxo-2,8,11,19,25- | |
| | pentaazanonacosan-29-yl)-1,4,7-triazonane-1,4-diyl)diacetate | |
| 93 | 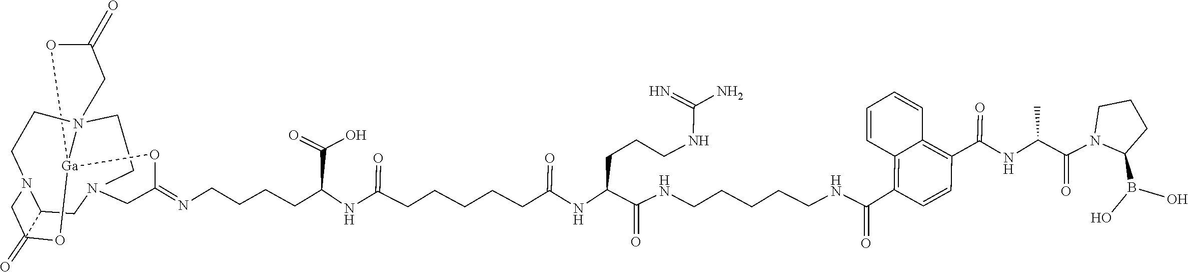 | 1247 (M + H) |
| | Gallium(III) 2,2′-(7-((10S,20S,Z)-1-(4-(((R)-1-((R)-2-boronopyrrolidin-1- | |
| | yl)-1-oxopropan-2-yl)carbamoyl)naphthalen-1-yl)-20-carboxy-10-(3- | |
| | guanidinopropyl)-26-oxido-1,9,12,18-tetraoxo-2,8,11,19,25- | |
| | pentaazaheptacos-25-en-27-yl)-1,4,7-triazonane-1,4-diyl)diacetate | |
| 94 | 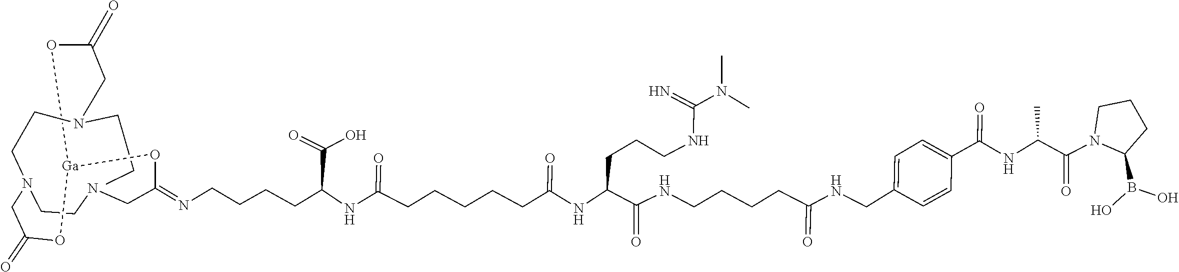 | 1225 (M + H) |
| | Gallium(III) 2,2′-(7-((10S,20S,Z)-1-(4-(((R)-1-((R)-2-boronopyrrolidin-1- | |
| | yl)-1-oxopropan-2-yl)carbamoyl)phenyl)-20-carboxy-10-(3-(3,3- | |
| | dimethylguanidino)propyl)-26-oxido-3,9,12,18-tetraoxo-2,8,11,19,25- | |
| | pentaazaheptacos-25-en-27-yl)-1,4,7-triazonane-1,4-diyl)diacetate | |
| 95 | 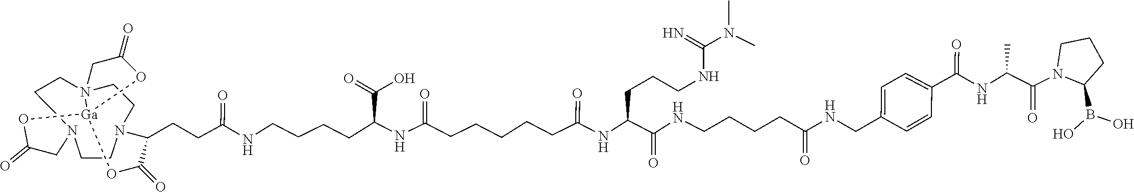 | 1279 (M − H₂O + H) |
| | Gallium(III) 2,2′-(7-((10S,20S,29R)-1-(4-(((R)-1-((R)-2-boronopyrrolidin-1- | |
| | yl)-1-oxopropan-2-yl)carbamoyl)phenyl)-20-carboxy-29-carboxylato-10-(3- | |
| | (3,3-dimethylguanidino)propyl)-3,9,12,18,26-pentaoxo-2,8,11,19,25- | |
| | pentaazanonacosan-29-yl)-1,4,7-triazonane-1,4-diyl)diacetate | |
| 96 | 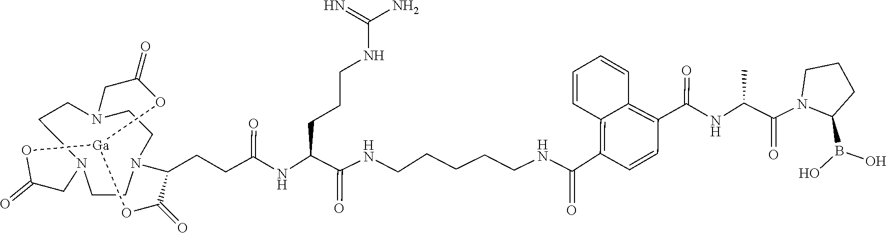 | 1050 (M + H) |
| | Gallium(III) 2,2′-(7-((R)-4-(((S)-1-((5-(4-(((R)-1-((R)-2-boronopyrrolidin-1- | |
| | yl)-1-oxopropan-2-yl)carbamoyl)-1-naphthamido)pentyl)amino)-5- | |
| | guanidino-1-oxopentan-2-yl)amino)-1-carboxylato-4-oxobutyl)-1,4,7- | |
| | triazonane-1,4-diyl)diacetate | |
| 97 | 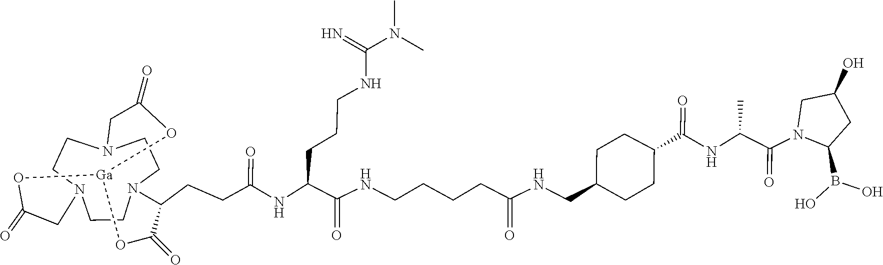 | 1049 (M + H) |
| | Gallium(III) 2,2′-(7-((R)-4-(((S)-17-((1R,4S)-4-(((R)-1-((2R,4S)-2-borono-4- | |
| | hydroxypyrrolidin-1-yl)-1-oxopropan-2-yl)carbamoyl)cyclohexyl)-3-imino- | |
| | 2-methyl-9,15-dioxo-2,4,10,16-tetraazaheptadecan-8-yl)amino)-1- | |
| | carboxylato-4-oxobutyl)-1,4,7-triazonane-1,4-diyl)diacetate | |
| 98 | 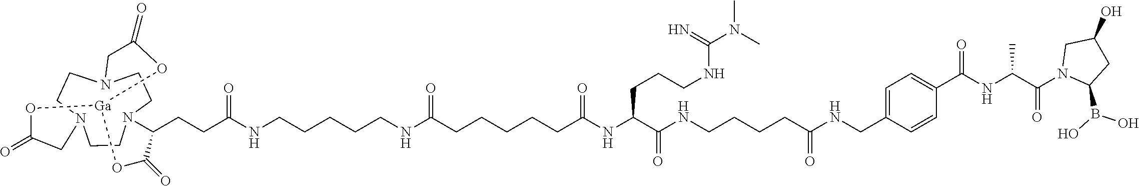 | 1269 (M + H) |
| | Gallium(III) 2,2′-(7-((10S,29R)-1-(4-(((R)-1-((2R,4S)-2-borono-4- | |
| | hydroxypyrrolidin-1-yl)-1-oxopropan-2-yl)carbamoyl)phenyl)-29- | |
| | carboxylato-10-(3-(3,3-dimethylguanidino)propyl)-3,9,12,18,26-pentaoxo- | |
| | 2,8,11,19,25-pentaazanonacosan-29-yl)-1,4,7-triazonane-1,4-diyl)diacetate | |
| 99 | 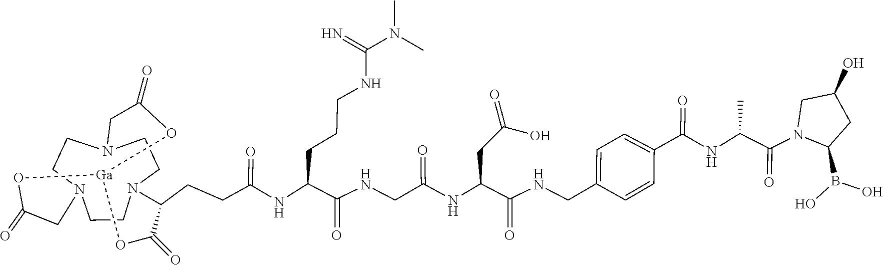 | 1116 (M + H) |
| | Gallium(III) 2,2′-(7-((4S,10S,15R)-1-(4-(((R)-1-((2R,4S)-2-borono-4- | |
| | hydroxypyrrolidin-1-yl)-1-oxopropan-2-yl)carbamoyl)phenyl)-15- | |
| | carboxylato-4-(carboxymethyl)-10-(3-(3,3-dimethylguanidino)propyl)- | |
| | 3,6,9,12-tetraoxo-2,5,8,11-tetraazapentadecan-15-yl)-1,4,7-triazonane-1,4- | |
| | diyl)diacetate | |
| 100 | 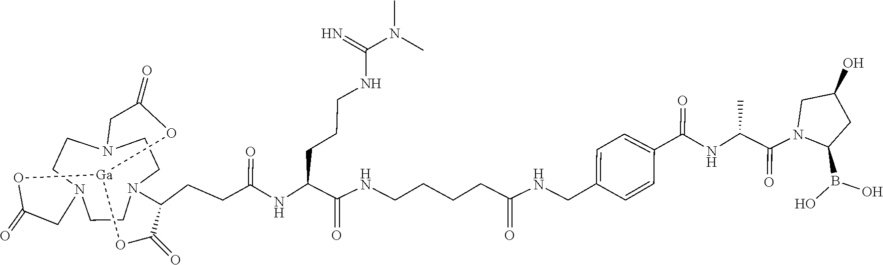 | 1042 (M + H) |
| | Gallium(III) 2,2′-(7-((R)-4-(((S)-17-(4-(((R)-1-((2R,4S)-2-borono-4- | |
| | hydroxypyrrolidin-1-yl)-1-oxopropan-2-yl)carbamoyl)phenyl)-3-imino-2- | |
| | methyl-9,15-dioxo-2,4,10,16-tetraazaheptadecan-8-yl)amino)-1-carboxylato- | |
| | 4-oxobutyl)-1,4,7-triazonane-1,4-diyl)diacetate | |

The following compounds in Table 28 were synthesized in essentially the same manner as SP-8 except using TbCl₃ instead of LuCl₃.

TABLE 28

| Ex # | Structure | ESI/MS |
|---|---|---|
| 101 | 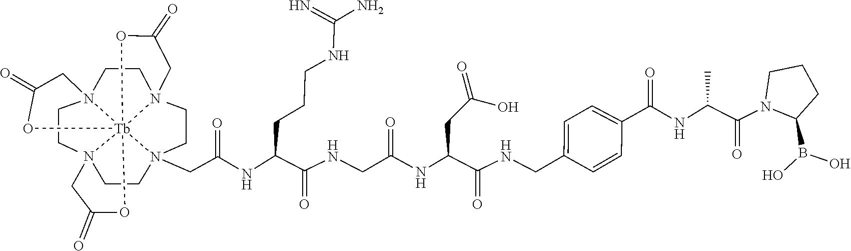 | 1173 (M − H₂O + H) |
| | Terbium(III) 2,2′,2″-(10-((4S,10S)-1-(4-(((R)-1-((R)-2-boronopyrrolidin-1- | |
| | yl)-1-oxopropan-2-yl)carbamoyl)phenyl)-4-(carboxymethyl)-10-(3- | |
| | guanidinopropyl)-3,6,9,12-tetraoxo-2,5,8,11-tetraazatridecan-13-yl)- | |
| | 1,4,7,10-tetraazacyclododecane-1,4,7-triyl)triacetate | |
| 102 | 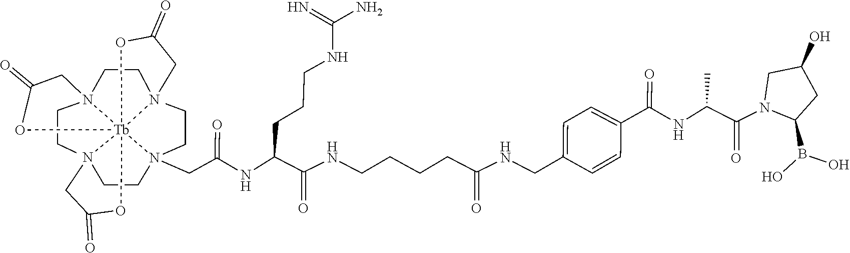 | 1134 (M + H) |
| | Terbium(III) 2,2′,2″-(10-(2-(((S)-1-((5-((4-(((R)-1-((2R,4S)-2-borono-4- | |
| | hydroxypyrrolidin-1-yl)-1-oxopropan-2-yl)carbamoyl)benzyl)amino)-5- | |
| | oxopentyl)amino)-5-guanidino-1-oxopentan-2-yl)amino)-2-oxoethyl)- | |
| | 1,4,7,10-tetraazacyclododecane-1,4,7-triyl)triacetate | |
| 103 | 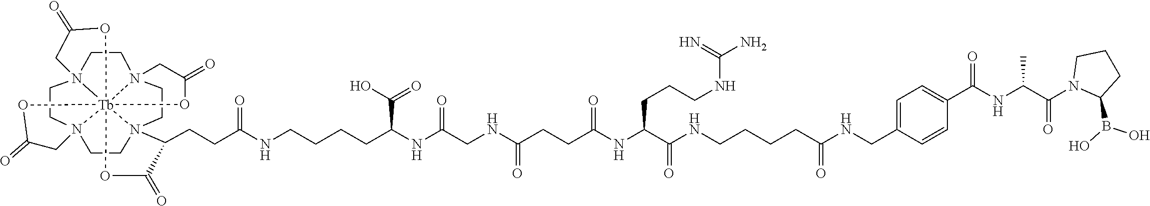 | 1457 (M − H₂O + H) |
| | Terbium(III) 2,2′,2″-(10-((10S,20S,29R)-1-(4-(((R)-1-((R)-2- | |
| | boronopyrrolidin-1-yl)-1-oxopropan-2-yl)carbamoyl)phenyl)-20-carboxy- | |
| | 29-carboxylato-10-(3-guanidinopropyl)-3,9,12,15,18,26-hexaoxo- | |
| | 2,8,11,16,19,25-hexaazanonacosan-29-yl)-1,4,7,10-tetraazacyclododecane- | |
| | 1,4,7-triyl)triacetate | |

The following compounds in Table 29 were synthesized in essentially the same manner as SP-8 except using CuCl₂ instead of LuCl₃ and the reaction was conducted at room temperature instead of 90° C.

TABLE 29

| Ex | | ESI/ |
|---|---|---|
| # | Structure | MS |
| 104 | 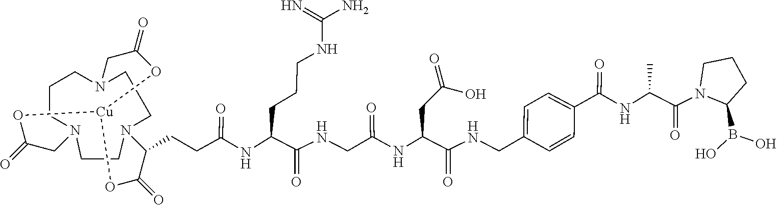 | 1049 (M − H₂O + H) |
| | Copper(II) 2,2′-(7-((4S,10S,15R)-1-(4-(((R)-1-((R)-2-boronopyrrolidin-1- | |
| | yl)-1-oxopropan-2-yl)carbamoyl)phenyl)-15-carboxylato-4- | |
| | (carboxymethyl)-10-(3-guanidinopropyl)-3,6,9,12-tetraoxo-2,5,8,11- | |
| | tetraazapentadecan-15-yl)-1,4,7-triazonane-1,4-diyl)diacetate | |
| 105 | 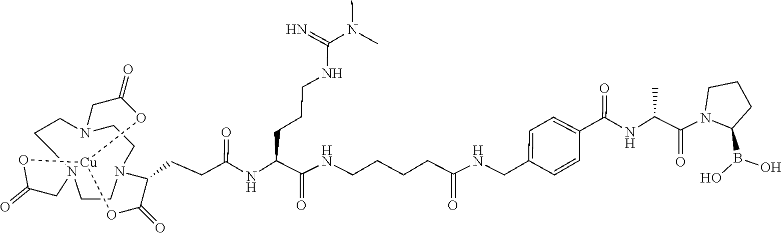 | 1004 (M − H₂O + H) |
| | Copper(II) 2,2′-(7-((R)-4-(((S)-17-(4-(((R)-1-((R)-2-boronopyrrolidin-1-yl)- | |
| | 1-oxopropan-2-yl)carbamoyl)phenyl)-3-imino-2-methyl-9,15-dioxo- | |
| | 2,4,10,16-tetraazaheptadecan-8-yl)amino)-1-carboxylato-4-oxobutyl)-1,4,7- | |
| | triazonane-1,4-diyl)diacetate | |
| 106 | 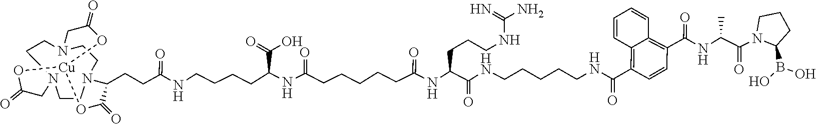 | 1296 (M − H₂O + H) |
| | Copper(II) 2,2′-(7-((10S,20S,29R)-1-(4-(((R)-1-((R)-2-boronopyrrolidin-1- | |
| | yl)-1-oxopropan-2-yl)carbamoyl)naphthalen-1-yl)-20-carboxy-29- | |
| | carboxylato-10-(3-guanidinopropyl)-1,9,12,18,26-pentaoxo-2,8,11,19,25- | |
| | pentaazanonacosan-29-yl)-1,4,7-triazonane-1,4-diyl)diacetate | |
| 107 | 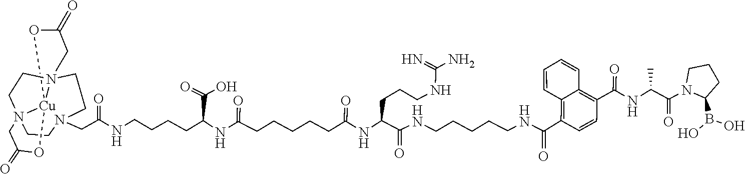 | 1224 (M − H₂O + H) |
| | Copper(II) 2,2′-(7-((10S,20S,Z)-1-(4-(((R)-1-((R)-2-boronopyrrolidin-1-yl)- | |
| | 1-oxopropan-2-yl)carbamoyl)naphthalen-1-yl)-20-carboxy-10-(3- | |
| | guanidinopropyl)-26-oxido-1,9,12,18-tetraoxo-2,8,11,19,25- | |
| | pentaazaheptacos-25-en-27-yl)-1,4,7-triazonane-1,4-diyl)diacetate | |
| 108 | 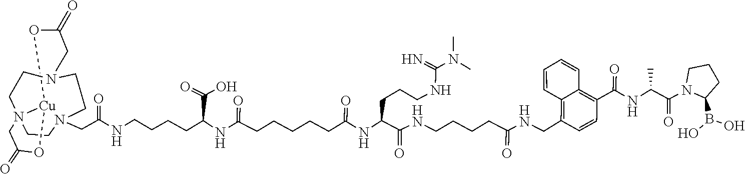 | 1202 (M − H₂O + H) |
| | Copper(II) 2,2′-(7-((10S,20S,Z)-1-(4-(((R)-1-((R)-2-boronopyrrolidin-1-yl)- | |
| | 1-oxopropan-2-yl)carbamoyl)phenyl)-20-carboxy-10-(3-(3,3- | |
| | dimethylguanidino)propyl)-26-oxido-3,9,12,18-tetraoxo-2,8,11,19,25- | |
| | pentaazaheptacos-25-en-27-yl)-1,4,7-triazonane-1,4-diyl)diacetate | |
| 109 | 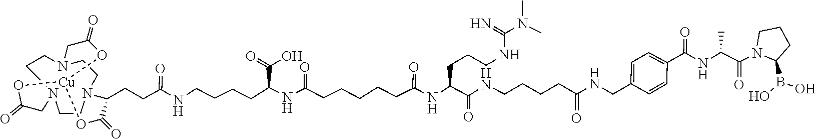 | 1274 (M − H₂O + H) |
| | Copper(II) 2,2′-(7-((10S,20S,29R)-1-(4-(((R)-1-((R)-2-boronopyrrolidin-1- | |
| | yl)-1-oxopropan-2-yl)carbamoyl)phenyl)-20-carboxy-29-carboxylato-10-(3- | |
| | (3,3-dimethylguanidino)propyl)-3,9,12,18,26-pentaoxo-2,8,11,19,25- | |
| | pentaazanonacosan-29-yl)-1,4,7-triazonane-1,4-diyl)diacetate | |
| 110 | 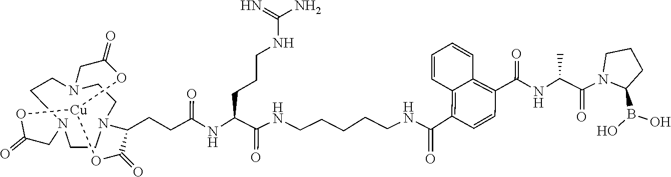 | 1026 (M − H₂O + H) |
| | Copper(II) 2,2′-(7-((R)-4-(((S)-1-((5-(4-(((R)-1-((R)-2-boronopyrrolidin-1- | |
| | yl)-1-oxopropan-2-yl)carbamoyl)-1-naphthamido)pentyl)amino)-5- | |
| | guanidino-1-oxopentan-2-yl)amino)-1-carboxylato-4-oxobutyl)-1,4,7- | |
| | triazonane-1,4-diyl)diacetate | |
| 111 | 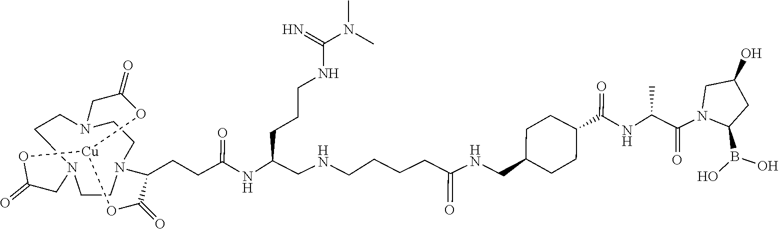 | 1025 (M − H₂O + H) |
| | Copper(II) 2,2′-(7-((R)-4-(((S)-17-((1R,4S)-4-(((R)-1-((2R,4S)-2-borono-4- | |
| | hydroxypyrrolidin-1-yl)-1-oxopropan-2-yl)carbamoyl)cyclohexyl)-3-imino- | |
| | 2-methyl-9,15-dioxo-2,4,10,16-tetraazaheptadecan-8-yl)amino)-1- | |
| | carboxylato-4-oxobutyl)-1,4,7-triazonane-1,4-diyl)diacetate | |
| 112 | 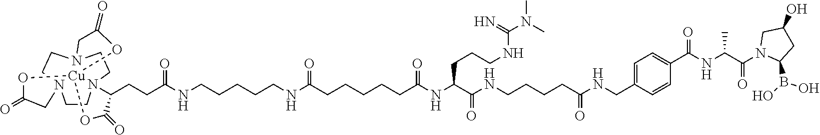 | 1246 (M − H₂O + H) |
| | Copper(II) 2,2′-(7-((10S,29R)-1-(4-(((R)-1-((2R,4S)-2-borono-4- | |
| | hydroxypyrrolidin-1-yl)-1-oxopropan-2-yl)carbamoyl)phenyl)-29- | |
| | carboxylato-10-(3-(3,3-dimethylguanidino)propyl)-3,9,12,18,26-pentaoxo- | |
| | 2,8,11,19,25-pentaazanonacosan-29-yl)-1,4,7-triazonane-1,4-diyl)diacetate | |
| 113 | 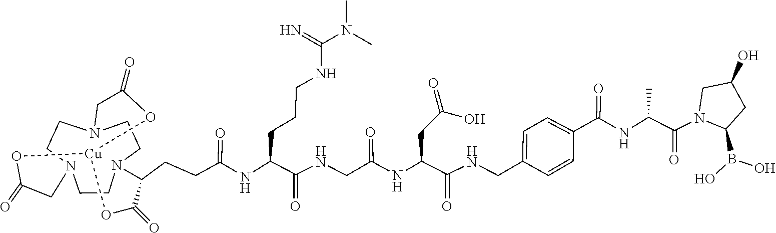 | 1092 (M − H₂O + H) |
| | Copper(II) 2,2′-(7-((4S,10S,15R)-1-(4-(((R)-1-((2R,4S)-2-borono-4- | |
| | hydroxypyrrolidin-1-yl)-1-oxopropan-2-yl)carbamoyl)phenyl)-15- | |
| | carboxylato-4-(carboxymethyl)-10-(3-(3,3-dimethylguanidino)propyl)- | |
| | 3,6,9,12-tetraoxo-2,5,8,11-tetraazapentadecan-15-yl)-1,4,7-triazonane-1,4- | |
| | diyl)diacetate | |
| 114 | 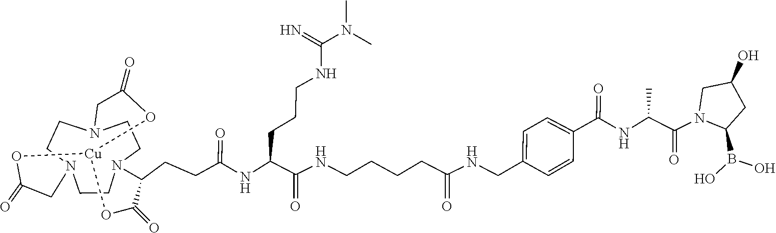 | 1019 (M − H₂O + H) |
| | Copper(II) 2,2′-(7-((R)-4-(((S)-17-(4-(((R)-1-((2R,4S)-2-borono-4- | |
| | hydroxypyrrolidin-1-yl)-1-oxopropan-2-yl)carbamoyl)phenyl)-3-imino-2- | |
| | methyl-9,15-dioxo-2,4,10,16-tetraazaheptadecan-8-yl)amino)-1-carboxylato- | |
| | 4-oxobutyl)-1,4,7-triazonane-1,4-diyl)diacetate | |

The following compounds in Table 30 were synthesized in essentially the same manner as SP-8 except using BiCl₃ instead of LuCl₃.

TABLE 30

| Ex | | ESI/ |
|---|---|---|
| # | Structure | MS |
| 115 | 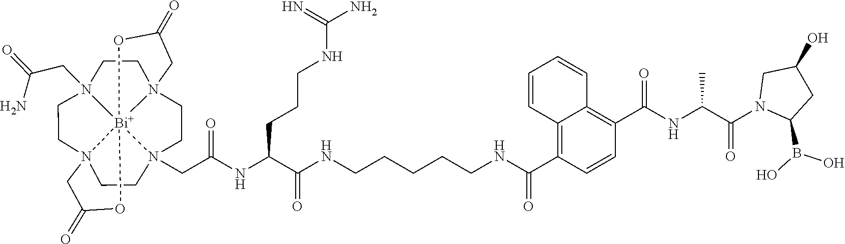 | 1233 (M + H) |
| | Bismuth(III) 2,2′-(4-(2-amino-2-oxoethyl)-10-(2-(((S)-1-((5-(4-(((R)-1- | |
| | ((2R,4S)-2-borono-4-hydroxypyrrolidin-1-yl)-1-oxopropan-2-yl)carbamoyl)- | |
| | 1-naphthamido)pentyl)amino)-5-guanidino-1-oxopentan-2-yl)amino)-2- | |
| | oxoethyl)-1,4,7,10-tetraazacyclododecane-1,7-diyl)diacetate | |
| 116 | 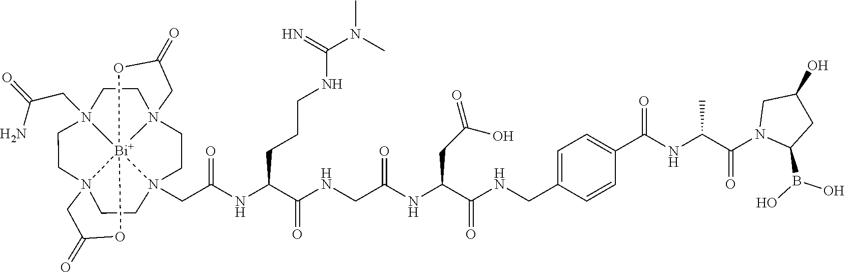 | 1284 (M + H) |
| | Bismuth(III) 2,2′-(4-(2-amino-2-oxoethyl)-10-((4S,10S)-1-(4-(((R)-1- | |
| | ((2R,4S)-2-borono-4-hydroxypyrrolidin-1-yl)-1-oxopropan-2- | |
| | yl)carbamoyl)phenyl)-4-(carboxymethyl)-10-(3-(3,3- | |
| | dimethylguanidino)propyl)-3,6,9,12-tetraoxo-2,5,8,11-tetraazatridecan-13- | |
| | yl)-1,4,7,10-tetraazacyclododecane-1,7-diyl)diacetate | |
| 117 | 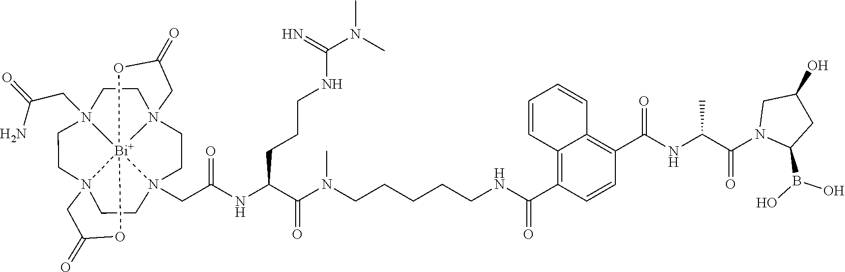 | 1275 (M + H) |
| | Bismuth(III) 2,2′-(4-(2-amino-2-oxoethyl)-10-(2-(((S)-17-(4-(((R)-1- | |
| | ((2R,4S)-2-borono-4-hydroxypyrrolidin-1-yl)-1-oxopropan-2- | |
| | yl)carbamoyl)naphthalen-1-yl)-3-imino-2,10-dimethyl-9,17-dioxo-2,4,10,16- | |
| | tetraazaheptadecan-8-yl)amino)-2-oxoethyl)-1,4,7,10- | |
| | tetraazacyclododecane-1,7-diyl)diacetate | |
| 118 | 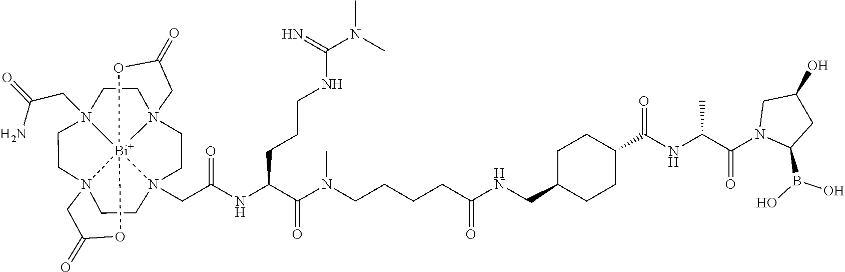 | 1231 (M + H) |
| | Bismuth(III) 2,2′-(4-(2-amino-2-oxoethyl)-10-(2-(((S)-17-((1R,4S)-4-(((R)- | |
| | 1-((2R,4S)-2-borono-4-hydroxypyrrolidin-1-yl)-1-oxopropan-2- | |
| | yl)carbamoyl)cyclohexyl)-3-imino-2,10-dimethyl-9,15-dioxo-2,4,10,16- | |
| | tetraazaheptadecan-8-yl)amino)-2-oxoethyl)-1,4,7,10- | |
| | tetraazacyclododecane-1,7-diyl)diacetate | |
| 119 | 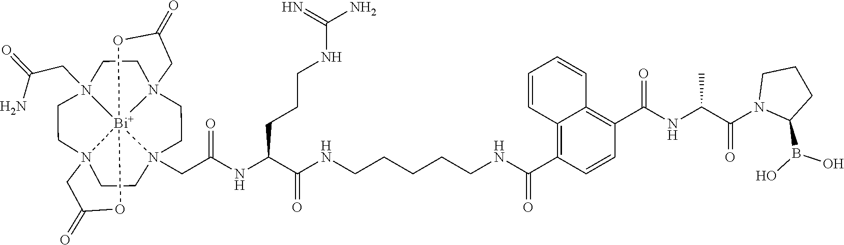 | 1217 (M + H) |
| | Bismuth(III) 2,2′-(4-(2-amino-2-oxoethyl)-10-(2-(((S)-1-((5-(4-(((R)-1-((R)- | |
| | 2-boronopyrrolidin-1-yl)-1-oxopropan-2-yl)carbamoyl)-1- | |
| | naphthamido)pentyl)amino)-5-guanidino-1-oxopentan-2-yl)amino)-2- | |
| | oxoethyl)-1,4,7,10-tetraazacyclododecane-1,7-diyl)diacetate | |

### Example 120

### Lead(II) 2,2′-(4-(2-amino-2-oxoethyl)-10-(2-(((S)-1-((5-((4-(((R)-1-((R)-2-boronopyrrolidin-1-yl)-1-oxopropan-2-yl)carbamoyl)benzyl)amino)-5-oxopentyl)amino)-5-guanidino-1-oxopentan-2-yl)amino)-2-oxoethyl)-1,4,7,10-tetraazacyclododecane-1,7-diyl)diacetate

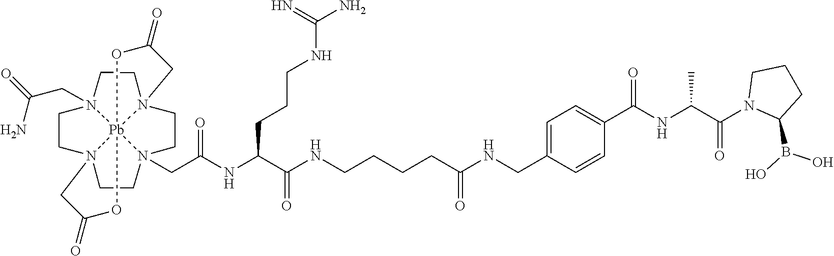

2,2′-(4-(2-Amino-2-oxoethyl)-10-(2-(((S)-1-((5-((4-(((R)-1-((R)-2-boronopyrrolidin-1-yl)-1-oxopropan-2-yl)carbamoyl)benzyl)amino)-5-oxopentyl)amino)-5-guanidino-1-oxopentan-2-yl)amino)-2-oxoethyl)-1,4,7,10-tetraazacyclododecane-1,7-diyl)diacetic acid was reacted with Pb(NO₃)₂ according to SP-7 to afford the title compound. ESI/MS m/z: 1166 (M+H)

The following compounds in Table X were synthesized in essentially the same manner as described for lead(II) 2,2′-(4-(2-amino-2-oxoethyl)-10-(2-(((S)-1-((5-((4-(((R)-1-((R)-2-boronopyrrolidin-1-yl)-1-oxopropan-2-yl)carbamoyl)benzyl)amino)-5-oxopentyl)amino)-5-guanidino-1-oxopentan-2-yl)amino)-2-oxoethyl)-1,4,7,10-tetraazacyclododecane-1,7-diyl)diacetate.

TABLE 31

| Ex | | ESI/ |
|---|---|---|
| # | Structure | MS |
| 121 | 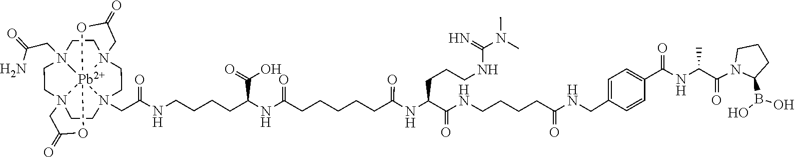 | 1463 (M + H) |
| | Lead(II) N2-(7-(((S)-17-(4-(((R)-1-((R)-2-boronopyrrolidin-1-yl)-1- | |
| | oxopropan-2-yl)carbamoyl)phenyl)-3-imino-2-methyl-9,15-dioxo-2,4,10,16- | |
| | tetraazaheptadecan-8-yl)amino)-7-oxoheptanoyl)-N6-(2-(4,7,10-tris(2- | |
| | amino-2-oxoethyl)-1,4,7,10-tetraazacyclododecan-1-yl)acetyl)-L-lysine | |
| 122 | 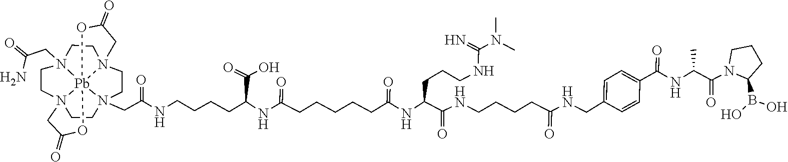 | 1463 (M + H) |
| | Lead(II) 2,2′-(4-(2-amino-2-oxoethyl)-10-((10S,20S)-1-(4-(((R)-1-((R)-2- | |
| | boronopyrrolidin-1-yl)-1-oxopropan-2-yl)carbamoyl)phenyl)-20-carboxy- | |
| | 10-(3-(3,3-dimethylguanidino)propyl)-3,9,12,18,26-pentaoxo-2,8,11,19,25- | |
| | pentaazaheptacosan-27-yl)-1,4,7,10-tetraazacyclododecane-1,7- | |
| | diyl)diacetate | |
| 123 | 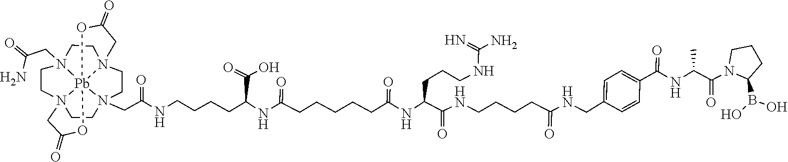 | |
| | Lead(II) 2,2′-(4-(2-amino-2-oxoethyl)-10-((10S,20S)-1-(4-(((R)-1-((R)-2- | |
| | boronopyrrolidin-1-yl)-1-oxopropan-2-yl)carbamoyl)phenyl)-20-carboxy- | |
| | 10-(3-guanidinopropyl)-3,9,12,18,26-pentaoxo-2,8,11,19,25- | |
| | pentaazaheptacosan-27-yl)-1,4,7,10-tetraazacyclododecane-1,7- | |
| | diyl)diacetate | |
| 124 | 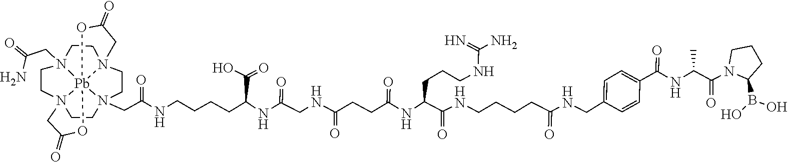 | 1433 (M − H₂O + H) |
| | Lead(II) 2,2′-(4-(2-amino-2-oxoethyl)-10-((10S,20S)-1-(4-(((R)-1-((R)-2- | |
| | boronopyrrolidin-1-yl)-1-oxopropan-2-yl)carbamoyl)phenyl)-20-carboxy- | |
| | 10-(3-guanidinopropyl)-3,9,12,15,18,26-hexaoxo-2,8,11,16,19,25- | |
| | hexaazaheptacosan-27-yl)-1,4,7,10-tetraazacyclododecane-1,7-diyl)diacetate | |
| 125 | 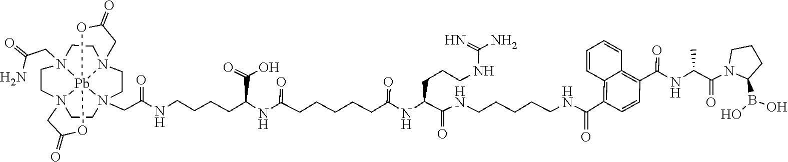 | 1468 (M − H₂O + H) |
| | Lead(II) 2,2′-(4-(2-amino-2-oxoethyl)-10-((10S,20S)-1-(4-(((R)-1-((R)-2- | |
| | boronopyrrolidin-1-yl)-1-oxopropan-2-yl)carbamoyl)naphthalen-1-yl)-20- | |
| | carboxy-10-(3-guanidinopropyl)-1,9,12,18,26-pentaoxo-2,8,11,19,25- | |
| | pentaazaheptacosan-27-yl)-1,4,7,10-tetraazacyclododecane-1,7- | |
| | diyl)diacetate | |
| 126 | 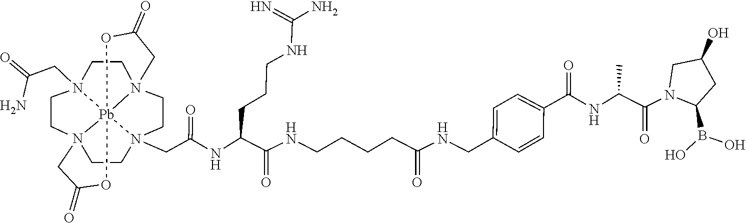 | 1182 (M + H) |
| | Lead(II) 2,2′-(4-(2-amino-2-oxoethyl)-10-(2-(((S)-1-((5-((4-(((R)-1- | |
| | ((2R,4S)-2-borono-4-hydroxypyrrolidin-1-yl)-1-oxopropan-2- | |
| | yl)carbamoyl)benzyl)amino)-5-oxopentyl)amino)-5-guanidino-1-oxopentan- | |
| | 2-yl)amino)-2-oxoethyl)-1,4,7,10-tetraazacyclododecane-1,7-diyl)diacetate | |
| 127 | 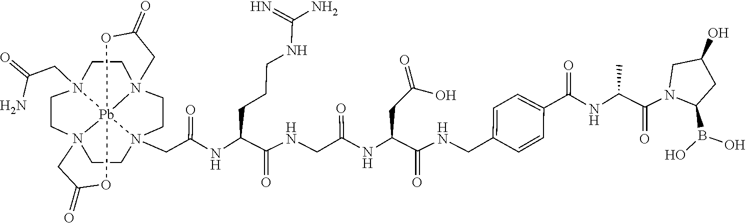 | 1255 (M + H) |
| | Lead(II) 2,2′-(4-(2-amino-2-oxoethyl)-10-((4S,10S)-1-(4-(((R)-1-((2R,4S)-2- | |
| | borono-4-hydroxypyrrolidin-1-yl)-1-oxopropan-2-yl)carbamoyl)phenyl)-4- | |
| | (carboxymethyl)-10-(3-guanidinopropyl)-3,6,9,12-tetraoxo-2,5,8,11- | |
| | tetraazatridecan-13-yl)-1,4,7,10-tetraazacyclododecane-1,7-diyl)diacetate | |
| 128 | 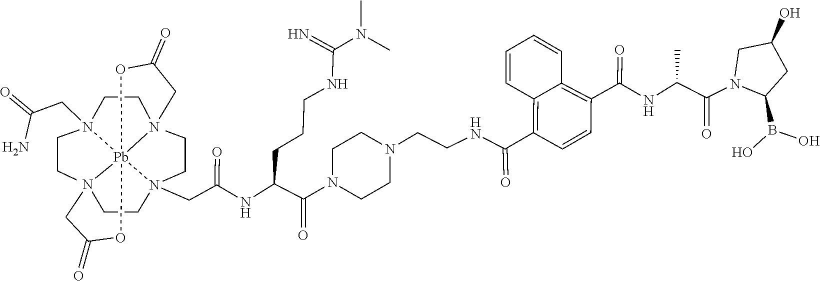 | 1287 (M + H) |
| | Lead(II) 2,2′-(4-(2-amino-2-oxoethyl)-10-(2-(((S)-1-(4-(2-(4-(((R)-1- | |
| | ((2R,4S)-2-borono-4-hydroxypyrrolidin-1-yl)-1-oxopropan-2-yl)carbamoyl)- | |
| | 1-naphthamido)ethyl)piperazin-1-yl)-5-(3,3-dimethylguanidino)-1- | |
| | oxopentan-2-yl)amino)-2-oxoethyl)-1,4,7,10-tetraazacyclododecane-1,7- | |
| | diyl)diacetate | |
| 129 | 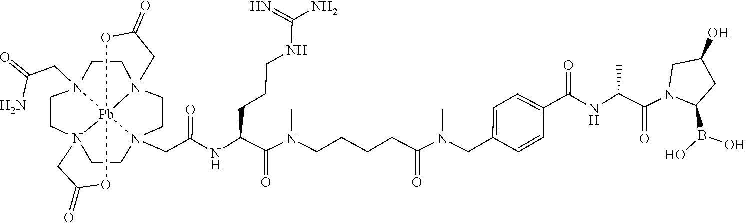 | 1210 (M + H) |
| | Lead(II) 2,2′-(4-(2-amino-2-oxoethyl)-10-(2-(((S)-1-((5-((4-(((R)-1- | |
| | ((2R,4S)-2-borono-4-hydroxypyrrolidin-1-yl)-1-oxopropan-2- | |
| | yl)carbamoyl)benzyl)(methyl)amino)-5-oxopentyl)(methyl)amino)-5- | |
| | guanidino-1-oxopentan-2-yl)amino)-2-oxoethyl)-1,4,7,10- | |
| | tetraazacyclododecane-1,7-diyl)diacetate | |
| 130 | 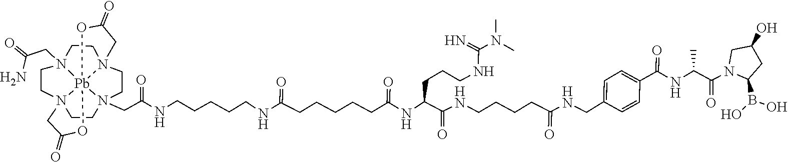 | 710 [(M − H₂O)/ 2 + H) |
| | Lead(II) 2,2′-(4-(2-amino-2-oxoethyl)-10-((S)-1-(4-(((R)-1-((2R,4S)-2- | |
| | borono-4-hydroxypyrrolidin-1-yl)-1-oxopropan-2-yl)carbamoyl)phenyl)-10- | |
| | (3-(3,3-dimethylguanidino)propyl)-3,9,12,18,26-pentaoxo-2,8,11,19,25- | |
| | pentaazaheptacosan-27-yl)-1,4,7,10-tetraazacyclododecane-1,7- | |
| | diyl)diacetate | |
| 131 | 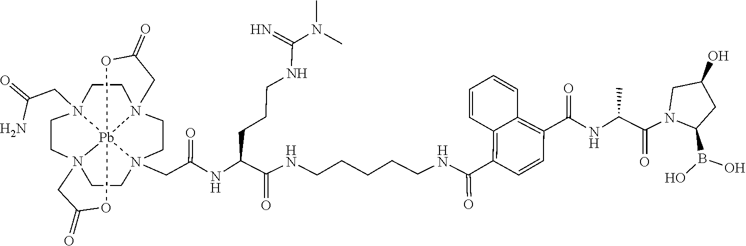 | 1260 (M + H) |
| | Lead(II) 2,2′-(4-(2-amino-2-oxoethyl)-10-(2-(((S)-17-(4-(((R)-1-((2R,4S)-2- | |
| | borono-4-hydroxypyrrolidin-1-yl)-1-oxopropan-2-yl)carbamoyl)naphthalen- | |
| | 1-yl)-3-imino-2-methyl-9,17-dioxo-2,4,10,16-tetraazaheptadecan-8- | |
| | yl)amino)-2-oxoethyl)-1,4,7,10-tetraazacyclododecane-1,7-diyl)diacetate | |
| 132 | 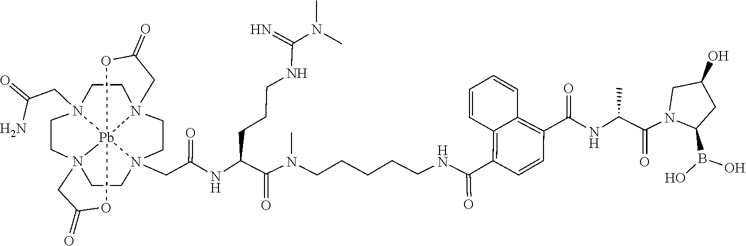 | 1274 (M + H) |
| | Lead(II) 2,2′-(4-(2-amino-2-oxoethyl)-10-(2-(((S)-17-(4-(((R)-1-((2R,4S)-2- | |
| | borono-4-hydroxypyrrolidin-1-yl)-1-oxopropan-2-yl)carbamoyl)naphthalen- | |
| | 1-yl)-3-imino-2,10-dimethyl-9,17-dioxo-2,4,10,16-tetraazaheptadecan-8- | |
| | yl)amino)-2-oxoethyl)-1,4,7,10-tetraazacyclododecane-1,7-diyl)diacetate | |
| 133 | 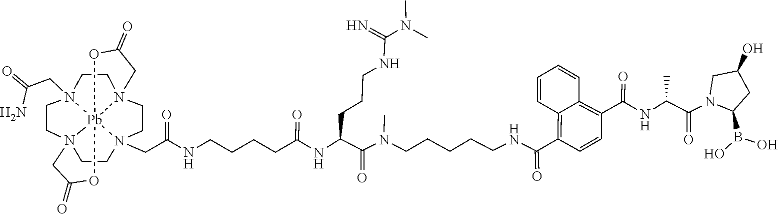 | 678 ((M − H₂O)/ 2 + H) |
| | Lead(II) 2,2′-(4-(2-amino-2-oxoethyl)-10-((S)-1-(4-(((R)-1-((2R,4S)-2- | |
| | borono-4-hydroxypyrrolidin-1-yl)-1-oxopropan-2-yl)carbamoyl)naphthalen- | |
| | 1-yl)-10-(3-(3,3-dimethylguanidino)propyl)-8-methyl-1,9,12,18-tetraoxo- | |
| | 2,8,11,17-tetraazanonadecan-19-yl)-1,4,7,10-tetraazacyclododecane-1,7- | |
| | diyl)diacetate | |
| 134 | 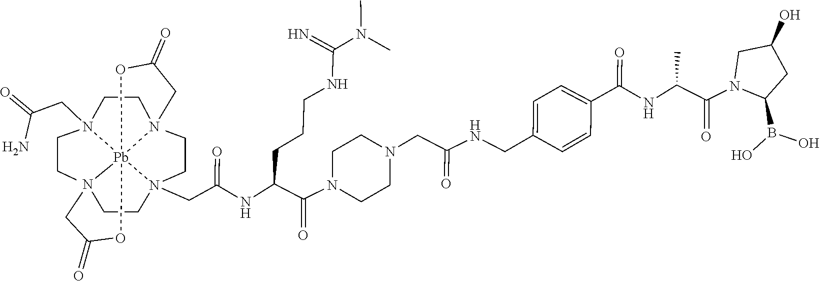 | 1237 (M + H) |
| | Lead(II) 2,2′-(4-(2-amino-2-oxoethyl)-10-(2-(((S)-1-(4-(2-((4-(((R)-1- | |
| | ((2R,4S)-2-borono-4-hydroxypyrrolidin-1-yl)-1-oxopropan-2- | |
| | yl)carbamoyl)benzyl)amino)-2-oxoethyl)piperazin-1-yl)-5-(3,3- | |
| | dimethylguanidino)-1-oxopentan-2-yl)amino)-2-oxoethyl)-1,4,7,10- | |
| | tetraazacyclododecane-1,7-diyl)diacetate | |
| 135 | 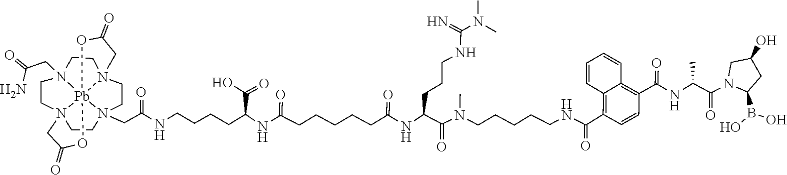 | 1526 (M − H₂O + H) |
| | Lead(II) 2,2′-(4-(2-amino-2-oxoethyl)-10-((10S,20S)-1-(4-(((R)-1-((2R,4S)- | |
| | 2-borono-4-hydroxypyrrolidin-1-yl)-1-oxopropan-2- | |
| | yl)carbamoyl)naphthalen-1-yl)-20-carboxy-10-(3-(3,3- | |
| | dimethylguanidino)propyl)-8-methyl-1,9,12,18,26-pentaoxo-2,8,11,19,25- | |
| | pentaazaheptacosan-27-yl)-1,4,7,10-tetraazacyclododecane-1,7- | |
| | diyl)diacetate | |
| 136 | 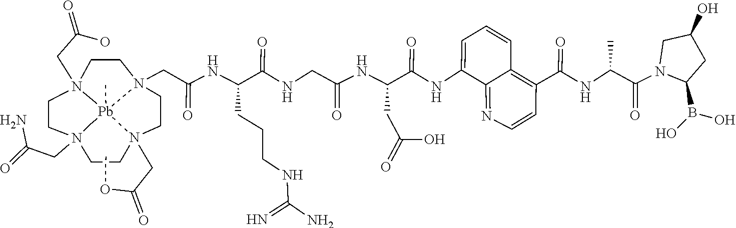 | 1274 (M − H₂O + H) |
| | Lead(II) 2,2′-(4-(2-amino-2-oxoethyl)-10-(2-(((S)-1-((2-(((S)-1-((4-(((R)-1- | |
| | ((2R,4S)-2-borono-4-hydroxypyrrolidin-1-yl)-1-oxopropan-2- | |
| | yl)carbamoyl)quinolin-8-yl)amino)-3-carboxy-1-oxopropan-2-yl)amino)-2- | |
| | oxoethyl)amino)-5-guanidino-1-oxopentan-2-yl)amino)-2-oxoethyl)- | |
| | 1,4,7,10-tetraazacyclododecane-1,7-diyl)diacetate | |

Biological Assay: The following assay demonstrates that the exemplified compounds are FAP inhibitors.

KB-hFAP Cellular Retention Assay: A 10,000 cells/100 mL/well KB-hFAP cell suspension was prepared in assay buffer (Serum-free RPMI 1640) and plated in rows B-G of 96-well black, clear-bottom tissue culture treated plates (Falcon, Cat. No. 353219). Rows A and H received 100 mL/well of assay buffer (Serum-free RPMI 1640) alone. The assay plates were allowed to equilibrate at 37° C., 5% CO₂ for 12-16 hrs. 10 mM DMSO inhibitor stock solutions were then diluted to 1 mM in the assay buffer. The assay buffer was then removed from each assay plate and replaced with 100 mL of new assay buffer (rows A, B and H) or 1 mM of inhibitor (rows C-G). The plates were incubated at 37° C., 5% CO₂ for 1 hr. Wells in columns 1-6 were left unchanged to serve as inhibition controls over time (No Exchange). The contents of wells in columns 7-12 were aspirated and washed with 200 mL PBS (Gibco, Cat. No. 14190-136). The PBS wash was aspirated, and the wells were treated with 100 mL of assay buffer without inhibitor (+ Exchange). Plates were allowed to incubate at 37° C., 5% CO₂ for 0.5-48 hrs. A 22 mM (11×) 3144-AMC substrate (10 mM DMSO stock, Tufts University Batch ID 3144-03) working solution was prepared via dilution of the 10 mM DMSO stock into the assay buffer. At the assigned timepoints, all wells were treated with 10 mL of the working solution for a final assay concentration of 2 mM. The plates were briefly shaken and the fluorescence (Ex₃₈₀:Em₄₆₀) measured at 1 min intervals for 20 min at 37° C.

The data was analyzed with GraphPad Prism and Excel as follows:

% FAP inhibition: In GraphPad Prism, the slope (DFU/min) was determined from 5-20 min for each well. The wells in row B served as the no inhibitor controls, for which the average slope was calculated. In Excel, the no inhibitor control wells were matched to the appropriate treatment wells in either columns 1-6 (No Exchange) or 7-12 (+ Exchange). Percent FAP inhibition was calculated as follows:

$$\% FAP Inhibition = [ ( No Inhibitor Average - FAPI Treated Well ) / No Inhibitor Average ] * 100$$

The % FAP inhibition for each inhibitor without exchange at the first timepoint tested served as the 0-hour point for comparison to subsequent values following the media exchange.

In the above assay, compounds of Examples 6-35 and 37-39 were tested and exhibited FAP inhibition with a relative IC₅₀ of <30 nM. This data shows that compounds of Formula I as described herein are FAP inhibitors.

rhFAP Inhibition Assay Protocol
1) DMSO stocks (1 or 10 mM) were diluted 1:100 into assay buffer (Dulbecco's PBS without calcium or magnesium; Gibco, Cat. No. 14190136) to yield 10 or 100 mM working stocks for the dilution plate.
2) The dilution plate was prepared by adding the 10 or 100 mM stocks prepared in the previous step to row A of a 96-well plate. From this, 1:10 serial dilutions into assay buffer were performed for rows B—H.
3) Recombinant human FAP (rhFAP from R&D Systems, Cat. No. 3715-SE) was diluted into assay buffer for a working enzyme concentration of 0.22 nM (1.1×). All wells in columns 2-10 of each black, clear-bottom assay plate (Falcon, Cat. No. 353219) received 180 mL of FAP. Wells in column 1 served as the controls for the assay.
   a. Background controls: wells A1-C1 (200 mL assay buffer alone)
   b. No inhibitor controls: wells D1-F1 (180 mL FAP+20 mL assay buffer alone)
4) Enzyme pretreatment with inhibitors was carried out via addition of 20 mL of the inhibitor of interest from the dilution plate prepared in step 2 to columns 2-4, 5-7 or 8-10 of the assay plate. This allowed for testing of all inhibitors in triplicate. Inhibitors were incubated with the enzyme for 10 min at 37° C.
5) A 500 mM (20×) 3144-AMC substrate (10 mM DMSO stock, Tufts University Batch ID3144-03) working solution was prepared via dilution of the DMSO stock into assay buffer. All wells were treated with 10 mL of the working solution for a final assay concentration of 25 mM. The plates were shaken briefly and incubated for 30 min at 37° C.
6) Endpoint fluorescence was measured at Ex₃₈₀:Em₄₆₀. Data was analyzed using GraphPad Prism to calculate IC50 values which are shown in Table X.

### Data Table

TABLE 32

| Example | Recombinant Enzyme hFAP IC50 |
|---|---|
| Number | (nM)/Z-GP-AMC Substrate |
| 1 | 4.5 |
| 2 | 1.6 |
| 3 | 2.1 |
| 4 | 1.9 |
| 5 | 2.2 |
| 6 | 2.7 |
| 7 | 3.3 |
| 8 | 2.2 |
| 9 | 4.4 |
| 10 | 2.2 |
| 11 | 7.4 |
| 12 | 2.9 |
| 13 | 2.5 |
| 14 | 2.5 |
| 15 | 2.8 |
| 16 | 2.7 |
| 17 | 4.0 |
| 18 | 4.1 |
| 19 | 5.6 |
| 20 | 5.8 |
| 21 | 3.3 |
| 22 | 2.6 |
| 23 | 2.1 |
| 24 | 2.3 |
| 25 | 2.9 |
| 26 | 2.8 |
| 27 | 2.4 |
| 28 | 12.0 |
| 29 | 25.0 |
| 30 | 17.0 |
| 31 | 13.0 |
| 32 | 9.7 |
| 33 | 2.5 |
| 34 | 6.8 |
| 35 | |
| 36 | |
| 37 | 3.0 |
| 38 | 2.4 |
| 39 | 2.9 |
| 41 | 2.6 |
| 42 | 2.5 |
| 43 | 1.6 |
| 44 | 1.6 |
| 45 | 2.9 |
| 46 | 2.3 |
| 47 | 2.1 |
| 48 | 1.7 |
| 49 | 14.0 |
| 51 | 1.5 |
| 52 | 3.8 |
| 53 | 310.0 |
| 55 | 2.2 |
| 56 | 2.7 |
| 57 | 7.9 |
| 58 | 7.5 |
| 59 | |
| 60 | 12.0 |
| 61 | 7.1 |
| 62 | 2.7 |
| 63 | 3.0 |
| 64 | 2.3 |
| 65 | 3.1 |
| 66 | |
| 67 | 1.7 |
| 68 | 2.2 |
| 69 | 2.8 |
| 70 | 13.9 |
| 71 | 2.4 |
| 72 | 3.0 |
| 73 | 290.0 |
| 74 | 2.5 |
| 75 | 6.1 |
| 76 | 3600.0 |
| 77 | 6.7 |
| 78 | 5.4 |
| 79 | 3.5 |
| 80 | 2.8 |
| 81 | 3.3 |
| 82 | 6.7 |
| 83 | 8.6 |
| 84 | 2.4 |
| 85 | 5.8 |
| 86 | 2.3 |
| 87 | 6.9 |
| 88 | 320.0 |
| 89 | 2.3 |
| 90 | 2.6 |
| 91 | 15.0 |
| 92 | 4.5 |
| 93 | 5.9 |
| 94 | 2.7 |
| 95 | 2.7 |
| 96 | 8.0 |
| 97 | 6.2 |
| 98 | 2.8 |
| 99 | 1.9 |
| 100 | 6.1 |
| 101 | 2.7 |
| 102 | 2.9 |
| 103 | 3.0 |
| 104 | |
| 105 | 5.6 |
| 106 | 8.0 |
| 107 | 5.6 |
| 108 | 2.9 |
| 109 | 2.8 |
| 110 | 4.5 |
| 111 | 5.2 |
| 112 | 2.4 |
| 113 | 1.7 |
| 114 | 1.9 |
| 115 | |
| 116 | |
| 117 | |
| 118 | |
| 119 | |
| 120 | 7.1 |
| 121 | 3.2 |
| 122 | 2.6 |
| 123 | 1.1 |
| 124 | 3.3 |
| 125 | 3.7 |
| 126 | 1.6 |
| 127 | 1.9 |
| 128 | 2.4 |
| 129 | 2.6 |
| 130 | 2.9 |
| 131 | 1.8 |
| 132 | 2.0 |
| 133 | 6.1 |
| 134 | 1.8 |
| 135 | 2.9 |
| 136 | 1.6 |

1. A compound of the formula: 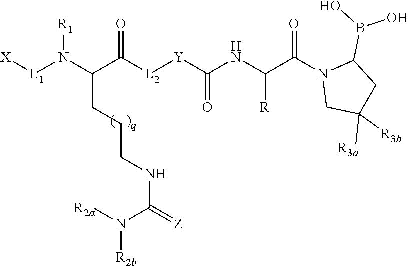
wherein:
X is a chelator suitable for radiolabeling, or a chelator bound to a radioactive atom or complex;
L₁ is a bond, or a group of the formula: 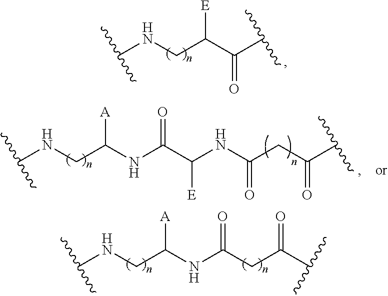
wherein each n is independently 0, 1, 2, 3, 4, or 5;
q is 0, 1, or 2;
Z is ═O or ═NR_{2c};
A is —H or —COOH;
E is —H, 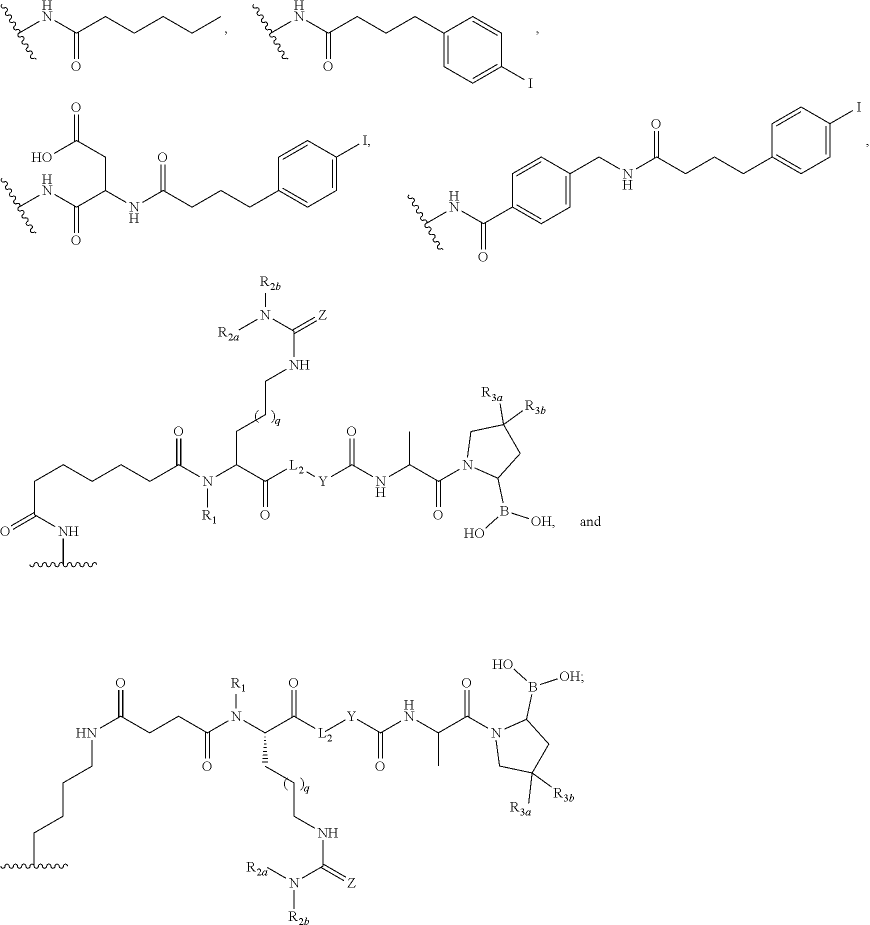
wherein
each R is independently —H, —CH₃, or —CH₂OH;
R₁ is —H or —C₁₋₃ alkyl;
R₂ₐ, R_{2b} and R_{2c} are each independently —H or —C₁₋₃ alkyl;
R₃ₐ and R_{3b} are each independently —H, —OH, or halogen; and
L₂ is a group of the formula: 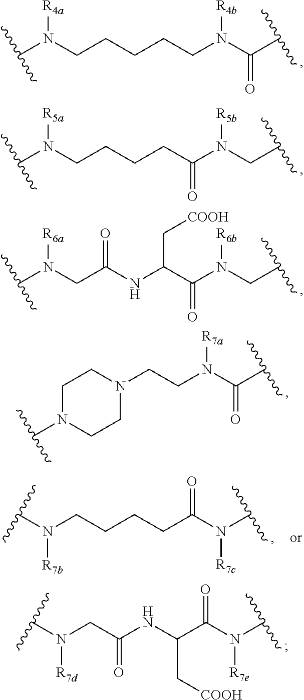
wherein R₄ₐ and R_{4b} are each independently —H or —C₁₋₃ alkyl;
R₅ₐ and R_{5b} are each independently —H or —C₁₋₃ alkyl;
R₆ₐ and R_{6b} are each independently —H or —C₁₋₃ alkyl;
R₇ₐ is —OH or —C₁₋₃ alkyl;
R_{7b} and R_{7c} are each independently —H or —C₁₋₃ alkyl; and
R_{7d} and R₇ₑ are each independently —H or —C₁₋₃ alkyl;
Y is selected from the group consisting of: 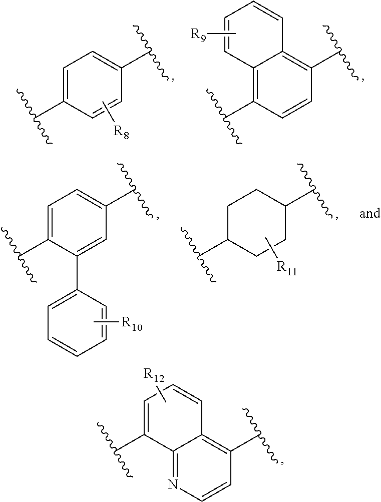
wherein R₈ is —H, —OH, halogen, —OR₈ₐ, —NR_{8b}R_{8c}, —COOH, or a 5 to 6 membered heteroaryl;
R₉ is —H, —OH, halogen, —OR₉ₐ, —NR_{9b}R_{9c}, or —COOH;
R₁₀ is —H, —OH, halogen, —OR₁₀ₐ, —NR_{10b}R_{10c}, or —COOH; and
R₁₁ is —H, —OH, halogen, —OR₁₁ₐ, —NR_{11b}R_{11c}, or —COOH;
wherein R₈ₐ, R_{8b}, and R_{8c} are each independently —H or —C₁₋₃ alkyl;
R₉ₐ, R_{9b}, and R_{9c} are each independently —H or —C₁₋₃ alkyl;
R₁₀ₐ, R_{10b}, and R_{10c} are each independently —H or —C₁₋₃ alkyl;
R₁₁ₐ, R_{11b}, and R_{11c} are each independently —H or —C₁₋₃ alkyl; and
R₁₂ is —H, —OH, halogen, —OR₉ₐ, —NR_{9b}R_{9c}, or —COOH;
or a pharmaceutically acceptable salt thereof.

2. The compound according to claim 1 wherein L₂ is a group of the formula: 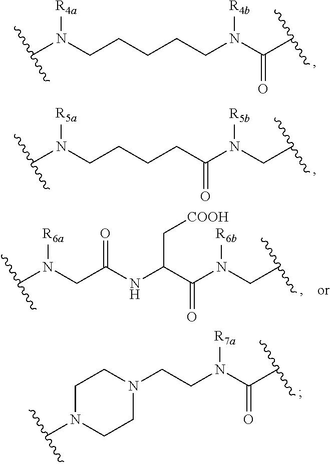
wherein R₄ₐ and R_{4b} are each independently —H or —C₁₋₃ alkyl;
R₅ₐ and R_{4b} are each independently —H or —C₁₋₃ alkyl;
R₆ₐ and R_{6b} are each independently —H or —C₁₋₃ alkyl; and
R₇ₐ is —OH or —C₁₋₃ alkyl.

3. The compound according to claim 1 of the formula: 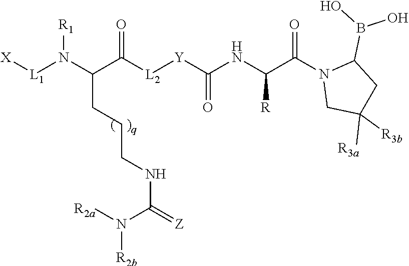
or a pharmaceutically acceptable salt thereof.

4. The compound according to claim 1 of the formula: 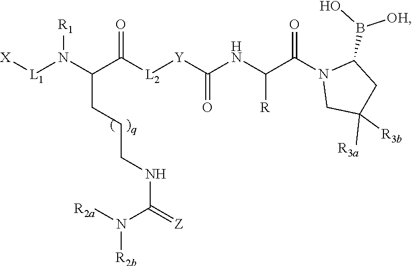
or a pharmaceutically acceptable salt thereof.

5. The compound according to claim 1 of the formula: 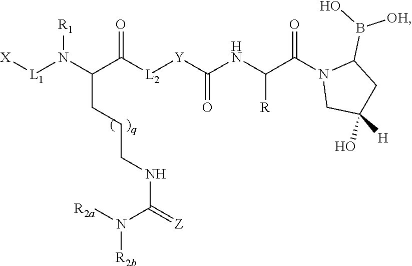
or a pharmaceutically acceptable salt thereof.

6. The compound according to claim 1 of the formula: 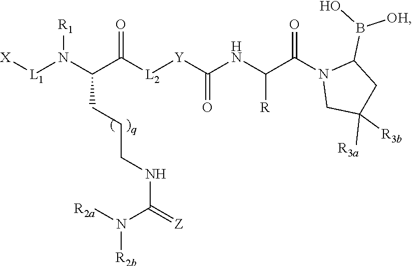
or a pharmaceutically acceptable salt thereof.

7. The compound according to claim 1, or a pharmaceutically acceptable salt thereof, wherein each R is methyl.

8. The compound according to claim 1, or a pharmaceutically acceptable salt thereof, wherein L₁ is selected from the group consisting of: a bond, or a group of the formula: 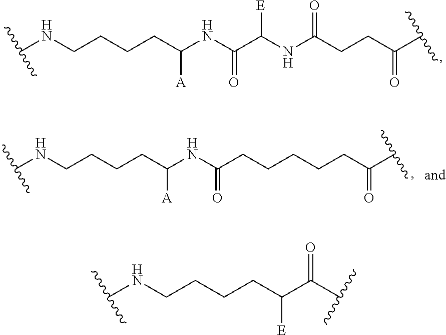
wherein A is —H or —COOH.

9. The compound according to claim 1, or a pharmaceutically acceptable salt thereof, wherein L₁ is selected from the group consisting of: a bond, or a group of the formula:

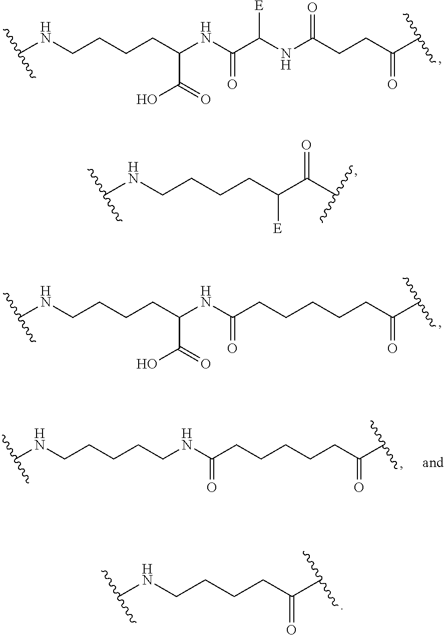

10. The compound according to claim 1, or a pharmaceutically acceptable salt thereof, wherein E is selected from the group consisting of:

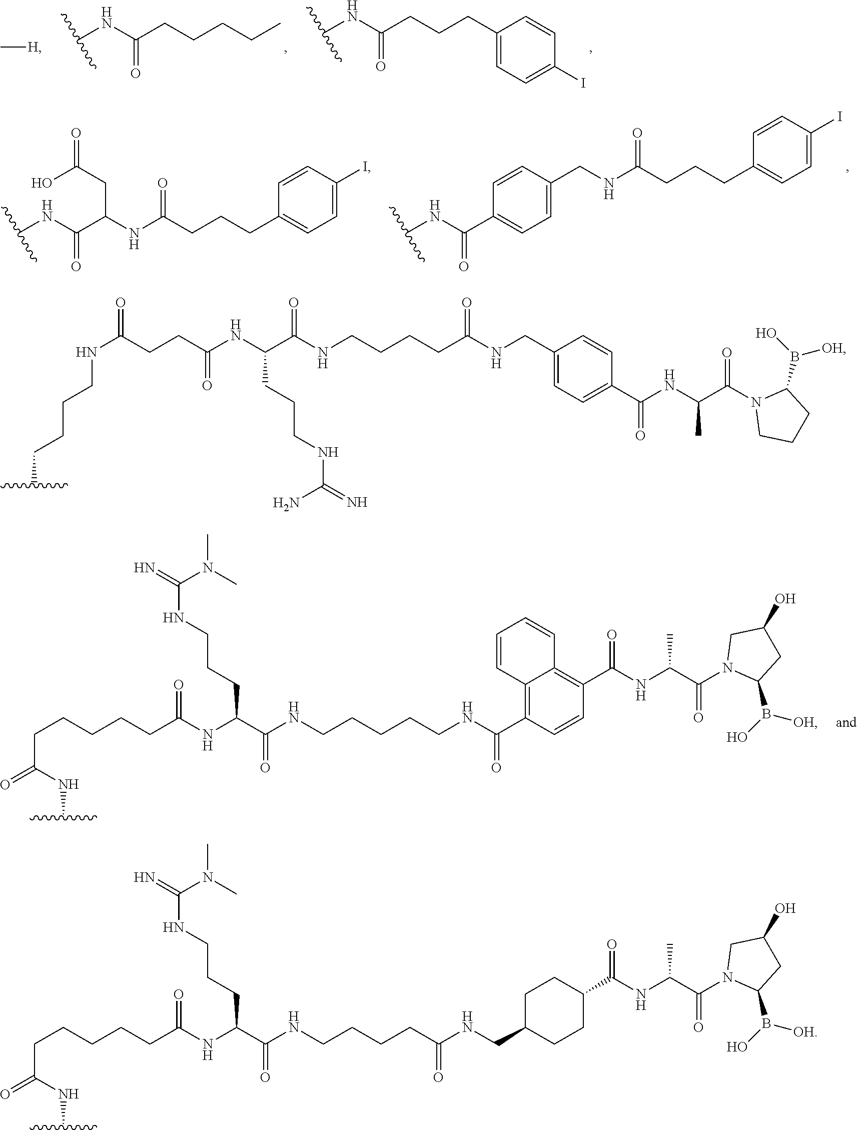

11. The compound according to claim 1, or a pharmaceutically acceptable salt thereof, wherein L₂ is selected from the group consisting of:

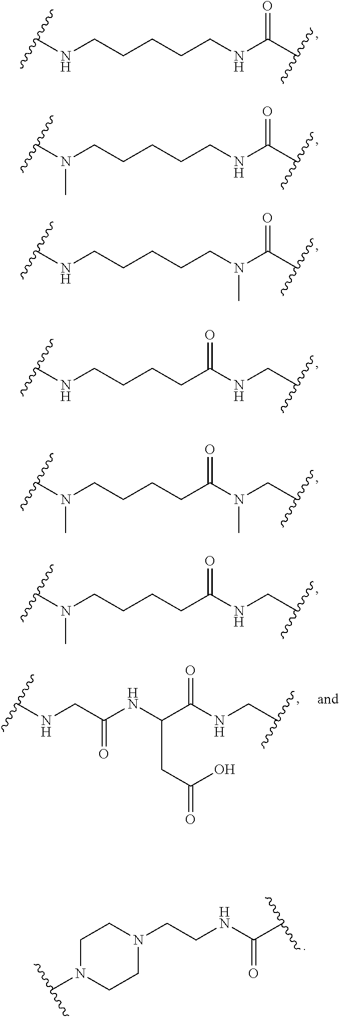

12. (canceled)

13. The compound according to claim 1, or a pharmaceutically acceptable salt thereof, wherein
R₈ is —H, —F, —OCH₃, —COOH, or 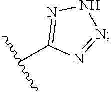 [SMILES: *c1nn[nH]n1]
R₉ is —H, —OCH₃, or —N(CH₃)₂; and
R₁₀ is —H, —Cl, —OH, or —N(CH₃)₂.

14. The compound according to claim 1, or a pharmaceutically acceptable salt thereof, wherein Y is selected from the group consisting of:

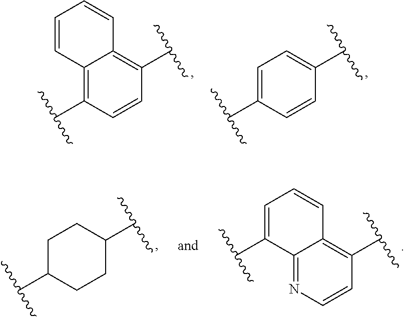 [SMILES: *c1ccc(*)c2ccccc12 ; *c1ccc(*)cc1 ; *C1CCC(*)CC1 ; *c1ccnc2c(*)cccc12]

15. The compound according to claim 1, or a pharmaceutically acceptable salt thereof, wherein X is a chelator suitable for radiolabeling selected from the group consisting of: 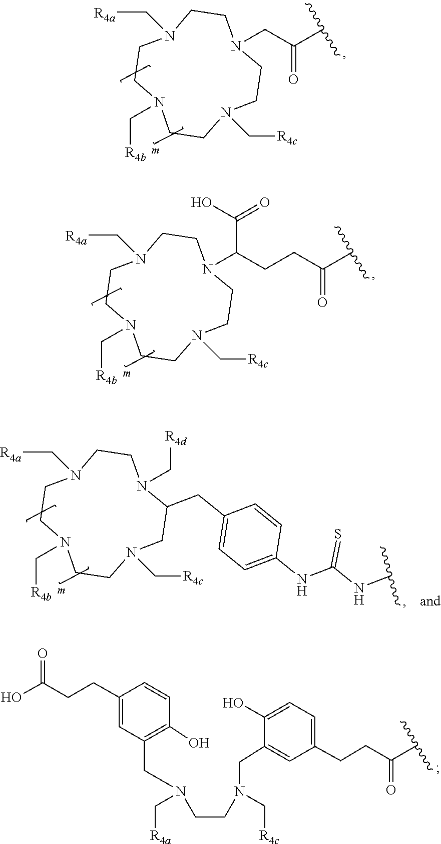
m is 0, 1, or 2;
R₄ₐ is independently —H, —COOH, or —CONH₂;
R_{4b} is independently —H, —COOH, or —CONH₂; and
R_{4c} is independently —H, —COOH, or —CONH₂.

16-19. (canceled)

20. The compound according to claim 1, or a pharmaceutically acceptable salt thereof, wherein X is a chelator bound to a radioactive atom and the radioactive atom is selected from the group consisting of: ²²³Ra, ⁸⁹Sr, ^{94m}Tc ^{99m}Tc, ¹⁸⁶Re, ¹⁸⁸Re, ²⁰³Pb, ²¹²Pb, ⁶⁷Ga, ⁶⁸Ga, ⁴⁷Sc, ¹¹¹In, ⁹⁷Ru, ⁶²Cu, ⁶⁴Cu, ⁶⁷Cu, ⁸⁶Y, ⁸⁸Y, ⁸⁹Zr, ⁹⁰Y, ¹²¹Sn, ¹⁶¹Tb, ¹⁵³Sm, ¹⁶⁶Ho, ¹⁰⁵Rh, ¹⁷⁷Lu, ¹²³I, ¹²⁴I, ¹²⁵I, ¹³¹I, ¹⁸F, ²¹¹At, ²²⁵Ac, ⁸⁹Sr, ^{117m}Sn, ¹⁶⁹Er, and [¹⁸F]AlF.

21. The compound according to claim 1, or a pharmaceutically acceptable salt thereof, wherein X is a chelator bound to a radioactive atom and the radioactive atom is selected from the group consisting of: ²²⁵Ac, ²¹¹At, ⁶⁴Cu, ⁶⁷Cu, ¹⁸F, [¹⁸F]AlF, ⁶⁷Ga, ⁶⁸Ga, ¹¹¹In, ¹⁷⁷Lu, ²⁰³Pb ²¹²Pb, ¹⁶¹Tb, ⁸⁶Y, ⁹⁰Y, and ⁸⁹Zr.

22. The compound according to claim 21, or a pharmaceutically acceptable salt thereof, wherein the radioactive atom is ¹⁷⁷Lu.

23. The compound according to claim 21, or a pharmaceutically acceptable salt thereof, wherein the radioactive atom is ²¹²Pb.

24. The compound according to claim 21, or a pharmaceutically acceptable salt thereof, wherein the radioactive atom is [¹⁸F]AlF.

25. The compound according to claim 21, or a pharmaceutically acceptable salt thereof, wherein the radioactive atom is ²²⁵Ac.

26. (canceled)

27. The compound according to claim 1, or a pharmaceutically acceptable salt thereof, wherein X is a chelator bound to a radioactive atom selected from the group consisting of: 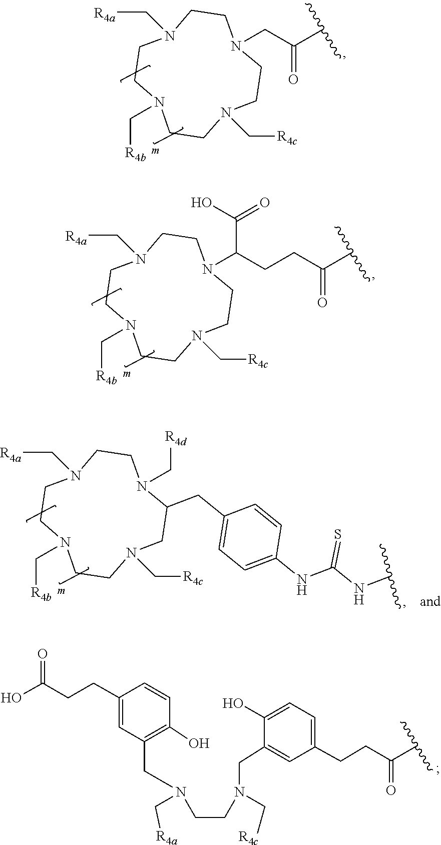
wherein M is the radioactive atom,
m is 0, 1, or 2;
R₄ₐ is independently —H, —COOH, or —CONH₂;
R_{4b} is independently —H, —COOH, or —CONH₂;
R_{4c} is independently —H, —COOH, or —CONH₂; and
R₄ₐ is independently —H, —COOH, or —CONH₂.

28-30. (canceled)

31. The compound according to claim 1, or a pharmaceutically acceptable salt thereof, selected from the group consisting of: 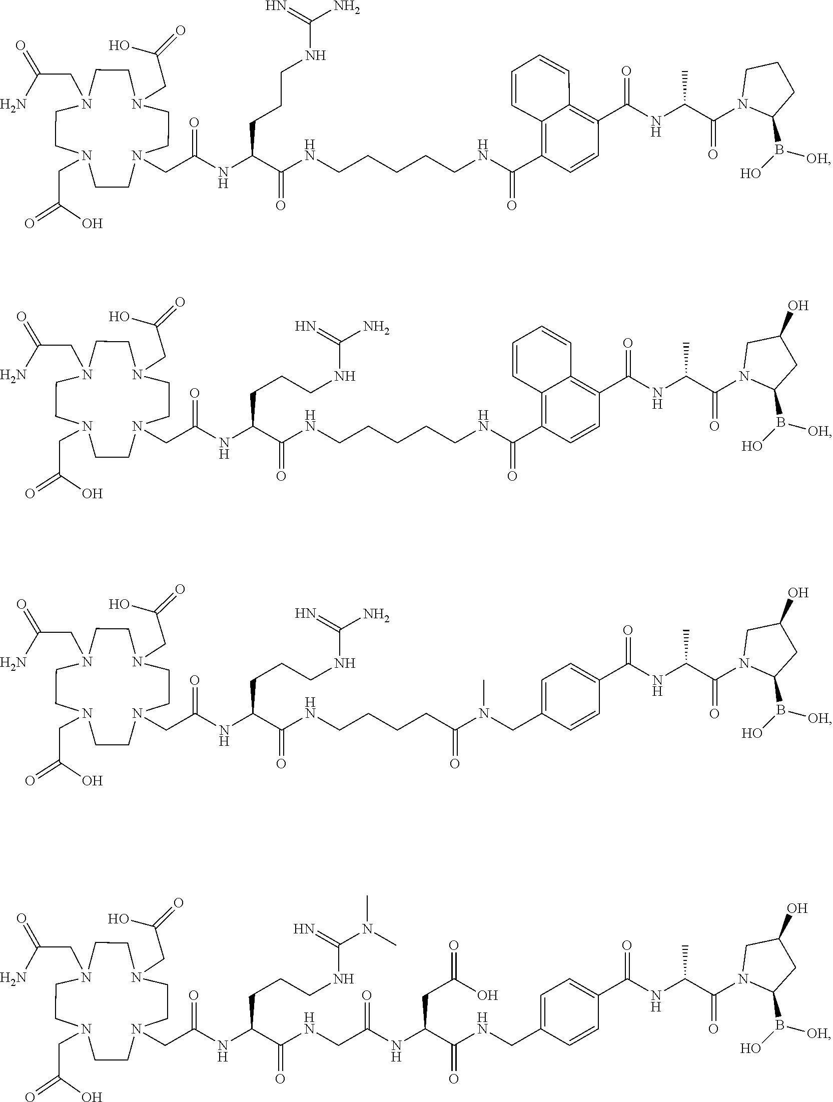 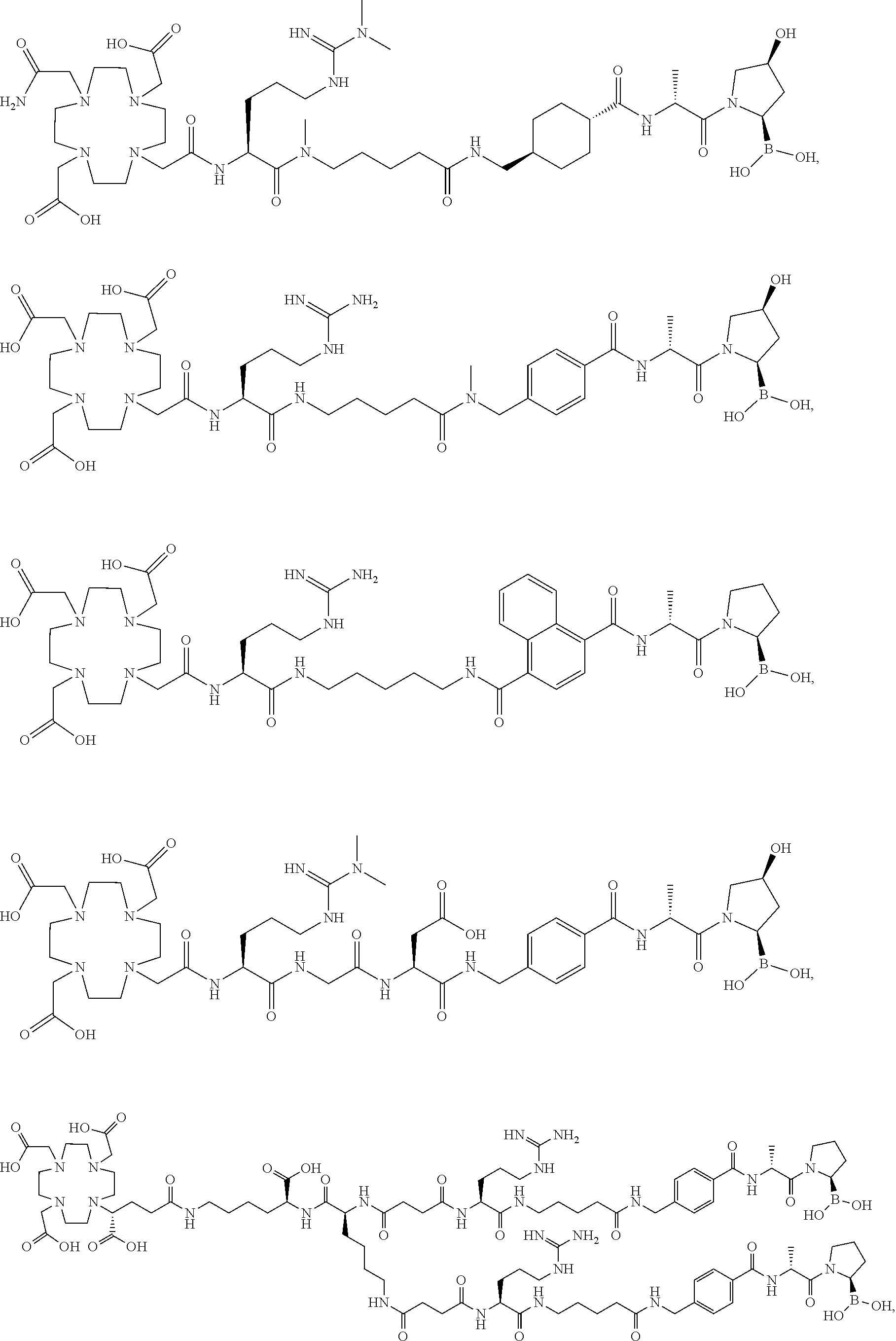 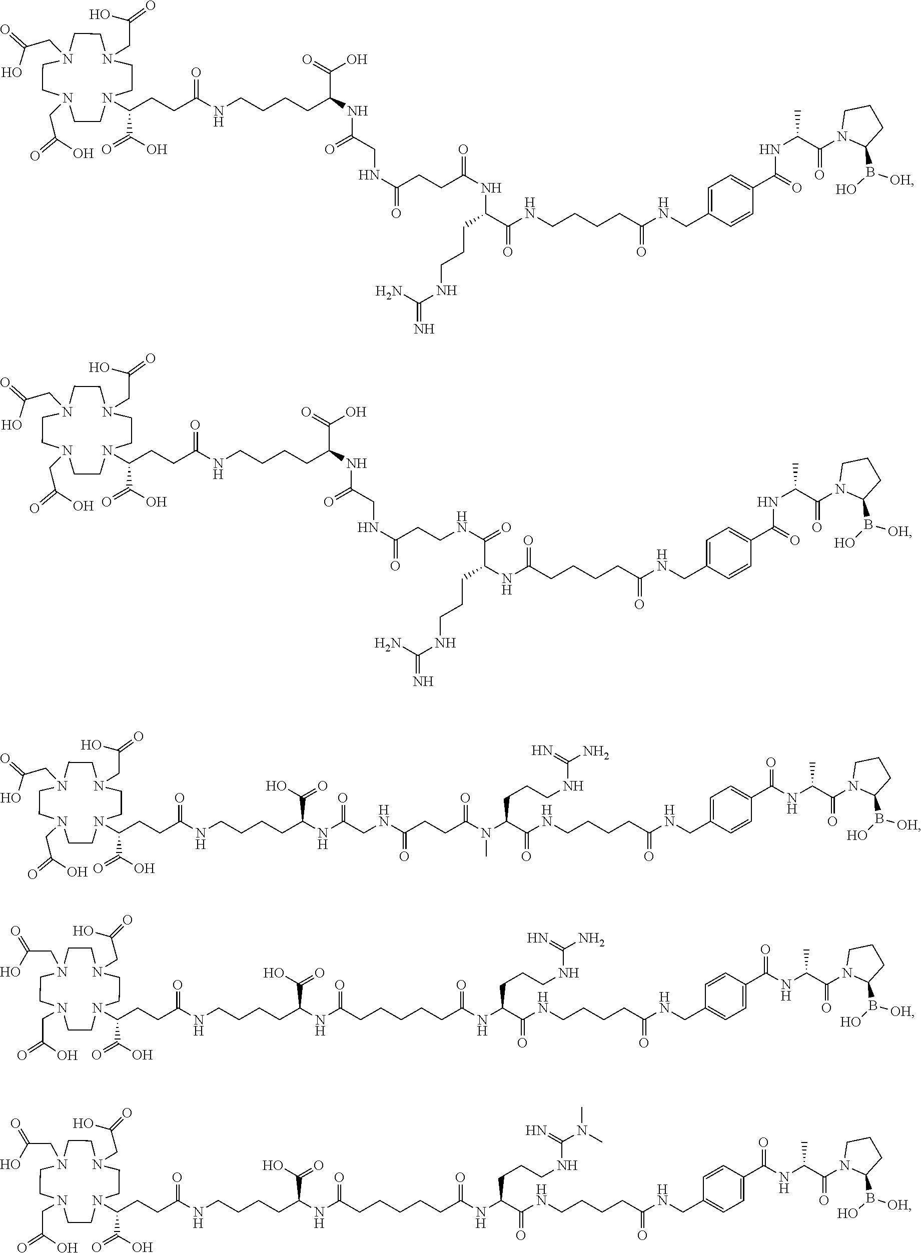 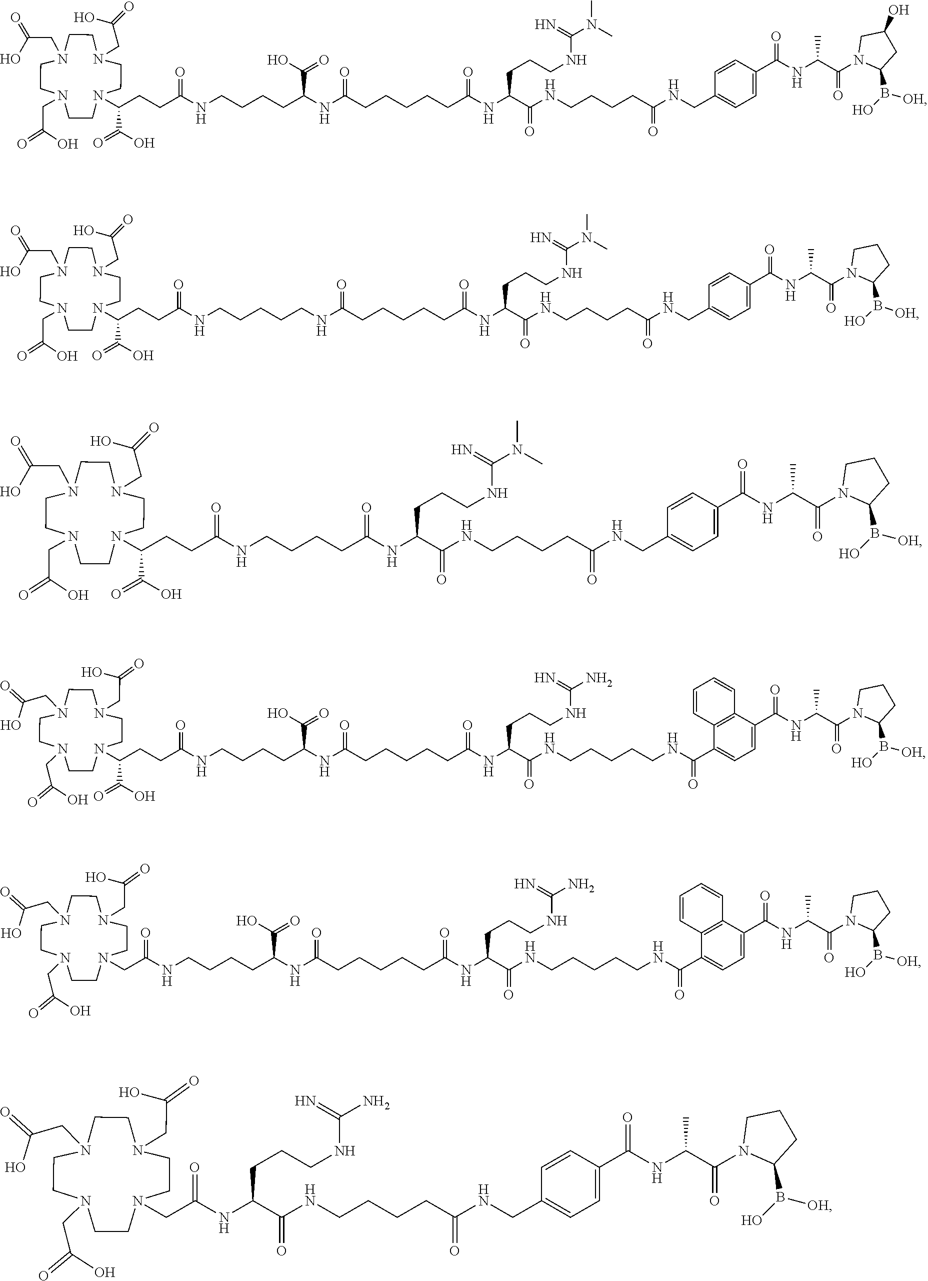 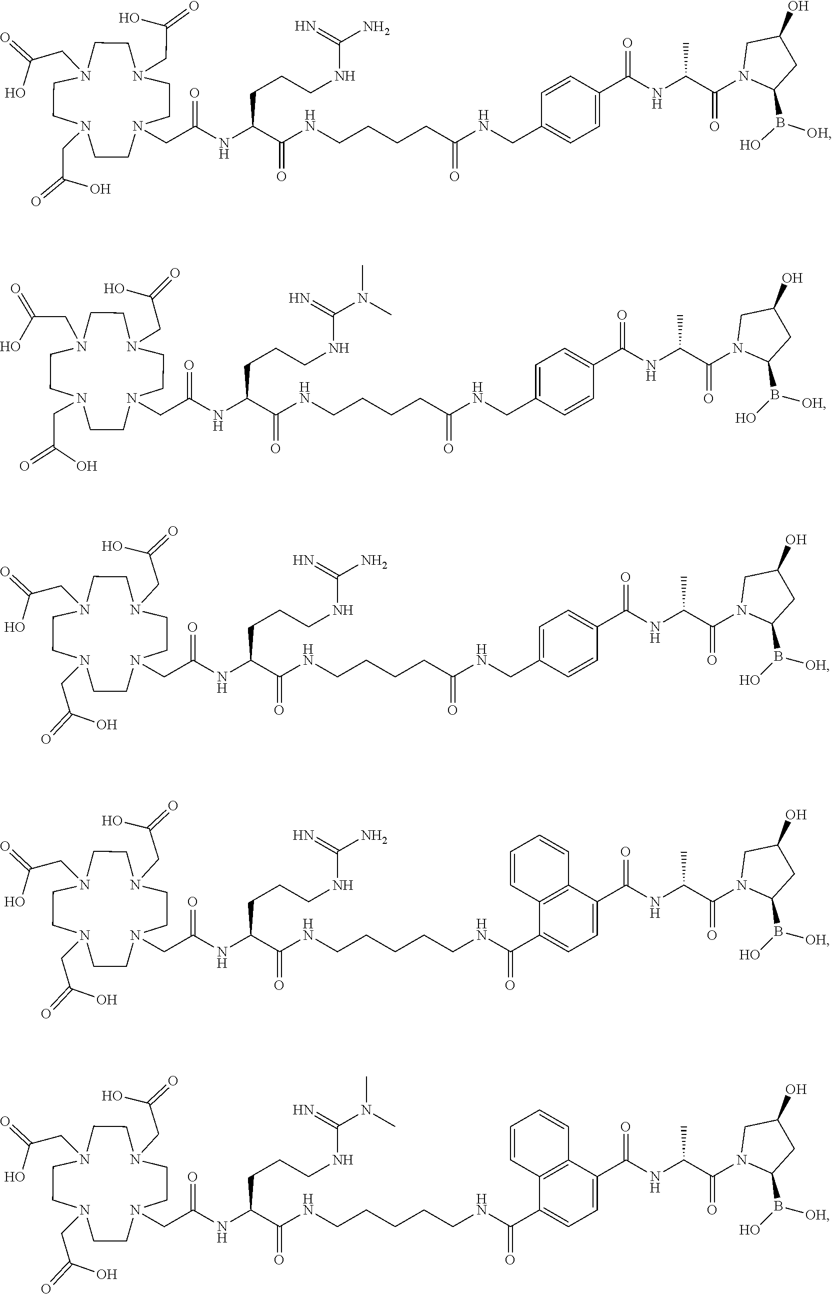 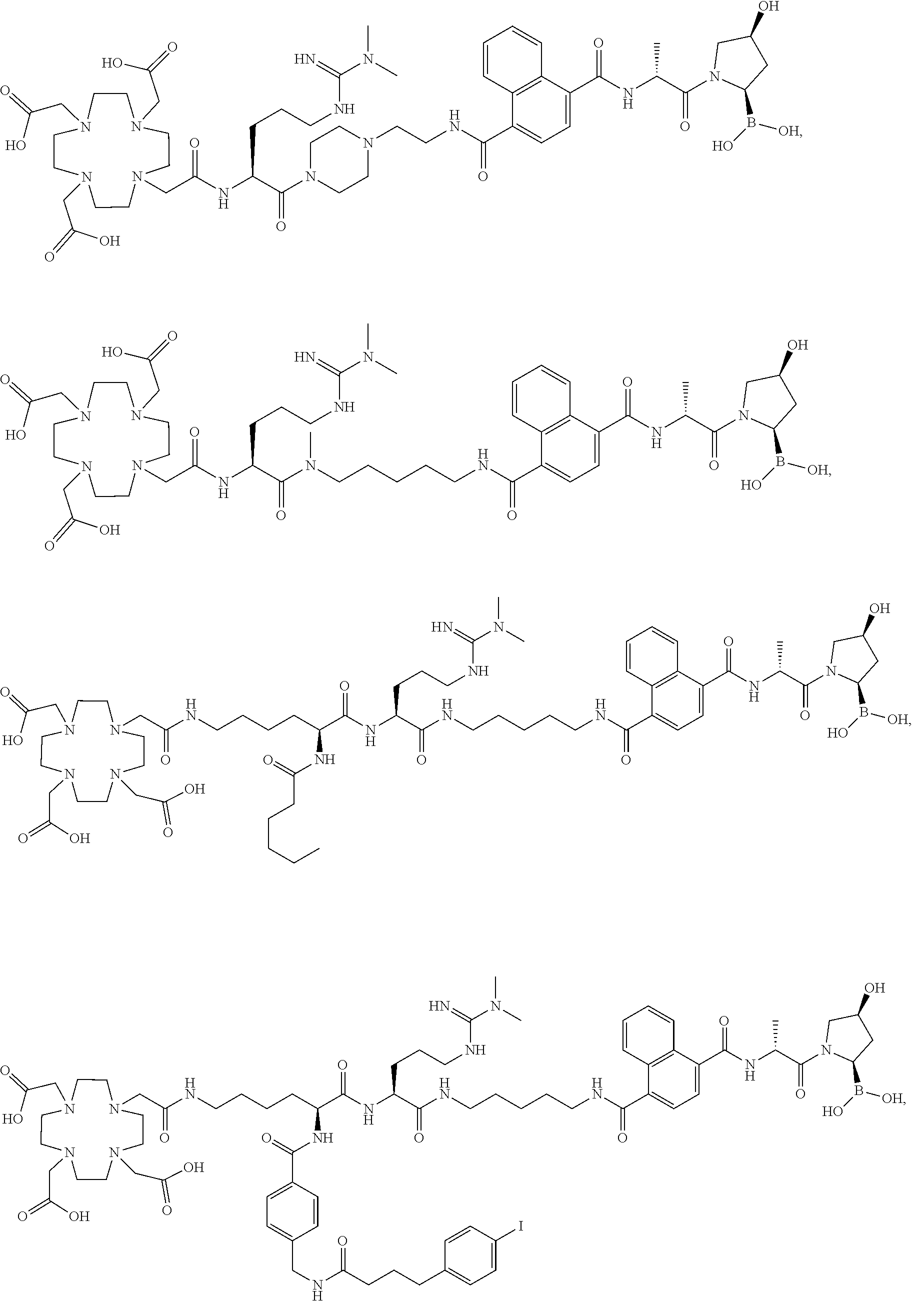 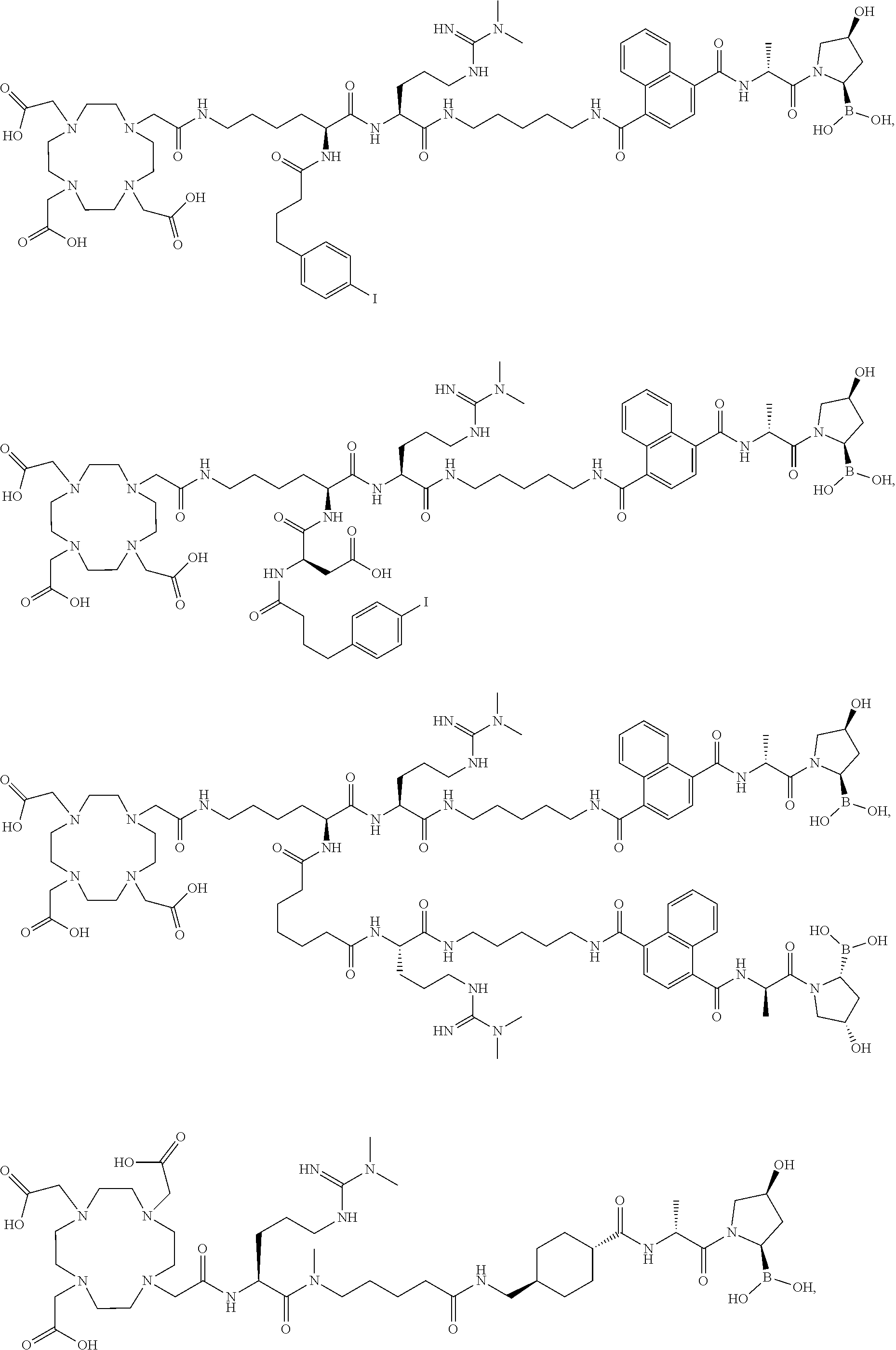 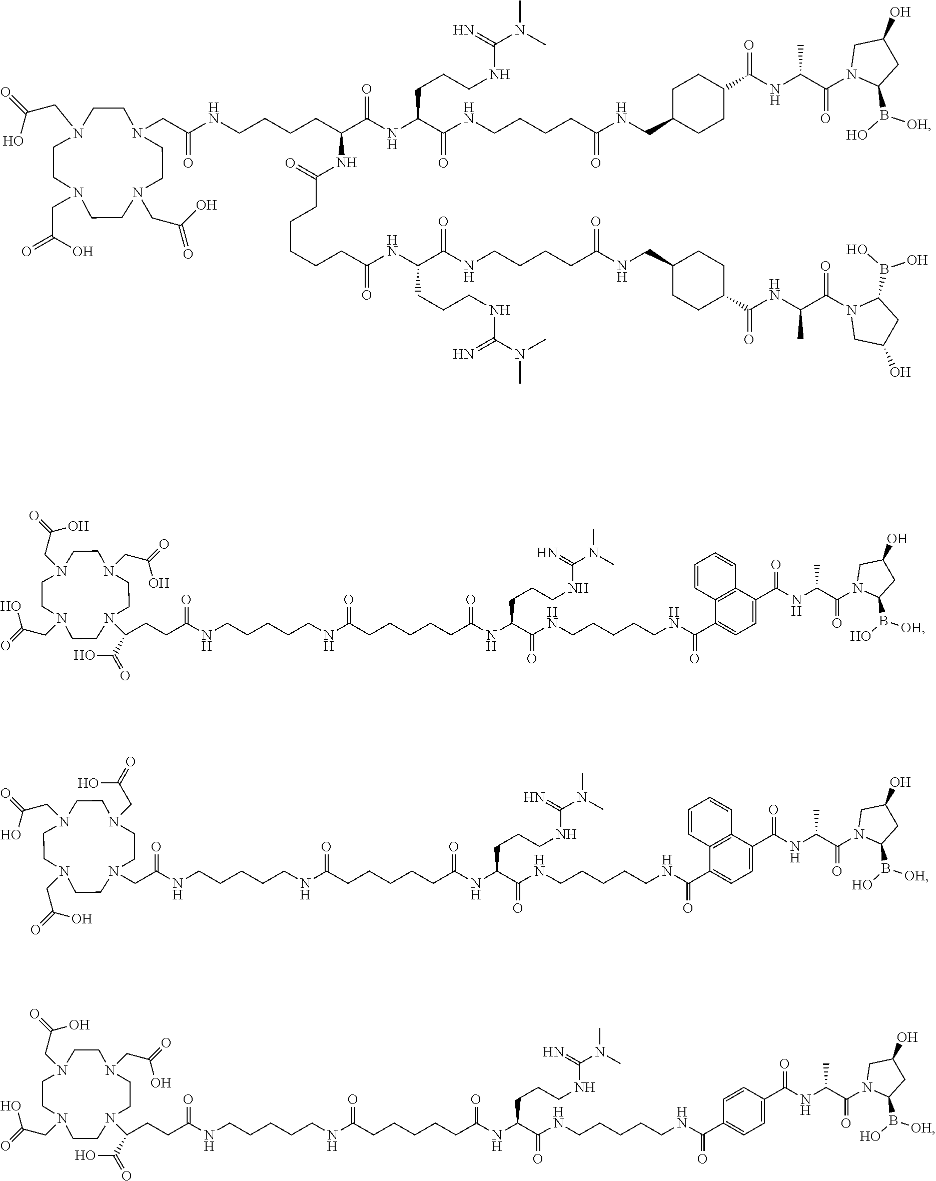 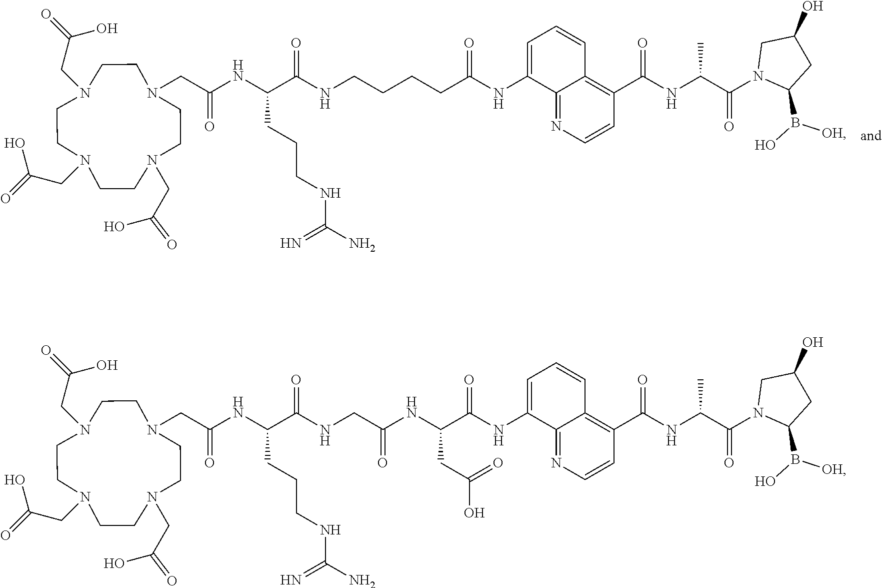 [SMILES: C[C@@H](NC(=O)c1ccnc2c(NC(=O)CCCCNC(=O)[C@H](CCCNC(=N)N)NC(=O)CN3CCN(CC(=O)O)CCN(CC(=O)O)CCN(CC(=O)O)CC3)cccc12)C(=O)N1C[C@@H](O)C[C@H]1B(O)O ; C[C@@H](NC(=O)c1ccnc2c(NC(=O)[C@H](CC(=O)O)NC(=O)CNC(=O)[C@H](CCCNC(=N)N)NC(=O)CN3CCN(CC(=O)O)CCN(CC(=O)O)CCN(CC(=O)O)CC3)cccc12)C(=O)N1C[C@@H](O)C[C@H]1B(O)O]
or a pharmaceutically acceptable salt thereof.

32. The compound according to claim 1, or a pharmaceutically acceptable salt thereof, selected from the group consisting of:

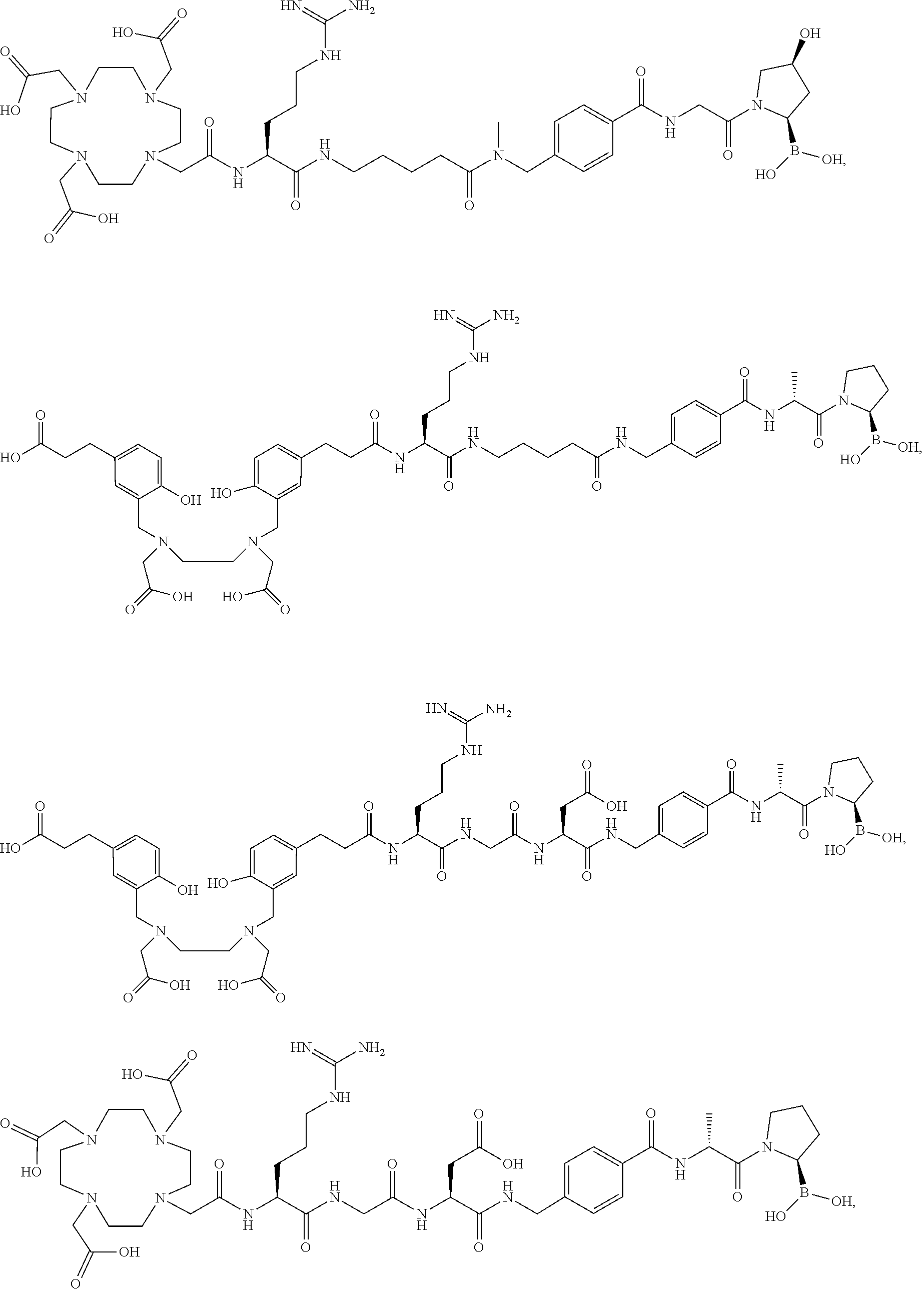

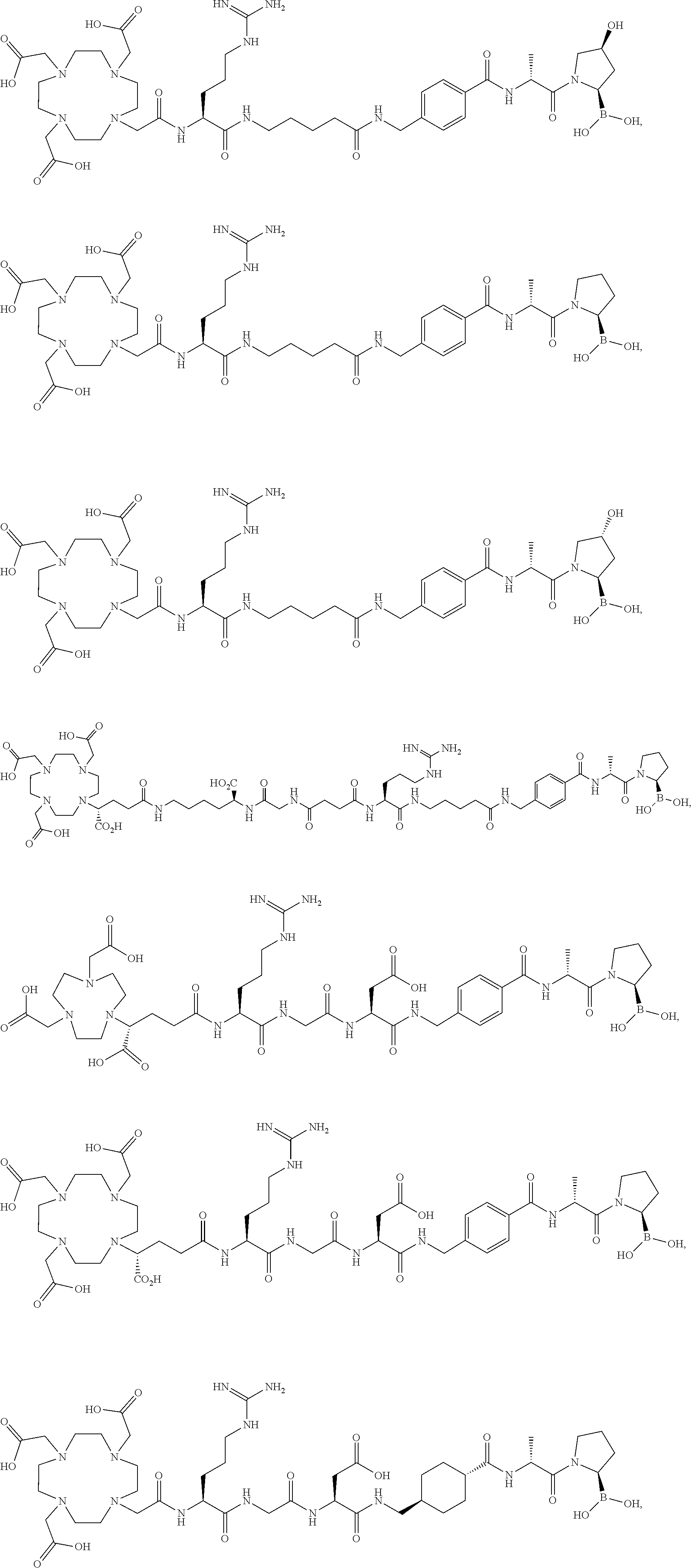

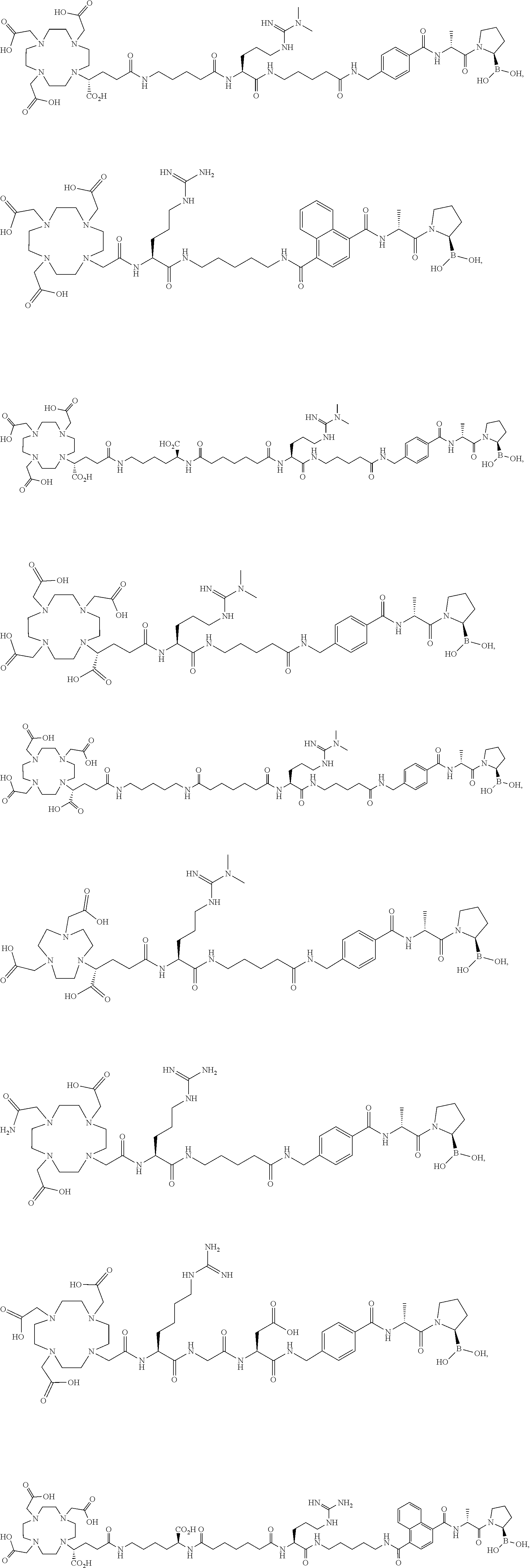

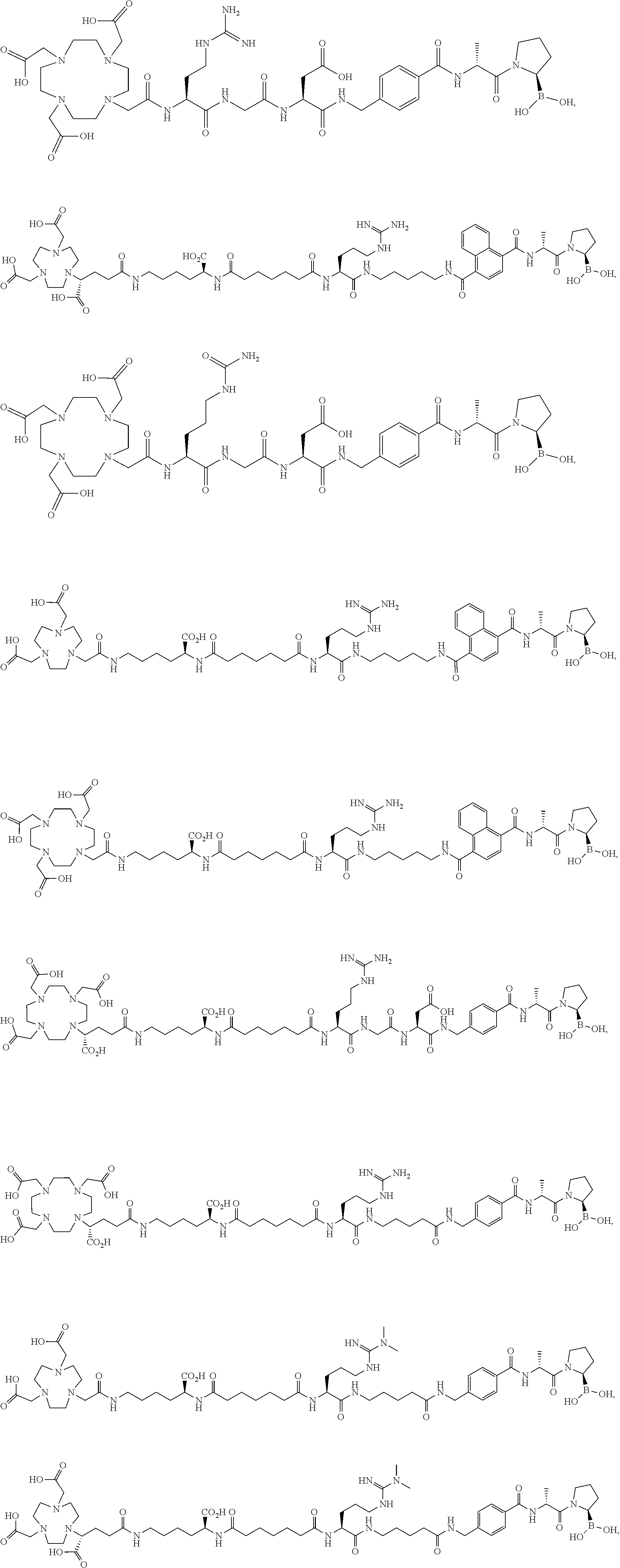

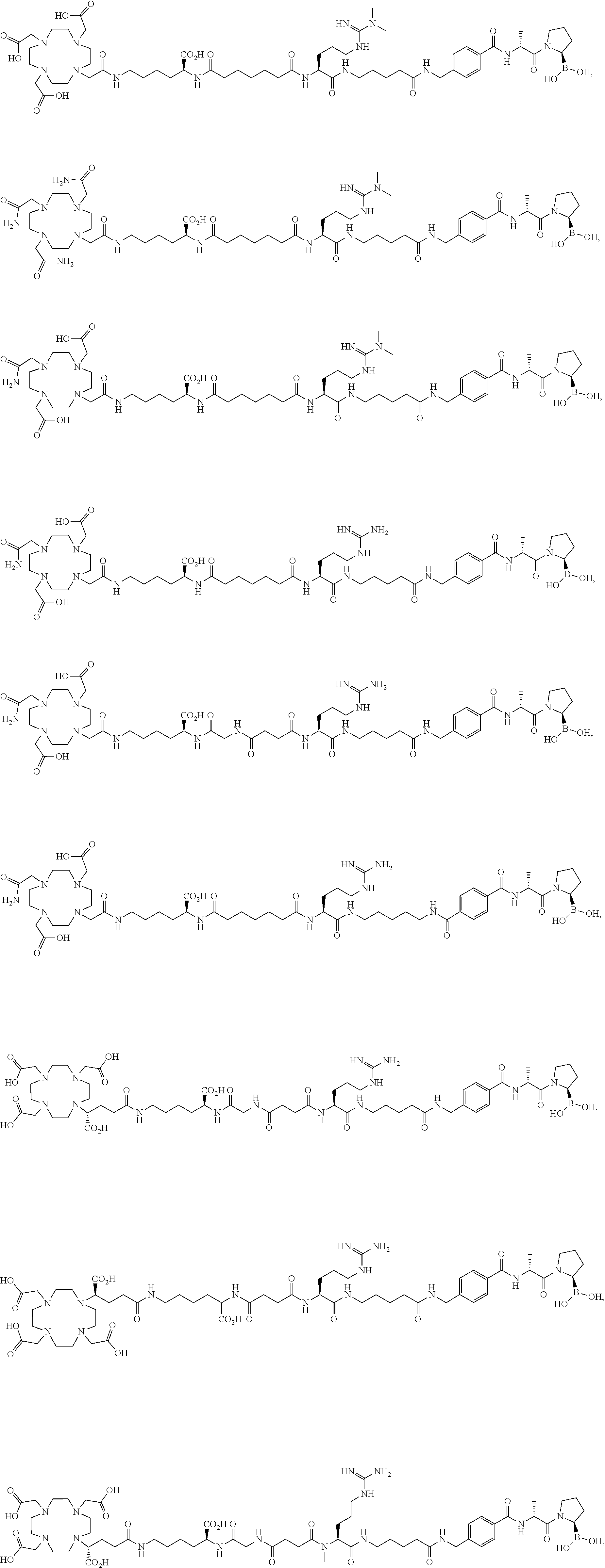

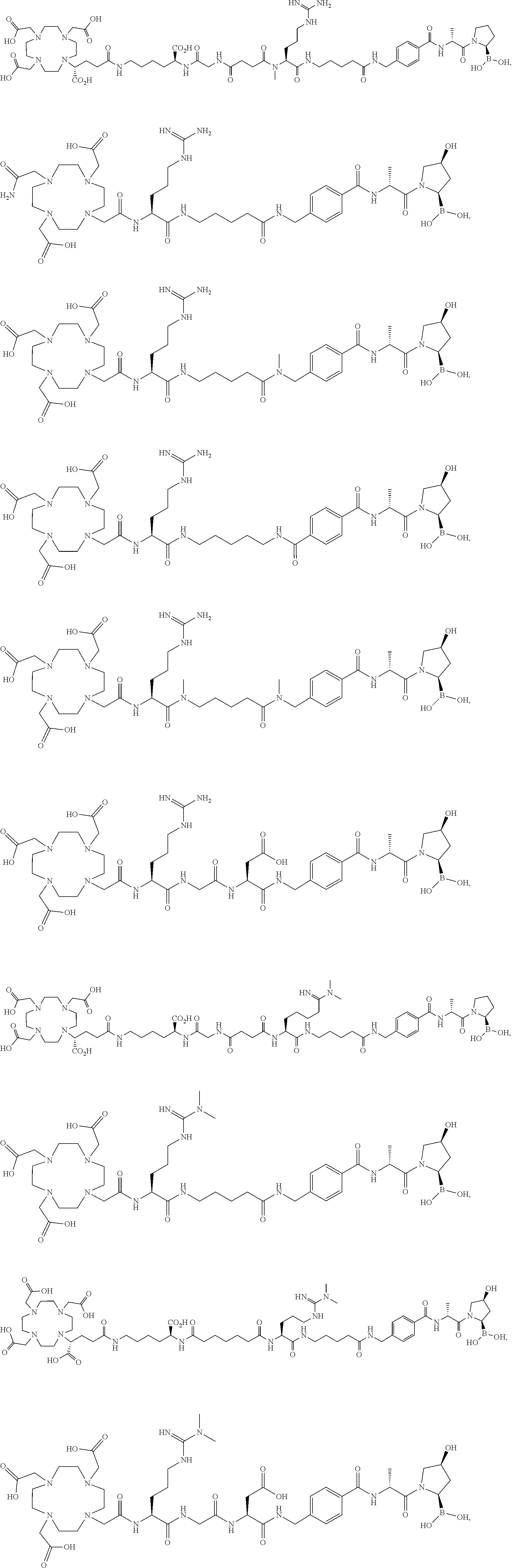

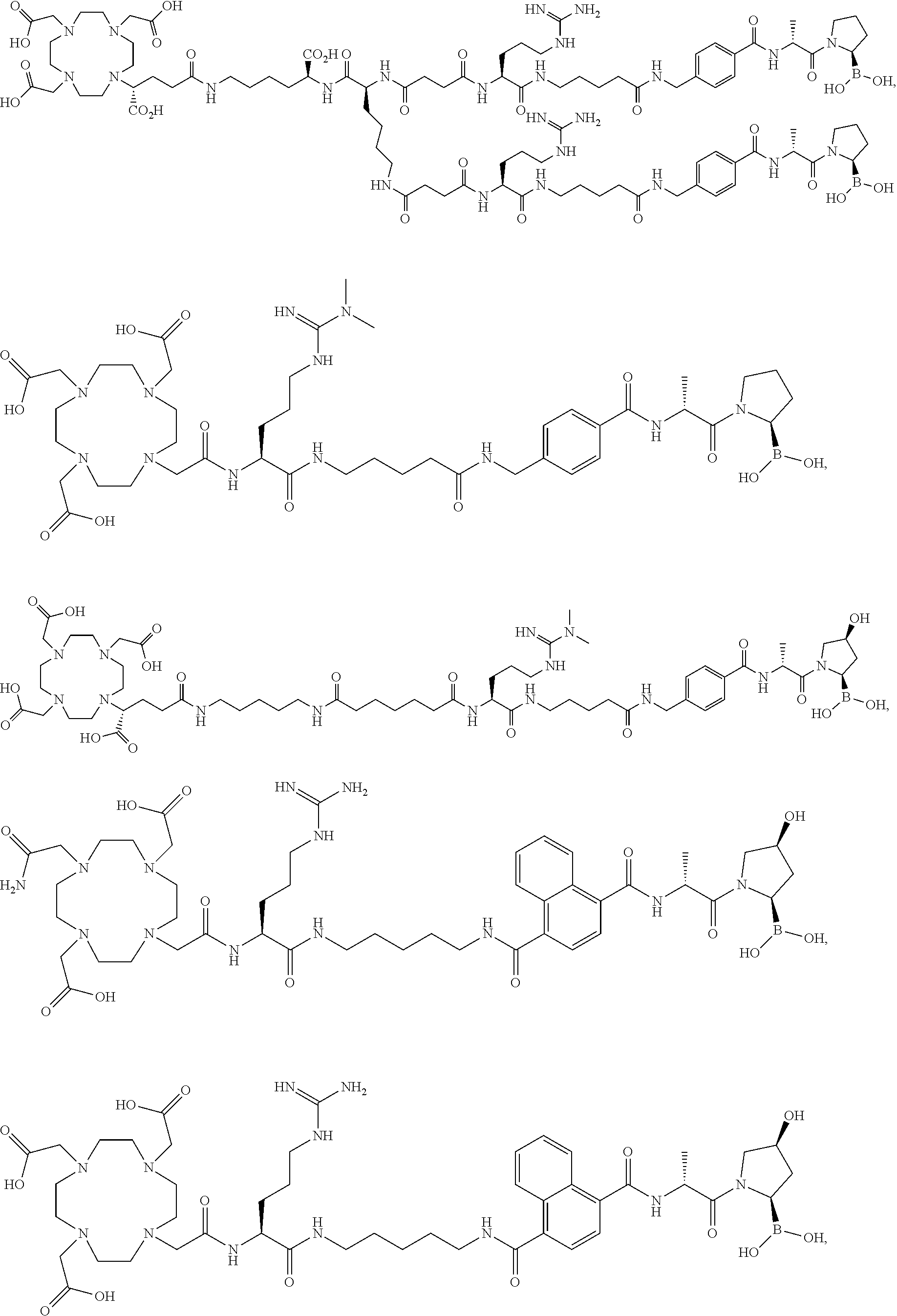

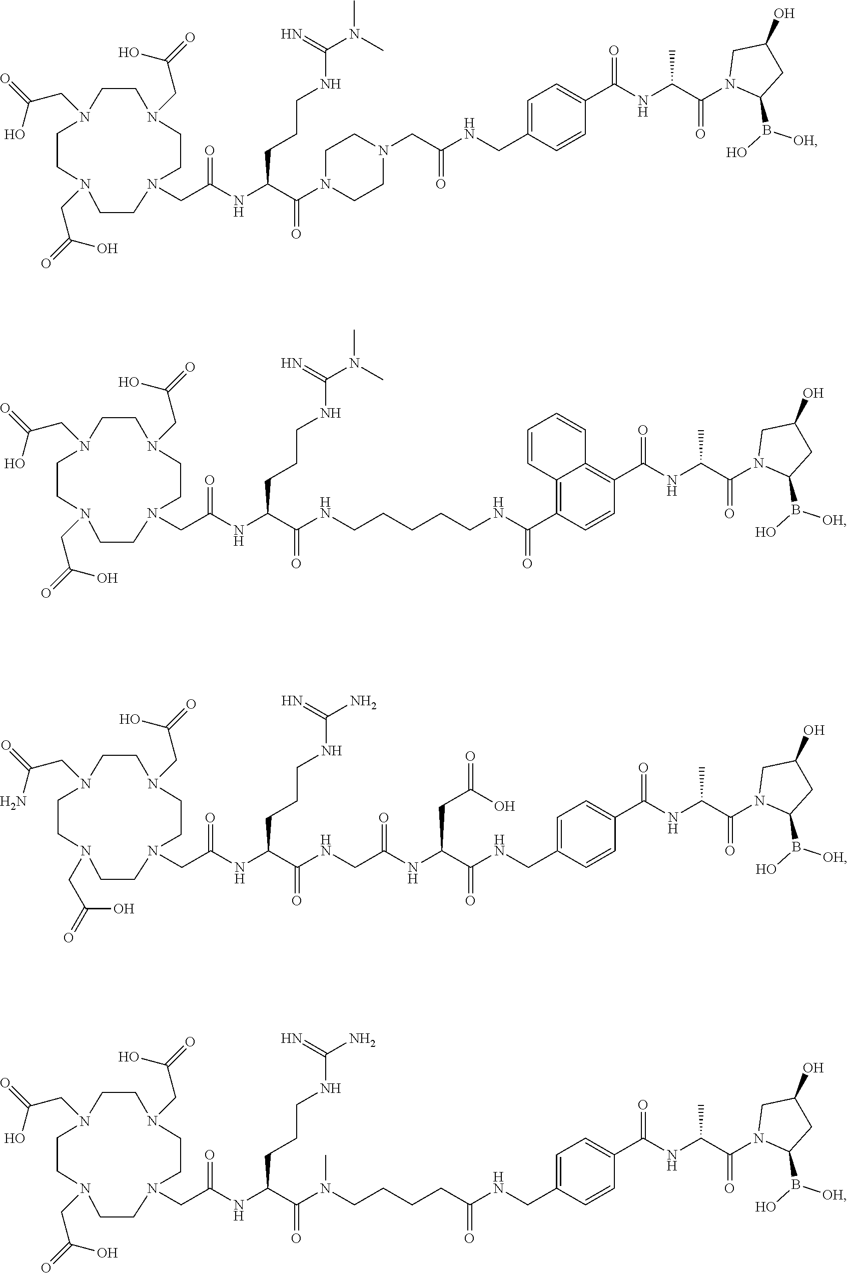

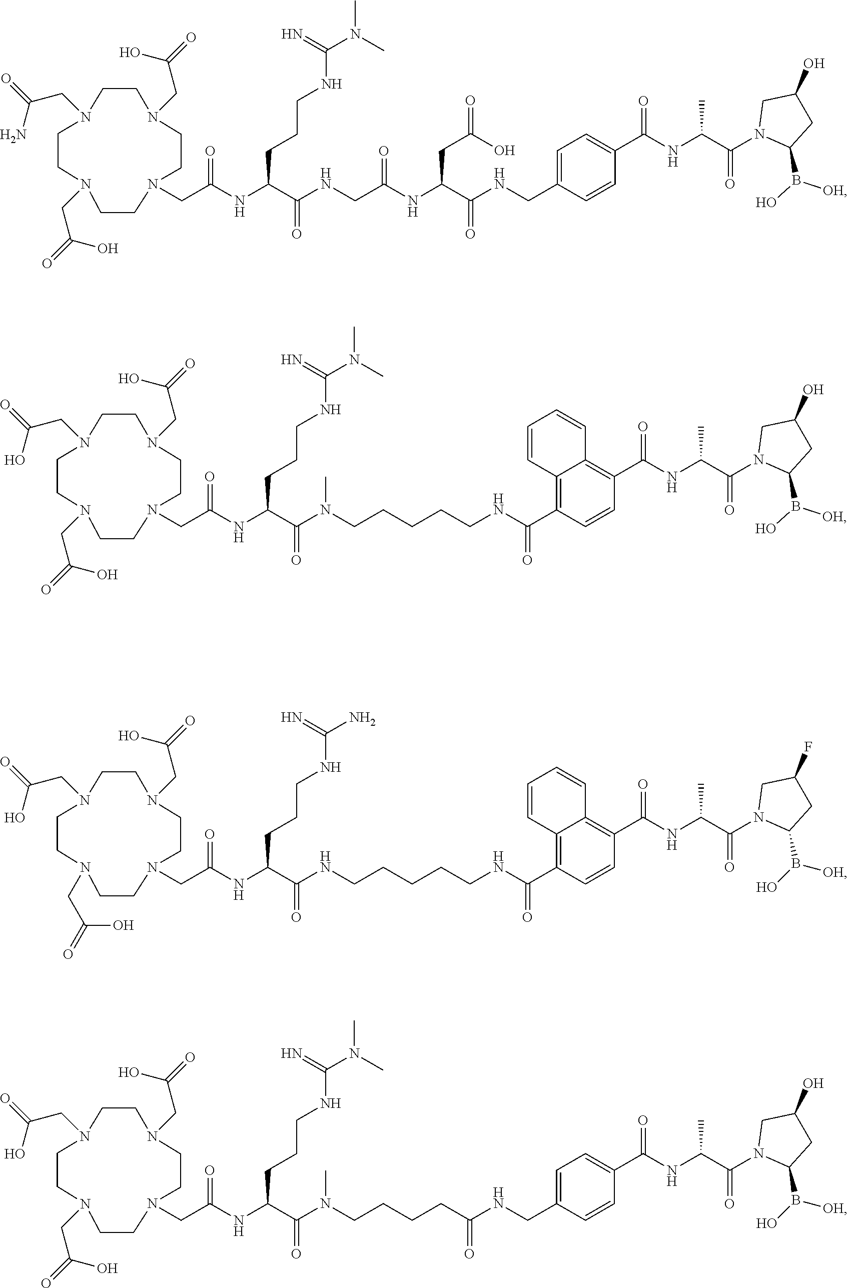

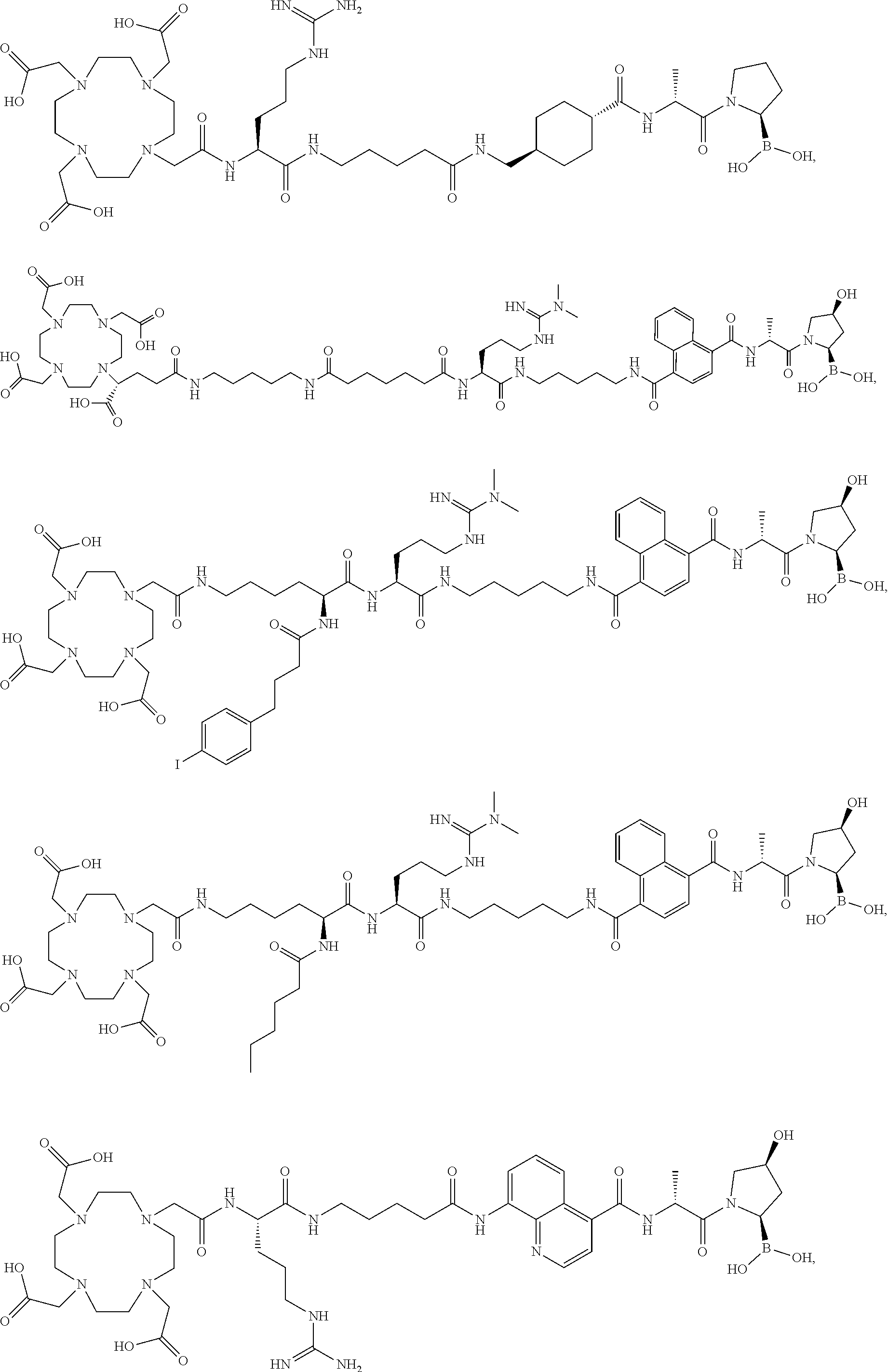

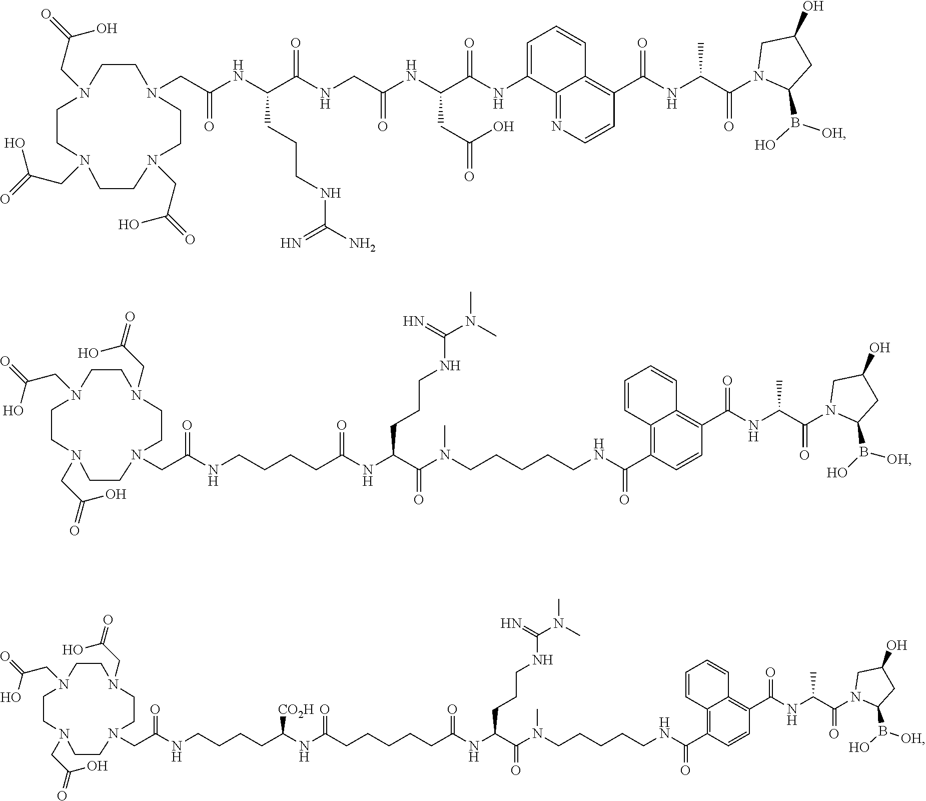

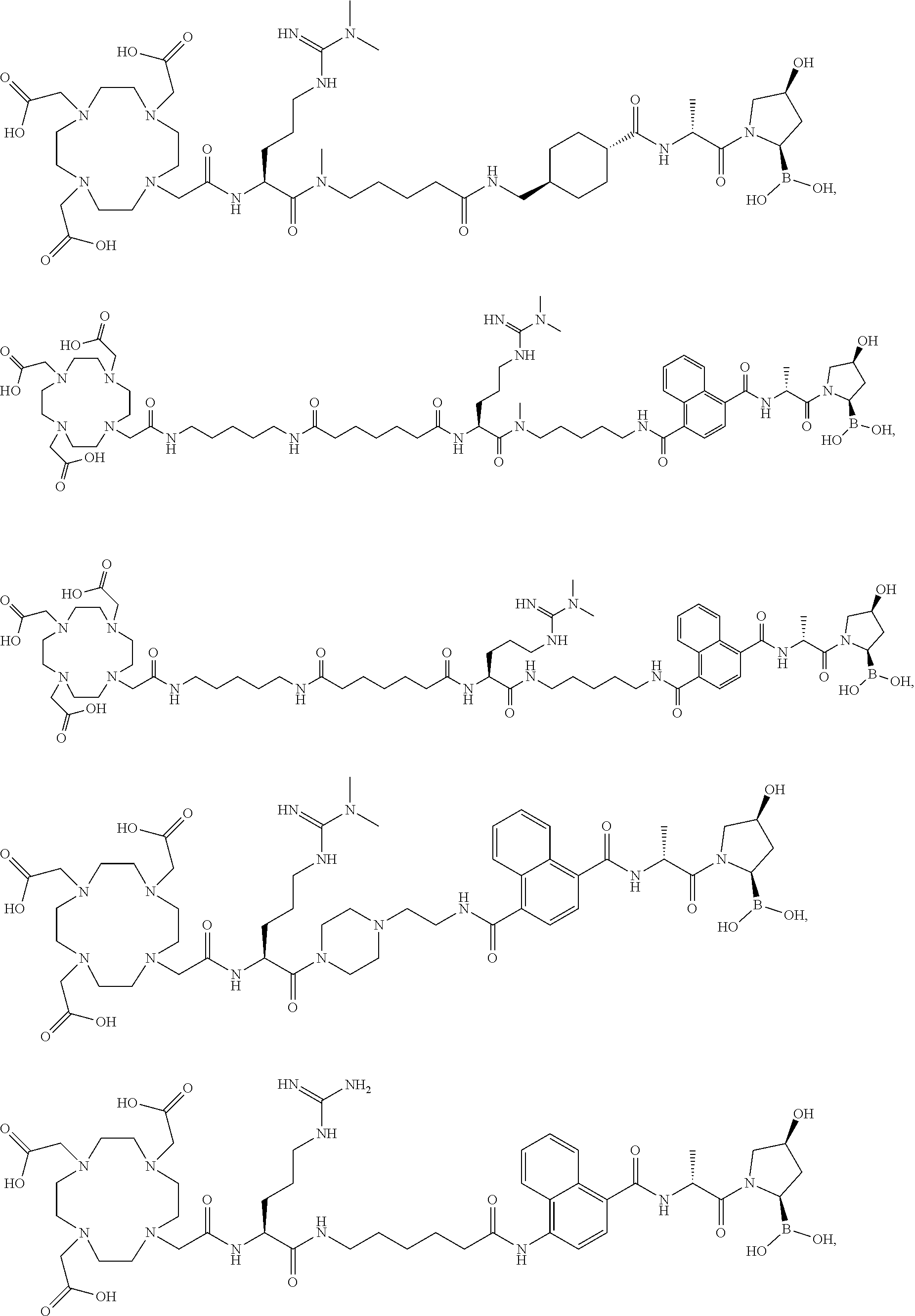

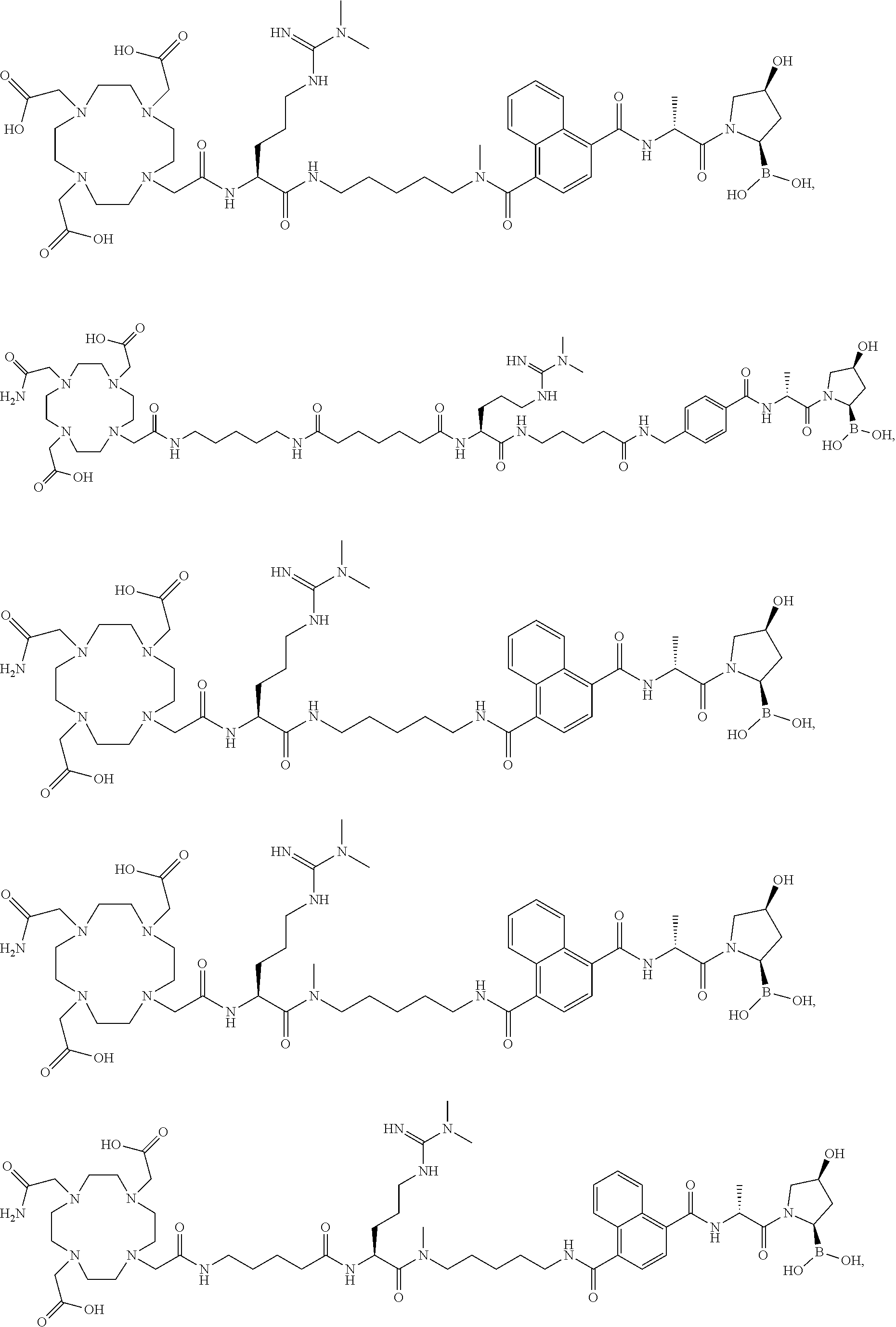

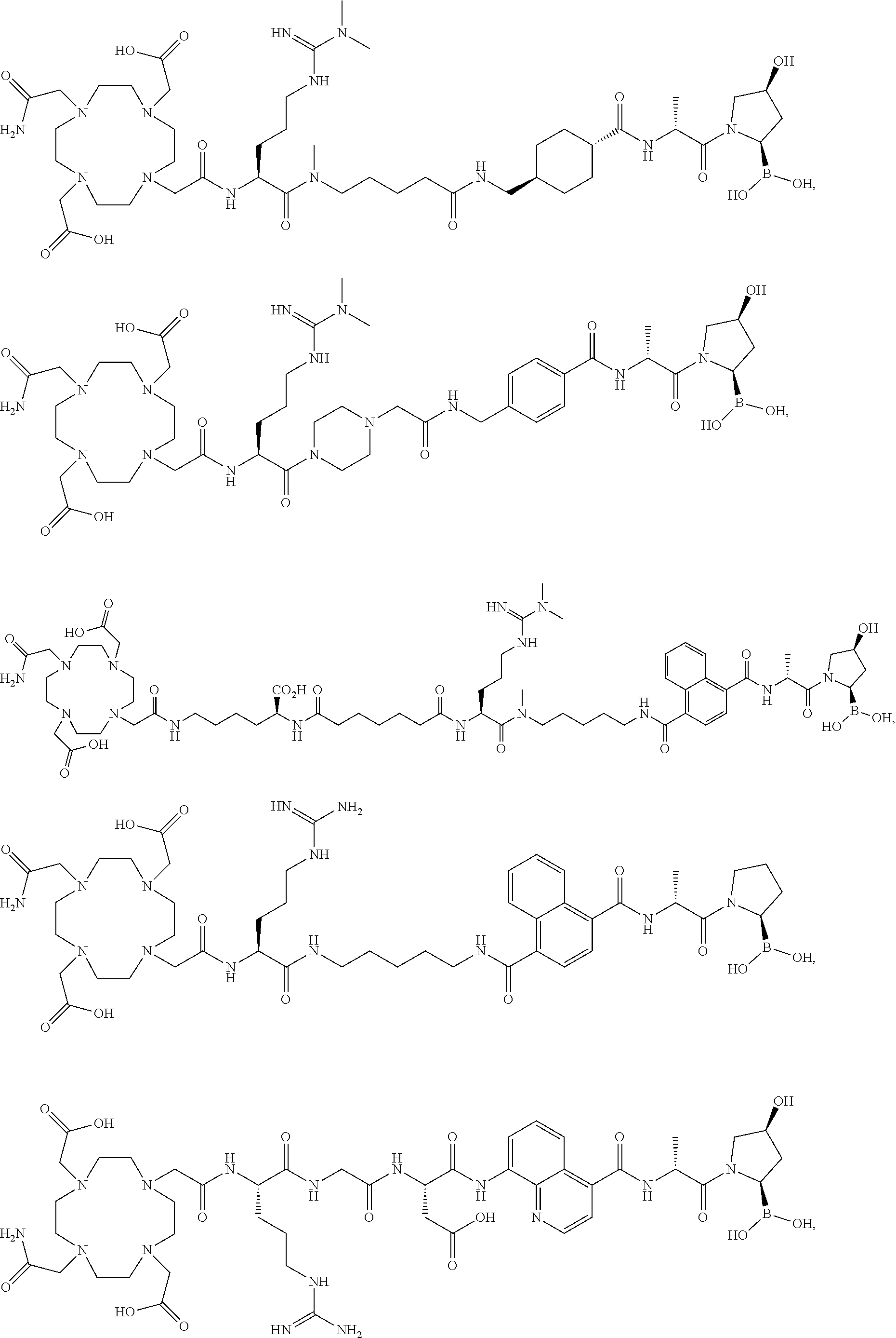

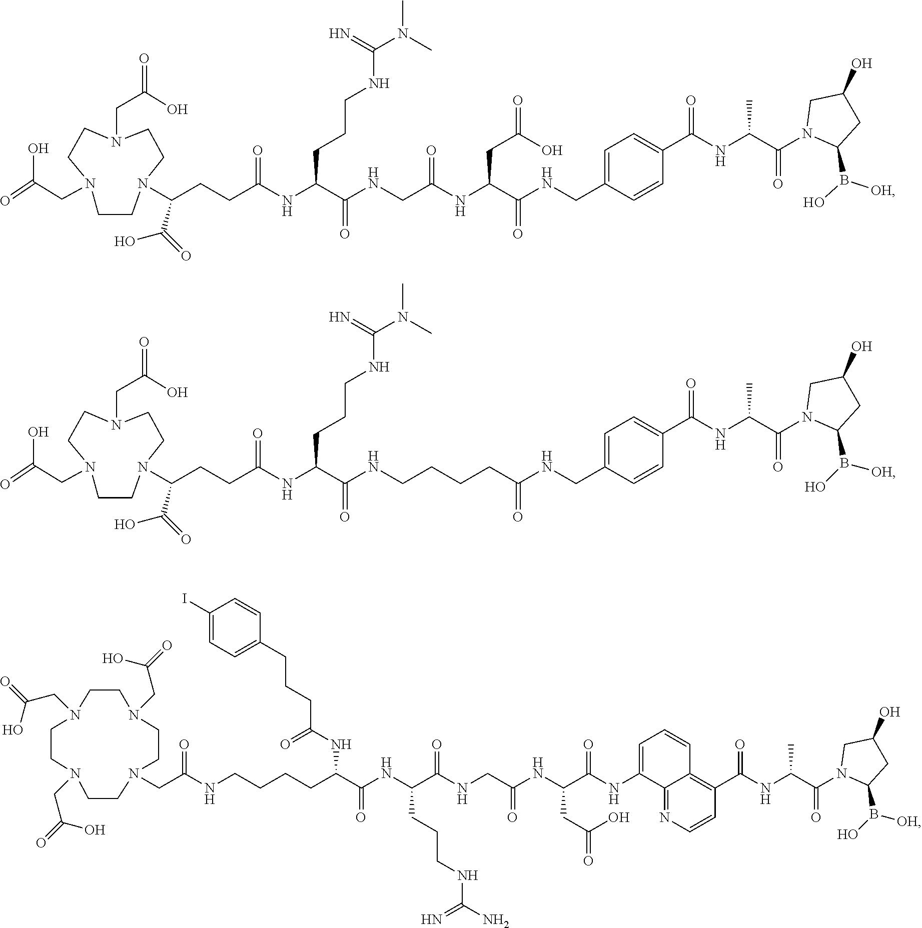

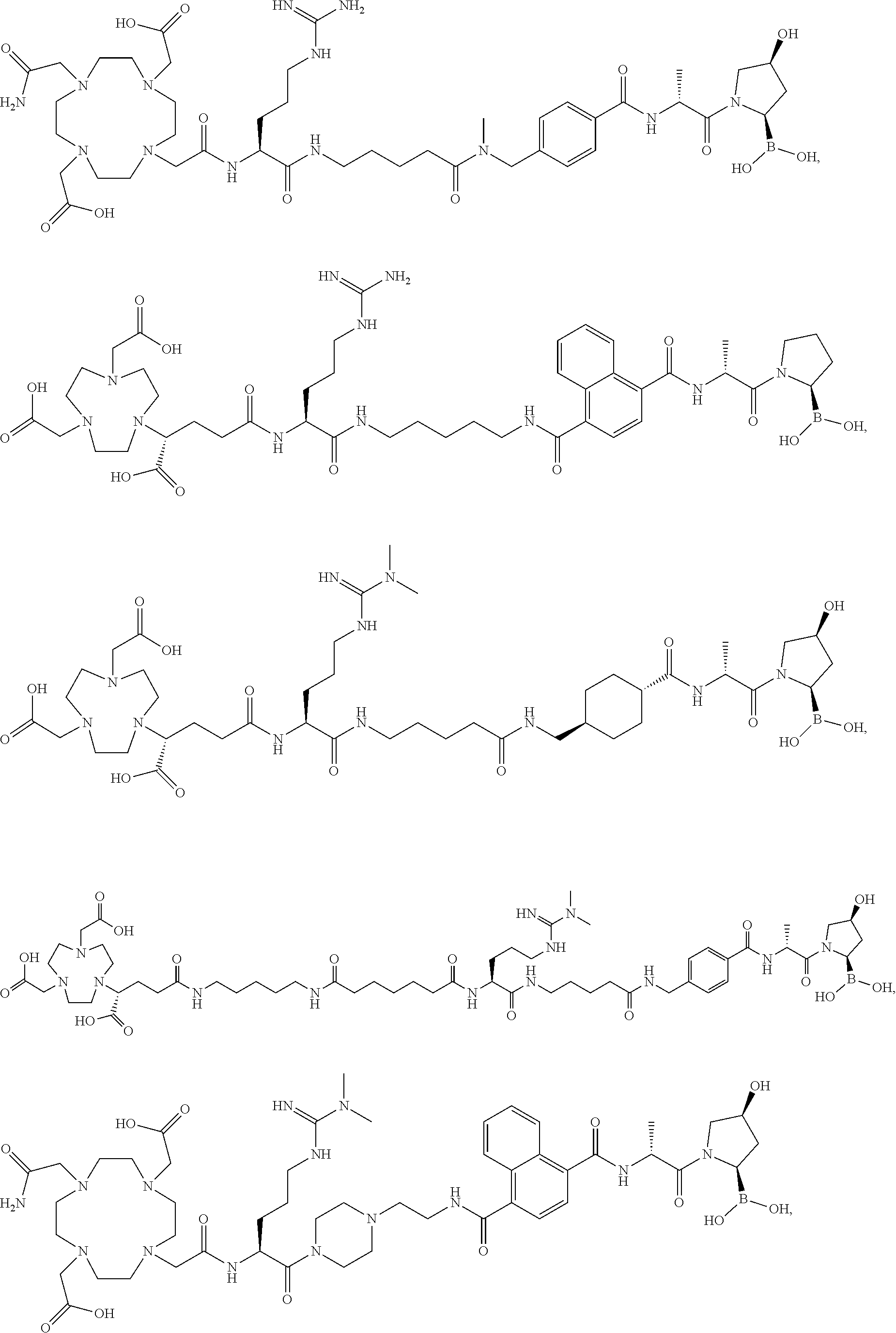

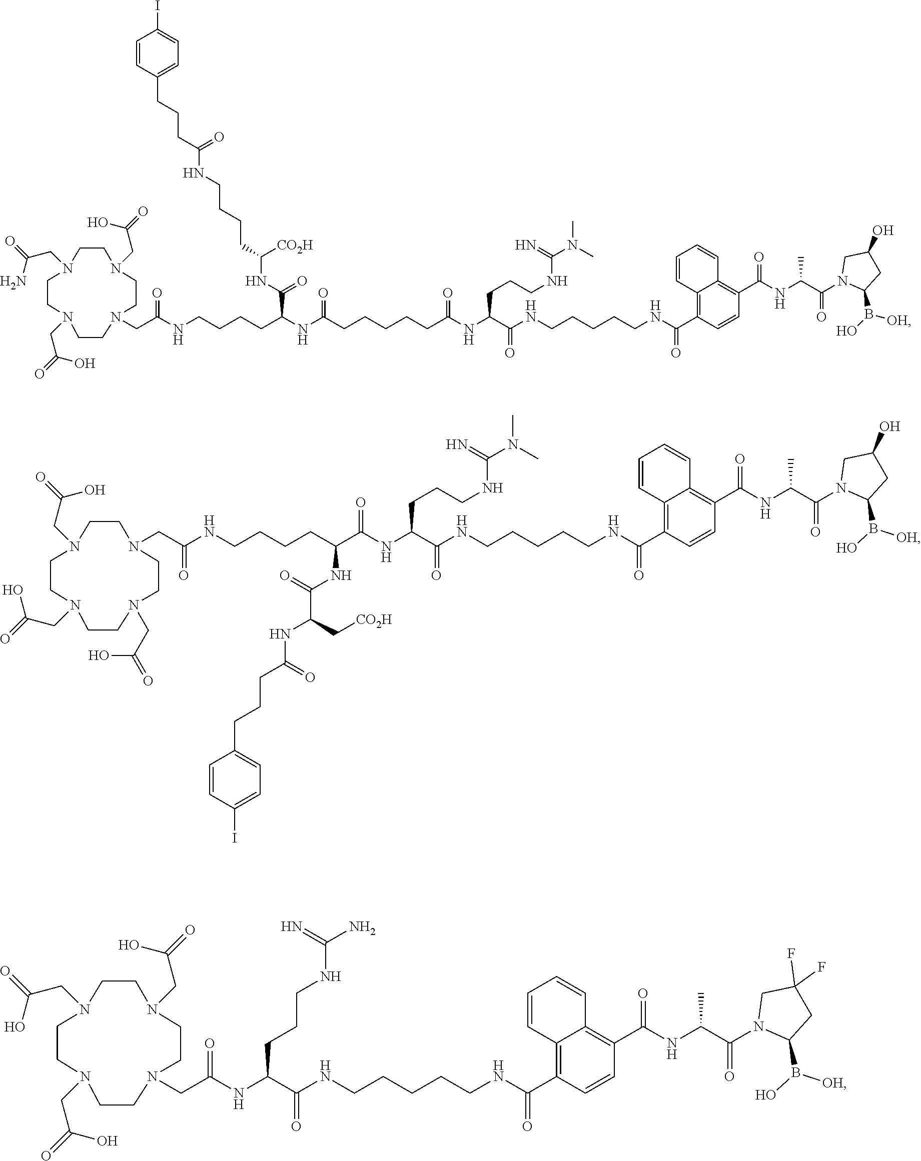

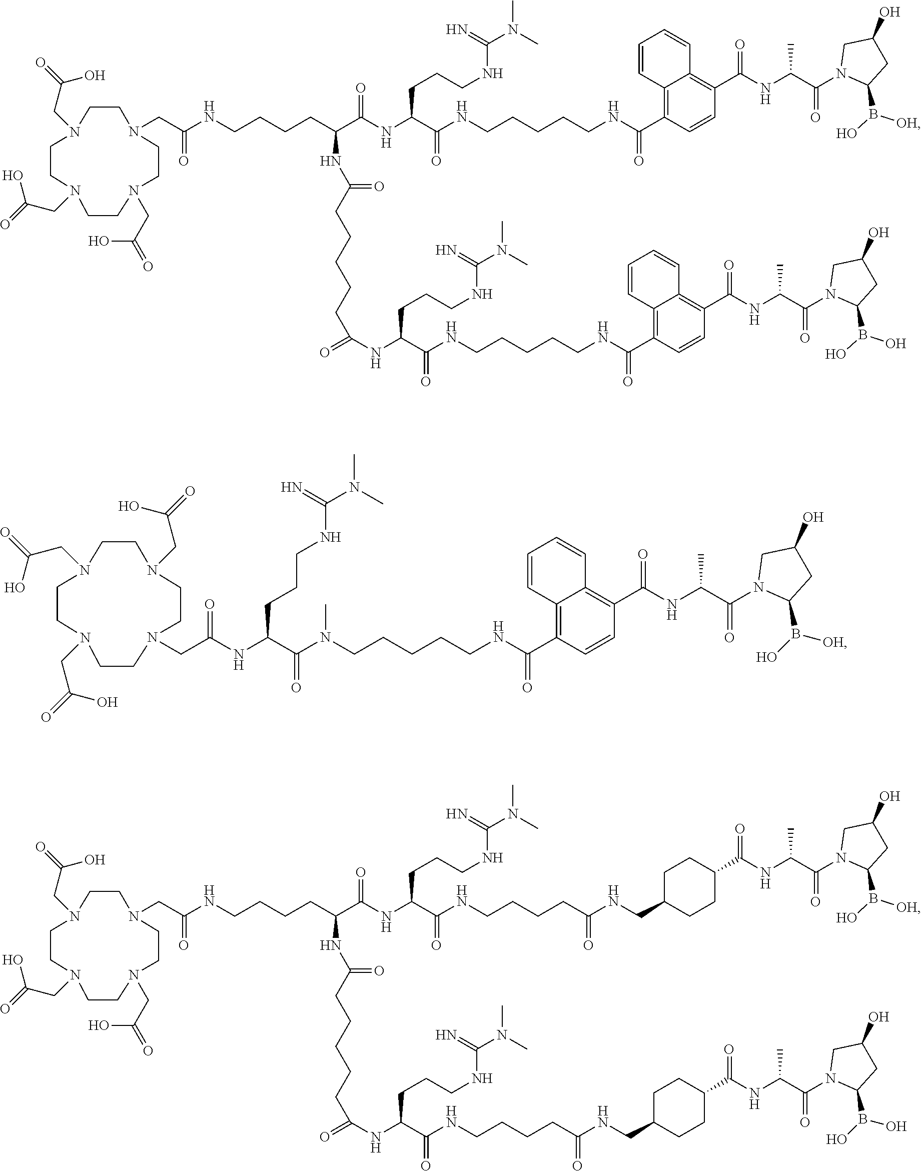

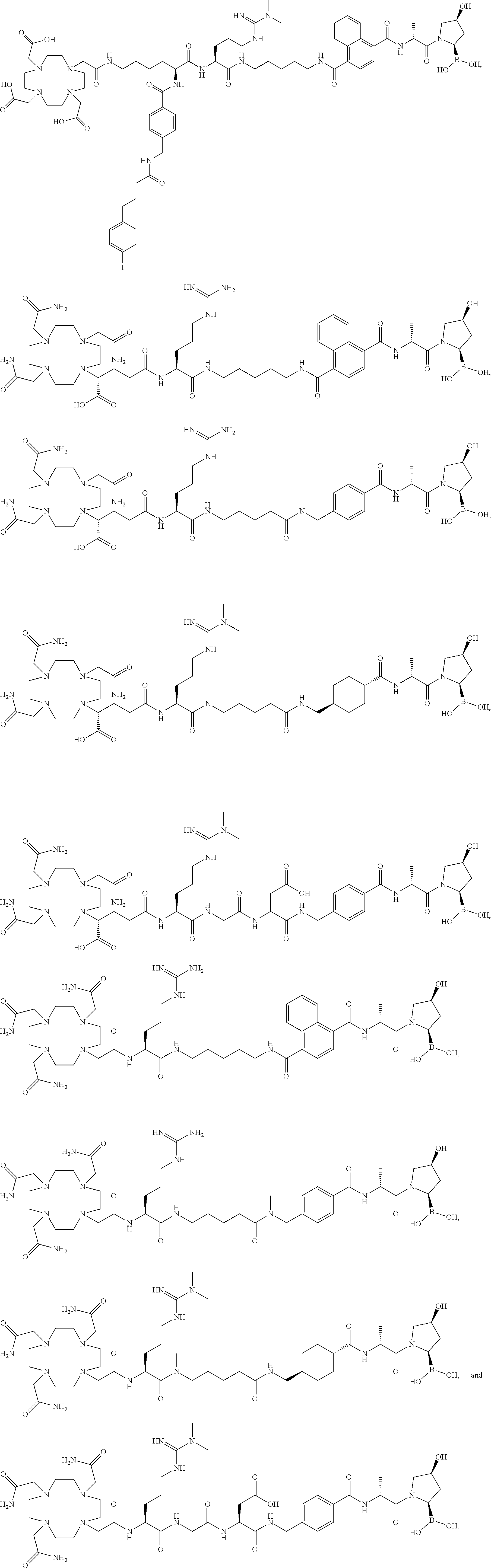

33-37. (canceled)

38. A pharmaceutical composition comprising a compound according to claim 1, or a pharmaceutically acceptable salt thereof; and a pharmaceutically acceptable carrier, diluent or excipient.

39. (canceled)

40. (canceled)

41. A method of treating cancer in a patient in need thereof, the method comprising administering to the patient an effective amount of a compound according to claim 1, or a pharmaceutically acceptable salt thereof.

42. The method of claim 41, further comprising administering to the patient an effective amount of one or more additional therapeutic agents selected from the group consisting of: a PD-1 inhibitor, a PD-L1 inhibitor, a CDK4/6 inhibitor, a PARP inhibitor, an EGFR inhibitor, a chemotherapeutic agent, and a platinum-based drug.

43-45. (canceled)

46. The method of claim 41, wherein the cancer is pancreatic ductal adenocarcinoma (PDAC).

47. (canceled)

48. The method of claim 41, wherein the cancer is characterized by the expression of Fibroblast Activation Protein (FAP) on a tumor, fibroblast, or pericyte within the patient.

49. (canceled)

50. The method according to claim 41, wherein the cancer is selected from the group consisting of lung cancer, pancreatic cancer, cervical cancer, esophageal cancer, endometrial cancer, ovarian cancer, cholangiocarcinoma, and colorectal cancer.

51.-55. (canceled)

56. The method of claim 41, wherein the compound is administered as the pharmaceutical composition according to claim 38.

57.-63. (canceled)

64. The method of claim 41, wherein treatment comprises inhibiting FAP-expressing cells in the patient.

65.-87. (canceled)
